SEQUENCE LISTING (1 sequence)

SEQ ID NO: 1 - protein, 686 aa, Homo sapiens
MRLLTLLGLLCGSVATPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAEDIDECQVAPGEAPTCDHHCHNHLGGFYCSCRAGYVLHRNKRTCSALCSGQVFTQRSGELSSPEYPRPYPKLSSCTYSISLEEGFSVILDFVESFDVETHPETLCPYDFLKIQTDREEHGPFCGKTLPHRIETKSNTVTITFVTDESGDHTGWKIHYTSTAQPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

(19) United States
(12) Patent Application Publication
(10) Pub. No.: US 2025/0122225 A1
(43) Pub. Date: Apr. 17, 2025

(54) MASP-2 INHIBITORS AND METHODS OF USE

(71) Applicant: Omeros Corporation, Seattle, WA (US)

(72) Inventors: Neil S. Cutshall, Snohomish, WA (US); Melinda Renee Davis, Seattle, WA (US); Jennifer Lynn Gage, Kenmore, WA (US); Sara Rebecca Goldstein, Seattle, WA (US); Do Yeon Kwon, Seattle, WA (US); Robert Huerta Lemus, Seattle, WA (US); Thomas L. Little, Seattle, WA (US); Markus Metz, Bellevue, WA (US); Jeremiah H. Nguyen, Kent, WA (US); Peter Kurt Nollert von Specht, Bainbridge Island, WA (US); Jennifer Tsoung, Seattle, WA (US); Sudheer Babu Vaddela, Bellevue, WA (US); Connor Weidle, Seattle, WA (US)

(73) Assignee: Omeros Corporation, Seattle, WA (US)

(21) Appl. No.: 18/907,356

(22) Filed: Oct. 4, 2024

Related U.S. Application Data
(60) Provisional application No. 63/588,644, filed on Oct. 6, 2023.

(51) Int. Cl. C07D 519/00; A61K 31/4985; C07D 487/04

(52) U.S. Cl. CPC C07D 519/00; A61K 31/4985; C07D 487/04

(57) ABSTRACT

The disclosure provides synthetic compositions useful as inhibitors of mannan-binding lectin-associated serine protease-2 (MASP-2), including inhibitors that selectively inhibit MASP-2 over thrombin, as well as methods for the manufacture and use thereof.

### CROSS-REFERENCE TO RELATED APPLICATION

This application claims the benefit of U.S. Application No. 63/588,644, filed on Oct. 6, 2023, the disclosure of which is hereby incorporated by reference in its entirety.

### STATEMENT REGARDING SEQUENCE LISTING

The Sequence Listing associated with this application is provided in XML format and is hereby incorporated by reference into the specification. The name of the XML file containing the sequence listing is 4278-P7US_Seq_List_ST26.xml. The XML file is 2641 bytes, was created on May 17, 2023, contains no new matter; and is being submitted electronically via Patent Center with the filing of the specification.

### BACKGROUND

The complement system plays a role in the inflammatory response and becomes activated upon tissue damage or microbial infection. Complement activation must be tightly regulated to ensure selective targeting of invading microorganisms and to avoid self-inflicted damage (Ricklin et al., Nat. Immunol. 11:785-797, 2010).

Currently, it is widely accepted that the complement system is activated through three distinct pathways: the classical pathway, the lectin pathway, and the alternative pathway. The classical pathway is usually triggered by a complex composed of host antibodies bound to a foreign particle (i.e., an antigen), and generally requires prior exposure to an antigen for the generation of a specific antibody response. Since activation of the classical pathway depends on a prior adaptive immune response by the host, the classical pathway is part of the acquired immune system response. In contrast, both the lectin and alternative pathways are independent of adaptive immunity, and are instead part of the innate immune system response.

Mannan-binding lectin-associated serine protease-2 (MASP-2) has been shown to be required for the function of the lectin pathway, one of the principal complement activation pathways (Vorup-Jensen et al., J. Immunol 165:2093-2100, 2000; Ambrus et al., J Immunol. 170:1374-1382, 2003; Schwaeble et al., PNAS 108:7523-7528, 2011).

Inhibition of MASP-2 appears to not interfere with the antibody-dependent classical complement activation pathway. As described in U.S. Pat. No. 9,011,860 (assigned to Omeros Corporation), which is hereby incorporated by reference in its entirety, a fully human monoclonal antibody targeting human MASP-2 has been generated, which binds to human MASP-2 with high affinity and blocks the lectin pathway complement activity.

MASP-2-dependent complement activation has been implicated as contributing to the pathogenesis of numerous acute and chronic disease states, including certain conditions caused by severe acute respiratory syndrome coronavirus 2 (SARS-CoV-2) infection.

Therefore, a need exists for compounds suitable for administration/treatment of subjects suffering from MASP-2-associated diseases and disorders, including diseases that are not suitably or efficiently treated with large molecule biologic inhibitors.

### SUMMARY

In some aspects, provided herein is a compound having Formula (I):

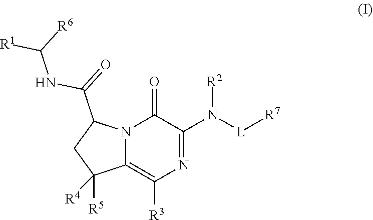

or a stereoisomer, tautomer, or pharmaceutically acceptable salt thereof, wherein:
R¹ is selected from a substituted or unsubstituted C₆-C₁₀ aryl and a substituted or unsubstituted 5-10-membered heteroaryl consisting of a 5-10-membered monocyclic or bicyclic ring system comprising at least one aromatic ring and one to six heteroatoms selected from the group consisting of nitrogen, oxygen, and sulfur, or
R¹ and R⁶, together with the carbon to which they are attached, form a C₃-C₆ cycloalkyl fused to a 5-10-membered heteroaryl or C₆-C₁₀ aryl, wherein the 5-10-membered heteroaryl or C₆-C₁₀ aryl, of the C₃-C₆ cycloalkyl fused to the 5-10-membered heteroaryl or C₆-C₁₀ aryl, is substituted or unsubstituted, and wherein the C₃-C₆ cycloalkyl, of the C₃-C₆ cycloalkyl fused to a 5-10-membered heteroaryl or C₆-C₁₀ aryl, is substituted or unsubstituted;
R² is hydrogen or C₁-C₃ alkyl, or R² and L, together with the nitrogen to which they are attached, form a substituted or unsubstituted 3-6-membered heterocyclyl;
L is —(CR^{8a}R^{8b})ₙ—, wherein each —(CR^{8a}R^{8b})— is the same or is different;
R³ is selected from the group consisting of hydrogen, substituted or unsubstituted C₁-C₃ alkyl, substituted or unsubstituted C₃-C₆ cycloalkyl, substituted or unsubstituted 5-6-membered saturated or unsaturated heterocyclyl, substituted or unsubstituted phenyl, substituted or unsubstituted 5-6-membered heteroaryl, halogen, and cyano;
R⁴ and R⁵ are each independently selected from the group consisting of hydrogen, substituted or unsubstituted C₁-C₆ alkyl, and substituted or unsubstituted benzyl, or R⁴ and R⁵, together with the carbon to which they are attached, form a substituted or unsubstituted C₃-C₆ cycloalkyl or a substituted or unsubstituted 5-6-membered cycloalkenyl;
R⁶ is selected from the group consisting of hydrogen, C₁-C₃ alkyl, C₃-cycloalkyl, and cyano;
R⁷ is selected from the group consisting of hydrogen, substituted or unsubstituted C₁-C₃ alkyl, substituted or unsubstituted 5-6-membered heteroalkyl, substituted or unsubstituted C₃-C₆ cycloalkyl, substituted or unsubstituted C₃-C₆ cycloalkyl fused with aryl, substituted or unsubstituted 3-6-membered heterocyclyl, substituted or unsubstituted C₆-C₁₀ aryl, substituted or unsubstituted C₆-C₁₀ aryl fused with C₅-C₆ cycloalkyl, substituted or unsubstituted 5-9-membered heteroaryl, substituted or unsubstituted 5-9-membered heteroaryl fused with C₅-C₆ cycloalkyl, alkoxy, and halogen, and wherein when R⁷ is hydrogen, n is 0;
R^{8a} and R^{8b} are each independently selected from the group consisting of hydrogen, C₁-C₄ linear alkyl, C₃-C₄ branched alkyl, substituted or unsubstituted C₃-C₆ cycloalkyl, hydroxyalkyl, and alkoxy; or R^{8a} and R^{8b} together, with the carbon to which they are attached, form a substituted or unsubstituted C₃-C₆ cycloalkyl or a substituted or unsubstituted 3-6-membered heterocyclyl; or n is 3, one of R^{8a} or R^{8b} from a first carbon is a bridging —CH₂— to a third carbon, and one of R^{8a} or R^{8b} from the third carbon is absent, to form a 1,3-cyclobutylene; and
n is 0, 1, 2, 3, or 4, and
wherein when one or more chiral center is present, each chiral center has (R) stereochemistry, (S) stereochemistry, or is a mixture thereof.

In some embodiments, provided herein is a pharmaceutical composition, comprising a compound of Formula (I), or a stereoisomer, tautomer, or pharmaceutically acceptable salt thereof, and a pharmaceutically acceptable carrier or excipient.

In some embodiments, provided herein is a method for inhibiting MASP-2 in a subject, comprising administering to the subject a compound of Formula (I), or a stereoisomer, tautomer, or pharmaceutically acceptable salt thereof, in an amount effective to inhibit MASP-2.

In some embodiments, provided herein is a method for treating a disease or disorder treatable by inhibiting MASP-2, comprising administering to a subject in need thereof a therapeutically effective amount of a compound of Formula (I), or a stereoisomer, tautomer, or pharmaceutically acceptable salt thereof.

In some embodiments, provided herein is a method of inhibiting MASP-2 dependent complement activation in a subject.

### DETAILED DESCRIPTION

Unless otherwise defined, all technical and scientific terms used herein have the same meaning as commonly understood by one of ordinary skill in the art to which this disclosure belongs. Although methods and materials similar or equivalent to those described herein can be used in the practice or testing of the subject matter of the present disclosure, suitable methods and materials are described below. In addition, the materials, methods, and examples are illustrative only and not intended to be limiting.

### I. Definitions

In certain embodiments herein, reference is made to features and aspects of the disclosure, including method steps. All possible combinations of such features and aspects within the embodiments of the disclosure are included, at least to the extent that such combinations are non-contradictory. For example, if an embodiment presents aspects A, B, and C, it is understood that this also discloses embodiments including both aspects A and B, both aspects B and C, and both aspects A and C, as well as an embodiment with aspects A, B, and C.

The terms “a,” “an,” or “the” not only include aspects with one member, but also include aspects with more than one member. For instance, the singular forms “a,” “an,” and “the” include plural referents unless the context clearly dictates otherwise.

The terms “about” and “approximately” refer to an acceptable degree of error for the quantity measured given the nature or precision of the measurements. Typical, exemplary degrees of error are within ±20 percent (%); preferably, within ±10%; and more preferably, within ±5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written support for a claim limitation of, e.g., “0.98X.” Alternatively, in biological systems, the terms “about” and “approximately” can mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated. When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11 mg/kg” is equivalent to “about 7, about 9, or about 11 mg/kg.”

The term “or” refers to an alternative and should in general be construed non-exclusively. For example, a claim to “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

The group “A or B” is equivalent to the group “selected from the group consisting of A and B.”

The linking term “comprising” or “comprise” is not closed. For example, “a composition comprising A” must include at least the component A, but it may also include one or more other components. For example, a claim to “a composition comprising A” would cover compositions that include A and B; A, B, and C; A, B, C, and D; A, B, C, D, and E; and the like. The term “comprising” therefore should in general be construed as not excluding additional ingredients.

The term “MASP-2” refers to mannan-binding lectin-associated serine protease-2. For example, human MASP-2 protein can have UniProt accession code O00187 (SEQ ID NO:1). The Serine Protease Domain (‘B-chain’=Mannan-binding lectin serine protease 2 B chain, based on UniProtKB—O00187 (MASP-2_HUMAN)) includes residues 445 to 686 (or consists of residues 445 to 686).

The term “MASP-2-dependent complement activation” refers to MASP-2-dependent activation of the lectin pathway, which occurs under physiological conditions (i.e., in the presence of Ca⁺⁺) leading to the formation of the lectin pathway C3 convertase C4b2a and upon accumulation of the C3 cleavage product C3b subsequently to the C5 convertase C4b2a(C3b)n.

The term “MASP-2-dependent complement-associated disease or disorder” refers to a disease or disorder that is associated with MASP-2-dependent complement activation.

The term “MASP-2-associated disease or disorder” refers to a disease or disorder that is associated with activation or activity of MASP-2, including MASP-2-dependent complement-associated disease or disorders, and wherein inhibition of MASP-2 is, or is expected to be, therapeutically beneficial.

Typically, the active site of serine proteases, such as MASP-2, is shaped as a cleft where the substrate or inhibitor binds.

The term “binding site” is an area on the protein wherein a compound can interact, such as a region in a cleft of MASP-2 or on the surface of MASP-2. The binding site or region may only partially overlap with the active site, or may not overlap with the active site at all, but may nevertheless render the MASP-2 molecule less active or inactive.

The term “lectin pathway” refers to complement activation that occurs via the specific binding of serum and non-serum carbohydrate-binding proteins including mannan-binding lectin (MBL), CL-11, and the ficolins (e.g., H-ficolin, M-ficolin, or L-ficolin).

The term “classical pathway” refers to complement activation that is triggered by an antibody bound to a foreign particle and requires binding of the recognition molecule C1q.

The term “a subject” includes all mammals, including without limitation, humans, non-human primates, dogs, cats, horses, sheep, goats, cows, rabbits, pigs, and rodents.

“Mammal” includes humans; domestic animals such as laboratory animals and household pets (e.g., cats, dogs, swine, cattle, sheep, goats, horses, rabbits); and non-domestic animals such as wildlife, and the like.

As used herein, the terms “disease,” “condition,” and “disorder” may be used interchangeably or may be different in that the particular malady, condition, disorder, or syndrome may not have a known causative agent (so that etiology has not yet been worked out) and it is therefore not yet recognized as a disease but only as an undesirable condition, disorder, or syndrome, or is a disruption of normal processes or functions, wherein a more or less specific set of symptoms has been identified by a clinician or researcher. In some embodiments, a disease is a pathological condition of an organ, a body part, or a system, resulting from various causes such as infection, genetic defect, or environmental stress that is characterized by an identifiable group of symptoms.

“Therapeutically effective amount,” “effective amount,” or “effective dose” refers to the amount of a compound of the disclosure that, when administered to a mammal (e.g., a human), is sufficient to effect treatment as defined herein, reduction in symptoms, or cure, of a disease or condition in the mammal, preferably a human. The amount of a compound of the disclosure which constitutes a “therapeutically effective amount” will vary depending on the compound; the condition and its severity; the manner of administration; and the age, weight, and genetics of the mammal to be treated, but can be determined routinely by one of ordinary skill in the art having regard to his own knowledge and to this disclosure.

The term “agent” refers to a compound or mixture of compounds that, when added to a composition, tends to produce an effect on the composition's properties. For example, a composition comprising a thickening agent is likely to be more viscous than an otherwise identical comparative composition that lacks the thickening agent.

A “synthetic” compound means a compound that is not naturally occurring or that has been synthesized by humans. Reference to a compound herein may be understood to include reference to synthetic compounds, unless the context indicates otherwise.

The expressions, “ambient temperature” and “room temperature,” as used herein, are understood in the art, and refer generally to a temperature, e.g., a reaction temperature, that is about the temperature of the room in which the reaction is carried out, e.g., a temperature from about 18° C. to about 30° C.

At various places in the present specification, certain features of the compounds are disclosed in groups or in ranges. It is specifically intended that such a disclosure include each and every individual sub-combination of the members of such groups and ranges. For example, the terms “C₁₋₆ alkyl” and “C₁-C₆ alkyl” are specifically intended to individually disclose (without limitation) methyl, ethyl, C₃ alkyl, C₄ alkyl, C₅ alkyl, and C₆ alkyl, including all linear and branched compositions (e.g., n-butyl, sec-butyl, isobutyl, and tert-butyl for C₄ alkyl).

The term “substituted” means that an atom or group of atoms formally replaces hydrogen as a “substituent” attached to another group. The term “substituted” means that at least one hydrogen atom is replaced with a non-hydrogen substituent. Additionally, a compound can be substituted with a hydrogen, and hydrogen can be a substituent. The term “substituted,” unless otherwise indicated, refers to any level of substitution, e.g., mono-, di-, tri-, tetra-, penta-, or higher substitution, where such substitution is permitted. The substituents are independently selected, and substitution may be at any chemically accessible position. It is to be understood that substitution at a given atom is limited by valency. A single divalent substituent, e.g., oxo, can replace two hydrogen atoms.

The phrase “optionally substituted” means substituted or unsubstituted.

The terms “Cₙ₋ₘ” and “Cₙ-Cₘ,” where n and m are integers, indicates a group that contains from n to m carbon atoms, includes both linear and branched configurations, and does not exclude substituents. Examples include C₁₋₄, C₁₋₆, and the like. The term is intended to expressly disclose every member in the range, i.e., Cₙ, Cₙ₊₁, Cₙ₊₂ . . . Cₘ₋₂, Cₘ₋₁, Cₘ. For example, C₁₋₆ is intended to disclose C₁, C₂, C₃, C₄, C₅, and C₆. As used herein, “Cₙ₋ₘ” means the same as “Cₙ-Cₘ.”

The term “n-membered,” where n is an integer (e.g., 6-membered), typically describes the number of ring-forming atoms in a moiety where the number of ring-forming atoms is n. The term “n-m membered” wherein n and m are integers (e.g., 6-10 membered) describes a range wherein the number of ring forming atoms is from n to m. For example, piperidinyl is an example of a 6-membered heterocyclyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

“Alkyl” refers to a straight or branched hydrocarbon group consisting solely of carbon and hydrogen atoms, containing no unsaturation, having from one to twelve carbon atoms, preferably one to eight carbon atoms, more preferably one to six carbon atoms, and which is attached to the molecule by a single bond. An alkyl group is an alkane with one C—H bond replaced by the point of attachment of the alkyl group to the remainder of the molecule. For example, representative alkyl groups can be methyl, ethyl, n-propyl, 1-methylethyl (iso-propyl), n-butyl, n-pentyl, 1,1-dimethylethyl (t-butyl), 3-methylhexyl, 2-methylhexyl, and the like. In some embodiments, alkyl groups can be unsubstituted or substituted with one or more substituents, which can be the same or can be different.

“Alkenyl” refers to a straight or branched hydrocarbon group consisting solely of carbon and hydrogen atoms, comprising one or more carbon-carbon double bonds, having from two to twelve carbon atoms, preferably two to eight carbon atoms, and which is attached to the molecule by a single bond. An alkenyl group is an alkene with one C—H bond replaced by the point of attachment of the alkenyl group to the remainder of the molecule. For example, representative alkenyl groups can be ethenyl, prop-1-enyl, but-1-enyl, pent-1-enyl, penta-1,4-dienyl, and the like. In some embodiments, alkenyl groups can be unsubstituted or substituted with one or more substituents, which can be the same or can be different.

“Alkynyl” refers to a straight-chain or branched hydrocarbon group consisting solely of carbon and hydrogen atoms, comprising one or more carbon-carbon triple bonds, having from two to twelve carbon atoms, preferably two to eight carbon atoms, and which is attached to the molecule by a single bond. An alkynyl group is an alkyne with one C—H bond replaced by the point of attachment of the alkynyl group to the remainder of the molecule. The term “Cₙ₋ₘ alkynyl” and “Cₙ-Cₘ alkynyl” refer to an alkynyl group having n to m carbons. Example alkynyl groups include, but are not limited to, ethynyl, propyn-1-yl, propyn-2-yl and the like. In some embodiments, the alkynyl moiety contains 2 to 6, 2 to 4, or 2 to 3 carbon atoms. In some embodiments, alkynyl groups can be unsubstituted or substituted with one or more substituents, which can be the same or can be different.

“Alkylene” or “alkylene chain” refers to a straight or branched divalent hydrocarbon chain linking the molecule to another group, or linking two parts of the molecule, and consists solely of carbon and hydrogen, contains no unsaturation, and has from one to twelve carbon atoms, e.g., methylene, ethylene, propylene, n-butylene, and the like. The alkylene chain is attached to the molecule through a single bond and to the other group through a different single bond, or is attached to two parts of the molecule through a single bond at each point of attachment. In some embodiments, alkylene groups can be unsubstituted or substituted with one or more substituents, which can be the same or can be different.

The term “heteroalkyl” refers to a 3- to 18-membered non-aromatic and non-cyclic alkyl group which comprises two to twelve carbon atoms and one to six heteroatoms selected from the group consisting of nitrogen, oxygen, and sulfur. The nitrogen, carbon, or sulfur atoms in the heteroalkyl group may be optionally oxidized; the nitrogen atom may be optionally quaternized; and the heteroalkyl group may be saturated or unsaturated (e.g., the heteroalkyl comprises one or more double bond, and which can alternatively be termed a “heteroalkenyl”). In some embodiments, heteroalkyl groups can be unsubstituted or substituted with one or more substituents, which can be the same or can be different.

The term “hydroxyalkyl” refers to an alkyl group as defined herein, and in which one or more of the hydrogen atoms has been replaced by a hydroxy group (i.e., —OH). The term “Cₙ₋ₘ hydroxyalkyl” refers to a Cₙ₋ₘ alkyl group having n to m carbon atoms and at least one hydroxy group. In some embodiments, the hydroxyalkyl group comprises one hydroxy group. In some embodiments, the hydroxyalkyl group comprises two or more hydroxy groups (e.g., a “dihydroxyalkyl”), wherein the hydroxy groups are bound to the same or different carbon atom(s). In certain aspects, the hydroxyalkyl group has 1, 2, 3, 4, 5, 6, or more hydroxy groups. Examples may include, but are not limited to, hydroxymethyl, 2-hydroxyethyl, and 1-hydroxyethyl. In some embodiments, hydroxyalkyl groups can be unsubstituted or substituted with one or more substituents, which can be the same or can be different.

“Alkoxy” refers to a group having the following formula “—O-alkyl,” wherein the alkyl group is as defined herein. Example alkoxy groups include methoxy, ethoxy, propoxy (e.g., n-propoxy and isopropoxy), t-butoxy, and the like. In some embodiments, the alkyl group has 1 to 6, 1 to 4, or 1 to 3 carbon atoms. In some embodiments, alkoxy groups can be unsubstituted or substituted with one or more substituents, which can be the same or can be different.

“Aminylalkyl” or “aminoalkyl” refers to an alkyl group as defined above, and in which one or more hydrogen atoms have been replaced by an aminyl group, or amino group (i.e., —NRR′ wherein R and R′ are each independently hydrogen, alkyl, alkenyl, or alkynyl, as defined herein). In some embodiments, the aminylalkyl comprises one aminyl group. In some embodiments, the aminyl group is —NH₂.

“Aryl” refers to a hydrocarbon ring system comprising hydrogen, 6 to 18 carbon atoms, and at least one aromatic ring. For purposes of this disclosure, the aryl group can be a monocyclic, bicyclic, tricyclic or tetracyclic ring system, which can include fused or bridged ring systems. Aryl groups include, but are not limited to, aryl groups derived from phenyl, benzene, naphthalene, anthracene, aceanthrylene, acenaphthylene, acephenanthrylene, azulene, chrysene, fluoranthene, fluorene, as-indacene, s-indacene, indane, indene, phenalene, phenanthrene, pleiadene, pyrene, and triphenylene. In some embodiments, aryl groups can be unsubstituted or substituted with one or more substituents, which can be the same or can be different. In embodiments wherein the aryl can be substituted, the aryl is substituted with one or more substituents, one substituent, two substituents, three substituents, four substituents, or five substituents, each substituent of which can be the same or can be different. In some embodiments, the term “aryl,” when substituted can include a fused ring substituent, e.g., a structure such as:

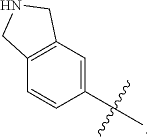 [SMILES: *c1ccc2c(c1)CNC2]

“Arylalkyl” or “aralkyl” refers to a group of -alkylene-aryl or -cycloalkylene-aryl wherein the alkylene or cycloalkylene group and aryl groups are as defined herein, respectively. In some embodiments, arylalkyl is —C₁₋₃ alkyl-C₆₋₁₀ aryl. In some embodiments, arylalkyl is —C₁₋₄ alkyl-C₆₋₁₀ aryl. In some embodiments, arylalkyl is —C₁₋₄ alkyl-phenyl. Examples include, but are not limited to, benzyl, 1-phenylethyl, 4-methylbenzyl, and 1,1,-dimethyl-1-phenylmethyl. In some embodiments, arylalkyl is unsubstituted or substituted on either the alkyl group or the aryl group, with one or more substituents which are the same or are different. In some embodiments, an arylalkyl is an optionally substituted benzyl. In some embodiments the arylalkyl group has the structure:

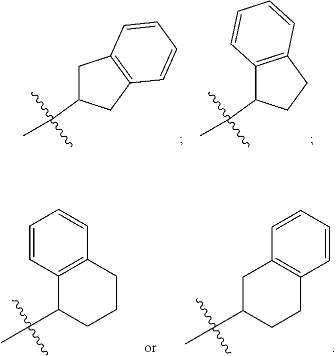 [SMILES: *C1Cc2ccccc2C1 ; *C1CCc2ccccc21 ; *C1CCCc2ccccc21 ; *C1CCc2ccccc2C1]

“Aryloxy” refers to a group with the formula —O-aryl, wherein the aryl is a group as herein defined. In some embodiments, the aryloxy group is —O—C₆₋₁₀ aryl. In some embodiments, the aryloxy is a substituted or unsubstituted phenyloxy (i.e., —O—C₆ aryl).

“Arylalkoxy” refers to a group with the formula -alkoxy-aryl, or —O-alkylene-aryl, wherein alkoxy and aryl are groups as defined herein. In some embodiments, arylalkoxy is —C₁₋₃ alkoxy-C₆₋₁₀ aryl. In some embodiments, arylalkoxy is —C₁₋₄ alkoxy-C₆₋₁₀ aryl. In some embodiments, arylalkoxy is —C₁₋₃ alkoxy-phenyl (e.g., —O-benzyl).

“Cycloalkyl” refers to a non-aromatic monocyclic or polycyclic hydrocarbon consisting solely of carbon and hydrogen atoms, which can include fused or bridged ring systems, having from three to fifteen carbon atoms, preferably having from three to ten carbon atoms, which is saturated, and which is attached to the rest of the molecule by a single bond. Monocyclic groups include, for example, cyclopropyl, cyclobutyl, cyclopentyl, cyclohexyl, cycloheptyl, and cyclooctyl. Polycyclic groups include, for example, adamantyl, norbornyl, decalinyl, and the like. In some embodiments, a cycloalkyl group is unsubstituted or substituted with one or more substituents, wherein the one or more substituents are independently the same or different.

“Cycloalkenyl” refers to a non-aromatic monocyclic or polycyclic hydrocarbon consisting solely of carbon and hydrogen atoms, which can include fused or bridged ring systems, having from three to fifteen carbon atoms, preferably having from three to ten carbon atoms, which is unsaturated, and which is attached to the rest of the molecule by a single bond. Monocyclic groups include, for example, cyclopentenyl and cyclohexenyl. In some embodiments, a cycloalkenyl group is unsubstituted or substituted with one or more substituents, wherein the one or more substituents are independently the same or different.

“Oxo” refers to a ═O group. For example, an oxo connected to a carbon atom forms a carbonyl group (i.e., C═O). Alternatively, when an oxo group is attached to a heteroatom, for example, a sulfoxide, sulfone group, or N-oxide group is formed.

“Amino” refers to a —NH₂ group.

“Acyl” refers to a —C(O)R group, wherein the R group can be an alkyl or aryl, as defined herein.

“Carboxy” refers to a —C(O)OH group.

“Carbonyl” refers to a C(═O) group, which also may be written as C(O).

“Cyano” or “nitrile” refers to a —C≡N group, which also may be written as —CN.

“Nitro” refers to a —NO₂ group.

“Hydroxy” or “hydroxyl” refers to an —OH group.

“Halo” or “halogen” refers to all of fluoro, chloro, bromo, and iodo, or to a subset of the halogen atoms (e.g., chloro, bromo, and iodo; chloro and bromo; or only chloro; etc.).

“Haloalkyl” refers to an alkyl group, as defined herein, wherein a hydrogen is substituted by one or more halo groups, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like. The alkyl part of the haloalkyl group may be unsubstituted or substituted with one or more substituents, wherein the one or more substituents are independently the same or different.

The term “haloalkoxy,” refers to a group of formula —O-haloalkyl, wherein the haloalkyl group is as defined herein. Example haloalkoxy groups include trifluoromethoxy, difluoromethoxy, pentafluoroethoxy, and the like.

“Heterocyclyl” refers to a 3- to 18-membered non-aromatic ring which comprises two to twelve carbon atoms and one to six heteroatoms selected from the group consisting of nitrogen, oxygen, and sulfur. Unless stated otherwise, the heterocyclyl may be a monocyclic, bicyclic, tricyclic, or tetracyclic ring system, which may include fused, bridged, and spiro ring systems; the nitrogen, carbon, or sulfur atoms in the heterocyclyl group may be optionally oxidized; the nitrogen atom may be optionally quaternized; and the heterocyclyl group may be saturated or unsaturated (e.g., the heterocyclyl comprises one or more double bond, and which can alternatively be termed a “heterocycloalkenyl”). Examples of heterocyclyl groups include, but are not limited to, azetidinyl, 3-azabicyclo[3.1.0]hexan-3-yl, 1-azaspiro[3.3]heptan-1-yl, 5-azaspiro[2.3]hexan-5-yl, 2-oxa-6-azaspiro[3.3]heptan-6-yl, 1-oxa-6-azaspiro[3.4]octan-6-yl, 1-oxa-6-azaspiro[3.3]heptan-6-yl, 6-oxa-1-azaspiro-[3.3]heptan-1-yl, 6-azaspiro[3.4]octan-6-yl, 7-oxa-2-azaspiro[3.5]nonan-2-yl, 2,6-diazaspiro[3.3]heptan-2-yl, dioxolanyl, dioxinyl, thienyl[1,3]dithianyl, decahydro-isoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, 1,2,4-thiadiazol-5(4H)-ylidene, tetrahydrofuryl, trioxanyl, trithianyl, triazinanyl, tetrahydropyranyl, thiomorpholinyl, thiamorpholinyl, 1-oxo-thiomorpholinyl, and 1,1-dioxo-thiomorpholinyl. In certain embodiments, a heterocyclyl group may be unsubstituted or substituted with one or more substituents, wherein the one or more substituents can independently be the same or different.

“Heteroaryl” refers to a 5-14-membered ring system comprising hydrogen atoms, one to thirteen carbon atoms, one to six heteroatoms selected from the group consisting of nitrogen, oxygen, and sulfur, and at least one aromatic ring. For purposes of this disclosure, the heteroaryl may be a monocyclic, bicyclic, tricyclic, or tetracyclic ring system, which may include fused or bridged ring systems; the nitrogen, carbon, or sulfur atoms in the heteroaryl may be optionally oxidized; and the nitrogen atom may be optionally quaternized. Examples include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, benzoxazolinonyl, benzimidazolthionyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, pteridinonyl, purinyl, pyrrolyl, pyrazolyl, pyridinyl, pyridinonyl, pyrazinyl, pyrimidinyl, pryrimidinonyl, pyridazinyl, pyrrolyl, pyrido[2,3-d]pyrimidinonyl, quinazolinyl, quinazolinonyl, quinoxalinyl, quinoxalinonyl, quinolinyl, isoquinolinyl, tetrahydroquinolinyl, thiazolyl, thiadiazolyl, thieno[3,2-d]pyrimidin-4-onyl, thieno[2,3-d]pyrimidin-4-onyl, triazolyl, tetrazolyl, triazinyl, and thiophenyl (i.e. thienyl). In some embodiments, the heteroaryl group can be unsubstituted or substituted. In embodiments wherein the heteroaryl is substituted, the heteroaryl is substituted with one or more substituents, each of which can be the same or can be different. In some embodiments, the term “heteroaryl” can include, e.g., structures such as:

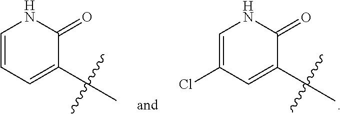 [SMILES: *c1ccc[nH]c1=O ; *c1cc(Cl)c[nH]c1=O]

“Heteroarylalkyl” refers to a group of the formula R¹⁰⁰R¹⁰¹ where R¹⁰⁰ is an alkylene as defined herein, and R¹⁰¹ is a heteroaryl as defined herein. When specifically stated in the specification, the heteroaryl moiety of the heteroarylalkyl may be unsubstituted or substituted as defined herein with one or more substituents, wherein the one or more substituents can independently be the same or different. In some embodiments, the alkylene moiety of the heteroarylalkyl may be unsubstituted or substituted as defined herein with one or more substituents, wherein the one or more substituents can independently be the same or different.

The compounds and methods of the present disclosure are intended to encompass all pharmaceutically acceptable isotopically labelled compounds of Formula (I), having one or more atoms replaced by an atom having a different atomic mass or mass number.

Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radio-labelled compounds can be useful to help determine or measure the effectiveness of the compounds, by characterizing, for example, the site or mode of action or binding affinity. Certain isotopically labelled compounds of Formula (I), for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with isotopes such as deuterium, i.e., ²H, may afford certain therapeutic advantages resulting from greater metabolic stability, for example, increased in vivo half-life or reduced dosage requirements, and hence may be preferred in some circumstances. In some embodiments, the compounds of the disclosure are enriched with deuterium. Such deuterated compounds can be achieved by methods known to one skilled in the art, such as exchanging protons with deuterium, or by synthesizing the molecule with deuterium-enriched starting materials.

Substitution with positron emitting isotopes, such as ¹¹C, ¹⁸F, ¹⁵O, and ¹³N, can be useful in Positron Emission Topography (PET) studies for examining substrate receptor occupancy. Isotopically labeled compounds of Formula (I) can generally be prepared by conventional techniques known to those skilled in the art, or by processes analogous to those described in the Examples as set out below using an appropriate isotopically labeled reagent in place of the non-labeled reagent.

“Substituted” refers to a group in which one or more hydrogens are optionally replaced by a non-hydrogen group to the extent that such substitution is chemically possible. Substituents can include, but are not limited to, halogens (F, Cl, Br, I), =O, ═N—CN, ═N—OR, =NR, OR, NR₂, SiR₃, SR, SO₂R, SO₂NR₂, NRSO₂R, NRCONR₂, NRC(O)OR, NRC(O)R, CN, C(O)OR, C(O)NR₂, OC(O)R, C(O)R, NO₂, C₁-C₆ alkyl, C₂-C₈ heteroalkyl, C₁-C₆ acyl, C₂-C₈ heteroacyl, C₂-C₈ alkenyl, C₂-C₈ heteroalkenyl, C₂-C₈ alkynyl, C₂-C₈ heteroalkynyl, C₆-C₁₀ aryl, or C₅-C₁₀ heteroaryl, wherein each R is independently H, C₁-C₆ alkyl, C₂-C₈ heteroalkyl, C₁-C₆ acyl, C₂-C₈ heteroacyl, C₂-C₈ alkenyl, C₂-C₈ heteroalkenyl, C₂-C₈ alkynyl, C₂-C₈ heteroalkynyl, C₆-C₁₀ aryl, or C₅-C₁₀ heteroaryl, and each R is optionally substituted with halogens (F, Cl, Br, I), =O, ═N—CN, =N—OR′, =NR′, OR′, NR′₂, SiR′₃, SR′, SO₂R′, SO₂NR′₂, NR′SO₂R′, NR′CONR′₂, NR′C(O)OR′, NR′C(O)R′, CN, C(O)OR′, C(O)NR′₂, OC(O)R′, C(O)R′, and NO₂, wherein each R′ is independently H, C₁-C₆ alkyl, C₂-C₈ heteroalkyl, C₁-C₈ acyl, C₂-C₈ heteroacyl, C₆-C₁₀ aryl, or C₅-C₁₀ heteroaryl. Alkyl, alkenyl, and alkynyl groups can also be substituted with C₁-C₆ acyl, C₂-C₈ heteroacyl, C₆-C₁₀ aryl, or C₅-C₁₀ heteroaryl, each of which can be substituted with substituents appropriate for the particular group. The substituents on an aryl or heteroaryl group can be further substituted with the suitable groups described herein. Thus, for example, an arylalkyl substituent may be substituted on the aryl moiety with substituents described herein for aryl groups. An arylalkyl substituent may alternatively be substituted on the alkyl moiety with substituents described herein for alkyl groups. An arylalkyl substituent may also be substituted on both the alkyl and aryl moieties.

“Optional” or “optionally” means that the subsequently described event of circumstances may or may not occur, and that the description includes instances where said event or circumstance occurs and instances in which said event or circumstance does not occur. For example, “optionally substituted aryl” or “substituted or unsubstituted aryl” means that the aryl may or may not be substituted, and that the description includes both substituted aryl groups and aryl groups lacking substitution (“unsubstituted”).

When a functional group is described as “optionally substituted,” and in turn, substituents on the functional group are also “optionally substituted” and so on, for the purposes of this disclosure, such iterations are limited to five. Such iterations are preferably limited to two.

“Optionally substituted,” as used herein, indicates that the particular group being described may have one, or more than one, hydrogen atoms replaced by a non-hydrogen group. In some optionally substituted groups, one hydrogen atom is replaced by a non-hydrogen group, e.g., C₁-C₆ alkyl, C₂-C₆ heteroalkyl, alkynyl, halogens (F, Cl, Br, I), N₃, OR, NR₂, SiR₃, SR, SO₂R, SO₂NR₂, NRSO₂R, NRCONR₂, NRC(O)OR, NRC(O)R, CN, C(O)OR, C(O)NR₂, OC(O)R, C(O)R, oxo, NO₂, C₁-C₆ acyl, C₂-C₈ heteroacyl, C₂-C₈ alkenyl, C₂-C₈ heteroalkenyl, C₂-C₈ alkynyl, C₂-C₈ heteroalkynyl, C₆-C₁₀ aryl, or C₅-C₁₀ heteroaryl, wherein each R is independently H, C₁-C₆ alkyl, 2-6-membered heteroalkyl, C₆-C₁₀ aryl, 5-10-membered heteroaryl, 5-9-membered heteroaryl, or as otherwise disclosed herein. In some optionally substituted groups, more than one hydrogen atom is replaced with one or more of the same or different non-hydrogen substituent, e.g., C₁-C₆ alkyl, C₂-C₆ heteroalkyl, alkynyl, halogens (F, Cl, Br, I), N₃, OR, NR₂, SiR₃, SR, SO₂R, SO₂NR₂, NRSO₂R, NRCONR₂, NRC(O)OR, NRC(O)R, CN, C(O)OR, C(O)NR₂, OC(O)R, C(O)R, oxo, NO₂, C₁-C₆ acyl, C₂-C₈ heteroacyl, C₂-C₈ alkenyl, C₂-C₈ heteroalkenyl, C₂-C₈ alkynyl, C₂-C₈ heteroalkynyl, C₆-C₁₀ aryl, or C₅-C₁₀ heteroaryl, wherein each R is independently H, C₁-C₆ alkyl, 2-6-membered heteroalkyl, C₆-C₁₀ aryl, 5-10-membered heteroaryl, 5-9-membered heteroaryl, or as otherwise disclosed herein. In some optionally substituted groups, all hydrogen atoms are replaced by the same or different non-hydrogen substituents, e.g., C₁-C₆ alkyl, C₂-C₆ heteroalkyl, alkynyl, halogens (F, Cl, Br, I), N₃, OR, NR₂, SiR₃, SR, SO₂R, SO₂NR₂, NRSO₂R, NRCONR₂, NRC(O)OR, NRC(O)R, CN, C(O)OR, C(O)NR₂, OC(O)R, C(O)R, oxo, NO₂, C₁-C₆ acyl, C₂-C₈ heteroacyl, C₂-C₈ alkenyl, C₂-C₈ heteroalkenyl, C₂-C₈ alkynyl, C₂-C₈ heteroalkynyl, C₆-C₁₀ aryl, or C₅-C₁₀ heteroaryl, wherein each R is independently H, C₁-C₆ alkyl, 2-6-membered heteroalkyl, C₆-C₁₀ aryl, 5-10-membered heteroaryl, 5-9-membered heteroaryl, or as otherwise disclosed herein. In some optionally substituted groups, one hydrogen atom, more than one hydrogen atom, or all hydrogen atoms, are replaced by a deuterium atom. Where an optional substituent is attached via a double bond, such as a carbonyl oxygen, or oxo (═O), the substituent group takes up two available valences, so the total number of substituents that may be included is reduced according to the number of available valences.

The compounds of the disclosure, or their pharmaceutically acceptable salts, may contain one or more stereocenter and may thus give rise to enantiomers, diastereomers, and other stereoisomeric forms that may be defined, in terms of absolute stereochemistry, as (R)- or (S)-, or as (D)- or (L)- for amino acids. The present disclosure is meant to include all such possible isomers, all combinations of such isomers, any ratio of stereoisomers, a racemic mixture, and optically pure forms. Optically active (+) and (−), (R)- and (S)-, or (D)- and (L)-isomers may be prepared using chiral synthons or chiral reagents, or resolved using conventional techniques, for example, chromatography or fractional crystallization. Conventional techniques for the preparation/isolation of individual enantiomers or diastereoisomers include chiral synthesis from a suitable optically pure precursor, or resolution of the mixture (e.g., racemate or the racemate of a salt or derivative) using, for example, chiral high-performance liquid chromatography (HPLC).

As an example, the following structure of Formula (I) can have chiral centers at each position indicated by “*,” wherein each position can have (R)-absolute stereochemistry, (S)-absolute stereochemistry, or can be a mixture of (R)- and (S)-stereoisomers at each position. When an absolute stereochemistry is not shown at a given chiral center, this disclosure is intended to include both (R)- and (S)-stereoisomers at the chiral carbon atom.

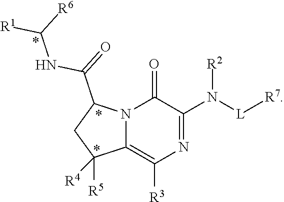

Additionally, stereoisomers can arise from the substituents defined by any one or more of R³, R⁴, R⁵, R⁶, R⁷, and/or L. For example, a stereoisomer can arise when a substituent (e.g., R³, R⁴, R⁵, and/or R⁷) is a “substituted C₃-C₆ cycloalkyl.”

This disclosure additionally includes stereoisomers which result from an optically pure form at a given chiral center, but in which a chemical reaction caused, or resulted in, epimerization such that after the chemical reaction, the molecule was rendered to any extent to no longer be optically pure at the chiral center. For example, in the following reaction:

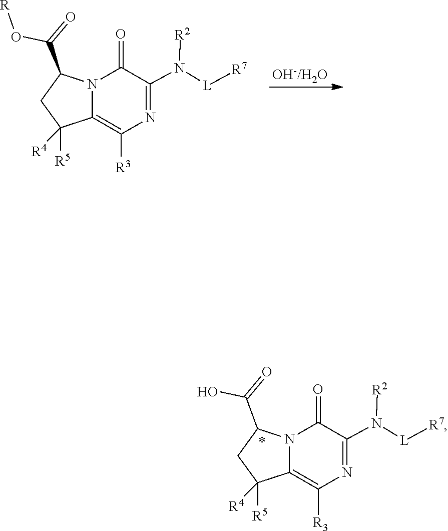

epimerization can occur to render the absolute stereochemistry shown in the starting molecule undefined in the reaction product. In the foregoing chemical reaction, the reaction product can retain the stereochemistry identified in the starting reagent, can be a mixture of (R)- or (S)-stereoisomers in any relative amount, or can be a racemic mixture having a 50/50 mixture of (R)- and (S)-stereoisomers. Products of such reaction may be disclosed herein without the stereochemistry shown, as above, or may be identified as a specific stereoisomer but may additionally comprise some amount of the other stereoisomer not shown.

A “stereoisomer” refers to a compound made up of the same atoms bonded by the same bonds, but having different three-dimensional structures, which are not interchangeable. A stereoisomer is present when a molecule comprises one or more chiral carbon atom. The present disclosure includes various stereoisomers and mixtures thereof. The present disclosure includes enantiomers, which refers to two stereoisomers whose molecules are non-superimposable mirror images of one another. See, e.g., Smith, M. B. and J. March, March's Advanced Organic Chemistry: Reactions, Mechanisms, and Structure, 6th edition (Wiley, 2007), for a detailed description of the structure and properties of enantiomers and stereoisomers. The present disclosure includes diastereomers, which refers to stereoisomers whose molecules are non-superimposable non-mirror images of each another.

When the compounds described herein contain olefinic double bonds or other centers giving rise to geometric asymmetry, and unless specified otherwise, it is intended that the compounds include both E and Z geometric isomers. Likewise, all tautomeric forms are also intended to be included.

A “tautomer” refers to a constitutional isomer wherein a proton can shift from one atom of a molecule to another atom of the same molecule (e.g., keto-enol). The present disclosure includes tautomers of any said compounds.

“Pharmaceutically acceptable carrier,” “pharmaceutically acceptable diluent,” “pharmaceutically acceptable excipient,” or “pharmaceutically acceptable carrier or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, pH adjusting agent, hydrogel, salt, inert solid, printed solid, or emulsifier, which has been approved by the United States Food and Drug Administration as being acceptable for use in humans or domestic animals.

“Pharmaceutically acceptable salt” includes both acid and base addition salts. Compounds, and salts thereof, can include more than one salt form. For example, the salt of a compound having two basic groups can include e.g., two trifluoroacetic acid salts, or one trifluoroacetic acid salt and one hydrochloride salt.

A pharmaceutically acceptable acid addition salt refers to salts which retain the biological effectiveness and properties of the free bases, which are not biologically, or otherwise, undesirable, and which are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

A pharmaceutically acceptable base addition salt refers to salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts, and the like. Preferred inorganic salts are ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins, and the like. Particularly preferred organic bases are isopropylamine, diethylamine, ethanolamine, trimethylamine, dicyclohexylamine, choline, and caffeine.

A “pharmaceutical composition” refers to a formulation of a compound of the disclosure and a medium generally accepted in the art for the delivery of the compound to mammals, e.g., humans. Such a medium includes all pharmaceutically acceptable carriers, diluents, or excipients therefor.

“Treating” or “treatment” as used herein covers the treatment of the disease or condition of interest in a mammal, preferably a human, having the disease or condition of interest or who may contract or develop the disease or condition of interest, and includes:
(a) preventing the disease or condition from occurring in a mammal, in particular, when such mammal is predisposed to the condition but has not yet been diagnosed as having it;
(b) inhibiting the disease or condition, i.e., arresting the disease or condition development;
(c) relieving (or ameliorating) the disease or condition, i.e., causing regression of the disease or condition; or
(d) relieving (or ameliorating) the symptoms resulting from the disease or condition, e.g., without addressing the underlying disease or condition.

The use of parentheses and brackets in substituent groups is used herein to conserve space. Accordingly, the use of parenthesis in a substituent group indicates that the group enclosed within the parentheses is attached directly to the atom preceding the parenthesis (e.g., —C(O)— represents a carbonyl). The use of brackets in a substituent group indicates that the group enclosed within the brackets is also attached directly to the atom preceding the brackets.

The chemical naming protocol and structure diagrams used herein are a modified form of the I.U.P.A.C. nomenclature system, using ChemDraw Professional Version 23.1.1.3 software program. For complex chemical names employed herein, a substituent group is named before the group to which it attaches. For example, cyclopropylethyl comprises an ethyl backbone with a cyclopropyl substituent. In chemical structure diagrams, all bonds are identified, except for some carbon atoms, which are assumed to be bonded to sufficient hydrogen atoms to complete the valency.

At certain places, the definitions or embodiments may refer to specific rings (e.g., an azetidine ring, a pyridine ring, etc.). Unless otherwise indicated, these rings can be attached to any ring member, provided that the valency of the atom is not exceeded.

When any two groups or two instances of the same substituent group are “independently” selected from a list of alternatives, the groups may be the same, or the groups may be different. For example, if R^{a} and R^{b} are independently selected from the group consisting of alkyl, fluoro, amino, and hydroxyalkyl, then a molecule with two R^{a} groups and two R^{b} groups could have all groups be an alkyl group (e.g., four different alkyl groups, four of the same alkyl groups, or four groups wherein some of the groups are the same and some are different). Alternatively, the first R^{a} could be alkyl, the second R^{a} could be fluoro, the first R^{b} could be hydroxyalkyl, and the second R^{b} could be amino (or any other substituents taken from the group). Alternatively, both R^{a} and the first R^{b} could be fluoro, while the second R^{b} could be alkyl (i.e., some pairs of substituent groups may be the same, while other pairs may be different). Unless otherwise indicated, if two or more groups having the same definition are present, but the definition provides for alternatives, it should be understood that each occurrence of the same group is independently selected from the possible alternatives. For example, if two or more R^{a} groups are present in a compound, and the definition of R^{a} provides that R^{a} can be A, B, or C, then it should be understood that each R^{a} group present in the compound is independently chosen from A, B, and C, so that the R^{a} groups present in the compound can be the same or different.

Compounds, and salts thereof, including pharmaceutically acceptable salts, can be found together with other substances such as water and solvents (e.g., hydrates and solvates), or can be isolated.

### II. Compounds

In some aspects, the present disclosure provides a compound having Formula (I):

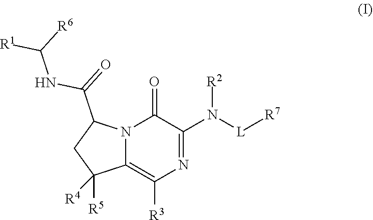

or a stereoisomer, tautomer, or pharmaceutically acceptable salt thereof, wherein:
R¹ is selected from a substituted or unsubstituted C₆-C₁₀ aryl and a substituted or unsubstituted 5-10-membered heteroaryl consisting of a 5-10-membered monocyclic or bicyclic ring system comprising at least one aromatic ring and one to six heteroatoms selected from the group consisting of nitrogen, oxygen, and sulfur, or
R¹ and R⁶, together with the carbon to which they are attached, form a C₃-C₆ cycloalkyl fused to a 5-10-membered heteroaryl or C₆-C₁₀ aryl, wherein the 5-10-membered heteroaryl or C₆-C₁₀ aryl, of the C₃-C₆ cycloalkyl fused to the 5-10-membered heteroaryl or C₆-C₁₀ aryl, is substituted or unsubstituted, and wherein the C₃-C₆ cycloalkyl, of the C₃-C₆ cycloalkyl fused to a 5-10-membered heteroaryl or C₆-C₁₀ aryl, is substituted or unsubstituted;
R² is hydrogen or C₁-C₃ alkyl, or R² and L, together with the nitrogen to which they are attached, form a substituted or unsubstituted 3-6-membered heterocyclyl;
L is —(CR^{8a}R^{8b})ₙ—, wherein each —(CR^{8a}R^{8b})— is the same or is different;
R³ is selected from the group consisting of hydrogen, substituted or unsubstituted C₁-C₃ alkyl, substituted or unsubstituted C₃-C₆ cycloalkyl, substituted or unsubstituted 5-6-membered saturated or unsaturated heterocyclyl, substituted or unsubstituted phenyl, substituted or unsubstituted 5-6-membered heteroaryl, halogen, and cyano;
R⁴ and R⁵ are each independently selected from the group consisting of hydrogen, substituted or unsubstituted C₁-C₆ alkyl, and substituted or unsubstituted benzyl, or R⁴ and R⁵, together with the carbon to which they are attached, form a substituted or unsubstituted C₃-C₆ cycloalkyl or a substituted or unsubstituted 5-6-membered cycloalkenyl;
R⁶ is selected from the group consisting of hydrogen, C₁-C₃ alkyl, C₃-cycloalkyl, and cyano;
R⁷ is selected from the group consisting of hydrogen, substituted or unsubstituted C₁-C₃ alkyl, substituted or unsubstituted 5-6-membered heteroalkyl, substituted or unsubstituted C₃-C₆ cycloalkyl, substituted or unsubstituted C₃-C₆ cycloalkyl fused with aryl, substituted or unsubstituted 3-6-membered heterocyclyl, substituted or unsubstituted C₆-C₁₀ aryl, substituted or unsubstituted C₆-C₁₀ aryl fused with C₅-C₆ cycloalkyl, substituted or unsubstituted 5-9-membered heteroaryl, substituted or unsubstituted 5-9-membered heteroaryl fused with C₅-C₆ cycloalkyl, alkoxy, and halogen, and when R⁷ is hydrogen, n is 0;
R^{8a} and R^{8b} are each independently selected from the group consisting of hydrogen, C₁-C₄ linear alkyl, C₃-C₄ branched alkyl, substituted or unsubstituted C₃-C₆ cycloalkyl, hydroxyalkyl, and alkoxy; or R^{8a} and R^{8b} together, with the carbon to which they are attached, form a substituted or unsubstituted C₃-C₆ cycloalkyl or a substituted or unsubstituted 3-6-membered heterocyclyl; or n is 3, one of R^{8a} or R^{8b} from a first carbon is a bridging —CH₂— to a third carbon, and one of R^{8a} or R^{8b} from the third carbon is absent, to form a 1,3-cyclobutylene; and
n is 0, 1, 2, 3, or 4, and
wherein when one or more chiral center is present, each chiral center has (R) stereochemistry, (S) stereochemistry, or is a mixture thereof.

In some embodiments, at least one of R³, R⁴, and R⁵ is not hydrogen, and R³ is not Cl when R¹ is

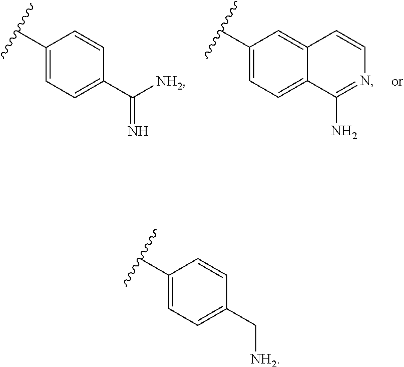 [SMILES: *c1ccc(C(=N)N)cc1 ; *c1ccc2c(N)nccc2c1 ; *c1ccc(CN)cc1]

In some embodiments, one or both of R³ and R⁶ is not hydrogen. In some embodiments, R⁶ is not hydrogen. In some embodiments, R³ is not hydrogen. In some embodiments, R⁶ is C₁-C₃ alkyl.

In some embodiments, one of R⁴ or R⁵ is substituted or unsubstituted benzyl. In some embodiments, R⁴ and R⁵, together with the carbon to which they are attached, form a substituted or unsubstituted C₃-C₆ cycloalkyl or a substituted or unsubstituted 5-6-membered cycloalkenyl.

In other aspects, the present disclosure provides a compound having Formula (I):

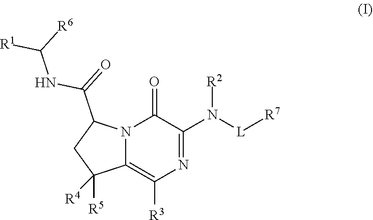

or a stereoisomer, tautomer, or pharmaceutically acceptable salt thereof, wherein:
R¹ is selected from a substituted or unsubstituted C₆-C₁₀ aryl and a substituted or unsubstituted 5-10-membered heteroaryl consisting of a 5-10-membered monocyclic or bicyclic ring system comprising at least one aromatic ring and one to six heteroatoms selected from the group consisting of nitrogen, oxygen, and sulfur, or
R¹ and R⁶, together with the carbon to which they are attached, form a C₃-C₆ cycloalkyl fused to a 5-10-membered heteroaryl or C₆-C₁₀ aryl, wherein the 5-10-membered heteroaryl or C₆-C₁₀ aryl, of the C₃-C₆ cycloalkyl fused to the 5-10-membered heteroaryl or C₆-C₁₀ aryl, is substituted or unsubstituted, and wherein the C₃-C₆ cycloalkyl, of the C₃-C₆ cycloalkyl fused to a 5-10-membered heteroaryl or C₆-C₁₀ aryl, is substituted or unsubstituted;
R² is hydrogen or C₁-C₃ alkyl, or R² and L, together with the nitrogen to which they are attached, form a substituted or unsubstituted 3-6-membered heterocyclyl;
L is —(CR^{8a}R^{8b})ₙ—, wherein each —(CR^{8a}R^{8b})— is the same or is different;
R³ is selected from the group consisting of hydrogen, substituted or unsubstituted C₁-C₃ alkyl, substituted or unsubstituted C₃-C₆ cycloalkyl, substituted or unsubstituted 5-6-membered saturated or unsaturated heterocyclyl, substituted or unsubstituted phenyl, substituted or unsubstituted 5-6-membered heteroaryl, halogen, and cyano;
R⁴ and R⁵ are each independently selected from the group consisting of hydrogen, substituted or unsubstituted C₁-C₆ alkyl, and substituted or unsubstituted benzyl, or R⁴ and R⁵, together with the carbon to which they are attached, form a substituted or unsubstituted C₃-C₆ cycloalkyl or a substituted or unsubstituted 5-6-membered cycloalkenyl;
R⁶ is selected from the group consisting of hydrogen, C₁-C₃ alkyl, C₃-cycloalkyl, and cyano;
R⁷ is selected from the group consisting of hydrogen, substituted or unsubstituted C₁-C₃ alkyl, substituted or unsubstituted 5-6-membered heteroalkyl, substituted or unsubstituted C₃-C₆ cycloalkyl, substituted or unsubstituted C₃-C₆ cycloalkyl fused with aryl, substituted or unsubstituted 3-6-membered heterocyclyl, substituted or unsubstituted C₆-C₁₀ aryl, substituted or unsubstituted C₆-C₁₀ aryl fused with C₅-C₆ cycloalkyl, substituted or unsubstituted 5-9-membered heteroaryl, substituted or unsubstituted 5-9-membered heteroaryl fused with C₅-C₆ cycloalkyl, alkoxy, and halogen, wherein when R⁷ is hydrogen, n is 0;
R^{8a} and R^{8b} are each independently selected from the group consisting of hydrogen, C₁-C₄ linear alkyl, C₃-C₄ branched alkyl, substituted or unsubstituted C₃-C₆ cycloalkyl, hydroxyalkyl, and alkoxy; or R^{8a} and R^{8b} together, with the carbon to which they are attached, form a substituted or unsubstituted C₃-C₆ cycloalkyl or a substituted or unsubstituted 3-6-membered heterocyclyl; or n is 3, one of R^{8a} or R^{8b} from a first carbon is a bridging —CH₂— to a third carbon, and one of R^{8a} or R^{8b} from the third carbon is absent, to form a 1,3-cyclobutylene; and
n is 0, 1, 2, 3, or 4,
wherein when one or more chiral center is present, each chiral center has (R) stereochemistry, (S) stereochemistry, or is a mixture thereof, and
provided that at least one of R³, R⁴, and R⁵ is not hydrogen, and
provided that R³ is not Cl when R¹ is

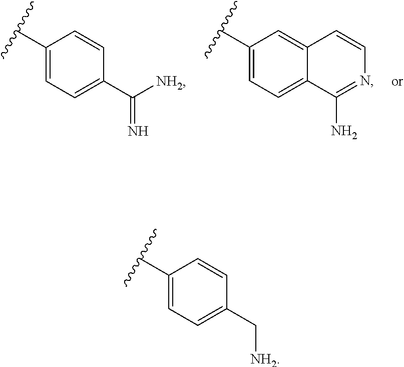 [SMILES: *c1ccc(C(=N)N)cc1 ; *c1ccc2c(N)nccc2c1 ; *c1ccc(CN)cc1]

In some embodiments, the present disclosure provides a compound of Formula (I):

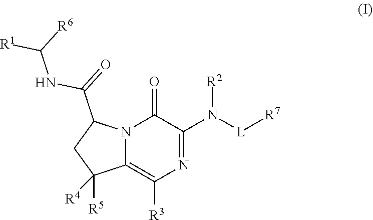

or a stereoisomer, tautomer, or pharmaceutically acceptable salt thereof, wherein:
R¹ is selected from a substituted or unsubstituted C₆-C₁₀ aryl and a substituted or unsubstituted 5-10-membered heteroaryl consisting of a 5-10-membered monocyclic or bicyclic ring system comprising at least one aromatic ring and one to six heteroatoms selected from the group consisting of nitrogen, oxygen, and sulfur, or
R¹ and R⁶, together with the carbon to which they are attached, form a C₃-C₆ cycloalkyl fused to a 5-10-membered heteroaryl or C₆-C₁₀ aryl, wherein the 5-10-membered heteroaryl or C₆-C₁₀ aryl, of the C₃-C₆ cycloalkyl fused to the 5-10-membered heteroaryl or C₆-C₁₀ aryl, is substituted or unsubstituted, and wherein the C₃-C₆ cycloalkyl, of the C₃-C₆ cycloalkyl fused to a 5-10-membered heteroaryl or C₆-C₁₀ aryl, is substituted or unsubstituted;
R² is hydrogen or C₁-C₃ alkyl, or R² and L, together with the nitrogen to which they are attached, form a substituted or unsubstituted 3-6-membered heterocyclyl;
L is —(CR^{8a}R^{8b})ₙ—, wherein each —(CR^{8a}R^{8b})— is the same or is different;
R³ is selected from the group consisting of hydrogen, substituted or unsubstituted C₁-C₃ alkyl, substituted or unsubstituted C₃-C₆ cycloalkyl, substituted or unsubstituted 5-6-membered saturated or unsaturated heterocyclyl, substituted or unsubstituted phenyl, substituted or unsubstituted 5-6-membered heteroaryl, halogen, and cyano;
R⁴ and R⁵ are each independently selected from the group consisting of hydrogen, substituted or unsubstituted C₁-C₆ alkyl, and substituted or unsubstituted benzyl, or R⁴ and R⁵, together with the carbon to which they are attached, form a substituted or unsubstituted C₃-C₆ cycloalkyl or a substituted or unsubstituted 5-6-membered cycloalkenyl;
R⁶ is selected from the group consisting of hydrogen, C₁-C₃ alkyl, C₃-cycloalkyl, and cyano;
R⁷ is selected from the group consisting of hydrogen, substituted or unsubstituted C₁-C₃ alkyl, substituted or unsubstituted 5-6-membered heteroalkyl, substituted or unsubstituted C₃-C₆ cycloalkyl, substituted or unsubstituted C₃-C₆ cycloalkyl fused with aryl, substituted or unsubstituted 3-6-membered heterocyclyl, substituted or unsubstituted C₆-C₁₀ aryl, substituted or unsubstituted C₆-C₁₀ aryl fused with C₅-C₆ cycloalkyl, substituted or unsubstituted 5-9-membered heteroaryl, substituted or unsubstituted 5-9-membered heteroaryl fused with C₅-C₆ cycloalkyl, alkoxy, and halogen, wherein when R⁷ is hydrogen, n is 0;
R^{8a} and R^{8b} are each independently selected from the group consisting of hydrogen, C₁-C₄ linear alkyl, C₃-C₄ branched alkyl, substituted or unsubstituted C₃-C₆ cycloalkyl, hydroxyalkyl, and alkoxy; or R^{8a} and R^{8b} together, with the carbon to which they are attached, form a substituted or unsubstituted C₃-C₆ cycloalkyl or a substituted or unsubstituted 3-6-membered heterocyclyl; or n is 3, one of R^{8a} or R^{8b} from a first carbon is a bridging —CH₂— to a third carbon, and one of R^{8a} or R^{8b} from the third carbon is absent, to form a 1,3-cyclobutylene; and
n is 0, 1, 2, 3, or 4,
wherein when one or more chiral center is present, each chiral center has (R) stereochemistry, (S) stereochemistry, or is a mixture thereof,
provided that at least one of R³, R⁴, and R⁵ is not hydrogen,
provided that R³ is not Cl when R¹ is

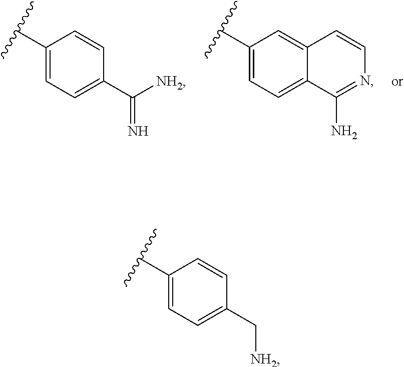 [SMILES: *c1ccc(C(=N)N)cc1 ; *c1ccc2c(N)nccc2c1 ; *c1ccc(CN)cc1]

and
provided that one or both of R³ and R⁶ is not H; one of R⁴ or R⁵ is substituted or unsubstituted benzyl; or R⁴ and R⁵, together with the carbon to which they are attached, form a substituted or unsubstituted 3-6-membered cycloalkyl or a substituted or unsubstituted 5-6-membered cycloalkenyl.

In yet other aspects, the present disclosure provides a compound having Formula (I):

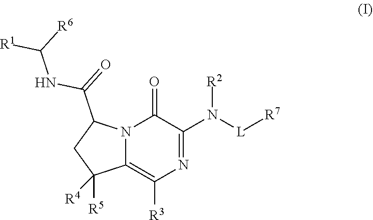

or a stereoisomer, tautomer, or pharmaceutically acceptable salt thereof, wherein:
R¹ is selected from a substituted or unsubstituted C₆-C₁₀ aryl and a substituted or unsubstituted 5-10-membered heteroaryl consisting of a 5-10-membered monocyclic or bicyclic ring system comprising at least one aromatic ring and one to six heteroatoms selected from the group consisting of nitrogen, oxygen, and sulfur, or
R¹ and R⁶, together with the carbon to which they are attached, form a C₃-C₆ cycloalkyl fused to a 5-10-membered heteroaryl or C₆-C₁₀ aryl, wherein the 5-10-membered heteroaryl or C₆-C₁₀ aryl, of the C₃-C₆ cycloalkyl fused to the 5-10-membered heteroaryl or C₆-C₁₀ aryl, is substituted or unsubstituted, and wherein the C₃-C₆ cycloalkyl, of the C₃-C₆ cycloalkyl fused to a 5-10-membered heteroaryl or C₆-C₁₀ aryl, is substituted or unsubstituted;
R² is hydrogen or C₁-C₃ alkyl, or R² and L, together with the nitrogen to which they are attached, form a substituted or unsubstituted 3-6-membered heterocyclyl;
L is —(CR^{8a}R^{8b})ₙ—, wherein each —(CR^{8a}R^{8b})— is the same or is different;
R³ is selected from the group consisting of hydrogen, substituted or unsubstituted C₁-C₃ alkyl, substituted or unsubstituted C₃-C₆ cycloalkyl, substituted or unsubstituted 5-6-membered saturated or unsaturated heterocyclyl, substituted or unsubstituted phenyl, substituted or unsubstituted 5-6-membered heteroaryl, halogen, and cyano;
R⁴ and R⁵ are each independently selected from the group consisting of hydrogen, substituted or unsubstituted C₁-C₆ alkyl, and substituted or unsubstituted benzyl, or R⁴ and R⁵, together with the carbon to which they are attached, form a substituted or unsubstituted C₃-C₆ cycloalkyl or a substituted or unsubstituted 5-6-membered cycloalkenyl;
R⁶ is selected from the group consisting of C₁-C₃ alkyl, C₃-cycloalkyl, and cyano;
R⁷ is selected from the group consisting of hydrogen, substituted or unsubstituted C₁-C₃ alkyl, substituted or unsubstituted 5-6-membered heteroalkyl, substituted or unsubstituted C₃-C₆ cycloalkyl, substituted or unsubstituted C₃-C₆ cycloalkyl fused with aryl, substituted or unsubstituted 3-6-membered heterocyclyl, substituted or unsubstituted C₆-C₁₀ aryl, substituted or unsubstituted C₆-C₁₀ aryl fused with C₅-C₆ cycloalkyl, substituted or unsubstituted 5-9-membered heteroaryl, substituted or unsubstituted 5-9-membered heteroaryl fused with C₅-C₆ cycloalkyl, alkoxy, and halogen, and when R⁷ is hydrogen, n is 0;
R^{8a} and R^{8b} are each independently selected from the group consisting of hydrogen, C₁-C₄ linear alkyl, C₃-C₄ branched alkyl, substituted or unsubstituted C₃-C₆ cycloalkyl, hydroxyalkyl, and alkoxy; or R^{8a} and R^{8b} together, with the carbon to which they are attached, form a substituted or unsubstituted C₃-C₆ cycloalkyl or a substituted or unsubstituted 3-6-membered heterocyclyl; or n is 3, one of R^{8a} or R^{8b} from a first carbon is a bridging —CH₂— to a third carbon, and one of R^{8a} or R^{8b} from the third carbon is absent, to form a 1,3-cyclobutylene; and
n is 0, 1, 2, 3, or 4, and
wherein when one or more chiral center is present, each chiral center has (R) stereochemistry, (S) stereochemistry, or is a mixture thereof.

In some embodiments, the compound of Formula (I) has Formula (I-1), (I-2), (I-3), (I-4), (I-5), (I-6), (I-7), (I-8), or a combination thereof:

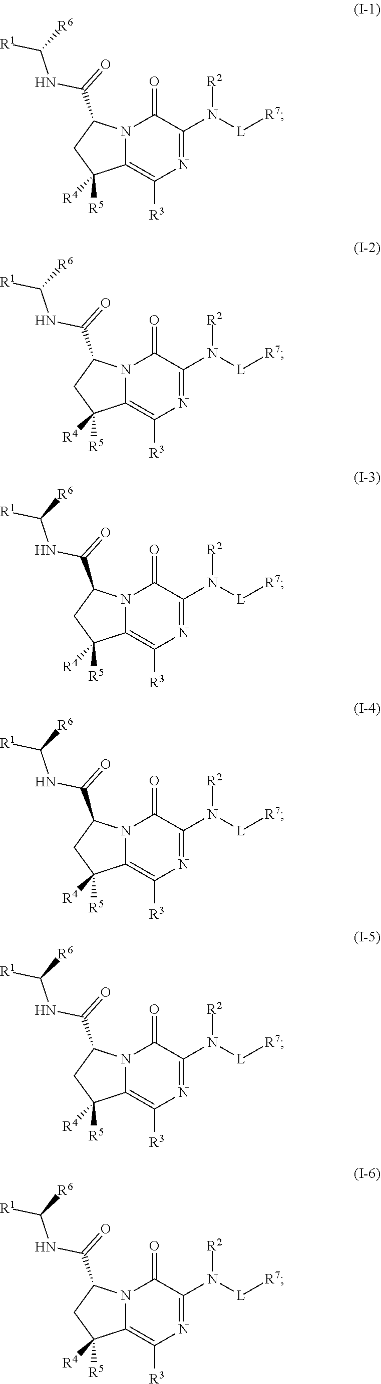

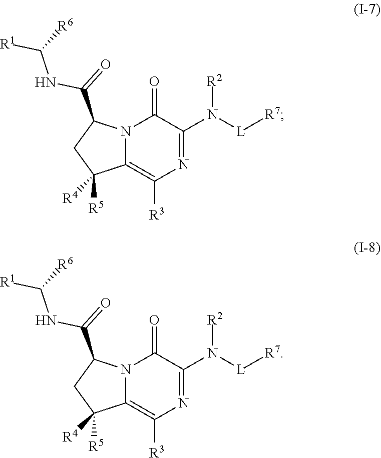

In some embodiments, Formula (I) is (I-3), (I-4), or a combination thereof.

In some embodiments of Formula (I), R⁶ is hydrogen. In some embodiments of Formula (I), R⁶ is C₁-C₃ alkyl. In some embodiments of Formula (I), R⁶ is methyl. In some embodiments of Formula (I), R⁶ is ethyl. In some embodiments of Formula (I), R⁶ is n-propyl or isopropyl. In some embodiments of Formula (I), R⁶ is C₃ cycloalkyl. In some embodiments of Formula (I), R⁶ is cyano.

In some embodiments, R⁶ is not hydrogen.

In some embodiments, the compound of Formula (I) has Formula (I-A):

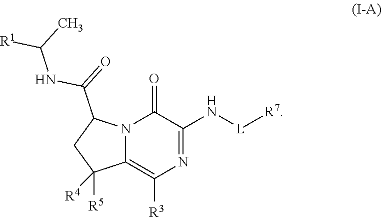

In some embodiments of Formula (I), R⁶ and R¹, together with the carbon to which they are attached, form a C₃-C₆ cycloalkyl fused to a substituted or unsubstituted 5-10-membered heteroaryl. In some embodiments of Formula (I), R⁶ and R¹, together with the carbon to which they are attached, form a C₃-C₆ cycloalkyl fused to a substituted or unsubstituted C₆-C₁₀ aryl. In some embodiments of Formula (I), R⁶ and R¹, together with the carbon to which they are attached, form a C₃-C₆ cycloalkyl fused to a substituted or unsubstituted phenyl. In some embodiments, the C₃-C₆ cycloalkyl of the C₃-C₆ cycloalkyl fused to a 5-10-membered heteroaryl is unsubstituted. In some embodiments, the C₃-C₆ cycloalkyl of the C₃-C₆ cycloalkyl fused to a C₆-C₁₀ aryl is unsubstituted. In some embodiments, the C₃-C₆ cycloalkyl of the C₃-C₆ cycloalkyl fused to a phenyl is unsubstituted. In some embodiments, the C₃-C₆ cycloalkyl of the C₃-C₆ cycloalkyl fused to a 5-10-membered heteroaryl is substituted with C₁-C₃ alkyl, alkoxy, or halogen. In some embodiments, the C₃-C₆ cycloalkyl of the C₃-C₆ cycloalkyl fused to a C₆-C₁₀ aryl is substituted with C₁-C₃ alkyl, alkoxy, or halogen. In some embodiments, the C₃-C₆ cycloalkyl of the C₃-C₆ cycloalkyl fused to a phenyl is substituted with C₁-C₃ alkyl, alkoxy, or halogen.

In some embodiments of Formula (I), R⁶ and R¹, together with the carbon to which they are attached, form a C₃-C₆ cycloalkyl fused to a 5-6-membered heteroaryl. In some embodiments of Formula (I), the C₃-C₆ cycloalkyl of the C₃-C₆ cycloalkyl fused to a 5-6-membered heteroaryl is unsubstituted. In some embodiments of Formula (I), the C₃-C₆ cycloalkyl of the C₃-C₆ cycloalkyl fused to a 5-6-membered heteroaryl is substituted with C₁-C₃ alkyl, alkoxy, or halogen.

In some embodiments of Formula (I), R⁶ and R¹, together with the carbon to which they are attached, form a C₅ cycloalkyl fused to a 5-6-membered heteroaryl. In some embodiments of Formula (I), the C₅ cycloalkyl of the C₅ cycloalkyl fused to a 5-6-membered heteroaryl is unsubstituted. In some embodiments of Formula (I), the C₅ cycloalkyl of the C₅ cycloalkyl fused to a 5-6-membered heteroaryl is substituted with C₁-C₃ alkyl, alkoxy, or halogen.

In some embodiments of Formula (I), R⁶ and R¹, together with the carbon to which they are attached, form a C₅ cycloalkyl fused to a substituted or unsubstituted 5-membered heteroaryl. In some embodiments of Formula (I), R⁶ and R¹, together with the carbon to which they are attached, form a C₅ cycloalkyl fused to a substituted or unsubstituted 6-membered heteroaryl. In some embodiments of Formula (I), the C₅ cycloalkyl of the C₅ cycloalkyl fused to a 5-membered heteroaryl is unsubstituted. In some embodiments of Formula (I), the C₅ cycloalkyl of the C₅ cycloalkyl fused to a 6-membered heteroaryl is unsubstituted. In some embodiments of Formula (I), the C₅ cycloalkyl of the C₅ cycloalkyl fused to a 5-membered heteroaryl is substituted with C₁-C₃ alkyl, alkoxy, or halogen. In some embodiments of Formula (I), the C₅ cycloalkyl of the C₅ cycloalkyl fused to a 6-membered heteroaryl is substituted with C₁-C₃ alkyl, alkoxy, or halogen.

In some embodiments, the compound of Formula (I) has Formula (I-B):

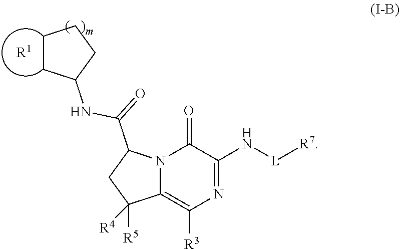

In Formula (I-B) m is 1 or 2. In some embodiments of Formula (I-B), m is 1. In some embodiments of Formula (I-B), m is 2.

In some embodiments of Formulae (I), (I-A), and (I-B), R¹ is unsubstituted C₆-C₁₀ aryl.

In some embodiments of Formulae (I), (I-A), and (I-B), R¹ is unsubstituted phenyl.

In some embodiments of Formulae (I), (I-A), and (I-B), R¹ is C₆-C₁₀ aryl substituted with one or more substituent, one to five substituents, one to four substituents, one to three substituents, one or two substituents, or one substituent.

In some embodiments of Formulae (I), (I-A), and (I-B), R¹ is C₆-C₁₀ aryl substituted with substituents selected from the group consisting of R^{1A}, R^{1B}, R^{1C}, R^{1D} and R^{1E}, wherein R^{1A}, R^{1B}, R^{1C}, R^{1D} and R^{1E} are each independently hydrogen, C(═NH)NH₂, C(═NH)NHC(═O)OR⁸, C(═NOC(═O)R⁸)NH₂, C(═NOC(═O)OR⁸)NH₂, C(═NOH)NH₂, C(═NH)NHC(═O)NHC(═O)N(CH₃)R¹³, C₁-C₆ alkyl, C₂-C₆ alkenyl, C₂-C₆ alkynyl, halogen, C₁-C₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃-C₁₀ cycloalkyl, and substituted or unsubstituted 4-10-membered heterocyclyl,
wherein R¹, R⁹, R¹⁰, R¹¹, and R¹², are, at each occurrence, independently selected from the group consisting of hydrogen, deuterium, C₁-C₆ alkyl, C₂-C₆ alkenyl, C₂-C₆ alkynyl, hydroxyl, C₁-C₆ alkoxy, aryl, arylalkyl, C₁-C₆ haloalkyl, C₁-C₆ haloalkoxy, C₁-C₆ hydroxyalkyl, C₃-C₁₀ cycloalkyl, heterocyclyl, heteroarylalkyl, and heteroaryl,
wherein R¹³ is a C₆-C₁₀ aryl or 5-10-membered heteroaryl, unsubstituted or substituted with CH₂OC(═O)CH₃,
wherein when any one of R^{1A}, R^{1B}, R^{1C}, R^{1D}, and R^{1E} is a substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃-C₁₀ cycloalkyl, or a substituted 4-10 membered heterocyclyl, the R^{1A}, R^{1B}, R^{1C}, R^{1D}, or R^{1E} is unsubstituted or substituted with one or more substituents selected from the group consisting of halo, CN, OR^{e}, SR^{e}, C(O)R^{e}, C(O)NR^{e}R^{f}, C(O)OR^{e}, OC(O)R^{e}, OC(O)NR^{e}R^{f}, NR^{e}R^{f}, NR^{e}C(O)R^{f}, NR^{e}C(O)NR^{f}R^{g}, NR^{e}C(O)OR^{f}, C(═NR^{e})NR^{f}R^{g}, NR^{e}C(═NR^{f})NR^{g}R^{h}, S(O)R^{e}, S(O)NR^{e}R^{f}, S(O)₂R^{e}, NR^{e}S(O)₂R^{f}, S(O)₂NR^{e}R^{f} and oxo, wherein R^{e}, R^{f}, R^{g}, and R^{h} are, at each occurrence, independently selected from the group consisting of hydrogen, C₁-C₆ alkyl, C₂-C₆ alkenyl, C₂-C₆ alkynyl, hydroxyl, C₁-C₆ alkoxy, aryl, arylalkyl, C₁-C₆ haloalkyl, C₁-C₆ haloalkoxy, C₁-C₆ hydroxyalkyl, C₃-C₁₀ cycloalkyl, heterocyclyl, and heteroaryl.

In some embodiments of Formulae (I), (I-A), and (I-B), R¹ is phenyl substituted with one or more substituent, one to five substituents, one to four substituents, one to three substituents, one or two substituents, or one substituent.

In some embodiments of Formulae (I), (I-A), and (I-B), R¹ is phenyl substituted with substituents selected from the group consisting of R^{1A}, R^{1B}, R^{1C}, R^{1D}, and R^{1E}, wherein R^{1A}, R^{1B}, R^{1C}, R^{1D} and R^{1E} are each independently hydrogen, C(═NH)NH₂, C(═NH)NHC(═O)OR⁸, C(═NOC(═O)R⁸)NH₂, C(═NOC(═O)OR⁸)NH₂, C(═NOH)NH₂, C(═NH)NHC(═O)NHC(═O)N(CH₃)R¹³, C₁-C₆ alkyl, C₂-C₆ alkenyl, C₂-C₆ alkynyl, halogen, C₁-C₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃-C₁₀ cycloalkyl, and substituted or unsubstituted 4-10-membered heterocyclyl,
wherein R⁸, R⁹, R¹⁰, R¹¹, and R¹², are, at each occurrence, independently selected from the group consisting of hydrogen, deuterium, C₁-C₆ alkyl, C₂-C₆ alkenyl, C₂-C₆ alkynyl, hydroxyl, C₁-C₆ alkoxy, aryl, arylalkyl, C₁-C₆ haloalkyl, C₁-C₆ haloalkoxy, C₁-C₆ hydroxyalkyl, C₃-C₁₀ cycloalkyl, heterocyclyl, heteroarylalkyl, and heteroaryl,
wherein R¹³ is a C₆-C₁₀ aryl or 5-10-membered heteroaryl, unsubstituted or substituted with CH₂OC(═O)CH₃,
wherein when any one of R^{1A}, R^{1B}, R^{1C}, R^{1D}, and R^{1E} is a substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃-C₁₀ cycloalkyl, or a substituted 4-10 membered heterocyclyl, the R^{1A}, R^{1B}, R^{1C}, R^{1D}, or R^{1E} is unsubstituted or substituted with one or more substituents selected from the group consisting of halo, CN, OR^{e}, SR^{e}, C(O)R^{e}, C(O)NR^{e}R^{f}, C(O)OR^{e}, OC(O)R^{e}, OC(O)NR^{e}R^{f}, NR^{e}R^{f}, NR^{e}C(O)R^{f}, NR^{e}C(O)NR^{f}R^{g}, NR^{e}C(O)OR^{f}, C(═NR^{e})NR^{f}R^{g}, NR^{e}C(═NR^{f})NR^{g}R^{h}, S(O)R^{e}, S(O)NR^{e}R^{f}, S(O)₂R^{e}, NR^{e}S(O)₂R^{f}, S(O)₂NR^{e}R^{f} and oxo, wherein R^{e}, R^{f}, R^{g}, and R^{h} are, at each occurrence, independently selected from the group consisting of hydrogen, C₁-C₆ alkyl, C₂-C₆ alkenyl, C₂-C₆ alkynyl, hydroxyl, C₁-C₆ alkoxy, aryl, arylalkyl, C₁-C₆ haloalkyl, C₁-C₆ haloalkoxy, C₁-C₆ hydroxyalkyl, C₃-C₁₀ cycloalkyl, heterocyclyl, and heteroaryl.

In some embodiments of Formulae (I), (I-A), and (I-B), R¹ is substituted with substituents independently selected from the group consisting of methyl, —NH₂, —CH(CH₃)NH₂, —CH₂NH₂, —C(═NH)NH₂, halogen, and tetrazolyl.

In some embodiments of Formulae (I), (I-A), and (I-B), R¹ is substituted with substituents independently selected from the group consisting of methyl, —NH₂, —CH(CH₃)NH₂, —CH₂NH₂, —C(═NH)NH₂, fluoro, chloro, and tetrazolyl.

In some embodiments of Formulae (I) and (I-A), R¹ is selected from the group consisting of:

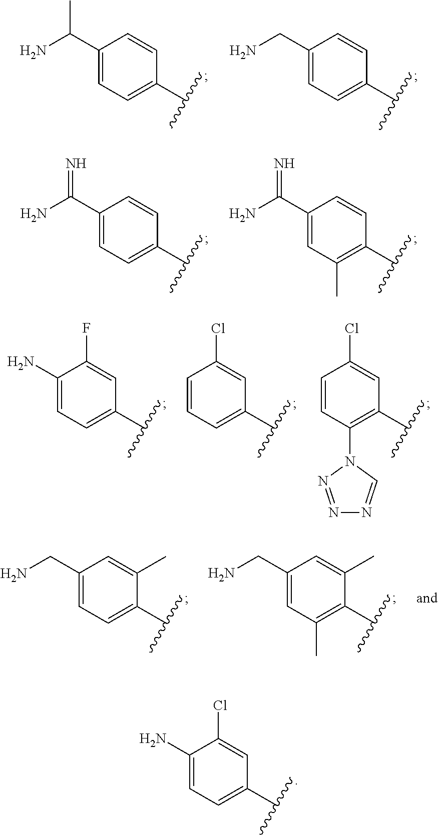

In some embodiments of Formula (I-B), R¹ is selected from the group consisting of:

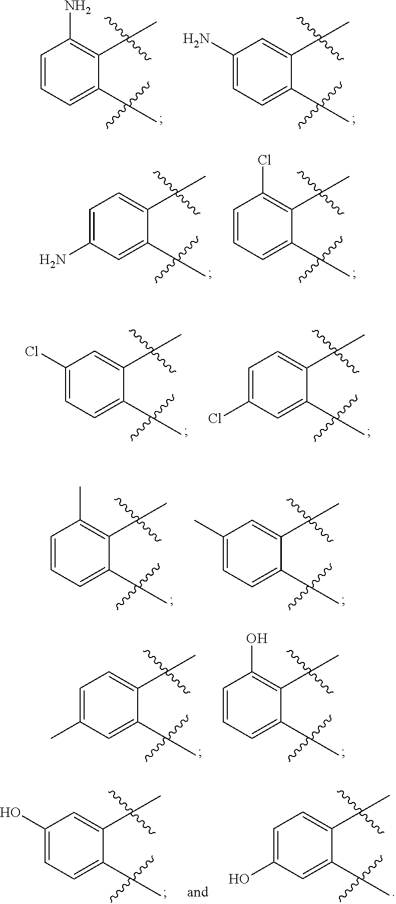

In some embodiments of Formula (I-B), R¹ is:

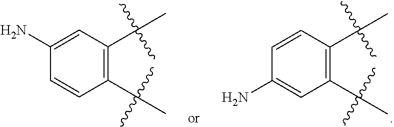 [SMILES: *c1ccc(N)cc1* ; *c1ccc(N)cc1*]

In some embodiments of Formulae (I), (I-A), and (I-B), R¹ is an unsubstituted 5-10-membered heteroaryl.

In some embodiments of Formulae (I), (I-A), and (I-B), R¹ is a 5-10-membered heteroaryl substituted with one or more substituent, one to five substituents, one to four substituents, one to three substituents, one or two substituents, or one substituent.

In some embodiments of Formulae (I), (I-A), and (I-B), R¹ is a 5-10-membered heteroaryl substituted with substituents selected from the group consisting of R^{1A}, R^{1B}, R^{1C}, R^{1D}, and R^{1E}, wherein R^{1A}, R^{1B}, R^{1C}, R^{1D}, and R^{1E} are each independently hydrogen, C(NH)NH₂, C(═NH)NHC(═O)OR⁸, C(═NOC(═O)R⁸)NH₂, C(═NOC(═O)OR⁸)NH₂, C(═NOH)NH₂, C(═NH)NHC(═O)NHC(═O)N(CH₃)R¹³, C₁-C₆ alkyl, C₂-C₆ alkenyl, C₂-C₆ alkynyl, halogen, C₁-C₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃-C₁₀ cycloalkyl, and substituted or unsubstituted 4-10 membered heterocyclyl, wherein the C₃-C₁₀ cycloalkyl and 4-10 membered heterocyclyl can be a fused, bridge, or spiro ring system,
wherein R⁸, R⁹, R¹⁰, R¹¹, and R¹², are, at each occurrence, independently selected from the group consisting of hydrogen, deuterium, C₁-C₆ alkyl, C₂-C₆ alkenyl, C₂-C₆ alkynyl, hydroxyl, C₁-C₆ alkoxy, aryl, arylalkyl, C₁-C₆ haloalkyl, C₁-C₆ haloalkoxy, C₁-C₆ hydroxyalkyl, C₃-C₁₀ cycloalkyl, heterocyclyl, heteroarylalkyl, and heteroaryl,
wherein R¹³ is a C₆-C₁₀ aryl or 5-10-membered heteroaryl, unsubstituted or substituted with CH₂OC(═O)CH₃,
wherein when any one of R^{1A}, R^{1B}, R^{1C}, R^{1D}, and R^{1E} is a substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃-C₁₀ cycloalkyl, or a substituted 4-10 membered heterocyclyl, the R^{1A}, R^{1B}, R^{1C}, R^{1D}, or R^{1E} is unsubstituted or substituted with one or more substituents selected from the group consisting of halo, CN, OR^{e}, SR^{e}, C(O)R^{e}, C(O)NR^{e}R^{f}, C(O)OR^{e}, OC(O)R^{e}, OC(O)NR^{e}R^{f}, NR^{e}R^{f}, NR^{e}C(O)R^{f}, NR^{e}C(O)NR^{f}R^{g}, NR^{e}C(O)OR^{f}, C(═NR^{e})NR^{f}R^{g}, NR^{e}C(═NR^{f})NR^{g}R^{h}, S(O)R^{e}, S(O)NR^{e}R^{f}, S(O)₂R^{e}, NR^{e}S(O)₂R^{f}, S(O)₂NR^{e}R^{f} and oxo, wherein R^{e}, R^{f}, R^{g}, and R^{h} are, at each occurrence, independently selected from the group consisting of hydrogen, C₁-C₆ alkyl, C₂-C₆ alkenyl, C₂-C₆ alkynyl, hydroxyl, C₁-C₆ alkoxy, aryl, arylalkyl, C₁-C₆ haloalkyl, C₁-C₆ haloalkoxy, C₁-C₆ hydroxyalkyl, C₃-C₁₀ cycloalkyl, heterocyclyl, and heteroaryl.

In some embodiments of Formulae (I), (I-A), and (I-B), the 5-10-membered heteroaryl of R¹ is selected from the group consisting of pyridinyl, dihydropyrrolopyridinyl, pyrrolopyridinyl, thiophenyl, imidazopyridinyl, benzoimidazolyl, dihydropyrrolothiophenyl, and isoquinoline.

In some embodiments of Formulae (I) and (I-A), R¹ has one of the structures:

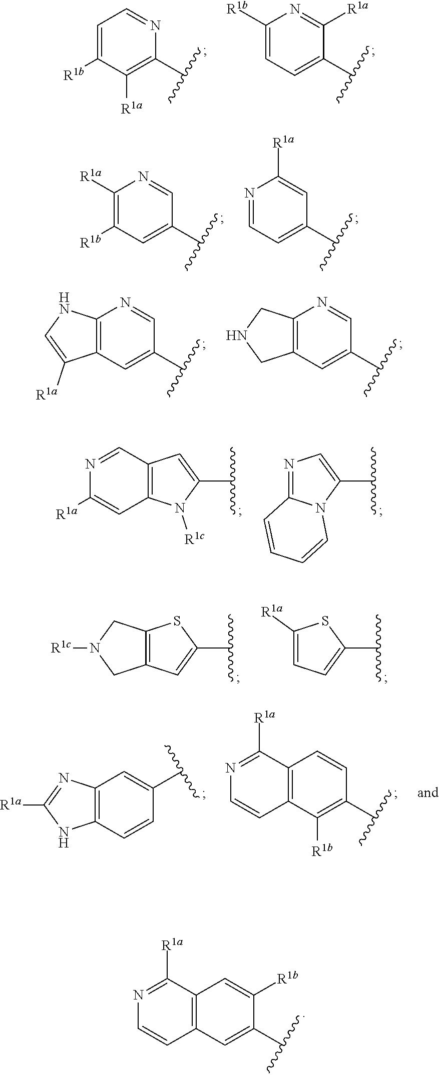

In some embodiments of Formulae (I) and (I-A), R^{1a} and Rib are each independently selected from the group consisting of hydrogen, C₁-C₆ alkyl, C₁-C₆ deuterated alkyl, amino, aminylalkyl, alkoxy, and halogen.

In some embodiments of Formulae (I) and (I-A), R^{1c} is hydrogen or C₁-C₆ alkyl.

In some embodiments of Formulae (I) and (I-A), the 5-10-membered heteroaryl of R¹ is substituted with at least one substituent, one or more substituent, one to five substituents, one to four substituents, one to three substituents, one or two substituents, or one substituent.

In some embodiments of Formulae (I) and (I-A), the 5-10-membered heteroaryl of R¹ is substituted with substituents independently selected from the group consisting of methyl, halogen, aminyl, —CH₂NH₂, pyrrolidinyl, N-methyl pyrrolidinyl, and methoxy. In some embodiments of Formulae (I) and (I-A), the 5-10-membered heteroaryl of R¹ is substituted with —CD₃. In some embodiments of Formulae (I) and (I-A), the 5-10-membered heteroaryl of R¹ is substituted with fluoro or chloro.

In some embodiments of Formulae (I) and (I-A), R¹ is selected from the group consisting of:

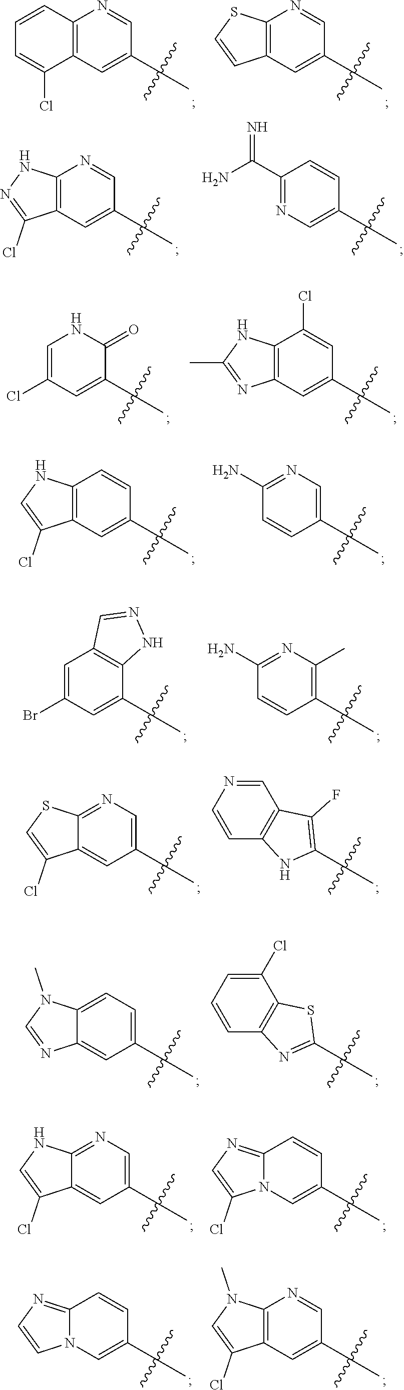

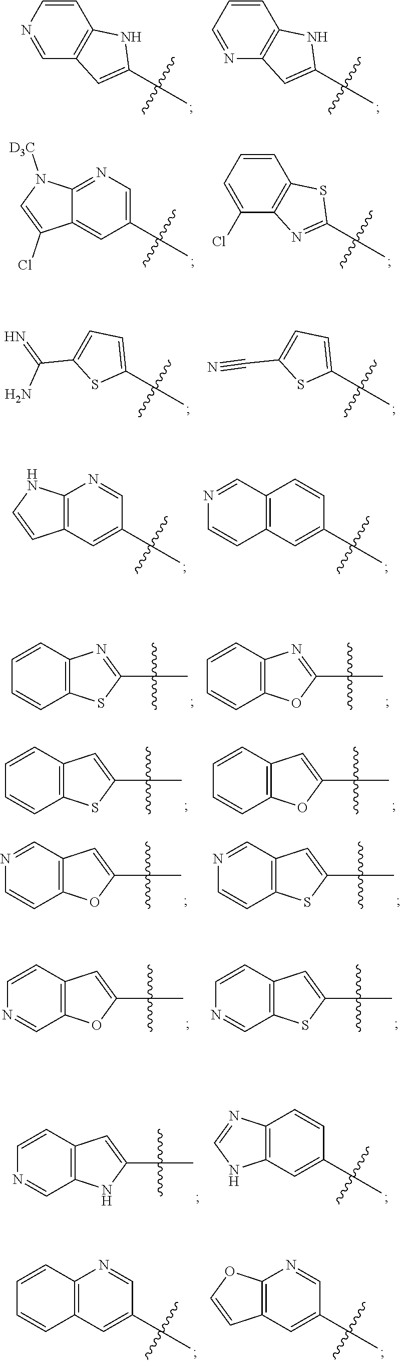

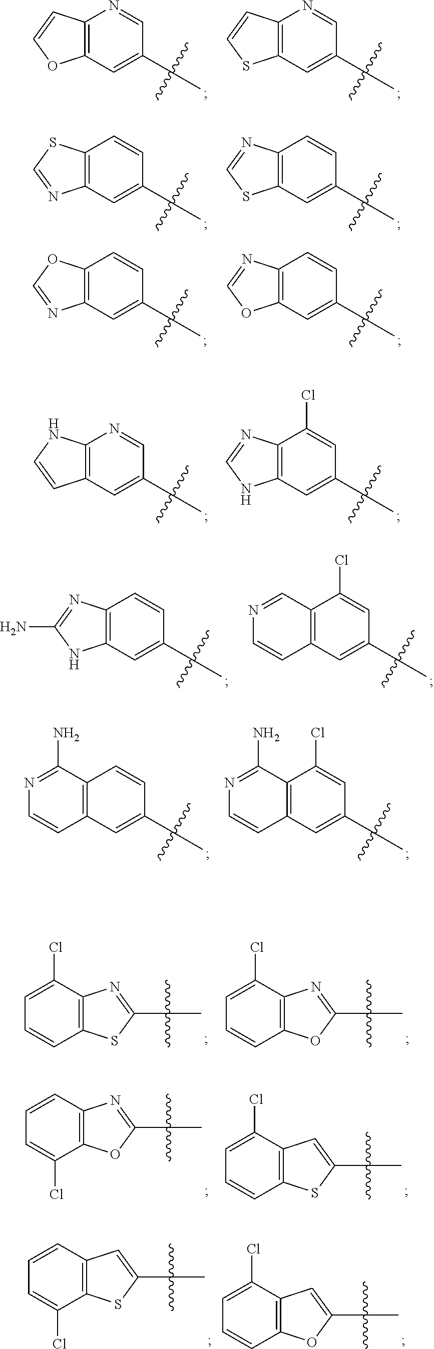

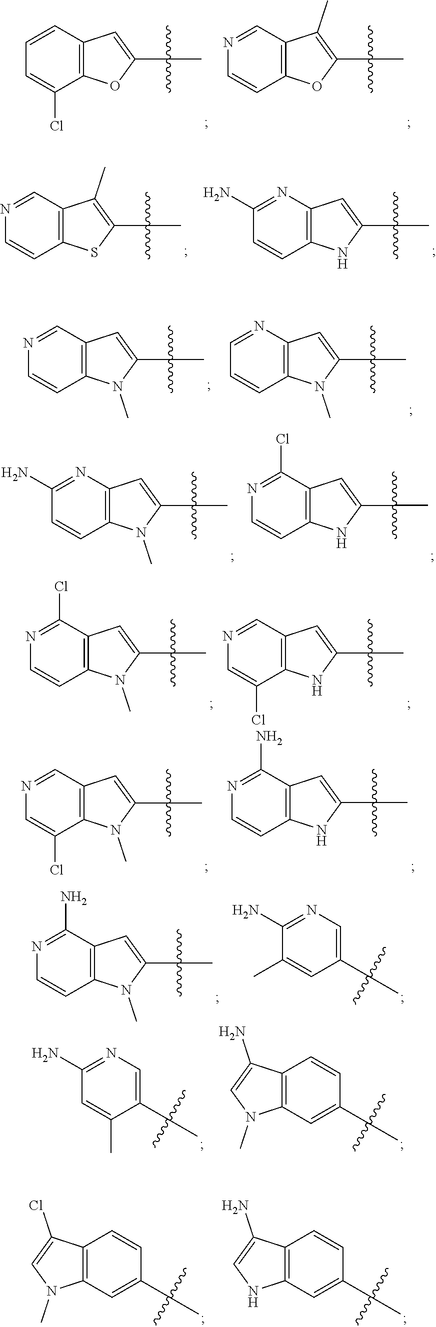

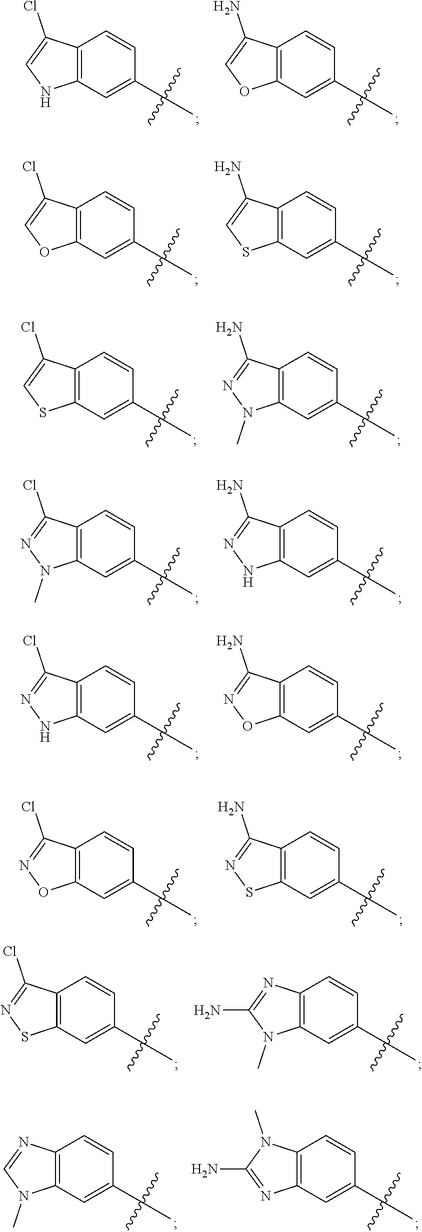

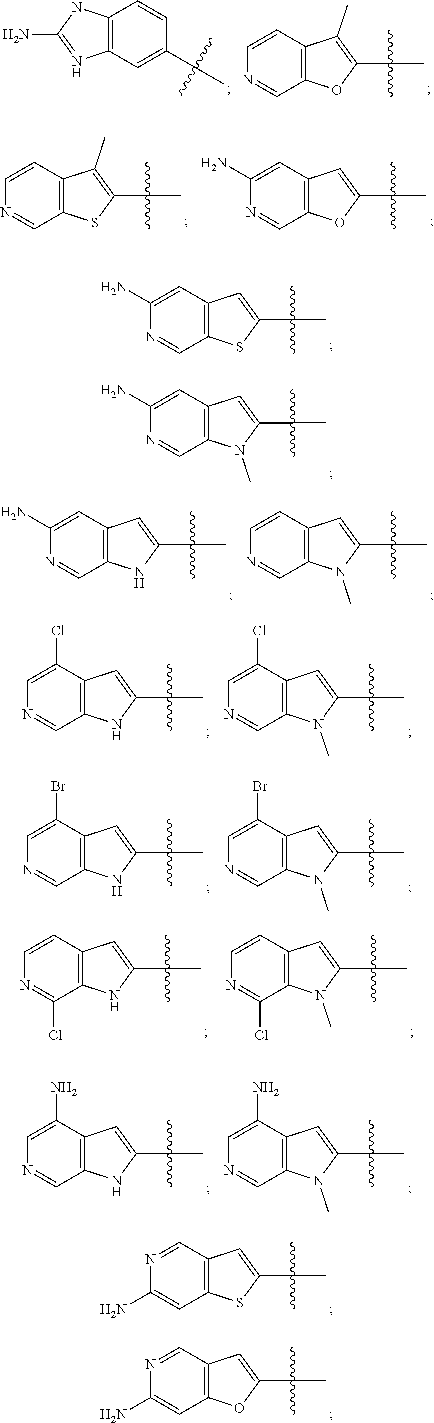

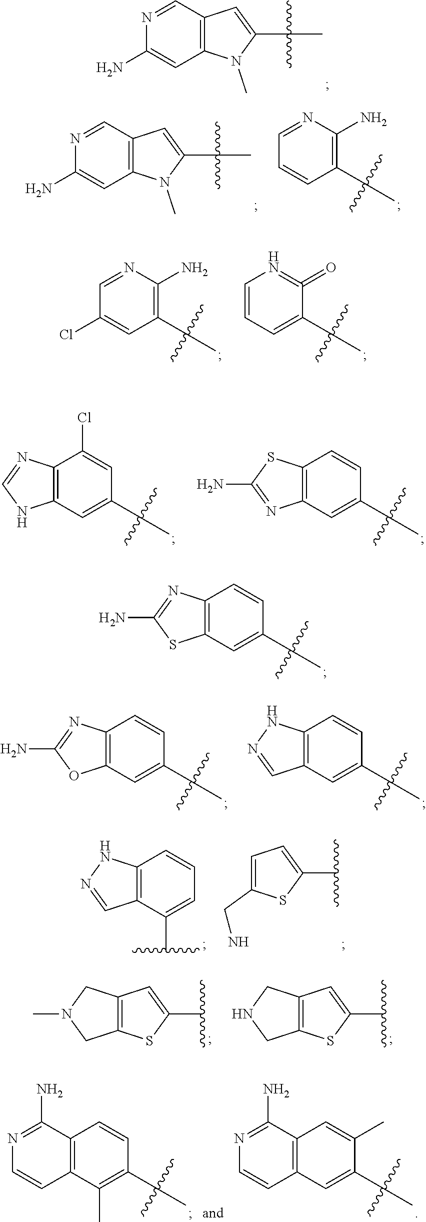

In some embodiments of Formulae (I) and (I-A), R¹ is selected from the group consisting of:

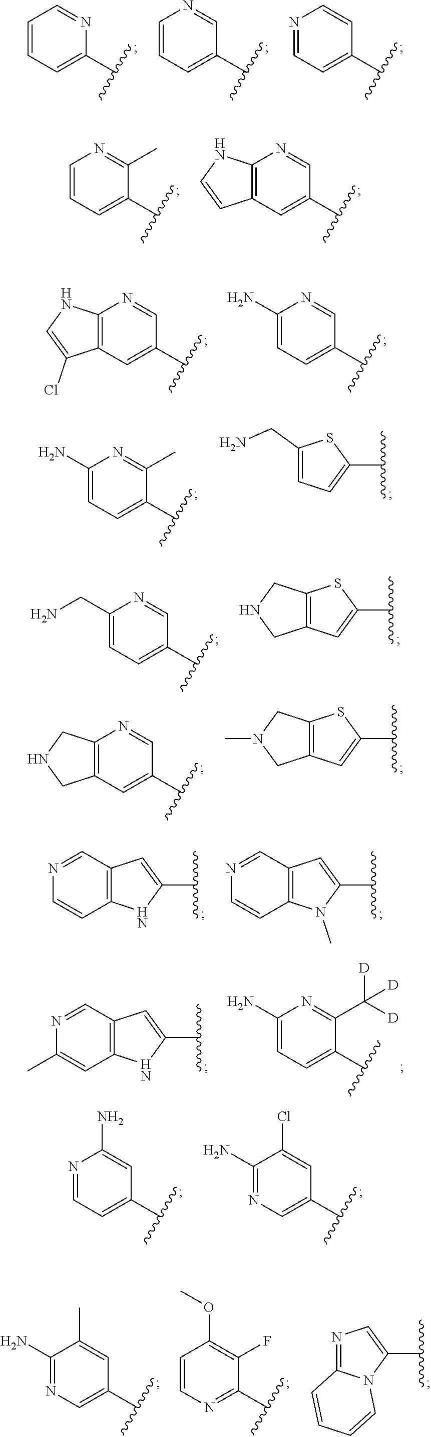

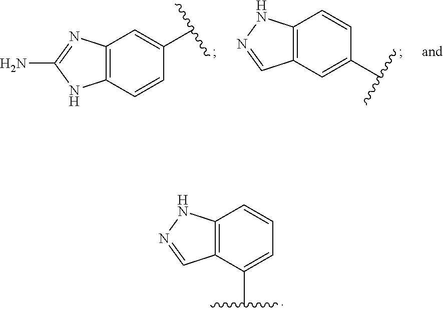 [SMILES: *c1ccc2[nH]c(N)nc2c1 ; *c1ccc2[nH]ncc2c1 ; *c1cccc2[nH]ncc12]

In some embodiments of Formula (I-B), R¹ is selected from the group consisting of:

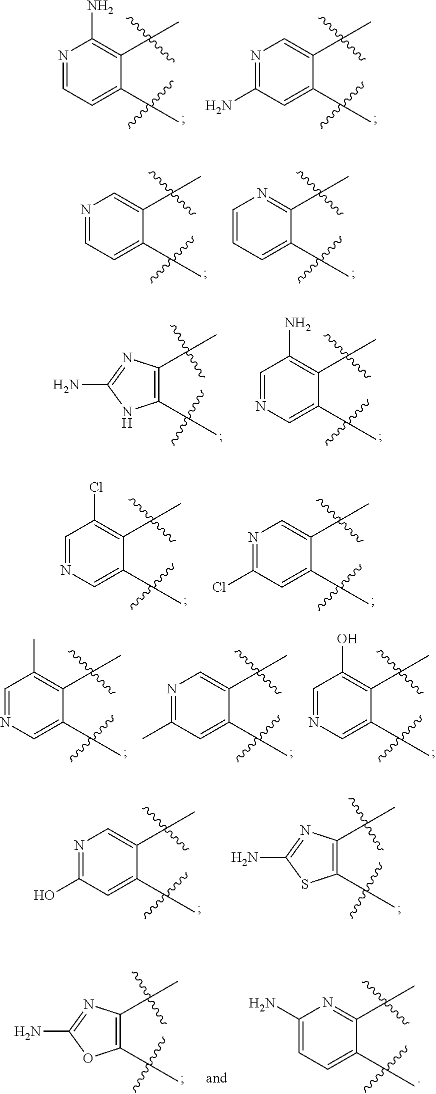

In some embodiments of Formula (I-B), R¹ has one of the structures:

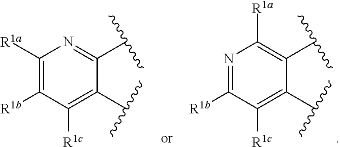

In some embodiments of Formula (I-B), R^{1a}, R^{1b}, and R^{1c} are each independently selected from the group consisting of hydrogen, C₁-C₆ alkyl, C₁-C₆ deuterated alkyl, amino, aminylalkyl, alkoxy, and halogen, and wherein R^{1d} is selected from the group consisting of hydrogen and C₁-C₆ alkyl.

In some embodiments of Formula (I-B), R¹ is

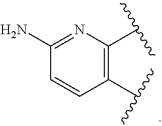 [SMILES: *c1ccc(N)nc1*]

In some aspects, the present disclosure provides a compound having Formula (II):

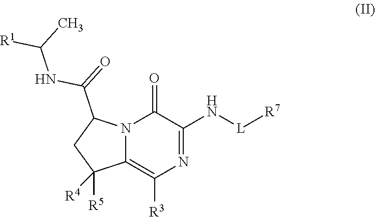

wherein:
R¹ is a substituted or unsubstituted 5-10-membered heteroaryl consisting of a 5-10-membered monocyclic or bicyclic ring system comprising one to eight carbon atoms, one to six heteroatoms selected from the group consisting of nitrogen, oxygen, and sulfur, and at least one aromatic ring, and which can comprise a fused or bridged ring system.

In some embodiments of Formula (II), at least one of R³, R⁴, and R⁵ is not hydrogen, and R³ is not Cl when R¹ is

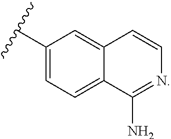 [SMILES: *c1ccc2c(N)nccc2c1]

In some embodiments of Formula (II), R¹ is an unsubstituted 5-10-membered heteroaryl.

In some embodiments of Formula (II), R¹ is a 5-10-membered heteroaryl substituted with one or more substituent, one to five substituents, one to four substituents, one to three substituents, one or two substituents, or one substituent.

In some embodiments of Formula (II), R¹ is a 5-10-membered heteroaryl substituted with substituents selected from the group consisting of R^{1A}, R^{1B}, R^{1C}, R^{1D} and R^{1E}, wherein R^{1A}, R^{1B}, R^{1C}, R^{1D}, and R^{1E} are each independently hydrogen, C(NH)NH₂, C(═NH)NHC(═O)OR⁸, C(═NOC(═O)R⁸)NH₂, C(═NOC(═O)OR⁸)NH₂, C(═NOH)NH₂, C(═NH)NHC(═O)NHC(═O)N(CH₃)R¹³, C₁-C₆ alkyl, C₂-C₆ alkenyl, C₂-C₆ alkynyl, halogen, C₁-C₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, wherein the C₃₋₁₀ cycloalkyl and 4-10 membered heterocyclyl can be a fused, bridge, or spiro ring system,
wherein R⁸, R⁹, R¹⁰, R¹¹, and R¹², are, at each occurrence, independently selected from the group consisting of hydrogen, deuterium, C₁-C₆ alkyl, C₂-C₆ alkenyl, C₂-C₆ alkynyl, hydroxyl, C₁-C₆ alkoxy, aryl, arylalkyl, C₁-C₆ haloalkyl, C₁-C₆ haloalkoxy, C₁-C₆ hydroxyalkyl, cycloalkyl, heterocyclyl, heteroarylalkyl, and heteroaryl,
wherein R¹³ is a C₆-C₁₀ aryl or 5-10-membered heteroaryl, unsubstituted or substituted with CH₂OC(═O)CH₃,
wherein when any one of R^{1A}, R^{1B}, R^{1C}, R^{1D}, or R^{1E} is a substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl, the R^{1A}, R^{1B}, R^{1C}, R^{1D}, or R^{1E} is unsubstituted or substituted with one or more substituents selected from the group consisting of halo, CN, OR^{e}, SR^{e}, C(O)R^{e}, C(O)NR^{e}R^{f}, C(O)OR^{e}, OC(O)R^{e}, OC(O)NR^{e}R^{f}, NR^{e}R^{f}, NR^{e}C(O)R^{f}, NR^{e}C(O)NR^{f}R^{g}, NR^{e}C(O)OR^{f}, C(═NR^{e})NR^{f}R^{g}, NR^{e}C(═NR^{f})NR^{g}R^{h}, S(O)R^{e}, S(O)NR^{e}R^{f}, S(O)₂R^{e}, NR^{e}S(O)₂R^{f}, S(O)₂NR^{e}R^{f} and oxo, wherein R^{e}, R^{f}, R^{g}, and R^{h} are, at each occurrence, independently selected from the group consisting of hydrogen, C₁-C₆ alkyl, C₂-C₆ alkenyl, C₂-C₆ alkynyl, hydroxyl, C₁-C₆ alkoxy, aryl, arylalkyl, C₁-C₆ haloalkyl, C₁-C₆ haloalkoxy, C₁-C₆ hydroxyalkyl, cycloalkyl, heterocyclyl, and heteroaryl.

In some embodiments of Formula (II), R¹ is a substituted 5-10-membered heteroaryl substituted with substituents selected from the group consisting of hydrogen, C₁-C₆ alkyl, C₁-C₆ deuterated alkyl, amino, aminylalkyl, alkoxy, and halogen.

In some embodiments of Formula (II), the 5-10-membered heteroaryl of R¹ is selected from the group consisting of pyridinyl, dihydropyrrolopyridinyl, pyrrolopyridinyl, thiophenyl, imidazopyridinyl, and dihydropyrrolothiophenyl.

In some embodiments of Formula (II), R¹ is selected from the group consisting of:

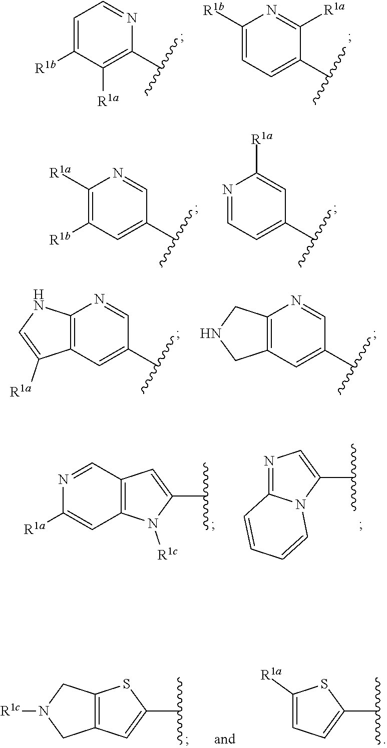

In some embodiments of Formula (II), R^{1a} and R^{1b} are each independently selected from the group consisting of hydrogen, C₁-C₆ alkyl, C₁-C₆ deuterated alkyl, amino, aminylalkyl, alkoxy, and halogen. In some embodiments of Formula (II), R^{1c} is hydrogen or C₁-C₆ alkyl.

In some embodiments of Formula (II), R¹ is selected from the group consisting of:

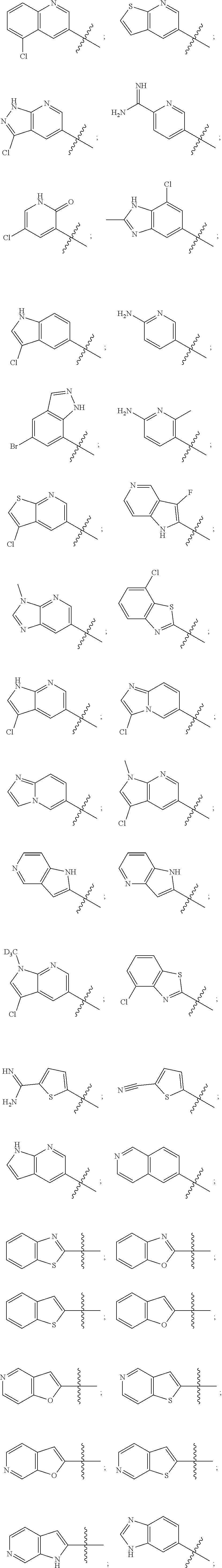

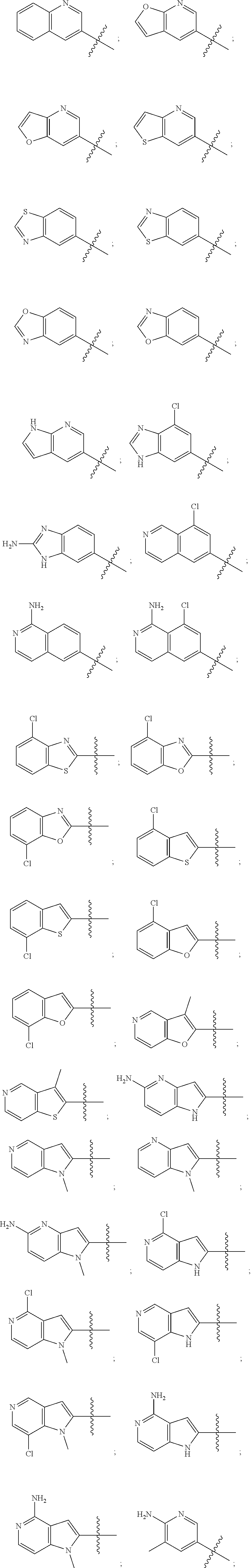

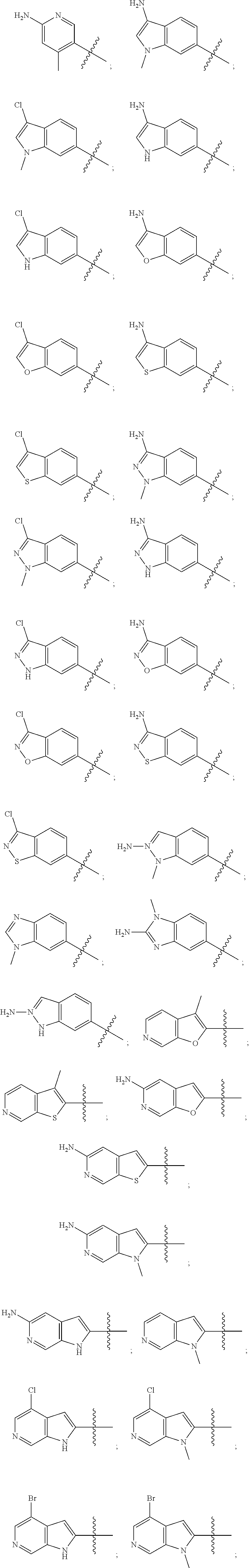

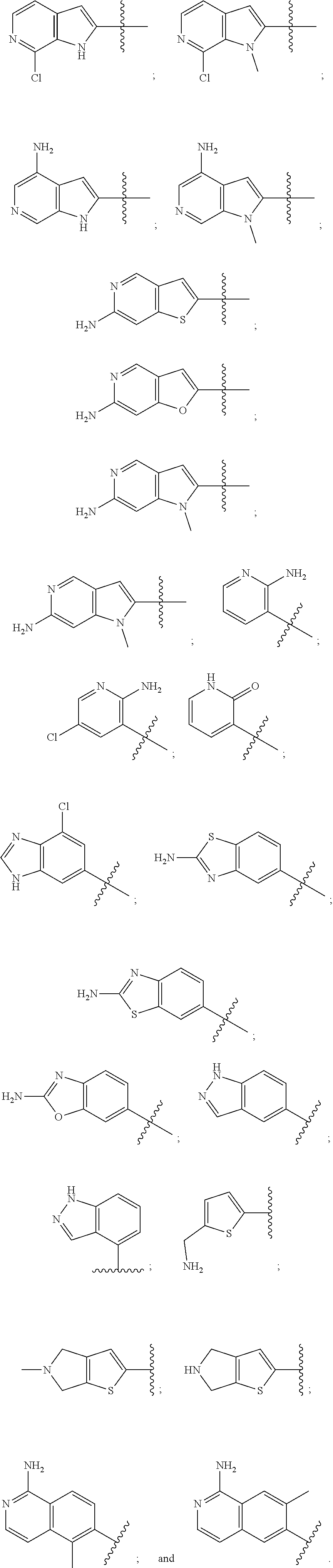

In some embodiments of Formula (II), R¹ is selected from the group consisting of:

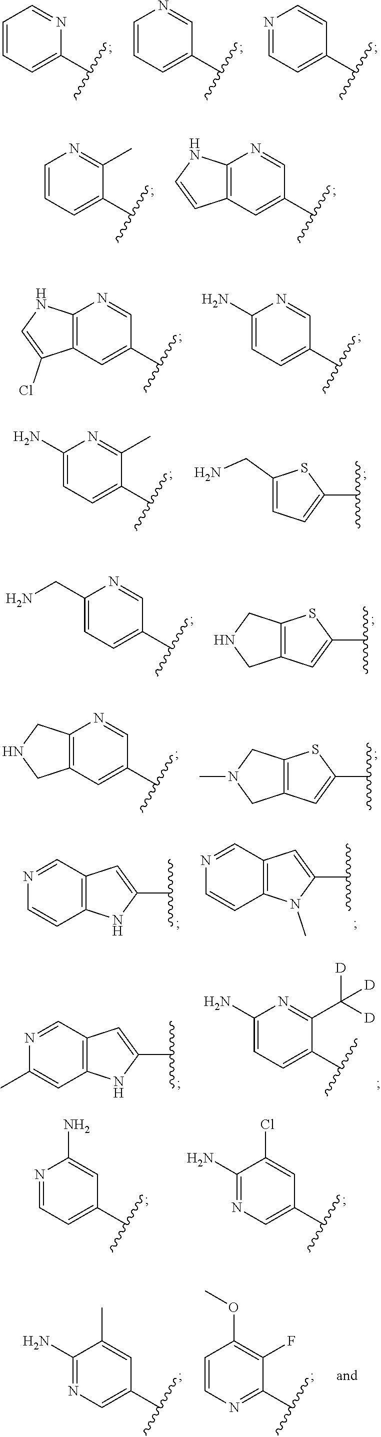

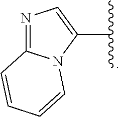 [SMILES: *c1cnc2ccccn12]

In some embodiments of Formula (II), R¹ is selected from the group consisting of:

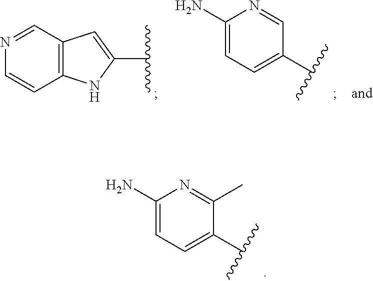

In some aspects, the present disclosure provides a compound having Formula (III):

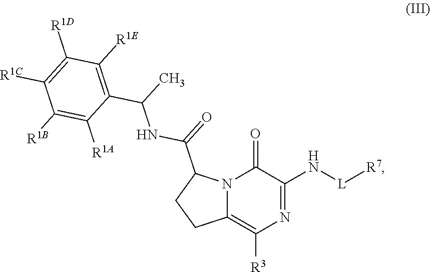

wherein:
R^{1A}, R^{1B}, R^{1C}, R^{1D}, and R^{1E} are each independently selected from the group consisting of hydrogen, C(NH)NH₂, C(═NH)NHC(═O)OR′, C(═NOC(═O)R⁸)NH₂, C(═NOC(═O)OR⁸)NH₂, C(═NOH)NH₂, C(═NH)NHC(═O)NHC(═O)N(CH₃)R¹³, C₁-C₆ alkyl, C₂-C₆ alkenyl, C₂-C₆ alkynyl, halogen, C₁-C₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, wherein the C₃₋₁₀ cycloalkyl and 4-10 membered heterocyclyl can be a fused, bridge, or spiro ring system,
wherein R⁸, R⁹, R¹⁰, R¹¹, and R¹², are, at each occurrence, independently selected from the group consisting of hydrogen, deuterium, C₁-C₆ alkyl, C₂-C₆ alkenyl, C₂-C₆ alkynyl, hydroxyl, C₁-C₆ alkoxy, aryl, arylalkyl, C₁-C₆ haloalkyl, C₁-C₆ haloalkoxy, C₁-C₆ hydroxyalkyl, cycloalkyl, heterocyclyl, heteroarylalkyl, and heteroaryl,
wherein R¹³ is a C₆-C₁₀ aryl or 5-10-membered heteroaryl, optionally substituted with CH₂OC(═O)CH₃,
wherein when any one of R^{1A}, R^{1B}, R^{1C}, R^{1D}, or R^{1E} is a substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl, the R^{1A}, R^{1B}, R¹ⁱ, R^{1D}, or R^{1E} is unsubstituted or substituted with one or more substituents selected from the group consisting of halo, CN, OR^{e}, SR^{e}, C(O)R^{e}, C(O)NR^{e}R^{f}, C(O)OR^{e}, OC(O)R^{e}, OC(O)NR^{e}R^{f}, NR^{e}R^{f}, NR^{e}C(O)R^{f}, NR^{e}C(O)NR^{f}R^{g}, NR^{e}C(O)OR^{f}, C(═NR^{e})NR^{f}R^{g}, NR^{e}C(═NR^{f})NR^{g}R^{h}, S(O)R^{e}, S(O)NR^{e}R^{f}, S(O)₂R^{e}, NR^{e}S(O)₂R^{f}, S(O)₂NR^{e}R^{f} and oxo, wherein R^{e}, R^{f}, R^{g}, and R^{h} are, at each occurrence, independently selected from the group consisting of hydrogen, C₁-C₆ alkyl, C₂-C₆ alkenyl, C₂-C₆ alkynyl, hydroxyl, C₁-C₆ alkoxy, aryl, arylalkyl, C₁-C₆ haloalkyl, C₁-C₆ haloalkoxy, C₁-C₆ hydroxyalkyl, cycloalkyl, heterocyclyl, and heteroaryl.

In some embodiments of Formula (III), R^{1A}, R^{1B}, R^{1C}, R^{1D}, and R^{1E} are each independently selected from the group consisting of hydrogen, linear or branched C₁-C₃ alkyl, linear or branched C₁-C₃ aminylalkyl, halogen, tetrazolyl, and amidinyl.

In some embodiments of Formula (III), R^{1A}, R^{1B}, R^{1C}, R^{1D}, and R^{1E} are each independently selected from the group consisting of hydrogen, methyl, CH(CH₃)NH₂, CH₂NH₂, C(═NH)NH₂, fluoro, chloro, and tetrazolyl.

In some embodiments of Formula (III), at least one of R^{1A}, R^{1B}, R^{1C}, R^{1D}, and R^{1E} is not hydrogen.

In some aspects, the present disclosure provides a compound having Formula (IV):

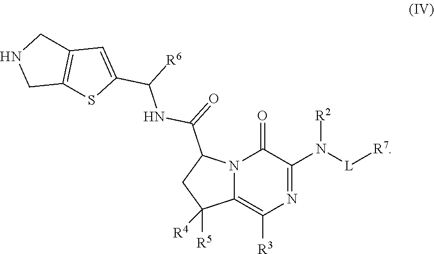

In some embodiments of Formula (IV), R⁶ is hydrogen. In some embodiments of Formula (IV), R⁶ is methyl.

In some embodiments of Formulae (I) and (IV), R² is hydrogen. In some embodiments of Formula (I) and (IV), R² is methyl.

In some embodiments of Formulae (I), (I-A), (I-B), (II), (III), and (IV), R³ is hydrogen. In some embodiments of Formulae (I), (I-A), (I-B), (II), (III), and (IV), R³ is substituted or unsubstituted C₁-C₃ alkyl. In some embodiments of Formulae (I), (I-A), (I-B), (II), (III), and (IV), R³ is methyl, ethyl, n-propyl, or isopropyl. In some embodiments of Formulae (I), (I-A), (I-B), (II), (III), and (IV), R³ is substituted or unsubstituted C₃-C₆ cycloalkyl. In some embodiments of Formulae (I), (I-A), (I-B), (II), (III), and (IV), R³ is substituted or unsubstituted 5-6-membered saturated or unsaturated heterocyclyl. In some embodiments of Formulae (I), (I-A), (I-B), (II), (III), and (IV), R³ is dihydropyran. In some embodiments of Formulae (I), (I-A), (I-B), (II), (III), and (IV), R³ is substituted or unsubstituted phenyl. In some embodiments of Formulae (I), (I-A), (I-B), (II), (III), and (IV), R³ is substituted or unsubstituted 5-6-membered heteroaryl. In some embodiments of Formulae (I), (I-A), (I-B), (II), (III), and (IV), R³ is pyrazolyl. In some embodiments of Formulae (I), (I-A), (I-B), (II), (III), and (IV), R³ is halogen. In some embodiments of Formulae (I), (I-A), (I-B), (II), (III), and (IV), R³ is chloro. In some embodiments of Formulae (I), (I-A), (I-B), (II), (III), and (IV), R³ is cyano. In some embodiments of Formulae (I), (I-A), (I-B), (II), (III), and (IV), R³ is CD₃.

In some embodiments of Formulae (I), (I-A), (I-B), (II), (III), and (IV), the substituted C₁-C₃ alkyl, substituted C₃-C₆ cycloalkyl, substituted 5-6-membered saturated or unsaturated heterocyclyl, substituted phenyl, and substituted 5-6-membered heteroaryl of R³ is substituted with one or more substituent, one to five substituents, one to four substituents, one to three substituents, one or two substituents, or one substituent.

In some embodiments of Formulae (I), (I-A), (I-B), (II), (III), and (IV), the substituted C₁-C₃ alkyl, substituted C₃-C₆ cycloalkyl, substituted 5-6-membered saturated or unsaturated heterocyclyl, substituted phenyl, and substituted 5-6-membered heteroaryl of R³ is substituted with substituents selected from the group consisting of C₁-C₃ alkyl, halo, or haloalkyl.

In some embodiments of Formulae (I), (I-A), (I-B), (II), (III), and (IV), the substituted C₁-C₃ alkyl, substituted C₃-C₆ cycloalkyl, substituted 5-6-membered saturated or unsaturated heterocyclyl, substituted phenyl, and substituted 5-6-membered heteroaryl of R³ is substituted with substituents selected from the group consisting of methyl, ethyl, and propyl. In some embodiments of Formulae (I), (I-A), (I-B), (II), (III), and (IV), the substituted C₁-C₃ alkyl, substituted C₃-C₆ cycloalkyl, substituted 5-6-membered saturated or unsaturated heterocyclyl, substituted phenyl, and substituted 5-6-membered heteroaryl of R³ is substituted with substituents selected from the group consisting of fluoroalkyl, difluoroalkyl, and trifluoroalkyl. In some embodiments of Formulae (I), (I-A), (I-B), (II), (III), and (IV), the substituted C₁-C₃ alkyl, substituted C₃-C₆ cycloalkyl, substituted 5-6-membered saturated or unsaturated heterocyclyl, substituted phenyl, and substituted 5-6-membered heteroaryl of R³ is substituted with halogen.

In some embodiments of Formulae (I), (I-A), (I-B), (II), (III), and (IV), the substituted phenyl of R³ is substituted with one or more substituents independently selected from the group consisting of methyl, ethyl, propyl, fluoroalkyl, difluoroalkyl, and trifluoroalkyl.

In some embodiments of Formulae (I), (I-A), (I-B), (II), (III), and (IV), the substituted 5-6-membered heteroaryl of R³ is substituted with one or more substituents independently selected from the group consisting of methyl, ethyl, propyl, fluoroalkyl, difluoroalkyl, and trifluoroalkyl.

In some embodiments of Formulae (I), (I-A), (I-B), (II), (III), and (IV), the substituted phenyl and substituted 5-6-membered heteroaryl of R³ are substituted with one substituent selected from the group consisting of methyl, ethyl, propyl, fluoroalkyl, difluoroalkyl, and trifluoroalkyl.

In some embodiments of Formulae (I), (I-A), (I-B), (II), (III), and (IV), when R³ comprises one or more chiral center, each chiral center has (R) stereochemistry, (S) stereochemistry, or is a mixture thereof. For example, when R³ is substituted C₃-C₆ cycloalkyl and a comprises one or more chiral center, each chiral center of R³ has (R) stereochemistry, (S) stereochemistry, or is a mixture thereof.

In some embodiments of Formula (II), R³ is selected from the group consisting of hydrogen, substituted or unsubstituted C₁-C₃ alkyl, substituted or unsubstituted C₃-C₆ cycloalkyl, substituted or unsubstituted phenyl, substituted or unsubstituted 5-6-membered heteroaryl, halogen, cyano, and CD₃, wherein the substituted C₁-C₃ alkyl, C₃-C₆ cycloalkyl, phenyl, and 5-6-membered heteroaryl of R³ are substituted with methyl, fluoroalkyl, difluoroalkyl, or trifluoroalkyl.

In some embodiments of Formulae (I), (I-A), (I-B), (II), (III), and (IV), R³ is selected from the group consisting of:

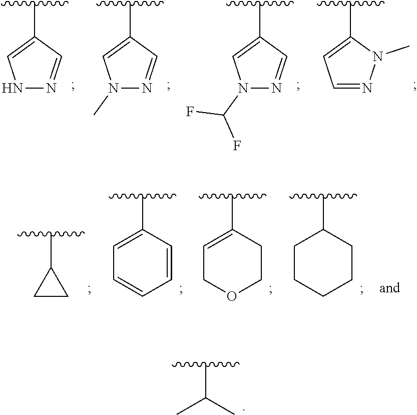

In some embodiments of Formulae (I), (I-A), (I-B), (II), and (IV), R⁴ and R⁵ are each independently selected from the group consisting of hydrogen, substituted or unsubstituted C₁-C₆ alkyl, and substituted or unsubstituted arylalkyl.

In some embodiments of Formulae (I), (I-A), (I-B), (II), and (IV), R⁴ and R⁵ are each independently selected from the group consisting of hydrogen, substituted or unsubstituted C₁-C₆ alkyl, and substituted or unsubstituted benzyl.

In some embodiments of Formulae (I), (I-A), (I-B), (II), and (IV), R⁴ and R⁵ are each independently selected from the group consisting of hydrogen, substituted or unsubstituted C₁-C₂ alkyl, and substituted or unsubstituted benzyl.

In some embodiments of Formulae (I), (I-A), (I-B), (II), and (IV), R⁴ is selected from the group consisting of hydrogen, C₁-C₆ alkyl, and benzyl. In some embodiments of Formulae (I), (I-A), (I-B), (II), (III), and (IV), R⁴ is hydrogen. In some embodiments of Formulae (I), (I-A), (I-B), (II), (III), and (IV), R⁵ is hydrogen. In some embodiments of Formulae (I), (I-A), (I-B), (II), (III), and (IV), R⁵ is methyl.

In some embodiments of Formulae (I), (I-A), (I-B), (II), and (IV), the substituted C₁-C₆ alkyl and substituted C₁-C₂ alkyl of R⁴ and R⁵ is substituted with one or more of C₁-C₃ alkyl, C₁-C₃ alkoxy, and halogen. In some embodiments of Formulae (I), (I-A), (I-B), (II), and (IV), the substituted benzyl of R⁴ and R⁵ is substituted with one or more of C₁-C₃ alkyl, C₁-C₃ alkoxy, and halogen.

In some embodiments of Formulae (I), (I-A), (I-B), (II), and (IV), R⁴ and R⁵, together with the carbon to which they are attached, form a substituted or unsubstituted C₃-C₆ cycloalkyl. In some embodiments of Formulae (I), (I-A), (I-B), (II), and (IV), R⁴ and R⁵, together with the carbon to which they are attached, form a substituted or unsubstituted C₃-C₅ cycloalkyl. In some embodiments of Formulae (I), (I-A), (I-B), (II), and (IV), R⁴ and R⁵, together with the carbon to which they are attached, form a substituted or unsubstituted 5-6-membered cycloalkenyl. In some embodiments of Formulae (I), (I-A), (I-B), (II), and (IV), R⁴ and R⁵, together with the carbon to which they are attached, form a substituted or unsubstituted 5-membered cycloalkenyl. In some embodiments of Formulae (I), (I-A), (I-B), (II), and (IV), R⁴ and R⁵, together with the carbon to which they are attached, form a substituted or unsubstituted 6-membered cycloalkenyl.

In some embodiments of Formulae (I), (I-A), (I-B), (II), and (IV), the substituted C₃-C₆ cycloalkyl, substituted C₃-C₅ cycloalkyl, substituted 5-6-membered cycloalkenyl, substituted 5-membered cycloalkenyl, and substituted 6-membered cycloalkenyl of R⁴ and R⁵, together with the carbon to which they are attached, are substituted with one or more of C₁-C₃ alkyl, C₁-C₃ alkoxy, and halogen.

In some embodiments of Formulae (I), (I-A), (I-B), (II), and (IV), R⁴ and R⁵, together with the carbon to which they are attached, form a substituted or unsubstituted C₃-C₅ cycloalkyl, wherein the substituted C₃-C₅ cycloalkyl is substituted with hydroxy, alkoxy, amino, halo, cyano, alkyl, alkylsulfonyl, or a combination thereof.

In some embodiments of Formula (II), R⁴ is hydrogen, C₁-C₆ alkyl, or benzyl, and R⁵ is hydrogen or methyl. In some embodiments of Formula (II), R⁴ and R⁵, together with the carbon to which they are attached, form a C₃-C₅ cycloalkyl or a 6-membered cycloalkenyl.

In some embodiments of Formulae (I), (I-A), (I-B), (II), and (IV), when a chiral center is present at the carbon atom to which R⁴ and R⁵ are attached, the compound comprises (R) stereochemistry, (S) stereochemistry, or is a mixture thereof. In some embodiments of Formulae (I), (I-A), (I-B), (II), and (IV), when a chiral center is present in R⁴ and/or R⁵, each chiral center comprises (R) stereochemistry, (S) stereochemistry, or is a mixture thereof.

In some embodiments of Formulae (I), (I-A), (I-B), (II), (III), and (IV), R⁷ is selected from the group consisting of hydrogen, substituted or unsubstituted C₁-C₃ alkyl, substituted or unsubstituted 5-6-membered heteroalkyl, substituted or unsubstituted C₃-C₆ cycloalkyl, substituted or unsubstituted C₃-C₆ cycloalkyl fused with aryl, substituted or unsubstituted 3-6-membered heterocyclyl, substituted or unsubstituted C₆-C₁₀ aryl, substituted or unsubstituted C₆-C₁₀ aryl fused with C₅-C₆ cycloalkyl, substituted or unsubstituted 5-9-membered heteroaryl, substituted or unsubstituted 5-9-membered heteroaryl fused with C₅-C₆ cycloalkyl, alkoxy, and halogen.

In some embodiments, R⁷ is hydrogen. In some embodiments, when R⁷ is hydrogen, n is 0. In some embodiments, R⁷ is substituted or unsubstituted C₁-C₃ alkyl. In some embodiments, R⁷ is substituted or unsubstituted 5-6-membered heteroalkyl. In some embodiments, R⁷ is substituted or unsubstituted 6-membered heteroalkyl. In some embodiments, R⁷ is substituted or unsubstituted C₃-C₆ cycloalkyl. In some embodiments, R⁷ is substituted or unsubstituted C₃-C₆ cycloalkyl fused with aryl. In some embodiments, R⁷ is substituted or unsubstituted C₃-C₆ cycloalkyl fused with phenyl. In some embodiments, R⁷ is substituted or unsubstituted 3-6-membered heterocyclyl. In some embodiments, R⁷ is substituted or unsubstituted C₆-C₁₀ aryl. In some embodiments, R⁷ is substituted or unsubstituted phenyl. In some embodiments, R⁷ is substituted or unsubstituted C₆-C₁₀ aryl fused with C₅-C₆ cycloalkyl. In some embodiments, R⁷ is substituted or unsubstituted phenyl fused with C₆ cycloalkyl. In some embodiments, R⁷ is substituted or unsubstituted 5-9-membered heteroaryl. In some embodiments, R⁷ is substituted or unsubstituted 5-9-membered heteroaryl fused with C₅-C₆ cycloalkyl. In some embodiments, R⁷ is alkoxy. In some embodiments, R⁷ is methoxy. In some embodiments, R⁷ is halogen.

In some embodiments, the substituted C₁-C₃ alkyl, substituted 5-6-membered heteroalkyl, substituted C₃-C₆ cycloalkyl, substituted C₃-C₆ cycloalkyl fused with aryl, substituted C₃-C₆ cycloalkyl fused with phenyl, substituted 3-6-membered heterocyclyl, substituted C₆-C₁₀ aryl, substituted phenyl, substituted C₆-C₁₀ aryl fused with C₅-C₆ cycloalkyl, substituted 5-9-membered heteroaryl, and substituted 5-9-membered heteroaryl fused with C₅-C₆ cycloalkyl of R⁷ are substituted with one or more substituent, one to five substituents, one to four substituents, one to three substituents, one or two substituents, or one substituent.

In some embodiments, the substituted C₁-C₃ alkyl, substituted 5-6-membered heteroalkyl, substituted C₃-C₆ cycloalkyl, substituted C₃-C₆ cycloalkyl fused with aryl, substituted C₃-C₆ cycloalkyl fused with phenyl, substituted 3-6-membered heterocyclyl, substituted C₆-C₁₀ aryl, substituted phenyl, substituted C₆-C₁₀ aryl fused with C₅-C₆ cycloalkyl, substituted 5-9-membered heteroaryl, and substituted 5-9-membered heteroaryl fused with C₅-C₆ cycloalkyl of R⁷, are substituted with substituents selected from the group consisting of R^{7a}, R^{7b}, R^{7c}, R^{7d}, and R^{7e}, wherein the R^{7a}, R^{7b}, R^{7c}, R^{7d}, and R^{7e} are each independently selected from the group consisting of hydrogen, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃-C₁₀ cycloalkyl, and substituted or unsubstituted 4-10 membered heterocyclyl, or wherein any two of R^{7a}, R^{7b}, R^{7c}, R^{7d}, and R^{7e} are connected to the phenyl at adjacent carbon atoms, and the two R^{7a}, R^{7b}, R^{7c}, R^{7d}, and R^{7e} are connected, together with the carbon atoms to which they are attached, to form a substituted or unsubstituted C₃-C₆ cycloalkyl, or a substituted or unsubstituted 5- or 6-membered heterocyclic ring comprising 1-4 heteroatoms selected from the group consisting of N, S, and O, fused to the phenyl,
wherein R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, C₃-C₁₀ cycloalkyl, heterocyclyl, heteroarylalkyl, and heteroaryl,
wherein R^{7a}, R^{7b}, R^{7c}, R^{7d}, and R^{7e} are each substituted with one or more substituents selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when R^{7a}, R^{7b}, R^{7c}, R^{7d}, or R^{7e} is a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃-C₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, C₃-C₁₀ cycloalkyl, heterocyclyl, and heteroaryl, and
wherein when one or more chiral center is present, each chiral center has (R) stereochemistry, (S) stereochemistry, or is a mixture thereof.

In some embodiments of Formulae (I), (I-A), (I-B), (II), (III), and (IV), R⁷ is unsubstituted C₆-C₁₀ aryl. In some embodiments, R⁷ is substituted C₆-C₁₀ aryl.

In some embodiments of Formulae (I), (I-A), (I-B), (II), (III), and (IV), R⁷ is unsubstituted phenyl. In some embodiments, R⁷ is substituted phenyl.

In some embodiments, the substituted C₆-C₁₀ aryl or substituted phenyl of R⁷ is substituted with substituents selected from the group consisting of R^{7a}, R^{7b}, R^{7c}, R^{7d}, and R^{7e}, wherein the R^{7a}, R^{7b}, R^{7c}, R^{7d}, and R^{7e} are each independently selected from the group consisting of hydrogen, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃-C₁₀ cycloalkyl, and substituted or unsubstituted 4-10 membered heterocyclyl, or wherein any two of R^{7a}, R^{7b}, R^{7c}, R^{7d}, and R^{7e} are connected to the phenyl at adjacent carbon atoms, and the two R^{7a}, R^{7b}, R^{7c}, R^{7d}, and R^{7e} are connected, together with the carbon atoms to which they are attached, to form a substituted or unsubstituted C₃-C₆ cycloalkyl, or a substituted or unsubstituted 5- or 6-membered heterocyclic ring comprising 1-4 heteroatoms selected from the group consisting of N, S, and O, fused to the phenyl,
wherein R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, C₃-C₁₀ cycloalkyl, heterocyclyl, heteroarylalkyl, and heteroaryl,
wherein R^{7a}, R^{7b}, R^{7c}, R^{7d}, and R^{7e} are each substituted with one or more substituents selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when R^{7a}, R^{7b}, R^{7c}, R^{7d}, or R^{7e} is a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃-C₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, C₃-C₁₀ cycloalkyl, heterocyclyl, and heteroaryl, and
wherein when one or more chiral center is present, each chiral center has (R) stereochemistry, (S) stereochemistry, or is a mixture thereof.

In some embodiments of Formulae (I), (I-A), (I-B), (II), (III), and (IV), R⁷ is unsubstituted 5-9-membered heteroaryl. In some embodiments of Formulae (I), (I-A), (I-B), (II), (III), and (IV), R⁷ is substituted 5-9-membered heteroaryl.

In some embodiments of Formulae (I), (I-A), (I-B), (II), (III), and (IV), the substituted 5-9-membered heteroaryl of R⁷ is substituted with one or more substituents independently selected from the group consisting of R^{7a}, R^{7b}, R^{7c}, R^{7d}, and R^{7e}, wherein the R^{7a}, R^{7b}, R^{7c}, R^{7d}, and R^{7e} are each independently selected from the group consisting of hydrogen, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃-C₁₀ cycloalkyl, and substituted or unsubstituted 4-10 membered heterocyclyl, or wherein any two of R^{7a}, R^{7b}, R^{7c}, R^{7d}, and R^{7e} are connected to the phenyl at adjacent carbon atoms, and the two R^{7a}, R^{7b}, R^{7c}, R^{7d}, and R^{7e} are connected, together with the carbon atoms to which they are attached, to form a substituted or unsubstituted C₃-C₆ cycloalkyl, or a substituted or unsubstituted 5- or 6-membered heterocyclic ring comprising 1-4 heteroatoms selected from the group consisting of N, S, and O, fused to the phenyl,
wherein R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, C₃-C₁₀ cycloalkyl, heterocyclyl, heteroarylalkyl, and heteroaryl,
wherein R^{7a}, R^{7b}, R^{7c}, R^{7d}, and R^{7e} are each substituted with one or more substituents selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when R^{7a}, R^{7b}, R^{7c}, R^{7d}, or R^{7e} is a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃-C₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, C₃-C₁₀ cycloalkyl, heterocyclyl, and heteroaryl, and
wherein when one or more chiral center is present, each chiral center has (R) stereochemistry, (S) stereochemistry, or is a mixture thereof.

In some embodiments of Formulae (I), (I-A), (I-B), (II), (III), and (IV), the substituted C₁-C₃ alkyl, substituted 5-6-membered heteroalkyl, substituted C₃-C₆ cycloalkyl, substituted C₃-C₆ cycloalkyl fused with aryl, substituted C₃-C₆ cycloalkyl fused with phenyl, substituted 3-6-membered heterocyclyl, substituted C₆-C₁₀ aryl, substituted phenyl, substituted C₆-C₁₀ aryl fused with C₅-C₆ cycloalkyl, substituted 5-9-membered heteroaryl, and substituted 5-9-membered heteroaryl fused with C₅-C₆ cycloalkyl of R⁷ are substituted with substituents selected from the group consisting of hydrogen, C₁-C₄ alkyl, halogen, phenyl, alkoxy, haloalkyl, hydroxyalkyl, C₃-C₆ cycloalkyl, cyano, alkylsulfonyl, and pyrazolyl.

In some embodiments of Formulae (I), (I-A), (I-B), (II), (III), and (IV), the substituted C₁-C₃ alkyl, substituted 5-6-membered heteroalkyl, substituted C₃-C₆ cycloalkyl, substituted C₃-C₆ cycloalkyl fused with aryl, substituted C₃-C₆ cycloalkyl fused with phenyl, substituted 3-6-membered heterocyclyl, substituted C₆-C₁₀ aryl, substituted phenyl, substituted C₆-C₁₀ aryl fused with C₅-C₆ cycloalkyl, substituted 5-9-membered heteroaryl, and substituted 5-9-membered heteroaryl fused with C₅-C₆ cycloalkyl of R⁷ are substituted with substituents selected from the group consisting of hydrogen, methyl, ethyl, isopropyl, fluoro, chloro, phenyl, methoxy, difluoromethyl, trifluoromethyl, hydroxymethyl, cyclopropyl, cyano, 2-hydroxy-2-propyl, methanesulfonyl, or pyrazolyl.

In some embodiments, the substituted C₆-C₁₀ aryl or substituted phenyl is substituted with one or more substituent, one to five substituents, one to four substituents, one to three substituents, one or two substituents, or one substituent.

In some embodiments of Formulae (I), (I-A), (I-B), (II), (III), and (IV), the substituted C₁-C₃ alkyl of R⁷ is substituted with one, two, or three fluoro groups.

In some embodiments of Formulae (I), (I-A), (I-B), (II), (III), and (IV), the substituted C₆-C₁₀ aryl of R⁷ is substituted with substituents selected from the group consisting of C₁-C₄ alkyl, halogen, phenyl, and combinations thereof.

In some embodiments of Formulae (I), (I-A), (I-B), (II), (III), and (IV), the C₅-cycloalkyl fused with substituted or unsubstituted phenyl of R⁷ is substituted with substituents selected from the group consisting of C₁-C₄ alkyl, halogen, phenyl, and combinations thereof.

In some embodiments of Formulae (I), (I-A), (I-B), (II), (III), and (IV), the substituted 5-9-membered heteroaryl of R⁷ is substituted with hydrogen.

In some embodiments of Formulae (I), (I-A), (I-B), (II), (III), and (IV), the substituted 5-9-membered heteroaryl of R⁷ is substituted with C₁-C₄ alkyl. In some embodiments of Formulae (I), (I-A), (I-B), (II), (III), and (IV), the substituted 5-9-membered heteroaryl of R⁷ is substituted with methyl, ethyl, isopropyl. In some embodiments of Formulae (I), (I-A), (I-B), (II), (III), and (IV), the substituted 5-9-membered heteroaryl of R⁷ is substituted with methyl.

In some embodiments of Formulae (I), (I-A), (I-B), (II), (III), and (IV), the substituted 5-9-membered heteroaryl of R⁷ is substituted with halogen. In some embodiments of Formulae (I), (I-A), (I-B), (II), (III), and (IV), the substituted 5-9-membered heteroaryl of R⁷ is substituted with fluoro or chloro.

In some embodiments of Formulae (I), (I-A), (I-B), (II), (III), and (IV), the substituted 5-9-membered heteroaryl of R⁷ is substituted with phenyl.

In some embodiments of Formulae (I), (I-A), (I-B), (II), (III), and (IV), the substituted 5-9-membered heteroaryl of R⁷ is substituted with alkoxy. In some embodiments of Formulae (I), (I-A), (I-B), (II), (III), and (IV), the substituted 5-9-membered heteroaryl of R⁷ is substituted with methoxy.

In some embodiments of Formulae (I), (I-A), (I-B), (II), (III), and (IV), the substituted 5-9-membered heteroaryl of R⁷ is substituted with haloalkyl. In some embodiments of Formulae (I), (I-A), (I-B), (II), (III), and (IV), the substituted 5-9-membered heteroaryl of R⁷ is substituted with fluoromethyl, difluoromethyl, or trifluoromethyl.

In some embodiments of Formulae (I), (I-A), (I-B), (II), (III), and (IV), the substituted 5-9-membered heteroaryl of R⁷ is substituted with hydroxyalkyl. In some embodiments of Formulae (I), (I-A), (I-B), (II), (III), and (IV), the substituted 5-9-membered heteroaryl of R⁷ is substituted with hydroxymethyl.

In some embodiments of Formulae (I), (I-A), (I-B), (II), (III), and (IV), the substituted 5-9-membered heteroaryl of R⁷ is substituted with C₃-C₆ cycloalkyl. In some embodiments of Formulae (I), (I-A), (I-B), (II), (III), and (IV), the substituted 5-9-membered heteroaryl of R⁷ is substituted with cyclopropyl.

In some embodiments of Formulae (I), (I-A), (I-B), (II), (III), and (IV), the substituted 5-9-membered heteroaryl of R⁷ is substituted with pyrazolyl.

In some embodiments of Formulae (I), (I-A), (I-B), (II), (III), and (IV), the substituted 5-9-membered heteroaryl of R⁷ is substituted with cyano.

In some embodiments of Formulae (I), (I-A), (I-B), (II), (III), and (IV), the substituted 5-9-membered heteroaryl of R⁷ is substituted with alkylsulfonyl. In some embodiments of Formulae (I), (I-A), (I-B), (II), (III), and (IV), the substituted 5-9-membered heteroaryl of R⁷ is substituted with methanesulfonyl.

In some embodiments of Formulae (I), (I-A), (I-B), (II), (III), and (IV), the substituted 5-9-membered heteroaryl of R⁷ is substituted with 2-hydroxy-2-propyl.

In some embodiments of Formula (III), R⁷ is a substituted or unsubstituted C₆-C₁₀ aryl, wherein the substituted C₆-C₁₀ aryl is substituted with one or more halogen, one or more C₁-C₃ alkyl, or a combination thereof.

In some embodiments of Formulae (I), (I-A), (I-B), (II), (III), and (IV), R⁷ is phenyl substituted with one or more of R^{7a}, R^{7b}, R^{7c}, R^{7d}, and R^{7e}, wherein the R^{7a}, R^{7b}, R^{7c}, R^{7d}, and R^{7e} are each independently selected from the group consisting of C₁-C₄ alkyl, halogen, phenyl, alkoxy, haloalkyl, hydroxyalkyl, or a combination thereof.

In some embodiments of Formulae (I), (I-A), (I-B), (II), (III), and (IV), the substituted phenyl of R⁷ is phenyl substituted with C₁-C₄ alkyl. In some embodiments of Formulae (I), (I-A), (I-B), (II), (III), and (IV), the substituted phenyl of R⁷ is phenyl substituted with methyl, ethyl, isopropyl.

In some embodiments of Formulae (I), (I-A), (I-B), (II), (III), and (IV), the substituted phenyl of R⁷ is phenyl substituted with halogen. In some embodiments of Formulae (I), (I-A), (I-B), (II), (III), and (IV), the substituted phenyl of R⁷ is phenyl substituted with fluoro or chloro.

In some embodiments of Formulae (I), (I-A), (I-B), (II), (III), and (IV), the substituted phenyl of R⁷ is phenyl substituted with phenyl.

In some embodiments of Formulae (I), (I-A), (I-B), (II), (III), and (IV), the substituted phenyl of R⁷ is phenyl substituted with alkoxy. In some embodiments of Formulae (I), (I-A), (I-B), (II), (III), and (IV), the substituted phenyl of R⁷ is phenyl substituted with methoxy.

In some embodiments of Formulae (I), (I-A), (I-B), (II), (III), and (IV), the substituted phenyl of R⁷ is phenyl substituted with haloalkyl. In some embodiments of Formulae (I), (I-A), (I-B), (II), (III), and (IV), the substituted phenyl of R⁷ is phenyl substituted with fluoromethyl, difluoromethyl, or trifluoromethyl.

In some embodiments of Formulae (I), (I-A), (I-B), (II), (III), and (IV), the substituted phenyl of R⁷ is phenyl substituted with hydroxyalkyl. In some embodiments of Formulae (I), (I-A), (I-B), (II), (III), and (IV), the substituted phenyl of R⁷ is phenyl substituted with hydroxymethyl.

In some embodiments of Formulae (I), (I-A), (I-B), (II), (III), and (IV), the substituted phenyl of R⁷ is phenyl substituted with C₃-C₆ cycloalkyl. In some embodiments of Formulae (I), (I-A), (I-B), (II), (III), and (IV), the substituted phenyl of R⁷ is phenyl substituted with cyclopropyl.

In some embodiments of Formulae (I), (I-A), (I-B), (II), (III), and (IV), the substituted phenyl of R⁷ is phenyl substituted with cyano.

In some embodiments of Formulae (I), (I-A), (I-B), (II), (III), and (IV), the substituted phenyl of R⁷ is phenyl substituted with alkylsulfonyl. In some embodiments of Formulae (I), (I-A), (I-B), (II), (III), and (IV), the substituted phenyl of R⁷ is phenyl substituted with methanesulfonyl.

In some embodiments of Formulae (I), (I-A), (I-B), (II), (III), and (IV), the substituted phenyl of R⁷ is phenyl substituted with pyrazolyl.

In some embodiments of Formulae (I), (I-A), (I-B), (II), (III), and (IV), the substituted phenyl of R⁷ is phenyl substituted with 2-hydroxy-2-propyl.

In some embodiments of Formulae (I), (I-A), (I-B), (II), (III), and (IV), R⁷ is a phenyl substituted at two adjacent carbon atoms of the phenyl to form, together with the carbon atoms to which they are attached, a phenyl fused with C₃-C₆ cycloalkyl. In some embodiments of Formulae (I), (I-A), (I-B), (II), (III), and (IV), R⁷ is a phenyl substituted at two adjacent carbon atoms of the phenyl to form, together with the carbon atoms to which they are attached, a phenyl fused with C₆-cycloalkyl.

In some embodiments of Formulae (I), (I-A), (I-B), (II), (III), and (IV), R⁷ has one of the structures:

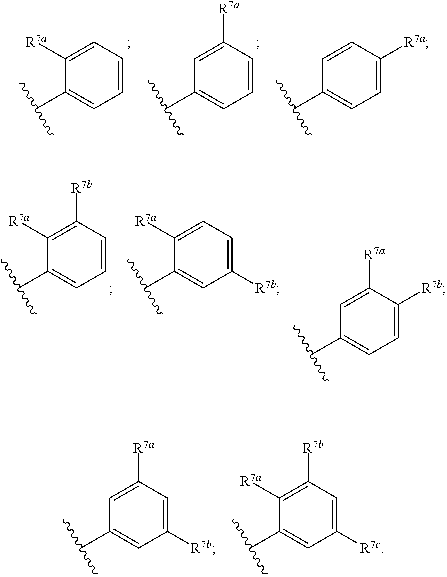

In some embodiments of Formulae (I), (I-A), (I-B), (II), (III), and (IV), R^{7a}, R^{7b}, and R^{7c} are independently selected from the group consisting of hydrogen, C₁-C₆ alkyl, halogen, phenyl, alkoxy, haloalkyl, hydroxyalkyl, C₃-C₆ cycloalkyl, cyano, alkylsulfonyl, and 5-membered heteroaryl.

In some embodiments of Formulae (I), (I-A), (I-B), (II), (III), and (IV), R^{7a}, R^{7b}, and R^{7c} are independently selected from the group consisting of hydrogen, methyl, ethyl, isopropyl, fluoro, chloro, phenyl, methoxy, difluoromethyl, trifluoromethyl, hydroxymethyl, cyclopropyl, cyano, 2-hydroxy-2-propyl, methanesulfonyl, and pyrazolyl.

In some embodiments of Formulae (I), (I-A), (I-B), (II), (III), and (IV), R^{7a}, R^{7b}, and R^{7c} are independently selected from the group consisting of hydrogen, C₁-C₆ alkyl, halo, and phenyl.

In some embodiments of Formulae (I), (I-A), (I-B), (II), (III), and (IV), R^{7a} and R^{7b}, when bound to adjacent carbon atoms of the phenyl, together with the carbon atoms to which they are attached, form a C₃-C₆ cycloalkyl fused with the phenyl. In some embodiments of Formulae (I), (I-A), (I-B), (II), (III), and (IV), R^{7a} and R^{7b}, when bound to adjacent carbon atoms of the phenyl, together with the carbon atoms to which they are attached, form a C₆ cycloalkyl fused with the phenyl.

In some embodiments of Formulae (I), (I-A), (I-B), (II), (III), and (IV), R⁷ is selected from the group consisting of:

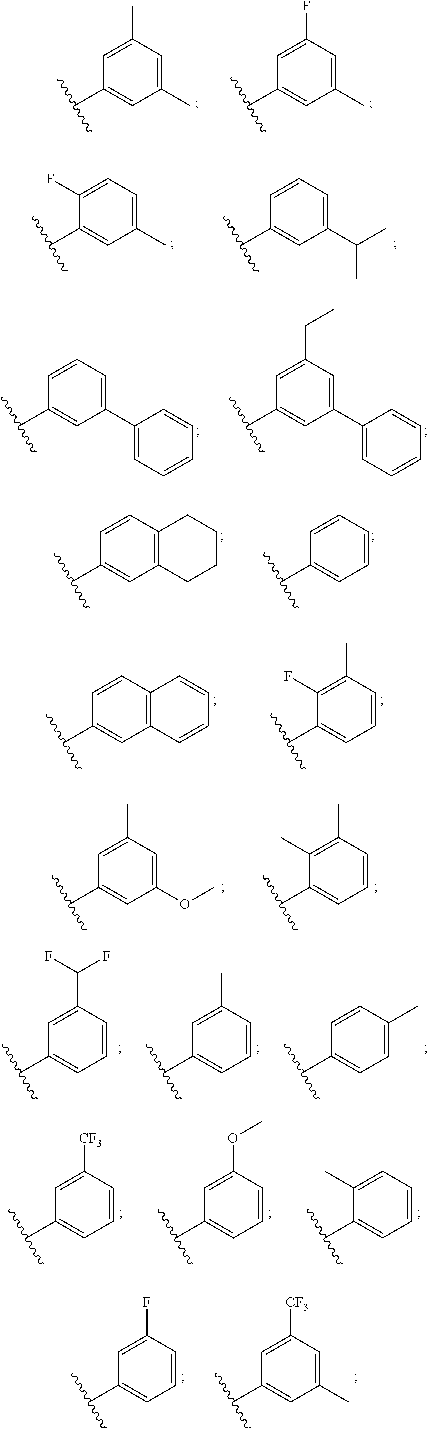

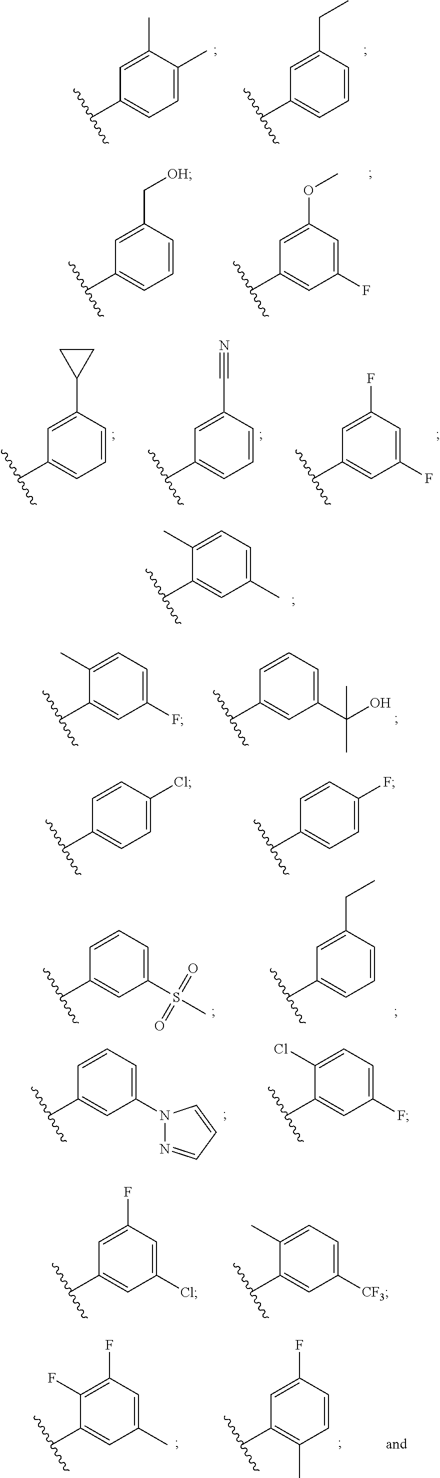

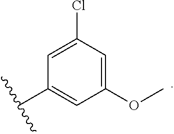 [SMILES: *c1cc(Cl)cc(OC)c1]

In some embodiments of Formula (II), R⁷ is selected from the group consisting of:

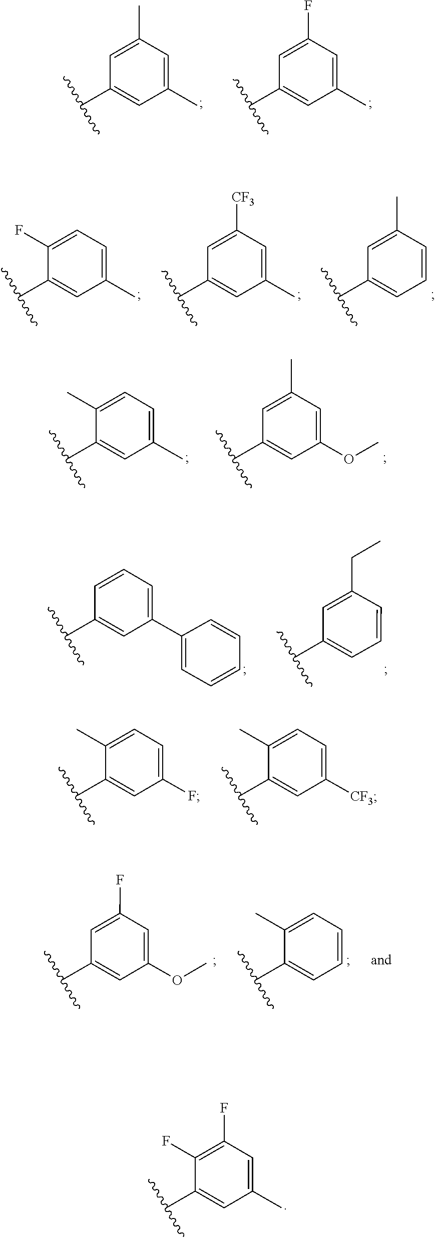

In some embodiments of Formula (III), R⁷ is selected from the group consisting of:

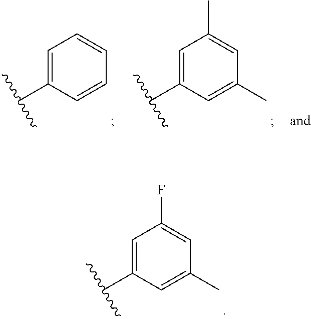

In some embodiments of Formula (IV), R⁷ is selected from the group consisting of:

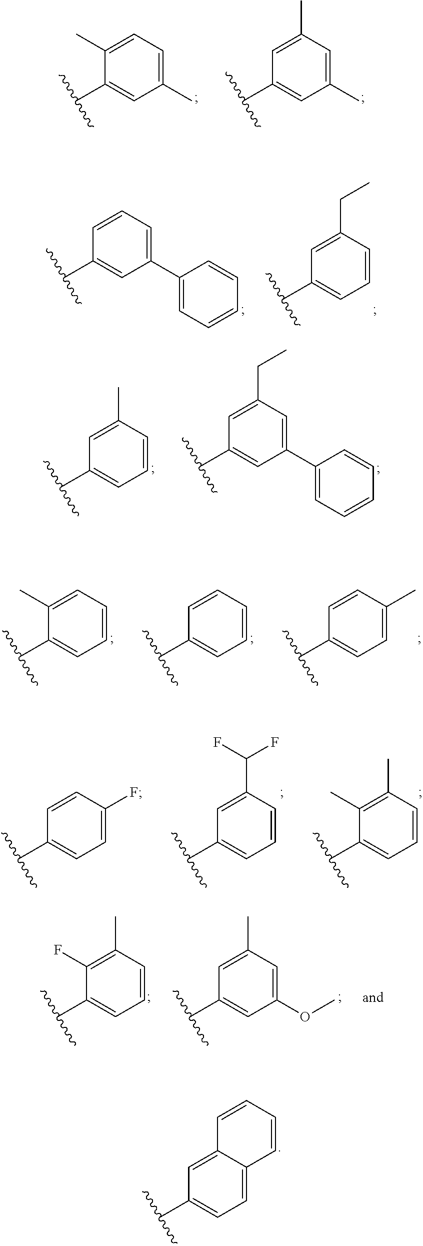

In some embodiments of Formulae (I), (I-A), (I-B), (II), (III), and (IV), R⁷ is selected from the group consisting of:

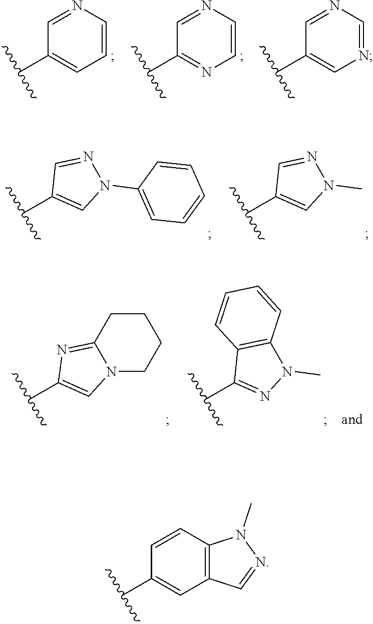

In some embodiments of Formulae (I), (I-A), (I-B), (II), (III), and (IV), R⁷ is selected from the group consisting of:

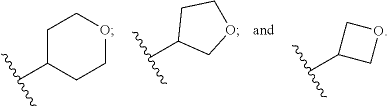

In some embodiments of Formulae (I), (I-A), (I-B), (II), (III), and (IV), R⁷ is selected from the group consisting of:

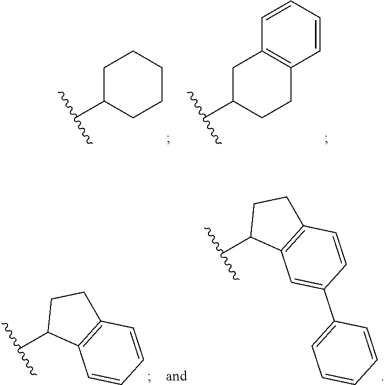

In some embodiments of Formulae (I), (I-A), (I-B), (II), (III), and (IV), R⁷ is selected from the group consisting of —OCH₃ and —CHF₂.

In some embodiments of Formulae (I), (I-A), (I-B), (II), (III), and (IV), R⁷ is hydrogen and n is 0.

In some embodiments of Formulae (I), (I-A), (I-B), (II), (III), and (IV), n is 0. In some embodiments, n is 1. In some embodiments, n is 2. In some embodiments, n is 3. In some embodiments, n is 4.

In some embodiments of Formulae (I), (I-A), (I-B), (II), (III), and (IV), when R⁷ comprises one or more chiral center, each chiral center of R⁷ has (R) stereochemistry, (S) stereochemistry, or is a mixture thereof.

In some embodiments of Formulae (I), (I-A), (I-B), (II), (III), and (IV), L is —(CR^{8a}R^{8b})ₙ—, wherein each —(CR^{8a}R^{8b})— is the same or is different.

In some embodiments of Formulae (I), (I-A), (I-B), (II), (III), and (IV), R^{8a} and R^{8b} are each independently selected from the group consisting of hydrogen, C₁-C₄ linear alkyl, C₃-C₄ branched alkyl, substituted or unsubstituted C₃-C₆ cycloalkyl, hydroxyalkyl, and alkoxy.

In some embodiments of Formulae (I), (I-A), (I-B), (II), (III), and (IV), R^{8a} and R^{8b} together, with the carbon to which they are attached, form a substituted or unsubstituted C₃-C₆ cycloalkyl. In some embodiments of Formulae (I), (I-A), (I-B), (II), (III), and (IV), R^{8a} and R^{8b} together, with the carbon to which they are attached, form a substituted or unsubstituted 3-6-membered heterocyclyl. In some embodiments of Formulae (I), (I-A), (I-B), (II), (III), and (IV), R^{8a} and R^{8b} together, with the carbon to which they are attached, form a substituted or unsubstituted 4-membered heterocyclyl.

In some embodiments of Formulae (I), (I-A), (I-B), (II), (III), and (IV), n is 3, one of R^{8a} or R^{8b} from a first carbon is a bridging —CH₂— to a third carbon, and one of R^{8a} or R^{8b} from the third carbon is absent, to form a 1,3-cyclobutylene.

In some embodiments of Formulae (I), (I-A), (I-B), (II), (III), and (IV), R^{8a} and R^{8b} are each independently hydrogen.

In some embodiments of Formulae (I), (I-A), (I-B), (II), (III), and (IV), R^{8a} and R^{8b} are each independently C₁-C₄ linear alkyl.

In some embodiments of Formulae (I), (I-A), (I-B), (II), (III), and (IV), R^{8a} and R^{8b} are each independently C₃-C₄ branched alkyl.

In some embodiments of Formulae (I), (I-A), (I-B), (II), (III), and (IV), R^{8a} and R^{8b} are each independently substituted or unsubstituted C₃-C₆ cycloalkyl.

In some embodiments of Formulae (I), (I-A), (I-B), (II), (III), and (IV), R^{8a} and R^{8b} are each independently hydroxyalkyl.

In some embodiments of Formulae (I), (I-A), (I-B), (II), (III), and (IV), R^{8a} and R^{8b} are each independently alkoxy.

In some embodiments of Formulae (I), (I-A), (I-B), (II), (III), and (IV), R^{8a} and R^{8b} are each independently

In some embodiments of Formulae (I), (I-A), (I-B), (II), (III), and (IV), R^{8a} and R^{8b} are each independently

In some embodiments of Formulae (I), (I-A), (I-B), (II), (III), and (IV), the substituted C₃-C₆ cycloalkyl of R^{8a} and R^{8b} are substituted with C₁-C₃ alkyl or C₁-C₃ alkoxy.

In some embodiments of Formulae (I), (I-A), (I-B), (II), (III), and (IV), the substituted 3-6-membered heterocyclyl of R^{8a} and R^{8b} are substituted with C₁-C₃ alkyl or C₁-C₃ alkoxy.

In some embodiments of Formulae (I), (I-A), (I-B), (II), (III), and (IV), L is selected from the group consisting of —CH₂—; —CH₂CH₂—; —CH₂CH₂CH₂—; —CH₂CH₂CH₂CH₂—; —CH(CH₃)—; —CH(CH₂CH₃)—; —CH(CH₂CH₂CH₃)—; —CH(CH(CH₃)₂)—; —CH(C(CH₃)₃)—; —CH₂CH(CH₃)—; —CH(CH₂OH)—; and —CH₂CH(OCH₃)—.

In some embodiments of Formulae (I), (I-A), (I-B), (II), (III), and (IV), L is selected from the group consisting of

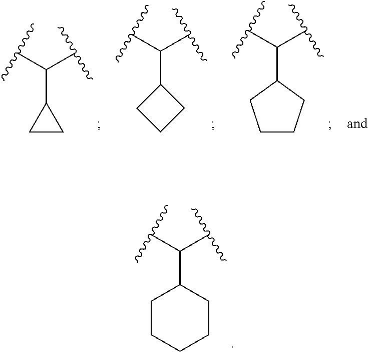 [SMILES: *C(*)C1CC1 ; *C(*)C1CCC1 ; *C(*)C1CCCC1 ; *C(*)C1CCCCC1]

In some embodiments of Formulae (I), (I-A), (I-B), (II), (III), and (IV),

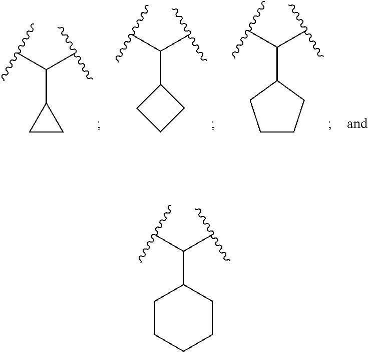 [SMILES: *C(*)C1CC1 ; *C(*)C1CCC1 ; *C(*)C1CCCC1 ; *C(*)C1CCCCC1]

and are substituted or unsubstituted, and wherein the substituted

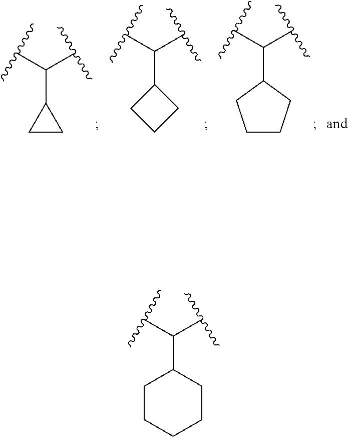 [SMILES: *C(*)C1CC1 ; *C(*)C1CCC1 ; *C(*)C1CCCC1 ; *C(*)C1CCCCC1]

and are substituted with hydroxy, alkoxy, amino, halo, cyano, alkyl, alkylsulfonyl, or a combination thereof.

In some embodiments of Formulae (I), (I-A), (I-B), (II), (III), and (IV), L is selected from the group consisting of

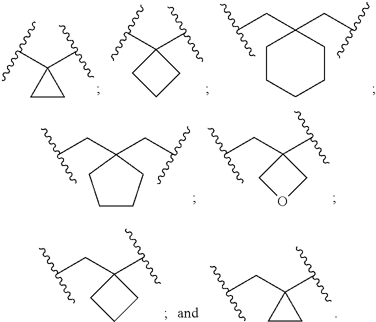

In some embodiments of Formulae (I), (I-A), (I-B), (II), (III), and (IV), the

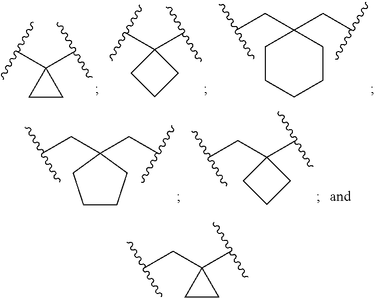

are substituted or unsubstituted. In some embodiments of Formulae (I), (I-A), (I-B), (II), (III), and (IV), the substituted

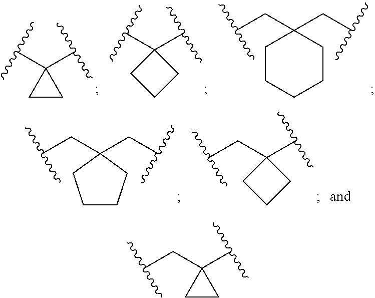

and are substituted with hydroxy, alkoxy, amino, halo, cyano, alkyl, alkylsulfonyl, or a combination thereof.

In some embodiments of Formulae (I), (I-A), (I-B), (II), (III), and (IV), L is

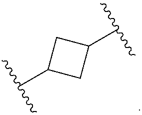 [SMILES: *C1CC(*)C1]

In some embodiments of Formulae (I), (I-A), (I-B), (II), (III), and (IV), L is selected from the group consisting of —CH₂—; —CH₂CH₂—; —CH₂CH₂CH₂—; —CH₂CH₂CH₂CH₂—; —CH(CH₃)—; —CH(CH₂CH₃)—; —CH(CH(CH₃)₂)—; —CH(C(CH₃)₃)—; —CH(CH₂OH)—; —CH₂CH(OCH₃)—;

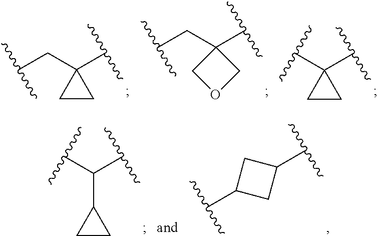 [SMILES: *CC1(*)CC1 ; *CC1(*)COC1 ; *C1(*)CC1 ; *C(*)C1CC1 ; *C1CC(*)C1]

and wherein when a chiral center is present, the L group comprises (R) stereochemistry, (S) stereochemistry, or a mixture thereof.

In some embodiments of Formulae (I), (I-A), (I-B), (II), (III), and (IV), L is selected from the group consisting of —CH₂—; —CH₂CH₂—; —CH₂CH₂CH₂—; —CH₂CH₂CH₂CH₂—; —CH(CH₃)—; —CH(CH₂CH₃)—; —CH(CH₂CH₂CH₃)—; —CH(CH(CH₃)₂)—; —CH(C(CH₃)₃)—; —CH₂CH(CH₃)—; —CH(CH₂OH)—; —CH₂CH(OCH₃)—;

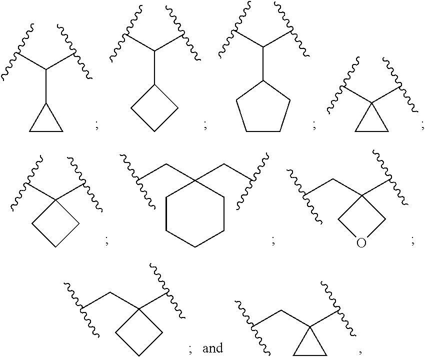

and wherein when a chiral center is present, the L group comprises (R) stereochemistry, (S) stereochemistry, or a mixture thereof.

In some embodiments, L is —CH₂—.

In some embodiments, L is —CH(CH₃)—.

In some embodiments, L is —CH₂CH₂—.

In some embodiments, L is —CH₂CH₂CH₂—.

In some embodiments, L is —CH(CH₂CH₃)—.

In some embodiments, when one or more chiral center is present in L, each chiral center has (R) stereochemistry, (S) stereochemistry, or is a mixture thereof.

In some embodiments of Formulae (I), (I-A), (I-B), (II), (III), and (IV), R² and L, together with the nitrogen to which they are attached, form a substituted or unsubstituted 3-6-membered heterocyclyl.

In some embodiments of Formulae (I), (I-A), (I-B), (II), (III), and (IV), R² and L, together with the nitrogen to which they are attached, form a 4-membered heterocyclyl.

In some embodiments of Formulae (I), (I-A), (I-B), (II), (III), and (IV), R² and L, together with the nitrogen to which they are attached, form a compound having the Formula (I-C):

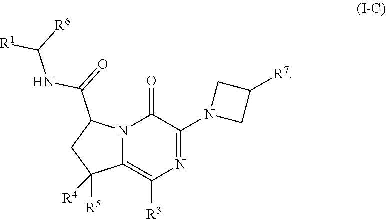

In some aspects, the present disclosure provides a compound having Formula (V):

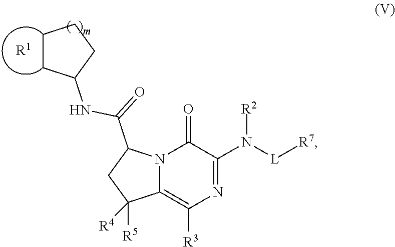

or a stereoisomer, tautomer, or pharmaceutically acceptable salt thereof, wherein:
R¹ is a substituted or unsubstituted aryl, or a substituted or unsubstituted 5-10-membered heteroaryl;
R² is hydrogen, or R² and L, together with the nitrogen to which they are attached, form a substituted or unsubstituted 3-6-membered heterocyclyl;
R³ is selected from the group consisting of hydrogen, substituted or unsubstituted C₁-C₃ alkyl, substituted or unsubstituted C₃-C₆ cycloalkyl, substituted or unsubstituted phenyl, substituted or unsubstituted 5-6-membered heteroaryl, halogen, cyano, and CD₃, wherein the substituted C₁-C₃ alkyl, C₃-C₆ cycloalkyl, phenyl, and 5-6-membered heteroaryl of R³ are substituted with methyl, fluoroalkyl, difluoroalkyl, or trifluoroalkyl;
R⁴ is selected from the group consisting of hydrogen, C₁-C₆ alkyl, and benzyl, and R⁵ is hydrogen or methyl; or R⁴ and R⁵, together with the carbon to which they are attached, form a C₃-C₆ cycloalkyl or a 6-membered cycloalkenyl;
R⁷ is selected from the group consisting of hydrogen, substituted or unsubstituted C₁-C₃ alkyl, 6-membered heteroalkyl, substituted or unsubstituted C₃-C₆ cycloalkyl, substituted or unsubstituted C₃-C₆ cycloalkyl fused with C₆-C₁₀ aryl, substituted or unsubstituted 3-6-membered heterocyclyl, substituted or unsubstituted phenyl, substituted or unsubstituted phenyl fused with C₅-C₆ cycloalkyl, substituted or unsubstituted 5-9-membered heteroaryl, substituted or unsubstituted 5-9-membered heteroaryl fused with C₅-C₆ cycloalkyl, methoxy, and halogen, and wherein when R⁷ is hydrogen, n is 0;
L is —(CR^{8a}R^{8b})ₙ—, wherein each —(CR^{8a}R^{8b})— is the same or is different, wherein R^{8a} and R^{8b} are each independently selected from the group consisting of hydrogen, C₁-C₄ linear or branched alkyl, hydroxyalkyl, and alkoxy, or R^{8a} and R^{8b} together with the carbon to which they are attached form a C₃-C₆ cycloalkyl or a 4-membered heterocyclyl; and
n is 0, 1, 2, 3, or 4, and
wherein when one or more chiral center is present, each chiral center has (R) stereochemistry, (S) stereochemistry, or is a mixture thereof.

In some embodiments of Formula (V), m is 1.

In some embodiments of Formula (V), R² is hydrogen.

In some embodiments of Formula (V), R³ is hydrogen.

In some embodiments of Formula (V), R⁴ is hydrogen

In some embodiments of Formula (V), R⁵ is hydrogen.

In some embodiments the compound has a structure of Formula (V-A):

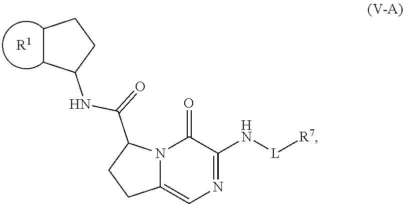

or a stereoisomer, tautomer, or pharmaceutically acceptable salt thereof.

In some embodiments of Formula (V) and Formula (V-A), R¹ has a structure selected from the group consisting of:

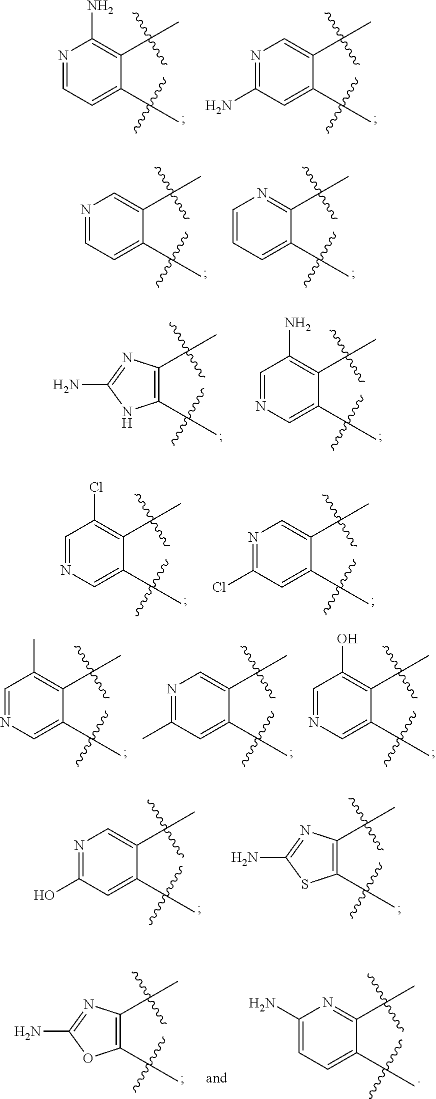

In some embodiments of Formula (V) and Formula (V-A), R¹ is

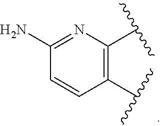 [SMILES: *c1ccc(N)nc1*]

In some embodiments, the compound has a structure of Formula (V-B):

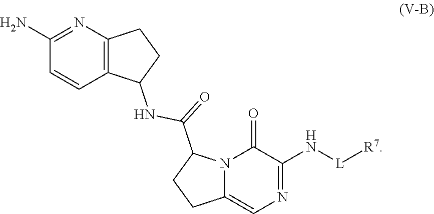

In some embodiments of Formula (V), (V-A), and (V-B), L is —CH₂—, —CH₂CH₂—, —CH(CH₃)—, or —CH₂CH₂CH₂—.

In some embodiments of Formula (V), (V-A), and (V-B), L is —CH₂—.

In some embodiments of Formula (V), (V-A), and (V-B), R⁷ is substituted or unsubstituted phenyl. In some embodiments of Formula (V), (V-A), and (V-B), R⁷ is phenyl substituted with one or more of R^{7a}, R^{7b}, R^{7c}, R^{7d}, and R^{7e}, wherein the R^{7a}, R^{7b}, R^{7c}, R^{7d}, and R^{7e} are each independently selected from the group consisting of C₁-C₄ alkyl, halogen, phenyl, alkoxy, haloalkyl, hydroxyalkyl, or a combination thereof. In some embodiments of Formula (V), (V-A), and (V-B), R⁷ is phenyl substituted at two adjacent carbon atoms form, together with the carbon atoms to which they are attached, a C₃-C₆ cycloalkyl fused with the phenyl.

In some embodiments of Formula (V), (V-A), and (V-B), R⁷ is phenyl independently substituted with one or more C₁-C₄ alkyl, halogen, phenyl, or a combination thereof.

In some embodiments of Formulae (I), (I-A), (I-B), (I-C), (II), (III), (IV), (V), (V-A), (V-B), one or more hydrogen atoms are replaced with one or more deuterium atoms.

The compounds of Formulae (I), (I-A), (I-B), (I-C), (II), (III), (IV), (V), (V-A), (V-B), and embodiments thereof, are useful in the methods and uses of the disclosure, optionally in the form of a salt, such as a pharmaceutically acceptable salt.

In some embodiments of Formulae (I), (I-A), (I-B), (I-C), (II), (III), (IV), (V), (V-A), (V-B), disclosed herein is a pharmaceutically acceptable salt of the indicated chemical compound (e.g., a hydrogen halide, such as a hydrogen chloride). Examples of pharmaceutically acceptable salts are set forth in, e.g., Burge, S. M. et al., J. Pharm. Sci 1977, 66, 1-19. Pharmaceutically acceptable salts include chlorides, bromides, iodides, formates, acetates, propionates, oxalates, malonates, succinates, fumarates, maleates, tartrates, citrates, benzoates, phthalates, sulfonates, arylsulfonates, alkylsulfonates, salts of fatty acids, and the like. Salts can be prepared by a variety of methods known to the skilled artisan, including a precipitation with, or an in-solution exposure to, an acid (e.g., treatment with gaseous HCl or an HCl solution), or a base.

In some embodiments of Formulae (I), (I-A), (I-B), (I-C), (II), (III), (IV), (V), (V-A), (V-B), the salt of the pharmaceutically acceptable salt is selected from the group consisting of trifluoroacetic acid, hydrogen chloride, acetic acid, hydrogen bromide, sulfuric acid, phosphoric acid, maleic acid, fumaric acid, lactic acid, tartaric acid, citric acid, and gluconic acid.

In some embodiments of Formulae (I-A), (I-B), (I-C), (II), (III), (IV), (V), (V-A), (V-B), the salt of the pharmaceutically acceptable salt is a trifluoroacetic acid salt. In some embodiments of Formulae (I-A), (I-B), (I-C), (II), (III), (IV), (V), (V-A), (V-B), the salt of the pharmaceutically acceptable salt is a hydrogen chloride salt. In some embodiments of Formulae (I-A), (I-B), (I-C), (II), (III), (IV), (V), (V-A), (V-B), the salt of the pharmaceutically acceptable salt is an acetic acid salt.

In some aspects, the compound of the disclosure is a compound of Table 1 below.

In some embodiments, the compound of Formulae (I-A), (I-B), (I-C), (II), (III), (IV), (V), (V-A), (V-B), is selected from one or more compounds of Formulae (I-A), (I-B), (I-C), (II), (III), (IV), (V), (V-A), (V-B), and embodiments thereof, as set forth in the Examples, including the compounds listed in Table 1.

In some embodiments, the compounds of Formulae (I-A), (I-B), (I-C), (II), (III), (IV), (V), (V-A), (V-B), and embodiments thereof, are provided in the form of a pharmaceutical composition comprising the compound, a stereoisomer, tautomer, or a salt thereof, such as a pharmaceutically acceptable salt, and at least one pharmaceutically acceptable carrier or excipient.

In some embodiments, the compounds of Formulae (I-A), (I-B), (I-C), (II), (III), (IV), (V), (V-A), (V-B), and embodiments thereof, are set forth as a stereoisomerically pure enantiomer or diastereomer (e.g., an optically active compound with one or more stereocenter(s)). Unless specifically indicated otherwise, any compound with one or more stereocenters is intended to include and to describe each of the pure (+) and (−) enantiomers, any other diastereomers, mixtures that are enriched in an enantiomer or diastereomer (e.g., 5%, 10%, 20%, 30%, 40%, 50%, 60%, 70%, 75%, 80%, 85%, 90%, 95%, 98%, or 99% enantiomeric or diastereomeric excess), and a racemic mixture of enantiomers or equal mixture of diastereomers.

In some embodiments, the compound is a prodrug. A prodrug is a compound that is converted to a biologically active form under physiological conditions, often by hydrolysis, oxidation, or reduction (e.g., ester to acid form; carbamate to amino or hydroxy group; hydroxyamidine to amidine) Exemplary prodrugs are set forth in, e.g., Tilley, J. W., “Prodrugs of Benzamide,” Prodrugs 2007, 191-222; Peterlin-Masic et al. Curr. Pharma. Design 2006, 12, 73-91. Prodrugs for the amidine group include amidoximes, O-alkylamidoximes, acylamidines, carbamates, 1,2,4-oxadiazolin-4-ones, and the like.

The compounds of Formulae (I-A), (I-B), (I-C), (II), (III), (IV), (V), (V-A), (V-B), and embodiments thereof, are useful as inhibitors of MASP-2 and for therapeutic use. The compounds of Formulae (I-A), (I-B), (I-C), (II), (III), (IV), (V), (V-A), (V-B), and embodiments thereof, are useful in methods of treating a disease or disorder treatable by inhibiting MASP-2. The compounds of the disclosure are useful in methods for inhibiting MASP-2. The compounds of the disclosure are useful in methods for inhibiting MASP-2 complement activation in a subject. The compounds of the disclosure are useful in methods for inhibiting MASP-2 or MASP-2 complement activation in a subject by administering to the subject a therapeutically effective amount of the compound effective to inhibit MASP-2 or MASP-2 complement activation. The compounds of Formulae (I-A), (I-B), (I-C), (II), (III), (IV), (V), (V-A), (V-B), and embodiments thereof, are useful in the manufacture of medicaments for treating a disease or disorder treatable by inhibiting MASP-2. The present disclosure also provides methods of treating a disease or disorder treatable by inhibiting MASP-2, comprising administering to a subject in need thereof a therapeutically effective amount of a compound of Formulae (I-A), (I-B), (I-C), (II), (III), (IV), (V), (V-A), (V-B), and embodiments thereof.

In some embodiments, the compound of Formulae (I-A), (I-B), (I-C), (II), (III), (IV), (V), (V-A), (V-B), and embodiments thereof, is used after a subject has been diagnosed as being in need of treatment for a lectin complement-associated disease or disorder.

In some embodiments, the compounds of Formulae (I-A), (I-B), (I-C), (II), (III), (IV), (V), (V-A), (V-B), and embodiments thereof, are selected as compounds which exhibit selectivity for MASP-2 over thrombin, the method comprising administering the compound as described herein. In some embodiments, the selectivity ratio of MASP-2:thrombin is at least 1.1:1, 1.25:1, 1.5:1, 1.75:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, or 30:1.

### III. Methods of Inhibiting MASP-2

In some aspects, the present disclosure provides a method for inhibiting MASP-2 in a subject, comprising administering to the subject a compound of Formula (I):

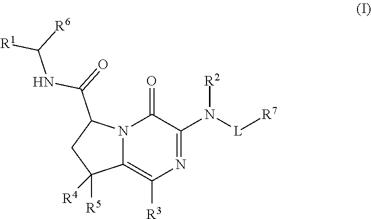

or a stereoisomer, tautomer, or pharmaceutically acceptable salt thereof, wherein:
R¹ is selected from a substituted or unsubstituted C₆-C₁₀ aryl and a substituted or unsubstituted 5-10-membered heteroaryl consisting of a 5-10-membered monocyclic or bicyclic ring system comprising at least one aromatic ring and one to six heteroatoms selected from the group consisting of nitrogen, oxygen, and sulfur, or
R¹ and R⁶, together with the carbon to which they are attached, form a C₃-C₆ cycloalkyl fused to a 5-10-membered heteroaryl or C₆-C₁₀ aryl, wherein the 5-10-membered heteroaryl or C₆-C₁₀ aryl, of the C₃-C₆ cycloalkyl fused to the 5-10-membered heteroaryl or C₆-C₁₀ aryl, is substituted or unsubstituted, and wherein the C₃-C₆ cycloalkyl, of the C₃-C₆ cycloalkyl fused to a 5-10-membered heteroaryl or C₆-C₁₀ aryl, is substituted or unsubstituted;
R² is hydrogen, or R² and L, together with the nitrogen to which they are attached, form a substituted or unsubstituted 3-6-membered heterocyclyl;
L is —(CR^{8a}R^{8b})ₙ—, wherein each —(CR^{8a}R^{8b})— is the same or is different;
R³ is selected from the group consisting of hydrogen, substituted or unsubstituted C₁-C₃ alkyl, substituted or unsubstituted C₃-C₆ cycloalkyl, substituted or unsubstituted 5-6-membered saturated or unsaturated heterocyclyl, substituted or unsubstituted phenyl, substituted or unsubstituted 5-6-membered heteroaryl, halogen, and cyano;
R⁴ and R⁵ are each independently selected from the group consisting of hydrogen, substituted or unsubstituted C₁-C₆ alkyl, and substituted or unsubstituted benzyl, or R⁴ and R⁵, together with the carbon to which they are attached, form a substituted or unsubstituted C₃-C₆ cycloalkyl or a substituted or unsubstituted 5-6-membered cycloalkenyl;
R⁶ is selected from the group consisting of hydrogen, C₁-C₃ alkyl, C₃-cycloalkyl, and cyano;
R⁷ is selected from the group consisting of hydrogen, substituted or unsubstituted C₁-C₃ alkyl, substituted or unsubstituted 5-6-membered heteroalkyl, substituted or unsubstituted C₃-C₆ cycloalkyl, substituted or unsubstituted C₃-C₆ cycloalkyl fused with aryl, substituted or unsubstituted 3-6-membered heterocyclyl, substituted or unsubstituted C₆-C₁₀ aryl, substituted or unsubstituted C₆-C₁₀ aryl fused with C₅-C₆ cycloalkyl, substituted or unsubstituted 5-9-membered heteroaryl, substituted or unsubstituted 5-9-membered heteroaryl fused with C₅-C₆ cycloalkyl, alkoxy, and halogen, and wherein when R⁷ is hydrogen, n is 0;
R^{8a} and R^{8b} are each independently selected from the group consisting of hydrogen, C₁-C₄ linear alkyl, C₃-C₄ branched alkyl, substituted or unsubstituted C₃-C₆ cycloalkyl, hydroxyalkyl, and alkoxy; or R^{8a} and R^{8b} together, with the carbon to which they are attached, form a substituted or unsubstituted C₃-C₆ cycloalkyl or a substituted or unsubstituted 3-6-membered heterocyclyl; or n is 3, one of R^{8a} or R^{8b} from a first carbon is a bridging —CH₂— to a third carbon, and one of R^{8a} or R^{8b} from the third carbon is absent, to form a 1,3-cyclobutylene; and
n is 0, 1, 2, 3, or 4, and
wherein when one or more chiral center is present, each chiral center has (R) stereochemistry, (S) stereochemistry, or is a mixture thereof, and
wherein all the definitions and embodiments of a compound of Formula (I) are as described herein.

In some embodiments, the present disclosure provides a method for inhibiting MASP-2 in a subject, comprising administering to the subject a compound of Formulae (I), (I-A), (I-B), (I-C), (II), (III), (IV), (V), (V-A), (V-B), as described herein.

In some embodiments, the present disclosure provides a method for inhibiting MASP-2 in a subject, comprising administering to the subject a compound of Formulae (I), (I-A), (I-B), (I-C), (II), (III), (IV), (V), (V-A), (V-B), as described herein, and wherein one or both of R³ and R⁶ is not H; one of R⁴ or R⁵ is substituted or unsubstituted benzyl; or R⁴ and R⁵, together with the carbon to which they are attached, form a substituted or unsubstituted 3-6-membered cycloalkyl, or a substituted or unsubstituted 5-6-membered cycloalkenyl.

In some embodiments, the present disclosure provides a method for inhibiting MASP-2 in a subject, comprising administering to the subject a compound of Formulae (I), (I-A), (I-B), (I-C), (II), (III), (IV), (V), (V-A), (V-B), as described herein, and wherein at least one of R³, R⁴, and R⁵ is not hydrogen, and R³ is not Cl when R¹ is

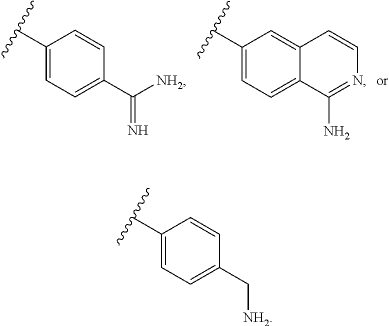 [SMILES: *c1ccc(C(=N)N)cc1 ; *c1ccc2c(N)nccc2c1 ; *c1ccc(CN)cc1]

### IV. Methods of Treatment

In some aspects, the present disclosure provides a method of treating a subject suffering from, or at risk for developing, a MASP-2-associated disease or disorder, such as a MASP-2-dependent complement-associated disease or disorder, comprising administering an inhibitor of MASP-2.

In some embodiments, provided herein is a method for inhibiting MASP-2 in a subject, comprising administering to the subject a compound of any one of Formulae (I), (I-A), (I-B), (I-C), (II), (III), (IV), (V), (V-A), (V-B), and embodiments thereof, as disclosed herein, or a stereoisomer, tautomer, or pharmaceutically acceptable salt thereof, in an amount effective to inhibit MASP-2.

In some embodiments, provided herein is a method for treating a disease or disorder treatable by inhibiting MASP-2, comprising administering to the subject a compound of any one of Formulae (I), (I-A), (I-B), (I-C), (II), (III), (IV), (V), (V-A), (V-B), and embodiments thereof, as disclosed herein, or a stereoisomer, tautomer, or pharmaceutically acceptable salt thereof.

In some embodiments, provided herein is a use of a compound of Formulae (I), (I-A), (I-B), (I-C), (II), (III), (IV), (V), (V-A), (V-B), and embodiments thereof, as disclosed herein, or a stereoisomer, tautomer, or pharmaceutically acceptable salt thereof, in the manufacture of a medicament for the treatment of a disease or disorder treatable by inhibiting MASP-2.

In some embodiments, provided herein is a method for treating a disease or disorder treatable by inhibiting MASP-2, comprising administering to a subject in need thereof a therapeutically effective amount of a compound of any one of Formulae (I), (I-A), (I-B), (I-C), (II), (III), (IV), (V), (V-A), (V-B), or a stereoisomer, tautomer, or pharmaceutically acceptable salt thereof.

In some embodiments, provided herein is a method wherein the compound of Formulae (I), (I-A), (I-B), (I-C), (II), (III), (IV), (V), (V-A), (V-B), or a stereoisomer, tautomer, or pharmaceutically acceptable salt thereof, is administered in an amount sufficient to inhibit MASP-2 dependent complement activation in the subject.

In some embodiments, the subject has been diagnosed as being in need of treatment for a lectin complement-associated disease or disorder.

The compound can be a compound of Formulae (I), (I-A), (I-B), (I-C), (II), (III), (IV), (V), (V-A), (V-B), and embodiments thereof, as disclosed herein. For example, in some embodiments, the compound is a compound of Formulae (I), (I-A), (I-B), (I-C), (II), (III), (IV), (V), (V-A), (V-B), and embodiments thereof, as described in any of the embodiments herein.

As described in U.S. Pat. Nos. 7,919,094; 8,840,893; 8,652,477; 8,951,522, 9,011,860, 9,475,885, 9,644,035, 9,644,035, 10,736,960, 10,059,776, 10,870,708; U.S. Patent Application Publication Nos. U.S. 2013/0344073, U.S. 2015/0166675, U.S. 2017/0137537, U.S. 2017/0166660, U.S. 2017/0253667, U.S. 2018/0105604, and U.S. 2020/0140570; and PCT Publication Nos. WO 2018/045054 and WO 2019/036460 (each of which Omeros Corporation was the Applicant, the Applicant of the instant application; each of which, except U.S. Pat. No. 10,870,708 Omeros Corporation was the Assignee, the Assignee of the instant application; and each of which is hereby incorporated by reference in its entirety), MASP-2-dependent complement activation has been implicated as contributing to the pathogenesis of numerous acute and chronic disease states. For example, as described in U.S. Pat. No. 8,951,522, the primary function of the complement system, a part of the innate immune system, is to protect the host against infectious agents.

However, inappropriate or over-activation of the complement system can lead to serious disease, such as thrombotic microangiopathies (TMAs, including aHUS, TTP and HUS) in which endothelial damage, as well as fibrin and platelet-rich thrombi in the microvasculature, leads to organ damage.

The lectin pathway plays a dominant role in activating complement under conditions of endothelial cell stress or injury, and prevents the activation of MASP-2. The lectin pathway halts the sequence of enzymatic reactions that leads to the formation of the membrane attack complex, platelet activation, and leukocyte recruitment. As described in U.S. Pat. No. 8,652,477, in addition to initiation of the lectin pathway, MASP-2 can also activate the coagulation system, wherein prothrombin is cleaved to thrombin.

Accordingly, in some embodiments, the method comprises administering to a patient suffering from, or at risk for developing, a MASP-2-dependent complement-associated disease or disorder, an amount of a compound of Formulae (I), (I-A), (I-B), (I-C), (II), (III), (IV), (V), (V-A), (V-B), and embodiments thereof, in an amount sufficient to inhibit MASP-2 dependent complement activation in a mammalian subject, to thereby treat the disease or disorder. In some embodiments, the method can further comprise, prior to administering a compound of Formulae (I), (I-A), (I-B), (I-C), (II), (III), (IV), (V), (V-A), (V-B), and embodiments thereof to a subject, determining that the patient is afflicted with the lectin complement-associated disease or disorder.

In some embodiments, the MASP-2-dependent complement-associated disease or disorder is selected from the group consisting of a thrombotic microangiopathy (TMA), a renal condition, an inflammatory reaction resulting from tissue or organ transplantation, an ischemia reperfusion injury, a complication associated with diabetes, a cardiovascular disease or disorder, an inflammatory gastrointestinal disorder, a pulmonary disorder, an ophthalmic disease or disorder, disseminated intravascular coagulation, graft-versus-host disease, veno-occlusive disease, diffuse alveolar hemorrhage, idiopathic pneumonia syndrome, capillary leak syndrome, engraftment syndrome, fluid overload, and a combination thereof.

In some embodiments, the MASP-2-dependent complement-associated disease or disorder is selected from the group consisting of a thrombotic microangiopathy (TMA) thrombotic thrombocytopenic purpura (TTP), refractory TTP, Upshaw-Schulman Syndrome (USS), hemolytic uremic syndrome (HUS), atypical hemolytic syndrome (aHUS), non-Factor H-dependent atypical hemolytic syndrome, aHUS secondary to an infection, plasma therapy-resistant aHUS, a TMA secondary to cancer, a TMA secondary to chemotherapy, a TMA secondary to transplantation, a TMA associated with hematopoietic stem cell transplant, and a combination thereof.

In some embodiments, the MASP-2-dependent complement-associated disease or disorder is graft-versus-host disease. In some embodiments, the method comprises administering to a patient suffering from or at risk for developing graft-versus-host disease (GVHD), including acute GVHD, chronic GVHD or steroid-resistant GVHD an amount of a compound of the disclosure in an amount sufficient to inhibit MASP-2 dependent complement activation in the mammalian subject to thereby treat the disease or disorder. In some embodiments, the subject suffering from, or at risk for developing, GVHD has previously undergone, is undergoing, or will undergo a hematopoietic stem cell transplant.

In some embodiments, the MASP-2-dependent complement-associated disease or disorder is diffuse alveolar hemorrhage (DAH). In some embodiments, the method comprises administering to a patient suffering from, or at risk for developing diffuse alveolar hemorrhage (DAH) an amount of a compound of the disclosure in an amount sufficient to inhibit MASP-2 dependent complement activation in the mammalian subject to thereby treat the disease or disorder. In some embodiments, the subject suffering from, or at risk for developing DAH has previously undergone, is undergoing, or will undergo a hematopoietic stem cell transplant.

In some embodiments, the MASP-2-dependent complement-associated disease or disorder is veno-occlusive disease (VOD). In some embodiments, the method comprises administering to a patient suffering from, or at risk for developing VOD an amount of a compound of the disclosure in an amount sufficient to inhibit MASP-2 dependent complement activation in the mammalian subject to thereby treat the disease or disorder. In some embodiments, the subject suffering from, or at risk for developing, VOD has previously undergone, is undergoing, or will undergo a hematopoietic stem cell transplant.

In some embodiments, the method comprises administering to a patient suffering from, or at risk for developing idiopathic pneumonia syndrome (IPS) an amount of a compound of the disclosure in an amount sufficient to inhibit MASP-2 dependent complement activation in the mammalian subject to thereby treat the disease or disorder. In some embodiments, the subject suffering from, or at risk for developing, IPS has previously undergone, is undergoing, or will undergo a hematopoietic stem cell transplant.

In some embodiments, the method comprises administering to a patient suffering from, or at risk for developing capillary leak syndrome (CLS) an amount of a compound of the disclosure in an amount sufficient to inhibit MASP-2 dependent complement activation in the mammalian subject to thereby treat the disease or disorder. In some embodiments, the subject suffering from, or at risk for developing CLS has previously undergone, is undergoing, or will undergo a hematopoietic stem cell transplant.

In some embodiments, the method comprises administering to a patient suffering from, or at risk for developing engraftment syndrome (ES) an amount of a compound of the disclosure in an amount sufficient to inhibit MASP-2 dependent complement activation in the mammalian subject to thereby treat the disease or disorder. In some embodiments, the subject suffering from, or at risk for developing ES has previously undergone, is undergoing, or will undergo a hematopoietic stem cell transplant.

In some embodiments, the method comprises administering to a patient suffering from, or at risk for developing fluid overload (FO) an amount of a compound of the disclosure in an amount sufficient to inhibit MASP-2 dependent complement activation in the mammalian subject to thereby treat the disease or disorder. In some embodiments, the subject suffering from, or at risk for developing FO has previously undergone, is undergoing, or will undergo a hematopoietic stem cell transplant.

In some embodiments, the method comprises administering to a patient suffering from any of the above-referenced diseases or conditions an amount of a compound as disclosed in PCT Application No. PCT/US19/34225, which is hereby incorporated by reference in its entirety.

In some embodiments, the MASP-2-dependent complement-associated disease or disorder is a renal condition. In some embodiments, the renal condition is selected from the group consisting of mesangioproliferative glomerulonephritis, membranous glomerulonephritis, membranoproliferative glomerulonephritis (mesangiocapillary glomerulonephritis), acute post infectious glomerulonephritis (poststreptococcal glomerulonephritis), C3 glomerulopathy, cryoglobulinemic glomerulonephritis, pauci-immune necrotizing crescentic glomerulonephritis, lupus nephritis, Henoch-Schonlein purpura nephritis, IgA nephropathy, or combinations thereof.

In some embodiments, the MASP-2-dependent complement-associated disease or disorder is renal fibrosis (e.g., tubulointerstitial fibrosis) and/or proteinuria in a subject suffering from or at risk for developing chronic kidney disease, chronic renal failure, glomerular disease (e.g., focal segmental glomerulosclerosis), an immune complex disorder (e.g., IgA nephropathy, membranous nephropathy), lupus nephritis, nephrotic syndrome, diabetic nephropathy, tubulointerstitial damage and glomerulonepthritis (e.g., C3 glomerulopathy), or a disease or condition associated with proteinuria, including, but not limited to, nephrotic syndrome, pre-eclampsia, eclampsia, toxic lesions of kidneys, amyloidosis, collagen vascular diseases (e.g., systemic lupus erythematosus), dehydration, glomerular diseases (e.g., membranous glomerulonephritis, focal segmental glomerulonephritis, C3 glomerulopathy, minimal change disease, lipoid nephrosis), strenuous exercise, stress, benign orthostatis (postural) proteinuria, focal segmental glomerulosclerosis, IgA nephropathy (i.e., Berger's disease), IgM nephropathy, membranoproliferative glomerulonephritis, membranous nephropathy, minimal change disease, sarcoidosis, Alport's syndrome, diabetes mellitus (diabetic nephropathy), drug-induced toxicity (e.g., NSAIDS, nicotine, penicillamine, lithium carbonate, gold and other heavy metals, ACE inhibitors, antibiotics (e.g., adriamycin), opiates (e.g., heroin), or other nephrotoxins), Fabry's disease, infections (e.g., HIV, syphilis, hepatitis A, B or C, poststreptococcal infection, urinary schistosomiasis), aminoaciduria, Fanconi syndrome, hypertensive nephrosclerosis, interstitial nephritis, sickle cell disease, hemoglobinuria, multiple myeloma, myoglobinuria, organ rejection (e.g., kidney transplant rejection), ebola hemorrhagic fever, Nail patella syndrome, familial Mediterranean fever, HELLP syndrome, systemic lupus erythematosus, Wegener's granulomatosis, Rheumatoid arthritis, Glycogen storage disease type 1, Goodpasture's syndrome, Henoch-Schonlein purpura, urinary tract infection which has spread to the kidneys, Sjogren's syndrome or post-infections glomerulonepthritis.

In some embodiments, the MASP-2-dependent complement-associated disease or disorder is selected from the group consisting of renal fibrosis, proteinuria, or combinations thereof.

In some embodiments, the MASP-2-dependent complement-associated disease or disorder is an inflammatory reaction resulting from tissue or solid organ transplantation, including allotransplantation or xenotransplantation of whole organs (e.g., kidney, heart, liver, pancreas, lung, cornea, and the like) or tissue grafts (e.g., valves, tendons, bone marrow, and the like).

In some embodiments, the MASP-2-dependent complement-associated disease or disorder is an ischemia reperfusion injury (I/R), including myocardial I/R, gastrointestinal I/R, renal I/R, and I/R following an aortic aneurism repair, I/R associated with cardiopulmonary bypass, cerebral I/R, stroke, organ transplant or reattachment of severed or traumatized limbs or digits, revascularization to transplants and/or replants, and hemodynamic resuscitation following shock, surgical procedures, or similar, or combinations thereof.

In some embodiments, the MASP-2-dependent complement-associated disease or disorder is a traumatic brain injury (TBI), including TBI associated with penetrating injuries or TBI associated with closed injuries (i.e., injuries that do not penetrate the brain). In some embodiments, the TBI results in one or more complications, including cognitive defects, personality changes, depression, anxiety, and/or post-traumatic stress disorder.

In some embodiments, the MASP-2-dependent complement-associated disease or disorder is a complication associated with non-obese diabetes (Type-1 diabetes or Insulin-dependent diabetes mellitus) and/or complications associated with Type-1 or Type-2 (adult onset) diabetes including diabetic angiopathy, diabetic neuropathy, diabetic retinopathy, diabetic macular edema, and the like, or combinations thereof.

In some embodiments, the MASP-2-dependent complement-associated disease or disorder is a cardiovascular disease or disorder, including Henoch-Schonlein purpura nephritis, systemic lupus erythematosus-associated vasculitis, vasculitis associated with rheumatoid arthritis (also called malignant rheumatoid arthritis), immune complex vasculitis, and Takayasu's disease; dilated cardiomyopathy; diabetic angiopathy; Kawasaki's disease (arteritis); venous gas embolus (VGE); and inhibition of restenosis following stent placement, rotational atherectomy, percutaneous transluminal coronary angioplasty (PTCA), and the like; or combinations thereof.

In some embodiments, the MASP-2-dependent complement-associated disease or disorder is an inflammatory gastrointestinal disorder, including pancreatitis, diverticulitis and bowel disorders including Crohn's disease, ulcerative colitis, irritable bowel syndrome, inflammatory bowel disease (IBD), or similar, or combinations thereof.

In some embodiments, the MASP-2-dependent complement-associated disease or disorder is a pulmonary disorder, including acute respiratory distress syndrome, transfusion-related acute lung injury, ischemia/reperfusion acute lung injury, chronic obstructive pulmonary disease, asthma, Wegener's granulomatosis, antiglomerular basement membrane disease (Goodpasture's disease), meconium aspiration syndrome, aspiration pneumonia, bronchiolitis obliterans syndrome, idiopathic pulmonary fibrosis, acute lung injury secondary to burn, non-cardiogenic pulmonary edema, transfusion-related respiratory depression, emphysema, and the like, or combinations thereof.

In some embodiments, the MASP-2-dependent complement-associated disease or disorder is an extracorporeal exposure-triggered inflammatory reaction and the method comprises treating a subject undergoing an extracorporeal circulation procedure. In some embodiments, the extracorporeal circulation procedure includes hemodialysis, plasmapheresis, leukopheresis, extracorporeal membrane oxygenation (ECMO), heparin-induced extracorporeal membrane oxygenation LDL precipitation (HELP), cardiopulmonary bypass (CPB), and the like, or combinations thereof. In some embodiments, the method further comprises treating a subject undergoing an extracorporeal circulation procedure.

In some embodiments, the MASP-2-dependent complement-associated disease or disorder is selected from inflammatory or non-inflammatory arthritides and other musculoskeletal disorders, e.g., osteoarthritis, rheumatoid arthritis, juvenile rheumatoid arthritis, gout, neuropathic arthropathy, psoriatic arthritis, ankylosing spondylitis or other spondyloarthropathies and crystalline arthropathies, muscular dystrophy, systemic lupus erythematosus (SLE), or similar, or combinations thereof.

In some embodiments, the MASP-2-dependent complement-associated disease or disorder is a skin disorder, for example, psoriasis, autoimmune bullous dermatoses, eosinophilic spongiosis, bullous pemphigoid, epidermolysis bullosa acquisita, atopic dermatitis, herpes gestationis, and other skin disorders. In some embodiments, the MASP-2-dependent complement-associated disease or disorder is a thermal burn, chemical burn, or combinations thereof, including capillary leakage caused thereby.

In some embodiments, the MASP-2-dependent complement-associated disease or disorder is a peripheral nervous system (PNS) and/or central nervous system (CNS) disorder or injury including multiple sclerosis (MS), myasthenia gravis (MG), Huntington's disease (HD), amyotrophic lateral sclerosis (ALS), Guillain Barre syndrome, reperfusion following stroke, degenerative discs, cerebral trauma, Parkinson's disease (PD), Alzheimer's disease (AD), Miller-Fisher syndrome, cerebral trauma and/or hemorrhage, traumatic brain injury, demyelination, meningitis, or similar, or combinations thereof.

In some embodiments, the MASP-2-dependent complement-associated disease or disorder is sepsis or a condition resulting from sepsis including severe sepsis, septic shock, acute respiratory distress syndrome resulting from sepsis, hemolytic anemia, systemic inflammatory response syndrome, hemorrhagic shock, or the like, or combinations thereof.

In some embodiments, the MASP-2-dependent complement-associated disease or disorder is a urogenital disorder including painful bladder disease, sensory bladder disease, chronic abacterial cystitis and interstitial cystitis, male and female infertility, placental dysfunction and miscarriage, pre-eclampsia, or similar, as well as combinations thereof. In some embodiments, the MASP-2-dependent complement-associated disease or disorder is an inflammatory reaction in a subject being treated with chemotherapeutics, radiation therapy, or combinations thereof.

In some embodiments, the MASP-2-dependent complement-associated disease or disorder is an inflammatory reaction in a subject being treated with chemotherapeutics and/or radiation therapy, including for the treatment of cancerous conditions.

In some embodiments, the MASP-2-dependent complement-associated disease or disorder is an angiogenesis-dependent cancer, including a solid tumor(s), blood borne tumor(s), high-risk carcinoid tumors, tumor metastases, and the like, or combinations thereof.

In some embodiments, the MASP-2-dependent complement-associated disease or disorder is an angiogenesis-dependent benign tumor, including hemangiomas, acoustic neuromas, neurofibromas, trachomas, carcinoid tumors, pyogenic granulomas, or similar, or combinations thereof.

In some embodiments, the MASP-2-dependent complement-associated disease or disorder is an endocrine disorder including Hashimoto's thyroiditis, stress, anxiety, other potential hormonal disorders involving regulated release of prolactin, growth or insulin-like growth factor, adrenocorticotropin from the pituitary, or similar, or combinations thereof.

In some embodiments, the MASP-2-dependent complement-associated disease or disorder is an ophthalmic disease or disorder including age-related macular degeneration, glaucoma, endophthalmitis, and the like, or combinations thereof.

In some embodiments, the MASP-2-dependent complement-associated disease or disorder is an ocular angiogenic disease or disorder including age-related macular degeneration, uveitis, ocular melanoma, corneal neovascularization, primary pterygium, HSV stromal keratitis, HSV-1-induced corneal lymphangiogenesis, proliferative diabetic retinopathy, diabetic macular edema, retinopathy of prematurity, retinal vein occlusion, corneal graft rejection, neovascular glaucoma, vitreous hemorrhage secondary to proliferative diabetic retinopathy, neuromyelitis optica, rubeosis, or similar, or combinations thereof.

In some embodiments, the MASP-2-dependent complement-associated disease or disorder is disseminated intravascular coagulation (DIC) or other complement mediated coagulation disorder, including DIC secondary to sepsis, severe trauma, including neurological trauma (e.g., acute head injury; see Kumura et al, Acta Neurochirurgica 55:23-28 (1987), infection (e.g., bacterial, viral, fungal, parasitic), cancer, obstetrical complications, liver disease, severe toxic reaction (e.g., snake bite, insect bite, transfusion reaction), shock, heat stroke, transplant rejection, vascular aneurysm, hepatic failure, cancer treatment by chemotherapy or radiation therapy, burn, or accidental radiation exposure.

In some embodiments, the MASP-2-dependent complement-associated disease or disorder is selected from the group consisting of acute radiation syndrome, dense deposit disease, Degos Disease, Catastrophic Antiphospholipid Syndrome (CAPS), Behcet's disease, cryoglobulinemia, paroxysmal nocturnal hemoglobinuria (“PNH”), cold agglutinin disease, or combinations thereof.

In some embodiments, the MASP-2-dependent complement-associated disease or disorder is atypical hemolytic uremic syndrome (aHUS).

In some embodiments, the MASP-2-dependent complement-associated disease or disorder is hematopoietic stem cell transplant-associated TMA.

In some embodiments, the MASP-2-dependent complement-associated disease or disorder is immunoglobulin A nephropathy (IgAN).

In some embodiments, the MASP-2-dependent complement-associated disease or disorder is lupus nephritis (LN).

In some embodiments, the MASP-2-dependent complement-associated disease or disorder is COVID-19 induced acute respiratory distress syndrome (ARDS) or COVID-19 induced pneumonia.

In some embodiments, the method comprises administering to a patient suffering from, or at risk for developing a disease, disorder or condition associated with fibrin-induced activation of the complement system and the associated activation of the coagulation and/or contact systems an amount of a compound according to any one of the foregoing embodiments (e.g., a compound of Formulae (I), (I-A), (I-B), (I-C), (II), (III), (IV), (V), (V-A), (V-B), and embodiments thereof) in an amount sufficient to inhibit MASP-2 dependent complement activation in the mammalian subject to thereby treat the disease or disorder.

In some embodiments, the subject is suffering from, or at risk of developing, a disease, disorder or condition associated with complement-related inflammation, excessive coagulation or contact system activation initiated by fibrin or activated platelets. In some embodiments, the subject is suffering from a disease or disorder selected from the group consisting of arterial thrombosis, venous thrombosis, deep vein thrombosis, post-surgical thrombosis, restenosis following coronary artery bypass graft and/or an interventional cardiovascular procedure (e.g., angioplasty or stent placement), atherosclerosis, plaque rupture, plaque instability, restenosis, hypotension, acute respiratory distress syndrome (ARDS), systemic inflammatory response syndrome (SIRS), disseminated intravascular coagulation (DIC), veno-occlusive disease (VOD), thrombotic microangiopathy, lupus nephritis, superficial thrombophlebitis, Factor V Leiden mutation, ischemic/reperfusion injury, human immunodeficiency virus (HIV) infection, undergoing hormone-replacement therapy (HRT), Alzheimer's disease and/or suffering from a hypercoagulable state.

In some embodiments, the subject is suffering from, or at risk for developing an acquired hypercoagulable state due to at least one or more of the following: undergoing therapy with a drug selected from the group consisting of 5-FU, GM-CSF, cisplatin, heparin, COX-2 inhibitor, contrast media, corticosteroids and antipsychotics; venous stasis (immobilization, surgery, etc.), antiphospholipid syndrome, cancer (promyelocytic leukemia, lung, breast, prostate, pancreas, stomach and colon tumors), tissue injury due to trauma or surgery, presence of a catheter in a central vein, acquired deficiency of a protein involved in clot formation (e.g., protein C), paroxysmal nocturnal hemoglobinuria (PNH), elevated levels of homocysteine, heart failure, presence of a mechanical valve, pulmonary hypertension with in situ thrombosis, atrial fibrillation, heparin-induced thrombocytopenia (HIT), heparin-induced thrombocytopenia and thrombosis (HITT), Kawasaki disease with in situ thrombus, Takayasu arteritis with in situ thrombus, thrombophilia of metastatic cancer, elevated Factor VIII levels, pregnancy, inflammatory bowel disease (IBD), or due to a genetic defect that causes or increases the risk of developing, a hypercoagulable state, such as a genetic defect selected from the group consisting of a Prothrombin 20210 gene mutation, an MTHFR mutation, a deficiency of protein C, a deficiency of protein S, a deficiency of protein A, a deficiency of protein Z, an antithrombin deficiency, and a genetic disorder producing thrombophilia.

In some embodiments, the subject is suffering from, or at risk for developing, a disease or disorder that is amenable to treatment with a kallikrein inhibitor. In some embodiments, the subject is suffering from, or at risk for developing a disease or disorder amenable to treatment with a kallikrein inhibitor is selected from the group consisting of hereditary angioedema, diabetic macular edema and bleeding during cardiopulmonary bypass. In some embodiments, the subject is suffering from, or at risk for developing, a disease or disorder that is amenable to treatment with a thrombin inhibitor, such as arterial thrombosis, venous thrombosis, pulmonary embolism, atrial fibrillation, heparin-induced thrombocytopenia, conversion from one anticoagulant to another, or off-label use for extracorporeal circuit patency of continuous renal replacement therapy (CRRT) in critically ill patients with HIT (maintenance).

In some embodiments, the subject has previously experienced, is currently suffering from, or is at risk for developing atrial fibrillation and the MASP-2 inhibitory compound (e.g., a compound of Formulae (I), (I-A), (I-B), (I-C), (II), (III), (IV), (V), (V-A), (V-B), and embodiments thereof) is administered in an amount sufficient to reduce the risk of stroke in said subject. In some embodiments, the subject is suffering from, or at risk for developing, a disease or disorder that is amenable to treatment with a factor XII inhibitor, such as deep vein thrombosis (both primary prophylaxis and extended therapy), pulmonary embolism, nonvalvular atrial fibrillation, prevention of recurrent ischemia after acute coronary syndrome in subjects with or without atrial fibrillation, end-stage renal disease, cerebral ischemia, angina, or to reduce or prevent clotting associated with medical devices (e.g., valves, small caliber grafts, etc.) and/or extracorporeal circuits.

In some embodiments, the subject has previously experienced, is currently suffering from, or is at risk for developing nonvalvular atrial fibrillation and the MASP-2 inhibitory compound (e.g., a compound of Formulae (I), (I-A), (I-B), (I-C), (II), (III), (IV), (V), (V-A), (V-B), and embodiments thereof) is administered in an amount sufficient to reduce the risk of stroke and/or embolism in said subject. In some embodiments, the subject has an acquired disease or disorder that increases the propensity for thromboembolism, such as a disease or disorder selected from the group consisting of atherosclerosis, antiphospholipid antibodies, cancer (e.g., promyelocytic leukemia, lung, breast, prostate, pancreatic, stomach and colon), hyperhomocysteinemia, infection, tissue injury, venous stasis (such as due to surgery, orthopedic or paralytic immobilization, heart failure, pregnancy, or obesity) and a subject taking oral contraceptives that contain estrogen.

In some embodiments, the subject is in need of anticoagulant therapy and the MASP-2 inhibitory compound (e.g., a compound of Formulae (I), (I-A), (I-B), (I-C), (II), (III), (IV), (V), (V-A), (V-B), and embodiments thereof) is used as a replacement for standard anticoagulant therapy (e.g., Warfarin). In some embodiments, the subject has a condition that normally prohibits standard anticoagulant therapy, such as CNS amyloid angiopathy. In some embodiments of the method, the MASP-2 inhibitory compound is administered as a bridging agent perioperatively in a subject otherwise on standard anticoagulation therapy. In some embodiments, the subject has sickle cell disease which is a vaso-occlusive disorder involving activation of platelets.

Atypical hemolytic uremic syndrome (aHUS) is part of a group of conditions termed “Thrombotic microangiopathies.” In the atypical form of HUS (aHUS), the disease is associated with defective complement regulation and can be either sporadic or familial. Familial cases of aHUS are associated with mutations in genes coding for complement activation or complement regulatory proteins, including complement factor H, factor I, factor B, membrane cofactor CD46 as well as complement factor H-related protein 1 (CFHR1) and complement factor H-related protein 3 (CFHR3). (Zipfel, P. F., et al., PloS Genetics 3(3):e41 (2007)). The unifying feature of this diverse array of genetic mutations associated with aHUS is a predisposition to enhanced complement activation on cellular or tissue surfaces. A subject is a risk for developing aHUS upon the onset of at least one or more symptoms indicative of aHUS (e.g., the presence of anemia, thrombocytopenia and/or renal insufficiency) and/or the presence of thrombotic microangiopathy in a biopsy obtained from the subject. The determination of whether a subject is at risk for developing aHUS comprises determining whether the subject has a genetic predisposition to developing aHUS, which may be carried out by assessing genetic information (e.g. from a database containing the genotype of the subject), or performing at least one genetic screening test on the subject to determine the presence or absence of a genetic marker associated with aHUS (i.e., determining the presence or absence of a genetic mutation associated with aHUS in the genes encoding complement factor H (CFH), factor I (CFI), factor B (CFB), membrane cofactor CD46, C3, complement factor H-related protein 1 (CFHR1), or THBD (encoding the anticoagulant protein thrombodulin) or complement factor H-related protein 3 (CFHR3), or complement factor H-related protein 4 (CFHR4)) either via genome sequencing or gene-specific analysis (e.g., PCR analysis), and/or determining whether the subject has a family history of aHUS. Methods of genetic screening for the presence or absence of a genetic mutation associated with aHUS are well established, for example, see Noris M et al. “Atypical Hemolytic-Uremic Syndrome,” 2007 Nov. 16 [Updated 2011 Mar. 10]. In: Pagon R A, Bird T D, Dolan C R, et al., editors. GeneReviews™, Seattle (WA): University of Washington, Seattle.

Hematopoietic stem cell transplant-associated TMA (HSCT-TMA) is a life-threatening complication that is triggered by endothelial injury. The kidney is the most commonly affected organ, though HSCT-TMA can be a multi-system disease that also involves the lung, bowel, heart, and brain. The occurrence of even mild TMA is associated with long-term renal impairment. Development of post-allogeneic HSCT-associated TMA differs in frequency based on varying diagnostic criteria and conditioning and graft-versus-host disease prophylaxis regimens, with calcineurin inhibitors being the most frequent drugs implicated (Ho V T et al., Biol Blood Marrow Transplant, 11(8):571-5, 2005).

Immunoglobulin A nephropathy (IgAN) is an autoimmune kidney disease resulting in intrarenal inflammation and kidney injury. IgAN is the most common primary glomerular disease globally. With an annual incidence of approximately 2.5 per 100,000, it is estimated that 1 in 1400 persons in the U.S. will develop IgAN. As many as 40% of patients with IgAN will develop end-stage renal disease (ESRD). Patients typically present with microscopic hematuria with mild to moderate proteinuria and variable levels of renal insufficiency (Wyatt R. J., et al., NEnglJ Med 36S(25):2402-4, 2013). Clinical markers such as impaired kidney function, sustained hypertension, and heavy proteinuria (over 1 g per day) are associated with poor prognosis (Goto M et al., Nephrol Dial Transplant 24(10):3068-74, 2009; Berthoux F. et al., J Am Soc Nephrol 22(4):752-61, 2011). Proteinuria is the strongest prognostic factor independent of other risk factors in multiple large observational studies and prospective trials (Coppo R. et al., J Nephrol 18(5):503-12, 2005; Reich H. N., et al., J Am Soc Nephrol 18(12):3177-83, 2007). It is estimated that 15-20% of patients reach ESRD within 10 years of disease onset if left untreated (D'Amico G., Am J Kidney Dis 36(2):227-37, 2000). The diagnostic hallmark of IgAN is the predominance of IgA deposits, alone or with IgG, IgM, or both, in the glomerular mesangium.

A main complication of systemic lupus erythematosus (SLE) is nephritis, also known as lupus nephritis, which is classified as a secondary form of glomerulonephritis. Up to 60% of adults with SLE have some form of kidney involvement later in the course of the disease (Koda-Kimble et al., Koda-Kimble and Young's Applied Therapeutics: the clinical use of drugs, 10th Ed, Lippincott Williams & Wilkins: pages 792-9, 2012) with a prevalence of 20-70 per 100,000 people in the U.S. Lupus nephritis often presents in patients with other symptoms of active SLE, including fatigue, fever, rash, arthritis, serositis, or central nervous system disease (Pisetsky D. S. et al., Med Clin North Am 81(1): 113-28, 1997). Some patients have asymptomatic lupus nephritis; however, during regular follow-up, laboratory abnormalities such as elevated serum creatinine levels, low albumin levels, or urinary protein or sediment suggest active lupus nephritis.

In some embodiments, the MASP-2-dependent complement-associated disease or disorder is a traumatic brain injury (TBI). TBI may involve penetration of the brain substance (“penetrating” injuries) or injuries that do not penetrate the brain (“closed” injuries), and can result in long-term or lifelong physical, cognitive, behavioral, and emotional consequences. (Langlois, J. A. et al., J. Head Trauma Rehabil. 21:375-378, 2006). TBI can lead to changes in several neurotransmitter systems, including release of glutamate and other excitatory amino acids during the acute phase and chronic alterations in the catecholaminergic and cholinergic systems, which may be associated with neurobehavioral disability (McAllister, T. W. Dialogues Clin. Neurosci. 13:287-300, 2011). Survivors of significant TBI often suffer from cognitive defects, personality changes, and increased psychiatric disorders, particularly depression, anxiety, and post-traumatic stress disorder. Despite intense research, no clinically effective treatment for TBI that can reduce mortality and morbidity and improve functional outcome has yet to be found.

### VI. Compositions, Dosage, and Administration

The compounds as described herein (e.g., a compound of Formulae (I), (I-A), (I-B), (I-C), (II), (III), (IV), (V), (V-A), (V-B), and embodiments thereof) can be administered in a manner compatible with the dosage formulation, and in such amount as will be effective or suitable for treatment. The quantity to be administered depends on a variety of factors, including, e.g., the age, body weight, physical activity, and diet of the individual, and the desired effect. In certain embodiments, the size of the dose may also be determined by the existence, nature, and extent of any adverse side effects that accompany the administration of the compound in a particular individual.

It will be understood, however, that the specific dose level and frequency of dosage for any particular patient may be varied by a physician and will depend upon a variety of factors, including the activity of the specific compound employed, the metabolic stability and length of action of that compound, the age, body weight, hereditary characteristics, general health, sex, diet, mode, and time of administration, rate of excretion, drug combination, the severity of the particular condition, and the host undergoing therapy.

In certain embodiments, the dose may take the form of solid, semi-solid, liquid, or gas forms. In certain embodiments, the unit dosage form is suitable for ease of administration and uniformity of dosage.

As used herein, the term “unit dosage form” refers to physically discrete units suitable as unitary dosages for humans and other mammals, suited as unitary dosages for the subject to be treated, with each unit containing a predetermined quantity of an active agent calculated to produce the desired onset, tolerability, efficacious and/or therapeutic effects, in association with a suitable pharmaceutical excipient (e.g., an ampoule). In addition, more concentrated dosage forms may be prepared, from which the more dilute unit dosage forms may then be produced.

The compounds described herein (e.g., a compound of Formulae (I), (I-A), (I-B), (I-C), (II), (III), (IV), (V), (V-A), (V-B), and embodiments thereof) can be administered to a subject in need of treatment using methods known in the art, such as by oral administration or by injection. The injection can be, e.g., subcutaneous, intravenous, intraperitoneal, or intramuscular. As described herein, parenteral formulations can be prepared in a unit dosage form.

The pharmaceutical compositions of the present application comprise a therapeutically effective amount of a compound of the present disclosure ((e.g., a compound of Formulae (I), (I-A), (I-B), (I-C), (II), (III), (IV), (V), (V-A), (V-B), and embodiments thereof) formulated together with one or more pharmaceutically acceptable carriers or excipient. As used herein, the term “pharmaceutically acceptable carrier” means a non-toxic, inert solid, semi-solid or liquid filler, diluent, encapsulating material or formulation auxiliary of any type. The pharmaceutical compositions of this application can be administered to humans and other animals orally, rectally, parenterally, intracistemally, intravaginally, intraperitoneally, topically (as by powders, ointments, or drops), buccally, or as an oral or nasal spray.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations include, for example, sterile injectable aqueous or oleaginous suspensions formulated according to the known art using suitable dispersing or wetting agents and suspending agents. The sterile injectable preparation may also be a sterile injectable solution, suspension, or emulsion in a nontoxic parenterally acceptable diluent or solvent. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

To prolong the effect of a compound of Formulae (I), (I-A), (I-B), (I-C), (II), (III), (IV), (V), (V-A), (V-B), and embodiments thereof, it can be desirable to slow the absorption of the compound from subcutaneous or intramuscular injection. This may be accomplished using a liquid suspension of crystalline or amorphous material with poor water solubility. The rate of absorption of the drug then depends upon its rate of dissolution which, in turn, may depend upon, for example, crystal size and crystalline form. Alternatively, delayed absorption of a parenterally administered compound form is accomplished by dissolving or suspending the compound in an oil vehicle.

Solid compositions of a similar type may also be employed as fillers in soft and hard filled gelatin capsules using such excipients as lactose or milk sugar as well as high molecular weight polyethylene glycols, and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art.

In such solid dosage forms, the active compound may be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents.

Dosage forms for topical or transdermal administration of a compound as disclosed herein (e.g., a compound of Formulae (I), (I-A), (I-B), (I-C), (II), (III), (IV), (V), (V-A), (V-B), and embodiments thereof) include ointments, pastes, creams, lotions, gels, powders, solutions, sprays, inhalants, or patches. For example, the active component may be ad-mixed under sterile conditions with a pharmaceutically acceptable carrier or excipient, and any needed preservatives or buffers as may be required.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present disclosure, disorders are treated or prevented in a subject, such as a human or other animal, by administering to the subject a therapeutically effective amount of a compound of Formulae (I), (I-A), (I-B), (I-C), (II), (III), (IV), (V), (V-A), (V-B), and embodiments thereof, according to any one of the foregoing embodiments, in such amounts and for such time as is necessary to achieve the desired result. As is well understood in the medical arts, a therapeutically effective amount of a compound of Formulae (I), (I-A), (I-B), (I-C), (II), (III), (IV), (V), (V-A), (V-B), and embodiments thereof, will be at a reasonable benefit/risk ratio applicable to any medical treatment.

In general, compounds (e.g., a compound of Formulae (I), (I-A), (I-B), (I-C), (II), (III), (IV), (V), (V-A), (V-B), and embodiments thereof) will be administered in therapeutically effective amounts via any of the usual and acceptable modes known in the art, either singly or in combination with one or more other therapeutic agents. A therapeutically effective amount may vary widely depending on the severity of the disease, the age and relative health of the subject, the potency of the compound used, and other factors.

In general, satisfactory results are indicated to be obtained systemically at daily dosages of from about 0.03 to 2.5 mg/kg of body weight. An indicated daily dosage in the larger mammal, e.g., humans, is in the range from about 0.5 mg to about 250 mg, about 5 mg to about 150 mg, about 5 mg to about 100 mg, about 10 mg to about 75 mg, about 10 mg to about 50 mg, about 10, about 20, about 30, about 40, or about 50 mg, conveniently administered, e.g., in divided doses up to four times a day or in retard form. Suitable unit dosage forms for oral administration comprise from ca. 1 to 60 mg active ingredient.

In certain embodiments, a therapeutic amount or dose of the compound (e.g., compounds of Formulae (I), (I-A), (I-B), (I-C), (II), (III), (IV), (V), (V-A), (V-B), and embodiments thereof) may range from about 0.1 mg/kg to about 500 mg/kg, alternatively from about 1 mg/kg to about 50 mg/kg. In general, treatment regimens according to the present application comprise administration to a patient in need of such treatment from about 10 mg to about 1000 mg of the compound(s) per day in single or multiple doses. Therapeutic amounts or doses will also vary depending on route of administration, as well as the possibility of co-usage with other agents.

Upon improvement of a subject's condition, a maintenance dose of a compound, composition or combination of this disclosure may be administered, if necessary.

Subsequently, the dosage or frequency of administration, or both, may be reduced, as a function of the symptoms, to a level at which the improved condition is retained when the symptoms have been alleviated to the desired level, or treatment may cease. The subject may, however, require intermittent treatment on a long-term basis upon any recurrence of disease symptoms.

It will be understood, however, that the total daily usage of the compounds (e.g., a compound of Formulae (I), (I-A), (I-B), (I-C), (II), (III), (IV), (V), (V-A), (V-B), and embodiments thereof), will be decided by an attending physician within the scope of sound medical judgment. The specific inhibitory dose for any particular patient will depend upon a variety of factors, including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

This disclosure also provides for a pharmaceutical combination, e.g., a kit, comprising:
a) a first agent which is a compound of Formulae (I), (I-A), (I-B), (I-C), (II), (III), (IV), (V), (V-A), (V-B), and embodiments thereof, as disclosed herein, in free form or in a pharmaceutically acceptable salt form, and
b) at least one co-agent.

The kit can comprise instructions for its administration.

Methods for preparing such dosage forms are known to those skilled in the art (see, e.g., REMINGTON'S PHARMACEUTICAL SCIENCES, 18th ED., Mack Publishing Co., Easton, PA (1990)). The dosage forms typically include a conventional pharmaceutical carrier or excipient, and may additionally include other medicinal agents, carriers, adjuvants, diluents, tissue permeation enhancers, solubilizers, and the like. Appropriate excipients can be tailored to the particular dosage form and route of administration, by methods well known in the art (see, e.g., REMINGTON'S PHARMACEUTICAL SCIENCES, 18th ED., Mack Publishing Co., Easton, PA (1990)).

### EXAMPLES

The following examples are provided by way of illustration only and not by way of limitation. Those of skill in the art will readily recognize a variety of noncritical parameters which could be changed or modified to yield essentially similar results.

### Example 1

### General Methods

Compounds described herein, including salts thereof, can be prepared using known organic synthesis techniques and can be synthesized according to any of numerous possible synthetic routes, such as those illustrated in the Examples.

The reactions for preparing compounds described herein can be carried out in suitable solvents which can be readily selected by one of skill in the art of organic synthesis. Suitable solvents can be substantially non-reactive with the starting materials or reactants, the intermediates, or the products, at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the disclosure can involve the protection and deprotection of various chemical groups. The need for protection and deprotection, and the selection of appropriate protecting groups, can be readily determined by one skilled in the art. The chemistry of protecting groups is described, in e.g., Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6th Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high-performance liquid chromatography (HPLC) or thin layer chromatography (TLC).

The particular synthetic methods used in the Examples provide general guidance in connection with preparing the compounds of the disclosure. One skilled in the art would understand that the preparations can be modified or optimized using general knowledge of organic chemistry to prepare various compounds within the scope of the present disclosure.

Starting materials, reagents, and intermediates whose synthesis is not described herein are either commercially available, known in the literature, or may be prepared by methods known to one skilled in the art.

It will be appreciated by one skilled in the art that the processes described herein are not the exclusive means by which compounds of the disclosure may be synthesized, and that a broad repertoire of synthetic organic reactions is available to be potentially employed in synthesizing compounds of the disclosure. The person skilled in the art knows how to select and implement appropriate synthetic routes. Suitable synthetic methods of starting materials, intermediates, and products may be identified by reference to the literature, including reference sources such as: Advances in Heterocyclic Chemistry, Vols. 1-107 (Elsevier, 1963-2012); Journal of Heterocyclic Chemistry, Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2nd Edition, 2004); Katritzky et al. (Ed.), Comprehensive Heterocyclic Chemistry (Pergamon Press, 1984); Katritzky et al., Comprehensive Heterocyclic Chemistry II (Pergamon Press, 1996); Smith et al., March's Advanced Organic Chemistry: Reactions, Mechanisms, and Structure, 6th Ed. (Wiley, 2007); Trost et al. (Ed.), Comprehensive Organic Synthesis (Pergamon Press, 1991).

If not otherwise stated, chromatography refers to flash chromatography conducted on silica gel. “Amine column” refers to flash chromatography conducted on Redisep Rf Gold® high performance amine column. HPLC purification refers to high performance liquid chromatography performed by one of two methods. HPLC Method 1: A Gilson preparative reverse phase HPLC system with the combination of UV/ELS detectors (254 nm and 280 nm) and ThermoFisher Hypersil GOLD™ Agilent (21.2×250 mm) 5 μm C18 column; eluents consisted of a mixture of water and acetonitrile (with 0.05% trifluoroacetic acid); flow rate was typically 20 mL/min with a linear gradient of acetonitrile in water from 2-90% acetonitrile over 45 min; and the injection volume ranged from 1 to 3 mL, with a maximum 20 mg sample per injection. Method 2: A Waters™ preparative reverse phase HPLC system with the combination of UV/MS detectors (254 nm and 280 nm) and XBridge Prep (19×50 mm) C18 10 μM OBD column; eluents consisted of a mixture of water and acetonitrile (with 0.05% trifluoroacetic acid); flow rate was typically 50 mL/min with a linear gradient of acetonitrile in water from 5-95% acetonitrile over 8 min; and the injection volume ranged from 0.2 to 1 mL with a maximum 20 mg sample per injection.

### Abbreviations

μ micro
° C. degrees Celsius
ACN acetonitrile
anhyd anhydrous
aq aqueous
atm atmosphere(s)
ave average
Boc tert-butoxycarbonyl
CPME cyclopentyl methyl ether
conc concentration/concentrated
cmp no compound number
DCC N, N′-dicyclohexylcarbodiimide
DCM dichloromethane
DIEA N,N-diisopropylethylamine
DMA dimethylacetamide
DME dimethoxyethane
DMF dimethylformamide
DMSO dimethylsulfoxide
ES electrospray
EtOAc ethyl acetate
EtOH ethanol
g gram(s)
h hour(s)
HATU N-[(dimethylamino)-1H-1,2,3-triazolo-[4,5-b]pyridin-1-ylmethylene]-N-methylmethanaminium hexafluorophosphate N-oxide
HBTU N,N,N′,N′-tetramethyl-O-(1H-benzotriazol-1-yl)uronium hexafluorophosphate
HPLC high-performance liquid chromatography
L liter(s)
LiHMDS lithium bis(trimethylsilyl)amide
LiOH lithium hydroxide
m milli
M molar
MeCN acetonitrile
MeOH methanol
min minutes(s)
mL milliliter
mmol millimole
mol mole; molecular (as in mol wt)
MS mass spectrometry
N normal
NHS N-hydroxysuccinimide
NMP N-methyl-2-pyrrolidone
o ortho
obs'd observed
p para
Pd-PEPPSI-Ipent [1,3-bis(2,6-Di-3-pentylphenyl)imidazol-2-ylidene](3-chloropyridyl)palladium(II) dichloride
PdTrixiePhos G3 mesyl(2-(di-tert-butylphosphino)-1,1′-binaphthyl)[2-(2′-amino-1,1′-biphenyl)]palladium(II)
Ph phenyl
prep preparatory
psi pounds per square inch
PTFE polytetrafluoroethylene
sat saturated
temp temperature
TES triethylsilane
Tf triflate
TFA trifluoroacetic acid
THF tetrahydrofuran
TMSCN trimethylsilyl cyanide
p-TsOH para-toluenesulfonicacid

### General Chloro Displacement Procedure A

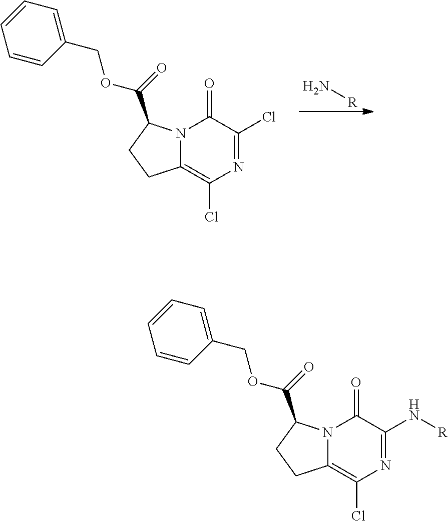

Benzyl (S)-1,3-dichloro-4-oxo-4,6,7,8-tetrahydropyrrolo[1,2-a]pyrazine-6-carboxylate (1 eq), amine (1.2-1.4 eq), and DIEA (2-4 eq) was added to ACN (volume used so that the carboxylate was 0.2-0.4 M in ACN). The reaction mixture was heated up to 70° C.-85° C. and allowed to stir until the reaction was complete. The crude material was concentrated down, loaded onto a silica gel column, and eluted with a heptane-EtOAc gradient. Pure fractions were collected and concentrated to afford the pure product.

### General Hydrogenolysis Procedure A

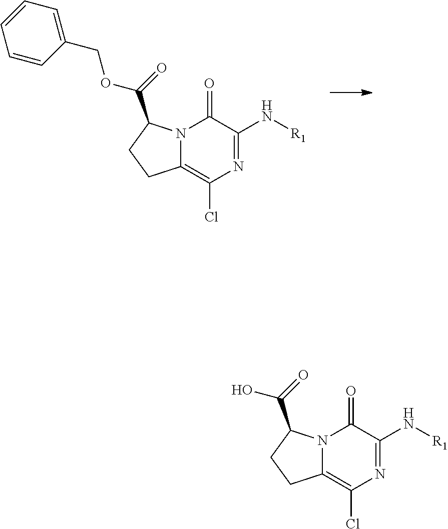

EtOH (volume used so that carboxylate was 0.1-0.4 M) was added to a reaction flask. The flask was evacuated and filled with N₂ gas. 10 mol % Pd/C (0.05 to 0.10 eq) and carboxylate (1 eq) were added. The flask was evacuated and backfilled with H₂ gas. Repeated 3 times. The reaction was stirred at room temperature under a blanket of H₂ gas until the reaction was complete. The reaction mixture was filtered through a PTFE micro filter and concentrated to afford the product.

### General Hydrogenolysis Procedure B

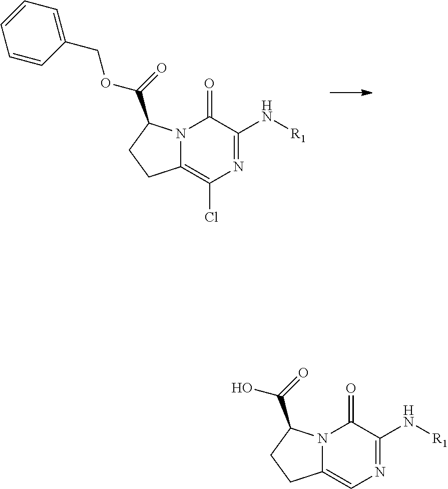

EtOH (volume used so that the concentration of carboxylate was 0.1-0.4 M) was added to a reaction flask. The flask was evacuated and filled with N₂ gas. 10 mol % Pd/C (0.05 to 0.10 eq), carboxylate (1 eq), and DIEA (2 eq) were added. The flask was evacuated and backfilled with H₂ gas. Repeated 3 times. The reaction was stirred at room temperature under a blanket of H₂ gas until the reaction was complete. The crude material was filtered through a PTFE micro filter and concentrated to afford the product.

### General Saponification Procedure A

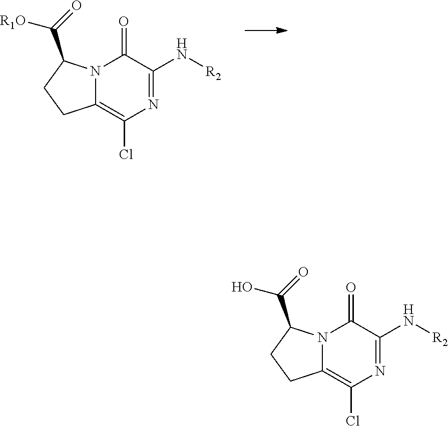

A solution of the ester was stirred in THF/MeOH (2:1) containing LiOH (5 eq) and H₂O (1 part equal to volume of MeOH) until the reaction was complete. The solution was evaporated to dryness, then H₂O (4 parts) was added to dissolve the residue. The pH was adjusted to approximately 1-2 with the dropwise addition of conc. aqueous HCl. The product generally precipitated out of solution and was collected by filtration, washed with H₂O and dried to give the product which was used as-is in the next synthetic step. In cases where the product did not precipitate out of solution, the product was extracted from the acidic aqueous layer with DCM, dried over Na₂SO₄ and conc in vacuo to give an oil which was used as-is in the next synthetic step. As known in the literature, base-catalyzed hydrolysis of esters can result in epimerization of a stereocenter alpha to the carbonyl.

### General Raney®-Ni Hydrogenation Procedure

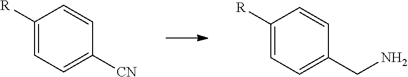

To a solution of cyano compound (1 eq) in NH₃-MeOH (7M, 100 eq), Raney® nickel (0.1 eq) was added. The reaction mixture was evacuated and charged with Ar, then evacuated and charged with H₂ gas to 1 atm for 12 h at 21° C. The reaction mixture was filtered through Celite© and rinsed with a mixture of MeOH and CH₂Cl₂, and the filtrate concentrated. The concentrated filtrate was purified on a reverse phase prep column to isolate the desired product.

### General Amide Coupling Procedure A

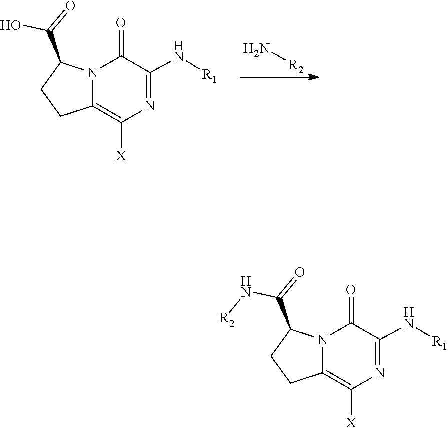

The carboxylic acid (1 eq), amine (1.2-1.4 eq), HATU (1.3-1.5 eq), and DIEA (3-4 eq) were dissolved in DMF (volume used so that the carboxylic acid was 0.1-0.4 M in DMF). The mixture was stirred at room temperature until the reaction was complete. The product was purified on an amine column eluting with heptane and then a DCM-MeOH gradient. The isolated material was then further purified on a silica gel column and eluted with a DCM-MeOH gradient. The eluent was concentrated to yield the product.

### General Amide Coupling Procedure B

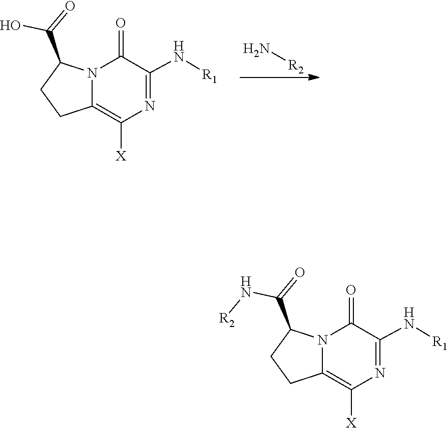

The carboxylic acid (1 eq), NHS (1.1 eq), DCC (1.1 eq), and amine (1.2 eq) was dissolved in DMF (volume used so that the carboxylic acid was 0.1-0.4 M in DMF). The mixture was stirred at room temperature until the reaction was complete. The mixture was concentrated, redissolved in 5% MeOH—CH₂Cl₂, and filtered to remove solids. The filtrate was concentrated and purified on a silica gel column, eluting with a 4-6% MeOH (7 M NH₃)—CH₂Cl₂ gradient. The eluent was concentrated to yield the product.

### General Boc Removal Procedure A

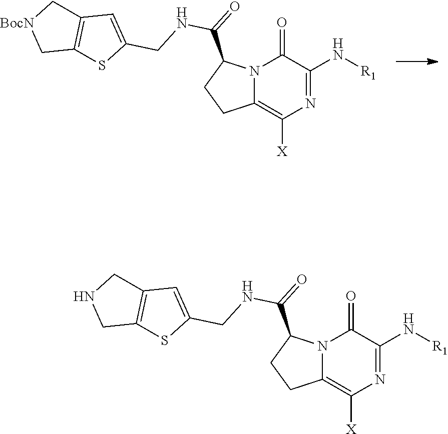

The starting material (1 eq) was dissolved in TFA (15-20 eq) in DCM (0.1-0.4 M). The mixture was stirred at room temperature until the reaction was complete. The product was purified on an amine column and eluted with a DCM-MeOH gradient. The eluent was concentrated to yield the product.

### General Suzuki-Miyaura Reaction Procedure A

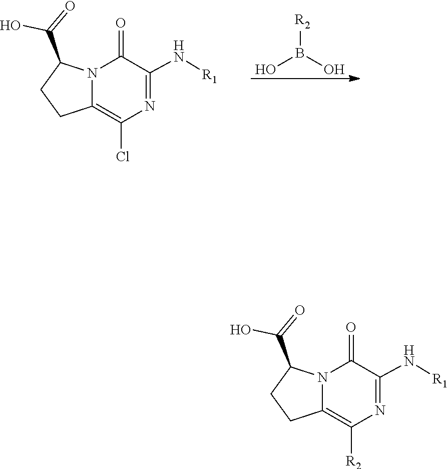

The chloride (1 eq), boronic acid (1.3 eq), K₂CO₃ (1.6 eq), and Pd-PEPPSI-Ipent (4 mol %) and EtOH (1 mL) were added to a microwave vial. The mixture was heated at 100° C. in a microwave synthesizer until the reaction was complete. Water was added and the mixture was washed with DCM 3 times. The aqueous layer was acidified with 2 N HCl to pH 2-3. The aqueous solution was extracted with chloroform 5 times. The organic layers were combined, washed with brine, dried over Na₂SO₄, filtered, and the filtrate concentrated to afford the product.

### General Suzuki-Miyaura Reaction Procedure B

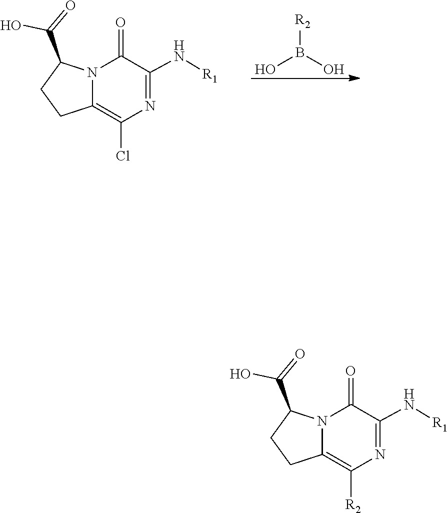

The chloride (1 eq), boronic acid (1.3 eq), K₂CO₃ (1.6 eq), and Pd-PEPPSI-Ipent (4 mol %) and EtOH (1 mL) were added to a microwave vial. The mixture was heated at 100° C. in a microwave synthesizer until the reaction was complete. Water and saturated NH₄Cl solution were added. The reaction mixture was extracted with DCM 3 times. The organic layers were combined, dried over Na₂SO₄, filtered, and the filtrate concentrated to afford the product.

### Example 2

### Preparation of (S)—N-((1H-pyrrolo[3,2-c]pyridin-2-yl)methyl)-1-chloro-3-((3,5-dimethylbenzyl)amino)-4-oxo-4,6,7,8-tetrahydropyrrolo[1,2-a]pyrazine-6-carboxamide

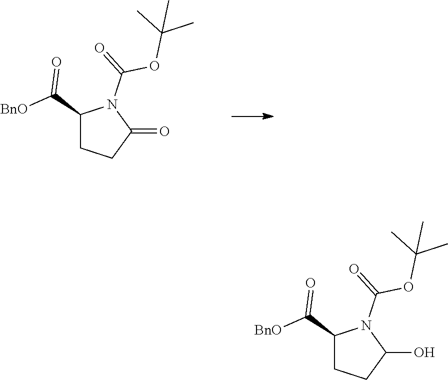 [SMILES: CC(C)(C)OC(=O)N1C(=O)CC[C@H]1C(=O)OCc1ccccc1 ; CC(C)(C)OC(=O)N1C(O)CC[C@H]1C(=O)OCc1ccccc1]

Step 1: To a solution of 2-benzyl 1-(tert-butyl) (S)-5-oxopyrrolidine-1,2-dicarboxylate (20.45 g, 64.03 mmol) in THF (100 mL) at −78° C. was added Super-Hydride® (76.8 mL, 76.84 mmol, 1.0 M in THF) with stirring for 1 h. The reaction was quenched with sat. aq. NaHCO₃ (50 mL) followed by the slow addition of H₂O₂ (10 mL, 30% in H₂O) with stirring for 30 min while warming to room temp. The volatiles were removed by evaporation followed by extraction with CH₂Cl₂ (2×100 mL). The combined organic layers were dried over Na₂SO₄, filtered, and evaporated to afford 2-benzyl 1-(tert-butyl) (2S)-5-hydroxypyrrolidine-1,2-dicarboxylate (20.2 g, 98% yield), which was used in the next step without purification.

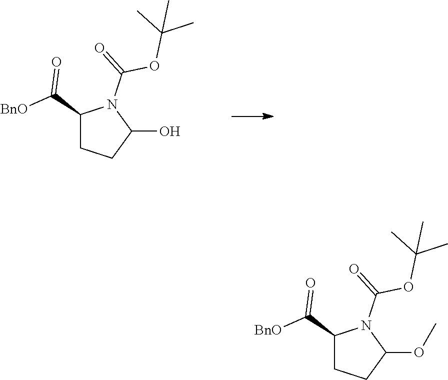 [SMILES: CC(C)(C)OC(=O)N1C(O)CC[C@H]1C(=O)OCc1ccccc1 ; COC1CC[C@@H](C(=O)OCc2ccccc2)N1C(=O)OC(C)(C)C]

Step 2: To a solution of 2-benzyl 1-(tert-butyl) (2S)-5-hydroxypyrrolidine-1,2-dicarboxylate (20.2 g, 62.9 mmol) in anhyd MeOH (100 mL) was added p-TsOH (2.4 g, 12.6 mmol). The solution was stirred at 50° C. for 3 h and evaporated to dryness. The residue was partitioned between sat. aq. NaHCO₃ (100 mL) and CH₂Cl₂ (2×100 mL). The organic layers were combined, dried over Na₂SO₄ and chromatographed (25% EtOAc-hexanes) to afford 2-benzyl 1-(tert-butyl) (2S)-5-methoxypyrrolidine-1,2-dicarboxylate (16.3 g, 77% yield) as a clear oil

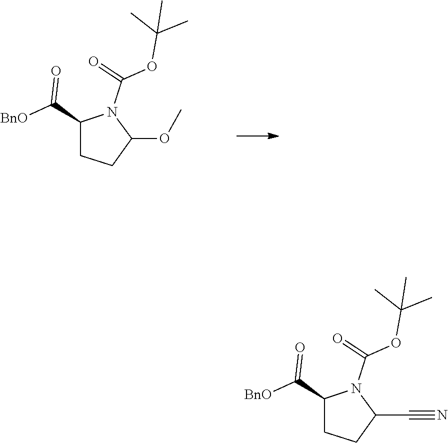 [SMILES: COC1CC[C@@H](C(=O)OCc2ccccc2)N1C(=O)OC(C)(C)C ; CC(C)(C)OC(=O)N1C(C#N)CC[C@H]1C(=O)OCc1ccccc1]

Step 3: To a solution of 2-benzyl 1-(tert-butyl) (2S)-5-methoxypyrrolidine-1,2-dicarboxylate (16.3 g, 48.6 mmol) in anhyd CH₂Cl₂ (100 mL) at −78° C. was added TMSCN (10.1 g, 102 mmol) followed by BF₃·OEt₂ (13.8 g, 97.2 mmol). The solution was stirred for 2 h and quenched with sat. aq. NaHCO₃ (100 mL). The aqueous phase was extracted with additional CH₂Cl₂ (2×100 mL) and the combined organic layers were dried over Na₂SO₄, filtered, and the filtrate evaporated to dryness. The product mixture was purified using column chromatography (25% EtOAc-hexanes) to afford two diastereomers (less polar diastereomer; 4.1 g) and (more polar diastereomer; 8.02 g) of 2-benzyl 1-(tert-butyl) (2S)-5-cyanopyrrolidine-1,2-dicarboxylate (76% yield of the combined diastereomers).

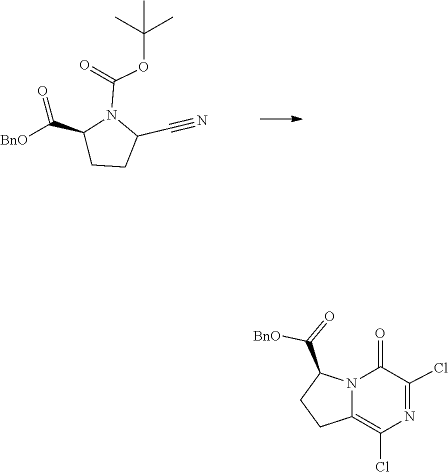 [SMILES: CC(C)(C)OC(=O)N1C(C#N)CC[C@H]1C(=O)OCc1ccccc1 ; O=C(OCc1ccccc1)[C@@H]1CCc2c(Cl)nc(Cl)c(=O)n21]

Step 4: A solution of DME (250 mL) was cooled in an ice bath with stirring for 15 min and then saturated with HCl gas by bubbling directly into the solution for approximately 4-5 min. Oxalyl chloride (30.8 g, 24.3 mmol) was added, followed by the more polar diastereomer of 2-benzyl 1-(tert-butyl) (2S)-5-cyanopyrrolidine-1,2-dicarboxylate (8.02 g, 24.2 mmol). The reaction was put under a drying tube and the solution was stirred at room temp for 60 h. Evaporation to dryness followed by chromatography (30% EtOAc-hexanes) gave benzyl (S)-1,3-dichloro-4-oxo-4,6,7,8-tetrahydropyrrolo[1,2-a]pyrazine-6-carboxylate (5.03 g, 61% yield) as an off-white solid.

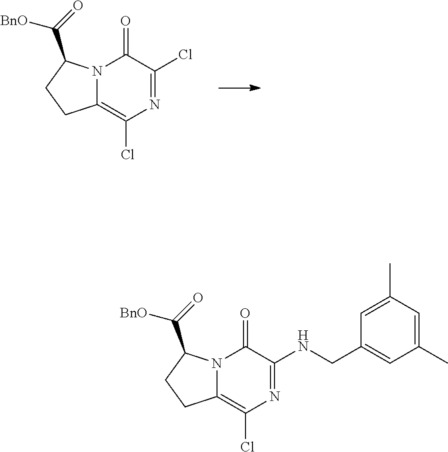 [SMILES: O=C(OCc1ccccc1)[C@@H]1CCc2c(Cl)nc(Cl)c(=O)n21 ; Cc1cc(C)cc(CNc2nc(Cl)c3n(c2=O)[C@H](C(=O)OCc2ccccc2)CC3)c1]

Step 5: General Chloro Displacement Procedure A was used to afford benzyl (S)-1-chloro-3-((3,5-dimethylbenzyl)amino)-4-oxo-4,6,7,8-tetrahydropyrrolo[1,2-a]pyrazine-6-carboxylate 226 mg, 44% yield) as a yellow solid.

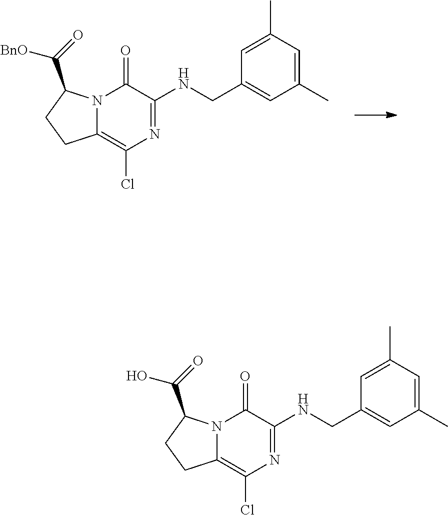 [SMILES: Cc1cc(C)cc(CNc2nc(Cl)c3n(c2=O)[C@H](C(=O)OCc2ccccc2)CC3)c1 ; Cc1cc(C)cc(CNc2nc(Cl)c3n(c2=O)[C@H](C(=O)O)CC3)c1]

Step 6: General Hydrogenolysis Procedure A was used to afford (S)-1-chloro-3-((3,5-dimethylbenzyl)amino)-4-oxo-4,6,7,8-tetrahydropyrrolo[1,2-a]pyrazine-6-carboxylic acid as a clear oil (quantitative yield).

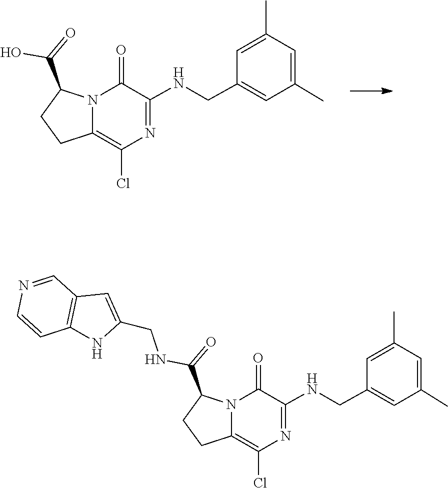 [SMILES: Cc1cc(C)cc(CNc2nc(Cl)c3n(c2=O)[C@H](C(=O)O)CC3)c1 ; Cc1cc(C)cc(CNc2nc(Cl)c3n(c2=O)[C@H](C(=O)NCc2cc4cnccc4[nH]2)CC3)c1]

Step 7: General Amide Coupling Procedure A was used to afford (S)—N-((1H-pyrrolo[3,2-c]pyridin-2-yl)methyl)-1-chloro-3-((3,5-dimethylbenzyl)amino)-4-oxo-4,6,7,8-tetrahydropyrrolo[1,2-a]pyrazine-6-carboxamide as a white solid (118 mg, 56% yield).

The following compounds were prepared according to the foregoing procedures using the appropriate amines:

| Compound Name | Cmp No. |
|---|---|
| (S)-N-((6-amino-2-methylpyridin-3-yl)methyl)-1-chloro-3-((4- | 2 |
| methoxybutyl)amino)-4-oxo-4,6,7,8-tetrahydropyrrolo[1,2-a]pyrazine-6- | |
| carboxamide | |
| (S)-N-((1H-pyrrolo[3,2-c]pyridin-2-yl)methyl)-1-chloro-3-((3- | 3 |
| ethylbenzyl)amino)-4-oxo-4,6,7,8-tetrahydropyrrolo[1,2-a]pyrazine-6- | |
| carboxamide | |
| (S)-N-((6-amino-2-methylpyridin-3-yl)methyl)-1-chloro-3-((3- | 4 |
| ethylphenyl)amino)-4-oxo-4,6,7,8-tetrahydropyrrolo[1,2-a]pyrazine-6- | |
| carboxamide | |
| (S)-N-((6-amino-2-methylpyridin-3-yl)methyl)-1-chloro-3-((3- | 5 |
| (hydroxymethyl)benzyl)amino)-4-oxo-4,6,7,8-tetrahydropyrrolo[1,2- | |
| a]pyrazine-6-carboxamide | |
| (S)-N-((1H-pyrrolo[3,2-c]pyridin-2-yl)methyl)-1-chloro-3-((3- | 6 |
| (hydroxymethyl)benzyl)amino)-4-oxo-4,6,7,8-tetrahydropyrrolo[1,2- | |
| a]pyrazine-6-carboxamide | |
| (S)-N-((1H-pyrrolo[3,2-c]pyridin-2-yl)methyl)-1-chloro-3-((3-methoxy-5- | 7 |
| methylbenzyl)amino)-4-oxo-4,6,7,8-tetrahydropyrrolo[1,2-a]pyrazine-6- | |
| carboxamide | |
| (S)-N-((6-amino-2-methylpyridin-3-yl)methyl)-1-chloro-3-((3-methoxy-5- | 8 |
| methylbenzyl)amino)-4-oxo-4,6,7,8-tetrahydropyrrolo[1,2-a]pyrazine-6- | |
| carboxamide | |
| (S)-N-((1H-pyrrolo[3,2-c]pyridin-2-yl)methyl)-1-chloro-3-((2- | 9 |
| methylbenzyl)amino)-4-oxo-4,6,7,8-tetrahydropyrrolo[1,2-a]pyrazine-6- | |
| carboxamide | |
| (S)-N-((6-amino-2-methylpyridin-3-yl)methyl)-1-chloro-3-((2,5- | 10 |
| dimethylbenzyl)amino)-4-oxo-4,6,7,8-tetrahydropyrrolo[1,2-a]pyrazine-6- | |
| carboxamide | |
| (S)-N-((1H-pyrrolo[3,2-c]pyridin-2-yl)methyl)-1-chloro-3-((2,5- | 11 |
| dimethylbenzyl)amino)-4-oxo-4,6,7,8-tetrahydropyrrolo[1,2-a]pyrazine-6- | |
| carboxamide | |
| (S)-N-((6-amino-2-methylpyridin-3-yl)methyl)-1-chloro-3-((2-fluoro-3- | 12 |
| methylbenzyl)amino)-4-oxo-4,6,7,8-tetrahydropyrrolo[1,2-a]pyrazine-6- | |
| carboxamide | |
| (S)-N-((1H-pyrrolo[3,2-c]pyridin-2-yl)methyl)-1-chloro-3-((2-fluoro-3- | 13 |
| methylbenzyl)amino)-4-oxo-4,6,7,8-tetrahydropyrrolo[1,2-a]pyrazine-6- | |
| carboxamide | |
| (6S,8S)-N-((1H-pyrrolo[3,2-c]pyridin-2-yl)methyl)-1-chloro-3-((3,5- | 14 |
| dimethylbenzyl)amino)-8-ethyl-4-oxo-4,6,7,8-tetrahydropyrrolo[1,2- | |
| a]pyrazine-6-carboxamide | |
| (S)-N-((1H-pyrrolo[3,2-c]pyridin-2-yl)methyl)-1-chloro-3-((2,3- | 15 |
| dimethylbenzyl)amino)-4-oxo-4,6,7,8-tetrahydropyrrolo[1,2-a]pyrazine-6- | |
| carboxamide | |
| (S)-N-((6-amino-2-methylpyridin-3-yl)methyl)-1-chloro-3-((3- | 16 |
| (difluoromethyl)benzyl)amino)-4-oxo-4,6,7,8-tetrahydropyrrolo[1,2- | |
| a]pyrazine-6-carboxamide | |
| (S)-N-((1H-pyrrolo[3,2-c]pyridin-2-yl)methyl)-1-chloro-3-((3- | 17 |
| (difluoromethyl)benzyl)amino)-4-oxo-4,6,7,8-tetrahydropyrrolo[1,2- | |
| a]pyrazine-6-carboxamide | |
| (S)-N-((6-amino-2-methylpyridin-3-yl)methyl)-1-chloro-3-((3,3- | 18 |
| difluoropropyl)amino)-4-oxo-4,6,7,8-tetrahydropyrrolo[1,2-a]pyrazine-6- | |
| carboxamide | |
| (S)-N-((1H-pyrrolo[3,2-c]pyridin-2-yl)methyl)-1-chloro-3-((3,3- | 19 |
| difluoropropyl)amino)-4-oxo-4,6,7,8-tetrahydropyrrolo[1,2-a]pyrazine-6- | |
| carboxamide | |
| (S)-N-((6-amino-2-methylpyridin-3-yl)methyl)-1-chloro-3-((2-fluoro-5- | 20 |
| methylbenzyl)amino)-4-oxo-4,6,7,8-tetrahydropyrrolo[1,2-a]pyrazine-6- | |
| carboxamide | |
| (S)-N-((1H-pyrrolo[3,2-c]pyridin-2-yl)methyl)-1-chloro-3-((2-fluoro-5- | 21 |
| methylbenzyl)amino)-4-oxo-4,6,7,8-tetrahydropyrrolo[1,2-a]pyrazine-6- | |
| carboxamide | |

The following compounds were prepared according to the foregoing procedures using the appropriate amines, except that purification of the amide coupling step consisted of a single amine column with the product eluted using a DCM-MeOH gradient:

| Compound Name | Cmp No. |
|---|---|
| (S)-N-((1H-pyrrolo[3,2-c]pyridin-2-yl)methyl)-1-chloro-3-((4- | 22 |
| methoxybutyl)amino)-4-oxo-4,6,7,8-tetrahydropyrrolo[1,2-a]pyrazine-6- | |
| carboxamide | |
| (S)-N-((1H-pyrrolo[3,2-c]pyridin-2-yl)methyl)-1-chloro-3-((3-methyl-5- | 23 |
| (trifluoromethyl)benzyl)amino)-4-oxo-4,6,7,8-tetrahydropyrrolo[1,2- | |
| a]pyrazine-6-carboxamide | |

The following compound was prepared according to the foregoing procedures using the appropriate amine, except that purification of the amide coupling step consisted of prep HPLC followed by silica gel column chromatography with the product eluted using a DCM-MeOH gradient:

| | Cmp |
|---|---|
| Compound Name | No. |
| (S)-N-((6-amino-2-methylpyridin-3-yl)methyl)-1-chloro-3-((3,5- | 24 |
| dimethylbenzyl)amino)-4-oxo-4,6,7,8-tetrahydropyrrolo[1,2-a]pyrazine-6- | |
| carboxamide trifluoroacetate | |

The following compounds were prepared according to the foregoing procedures using the appropriate amines, except that purification of the amide coupling step consisted of two amine columns with the product eluted using a DCM-MeOH gradient:

| | Cmp |
|---|---|
| Compound Name | No. |
| (S)-N-((1H-pyrrolo[3,2-c]pyridin-2-yl)methyl)-1-chloro-3-((3- | 25 |
| fluorobenzyl)amino)-4-oxo-4,6,7,8-tetrahydropyrrolo[1,2-a]pyrazine-6- | |
| carboxamide | |
| (S)-N-((1H-pyrrolo[3,2-c]pyridin-2-yl)methyl)-1-chloro-3-((3,4- | 26 |
| dimethylbenzyl)amino)-4-oxo-4,6,7,8-tetrahydropyrrolo[1,2-a]pyrazine-6- | |
| carboxamide | |

The following compounds were prepared according to the foregoing procedures using the appropriate amines, except that purification of the amide coupling step consisted of an amine column with the product eluted using a DCM-MeOH gradient followed by prep HPLC:

| | Cmp |
|---|---|
| Compound Name | No. |
| (S)-N-((6-amino-2-methylpyridin-3-yl)methyl)-1-chloro-3-((3,4- | 27 |
| dimethylbenzyl)amino)-4-oxo-4,6,7,8-tetrahydropyrrolo[1,2-a]pyrazine-6- | |
| carboxamide trifluoroacetate | |
| (S)-N-((6-amino-2-methylpyridin-3-yl)methyl)-1-chloro-3-((2- | 28 |
| methylbenzyl)amino)-4-oxo-4,6,7,8-tetrahydropyrrolo[1,2-a]pyrazine-6- | |
| carboxamide trifluoroacetate | |

The following compound was prepared according to the foregoing procedures using the appropriate amine, except that purification of the amide coupling step consisted of a silica gel column with the product eluted using a heptane-EtOAc gradient followed by prep HPLC:

| | Cmp |
|---|---|
| Compound Name | No. |
| (S)-N-((2-amino-1H-benzo[d]imidazol-5-yl)methyl)-3-(benzylamino)-1- | 29 |
| chloro-4-oxo-4,6,7,8-tetrahydropyrrolo[1,2-a]pyrazine-6-carboxamide | |
| trifluoroacetate | |

The following compound was prepared according to the foregoing procedures using the appropriate amine, except that MeOH was used as a solvent during the hydrogenolysis step, and purification of the amide coupling step consisted of prep HPLC after column chromatography:

| | Cmp |
|---|---|
| Compound Name | No. |
| (S)-N-((6-amino-2-methylpyridin-3-yl)methyl)-1-chloro-3-((4- | 30 |
| chlorobenzyl)amino)-4-oxo-4,6,7,8-tetrahydropyrrolo[1,2-a]pyrazine-6- | |
| carboxamide trifluoroacetate | |

### Example 3

### Preparation of (S)-1-chloro-N-((5,6-dihydro-4H-thieno[2,3-c]pyrrol-2-yl)methyl)-3-((3,5-dimethylbenzyl)amino)-4-oxo-4,6,7,8-tetrahydropyrrolo[1,2-a]pyrazine-6-carboxamide

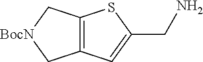 [SMILES: CC(C)(C)OC(=O)N1Cc2cc(CN)sc2C1]

Step 1: The above compound was synthesized according to the procedures outlined in patent WO2021113682 A1.

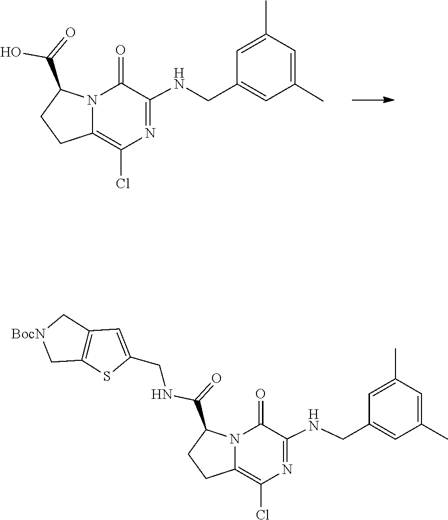 [SMILES: Cc1cc(C)cc(CNc2nc(Cl)c3n(c2=O)[C@H](C(=O)O)CC3)c1 ; Cc1cc(C)cc(CNc2nc(Cl)c3n(c2=O)[C@H](C(=O)NCc2cc4c(s2)CN(C(=O)OC(C)(C)C)C4)CC3)c1]

Step 2: The above compound was synthesized according to General Amide Coupling Procedure A, except that only one amine column was performed during purification, to afford benzyl (S)—N-((1H-pyrrolo[3,2-c]pyridin-2-yl)methyl)-1-chloro-3-((3,5-dimethylbenzyl)amino)-4-oxo-4,6,7,8-tetrahydropyrrolo[1,2-a]pyrazine-6-carboxamide as a white solid (242 mg, 96% yield).

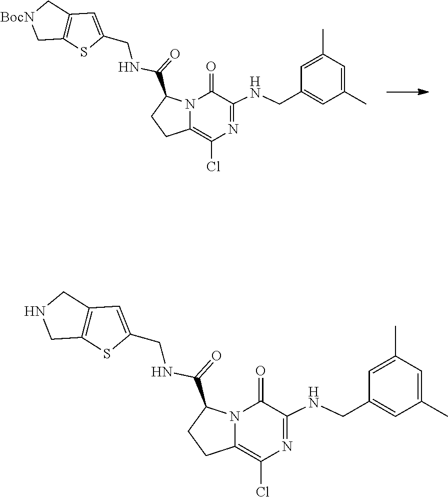 [SMILES: Cc1cc(C)cc(CNc2nc(Cl)c3n(c2=O)[C@H](C(=O)NCc2cc4c(s2)CN(C(=O)OC(C)(C)C)C4)CC3)c1 ; Cc1cc(C)cc(CNc2nc(Cl)c3n(c2=O)[C@H](C(=O)NCc2cc4c(s2)CNC4)CC3)c1]

Step 3: The above compound was synthesized according to General Boc Removal Procedure A, except that prep HPLC purification was performed before the amine column, to afford (S)-1-chloro-N-((5,6-dihydro-4H-thieno[2,3-c]pyrrol-2-yl)methyl)-3-((3,5-dimethylbenzyl)amino)-4-oxo-4,6,7,8-tetrahydropyrrolo[1,2-a]pyrazine-6-carboxamide (60.4 mg, 28% yield).

The following compounds were prepared according to the foregoing procedures using the appropriate amines, except that purification of the amide coupling step consisted of a single amine column with the product eluted using a DCM-MeOH gradient:

| | Cmp |
|---|---|
| Compound Name | No. |
| (S)-1-chloro-N-((5,6-dihydro-4H-thieno[2,3-c]pyrrol-2-yl)methyl)-3-((2- | 32 |
| fluoro-3-methylbenzyl)amino)-4-oxo-4,6,7,8-tetrahydropyrrolo[1,2- | |
| a]pyrazine-6-carboxamide | |
| (S)-1-chloro-N-((5,6-dihydro-4H-thieno[2,3-c]pyrrol-2-yl)methyl)-3-((2,3- | 33 |
| dimethylbenzyl)amino)-4-oxo-4,6,7,8-tetrahydropyrrolo[1,2-a]pyrazine-6- | |
| carboxamide | |
| (S)-1-chloro-3-((3-(difluoromethyl)benzyl)amino)-N-((5,6-dihydro-4H- | 34 |
| thieno[2,3-c]pyrrol-2-yl)methyl)-4-oxo-4,6,7,8-tetrahydropyrrolo[1,2- | |
| a]pyrazine-6-carboxamide | |

The following compound was prepared according to the foregoing procedures using the appropriate amine, except that the crude material from the amide coupling step was moved to the Boc removal step without purification:

| | Cmp |
|---|---|
| Compound Name | No. |
| (S)-3-(benzylamino)-1-chloro-N-((5,6-dihydro-4H-thieno[2,3-c]pyrrol-2- | 35 |
| yl)methyl)-4-oxo-4,6,7,8-tetrahydropyrrolo[1,2-a]pyrazine-6-carboxamide | |

### Example 4

### Preparation of (S)—N-((6-amino-2-methylpyridin-3-yl)methyl)-3-((3,5-dimethylbenzyl)amino)-4-oxo-4,6,7,8-tetrahydropyrrolo[1,2-a]pyrazine-6-carboxamide

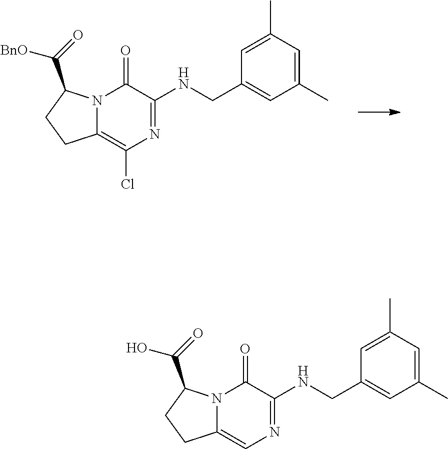 [SMILES: Cc1cc(C)cc(CNc2nc(Cl)c3n(c2=O)[C@H](C(=O)OCc2ccccc2)CC3)c1 ; Cc1cc(C)cc(CNc2ncc3n(c2=O)[C@H](C(=O)O)CC3)c1]

Step 1: The above compound was synthesized according to General Hydrogenolysis Procedure B to afford (S)-3-((3,5-dimethylbenzyl)amino)-4-oxo-4,6,7,8-tetrahydropyrrolo[1,2-a]pyrazine-6-carboxylic acid as a clear oil (quantitative yield).

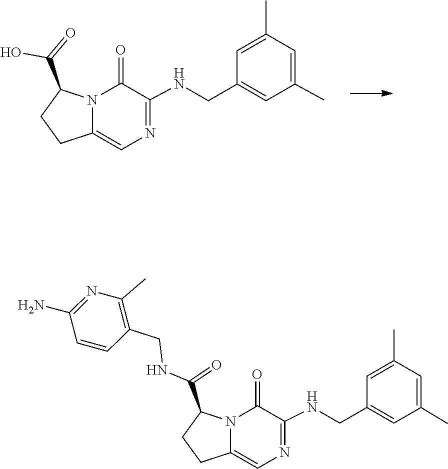 [SMILES: Cc1cc(C)cc(CNc2ncc3n(c2=O)[C@H](C(=O)O)CC3)c1 ; Cc1cc(C)cc(CNc2ncc3n(c2=O)[C@H](C(=O)NCc2ccc(N)nc2C)CC3)c1]

Step 2: The above compound was synthesized according to General Amide Coupling Procedure A, except that an additional amine column was performed, to afford (S)—N-((6-amino-2-methylpyridin-3-yl)methyl)-3-((3,5-dimethylbenzyl)amino)-4-oxo-4,6,7,8-tetrahydropyrrolo[1,2-a]pyrazine-6-carboxamide as a white solid (76.5 mg, 60% yield).

The following compounds were prepared according to the foregoing procedures with the appropriate amines:

| | Cmp |
|---|---|
| Compound Name | No. |
| (S)-N-((6-amino-2-methylpyridin-3-yl)methyl)-3-((3-methyl-5- | 37 |
| (trifluoromethyl)benzyl)amino)-4-oxo-4,6,7,8-tetrahydropyrrolo[1,2- | |
| a]pyrazine-6-carboxamide | |
| (S)-N-((6-amino-2-methylpyridin-3-yl)methyl)-3-((3,4- | 38 |
| dimethylbenzyl)amino)-4-oxo-4,6,7,8-tetrahydropyrrolo[1,2-a]pyrazine-6- | |
| carboxamide | |

The following compounds were prepared according to the foregoing procedures with the appropriate amines, except that prep HPLC purification was used during the amine column in the final amide coupling step:

| | Cmp |
|---|---|
| Compound Name | No. |
| (S)-N-((1H-pyrrolo[3,2-c]pyridin-2-yl)methyl)-3-((3- | 39 |
| methoxybenzyl)amino)-4-oxo-4,6,7,8-tetrahydropyrrolo[1,2-a]pyrazine-6- | |
| carboxamide trifluoroacetate | |
| (S)-3-((3,5-dimethylbenzyl)amino)-4-oxo-N-(pyridin-2-ylmethyl)-4,6,7,8- | 40 |
| tetrahydropyrrolo[1,2-a]pyrazine-6-carboxamide trifluoroacetate | |
| (S)-3-((3,5-dimethylbenzyl)amino)-N-((1-methyl-1H-pyrrolo[3,2-c]pyridin- | 41 |
| 2-yl)methyl)-4-oxo-4,6,7,8-tetrahydropyrrolo[1,2-a]pyrazine-6- | |
| carboxamide trifluoroacetate | |
| (S)-3-((3,5-dimethylbenzyl)amino)-N-((5-methyl-5,6-dihydro-4H- | 42 |
| thieno[2,3-c]pyrrol-2-yl)methyl)-4-oxo-4,6,7,8-tetrahydropyrrolo[1,2- | |
| a]pyrazine-6-carboxamide trifluoroacetate | |
| (6S)-N-(1-(1H-pyrrolo[3,2-c]pyridin-2-yl)ethyl)-3-((3,5- | 43 |
| dimethylbenzyl)amino)-4-oxo-4,6,7,8-tetrahydropyrrolo[1,2-a]pyrazine-6- | |
| carboxamide trifluoroacetate | |

The following compounds were prepared according to the foregoing procedures with the appropriate amines, except that purification of the amide coupling step consisted of only an amine column, and the product was eluted using a DCM-MeOH gradient:

| | Cmp |
|---|---|
| Compound Name | No. |
| (S)-N-((1H-pyrrolo[3,2-c]pyridin-2-yl)methyl)-3-((3,4- | 44 |
| dimethylbenzyl)amino)-4-oxo-4,6,7,8-tetrahydropyrrolo[1,2-a]pyrazine-6- | |
| carboxamide | |
| (S)-N-((1H-pyrrolo[3,2-c]pyridin-2-yl)methyl)-3-((3-ethylbenzyl)amino)-4- | 45 |
| oxo-4,6,7,8-tetrahydropyrrolo[1,2-a]pyrazine-6-carboxamide | |

The following compounds were prepared according to the foregoing procedures with the appropriate amines, except that purification of the amide coupling step consisted of an amine column, and the product was eluted using a DCM-MeOH gradient followed by a silica gel column with the product eluted using a DCM-MeOH gradient:

| | Cmp |
|---|---|
| Compound Name | No. |
| (S)-N-((6-amino-2-methylpyridin-3-yl)methyl)-3-((3-ethylbenzyl)amino)-4- | 46 |
| oxo-4,6,7,8-tetrahydropyrrolo[1,2-a]pyrazine-6-carboxamide | |
| (S)-N-((6-amino-2-methylpyridin-3-yl)methyl)-3-((3- | 47 |
| (hydroxymethyl)benzyl)amino)-4-oxo-4,6,7,8-tetrahydropyrrolo[1,2- | |
| a]pyrazine-6-carboxamide | |
| (S)-N-((1H-pyrrolo[3,2-c]pyridin-2-yl)methyl)-3-((3-methoxy-5- | 48 |
| methylbenzyl)amino)-4-oxo-4,6,7,8-tetrahydropyrrolo[1,2-a]pyrazine-6- | |
| carboxamide | |
| (S)-N-((6-amino-2-methylpyridin-3-yl)methyl)-3-((3-methoxy-5- | 49 |
| methylbenzyl)amino)-4-oxo-4,6,7,8-tetrahydropyrrolo[1,2-a]pyrazine-6- | |
| carboxamide | |
| (S)-3-((3,5-dimethylbenzyl)amino)-N-((6-methyl-1H-pyrrolo[3,2-c]pyridin- | 50 |
| 2-yl)methyl)-4-oxo-4,6,7,8-tetrahydropyrrolo[1,2-a]pyrazine-6- | |
| carboxamide | |
| (S)-3-((3-methoxy-5-methylbenzyl)amino)-N-((6-methyl-1H-pyrrolo[3,2- | 51 |
| c]pyridin-2-yl)methyl)-4-oxo-4,6,7,8-tetrahydropyrrolo[1,2-a]pyrazine-6- | |
| carboxamide | |
| (S)-N-((6-amino-2-methylpyridin-3-yl)methyl)-3-((2-methylbenzyl)amino)- | 52 |
| 4-oxo-4,6,7,8-tetrahydropyrrolo[1,2-a]pyrazine-6-carboxamide | |
| (S)-N-((1H-pyrrolo[3,2-c]pyridin-2-yl)methyl)-3-((2-methylbenzyl)amino)- | 53 |
| 4-oxo-4,6,7,8-tetrahydropyrrolo[1,2-a]pyrazine-6-carboxamide | |
| (S)-N-((1H-pyrrolo[3,2-c]pyridin-2-yl)methyl)-3-((3,5- | 54 |
| difluorobenzyl)amino)-4-oxo-4,6,7,8-tetrahydropyrrolo[1,2-a]pyrazine-6- | |
| carboxamide | |
| (S)-N-((6-amino-2-methylpyridin-3-yl)methyl)-3-((2,5- | 55 |
| dimethylbenzyl)amino)-4-oxo-4,6,7,8-tetrahydropyrrolo[1,2-a]pyrazine-6- | |
| carboxamide | |
| (S)-N-((1H-pyrrolo[3,2-c]pyridin-2-yl)methyl)-3-((2,5- | 56 |
| dimethylbenzyl)amino)-4-oxo-4,6,7,8-tetrahydropyrrolo[1,2-a]pyrazine-6- | |
| carboxamide | |
| (S)-3-((2,5-dimethylbenzyl)amino)-N-((6-methyl-1H-pyrrolo[3,2-c]pyridin- | 57 |
| 2-yl)methyl)-4-oxo-4,6,7,8-tetrahydropyrrolo[1,2-a]pyrazine-6- | |
| carboxamide | |
| (S)-3-((2,3-dimethylbenzyl)amino)-N-((6-methyl-1H-pyrrolo[3,2-c]pyridin- | 58 |
| 2-yl)methyl)-4-oxo-4,6,7,8-tetrahydropyrrolo[1,2-a]pyrazine-6- | |
| carboxamide | |
| (S)-N-((1H-pyrrolo[3,2-c]pyridin-2-yl)methyl)-3-((2,3- | 59 |
| dimethylbenzyl)amino)-4-oxo-4,6,7,8-tetrahydropyrrolo[1,2-a]pyrazine-6- | |
| carboxamide | |
| (S)-N-((6-amino-2-methylpyridin-3-yl)methyl)-3-((2,3- | 60 |
| dimethylbenzyl)amino)-4-oxo-4,6,7,8-tetrahydropyrrolo[1,2-a]pyrazine-6- | |
| carboxamide | |
| (S)-3-((2-fluoro-3-methylbenzyl)amino)-N-((6-methyl-1H-pyrrolo[3,2- | 61 |
| c]pyridin-2-yl)methyl)-4-oxo-4,6,7,8-tetrahydropyrrolo[1,2-a]pyrazine-6- | |
| carboxamide | |
| (S)-N-((6-amino-2-methylpyridin-3-yl)methyl)-3-((2-fluoro-3- | 62 |
| methylbenzyl)amino)-4-oxo-4,6,7,8-tetrahydropyrrolo[1,2-a]pyrazine-6- | |
| carboxamide | |
| (S)-N-((1H-pyrrolo[3,2-c]pyridin-2-yl)methyl)-3-((5-fluoro-2- | 63 |
| methylbenzyl)amino)-4-oxo-4,6,7,8-tetrahydropyrrolo[1,2-a]pyrazine-6- | |
| carboxamide | |
| (S)-3-((5-fluoro-2-methylbenzyl)amino)-N-((6-methyl-1H-pyrrolo[3,2- | 64 |
| c]pyridin-2-yl)methyl)-4-oxo-4,6,7,8-tetrahydropyrrolo[1,2-a]pyrazine-6- | |
| carboxamide | |
| (S)-N-((6-amino-2-methylpyridin-3-yl)methyl)-3-((5-fluoro-2- | 65 |
| methylbenzyl)amino)-4-oxo-4,6,7,8-tetrahydropyrrolo[1,2-a]pyrazine-6- | |
| carboxamide | |
| (S)-N-((6-amino-2-methylpyridin-3-yl)methyl)-3-((3-(2-hydroxypropan-2- | 66 |
| yl)benzyl)amino)-4-oxo-4,6,7,8-tetrahydropyrrolo[1,2-a]pyrazine-6- | |
| carboxamide | |
| (S)-N-((1H-pyrrolo[3,2-c]pyridin-2-yl)methyl)-3-((3-(2-hydroxypropan-2- | 67 |
| yl)benzyl)amino)-4-oxo-4,6,7,8-tetrahydropyrrolo[1,2-a]pyrazine-6- | |
| carboxamide | |
| (S)-3-((3-(2-hydroxypropan-2-yl)benzyl)amino)-N-((6-methyl-1H- | 68 |
| pyrrolo[3,2-c]pyridin-2-yl)methyl)-4-oxo-4,6,7,8-tetrahydropyrrolo[1,2- | |
| a]pyrazine-6-carboxamide | |
| (S)-N-((6-amino-2-methylpyridin-3-yl)methyl)-3-((3-fluoro-5- | 69 |
| methylbenzyl)amino)-4-oxo-4,6,7,8-tetrahydropyrrolo[1,2-a]pyrazine-6- | |
| carboxamide | |
| (S)-N-((1H-pyrrolo[3,2-c]pyridin-2-yl)methyl)-3-((3-fluoro-5- | 70 |
| methylbenzyl)amino)-4-oxo-4,6,7,8-tetrahydropyrrolo[1,2-a]pyrazine-6- | |
| carboxamide | |
| (S)-3-((3-fluoro-5-methylbenzyl)amino)-N-((6-methyl-1H-pyrrolo[3,2- | 71 |
| c]pyridin-2-yl)methyl)-4-oxo-4,6,7,8-tetrahydropyrrolo[1,2-a]pyrazine-6- | |
| carboxamide | |
| (S)-N-((6-aminopyridin-3-yl)methyl)-3-((3-fluoro-5-methylbenzyl)amino)- | 72 |
| 4-oxo-4,6,7,8-tetrahydropyrrolo[1,2-a]pyrazine-6-carboxamide | |
| (S)-N-((1H-pyrrolo[3,2-c]pyridin-2-yl)methyl)-4-oxo-3-(((R)-1-(m- | 73 |
| tolyl)ethyl)amino)-4,6,7,8-tetrahydropyrrolo[1,2-a]pyrazine-6-carboxamide | |
| (S)-N-((1H-pyrrolo[3,2-c]pyridin-2-yl)methyl)-4-oxo-3-(((S)-1-(m- | 74 |
| tolyl)ethyl)amino)-4,6,7,8-tetrahydropyrrolo[1,2-a]pyrazine-6-carboxamide | |
| (S)-N-((1H-pyrrolo[3,2-c]pyridin-2-yl)methyl)-3-(((S)-1-(3,5- | 75 |
| dimethylphenyl)-2-hydroxyethyl)amino)-4-oxo-4,6,7,8- | |
| tetrahydropyrrolo[1,2-a]pyrazine-6-carboxamide | |
| (S)-N-((6-amino-2-methylpyridin-3-yl)methyl)-3-(((S)-1-(3,5- | 76 |
| dimethylphenyl)-2-hydroxyethyl)amino)-4-oxo-4,6,7,8- | |
| tetrahydropyrrolo[1,2-a]pyrazine-6-carboxamide | |
| (S)-N-((6-amino-2-methylpyridin-3-yl)methyl)-3-((2-fluoro-5- | 77 |
| methylbenzyl)amino)-4-oxo-4,6,7,8-tetrahydropyrrolo[1,2-a]pyrazine-6- | |
| carboxamide | |
| (S)-N-((1H-pyrrolo[3,2-c]pyridin-2-yl)methyl)-3-((2-fluoro-5- | 78 |
| methylbenzyl)amino)-4-oxo-4,6,7,8-tetrahydropyrrolo[1,2-a]pyrazine-6- | |
| carboxamide | |
| (S)-N-((6-aminopyridin-3-yl)methyl)-3-((2-fluoro-5-methylbenzyl)amino)- | 79 |
| 4-oxo-4,6,7,8-tetrahydropyrrolo[1,2-a]pyrazine-6-carboxamide | |
| (S)-3-((2-fluoro-5-methylbenzyl)amino)-N-((6-methyl-1H-pyrrolo[3,2- | 80 |
| c]pyridin-2-yl)methyl)-4-oxo-4,6,7,8-tetrahydropyrrolo[1,2-a]pyrazine-6- | |
| carboxamide | |
| (S)-N-((1H-pyrrolo[3,2-c]pyridin-2-yl)methyl)-4-oxo-3-(((R)-1-(0- | 81 |
| tolyl)ethyl)amino)-4,6,7,8-tetrahydropyrrolo[1,2-a]pyrazine-6-carboxamide | |
| (S)-N-((6-amino-2-methylpyridin-3-yl)methyl)-4-oxo-3-(((R)-1-(o- | 82 |
| tolyl)ethyl)amino)-4,6,7,8-tetrahydropyrrolo[1,2-a]pyrazine-6-carboxamide | |
| (S)-N-((1H-pyrrolo[3,2-c]pyridin-2-yl)methyl)-4-oxo-3-(((S)-1-(o- | 83 |
| tolyl)ethyl)amino)-4,6,7,8-tetrahydropyrrolo[1,2-a]pyrazine-6-carboxamide | |
| (S)-N-((1H-pyrrolo[3,2-c]pyridin-2-yl)methyl)-4-oxo-3-(3-phenylazetidin- | 84 |
| 1-yl)-4,6,7,8-tetrahydropyrrolo[1,2-a]pyrazine-6-carboxamide | |

The following compounds were prepared according to the foregoing procedures with the appropriate amines, except that purification of the amide coupling step consisted of only a silica gel column with the product eluted using a DCM-MeOH gradient:

| | Cmp |
|---|---|
| Compound Name | No. |
| (S)-3-((3,5-dimethylbenzyl)amino)-4-oxo-N-(pyridin-3-ylmethyl)-4,6,7,8- | 85 |
| tetrahydropyrrolo[1,2-a]pyrazine-6-carboxamide | |
| (S)-3-((3,5-dimethylbenzyl)amino)-4-oxo-N-(pyridin-4-ylmethyl)-4,6,7,8- | 86 |
| tetrahydropyrrolo[1,2-a]pyrazine-6-carboxamide | |
| (S)-N-((6-aminopyridin-3-yl)methyl)-3-((3,5-dimethylbenzyl)amino)-4- | 87 |
| oxo-4,6,7,8-tetrahydropyrrolo[1,2-a]pyrazine-6-carboxamide | |
| (S)-N-((2-aminopyridin-4-yl)methyl)-3-((3,5-dimethylbenzyl)amino)-4- | 88 |
| oxo-4,6,7,8-tetrahydropyrrolo[1,2-a]pyrazine-6-carboxamide | |

The following compound was prepared according to the foregoing procedures with the appropriate amine, except that purification of the amide coupling step consisted of only prep HPLC:

| | Cmp |
|---|---|
| Compound Name | No. |
| (S)-1-chloro-3-((3,5-dimethylbenzyl)amino)-N-((2-methylpyridin-3- | 89 |
| yl)methyl)-4-oxo-4,6,7,8-tetrahydropyrrolo[1,2-a]pyrazine-6-carboxamide | |
| trifluoroacetate | |

### Example 5

### Preparation of ((S)—N-((5,6-dihydro-4H-thieno[2,3-c]pyrrol-2-yl)methyl)-4-oxo-3-(((R)-1-(m-tolyl)ethyl)amino)-4,6,7,8-tetrahydropyrrolo[1,2-a]pyrazine-6-carboxamide

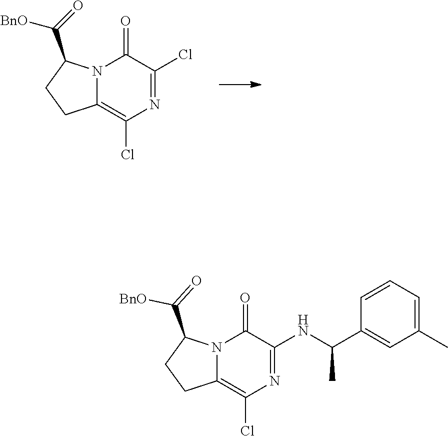 [SMILES: O=C(OCc1ccccc1)[C@@H]1CCc2c(Cl)nc(Cl)c(=O)n21 ; Cc1cccc([C@@H](C)Nc2nc(Cl)c3n(c2=O)[C@H](C(=O)OCc2ccccc2)CC3)c1]

Step 1: The above compound was synthesized according to General Chloro Displacement Procedure A to afford benzyl (S)-1-chloro-4-oxo-3-(((R)-1-(in-tolyl)ethyl)amino)-4,6,7,8-tetrahydropyrrolo[1,2-a]pyrazine-6-carboxylate (92 mg, 24% yield) as a white solid.

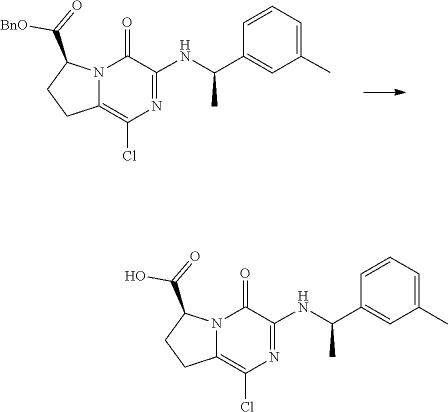 [SMILES: Cc1cccc([C@@H](C)Nc2nc(Cl)c3n(c2=O)[C@H](C(=O)OCc2ccccc2)CC3)c1 ; Cc1cccc([C@@H](C)Nc2nc(Cl)c3n(c2=O)[C@H](C(=O)O)CC3)c1]

Step 2: The above compound was synthesized according to General Hydrogenolysis Procedure A to afford (S)-1-chloro-4-oxo-3-(((R)-1-(m-tolyl)ethyl)amino)-4,6,7,8-tetrahydropyrrolo[1,2-a]pyrazine-6-carboxylic acid (quantitative yield).

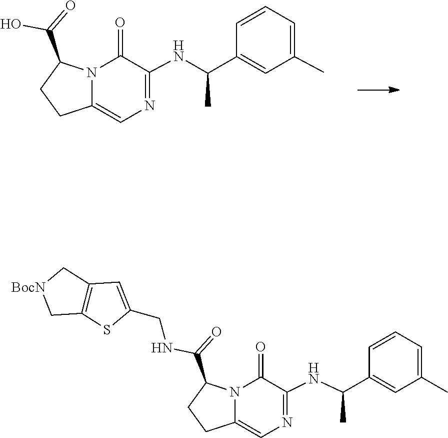 [SMILES: Cc1cccc([C@@H](C)Nc2ncc3n(c2=O)[C@H](C(=O)O)CC3)c1 ; Cc1cccc([C@@H](C)Nc2ncc3n(c2=O)[C@H](C(=O)NCc2cc4c(s2)CN(C(=O)OC(C)(C)C)C4)CC3)c1]

Step 3: The above compound was synthesized according to General Amide Coupling Procedure A with the appropriate amine to afford tert-butyl 2-(((S)-4-oxo-3-(((R)-1-(m-tolyl)ethyl)amino)-4,6,7,8-tetrahydropyrrolo[1,2-a]pyrazine-6-carboxamido)methyl)-4,6-dihydro-5H-thieno[2,3-c]pyrrole-5-carboxylate as a white solid (42 mg, 73% yield).

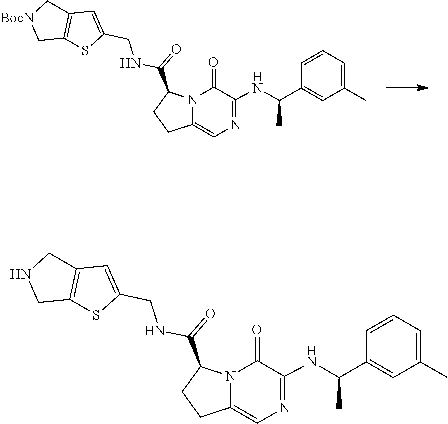 [SMILES: Cc1cccc([C@@H](C)Nc2ncc3n(c2=O)[C@H](C(=O)NCc2cc4c(s2)CN(C(=O)OC(C)(C)C)C4)CC3)c1 ; Cc1cccc([C@@H](C)Nc2ncc3n(c2=O)[C@H](C(=O)NCc2cc4c(s2)CNC4)CC3)c1]

Step 4: The above compound was synthesized according to General Boc Removal Procedure A to afford (S)—N-((5,6-dihydro-4H-thieno[2,3-c]pyrrol-2-yl)methyl)-4-oxo-3-(((R)-1-(m-tolyl)ethyl)amino)-4,6,7,8-tetrahydropyrrolo[1,2-a]pyrazine-6-carboxamide (16 mg, 65% yield).

The following compounds were prepared according to the foregoing procedures with the appropriate amines:

| | Cmp |
|---|---|
| Compound Name | No. |
| (S)-N-((5,6-dihydro-4H-thieno[2,3-c]pyrrol-2-yl)methyl)-3-((3-methoxy-5- | 91 |
| methylbenzyl)amino)-4-oxo-4,6,7,8-tetrahydropyrrolo[1,2-a]pyrazine-6- | |
| carboxamide | |
| (S)-N-((5,6-dihydro-4H-thieno[2,3-c]pyrrol-2-yl)methyl)-3-(((S)-1-(3,5- | 92 |
| dimethylphenyl)-2-hydroxyethyl)amino)-4-oxo-4,6,7,8- | |
| tetrahydropyrrolo[1,2-a]pyrazine-6-carboxamide | |
| (S)-N-((5,6-dihydro-4H-thieno[2,3-c]pyrrol-2-yl)methyl)-4-oxo-3-(((R)-1- | 93 |
| (o-tolyl)ethyl)amino)-4,6,7,8-tetrahydropyrrolo[1,2-a]pyrazine-6- | |
| carboxamide | |
| (S)-N-((5,6-dihydro-4H-thieno[2,3-c]pyrrol-2-yl)methyl)-4-oxo-3-(((S)-1- | 94 |
| (o-tolyl)ethyl)amino)-4,6,7,8-tetrahydropyrrolo[1,2-a]pyrazine-6- | |
| carboxamide | |
| (S)-N-((5,6-dihydro-4H-thieno[2,3-c]pyrrol-2-yl)methyl)-4-oxo-3-(((S)-1- | 95 |
| (m-tolyl)ethyl)amino)-4,6,7,8-tetrahydropyrrolo[1,2-a]pyrazine-6- | |
| carboxamide | |
| (S)-N-((5,6-dihydro-4H-thieno[2,3-c]pyrrol-2-yl)methyl)-3-((3,5- | 96 |
| dimethylbenzyl)amino)-4-oxo-4,6,7,8-tetrahydropyrrolo[1,2-a]pyrazine-6- | |
| carboxamide | |

### Example 6

### Preparation of (S)—N-((1H-pyrrolo[3,2-c]pyridin-2-yl)methyl)-3-((3,5-dimethylbenzyl)amino)-4-oxo-4,6,7,8-tetrahydropyrrolo[1,2-a]pyrazine-6-carboxamide trifluoroacetate

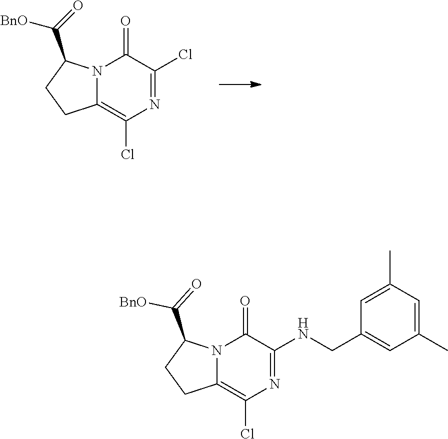 [SMILES: O=C(OCc1ccccc1)[C@@H]1CCc2c(Cl)nc(Cl)c(=O)n21 ; Cc1cc(C)cc(CNc2nc(Cl)c3n(c2=O)[C@H](C(=O)OCc2ccccc2)CC3)c1]

Step 1: Benzyl (S)-1,3-dichloro-4-oxo-4,6,7,8-tetrahydropyrrolo[1,2-a]pyrazine-6-carboxylate (125 mg, 0.37 mmol), (3-(methylsulfonyl)phenyl)methanamine (82 mg, 0.44 mmol), and DIEA (193 μL, 1.11 mmol) were added to a flask charged with 3.5 mL of MeCN. The reaction mixture was stirred at 80° C. overnight. The crude material was concentrated and purified by column chromatography with heptane-EtOAc. Pure fractions were isolated to afford benzyl (S)-1-chloro-3-((3-(methylsulfonyl)benzyl)amino)-4-oxo-4,6,7,8-tetrahydropyrrolo[1,2-a]pyrazine-6-carboxylate (101 mg, 56% yield) as a yellow solid.

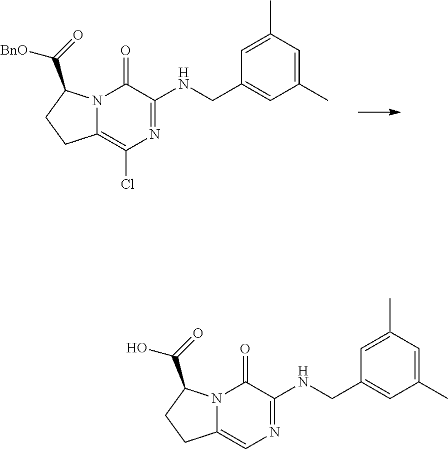 [SMILES: Cc1cc(C)cc(CNc2nc(Cl)c3n(c2=O)[C@H](C(=O)OCc2ccccc2)CC3)c1 ; Cc1cc(C)cc(CNc2ncc3n(c2=O)[C@H](C(=O)O)CC3)c1]

Step 2: Benzyl (S)-1-chloro-3-((3-(methylsulfonyl)benzyl)amino)-4-oxo-4,6,7,8-tetrahydropyrrolo[1,2-a]pyrazine-6-carboxylate (100 mg, 228 μmol), 10% Pd/C (24 mg), and DIEA (80 μL, 457 μmol) were added to a flask charged with 2 mL of EtOH under N₂. The flask was placed under vacuum and backfilled with H₂. This was repeated three times. The mixture was stirred under 1 atm of H₂ overnight. The crude material was filtered and concentrated to afford impure (S)-3-((3,5-dimethylbenzyl)amino)-4-oxo-4,6,7,8-tetrahydropyrrolo[1,2-a]pyrazine-6-carboxylic acid (25 mg). The product was taken to the next step without further purification.

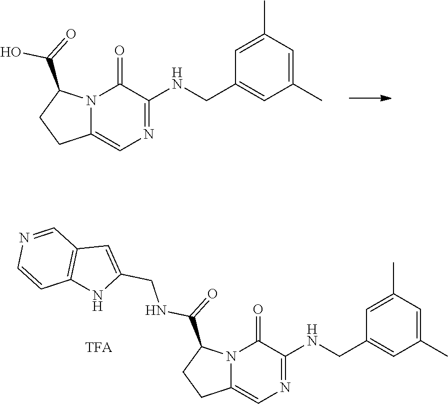 [SMILES: Cc1cc(C)cc(CNc2ncc3n(c2=O)[C@H](C(=O)O)CC3)c1 ; Cc1cc(C)cc(CNc2ncc3n(c2=O)[C@H](C(=O)NCc2cc4cnccc4[nH]2)CC3)c1]

Step 3: Crude (S)-3-((3,5-dimethylbenzyl)amino)-4-oxo-4,6,7,8-tetrahydropyrrolo[1,2-a]pyrazine-6-carboxylic acid (25 mg, ˜72 μmol), (1H-pyrrolo[3,2-c]pyridin-2-yl)methanamine (16 mg, 0.10 mmol), HATU (41 mg, 108 μmol) and DIEA (40 μL, 0.22 mmol) were added to a 20 mL vial charged with DMF (0.7 mL). The reaction was stirred at room temperature for 1 hour. The crude material was purified on an amine column with heptane then DCM-MeOH gradient elution. Semi-pure fractions were combined then re-purified by prep HPLC (ACN/H₂O+0.1% TFA). The pure product was collected to afford the white solid (S)—N-((1H-pyrrolo[3,2-c]pyridin-2-yl)methyl)-3-((3,5-dimethylbenzyl)amino)-4-oxo-4,6,7,8-tetrahydropyrrolo[1,2-a]pyrazine-6-carboxamide trifluoroacetate (3.6 mg, 10% yield).

### Example 7

### Preparation of (S)—N-((6-amino-2-methylpyridin-3-yl)methyl)-1-chloro-3-((3-(methylsulfonyl)benzyl)amino)-4-oxo-4,6,7,8-tetrahydropyrrolo[1,2-a]pyrazine-6-carboxamide trifluoroacetate and (S)—N-((6-amino-2-methylpyridin-3-yl)methyl)-3-((3-(methylsulfonyl)benzyl)amino)-4-oxo-4,6,7,8-tetrahydropyrrolo[1,2-a]pyrazine-6-carboxamide trifluoroacetate

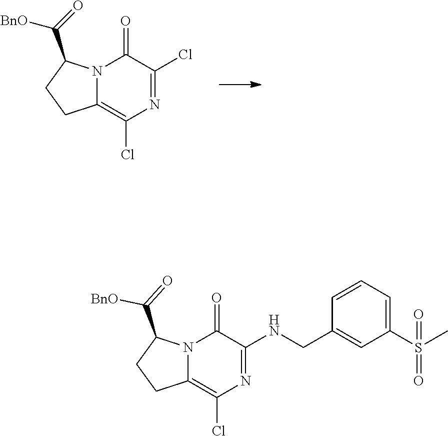 [SMILES: O=C(OCc1ccccc1)[C@@H]1CCc2c(Cl)nc(Cl)c(=O)n21 ; CS(=O)(=O)c1cccc(CNc2nc(Cl)c3n(c2=O)[C@H](C(=O)OCc2ccccc2)CC3)c1]

Step 1: Benzyl (S)-1,3-dichloro-4-oxo-4,6,7,8-tetrahydropyrrolo[1,2-a]pyrazine-6-carboxylate (200 mg, 589 μmol), (3,5-dimethylphenyl)methanamine (95 mg, 707 μmol), and DIEA (137 μL, 1.76 mmol) were added to a flask charged with MeCN (0.2 M). The reaction mixture was stirred for 1 hour at 70° C. and then overnight at 80° C. The crude material was concentrated and purified by column chromatography with a heptane-EtOAc gradient. Pure fractions were isolated to afford benzyl (S)-1-chloro-3-((3-(methylsulfonyl)benzyl)amino)-4-oxo-4,6,7,8-tetrahydropyrrolo[1,2-a]pyrazine-6-carboxylate (196 mg, 76% yield) as a yellow solid.

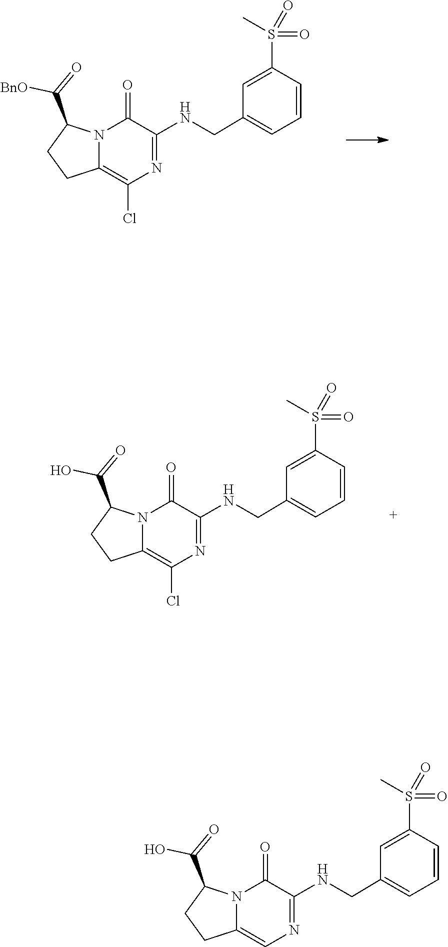 [SMILES: CS(=O)(=O)c1cccc(CNc2nc(Cl)c3n(c2=O)[C@H](C(=O)OCc2ccccc2)CC3)c1 ; CS(=O)(=O)c1cccc(CNc2nc(Cl)c3n(c2=O)[C@H](C(=O)O)CC3)c1 ; CS(=O)(=O)c1cccc(CNc2ncc3n(c2=O)[C@H](C(=O)O)CC3)c1]

Step 2: Benzyl (S)-1-chloro-3-((3-(methylsulfonyl)benzyl)amino)-4-oxo-4,6,7,8-tetrahydropyrrolo[1,2-a]pyrazine-6-carboxylate (100 mg, 228 μmol), 10% Pd/C (23 mg), and DIEA (73 μL, 421 μmol) were added to a flask charged with 2 mL of EtOH under N₂. The flask was placed under vacuum and backfilled with H₂ gas. This was repeated three times. The mixture was allowed to stir under 1 atm H₂ overnight. The crude material was filtered and concentrated to afford a mixture of (S)-1-chloro-3-((3-(methylsulfonyl)benzyl)amino)-4-oxo-4,6,7,8-tetrahydropyrrolo[1,2-a]pyrazine-6-carboxylic acid and (S)-3-((3-(methylsulfonyl)benzyl)amino)-4-oxo-4,6,7,8-tetrahydropyrrolo[1,2-a]pyrazine-6-carboxylic acid. The product was carried to the next step without further purification.

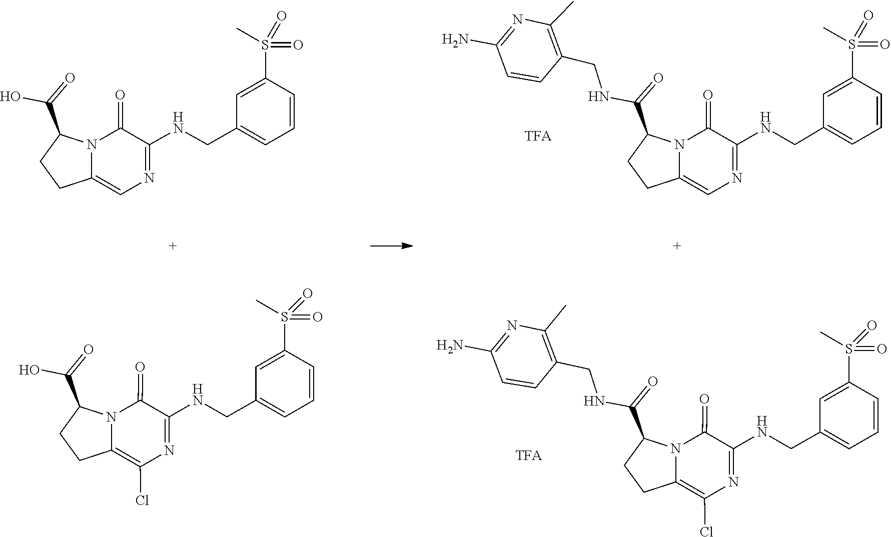 [SMILES: CS(=O)(=O)c1cccc(CNc2ncc3n(c2=O)[C@H](C(=O)O)CC3)c1 ; Cc1nc(N)ccc1CNC(=O)[C@@H]1CCc2cnc(NCc3cccc(S(C)(=O)=O)c3)c(=O)n21 ; CS(=O)(=O)c1cccc(CNc2nc(Cl)c3n(c2=O)[C@H](C(=O)O)CC3)c1 ; Cc1nc(N)ccc1CNC(=O)[C@@H]1CCc2c(Cl)nc(NCc3cccc(S(C)(=O)=O)c3)c(=O)n21]

Step 3: A mixture of (S)-1-chloro-3-((3-(methylsulfonyl)benzyl)amino)-4-oxo-4,6,7,8-tetrahydropyrrolo[1,2-a]pyrazine-6-carboxylic acid and (S)-3-((3-(methylsulfonyl)benzyl)amino)-4-oxo-4,6,7,8-tetrahydropyrrolo[1,2-a]pyrazine-6-carboxylic acid (78.9 mg, 0.21 mmol), 5-(aminomethyl)-6-methylpyridin-2-amine (38 mg, 0.28 mmol), HATU (100 mg, 0.28 mmol), and DIEA (96 μL, 0.55 mmol) were added to a 20 mL vial charged with DMF (2 mL). The reaction was allowed to stir at room temperature for 4 h. The crude material was purified by prep HPLC to afford the separated products (S)—N-((6-amino-2-methylpyridin-3-yl)methyl)-1-chloro-3-((3-(methylsulfonyl)benzyl)amino)-4-oxo-4,6,7,8-tetrahydropyrrolo[1,2-a]pyrazine-6-carboxamide trifluoroacetate (13.5 mg) and (S)—N-((6-amino-2-methylpyridin-3-yl)methyl)-3-((3-(methylsulfonyl)benzyl)amino)-4-oxo-4,6,7,8-tetrahydropyrrolo[1,2-a]pyrazine-6-carboxamide trifluoroacetate (65.8 mg).

### Example 8

### Preparation of (S)—N-((1H-pyrrolo[3,2-c]pyridin-2-yl)methyl)-1-chloro-3-(cyclobutylamino)-4-oxo-4,6,7,8-tetrahydropyrrolo[1,2-a]pyrazine-6-carboxamide trifluoroacetate

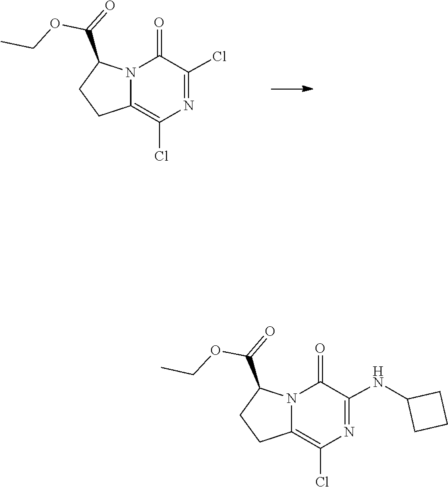 [SMILES: CCOC(=O)[C@@H]1CCc2c(Cl)nc(Cl)c(=O)n21 ; CCOC(=O)[C@@H]1CCc2c(Cl)nc(NC3CCC3)c(=O)n21]

Step 1: Ethyl (S)-1,3-dichloro-4-oxo-4,6,7,8-tetrahydropyrrolo[1,2-a]pyrazine-6-carboxylate (57 mg, 0.21 mmol), cyclobutylamine hydrochloride (43 mg, 0.41 mmol), and DIEA (105 μL, 0.63 mmol) were dissolved in MeCN (2 mL). The mixture was stirred overnight at 80° C. The reaction mixture was filtered and purified by column chromatography (heptane-EtOAc) to afford ethyl (S)-1-chloro-3-(cyclobutylamino)-4-oxo-4,6,7,8-tetrahydropyrrolo[1,2-a]pyrazine-6-carboxylate (54 mg, 85% yield) as a white solid.

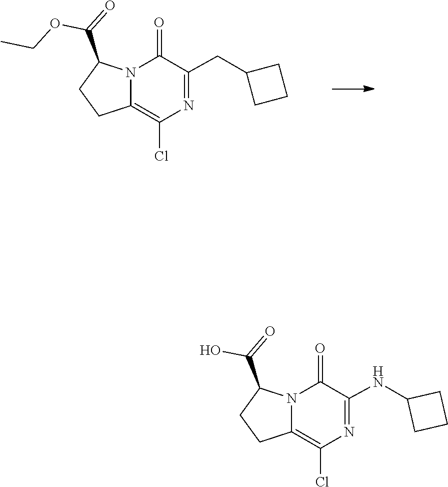 [SMILES: CCOC(=O)[C@@H]1CCc2c(Cl)nc(CC3CCC3)c(=O)n21 ; O=C(O)[C@@H]1CCc2c(Cl)nc(NC3CCC3)c(=O)n21]

Step 2: Ethyl (S)-1-chloro-3-(cyclobutylamino)-4-oxo-4,6,7,8-tetrahydropyrrolo[1,2-a]pyrazine-6-carboxylate (54 mg, 0.17 mmol) was dissolved in THF (2 mL). The mixture was sonicated, followed by the addition of 1 N aq. LiOH (1 mL). The reaction mixture was stirred for 5 h. The crude reaction mixture was concentrated, acidified with aqueous 10% HCl, and filtered to afford (S)-1-chloro-3-(cyclobutylamino)-4-oxo-4,6,7,8-tetrahydropyrrolo[1,2-a]pyrazine-6-carboxylic acid (quantitative yield) as a white solid.

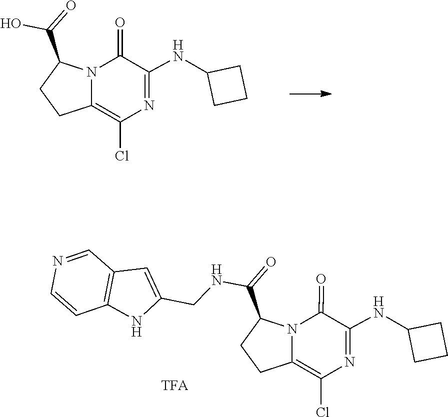 [SMILES: O=C(O)[C@@H]1CCc2c(Cl)nc(NC3CCC3)c(=O)n21 ; O=C(NCc1cc2cnccc2[nH]1)[C@@H]1CCc2c(Cl)nc(NC3CCC3)c(=O)n21]

Step 3: (S)-1-chloro-3-(cyclobutylamino)-4-oxo-4,6,7,8-tetrahydropyrrolo[1,2-a]pyrazine-6-carboxylic acid (58 mg, 0.20 mmol) and (1H-pyrrolo[3,2-c]pyridin-2-yl)methanamine (35 mg, 0.24 mmol) were added to DMF (˜3 mL). The reaction mixture was cooled to 0° C., then HBTU (84 mg, 0.22 mmol) and DIEA (105 uL, 0.60 mmol) were added. The reaction was allowed to stir for 2 d, at which time it was concentrated and purified by an amine column (DCM-MeOH). The semi-pure material was then purified by prep HPLC to afford (S)—N-((1H-pyrrolo[3,2-c]pyridin-2-yl)methyl)-1-chloro-3-(cyclobutylamino)-4-oxo-4,6,7,8-tetrahydropyrrolo[1,2-a]pyrazine-6-carboxamide trifluoroacetate (23.6 mg, 22% yield) as a white solid.

The following compound was prepared according to the foregoing procedures with the appropriate amine:

| | Cmp |
|---|---|
| Compound Name | No. |
| (S)-N-((1H-pyrrolo[3,2-c]pyridin-2-yl)methyl)-1-chloro-4-oxo-3- | 101 |
| (((tetrahydro-2H-pyran-4-yl)methyl)amino)-4,6,7,8-tetrahydropyrrolo[1,2- | |
| a]pyrazine-6-carboxamide trifluoroacetate | |

### Example 9

### Preparation of (S)—N-((1H-pyrrolo[3,2-c]pyridin-2-yl)methyl)-3-(benzylamino)-4-oxo-4,6,7,8-tetrahydropyrrolo[1,2-a]pyrazine-6-carboxamide

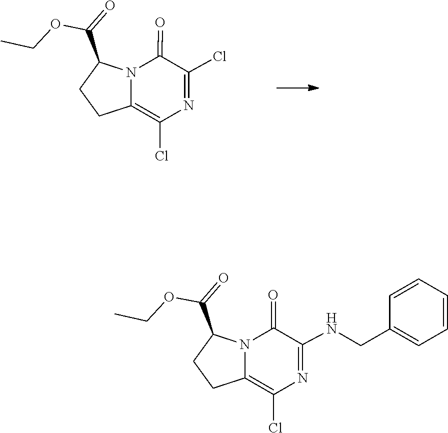 [SMILES: CCOC(=O)[C@@H]1CCc2c(Cl)nc(Cl)c(=O)n21 ; CCOC(=O)[C@@H]1CCc2c(Cl)nc(NCc3ccccc3)c(=O)n21]

Step 1: Ethyl (S)-1,3-dichloro-4-oxo-4,6,7,8-tetrahydropyrrolo[1,2-a]pyrazine-6-carboxylate (46 mg, 0.17 mmol), phenylmethanamine (36 μL, 0.33 mmol), and DIEA (89 μL, 0.50 mmol) were dissolved in MeCN (2 mL). The mixture was stirred for three d at 80° C. The reaction mixture was concentrated and purified by chromatography (heptane-EtOAc) to afford ethyl (S)-3-(benzylamino)-1-chloro-4-oxo-4,6,7,8-tetrahydropyrrolo[1,2-a]pyrazine-6-carboxylate (16.7 mg, 57% yield) as a white solid.

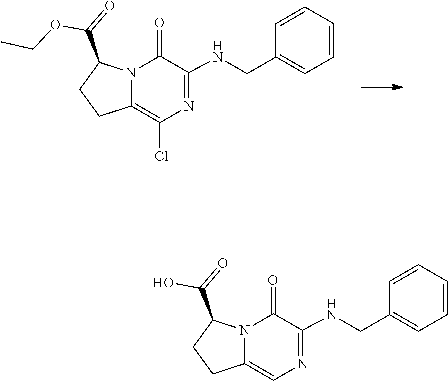 [SMILES: CCOC(=O)[C@@H]1CCc2c(Cl)nc(NCc3ccccc3)c(=O)n21 ; O=C(O)[C@@H]1CCc2cnc(NCc3ccccc3)c(=O)n21]

Step 2: Ethyl (S)-3-(benzylamino)-1-chloro-4-oxo-4,6,7,8-tetrahydropyrrolo[1,2-a]pyrazine-6-carboxylate (52 mg, 0.15 mmol) was dissolved in EtOAc (3 mL). The mixture was sonicated followed by the addition of 1 N NaOH (0.6 mL) and 10% Pd/C (16 mg). The mixture was put under vacuum and backfilled with H₂. This was repeated three times. The reaction was stirred under 1 atm H₂ for 16 h. The crude reaction mixture was acidified with 10% H₂SO₄ and concentrated to afford (S)-3-(benzylamino)-4-oxo-4,6,7,8-tetrahydropyrrolo[1,2-a]pyrazine-6-carboxylic acid (yield assumed quantitative).

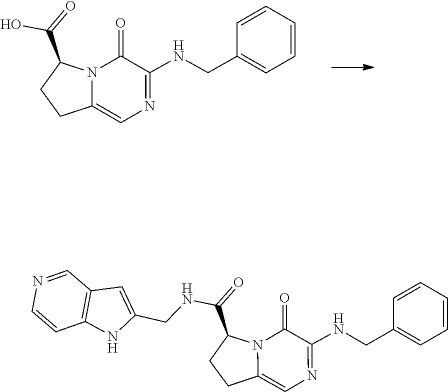 [SMILES: O=C(O)[C@@H]1CCc2cnc(NCc3ccccc3)c(=O)n21 ; O=C(NCc1cc2cnccc2[nH]1)[C@@H]1CCc2cnc(NCc3ccccc3)c(=O)n21]

Step 3: The above compound was synthesized according to General Amide Coupling Procedure A, except that only a single amine column chromatography was performed to afford (S)—N-((1H-pyrrolo[3,2-c]pyridin-2-yl)methyl)-3-(benzylamino)-4-oxo-4,6,7,8-tetrahydropyrrolo[1,2-a]pyrazine-6-carboxamide (7.8 mg, 13% yield).

The following compounds were prepared according to the foregoing procedures with the appropriate amines:

| | Cmp |
|---|---|
| Compound Name | No. |
| (S)-3-(benzylamino)-N-((6-methyl- | 103 |
| 1H-pyrrolo[3,2-c]pyridin-2-yl)methyl)- | |
| 4-oxo-4,6,7,8-tetrahydropyrrolo[1,2- | |
| a]pyrazine-6-carboxamide | |
| (S)-N-((6-amino-2-methylpyridin-3- | 104 |
| yl)methyl)-3-(benzylamino)-4-oxo- | |
| 4,6,7,8-tetrahydropyrrolo[1,2- | |
| a]pyrazine-6-carboxamide | |

### Example 10

### Preparation of (S)—N-((1H-pyrrolo[3,2-c]pyridin-2-yl)methyl)-3-(benzylamino)-1-chloro-4-oxo-4,6,7,8-tetrahydropyrrolo[1,2-a]pyrazine-6-carboxamide

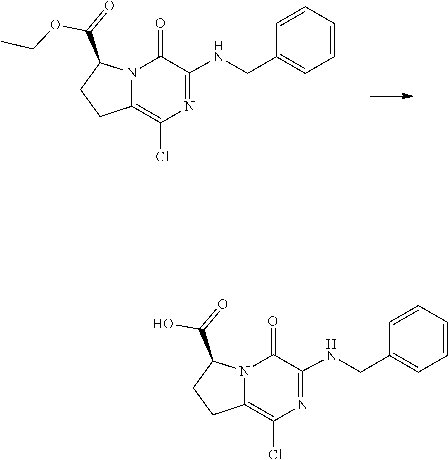 [SMILES: CCOC(=O)[C@@H]1CCc2c(Cl)nc(NCc3ccccc3)c(=O)n21 ; O=C(O)[C@@H]1CCc2c(Cl)nc(NCc3ccccc3)c(=O)n21]

Step 1: Ethyl (S)-3-(benzylamino)-1-chloro-4-oxo-4,6,7,8-tetrahydropyrrolo[1,2-a]pyrazine-6-carboxylate (50 mg, 0.14 mmol) was dissolved in THF (2 mL). The mixture was sonicated followed by the addition of 1 N LiOH (1 mL). The reaction was stirred for 2.5 h at which time the mixture was concentrated, acidified with 2 N HCl and extracted twice with EtOAc. The organic layers were combined, washed with brine, and dried over Na₂SO₄ to afford (S)-3-(benzylamino)-1-chloro-4-oxo-4,6,7,8-tetrahydropyrrolo[1,2-a]pyrazine-6-carboxylic acid (quantitative yield) as a brown oil.

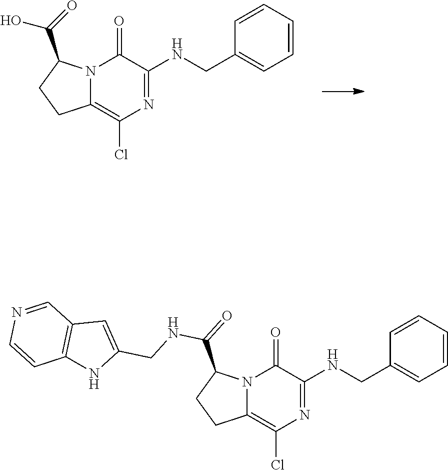 [SMILES: O=C(O)[C@@H]1CCc2c(Cl)nc(NCc3ccccc3)c(=O)n21 ; O=C(NCc1cc2cnccc2[nH]1)[C@@H]1CCc2c(Cl)nc(NCc3ccccc3)c(=O)n21]

Step 2: (S)-3-(benzylamino)-1-chloro-4-oxo-4,6,7,8-tetrahydropyrrolo[1,2-a]pyrazine-6-carboxylic acid (47 mg, 0.14 mmol), (1H-pyrrolo[3,2-c]pyridin-2-yl)methanamine (25 mg, 0.17 mmol), DCC (31 mg, 0.15 mmol), and NHS (20 mg, 0.17 mmol) were added to DMF (0.5 mL) and DCM (1.5 mL). The reaction was allowed to stir until completion, then was concentrated and purified by an amine column eluting with a DCM-MeOH gradient to afford (S)—N-((1H-pyrrolo[3,2-c]pyridin-2-yl)methyl)-3-(benzylamino)-1-chloro-4-oxo-4,6,7,8-tetrahydropyrrolo[1,2-a]pyrazine-6-carboxamide (31 mg, 50% yield) as a white solid.

The following compound was prepared according to the foregoing procedures with the appropriate amine:

| | Cmp |
|---|---|
| Compound Name | No. |
| (S)-N-((1H-pyrrolo[3,2-c]pyridin- | 106 |
| 2-yl)methyl)-1-chloro-4-oxo-3-((1- | |
| phenylcyclopropyl)amino)-4,6,7,8- | |
| tetrahydropyrrolo[1,2-a]pyrazine-6- | |
| carboxamide | |

The following compound was prepared according to the foregoing procedures with the appropriate amine, except that Amide Coupling Procedure A was used in the final coupling step:

| | Cmp |
|---|---|
| Compound Name | No. |
| (S)-3-(benzylamino)-1-chloro-N- | 107 |
| (5-chloro-2-(1H-tetrazol-1-yl)benzyl)-4- | |
| oxo-4,6,7,8-tetrahydropyrrolo[1,2- | |
| a]pyrazine-6-carboxamide | |

### Example 11

### Preparation of (S)—N-((6,7-dihydro-5H-pyrrolo[3,4-b]pyridin-3-yl)methyl)-3-((3,5-dimethylbenzyl)amino)-4-oxo-4,6,7,8-tetrahydropyrrolo[1,2-a]pyrazine-6-carboxamide

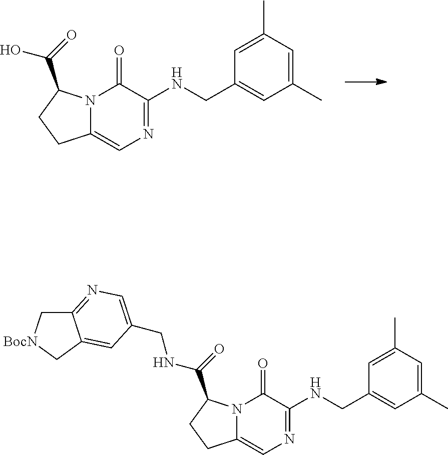 [SMILES: Cc1cc(C)cc(CNc2ncc3n(c2=O)[C@H](C(=O)O)CC3)c1 ; Cc1cc(C)cc(CNc2ncc3n(c2=O)[C@H](C(=O)NCc2cnc4c(c2)CN(C(=O)OC(C)(C)C)C4)CC3)c1]

Step 1: The above compound was synthesized according to General Amide Coupling Procedure A, except that it was purified by chromatography (DCM-MeOH) to afford tert-butyl (S)-3-((3-((3,5-dimethylbenzyl)amino)-4-oxo-4,6,7,8-tetrahydropyrrolo[1,2-a]pyrazine-6-carboxamido)methyl)-5,7-dihydro-6H-pyrrolo[3,4-b]pyridine-6-carboxylate (67 mg. 54% yield).

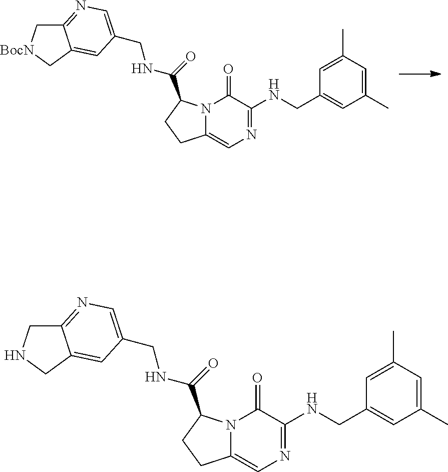 [SMILES: Cc1cc(C)cc(CNc2ncc3n(c2=O)[C@H](C(=O)NCc2cnc4c(c2)CN(C(=O)OC(C)(C)C)C4)CC3)c1 ; Cc1cc(C)cc(CNc2ncc3n(c2=O)[C@H](C(=O)NCc2cnc4c(c2)CNC4)CC3)c1]

Step 2: The above compound was synthesized according to General Boc Removal Procedure A except that purification consisted of an additional silica gel column using DCM-MeOH (with 7M NH₃) to afford (S)—N-((6,7-dihydro-5H-pyrrolo[3,4-b]pyridin-3-yl)methyl)-3-((3,5-dimethylbenzyl)amino)-4-oxo-4,6,7,8-tetrahydropyrrolo[1,2-a]pyrazine-6-carboxamide (12 mg, 41% yield).

The following compound was prepared according to the foregoing procedures with the appropriate amine, except that prep HPLC was used for final purification:

| | Cmp |
|---|---|
| Compound Name | No. |
| (S)-N-((6-(aminomethyl)pyridin-3-yl)methyl)-3-((3,5- | 109 |
| dimethylbenzyl)amino)-4-oxo-4,6,7,8- | |
| tetrahydropyrrolo[1,2-a]pyrazine-6- | |
| carboxamide trifluoroacetate | |

### Example 12

### Preparation of (S)—N-((1H-pyrrolo[3,2-c]pyridin-2-yl)methyl)-1-chloro-4-oxo-3-((1-(m-tolyl)cyclopropyl)amino)-4,6,7,8-tetrahydropyrrolo[1,2-a]pyrazine-6-carboxamide trifluoroacetate and (S)—N-((1H-pyrrolo[3,2-c]pyridin-2-yl)methyl)-4-oxo-3-((1-(m-tolyl)cyclopropyl)amino)-4,6,7,8-tetrahydropyrrolo[1,2-a]pyrazine-6-carboxamide trifluoroacetate

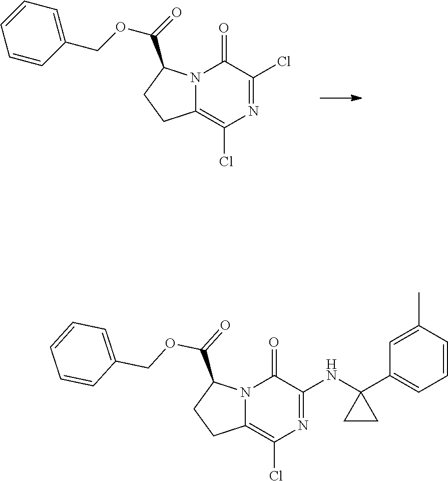 [SMILES: O=C(OCc1ccccc1)[C@@H]1CCc2c(Cl)nc(Cl)c(=O)n21 ; Cc1cccc(C2(Nc3nc(Cl)c4n(c3=O)[C@H](C(=O)OCc3ccccc3)CC4)CC2)c1]

Step 1: Benzyl (S)-1,3-dichloro-4-oxo-4,6,7,8-tetrahydropyrrolo[1,2-a]pyrazine-6-carboxylate (34 mg, 0.1 mmol), 1-(m-tolyl)cyclopropan-1-amine hydrochloride (20 mg, 0.11 mmol), and DIEA (50 μL, 0.3 mmol) were dissolved in ACN (˜2 mL) and heated to 160° C. under microwave irradiation for 15 minutes. The reaction was repeated twice more with dioxane as the solvent for one example and DMF as the solvent for another. The three reactions were combined and purified by column chromatography to afford benzyl (S)-1-chloro-4-oxo-3-((1-(m-tolyl)cyclopropyl)amino)-4,6,7,8-tetrahydropyrrolo[1,2-a]pyrazine-6-carboxylate (73 mg, 57% yield).

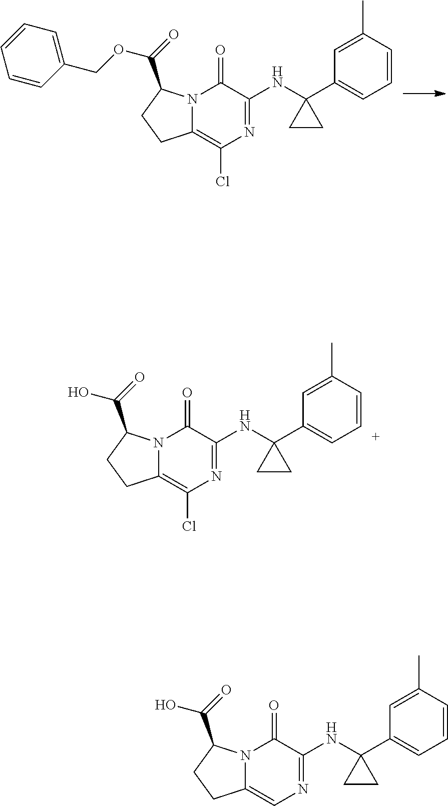 [SMILES: Cc1cccc(C2(Nc3nc(Cl)c4n(c3=O)[C@H](C(=O)OCc3ccccc3)CC4)CC2)c1 ; Cc1cccc(C2(Nc3nc(Cl)c4n(c3=O)[C@H](C(=O)O)CC4)CC2)c1 ; Cc1cccc(C2(Nc3ncc4n(c3=O)[C@H](C(=O)O)CC4)CC2)c1]

Step 2: Benzyl (S)-1-chloro-4-oxo-3-((1-(m-tolyl)cyclopropyl)amino)-4,6,7,8-tetrahydropyrrolo[1,2-a]pyrazine-6-carboxylate (73 mg, 0.17 mmol) and 10 mol % Pd/C (0.1 equiv) were added to a vial under 1 atm H₂. The reaction was stirred for 4 d. The crude material was filtered through a PTFE microfilter and concentrated to afford a mixture of two products; (S)-1-chloro-4-oxo-3-((1-(m-tolyl)cyclopropyl)amino)-4,6,7,8-tetrahydropyrrolo[1,2-a]pyrazine-6-carboxylic acid and (S)-4-oxo-3-((1-(m-tolyl)cyclopropyl)amino)-4,6,7,8-tetrahydropyrrolo[1,2-a]pyrazine-6-carboxylic acid (quantitative yield).

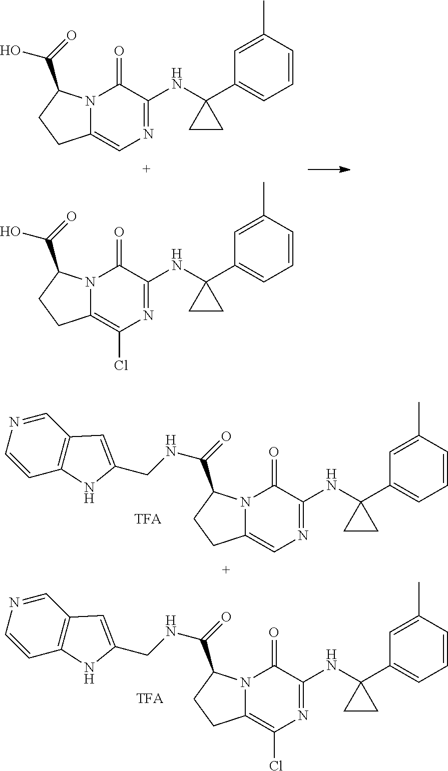 [SMILES: Cc1cccc(C2(Nc3ncc4n(c3=O)[C@H](C(=O)O)CC4)CC2)c1 ; Cc1cccc(C2(Nc3nc(Cl)c4n(c3=O)[C@H](C(=O)O)CC4)CC2)c1 ; Cc1cccc(C2(Nc3ncc4n(c3=O)[C@H](C(=O)NCc3cc5cnccc5[nH]3)CC4)CC2)c1 ; Cc1cccc(C2(Nc3nc(Cl)c4n(c3=O)[C@H](C(=O)NCc3cc5cnccc5[nH]3)CC4)CC2)c1]

Step 3: A mixture of (S)-1-chloro-4-oxo-3-((1-(m-tolyl)cyclopropyl)amino)-4,6,7,8-tetrahydropyrrolo[1,2-a]pyrazine-6-carboxylic acid and (S)-4-oxo-3-((1-(m-tolyl)cyclopropyl)amino)-4,6,7,8-tetrahydropyrrolo[1,2-a]pyrazine-6-carboxylic acid (61 mg, 0.17 mmol), (1H-pyrrolo[3,2-c]pyridin-2-yl)methanamine (30 mg, 0.20 mmol), HATU (71 mg, 0.19 mmol), and DIEA (89 uL, 0.50 mmol) were added to DMF (2 mL). Upon completion of the reaction, the crude material was purified by prep HPLC to afford (S)—N-((1H-pyrrolo[3,2-c]pyridin-2-yl)methyl)-1-chloro-4-oxo-3-((1-(n-tolyl)cyclopropyl)amino)-4,6,7,8-tetrahydropyrrolo[1,2-a]pyrazine-6-carboxamide trifluoroacetate (7.7 mg, 18% yield) and (S)—N-((1H-pyrrolo[3,2-c]pyridin-2-yl)methyl)-4-oxo-3-((1-(m-tolyl)cyclopropyl)amino)-4,6,7,8-tetrahydropyrrolo[1,2-a]pyrazine-6-carboxamide trifluoroacetate (1.0 mg, 2% yield).

The following compounds were prepared according to the foregoing procedures with the appropriate amines:

| | Cmp |
|---|---|
| Compound Name | No. |
| (S)-N-((1H-pyrrolo[3,2-c]pyridin- | 112 |
| 2-yl)methyl)-1-chloro-3- | |
| ((cyclohexylmethyl)amino)-4-oxo- | |
| 4,6,7,8-tetrahydropyrrolo[1,2- | |
| alpyrazine-6-carboxamide trifluoroacetate | |
| (S)-N-((1H-pyrrolo[3,2- | 113 |
| c]pyridin-2-yl)methyl)-3- | |
| ((cyclohexylmethyl)amino)-4-oxo- | |
| 4,6,7,8-tetrahydropyrrolo[1,2- | |
| alpyrazine-6-carboxamide trifluoroacetate | |

The following compounds were prepared according to the foregoing procedures with the appropriate amines, except that an amine column was performed before prep HPLC during purification of the final amide coupling step:

| | Cmp |
|---|---|
| Compound Name | No. |
| (S)-N-((1H-pyrrolo[3,2-c]pyridin- | 114 |
| 2-yl)methyl)-1-chloro-3-((3- | |
| methylbenzyl)amino)-4-oxo-4,6,7,8-tetrahydropyrrolo[1,2- | |
| a]pyrazine-6-carboxamide trifluoroacetate | |
| (S)-N-((1H-pyrrolo[3,2-c]pyridin- | 115 |
| 2-yl)methyl)-3-((3-methylbenzyl)amino)- | |
| 4-oxo-4,6,7,8-tetrahydropyrrolo[1,2- | |
| a]pyrazine-6-carboxamide trifluoroacetate | |

### Example 13

### Preparation of (S)—N-((6-amino-2-methylpyridin-3-yl)methyl)-4-oxo-3-(((tetrahydro-2H-pyran-4-yl)methyl)amino)-4,6,7,8-tetrahydropyrrolo[1,2-a]pyrazine-6-carboxamide

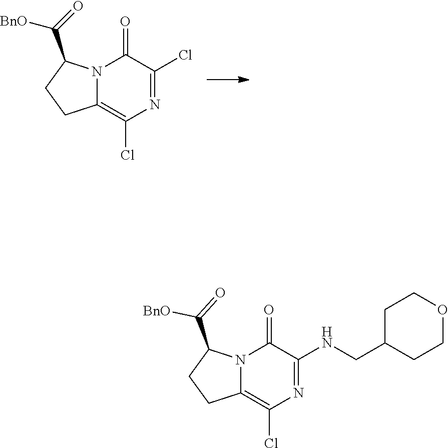 [SMILES: O=C(OCc1ccccc1)[C@@H]1CCc2c(Cl)nc(Cl)c(=O)n21 ; O=C(OCc1ccccc1)[C@@H]1CCc2c(Cl)nc(NCC3CCOCC3)c(=O)n21]

Step 1: The above compound was synthesized according to General Chloro Displacement Procedure A to afford benzyl (S)-1-chloro-4-oxo-3-(((tetrahydro-2H-pyran-4-yl)methyl)amino)-4,6,7,8-tetrahydropyrrolo[1,2-a]pyrazine-6-carboxylate (101 mg, 56% yield).

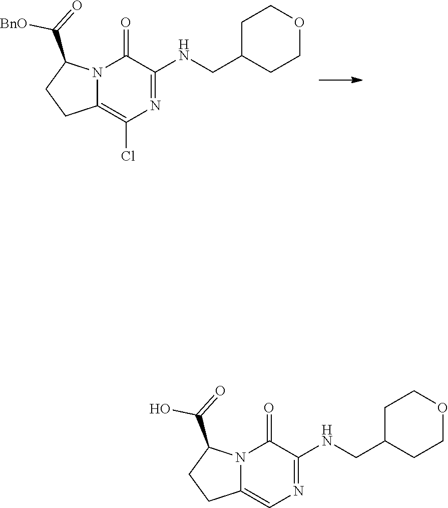 [SMILES: O=C(OCc1ccccc1)[C@@H]1CCc2c(Cl)nc(NCC3CCOCC3)c(=O)n21 ; O=C(O)[C@@H]1CCc2cnc(NCC3CCOCC3)c(=O)n21]

Step 2: Benzyl (S)-1-chloro-4-oxo-3-(((tetrahydro-2H-pyran-4-yl)methyl)amino)-4,6,7,8-tetrahydropyrrolo[1,2-a]pyrazine-6-carboxylate (29 mg, 70 μmol), 10% Pd/C (5 mg), and ammonium formate (13 mg, 0.20 mmol) were added to a flask charged with 2 mL of EtOH under N₂. The flask was placed under vacuum and backfilled with H₂ gas. This purge was repeated three times. The mixture was allowed to stir under 1 atm H₂ overnight. The crude material was filtered then concentrated to afford (S)-4-oxo-3-(((tetrahydro-2H-pyran-4-yl)methyl)amino)-4,6,7,8-tetrahydropyrrolo[1,2-a]pyrazine-6-carboxylic acid (quantitative yield).

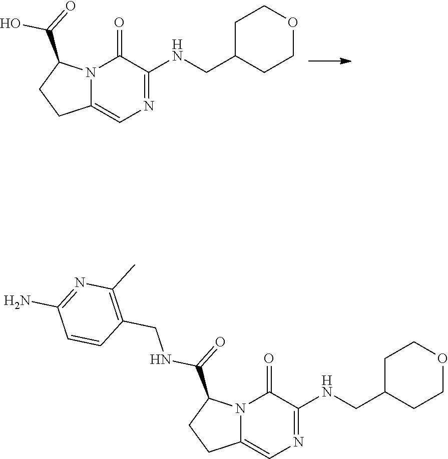 [SMILES: O=C(O)[C@@H]1CCc2cnc(NCC3CCOCC3)c(=O)n21 ; Cc1nc(N)ccc1CNC(=O)[C@@H]1CCc2cnc(NCC3CCOCC3)c(=O)n21]

Step 3: The above compound was synthesized according to General Amide Coupling Procedure A except that a single silica gel column was used to afford (S)—N-((6-amino-2-methylpyridin-3-yl)methyl)-4-oxo-3-(((tetrahydro-2H-pyran-4-yl)methyl)amino)-4,6,7,8-tetrahydropyrrolo[1,2-a]pyrazine-6-carboxamide (14.3 mg, 46% yield).

The following compound was prepared according to the foregoing procedures with the appropriate amine, except that prep HPLC was used to purify the final product:

| | Cmp |
|---|---|
| Compound Name | No. |
| (S)-N-((1H-pyrrolo[3,2-c]pyridin- | 117 |
| 2-yl)methyl)-4-oxo-3-((3- | |
| (trifluoromethyl)benzyl)amino)-4,6,7,8- | |
| tetrahydropyrrolo[1,2-a]pyrazine-6- | |
| carboxamide trifluoroacetate | |

### Example 14

### Preparation of (6S)—N-((6-amino-2-methylpyridin-3-yl)methyl)-1-chloro-4-oxo-3-(((tetrahydrofuran-3-yl)methyl)amino)-4,6,7,8-tetrahydropyrrolo[1,2-a]pyrazine-6-carboxamide

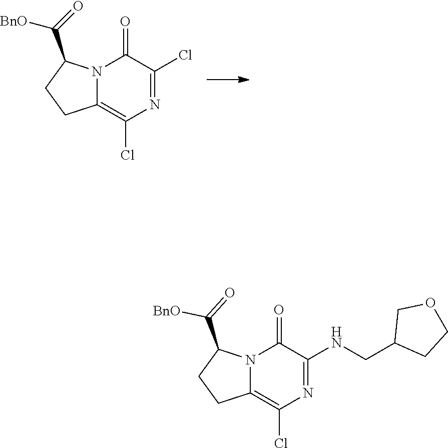 [SMILES: O=C(OCc1ccccc1)[C@@H]1CCc2c(Cl)nc(Cl)c(=O)n21 ; O=C(OCc1ccccc1)[C@@H]1CCc2c(Cl)nc(NCC3CCOC3)c(=O)n21]

Step 1: The above compound was synthesized according to General Chloro Displacement Procedure A to afford benzyl (6S)-1-chloro-4-oxo-3-(((tetrahydrofuran-3-yl)methyl)amino)-4,6,7,8-tetrahydropyrrolo[1,2-a]pyrazine-6-carboxylate (71 mg, 88% yield).

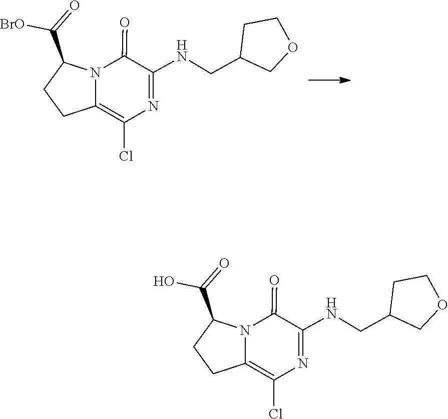

Step 2: Benzyl (6S)-1-chloro-4-oxo-3-(((tetrahydrofuran-3-yl)methyl)amino)-4,6,7,8-tetrahydropyrrolo[1,2-a]pyrazine-6-carboxylate (73 mg, 0.17 mmol) and 1 N aq. LiOH (1.8 mL) were added to a flask charged with THF (2 mL) and MeOH (1 mL). The reaction was stirred for 4 h at room temperature. The crude reaction mixture was concentrated, washed with 10% aq. KHSO₄, and filtered to afford (6S)-1-chloro-4-oxo-3-(((tetrahydrofuran-3-yl)methyl)amino)-4,6,7,8-tetrahydropyrrolo[1,2-a]pyrazine-6-carboxylic acid (quantitative yield).

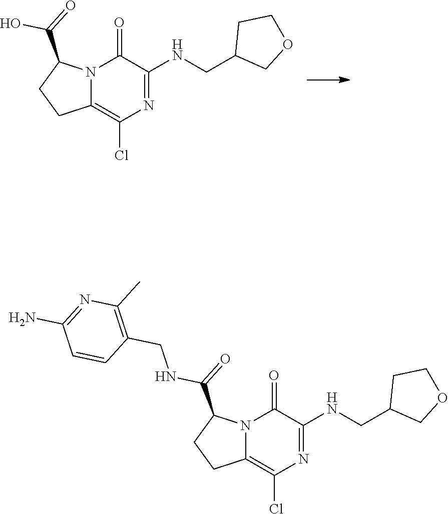 [SMILES: O=C(O)[C@@H]1CCc2c(Cl)nc(NCC3CCOC3)c(=O)n21 ; Cc1nc(N)ccc1CNC(=O)[C@@H]1CCc2c(Cl)nc(NCC3CCOC3)c(=O)n21]

Step 3: The above compound was synthesized according to General Amide Coupling Procedure A to afford (6S)—N-((6-amino-2-methylpyridin-3-yl)methyl)-1-chloro-4-oxo-3-(((tetrahydrofuranTHF-3-yl)methyl)amino)-4,6,7,8-tetrahydropyrrolo[1,2-a]pyrazine-6-carboxamide (15.4 mg, 21% yield).

### Example 15

### Preparation of (S)—N-((5,6-dihydro-4H-thieno[2,3-c]pyrrol-2-yl)methyl)-4-oxo-3-(3-phenylazetidin-1-yl)-4,6,7,8-tetrahydropyrrolo[1,2-a]pyrazine-6-carboxamide

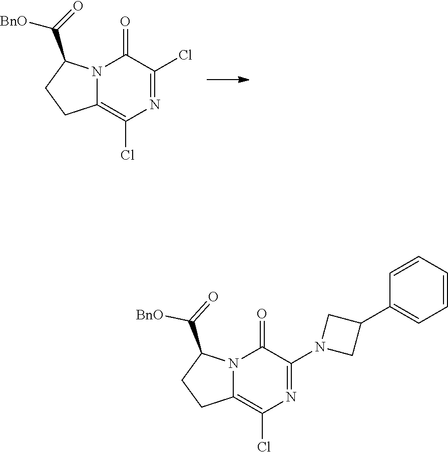 [SMILES: O=C(OCc1ccccc1)[C@@H]1CCc2c(Cl)nc(Cl)c(=O)n21 ; O=C(OCc1ccccc1)[C@@H]1CCc2c(Cl)nc(N3CC(c4ccccc4)C3)c(=O)n21]

Step 1: The above compound was synthesized according to General Chloro Displacement Procedure A to afford benzyl (S)-1-chloro-4-oxo-3-(3-phenylazetidin-1-yl)-4,6,7,8-tetrahydropyrrolo[1,2-a]pyrazine-6-carboxylate (35 mg, 38% yield).

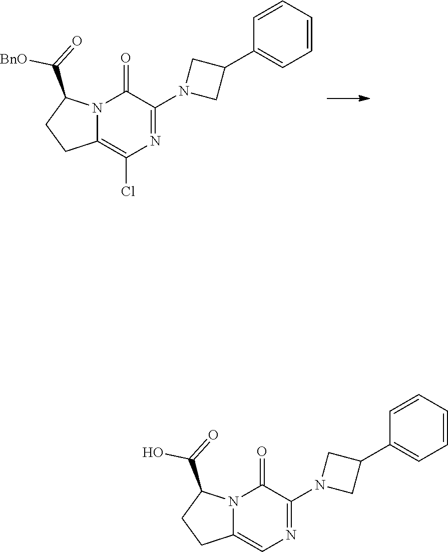 [SMILES: O=C(OCc1ccccc1)[C@@H]1CCc2c(Cl)nc(N3CC(c4ccccc4)C3)c(=O)n21 ; O=C(O)[C@@H]1CCc2cnc(N3CC(c4ccccc4)C3)c(=O)n21]

Step 2: The above compound was synthesized according to General Hydrogenolysis Procedure A, except that the reaction was stirred in a 1:1 mixture of EtOH and EtOAc to afford (S)-4-oxo-3-(3-phenylazetidin-1-yl)-4,6,7,8-tetrahydropyrrolo[1,2-a]pyrazine-6-carboxylic acid (quantitative yield).

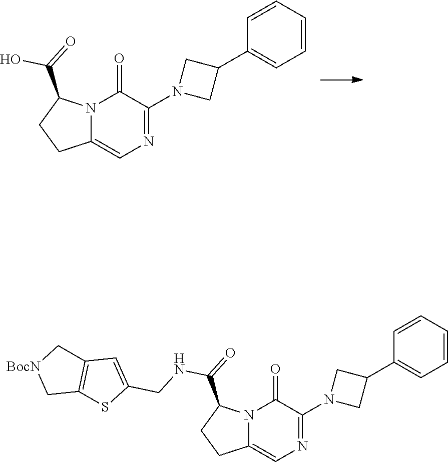 [SMILES: O=C(O)[C@@H]1CCc2cnc(N3CC(c4ccccc4)C3)c(=O)n21 ; CC(C)(C)OC(=O)N1Cc2cc(CNC(=O)[C@@H]3CCc4cnc(N5CC(c6ccccc6)C5)c(=O)n43)sc2C1]

Step 3: The above compound was synthesized according to General Amide Coupling Procedure A to afford tert-butyl (S)-2-((4-oxo-3-(3-phenylazetidin-1-yl)-4,6,7,8-tetrahydropyrrolo[1,2-a]pyrazine-6-carboxamido)methyl)-4,6-dihydro-5H-thieno[2,3-c]pyrrole-5-carboxylate (14 mg, 32% yield) as the pure product.

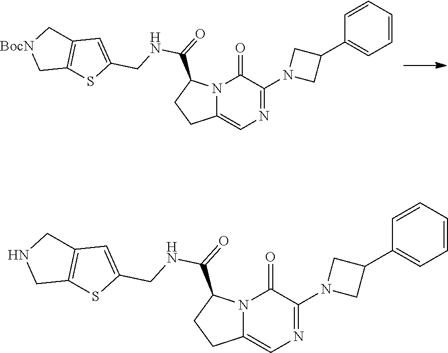 [SMILES: CC(C)(C)OC(=O)N1Cc2cc(CNC(=O)[C@@H]3CCc4cnc(N5CC(c6ccccc6)C5)c(=O)n43)sc2C1 ; O=C(NCc1cc2c(s1)CNC2)[C@@H]1CCc2cnc(N3CC(c4ccccc4)C3)c(=O)n21]

Step 4: A 25 mL round bottom flask was charged with tert-butyl (S)-2-((4-oxo-3-(3-phenylazetidin-1-yl)-4,6,7,8-tetrahydropyrrolo[1,2-a]pyrazine-6-carboxamido)methyl)-4,6-dihydro-5H-thieno[2,3-c]pyrrole-5-carboxylate (14 mg, 26 μmol) and 3 M HCl in MeOH (85 μL, 0.26 mmol) and stirred at room temperature overnight. The crude reaction mixture was concentrated twice, and purified by column chromatography eluting with a DCM-MeOH gradient. Pure fractions were collected and concentrated to afford (S)—N-((5,6-dihydro-4H-thieno[2,3-c]pyrrol-2-yl)methyl)-4-oxo-3-(3-phenylazetidin-1-yl)-4,6,7,8-tetrahydropyrrolo[1,2-a]pyrazine-6-carboxamide (4 mg, 30% yield).

### Example 16

### Preparation of (S)—N-((1H-pyrrolo[3,2-c]pyridin-2-yl)methyl)-3-(benzylamino)-1-(3,6-dihydro-2H-pyran-4-yl)-4-oxo-4,6,7,8-tetrahydropyrrolo[1,2-a]pyrazine-6-carboxamide

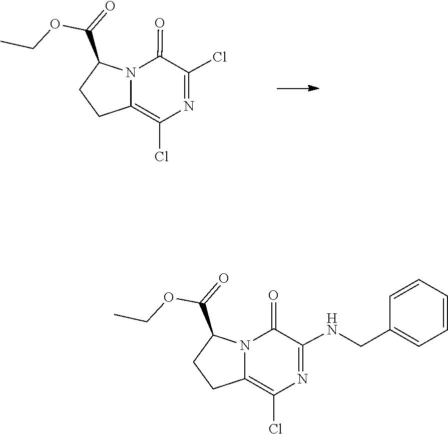 [SMILES: CCOC(=O)[C@@H]1CCc2c(Cl)nc(Cl)c(=O)n21 ; CCOC(=O)[C@@H]1CCc2c(Cl)nc(NCc3ccccc3)c(=O)n21]

Step 1: The above compound was synthesized according to Example 9, step 1.

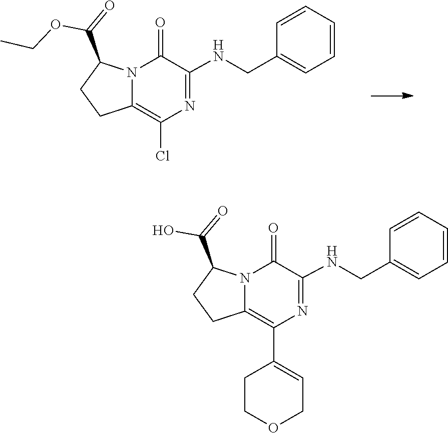 [SMILES: CCOC(=O)[C@@H]1CCc2c(Cl)nc(NCc3ccccc3)c(=O)n21 ; O=C(O)[C@@H]1CCc2c(C3=CCOCC3)nc(NCc3ccccc3)c(=O)n21]

Step 2: Ethyl (S)-3-(benzylamino)-1-chloro-4-oxo-4,6,7,8-tetrahydropyrrolo[1,2-a]pyrazine-6-carboxylate (35 mg, 0.1 mmol), 1-(3,6-dihydro-2H-pyran-4-yl)-3,3,4,4-tetramethylborolane (32 mg, 0.15 mmol), Pd(dppf)Cl₂ (37 mg, 0.005 mmol), and potassium carbonate (28 mg, 0.2 mmol) were stirred in dioxane (1 mL) and water (100 uL) at 80° C. overnight. The material was filtered through Celite© and concentrated. 1 N HCl (500 uL) and THF (1.5 mL) were added to the residue and the solution was stirred for 3 h. The mixture was extracted with DCM and filtered through Celite®. The material was concentrated and purified on a silica gel column eluting with DCM-MeOH to afford (S)-3-(benzylamino)-1-(3,6-dihydro-2H-pyran-4-yl)-4-oxo-4,6,7,8-tetrahydropyrrolo[1,2-a]pyrazine-6-carboxylic acid (4.5 mg, 11% yield).

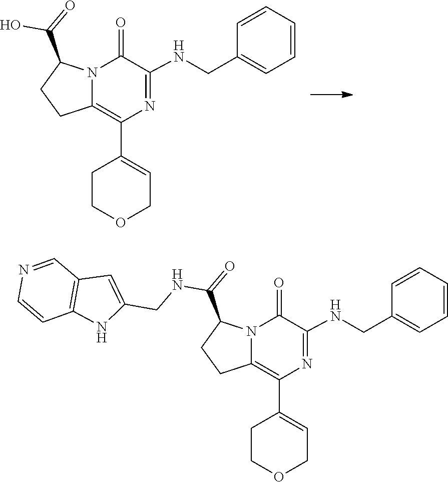 [SMILES: O=C(O)[C@@H]1CCc2c(C3=CCOCC3)nc(NCc3ccccc3)c(=O)n21 ; O=C(NCc1cc2cnccc2[nH]1)[C@@H]1CCc2c(C3=CCOCC3)nc(NCc3ccccc3)c(=O)n21]

Step 3: The above compound was synthesized according to General Amide Coupling Procedure A to afford (S)—N-((1H-pyrrolo[3,2-c]pyridin-2-yl)methyl)-3-(benzylamino)-1-(3,6-dihydro-2H-pyran-4-yl)-4-oxo-4,6,7,8-tetrahydropyrrolo[1,2-a]pyrazine-6-carboxamide (4 mg, 72% yield).

### Example 17

### Preparation of (S)—N-((1H-pyrrolo[3,2-c]pyridin-2-yl)methyl)-3-(benzylamino)-1-cyano-4-oxo-4,6,7,8-tetrahydropyrrolo[1,2-a]pyrazine-6-carboxamide trifluoroacetate

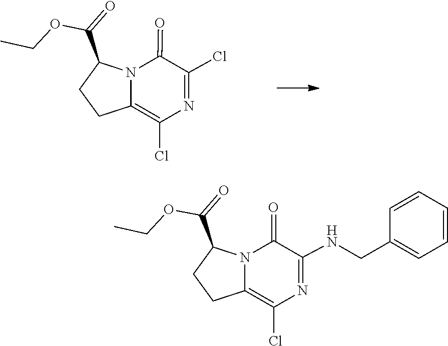 [SMILES: CCOC(=O)[C@@H]1CCc2c(Cl)nc(Cl)c(=O)n21 ; CCOC(=O)[C@@H]1CCc2c(Cl)nc(NCc3ccccc3)c(=O)n21]

Step 1: The above compound was synthesized according to Example 9, step 1.

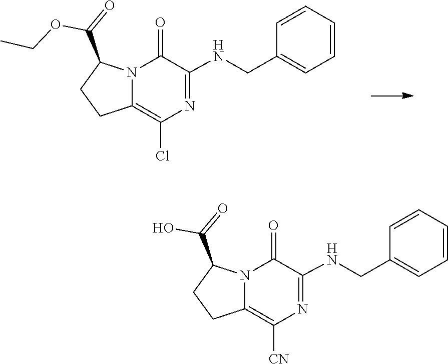 [SMILES: CCOC(=O)[C@@H]1CCc2c(Cl)nc(NCc3ccccc3)c(=O)n21 ; N#Cc1nc(NCc2ccccc2)c(=O)n2c1CC[C@H]2C(=O)O]

Step 2: Ethyl (S)-3-(benzylamino)-1-chloro-4-oxo-4,6,7,8-tetrahydropyrrolo[1,2-a]pyrazine-6-carboxylate (35 mg, 0.1 mmol), zinc cyanide (7 mg, 0.06 mmol), PdTrixiePhos G3 (3.8 mg, 0.005 mmol), and Zn dust (1.3 mg, 0.02 mmol) were stirred in DMA (1 mL) at 90° C. under a blanket of Ar overnight. Additional PdTrixiePhos G3 (3.8 mg, 0.005 mmol) was added and the reaction was stirred for an additional 24 h. The material was filtered through Celite,® concentrated, then chromatographed eluting with DCM-MeOH. The product was isolated as a brown oil (30 mg, 90% yield).

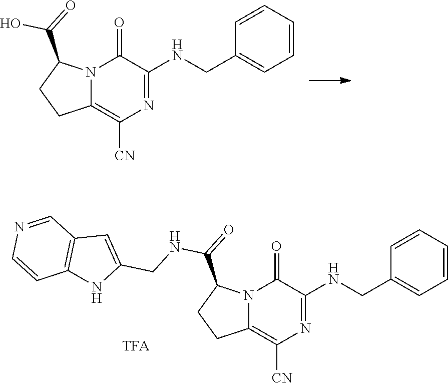 [SMILES: N#Cc1nc(NCc2ccccc2)c(=O)n2c1CC[C@H]2C(=O)O ; N#Cc1nc(NCc2ccccc2)c(=O)n2c1CC[C@H]2C(=O)NCc1cc2cnccc2[nH]1]

Step 3: The above compound was synthesized according to General Amide Coupling Procedure A, except that purification consisted of prep HPLC to afford (S)—N-((1H-pyrrolo[3,2-c]pyridin-2-yl)methyl)-3-(benzylamino)-1-cyano-4-oxo-4,6,7,8-tetrahydropyrrolo[1,2-a]pyrazine-6-carboxamide trifluoroacetate (3.2 mg, 7% yield).

### Example 18

### Preparation of (S)—N-((6-amino-2-methylpyridin-3-yl)methyl)-3′-((3,5-dimethylbenzyl)amino)-4′-oxo-6′,7′-dihydro-4′H-spiro[cyclopropane-1,8′-pyrrolo[1,2-a]pyrazine]-6′-carboxamide trifluoroacetate

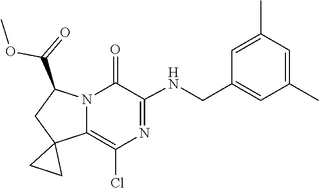

Steps 1-5: The above compound was synthesized with the appropriate starting materials according to Example 2, steps 1-5.

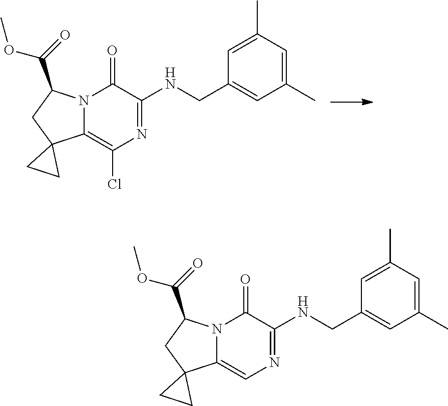

Step 6: The above compound was synthesized according to General Hydrogenolysis Procedure B to afford methyl (S)-3′-((3,5-dimethylbenzyl)amino)-4′-oxo-6′,7′-dihydro-4′H-spiro[cyclopropane-1,8′-pyrrolo[1,2-a]pyrazine]-6′-carboxylate as a clear oil (quantitative yield).

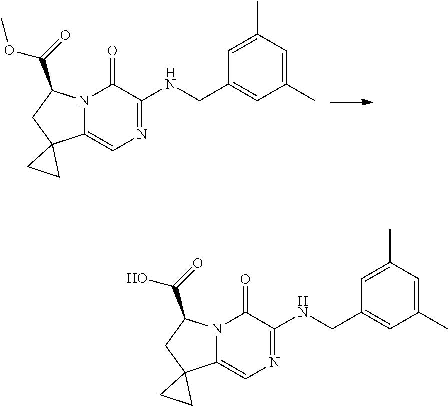

Step 7: Methyl (S)-3′-((3,5-dimethylbenzyl)amino)-4′-oxo-6′,7′-dihydro-4′H-spiro[cyclopropane-1,8′-pyrrolo[1,2-a]pyrazine]-6′-carboxylate (241 mg, 0.683 mmol) and 1 M aq. LiOH (1.37 mL) were added to a solution of THF (6 mL) and H₂O (2 mL), and stirred for 5 h at room temperature. The mixture was concentrated, then acidified with 1N aq. HCl. The white precipitate was collected to afford (S)-3′-((3,5-dimethylbenzyl)amino)-4′-oxo-6′,7′-dihydro-4′H-spiro[cyclopropane-1,8′-pyrrolo[1,2-a]pyrazine]-6′-carboxylic acid (101 mg, 44% yield).

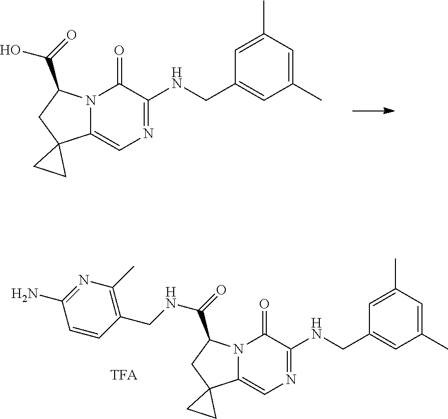 [SMILES: Cc1cc(C)cc(CNc2ncc3n(c2=O)[C@H](C(=O)O)CC32CC2)c1 ; Cc1cc(C)cc(CNc2ncc3n(c2=O)[C@H](C(=O)NCc2ccc(N)nc2C)CC32CC2)c1]

Step 8: The above compound was synthesized according to General Amide Coupling Procedure A, except that prep HPLC was performed to afford (S)—N-((6-amino-2-methylpyridin-3-yl)methyl)-3′-((3,5-dimethylbenzyl)amino)-4′-oxo-6′,7′-dihydro-4′H-spiro[cyclopropane-1,8′-pyrrolo[1,2-a]pyrazine]-6′-carboxamide trifluoroacetate (32 mg, 63% yield).

The following compounds were prepared according to the foregoing procedures with the appropriate amines, except that purification consisted of an amine column eluting with DCM-MeOH, followed by prep HPLC purification:

| | Cmp |
|---|---|
| Compound Name | No. |
| (S)-N-((1H-pyrrolo[3,2-c]pyridin-2-yl)methyl)-3′-((3,5- | 123 |
| dimethylbenzyl)amino)-4′-oxo-6′,7′- | |
| dihydro-4′H-spiro[cyclopropane-1,8′- | |
| pyrrolo[1,2-a]pyrazine]- | |
| 6′-carboxamide trifluoroacetate | |
| (S)-3′-((3,5-dimethylbenzyl)amino)-N-((6-methyl-1H-pyrrolo[3,2- | 124 |
| c]pyridin-2-yl)methyl)-4′-oxo-6′,7′- | |
| dihydro-4′H-spiro[cyclopropane-1,8′- | |
| pyrrolo[1,2-a]pyrazine]-6′-carboxamide trifluoroacetate | |

The following compound was prepared according to the foregoing procedures with the appropriate amine, except that purification consisted of an amine column eluting with DCM-MeOH:

| | Cmp |
|---|---|
| Compound Name | No. |
| (S)-N-((1H-pyrrolo[3,2-c]pyridin- | 125 |
| 2-yl)methyl)-3′-(benzylamino)-4′-oxo- | |
| 6′,7′-dihydro-4′H-spiro[cyclopropane- | |
| 1,8′-pyrrolo[1,2-a]pyrazine]-6′- | |
| carboxamide | |

### Example 19

### Preparation of (S)—N-((5,6-dihydro-4H-thieno[2,3-c]pyrrol-2-yl)methyl)-3′-((3,5-dimethylbenzyl)amino)-4′-oxo-6′,7′-dihydro-4′H-spiro[cyclopropane-1,8′-pyrrolo[1,2-a]pyrazine]-6′-carboxamide

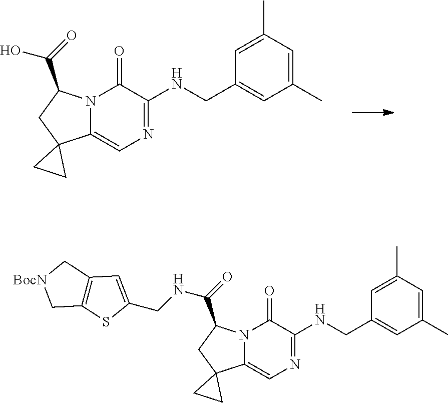 [SMILES: Cc1cc(C)cc(CNc2ncc3n(c2=O)[C@H](C(=O)O)CC32CC2)c1 ; Cc1cc(C)cc(CNc2ncc3n(c2=O)[C@H](C(=O)NCc2cc4c(s2)CN(C(=O)OC(C)(C)C)C4)CC32CC2)c1]

Step 1: The above compound was synthesized according to General Amide Coupling Procedure A to afford tert-butyl (S)-2-((3′-((3,5-dimethylbenzyl)amino)-4′-oxo-6′,7′-dihydro-4′H-spiro[cyclopropane-1,8′-pyrrolo[1,2-a]pyrazine]-6′-carboxamido)methyl)-4,6-dihydro-5H-thieno[2,3-c]pyrrole-5-carboxylate (37 mg, 73% yield).

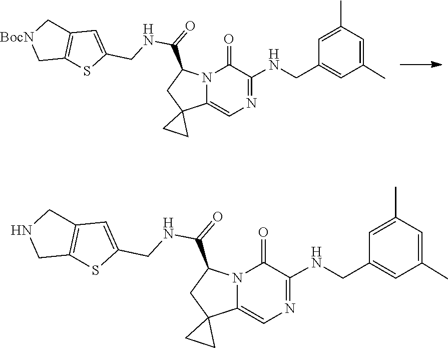 [SMILES: Cc1cc(C)cc(CNc2ncc3n(c2=O)[C@H](C(=O)NCc2cc4c(s2)CN(C(=O)OC(C)(C)C)C4)CC32CC2)c1 ; Cc1cc(C)cc(CNc2ncc3n(c2=O)[C@H](C(=O)NCc2cc4c(s2)CNC4)CC32CC2)c1]

Step 2: The above compound was synthesized according to General Boc Removal Procedure A to afford tert-butyl (S)—N-((5,6-dihydro-4H-thieno[2,3-c]pyrrol-2-yl)methyl)-3′-((3,5-dimethylbenzyl)amino)-4′-oxo-6′,7′-dihydro-4′H-spiro[cyclopropane-1,8′-pyrrolo[1,2-a]pyrazine]-6′-carboxamide (32 mg, 93% yield).

The following compound was prepared according to the foregoing procedures with the appropriate amine, except that the final amide coupling step was purified by prep HPLC:

| | Cmp |
|---|---|
| Compound Name | No. |
| (S)-3′-(benzylamino)-N-((5,6-dihydro-4H- | 127 |
| thieno[2,3-c]pyrrol-2-yl)methyl)- | |
| 4′-oxo-6′,7′-dihydro-4′H-spiro[cyclopropane- | |
| 1,8′-pyrrolo[1,2-a]pyrazine]- | |
| 6′-carboxamide trifluoroacetate | |

### Example 20

### Preparation of (S)—N-((1H-pyrrolo[3,2-c]pyridin-2-yl)methyl)-3′-(benzylamino)-1′-chloro-4′-oxo-6′,7′-dihydro-4′H-spiro[cyclopropane-1,8′-pyrrolo[1,2-a]pyrazine]-6′-carboxamide

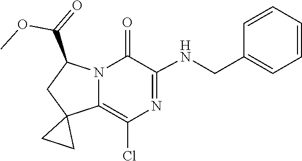 [SMILES: COC(=O)[C@@H]1CC2(CC2)c2c(Cl)nc(NCc3ccccc3)c(=O)n21]

Steps 1-5: The above compound was synthesized with the appropriate starting materials according to Example 2, steps 1-5.

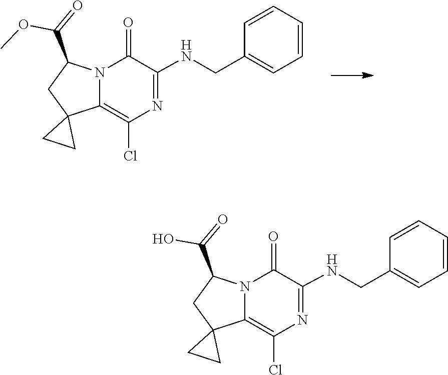 [SMILES: COC(=O)[C@@H]1CC2(CC2)c2c(Cl)nc(NCc3ccccc3)c(=O)n21 ; O=C(O)[C@@H]1CC2(CC2)c2c(Cl)nc(NCc3ccccc3)c(=O)n21]

Step 6: The above compound was synthesized with the appropriate starting materials according to the procedure in Example 18, step 7.

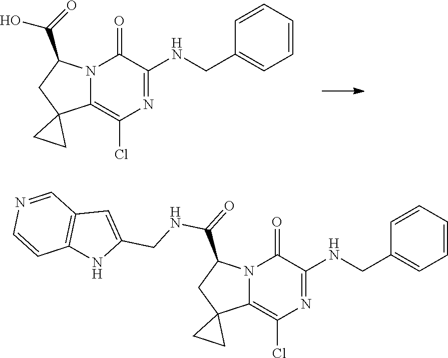 [SMILES: O=C(O)[C@@H]1CC2(CC2)c2c(Cl)nc(NCc3ccccc3)c(=O)n21 ; O=C(NCc1cc2cnccc2[nH]1)[C@@H]1CC2(CC2)c2c(Cl)nc(NCc3ccccc3)c(=O)n21]

Step 7: The above compound was synthesized according to General Amide Coupling Procedure A to afford (S)—N-((1H-pyrrolo[3,2-c]pyridin-2-yl)methyl)-3′-(benzylamino)-1′-chloro-4′-oxo-6′,7′-dihydro-4′H-spiro[cyclopropane-1,8′-pyrrolo[1,2-a]pyrazine]-6′-carboxamide (10 mg, 73% yield).

### Example 21

### Preparation of (6S,8S)-3-((3,5-dimethylbenzyl)amino)-8-ethyl-N-((6-methyl-1H-pyrrolo[3,2-c]pyridin-2-yl)methyl)-4-oxo-4,6,7,8-tetrahydropyrrolo[1,2-a]pyrazine-6-carboxamide

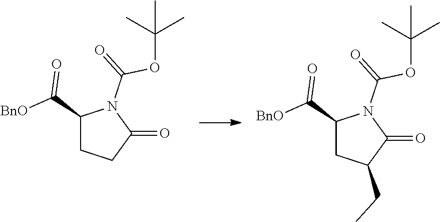 [SMILES: CC(C)(C)OC(=O)N1C(=O)CC[C@H]1C(=O)OCc1ccccc1 ; CC[C@H]1C[C@@H](C(=O)OCc2ccccc2)N(C(=O)OC(C)(C)C)C1=O]

Step 1: To a solution of 2-benzyl 1-(tert-butyl) (S)-5-oxopyrrolidine-1,2-dicarboxylate (9.0 g, 28.2 mmol) at −78° C. was added LiHMDS (9.0 g, 29.6 mmol, 1.0 M in THF) with stirring for 30 min under Ar. Ethyl trifluoromethanesulfonate (5.02 g, 28.2 mmol) was quickly added via syringe and stirring continued for 1 h. The reaction was quenched with sat. aq. NH₄Cl (30 mL) and allowed to warm to room temp. The volatiles were removed in vacuo, then the aqueous mixture was extracted with CH₂Cl₂ (2×50 mL). The organic layers were combined, dried over Na₂SO₄, filtered, and evaporated. Chromatography (20% EtOAc-hexanes) gave 2-benzyl 1-(tert-butyl) (2S,4S)-4-ethyl-5-oxopyrrolidine-1,2-dicarboxylate (4.62 g, 47% yield) as a clear oil.

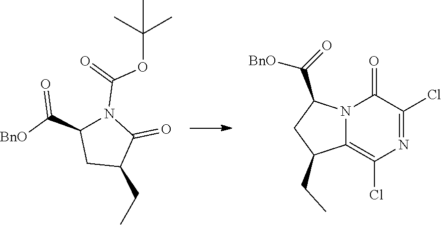 [SMILES: CC[C@H]1C[C@@H](C(=O)OCc2ccccc2)N(C(=O)OC(C)(C)C)C1=O ; CC[C@H]1C[C@@H](C(=O)OCc2ccccc2)n2c1c(Cl)nc(Cl)c2=O]

Steps 2-5: The above compound was synthesized according to Example 2, steps 1-4.

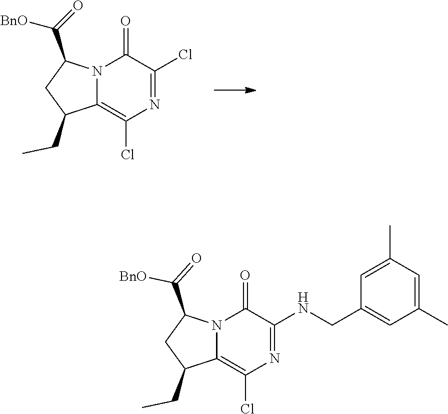 [SMILES: CC[C@H]1C[C@@H](C(=O)OCc2ccccc2)n2c1c(Cl)nc(Cl)c2=O ; CC[C@H]1C[C@@H](C(=O)OCc2ccccc2)n2c1c(Cl)nc(NCc1cc(C)cc(C)c1)c2=O]

Step 6: The above compound was synthesized according to General Chloro Displacement Procedure A to afford benzyl (6S,8S)-1-chloro-3-((3,5-dimethylbenzyl)amino)-8-ethyl-4-oxo-4,6,7,8-tetrahydropyrrolo[1,2-a]pyrazine-6-carboxylate (48 mg, 95% yield).

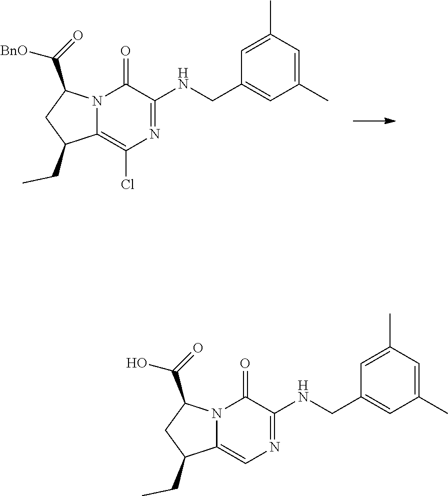

Step 7: The above compound was synthesized according to General Hydrogenolysis Procedure B to afford (6S,8S)-3-((3,5-dimethylbenzyl)amino)-8-ethyl-4-oxo-4,6,7,8-tetrahydropyrrolo[1,2-a]pyrazine-6-carboxylic acid as a clear oil (quantitative yield).

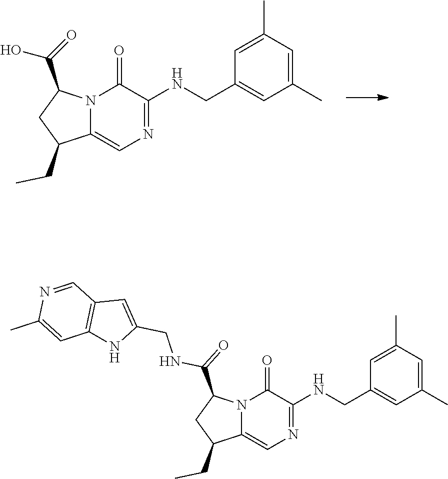

Step 8: The above compound was synthesized according to General Amide Coupling Procedure A to afford (6S,8S)-3-((3,5-dimethylbenzyl)amino)-8-ethyl-N-((6-methyl-1H-pyrrolo[3,2-c]pyridin-2-yl)methyl)-4-oxo-4,6,7,8-tetrahydropyrrolo[1,2-a]pyrazine-6-carboxamide (5.7 mg, 31% yield).

### Example 22

### Preparation of (6S,8S)—N-((1H-pyrrolo[3,2-c]pyridin-2-yl)methyl)-3-(benzylamino)-1-chloro-8-methyl-4-oxo-4,6,7,8-tetrahydropyrrolo[1,2-a]pyrazine-6-carboxamide

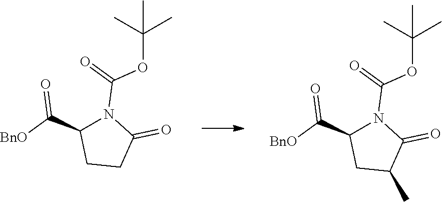 [SMILES: CC(C)(C)OC(=O)N1C(=O)CC[C@H]1C(=O)OCc1ccccc1 ; C[C@H]1C[C@@H](C(=O)OCc2ccccc2)N(C(=O)OC(C)(C)C)C1=O]

Step 1: The above compound was synthesized according to Example 21, step 1 except iodomethane was used as the alkylating reagent. The procedure produced a mixture of bis-methylated and mono-methylated products (see Example 55) which were separated by chromatography (EtOAc/heptane).

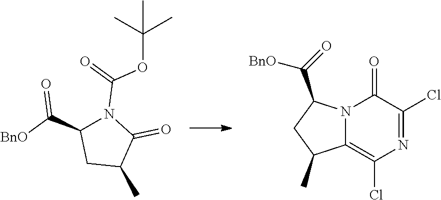 [SMILES: C[C@H]1C[C@@H](C(=O)OCc2ccccc2)N(C(=O)OC(C)(C)C)C1=O ; C[C@H]1C[C@@H](C(=O)OCc2ccccc2)n2c1c(Cl)nc(Cl)c2=O]

Steps 2-5: The above compound was synthesized according to Example 2, steps 1-4.

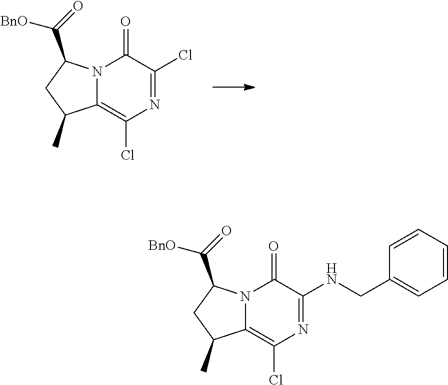 [SMILES: C[C@H]1C[C@@H](C(=O)OCc2ccccc2)n2c1c(Cl)nc(Cl)c2=O ; C[C@H]1C[C@@H](C(=O)OCc2ccccc2)n2c1c(Cl)nc(NCc1ccccc1)c2=O]

Step 6: The above compound was synthesized according to General Chloro Displacement Procedure A to afford benzyl (6S,8S)-3-(benzylamino)-1-chloro-8-methyl-4-oxo-4,6,7,8-tetrahydropyrrolo[1,2-a]pyrazine-6-carboxylate as an oil (110 mg, 74% yield).

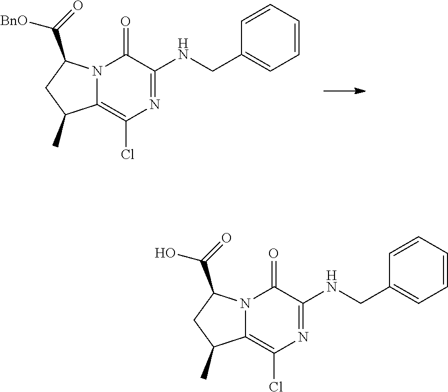 [SMILES: C[C@H]1C[C@@H](C(=O)OCc2ccccc2)n2c1c(Cl)nc(NCc1ccccc1)c2=O ; C[C@H]1C[C@@H](C(=O)O)n2c1c(Cl)nc(NCc1ccccc1)c2=O]

Step 7: The above compound was synthesized according to General Hydrogenolysis Procedure A to afford (6S,8S)-3-(benzylamino)-1-chloro-8-methyl-4-oxo-4,6,7,8-tetrahydropyrrolo[1,2-a]pyrazine-6-carboxylic acid as an off-white solid (quantitative yield).

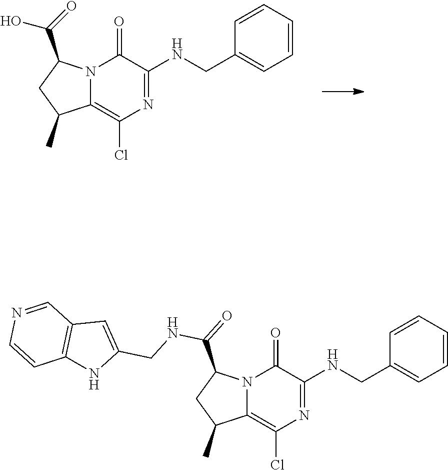 [SMILES: C[C@H]1C[C@@H](C(=O)O)n2c1c(Cl)nc(NCc1ccccc1)c2=O ; C[C@H]1C[C@@H](C(=O)NCc2cc3cnccc3[nH]2)n2c1c(Cl)nc(NCc1ccccc1)c2=O]

Step 8: The above compound was synthesized according to General Amide Coupling Procedure B to afford (6S,8S)—N-((1H-pyrrolo[3,2-c]pyridin-2-yl)methyl)-3-(benzylamino)-1-chloro-8-methyl-4-oxo-4,6,7,8-tetrahydropyrrolo[1,2-a]pyrazine-6-carboxamide as a pale-yellow solid (20 mg, 48% yield).

The following compounds were prepared according to the foregoing procedures using the appropriate amines:

| | Cmp |
|---|---|
| Compound Name | No. |
| (S)-N-((6-amino-2-methylpyridin-3-yl)methyl)- | 131 |
| 3-(benzylamino)-1-chloro- | |
| 4-oxo-4,6,7,8-tetrahydropyrrolo[1,2- | |
| a]pyrazine-6-carboxamide | |
| (6S,8S)-N-((1H-pyrrolo[3,2-c]pyridin- | 132 |
| 2-yl)methyl)-1-chloro-3-((3,5- | |
| dimethylbenzyl)amino)-8-methyl-4-oxo- | |
| 4,6,7,8-tetrahydropyrrolo[1,2- | |
| a]pyrazine-6-carboxamide | |
| (S)-N-((1H-pyrrolo[3,2-c]pyridin- | 133 |
| 2-yl)methyl)-1-chloro-3-((3- | |
| cyclopropylbenzyl)amino)-4-oxo- | |
| 4,6,7,8-tetrahydropyrrolo[1,2-a]pyrazine- | |
| 6-carboxamide | |
| (6S,8S)-N-((6-amino-2-methylpyridin- | 134 |
| 3-yl)methyl)-3-(benzylamino)-1- | |
| chloro-8-ethyl-4-oxo-4,6,7,8- | |
| tetrahydropyrrolo[1,2-a]pyrazine-6- | |
| carboxamide | |
| (R)-N-((6-amino-2-methylpyridin-3-yl)methyl)- | 135 |
| 3-(benzylamino)-1-chloro- | |
| 4-oxo-4,6,7,8-tetrahydropyrrolo[1,2- | |
| a]pyrazine-6-carboxamide | |

The following compound was prepared according to the foregoing procedures, except conc HCl (3 drops) was used in step 2, and DIEA (2 eq) was used in step 3 with the appropriate amine:

| | Cmp |
|---|---|
| Compound Name | No. |
| (S)-N-((6-amino-2-methylpyridin-3- | 136 |
| yl)methyl)-1-chloro-4-oxo-3-((pyridin- | |
| 3-ylmethyl)amino)-4,6,7,8-tetrahydropyrrolo[1,2- | |
| a]pyrazine-6-carboxamide | |

### Example 23

### Preparation of (6S,8S)-8-benzyl-3-((3-methoxy-5-methylbenzyl)amino)-N-((6-methyl-1H-pyrrolo[3,2-c]pyridin-2-yl)methyl)-4-oxo-4,6,7,8-tetrahydropyrrolo[1,2-a]pyrazine-6-carboxamide

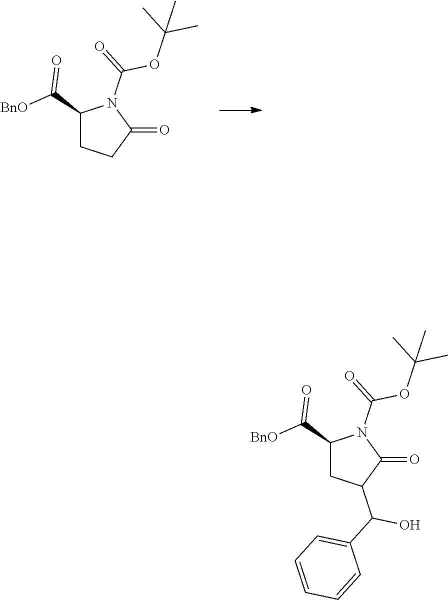 [SMILES: CC(C)(C)OC(=O)N1C(=O)CC[C@H]1C(=O)OCc1ccccc1 ; CC(C)(C)OC(=O)N1C(=O)C(C(O)c2ccccc2)C[C@H]1C(=O)OCc1ccccc1]

Step 1: To a solution of 2-benzyl 1-(tert-butyl) (S)-5-oxopyrrolidine-1,2-dicarboxylate (10.0 g, 31.3 mmol) at −78° C. was added LiHMDS (5.76 g, 34.4 mmol, 1.0 M in THF) with stirring for 30 min under Ar. Benzaldehyde (3.66 g, 34.4 mmol) was added via syringe and stirring continued for 1 h. The reaction was quenched with sat. aq. NH₄Cl (50 mL) and allowed to warm to room temp. The volatiles were removed in vacuo, then the aqueous mixture was extracted with CH₂Cl₂ (2×50 mL). The organic layers were combined and dried over Na₂SO₄ to give crude 2-benzyl 1-(tert-butyl) (2S)-4-(hydroxy(phenyl)methyl)-5-oxopyrrolidine-1,2-dicarboxylate (12.8 g, 96% yield), which was used as is in the next step.

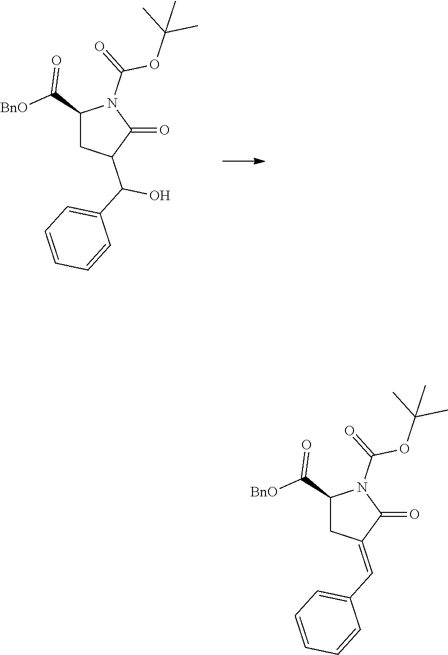 [SMILES: CC(C)(C)OC(=O)N1C(=O)C(C(O)c2ccccc2)C[C@H]1C(=O)OCc1ccccc1 ; CC(C)(C)OC(=O)N1C(=O)/C(=C/c2ccccc2)C[C@H]1C(=O)OCc1ccccc1]

Step 2: To a solution of 2-benzyl 1-(tert-butyl) (2S)-4-(hydroxy(phenyl)methyl)-5-oxopyrrolidine-1,2-dicarboxylate (12.8 g, 30.1 mmol crude) in DCM (50 mL) was added Et₃N (4.57 g, 45.1 mmol) and methanesulfonyl chloride (5.17 g, 45.1 mmol) with stirring at room temp. After 72 h, the solution was extracted with 10% KHSO₄ (50 mL) and the organic layer was dried over Na₂SO₄ and evaporated. Column chromatography (25% EtOAc-hexanes) gave 2-benzyl 1-(tert-butyl) (S,E)-4-benzylidene-5-oxopyrrolidine-1,2-dicarboxylate (5.2 g, 42% yield) as a thick oil.

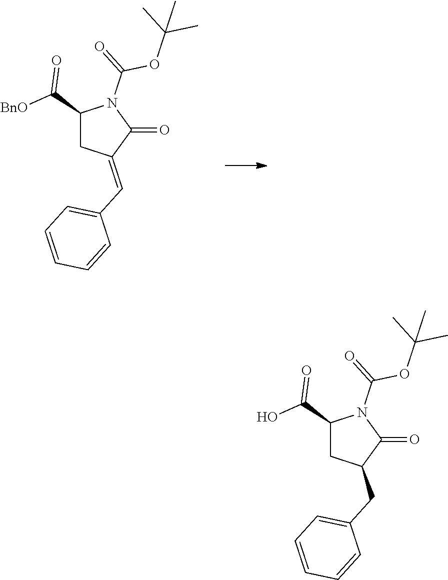 [SMILES: CC(C)(C)OC(=O)N1C(=O)/C(=C/c2ccccc2)C[C@H]1C(=O)OCc1ccccc1 ; CC(C)(C)OC(=O)N1C(=O)[C@@H](Cc2ccccc2)C[C@H]1C(=O)O]

Step 3: To a degassed solution of 2-benzyl 1-(tert-butyl) (S,E)-4-benzylidene-5-oxopyrrolidine-1,2-dicarboxylate (5.0 g, 12 mmol) in MeOH (50 mL) was added 10% Pd/C (100 mg). The mixture was stirred under 1 atm H₂. After 6 h, the catalyst was removed by filtration, followed by evaporation to dryness, affording (2S,4S)-4-benzyl-1-(tert-butoxycarbonyl)-5-oxopyrrolidine-2-carboxylic acid (3.81 g, 97% yield).

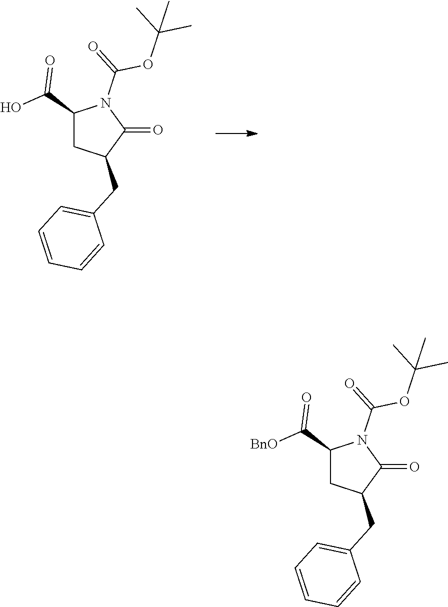 [SMILES: CC(C)(C)OC(=O)N1C(=O)[C@@H](Cc2ccccc2)C[C@H]1C(=O)O ; CC(C)(C)OC(=O)N1C(=O)[C@@H](Cc2ccccc2)C[C@H]1C(=O)OCc1ccccc1]

Step 4: To a solution of (2S,4S)-4-benzyl-1-(tert-butoxycarbonyl)-5-oxopyrrolidine-2-carboxylic acid 3.8 g, 12 mmol) in DMF (20 mL) was added cesium carbonate (4.7 g, 14 mmol) and benzyl bromide (2.4 g, 14 mmol) with stirring at room temp overnight. The solution was evaporated to dryness and the residue extracted with 10% KHSO₄ (30 mL) and EtOAc (2×50 mL). The combined organic layers were dried over Na₂SO₄, filtered, and evaporated. Column chromotography (20% EtOAc-hexanes) gave 2-benzyl 1-(tert-butyl) (2S,4S)-4-benzyl-5-oxopyrrolidine-1,2-dicarboxylate (3.5 g, 72% yield) as a thick oil.

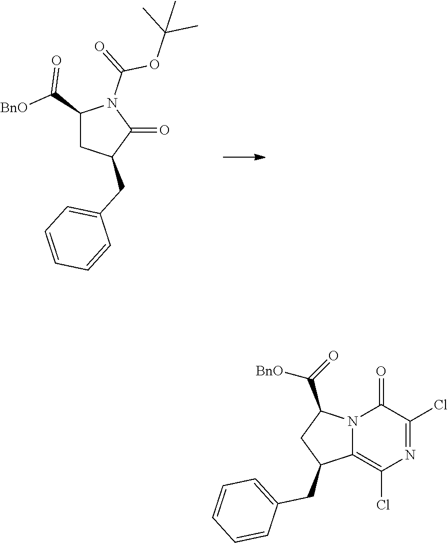 [SMILES: CC(C)(C)OC(=O)N1C(=O)[C@@H](Cc2ccccc2)C[C@H]1C(=O)OCc1ccccc1 ; O=C(OCc1ccccc1)[C@@H]1C[C@H](Cc2ccccc2)c2c(Cl)nc(Cl)c(=O)n21]

Steps 5-8: The above compound was synthesized according to Example 2, steps 1-4.

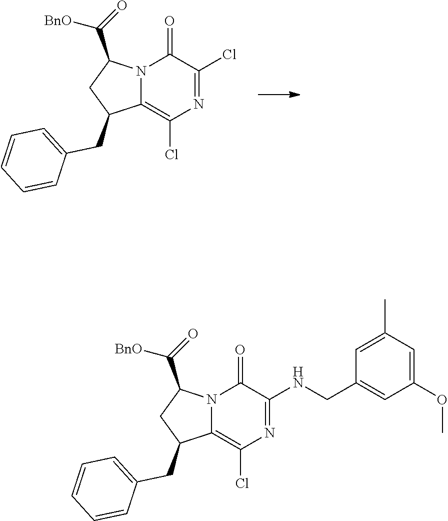 [SMILES: O=C(OCc1ccccc1)[C@@H]1C[C@H](Cc2ccccc2)c2c(Cl)nc(Cl)c(=O)n21 ; COc1cc(C)cc(CNc2nc(Cl)c3n(c2=O)[C@H](C(=O)OCc2ccccc2)C[C@@H]3Cc2ccccc2)c1]

Step 9: The above compound was synthesized according to general Chloro Displacement Procedure A to afford benzyl (6S,8S)-8-benzyl-1-chloro-3-((3-methoxy-5-methylbenzyl)amino)-4-oxo-4,6,7,8-tetrahydropyrrolo[1,2-a]pyrazine-6-carboxylate (202 mg, 66% yield).

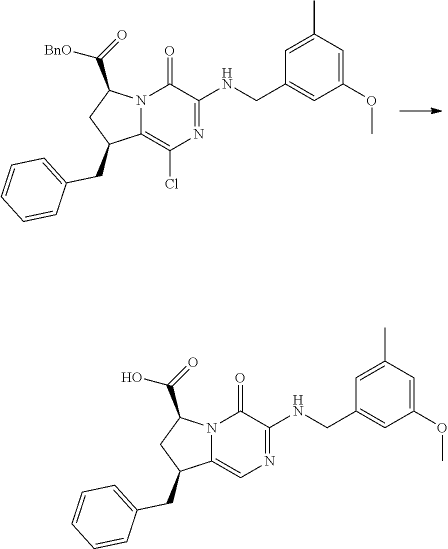 [SMILES: COc1cc(C)cc(CNc2nc(Cl)c3n(c2=O)[C@H](C(=O)OCc2ccccc2)C[C@@H]3Cc2ccccc2)c1 ; COc1cc(C)cc(CNc2ncc3n(c2=O)[C@H](C(=O)O)C[C@@H]3Cc2ccccc2)c1]

Step 10: The above compound was synthesized according to General Hydrogenolysis Procedure B to afford (6S,8S)-8-benzyl-3-((3-methoxy-5-methylbenzyl)amino)-4-oxo-4,6,7,8-tetrahydropyrrolo[1,2-a]pyrazine-6-carboxylic acid) as an oil (quantitative yield).

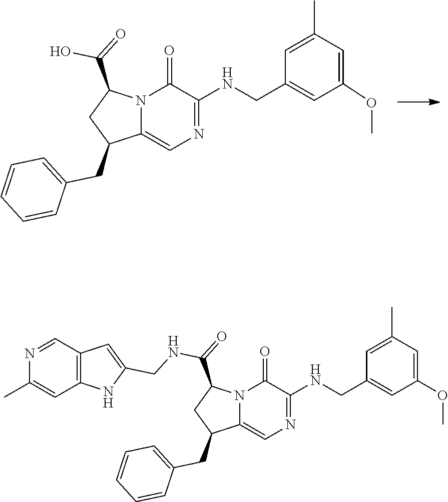

Step 11: The above compound was synthesized according to General Amide Coupling Procedure B to afford (6S,8S)-8-benzyl-3-((3-methoxy-5-methylbenzyl)amino)-N-((6-methyl-1H-pyrrolo[3,2-c]pyridin-2-yl)methyl)-4-oxo-4,6,7,8-tetrahydropyrrolo[1,2-a]pyrazine-6-carboxamide as a pale yellow solid (32 mg, 30% yield).

The following compounds were prepared according to the foregoing procedures using the appropriate amines:

| | Cmp |
|---|---|
| Compound Name | No. |
| (6S,8S)-N-((6-amino-2-methylpyridin- | 138 |
| 3-yl)methyl)-8-benzyl-3-((3- | |
| methoxy-5-methylbenzyl)amino)-4- | |
| oxo-4,6,7,8-tetrahydropyrrolo[1,2- | |
| a]pyrazine-6-carboxamide | |
| (6S,8S)-8-benzyl-3-(cyclobutylamino)- | 139 |
| N-((6-methyl-1H-pyrrolo[3,2- | |
| c]pyridin-2-yl)methyl)-4-oxo-4,6,7,8- | |
| tetrahydropyrrolo[1,2-a]pyrazine-6- | |
| carboxamide | |
| (6S,8S)-N-((6-amino-2-methylpyridin- | 140 |
| 3-yl)methyl)-8-benzyl-3- | |
| (cyclobutylamino)-4-oxo-4,6,7,8- | |
| tetrahydropyrrolo[1,2- | |
| a]pyrazine-6-carboxamide | |
| (6S,8S)-N-((1H-pyrrolo[3,2-c]pyridin- | 141 |
| 2-yl)methyl)-8-benzyl-3-((3- | |
| methoxy-5-methylbenzyl)amino)-4-oxo- | |
| 4,6,7,8-tetrahydropyrrolo[1,2- | |
| a]pyrazine-6-carboxamide | |
| (6S,8S)-N-((1H-pyrrolo[3,2-c]pyridin- | 142 |
| 2-yl)methyl)-8-benzyl-3- | |
| (cyclobutylamino)-4-oxo-4,6,7,8- | |
| tetrahydropyrrolo[1,2- | |
| a]pyrazine-6-carboxamide | |

### Example 24

### Preparation of (S)—N-((1H-pyrrolo[3,2-c]pyridin-2-yl)methyl)-3-(([1,1′-biphenyl]-3-ylmethyl)amino)-1-chloro-4-oxo-4,6,7,8-tetrahydropyrrolo[1,2-a]pyrazine-6-carboxamide

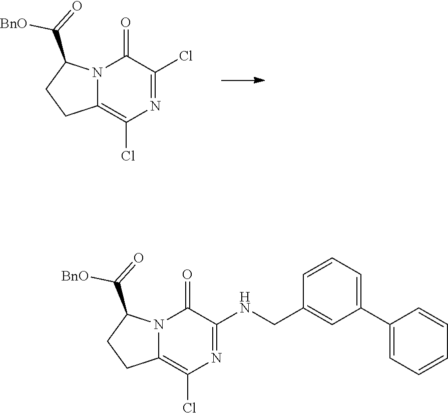 [SMILES: O=C(OCc1ccccc1)[C@@H]1CCc2c(Cl)nc(Cl)c(=O)n21 ; O=C(OCc1ccccc1)[C@@H]1CCc2c(Cl)nc(NCc3cccc(-c4ccccc4)c3)c(=O)n21]

Step 1: The above compound was synthesized according to general Chloro Displacement Procedure A to afford benzyl (S)-3-(([1,1′-biphenyl]-3-ylmethyl)amino)-1-chloro-4-oxo-4,6,7,8-tetrahydropyrrolo[1,2-a]pyrazine-6-carboxylate (169 mg, 79% yield).

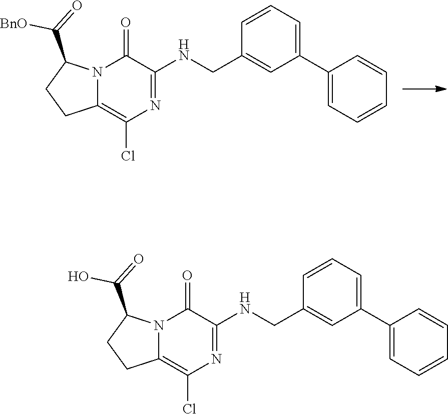 [SMILES: O=C(OCc1ccccc1)[C@@H]1CCc2c(Cl)nc(NCc3cccc(-c4ccccc4)c3)c(=O)n21 ; O=C(O)[C@@H]1CCc2c(Cl)nc(NCc3cccc(-c4ccccc4)c3)c(=O)n21]

Step 2: To a solution of benzyl (S)-3-(([1,1′-biphenyl]-3-ylmethyl)amino)-1-chloro-4-oxo-4,6,7,8-tetrahydropyrrolo[1,2-a]pyrazine-6-carboxylate (169 mg, 348 μmol) in THF (5 mL) and MeOH (2.5 mL) was added LiOH (41.6 mg, 1.74 mmol) in H₂O (2.5 mL) with stirring for 4 h at room temp. The solution was evaporated to dryness, and H₂O (10 mL) was added with stirring. The pH was adjusted to approx 2 with the dropwise addition of conc HCl. The product was collected by filtration, washed with H₂O (2×2 mL), and dried giving benzyl (S)-3-(([1,1′-biphenyl]-3-ylmethyl)amino)-1-chloro-4-oxo-4,6,7,8-tetrahydropyrrolo[1,2-a]pyrazine-6-carboxylate (104 mg, 76% yield) as a pale yellow solid.

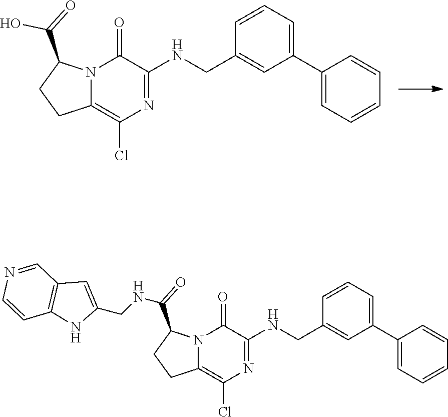 [SMILES: O=C(O)[C@@H]1CCc2c(Cl)nc(NCc3cccc(-c4ccccc4)c3)c(=O)n21 ; O=C(NCc1cc2cnccc2[nH]1)[C@@H]1CCc2c(Cl)nc(NCc3cccc(-c4ccccc4)c3)c(=O)n21]

Step 3: The above compound was synthesized according to General Amide Coupling Procedure B to afford (S)—N-((1H-pyrrolo[3,2-c]pyridin-2-yl)methyl)-3-(([1,1′-biphenyl]-3-ylmethyl)amino)-1-chloro-4-oxo-4,6,7,8-tetrahydropyrrolo[1,2-a]pyrazine-6-carboxamide as a pale-yellow solid (30 mg, 57% yield).

The following compounds were prepared according to the foregoing procedures using the appropriate amines:

| | Cmp |
|---|---|
| Compound Name | No. |
| (S)-N-((1H-pyrrolo[3,2-c]pyridin- | 144 |
| 2-yl)methyl)-1-chloro-3-((3- | |
| cyanobenzyl)amino)-4-oxo-4,6,7,8- | |
| tetrahydropyrrolo[1,2- | |
| a]pyrazine-6-carboxamide | |
| (6S)-N-((1H-pyrrolo[3,2-c]pyridin- | 145 |
| 2-yl)methyl)-1-chloro-4-oxo-3- | |
| (((1,2,3,4-tetrahydronaphthalen- | |
| 2-yl)methyl)amino)-4,6,7,8- | |
| tetrahydropyrrolo[1,2-a]pyrazine-6-carboxamide | |
| (S)-N-((6-amino-2-methylpyridin-3- | 146 |
| yl)methyl)-1-chloro-3-((oxetan-3- | |
| ylmethyl)amino)-4-oxo-4,6,7,8-tetrahydropyrrolo[1,2- | |
| a]pyrazine-6-carboxamide | |

### Example 25

### Preparation of (S)—N-((1H-pyrrolo[3,2-c]pyridin-2-yl)methyl)-3-(([1,1′-biphenyl]-3-ylmethyl)amino)-4-oxo-4,6,7,8-tetrahydropyrrolo[1,2-a]pyrazine-6-carboxamide

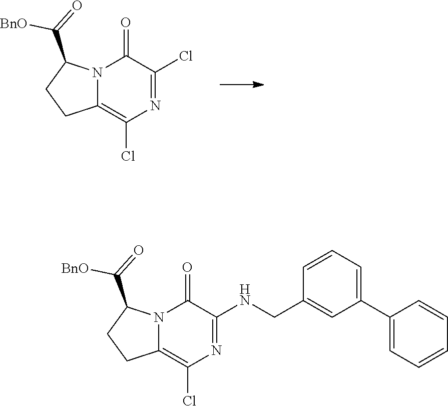 [SMILES: O=C(OCc1ccccc1)[C@@H]1CCc2c(Cl)nc(Cl)c(=O)n21 ; O=C(OCc1ccccc1)[C@@H]1CCc2c(Cl)nc(NCc3cccc(-c4ccccc4)c3)c(=O)n21]

Step 1: The above compound was synthesized according to General Chloro Displacement Procedure A to afford benzyl (S)-3-(([1,1′-biphenyl]-3-ylmethyl)amino)-1-chloro-4-oxo-4,6,7,8-tetrahydropyrrolo[1,2-a]pyrazine-6-carboxylate (90 mg, 90% yield) as a yellow solid.

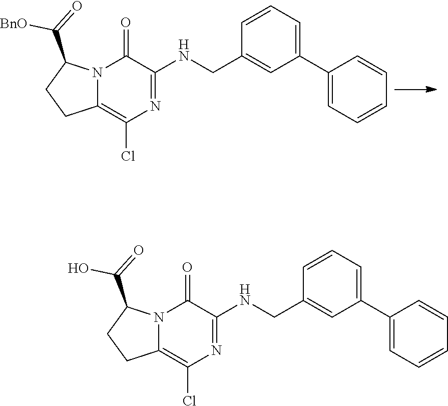 [SMILES: O=C(OCc1ccccc1)[C@@H]1CCc2c(Cl)nc(NCc3cccc(-c4ccccc4)c3)c(=O)n21 ; O=C(O)[C@@H]1CCc2c(Cl)nc(NCc3cccc(-c4ccccc4)c3)c(=O)n21]

Step 2: benzyl (S)-3-(([1,1′-biphenyl]-3-ylmethyl)amino)-1-chloro-4-oxo-4,6,7,8-tetrahydropyrrolo[1,2-a]pyrazine-6-carboxylate was hydrolyzed according to General Saponification Procedure A to give (S)-3-(([1,1′-biphenyl]-3-ylmethyl)amino)-1-chloro-4-oxo-4,6,7,8-tetrahydropyrrolo[1,2-a]pyrazine-6-carboxylic acid as a pale yellow solid (104 mg, 76% yield).

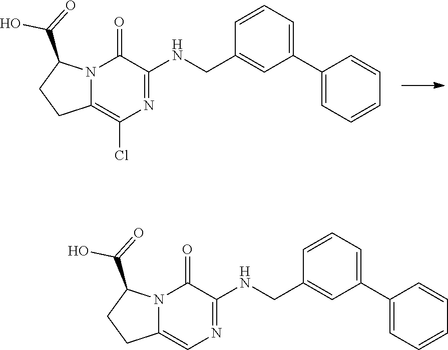 [SMILES: O=C(O)[C@@H]1CCc2c(Cl)nc(NCc3cccc(-c4ccccc4)c3)c(=O)n21 ; O=C(O)[C@@H]1CCc2cnc(NCc3cccc(-c4ccccc4)c3)c(=O)n21]

Step 3: To a solution of (S)-3-(([1,1′-biphenyl]-3-ylmethyl)amino)-1-chloro-4-oxo-4,6,7,8-tetrahydropyrrolo[1,2-a]pyrazine-6-carboxylic acid (64 mg, 0.16 mmol) in MeOH (5 ml) in a stainless steel hydrogenation apparatus, was added DIEA (52 mg, 0.4 mmol) and 10% Pd/C (20 mg). The solution was placed under H₂ (120 psi) overnight. The catalyst was removed by filtration followed by evaporation giving (S)-3-(([1,1′-biphenyl]-3-ylmethyl)amino)-4-oxo-4,6,7,8-tetrahydropyrrolo[1,2-a]pyrazine-6-carboxylic acid as an oil (quantitative yield).

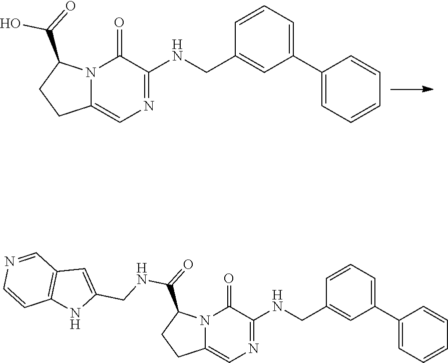 [SMILES: O=C(O)[C@@H]1CCc2cnc(NCc3cccc(-c4ccccc4)c3)c(=O)n21 ; O=C(NCc1cc2cnccc2[nH]1)[C@@H]1CCc2cnc(NCc3cccc(-c4ccccc4)c3)c(=O)n21]

Step 4: The above compound was synthesized according to General Amide Coupling Procedure B to afford (S)—N-((1H-pyrrolo[3,2-c]pyridin-2-yl)methyl)-3-(([1,1′-biphenyl]-3-ylmethyl)amino)-4-oxo-4,6,7,8-tetrahydropyrrolo[1,2-a]pyrazine-6-carboxamide as a pale-yellow solid (53 mg, 67% yield).

The following compound was prepared according to the foregoing procedures using the appropriate amine:

| | Cmp |
|---|---|
| Compound Name | No. |
| (6S)-N-((1H-pyrrolo[3,2-c]pyridin-2- | 148 |
| yl)methyl)-4-oxo-3-(((1,2,3,4- | |
| tetrahydronaphthalen-2-yl)methyl)amino)- | |
| 4,6,7,8-tetrahydropyrrolo[1,2- | |
| alpyrazine-6-carboxamide | |

### Example 26

### Preparation of N-((6-amino-2-methylpyridin-3-yl)methyl)-1-chloro-4-oxo-3-((pyrazin-2-ylmethyl)amino)-4,6,7,8-tetrahydropyrrolo[1,2-a]pyrazine-6-carboxamide

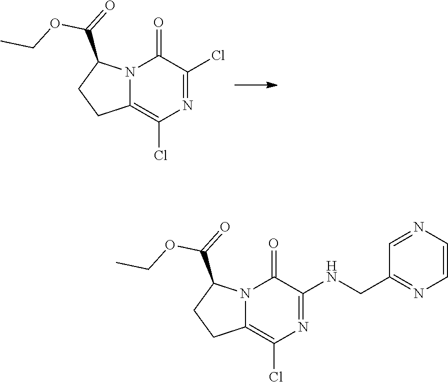 [SMILES: CCOC(=O)[C@@H]1CCc2c(Cl)nc(Cl)c(=O)n21 ; CCOC(=O)[C@@H]1CCc2c(Cl)nc(NCc3cnccn3)c(=O)n21]

Step 1: The above compound was synthesized according to General Chloro Displacement Procedure A to afford 1-chloro-4-oxo-3-((pyrazin-2-ylmethyl)amino)-4,6,7,8-tetrahydropyrrolo[1,2-a]pyrazine-6-carboxylic acid hydrochloride (133 mg, 100% yield).

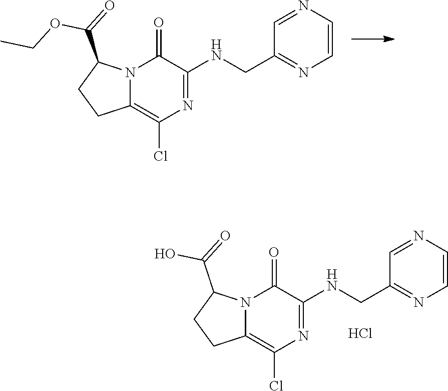 [SMILES: CCOC(=O)[C@@H]1CCc2c(Cl)nc(NCc3cnccn3)c(=O)n21 ; O=C(O)C1CCc2c(Cl)nc(NCc3cnccn3)c(=O)n21 ; Cl]

Step 2: The above compound was synthesized according to General Saponification Procedure A to afford ethyl (S)-1-chloro-4-oxo-3-((pyrazin-2-ylmethyl)amino)-4,6,7,8-tetrahydropyrrolo[1,2-a]pyrazine-6-carboxylate (132 mg, 61%) as an oil.

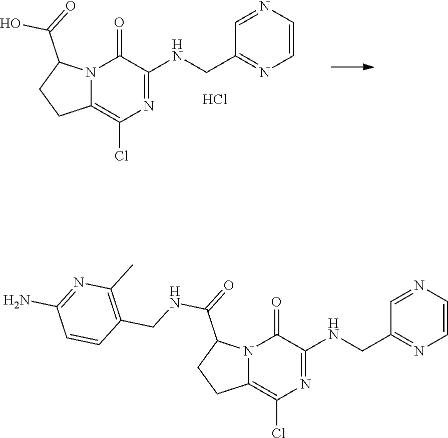 [SMILES: O=C(O)C1CCc2c(Cl)nc(NCc3cnccn3)c(=O)n21 ; Cl ; Cc1nc(N)ccc1CNC(=O)C1CCc2c(Cl)nc(NCc3cnccn3)c(=O)n21]

Step 3: The above compound was synthesized according to General Amide Coupling Procedure B, with the addition of DIEA (2 eq), to afford N-((6-amino-2-methylpyridin-3-yl)methyl)-1-chloro-4-oxo-3-((pyrazin-2-ylmethyl)amino)-4,6,7,8-tetrahydropyrrolo[1,2-a]pyrazine-6-carboxamide as a yellow solid (26 mg, 32% yield).

The following compound was prepared according to the foregoing procedures using the appropriate amine:

| | Cmp |
|---|---|
| Compound Name | No. |
| N-((6-amino-2-methylpyridin-3-yl)methyl)-1-chloro-4-oxo-3- | 150 |
| ((pyrimidin-5-ylmethyl)amino)-4,6,7,8-tetrahydropyrrolo[1,2- | |
| a]pyrazine-6-carboxamide | |

### Example 27

### Preparation of (6S,8S)—N-((1H-pyrrolo[3,2-c]pyridin-2-yl)methyl)-3-(benzylamino)-8-ethyl-4-oxo-4,6,7,8-tetrahydropyrrolo[1,2-a]pyrazine-6-carboxamide

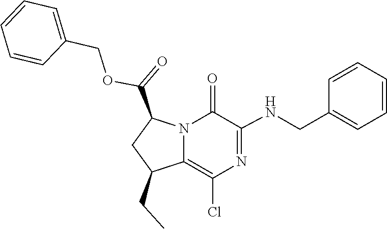 [SMILES: CC[C@H]1C[C@@H](C(=O)OCc2ccccc2)n2c1c(Cl)nc(NCc1ccccc1)c2=O]

Steps 1-6: The above compound was synthesized with the appropriate starting materials according to Example 21, steps 1-6.

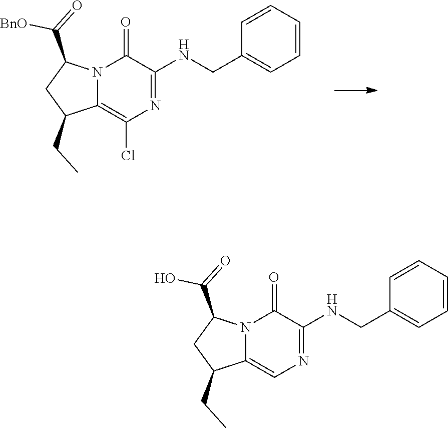 [SMILES: CC[C@H]1C[C@@H](C(=O)OCc2ccccc2)n2c1c(Cl)nc(NCc1ccccc1)c2=O ; CC[C@H]1C[C@@H](C(=O)O)n2c1cnc(NCc1ccccc1)c2=O]

Step 7: To a solution of (6S,8S)-1-chloro-3-((3,5-dimethylbenzyl)amino)-8-ethyl-4-oxo-4,6,7,8-tetrahydropyrrolo[1,2-a]pyrazine-6-carboxylic acid (158 mg, 0.420 mmol) in MeOH (5 mL) in a stainless-steel hydrogenation apparatus, was added DIEA (109 mg, 0.84 mmol) and 10% Pd/C (40 mg). The solution was placed under H₂ (120 psi) with stirring for 72 h. The catalyst was removed by filtration, followed by evaporation to dryness giving (6S,8S)-3-((3,5-dimethylbenzyl)amino)-8-ethyl-4-oxo-4,6,7,8-tetrahydropyrrolo[1,2-a]pyrazine-6-carboxylic acid (143 mg, quantitative yield) as an oil.

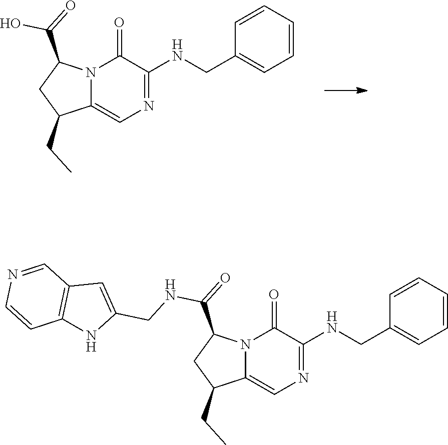 [SMILES: CC[C@H]1C[C@@H](C(=O)O)n2c1cnc(NCc1ccccc1)c2=O ; CC[C@H]1C[C@@H](C(=O)NCc2cc3cnccc3[nH]2)n2c1cnc(NCc1ccccc1)c2=O]

Step 8: The above compound was synthesized according to General Amide Coupling Procedure B to afford (6S,8S)—N-((1H-pyrrolo[3,2-c]pyridin-2-yl)methyl)-3-(benzylamino)-8-ethyl-4-oxo-4,6,7,8-tetrahydropyrrolo[1,2-a]pyrazine-6-carboxamide as an off white solid (47 mg, 30% yield).

The following compounds were prepared according to the foregoing procedures using the appropriate amines:

| | Cmp |
|---|---|
| Compound Name | No. |
| (6S,8S)-N-((1H-pyrrolo[3,2-c]pyridin-2-yl)methyl)-3- | 152 |
| (benzylamino)-8-methyl-4-oxo-4,6,7,8-tetrahydropyrrolo[1,2- | |
| a]pyrazine-6-carboxamide | |
| (6S,8S)-N-((1H-pyrrolo[3,2-c]pyridin-2-yl)methyl)-3-((3,5- | 153 |
| dimethylbenzyl)amino)-8-methyl-4-oxo-4,6,7,8- | |
| tetrahydropyrrolo[1,2-a]pyrazine-6-carboxamide | |
| (6S,8S)-N-((6-amino-2-methylpyridin-3-yl)methyl)-3- | 154 |
| (benzylamino)-8-ethyl-4-oxo-4,6,7,8-tetrahydropyrrolo[1,2- | |
| a]pyrazine-6-carboxamide | |
| (6S,8S)-N-((1H-pyrrolo[3,2-c]pyridin-2-yl)methyl)-3-((3,5- | 155 |
| dimethylbenzyl)amino)-8-ethyl-4-oxo-4,6,7,8- | |
| tetrahydropyrrolo[1,2-a]pyrazine-6-carboxamide | |
| (6S,8S)-N-((6-amino-2-methylpyridin-3-yl)methyl)-3-((3,5- | 156 |
| dimethylbenzyl)amino)-8-ethyl-4-oxo-4,6,7,8- | |
| tetrahydropyrrolo[1,2-a]pyrazine-6-carboxamide | |
| (6S,8S)-N-((6-aminopyridin-3-yl)methyl)-3-((3,5- | 157 |
| dimethylbenzyl)amino)-8-ethyl-4-oxo-4,6,7,8- | |
| tetrahydropyrrolo[1,2-a]pyrazine-6-carboxamide | |

### Example 28

### Preparation of (6S,8S)-3-(benzylamino)-1-chloro-N-((5,6-dihydro-4H-thieno[2,3-c]pyrrol-2-yl)methyl)-8-ethyl-4-oxo-4,6,7,8-tetrahydropyrrolo[1,2-a]pyrazine-6-carboxamide hydrochloride

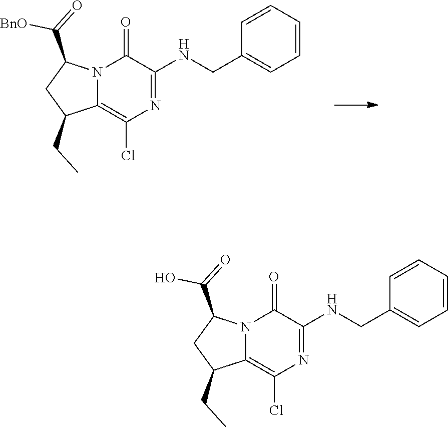 [SMILES: CC[C@H]1C[C@@H](C(=O)OCc2ccccc2)n2c1c(Cl)nc(NCc1ccccc1)c2=O ; CC[C@H]1C[C@@H](C(=O)O)n2c1c(Cl)nc(NCc1ccccc1)c2=O]

Step 1: To a solution of benzyl (6S,8S)-1-chloro-3-((3,5-dimethylbenzyl)amino)-8-ethyl-4-oxo-4,6,7,8-tetrahydropyrrolo[1,2-a]pyrazine-6-carboxylate (249 mg, 534 μmol) in THF (5 mL) and MeOH (2.5 mL) was added LiOH (70 mg) in H₂O (2.5 mL) with stirring for 4 h at room temp. The solution was evaporated to dryness and H₂O (10 mL) was added with stirring. The pH was adjusted to approximately 2 with the dropwise addition of cone HCl, followed by extraction with CH₂Cl₂ (2×20 mL). The organic layers were combined and dried over Na₂SO₄ to give crude (6S,8S)-1-chloro-3-((3,5-dimethylbenzyl)amino)-8-ethyl-4-oxo-4,6,7,8-tetrahydropyrrolo[1,2-a]pyrazine-6-carboxylic acid (quantitative yield) as an oil.

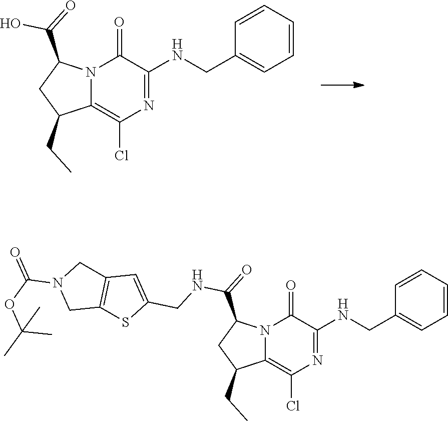 [SMILES: CC[C@H]1C[C@@H](C(=O)O)n2c1c(Cl)nc(NCc1ccccc1)c2=O ; CC[C@H]1C[C@@H](C(=O)NCc2cc3c(s2)CN(C(=O)OC(C)(C)C)C3)n2c1c(Cl)nc(NCc1ccccc1)c2=O]

Step 2: The above compound was synthesized according to General Amide Coupling Procedure B using the appropriate amine to afford tert-butyl 2-(((6S,8S)-3-(benzylamino)-8-ethyl-4-oxo-4,6,7,8-tetrahydropyrrolo[1,2-a]pyrazine-6-carboxamido)methyl)-4,6-dihydro-5H-thieno[2,3-c]pyrrole-5-carboxylate (74 mg, 71% yield) as an oil. P

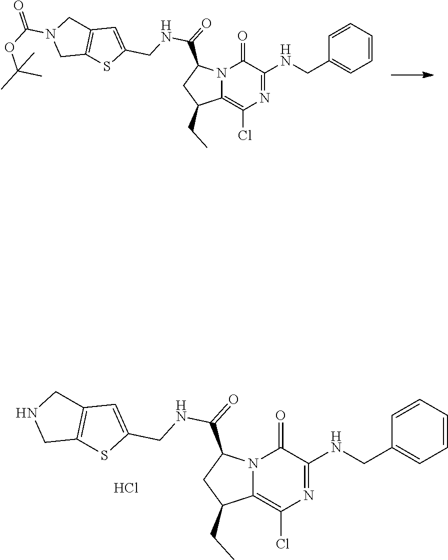 [SMILES: CC[C@H]1C[C@@H](C(=O)NCc2cc3c(s2)CN(C(=O)OC(C)(C)C)C3)n2c1c(Cl)nc(NCc1ccccc1)c2=O ; CC[C@H]1C[C@@H](C(=O)NCc2cc3c(s2)CNC3)n2c1c(Cl)nc(NCc1ccccc1)c2=O ; Cl]

Step 3: tert-Butyl 2-(((6S,8S)-3-(benzylamino)-1-chloro-8-ethyl-4-oxo-4,6,7,8-tetrahydropyrrolo[1,2-a]pyrazine-6-carboxamido)methyl)-4,6-dihydro-5H-thieno[2,3-c]pyrrole-5-carboxylate (21 mg, 0.036 mmol) was stirred in a solution of saturated HCl in MeOH (3 mL) for 4 h. The solution was evaporated to dryness followed by lyophilization from H₂O (2 mL) to afford (6S,8S)-3-(benzylamino)-1-chloro-N-((5,6-dihydro-4H-thieno[2,3-c]pyrrol-2-yl)methyl)-8-ethyl-4-oxo-4,6,7,8-tetrahydropyrrolo[1,2-a]pyrazine-6-carboxamide hydrochloride (17 mg, 91% yield) as a fluffy white solid.

### Example 29

### Preparation of (6S,8S)—N-((1H-pyrrolo[3,2-c]pyridin-2-yl)methyl)-3-(benzylamino)-1-chloro-8-ethyl-4-oxo-4,6,7,8-tetrahydropyrrolo[1,2-a]pyrazine-6-carboxamide

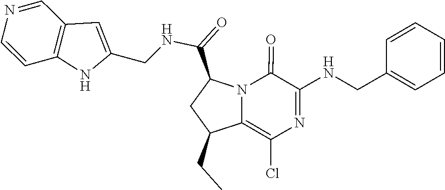 [SMILES: CC[C@H]1C[C@@H](C(=O)NCc2cc3cnccc3[nH]2)n2c1c(Cl)nc(NCc1ccccc1)c2=O]

Steps 1-2: The above compound was synthesized with the appropriate starting materials according to Example 28, steps 1-2.

The following compound was prepared according to the foregoing procedures using the appropriate amine:

| | Cmp |
|---|---|
| Compound Name | No. |
| (6S,8S)-N-((1H-pyrrolo[3,2-c]pyridin-2-yl)methyl)-1-chloro-3- | 160 |
| ((3,5-dimethylbenzyl)amino)-8-ethyl-4-oxo-4,6,7,8- | |
| tetrahydropyrrolo[1,2-a]pyrazine-6-carboxamide | |

### Example 30

### Preparation of (6S,8S)-3-(benzylamino)-N-((5,6-dihydro-4H-thieno[2,3-c]pyrrol-2-yl)methyl)-8-ethyl-4-oxo-4,6,7,8-tetrahydropyrrolo[1,2-a]pyrazine-6-carboxamide hydrochloride

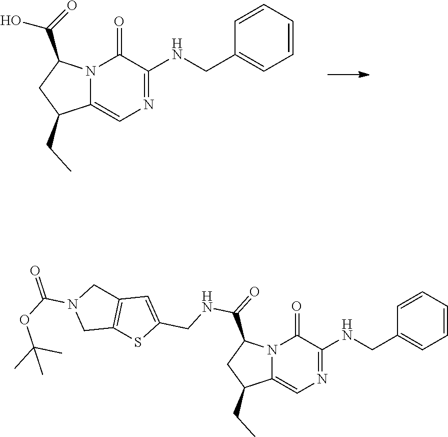 [SMILES: CC[C@H]1C[C@@H](C(=O)O)n2c1cnc(NCc1ccccc1)c2=O ; CC[C@H]1C[C@@H](C(=O)NCc2cc3c(s2)CN(C(=O)OC(C)(C)C)C3)n2c1cnc(NCc1ccccc1)c2=O]

Step 1: To a solution of (6S,8S)-3-(benzylamino)-8-ethyl-4-oxo-4,6,7,8-tetrahydropyrrolo[1,2-a]pyrazine-6-carboxylic acid (60 mg, 0.19 mmol) in DMF (2 mL) was added NHS (24 mg, 0.21 mmol), DCC (43 mg, 0.21 mmol), and tert-butyl 2-(aminomethyl)-4,6-dihydro-5H-thieno[2,3-c]pyrrole-5-carboxylate (54 mg, 0.21 mmol). The solution was stirred for 16 h at room temp and then evaporated to dryness. The residue was dissolved in 5% MeOH—CH₂Cl₂ (5 mL) and filtered to remove solids. Evaporation to dryness and purification by chromatography (5% MeOH (7M NH₃)—CH₂Cl₂) gave tert-butyl 2-(((6S,8S)-3-(benzylamino)-8-ethyl-4-oxo-4,6,7,8-tetrahydropyrrolo[1,2-a]pyrazine-6-carboxamido)methyl)-4,6-dihydro-5H-thieno[2,3-c]pyrrole-5-carboxylate (74 mg, 71% yield) as an oil.

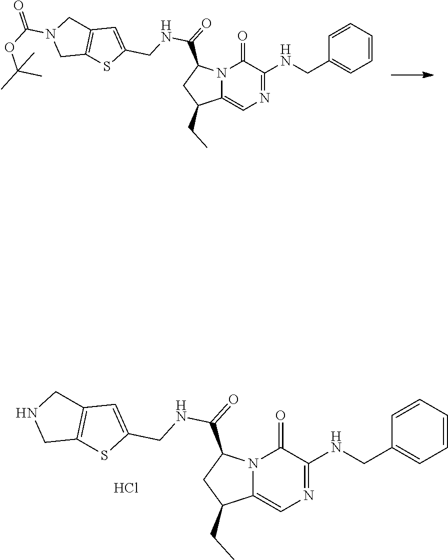 [SMILES: CC[C@H]1C[C@@H](C(=O)NCc2cc3c(s2)CN(C(=O)OC(C)(C)C)C3)n2c1cnc(NCc1ccccc1)c2=O ; CC[C@H]1C[C@@H](C(=O)NCc2cc3c(s2)CNC3)n2c1cnc(NCc1ccccc1)c2=O ; Cl]

Step 2: tert-Butyl 2-(((6S,8S)-3-(benzylamino)-8-ethyl-4-oxo-4,6,7,8-tetrahydropyrrolo[1,2-a]pyrazine-6-carboxamido)methyl)-4,6-dihydro-5H-thieno[2,3-c]pyrrole-5-carboxylate (74 mg, 0.13 mmol) was stirred in a solution of saturated HCl in MeOH (3 mL) for 4 h. Evaporation to dryness followed by lyophilization from H₂O gave (6S,8S)-3-(benzylamino)-N-((5,6-dihydro-4H-thieno[2,3-c]pyrrol-2-yl)methyl)-8-ethyl-4-oxo-4,6,7,8-tetrahydropyrrolo[1,2-a]pyrazine-6-carboxamide hydrochloride (63 mg, 96% yield) as a fluffy white solid.

### Example 31

### Preparation of (S)—N-((1H-pyrrolo[3,2-c]pyridin-2-yl)methyl)-1-chloro-4-oxo-3-(((5,6,7,8-tetrahydronaphthalen-2-yl)methyl)amino)-4,6,7,8-tetrahydropyrrolo[1,2-a]pyrazine-6-carboxamide

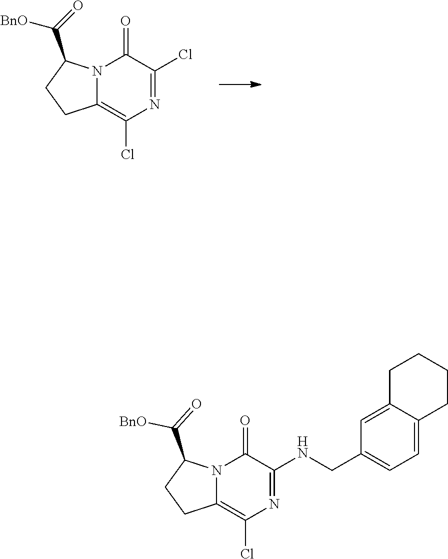 [SMILES: O=C(OCc1ccccc1)[C@@H]1CCc2c(Cl)nc(Cl)c(=O)n21 ; O=C(OCc1ccccc1)[C@@H]1CCc2c(Cl)nc(NCc3ccc4c(c3)CCCC4)c(=O)n21]

Step 1: The above compound was prepared according to General Chloro Displacement Procedure A to afford benzyl (S)-1-chloro-4-oxo-3-(((5,6,7,8-tetrahydronaphthalen-2-yl)methyl)amino)-4,6,7,8-tetrahydropyrrolo[1,2-a]pyrazine-6-carboxylate (166 mg, 81% yield).

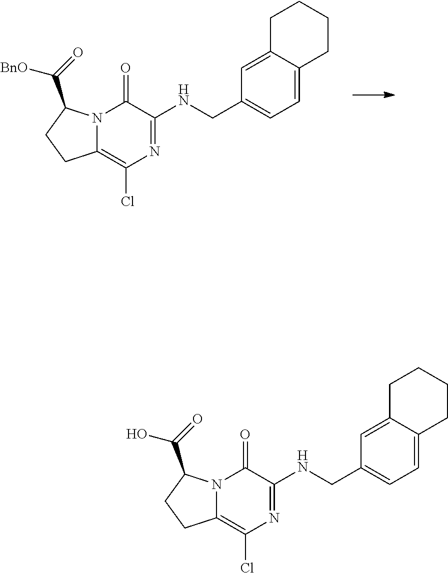 [SMILES: O=C(OCc1ccccc1)[C@@H]1CCc2c(Cl)nc(NCc3ccc4c(c3)CCCC4)c(=O)n21 ; O=C(O)[C@@H]1CCc2c(Cl)nc(NCc3ccc4c(c3)CCCC4)c(=O)n21]

Step 2: To a solution of benzyl (S)-1-chloro-4-oxo-3-(((5,6,7,8-tetrahydronaphthalen-2-yl)methyl)amino)-4,6,7,8-tetrahydropyrrolo[1,2-a]pyrazine-6-carboxylate (166 mg, 358 μmol) in THF (5 mL) and MeOH (2.5 mL), was added LiOH (42.8 mg, 1.74 mmol) in H₂O (2.5 mL) with stirring for 4 h at room temp. The solution was evaporated to dryness and H₂O (10 mL) was added with stirring. The pH was adjusted to approximately 2 with the dropwise addition of concentrated HCl. The product was collected by filtration, washed with H₂O (2×2 mL), and dried, giving benzyl (S)-3-(([1,1′-biphenyl]-3-ylmethyl)amino)-1-chloro-4-oxo-4,6,7,8-tetrahydropyrrolo[1,2-a]pyrazine-6-carboxylate (108 mg, 81% yield) as a pale yellow solid.

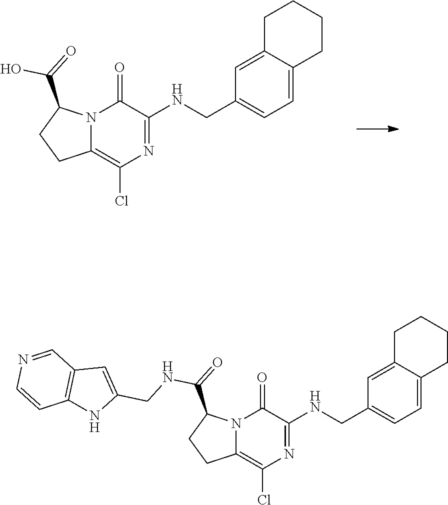 [SMILES: O=C(O)[C@@H]1CCc2c(Cl)nc(NCc3ccc4c(c3)CCCC4)c(=O)n21 ; O=C(NCc1cc2cnccc2[nH]1)[C@@H]1CCc2c(Cl)nc(NCc3ccc4c(c3)CCCC4)c(=O)n21]

Step 3: To a solution of (S)-1-chloro-4-oxo-3-(((5,6,7,8-tetrahydronaphthalen-2-yl)methyl)amino)-4,6,7,8-tetrahydropyrrolo[1,2-a]pyrazine-6-carboxylic acid (40 mg, 0.11 mmol) in DMF (2 mL) was added NHS (12 mg, 0.11 mmol), DCC (22 mg, 0.11 mmol), and (1H-pyrrolo[3,2-c]pyridin-2-yl)methanamine (16 mg, 0.11 mmol). The solution was stirred for 16 h at room temp then evaporated to dryness. The residue was dissolved in 5% MeOH—CH₂Cl₂ (5 mL) and filtered to remove solids. The solution was evaporated to dryness, then purified by chromatography (4% MeOH (7M NH₃)—CH₂Cl₂) to afford (S)—N-((1H-pyrrolo[3,2-c]pyridin-2-yl)methyl)-1-chloro-4-oxo-3-(((5,6,7,8-tetrahydronaphthalen-2-yl)methyl)amino)-4,6,7,8-tetrahydropyrrolo[1,2-a]pyrazine-6-carboxamide (42 mg, 78% yield) as a pale yellow solid.

### Example 32

### Preparation of (R)—N-((6-amino-2-methylpyridin-3-yl)methyl)-3-((3-fluoro-5-methylbenzyl)amino)-4-oxo-4,6,7,8-tetrahydropyrrolo[1,2-a]pyrazine-6-carboxamide

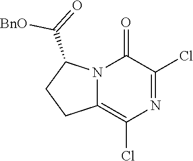 [SMILES: O=C(OCc1ccccc1)[C@H]1CCc2c(Cl)nc(Cl)c(=O)n21]

Steps 1-4: The above compound was synthesized with the appropriate starting materials according to Example 2, steps 1-4.

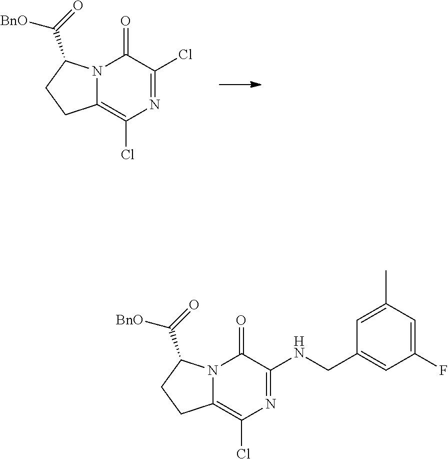 [SMILES: O=C(OCc1ccccc1)[C@H]1CCc2c(Cl)nc(Cl)c(=O)n21 ; Cc1cc(F)cc(CNc2nc(Cl)c3n(c2=O)[C@@H](C(=O)OCc2ccccc2)CC3)c1]

Step 5: To a solution of benzyl (R)-1,3-dichloro-4-oxo-4,6,7,8-tetrahydropyrrolo[1,2-a]pyrazine-6-carboxylate (150 mg, 0.44 mmol) in MeCN (5 mL) was added DIEA (114 mg, 0.89 mmol) and (3-fluoro-5-methylphenyl)methanamine (123 mg, 0.89 mmol). The solution was heated at 70° C. for 16 h then evaporated to dryness. Chromatography (30% EtOAc-hexanes) gave benzyl (R)-1-chloro-3-((3-fluoro-5-methylbenzyl)amino)-4-oxo-4,6,7,8-tetrahydropyrrolo[1,2-a]pyrazine-6-carboxylate (109 mg, 56% yield) as an oil.

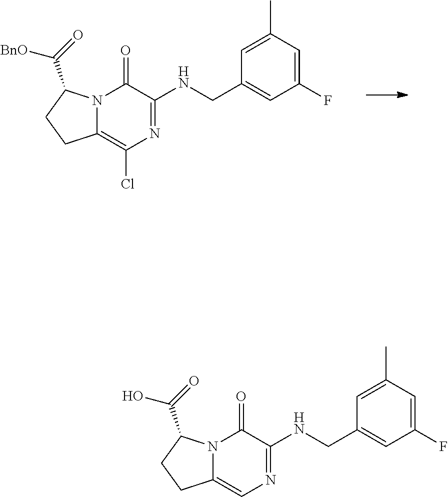 [SMILES: Cc1cc(F)cc(CNc2nc(Cl)c3n(c2=O)[C@@H](C(=O)OCc2ccccc2)CC3)c1 ; Cc1cc(F)cc(CNc2ncc3n(c2=O)[C@@H](C(=O)O)CC3)c1]

Step 6: To a solution of benzyl (R)-1-chloro-3-((3-methoxy-5-methylbenzyl)amino)-4-oxo-4,6,7,8-tetrahydropyrrolo[1,2-a]pyrazine-6-carboxylate (109 mg, 0.25 mmol) in degassed MeOH (5 mL) was added DIEA (64 mg, 0.49 mmol) and 10% Pd/C (30 mg) with stirring. This solution was placed under H₂ (120 psi) for 48 h. The catalyst was removed by filtration followed by evaporation, giving (R)-3-((3-fluoro-5-methylbenzyl)amino)-4-oxo-4,6,7,8-tetrahydropyrrolo[1,2-a]pyrazine-6-carboxylic acid (78 mg, quantitative yield).

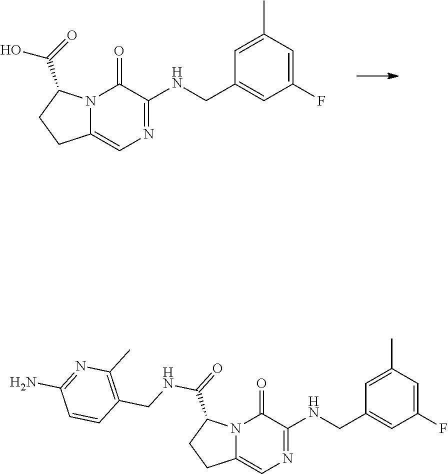 [SMILES: Cc1cc(F)cc(CNc2ncc3n(c2=O)[C@@H](C(=O)O)CC3)c1 ; Cc1cc(F)cc(CNc2ncc3n(c2=O)[C@@H](C(=O)NCc2ccc(N)nc2C)CC3)c1]

Step 7: To a solution of (R)-3-((3-fluoro-5-methylbenzyl)amino)-4-oxo-4,6,7,8-tetrahydropyrrolo[1,2-a]pyrazine-6-carboxylic acid (78 mg, 0.25 mmol) in DMF (2 mL) was added DIEA (35 mg, 0.27 mmol), HATU (100 mg, 0.27 mmol) and 5-(aminomethyl)-6-methylpyridin-2-amine (40 mg, 0.29 mmol). The solution was stirred for 16 h at room temp, then evaporated to dryness and purified by chromatography (5% MeOH (7M NH₃)—CH₂Cl₂) to give (R)—N-((6-amino-2-methylpyridin-3-yl)methyl)-3-((3-fluoro-5-methylbenzyl)amino)-4-oxo-4,6,7,8-tetrahydropyrrolo[1,2-a]pyrazine-6-carboxamide (30 mg, 28% yield) as a pale yellow solid.

The following compound was prepared according to the foregoing procedures using the appropriate amine:

| | Cmp |
|---|---|
| Compound Name | No. |
| (R)-N-((1H-pyrrolo[3,2-c]pyridin-2-yl)methyl)-3-((3-methoxy-5- | 164 |
| methylbenzyl)amino)-4-oxo-4,6,7,8-tetrahydropyrrolo[1,2- | |
| a]pyrazine-6-carboxamide | |

### Example 33

### Preparation of(S)-1-chloro-3-((3-fluoro-5-methylbenzyl)amino)-N-(imidazo[1,2-a]pyridin-3-ylmethyl)-4-oxo-4,6,7,8-tetrahydropyrrolo[1,2-a]pyrazine-6-carboxamide trifluoroacetate

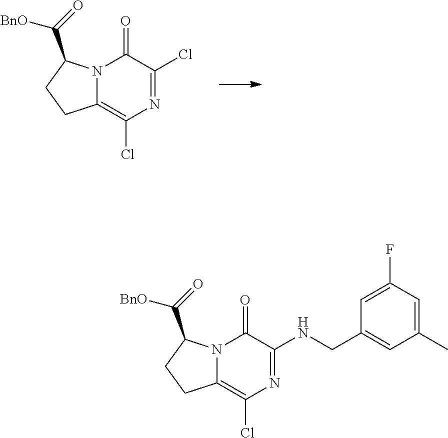 [SMILES: O=C(OCc1ccccc1)[C@@H]1CCc2c(Cl)nc(Cl)c(=O)n21 ; Cc1cc(F)cc(CNc2nc(Cl)c3n(c2=O)[C@H](C(=O)OCc2ccccc2)CC3)c1]

Step 1: The above compound was synthesized according to General Chloro Displacement Procedure A to afford benzyl (S)-1-chloro-3-((3-fluoro-5-methylbenzyl)amino)-4-oxo-4,6,7,8-tetrahydropyrrolo[1,2-a]pyrazine-6-carboxylate (1.01 g, 73% yield) as a yellow solid.

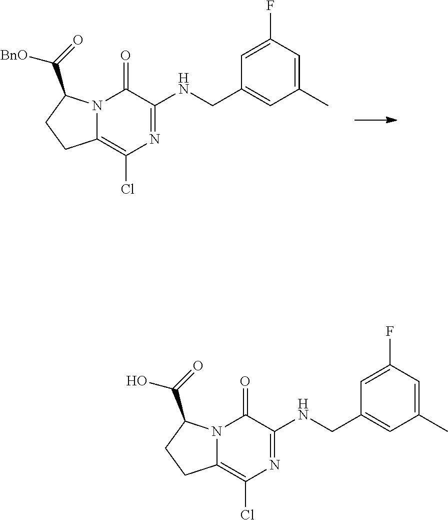 [SMILES: Cc1cc(F)cc(CNc2nc(Cl)c3n(c2=O)[C@H](C(=O)OCc2ccccc2)CC3)c1 ; Cc1cc(F)cc(CNc2nc(Cl)c3n(c2=O)[C@H](C(=O)O)CC3)c1]

Step 2: The above compound was synthesized according to General Hydrogenolysis Procedure A, except that crude material was loaded onto a silica gel column and eluted with EtOAc-heptane to afford (S)-1-chloro-3-((3-fluoro-5-methylbenzyl)amino)-4-oxo-4,6,7,8-tetrahydropyrrolo[1,2-a]pyrazine-6-carboxylic acid (78.1 mg, 27% yield) as a clear oil.

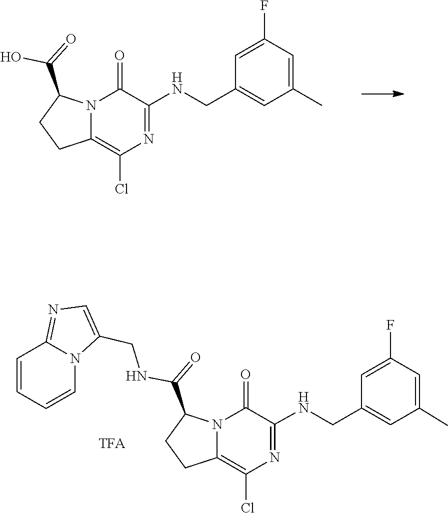 [SMILES: Cc1cc(F)cc(CNc2nc(Cl)c3n(c2=O)[C@H](C(=O)O)CC3)c1 ; Cc1cc(F)cc(CNc2nc(Cl)c3n(c2=O)[C@H](C(=O)NCc2cnc4ccccn24)CC3)c1]

Step 3: The above compound was synthesized according to General Amide Coupling Procedure A, except that the crude material was purified by prep HPLC to afford (S)-1-chloro-3-((3-fluoro-5-methylbenzyl)amino)-N-(imidazo[1,2-a]pyridin-3-ylmethyl)-4-oxo-4,6,7,8-tetrahydropyrrolo[1,2-a]pyrazine-6-carboxamide trifluoroacetate as a white solid (44 mg, 77% yield).

### Example 34

### Preparation of (S)—N-((6-amino-2-methylpyridin-3-yl)methyl)-4-oxo-3-(((1-phenyl-1h-pyrazol-4-yl)methyl)amino)-4,6,7,8-tetrahydropyrrolo[1,2-a]pyrazine-6-carboxamide trifluoroacetate

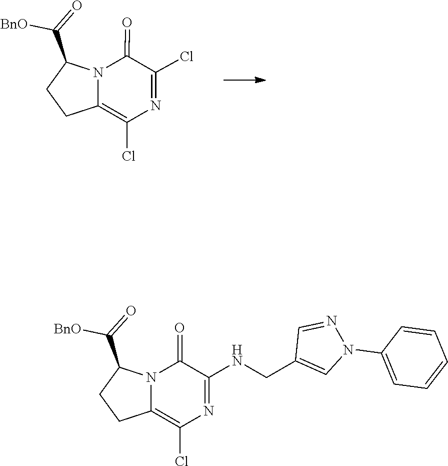 [SMILES: O=C(OCc1ccccc1)[C@@H]1CCc2c(Cl)nc(Cl)c(=O)n21 ; O=C(OCc1ccccc1)[C@@H]1CCc2c(Cl)nc(NCc3cnn(-c4ccccc4)c3)c(=O)n21]

Step 1: The above compound was synthesized according to General Chloro Displacement Procedure A to afford benzyl (S)-1-chloro-4-oxo-3-(((1-phenyl-1H-pyrazol-4-yl)methyl)amino)-4,6,7,8-tetrahydropyrrolo[1,2-a]pyrazine-6-carboxylate as a light-yellow solid 129 mg, 92% yield).

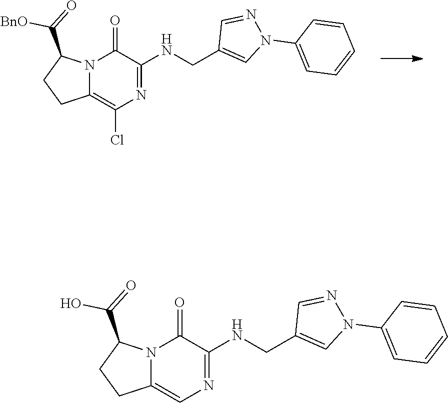 [SMILES: O=C(OCc1ccccc1)[C@@H]1CCc2c(Cl)nc(NCc3cnn(-c4ccccc4)c3)c(=O)n21 ; O=C(O)[C@@H]1CCc2cnc(NCc3cnn(-c4ccccc4)c3)c(=O)n21]

Step 2: The above compound was synthesized according to General Hydrogenolysis Procedure B, except that the reaction mixture was sonicated in anhydrous MeOH, at a concentration of 0.04 M, for about 5 min before adding DIEA and Pd/C. Crude (S)-4-oxo-3-(((1-phenyl-1H-pyrazol-4-yl)methyl)amino)-4,6,7,8-tetrahydropyrrolo[1,2-a]pyrazine-6-carboxylic acid was isolated as a thick clear oil (quantitative yield).

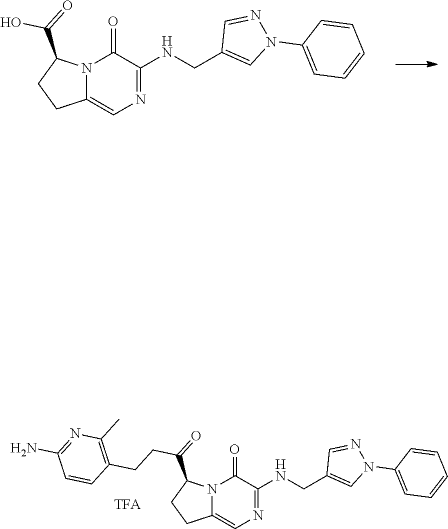 [SMILES: O=C(O)[C@@H]1CCc2cnc(NCc3cnn(-c4ccccc4)c3)c(=O)n21 ; Cc1nc(N)ccc1CCC(=O)[C@@H]1CCc2cnc(NCc3cnn(-c4ccccc4)c3)c(=O)n21]

Step 3: The above compound was synthesized according to General Amide Coupling Procedure A, except that the crude material was purified by prep HPLC to afford (S)—N-((6-amino-2-methylpyridin-3-yl)methyl)-4-oxo-3-(((1-phenyl-1H-pyrazol-4-yl)methyl)amino)-4,6,7,8-tetrahydropyrrolo[1,2-a]pyrazine-6-carboxamide trifluoroacetate as a white solid (50 mg, 38% yield).

The following compounds were prepared according to the foregoing procedures using the appropriate amines:

| | Cmp |
|---|---|
| Compound Name | No. |
| (S)-N-((6-amino-2-methylpyridin-3-yl)methyl)-3-(((1-methyl-1H- | 167 |
| pyrazol-4-yl)methyl)amino)-4-oxo-4,6,7,8-tetrahydropyrrolo[1,2- | |
| a]pyrazine-6-carboxamide trifluoroacetate | |
| (S)-N-((6-amino-2-methylpyridin-3-yl)methyl)-4-oxo-3-(((5,6,7,8- | 168 |
| tetrahydroimidazo[1,2-a]pyridin-2-yl)methyl)amino)-4,6,7,8- | |
| tetrahydropyrrolo[1,2-a]pyrazine-6-carboxamide trifluoroacetate | |
| (S)-3-((3-(1H-pyrazol-1-yl)benzyl)amino)-N-((6-amino-2- | 169 |
| methylpyridin-3-yl)methyl)-4-oxo-4,6,7,8-tetrahydropyrrolo[1,2- | |
| a]pyrazine-6-carboxamide trifluoroacetate | |
| (S)-N-((6-amino-2-methylpyridin-3-yl)methyl)-3-(((1-methyl-1H- | 170 |
| indazol-3-yl)methyl)amino)-4-oxo-4,6,7,8-tetrahydropyrrolo[1,2- | |
| a]pyrazine-6-carboxamide trifluoroacetate | |
| (S)-N-((6-amino-2-methylpyridin-3-yl)methyl)-3-((3-fluoro-5- | 171 |
| methoxybenzyl)amino)-4-oxo-4,6,7,8-tetrahydropyrrolo[1,2- | |
| a]pyrazine-6-carboxamide trifluoroacetate | |

The following compound was prepared according to the foregoing procedure using the appropriate amine, except that amine column eluting with a DCM-MeOH gradient was performed after HPLC-prep:

| | Cmp |
|---|---|
| Compound Name | No. |
| (S)-N-((6-amino-2-methylpyridin-3-yl)methyl)-3-(((1-methyl-1H- | 172 |
| indazol-5-yl)methyl)amino)-4-oxo-4,6,7,8-tetrahydropyrrolo[1,2- | |
| a]pyrazine-6-carboxamide | |

### Example 35

### Preparation of (S)—N-(4-((R)-1-aminoethyl)benzyl)-3-((3-fluoro-5-methylbenzyl)amino)-4-oxo-4,6,7,8-tetrahydropyrrolo[1,2-a]pyrazine-6-carboxamide trifluoroacetate

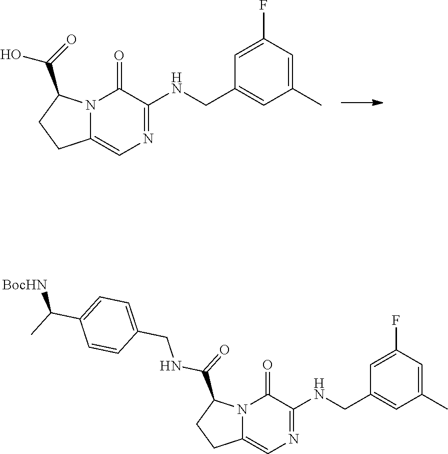

Step 1: The above compound was synthesized according to General Amide Coupling Procedure A to afford tert-butyl ((R)-1-(4-(((S)-3-((3-fluoro-5-methylbenzyl)amino)-4-oxo-4,6,7,8-tetrahydropyrrolo[1,2-a]pyrazine-6-carboxamido)methyl)phenyl)ethyl)carbamate (66 mg, 82% yield).

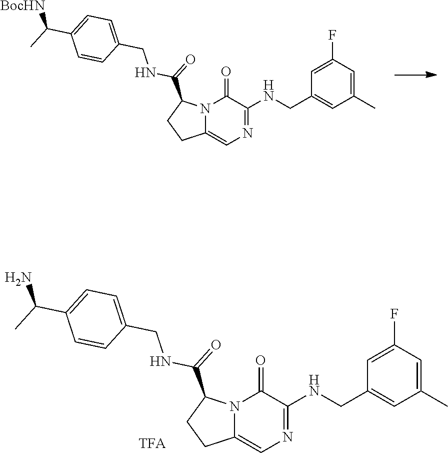

Step 2: The above compound was synthesized according to General Boc Removal Procedure A, except TES (4 eq) was used with 50% TFA and DCM (v/v). The crude material was purified by prep HPLC to afford (S)—N-(4-((R)-1-aminoethyl)benzyl)-3-((3-fluoro-5-methylbenzyl)amino)-4-oxo-4,6,7,8-tetrahydropyrrolo[1,2-a]pyrazine-6-carboxamide trifluoroacetate as a white solid (39 mg, 58% yield).

### Example 36

### Preparation of (S)—N-((6-amino-2-methylpyridin-3-yl)methyl)-3-((3-fluoro-5-methylbenzyl)amino)-1-(1-methyl-1H-pyrazol-4-yl)-4-oxo-4,6,7,8-tetrahydropyrrolo[1,2-a]pyrazine-6-carboxamide trifluoroacetate

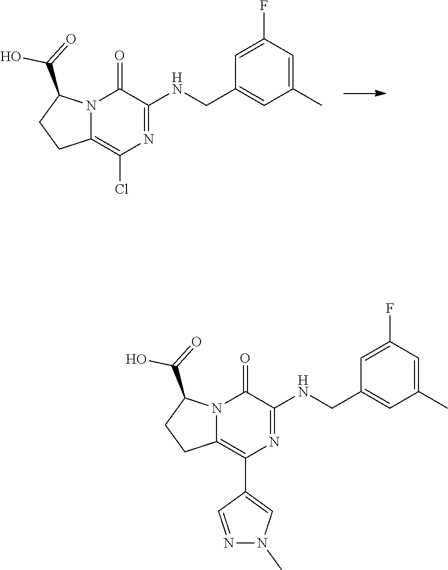 [SMILES: Cc1cc(F)cc(CNc2nc(Cl)c3n(c2=O)[C@H](C(=O)O)CC3)c1 ; Cc1cc(F)cc(CNc2nc(-c3cnn(C)c3)c3n(c2=O)[C@H](C(=O)O)CC3)c1]

Step 1: The above compound was synthesized according to General Suzuki-Miyaura Reaction Procedure A to afford (S)-3-((3-fluoro-5-methylbenzyl)amino)-1-(1-methyl-1H-pyrazol-4-yl)-4-oxo-4,6,7,8-tetrahydropyrrolo[1,2-a]pyrazine-6-carboxylic acid (60 mg, 64% yield).

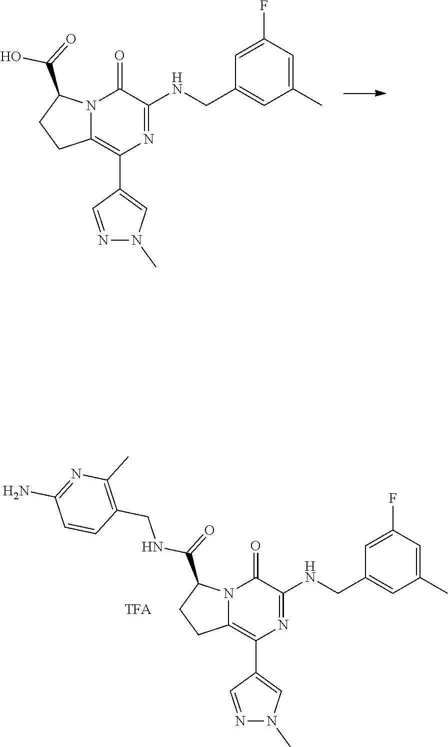 [SMILES: Cc1cc(F)cc(CNc2nc(-c3cnn(C)c3)c3n(c2=O)[C@H](C(=O)O)CC3)c1 ; Cc1cc(F)cc(CNc2nc(-c3cnn(C)c3)c3n(c2=O)[C@H](C(=O)NCc2ccc(N)nc2C)CC3)c1]

Step 2: The above compound was synthesized according to General Amide Coupling Procedure A, except that the crude material was purified by prep HPLC to afford (S)—N-((6-amino-2-methylpyridin-3-yl)methyl)-3-((3-fluoro-5-methylbenzyl)amino)-1-(1-methyl-1H-pyrazol-4-yl)-4-oxo-4,6,7,8-tetrahydropyrrolo[1,2-a]pyrazine-6-carboxamide trifluoroacetate as a white solid (53 mg, 56% yield).

The following compound was prepared according to the foregoing procedures using the appropriate-amine:

| | Cmp |
|---|---|
| Compound Name | No. |
| (S)-N-((6-amino-2-methylpyridin-3-yl)methyl)-3-((3-fluoro-5- | 175 |
| methylbenzyl)amino)-1-(1-methyl-1H-pyrazol-5-yl)-4-oxo-4,6,7,8- | |
| tetrahydropyrrolo[1,2-a]pyrazine-6-carboxamide trifluoroacetate | |

### Example 37

### Preparation of (S)—N-((6-amino-2-methylpyridin-3-yl)methyl)-1-cyclohexyl-3-((3-fluoro-5-methylbenzyl)amino)-4-oxo-4,6,7,8-tetrahydropyrrolo[1,2-a]pyrazine-6-carboxamide DI-trifluoroacetate

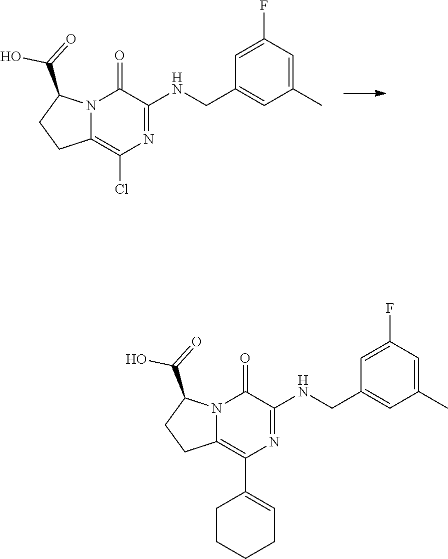 [SMILES: Cc1cc(F)cc(CNc2nc(Cl)c3n(c2=O)[C@H](C(=O)O)CC3)c1 ; Cc1cc(F)cc(CNc2nc(C3=CCCCC3)c3n(c2=O)[C@H](C(=O)O)CC3)c1]

Step 1: The above compound was synthesized according to General Suzuki-Miyaura Reaction Procedure B to afford (S)-1-(cyclohex-1-en-1-yl)-3-((3-fluoro-5-methylbenzyl)amino)-4-oxo-4,6,7,8-tetrahydropyrrolo[1,2-a]pyrazine-6-carboxylic acid (110 mg, 100% yield).

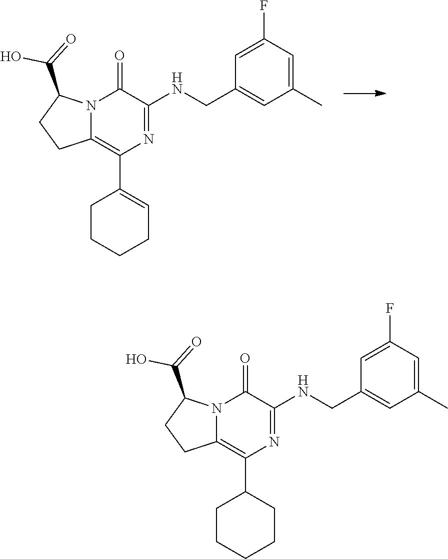 [SMILES: Cc1cc(F)cc(CNc2nc(C3=CCCCC3)c3n(c2=O)[C@H](C(=O)O)CC3)c1 ; Cc1cc(F)cc(CNc2nc(C3CCCCC3)c3n(c2=O)[C@H](C(=O)O)CC3)c1]

Step 2: The above compound was synthesized according to General Hydrogenolysis Procedure A to afford (S)-1-cyclohexyl-3-((3-fluoro-5-methylbenzyl)amino)-4-oxo-4,6,7,8-tetrahydropyrrolo[1,2-a]pyrazine-6-carboxylic acid (quantitative yield).

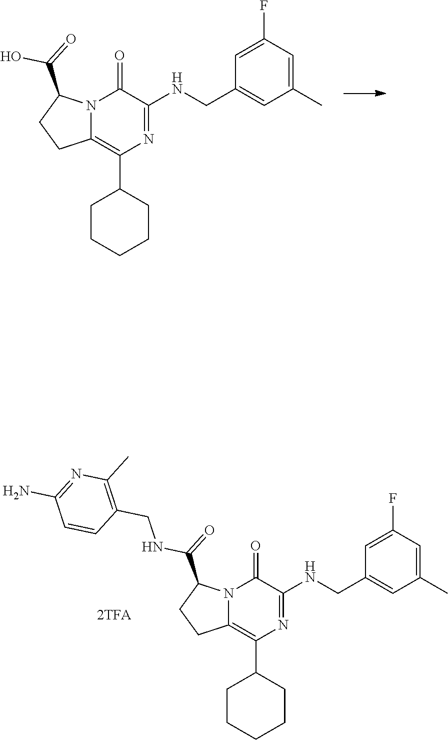 [SMILES: Cc1cc(F)cc(CNc2nc(C3CCCCC3)c3n(c2=O)[C@H](C(=O)O)CC3)c1 ; Cc1cc(F)cc(CNc2nc(C3CCCCC3)c3n(c2=O)[C@H](C(=O)NCc2ccc(N)nc2C)CC3)c1]

Step 3: The above compound was synthesized according to General Amide Coupling Procedure A, except that the crude material was purified by prep HPLC to afford (S)—N-((6-amino-2-methylpyridin-3-yl)methyl)-1-cyclohexyl-3-((3-fluoro-5-methylbenzyl)amino)-4-oxo-4,6,7,8-tetrahydropyrrolo[1,2-a]pyrazine-6-carboxamide di-trifluoroacetate (34 mg, 34% yield).

The following compounds were prepared according to the foregoing procedures sing the appropriate amines

| | Cmp |
|---|---|
| Compound Name | No. |
| (S)-N-((1H-pyrrolo[3,2-c]pyridin-2-yl)methyl)-1-cyclohexyl-3-((3- | 177 |
| fluoro-5-methylbenzyl)amino)-4-oxo-4,6,7,8-tetrahydropyrrolo[1,2- | |
| a]pyrazine-6-carboxamide trifluoroacetate | |

### Example 38

### Preparation of (S)—N—((R)-1-(4-(aminomethyl)phenyl)ethyl)-3-((3-fluoro-5-methylbenzyl)amino)-4-oxo-4,6,7,8-tetrahydropyrrolo[1,2-a]pyrazine-6-carboxamide trifluoroacetate

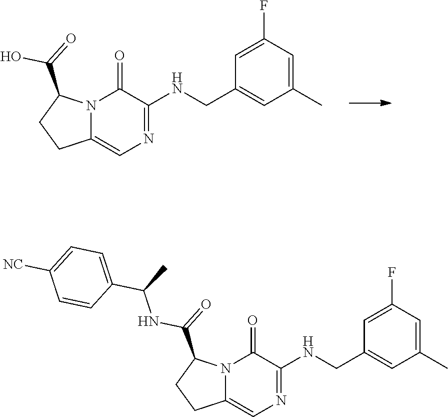 [SMILES: Cc1cc(F)cc(CNc2ncc3n(c2=O)[C@H](C(=O)O)CC3)c1 ; Cc1cc(F)cc(CNc2ncc3n(c2=O)[C@H](C(=O)N[C@H](C)c2ccc(C#N)cc2)CC3)c1]

Step 1: NHS (19 mg, 0.17 mmol) and DCC (34 mg, 0.172 mmol) were added to a solution of acid (39.6 mg, 0.125 mmol) in 1 mL DMF. The reaction mixture was stirred at room temp for 1.5 h. A solution of amine (33.3 mg, 0.228 mmol) in 0.9 mL DMF was added. The reaction was stirred at room temp for 16 h. Volatiles were evaporated under reduced pressure. The crude material was dissolved in CH₂Cl₂ and adsorbed onto silica gel. Purification by chromatography using MeOH-DCM gradient was undertaken to afford (S)—N—((R)-1-(4-cyanophenyl)ethyl)-3-((3-fluoro-5-methylbenzyl)amino)-4-oxo-4,6,7,8-tetrahydropyrrolo[1,2-a]pyrazine-6-carboxamide (53 mg, yield 96% yield).

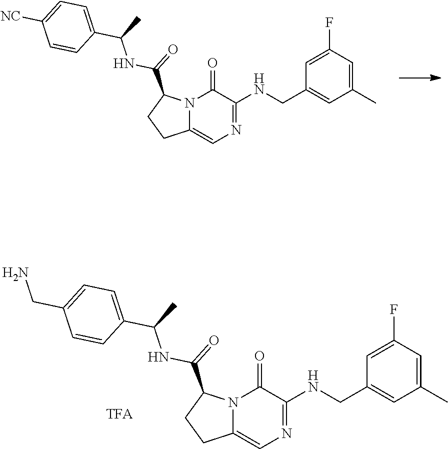 [SMILES: Cc1cc(F)cc(CNc2ncc3n(c2=O)[C@H](C(=O)N[C@H](C)c2ccc(C#N)cc2)CC3)c1 ; Cc1cc(F)cc(CNc2ncc3n(c2=O)[C@H](C(=O)N[C@H](C)c2ccc(CN)cc2)CC3)c1]

Step 2: The above compound was synthesized according to General Hydrogenolysis Procedure A, except that the aryl nitrile (1 eq) was dissolved in a solution of 7 N NH₃ in MeOH at a concentration of 0.1 M, and Raney®-Ni (7 eq) was used instead of Pd/C. The crude material was purified by prep HPLC to afford (S)—N—((R)-1-(4-(aminomethyl)phenyl)ethyl)-3-((3-fluoro-5-methylbenzyl)amino)-4-oxo-4,6,7,8-tetrahydropyrrolo[1,2-a]pyrazine-6-carboxamide trifluoroacetate (6 mg, 8% yield).

### Example 39

### Preparation of (S)—N-(5-chloro-2-(1H-tetrazol-1-yl)benzyl)-3-((3-fluoro-5-methylbenzyl)amino)-4-oxo-4,6,7,8-tetrahydropyrrolo[1,2-a]pyrazine-6-carboxamide

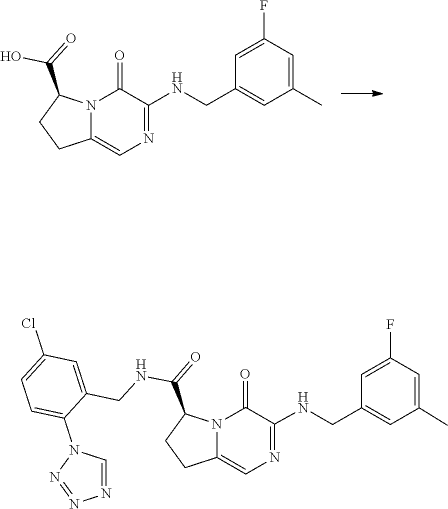 [SMILES: Cc1cc(F)cc(CNc2ncc3n(c2=O)[C@H](C(=O)O)CC3)c1 ; Cc1cc(F)cc(CNc2ncc3n(c2=O)[C@H](C(=O)NCc2cc(Cl)ccc2-n2cnnn2)CC3)c1]

Step 1: The above compound was synthesized according to General Amide Coupling Procedure B to afford (S)—N-(5-chloro-2-(1H-tetrazol-1-yl)benzyl)-3-((3-fluoro-5-methylbenzyl)amino)-4-oxo-4,6,7,8-tetrahydropyrrolo[1,2-a]pyrazine-6-carboxamide as a white solid (20 mg, 50% yield).

The following compound was prepared according to the foregoing procedures using the appropriate amine:

| | Cmp |
|---|---|
| Compound Name | No. |
| (S)-N-(3-chlorobenzyl)-3-((3-fluoro-5-methylbenzyl)amino)-4-oxo- | 180 |
| 4,6,7,8-tetrahydropyrrolo[1,2-a]pyrazine-6-carboxamide | |

### Example 40

### Preparation of (S)—N-(4-(aminomethyl)benzyl)-3-((3-fluoro-5-methylbenzyl)amino)-4-oxo-4,6,7,8-tetrahydropyrrolo[1,2-a]pyrazine-6-carboxamide trifluoroacetate

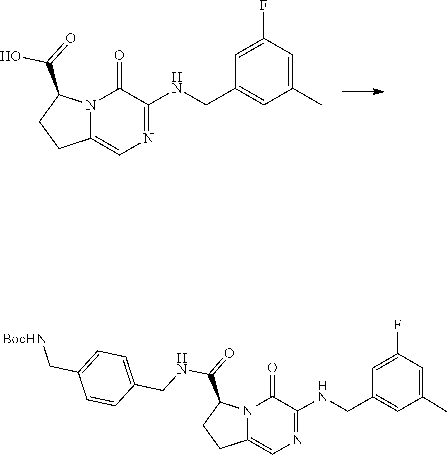 [SMILES: Cc1cc(F)cc(CNc2ncc3n(c2=O)[C@H](C(=O)O)CC3)c1 ; Cc1cc(F)cc(CNc2ncc3n(c2=O)[C@H](C(=O)NCc2ccc(CNC(=O)OC(C)(C)C)cc2)CC3)c1]

Step 1: The above compound was synthesized according to General Amide Coupling Procedure B to afford tert-butyl (S)-(4-((3-((3-fluoro-5-methylbenzyl)amino)-4-oxo-4,6,7,8-tetrahydropyrrolo[1,2-a]pyrazine-6-carboxamido)methyl)benzyl)carbamate as a white solid (40 mg, 79% yield).

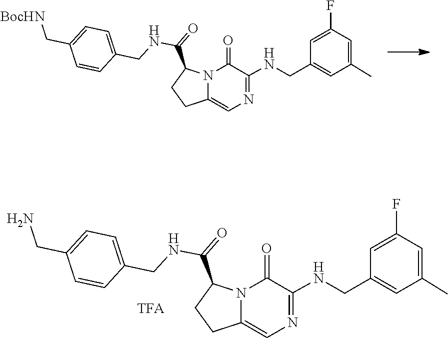 [SMILES: Cc1cc(F)cc(CNc2ncc3n(c2=O)[C@H](C(=O)NCc2ccc(CNC(=O)OC(C)(C)C)cc2)CC3)c1 ; Cc1cc(F)cc(CNc2ncc3n(c2=O)[C@H](C(=O)NCc2ccc(CN)cc2)CC3)c1]

Step 2: The above compound was synthesized according to General Boc Removal Procedure A, except that prep HPLC purification was performed to afford (S)—N-(4-(aminomethyl)benzyl)-3-((3-fluoro-5-methylbenzyl)amino)-4-oxo-4,6,7,8-tetrahydropyrrolo[1,2-a]pyrazine-6-carboxamide trifluoroacetate (34 mg, 83% yield).

### Example 41

### Preparation of (S)—N-((3-chloro-1H-pyrrolo[2,3-b]pyridin-5-yl)methyl)-3-((3-fluoro-5-methylbenzyl)amino)-4-oxo-4,6,7,8-tetrahydropyrrolo[1,2-a]pyrazine-6-carboxamide

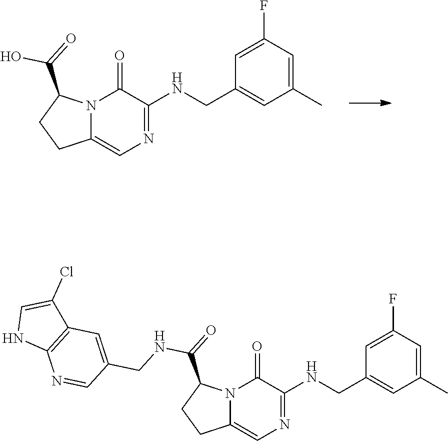 [SMILES: Cc1cc(F)cc(CNc2ncc3n(c2=O)[C@H](C(=O)O)CC3)c1 ; Cc1cc(F)cc(CNc2ncc3n(c2=O)[C@H](C(=O)NCc2cnc4[nH]cc(Cl)c4c2)CC3)c1]

Step 1: The above compound was synthesized according to General Amide Coupling Procedure A, except the final compound was purified using prep HPLC followed by an amine column eluting with a DCM-MeOH gradient to afford (S)—N-((3-chloro-1H-pyrrolo[2,3-b]pyridin-5-yl)methyl)-3-((3-fluoro-5-methylbenzyl)amino)-4-oxo-4,6,7,8-tetrahydropyrrolo[1,2-a]pyrazine-6-carboxamide as a white solid (4.5 mg, 30% yield).

The following compounds were prepared according to the foregoing procedures using the appropriate amines, except that HPLC purification was not used in the final step:

| | Cmp |
|---|---|
| Compound Name | No. |
| (S)-N-((1H-pyrrolo[2,3-b]pyridin-5-yl)methyl)-3-((3-fluoro-5- | 183 |
| methylbenzyl)amino)-4-oxo-4,6,7,8-tetrahydropyrrolo[1,2- | |
| a]pyrazine-6-carboxamide | |
| (S)-N-(4-amino-3-fluorobenzyl)-3-((3-fluoro-5- | 184 |
| methylbenzyl)amino)-4-oxo-4,6,7,8-tetrahydropyrrolo[1,2- | |
| a]pyrazine-6-carboxamide | |
| (6S)-N-((3-chlorophenyl)(cyano)methyl)-3-((3-fluoro-5- | 185 |
| methylbenzyl)amino)-4-oxo-4,6,7,8-tetrahydropyrrolo[1,2- | |
| a]pyrazine-6-carboxamide | |
| (S)-N-((R)-2-amino-6,7-dihydro-5H-cyclopenta[b]pyridin-5-yl)-3- | 186 |
| ((3,5-dimethylbenzyl)amino)-4-oxo-4,6,7,8-tetrahydropyrrolo[1,2- | |
| a]pyrazine-6-carboxamide | |
| (S)-N-((R)-1-(6-aminopyridin-3-yl)ethyl)-3-((3- | 187 |
| methylbenzyl)amino)-4-oxo-4,6,7,8-tetrahydropyrrolo[1,2- | |
| a]pyrazine-6-carboxamide | |
| (S)-N-(4-amino-3-chlorobenzyl)-3-((3-fluoro-5- | 188 |
| methylbenzyl)amino)-4-oxo-4,6,7,8-tetrahydropyrrolo[1,2- | |
| a]pyrazine-6-carboxamide | |
| (S)-N-((6-amino-5-chloropyridin-3-yl)methyl)-3-((3,5- | 189 |
| dimethylbenzyl)amino)-4-oxo-4,6,7,8-tetrahydropyrrolo[1,2- | |
| a]pyrazine-6-carboxamide | |

The following compounds were prepared according to the foregoing procedures using the appropriate amines, except that final purification consisted of only prep HPLC:

| | Cmp |
|---|---|
| Compound Name | No. |
| (S)-N-((3-fluoro-4-methoxypyridin-2-yl)methyl)-3-((3-fluoro-5- | 190 |
| methylbenzyl)amino)-4-oxo-4,6,7,8-tetrahydropyrrolo[1,2-a]pyrazine-6- | |
| carboxamide trifluoroacetate | |
| (S)-N-((R)-1-(6-aminopyridin-3-yl)ethyl)-3-((3-fluoro-5- | 191 |
| methylbenzyl)amino)-4-oxo-4,6,7,8-tetrahydropyrrolo[1,2-a]pyrazine-6- | |
| carboxamide trifluoroacetate | |
| (S)-N-((6-amino-5-chloropyridin-3-yl)methyl)-3-((3-fluoro-5- | 192 |
| methylbenzyl)amino)-4-oxo-4,6,7,8-tetrahydropyrrolo[1,2-a]pyrazine-6- | |
| carboxamide trifluoroacetate | |
| (S)-N-((R)-1-(6-aminopyridin-3-yl)ethyl)-3-((2,5-dimethylbenzyl)amino)-4- | 193 |
| oxo-4,6,7,8-tetrahydropyrrolo[1,2-a]pyrazine-6-carboxamide | |
| trifluoroacetate | |
| (S)-N-((R)-1-(6-aminopyridin-3-yl)ethyl)-3-((3-methoxy-5- | 194 |
| methylbenzyl)amino)-4-oxo-4,6,7,8-tetrahydropyrrolo[1,2-a]pyrazine-6- | |
| carboxamide trifluoroacetate | |
| (S)-N-((R)-1-(6-aminopyridin-3-yl)ethyl)-3-((3-ethylbenzyl)amino)-4-oxo- | 195 |
| 4,6,7,8-tetrahydropyrrolo[1,2-a]pyrazine-6-carboxamide trifluoroacetate | |
| (S)-N-((R)-1-(6-aminopyridin-3-yl)ethyl)-3-((5-fluoro-2- | 196 |
| methylbenzyl)amino)-4-oxo-4,6,7,8-tetrahydropyrrolo[1,2-a]pyrazine-6- | |
| carboxamide trifluoroacetate | |
| (S)-N-((R)-1-(6-aminopyridin-3-yl)ethyl)-3-((2-methyl-5- | 197 |
| (trifluoromethyl)benzyl)amino)-4-oxo-4,6,7,8-tetrahydropyrrolo[1,2- | |
| a]pyrazine-6-carboxamide trifluoroacetate | |
| (S)-N-((R)-1-(6-aminopyridin-3-yl)ethyl)-3-((3,5-dimethylbenzyl)amino)-4- | 198 |
| oxo-4,6,7,8-tetrahydropyrrolo[1,2-a]pyrazine-6-carboxamide | |
| trifluoroacetate | |
| (S)-N-((R)-1-(6-aminopyridin-3-yl)ethyl)-3-((2-fluoro-5- | 199 |
| methylbenzyl)amino)-4-oxo-4,6,7,8-tetrahydropyrrolo[1,2-a]pyrazine-6- | |
| carboxamide trifluoroacetate | |
| (S)-N-((R)-1-(6-aminopyridin-3-yl)ethyl)-3-((3-fluoro-5- | 200 |
| methoxybenzyl)amino)-4-oxo-4,6,7,8-tetrahydropyrrolo[1,2-a]pyrazine-6- | |
| carboxamide trifluoroacetate | |

The following compounds were prepared according to the foregoing procedures using the appropriate amines, except that prep HPLC purification followed by an amine column eluting with DCM-MeOH were used in the final step:

| | Cmp |
|---|---|
| Compound Name | No. |
| (S)-3-(([1,1′-biphenyl]-3-ylmethyl)amino)-N-((R)-1-(6- | 201 |
| aminopyridin-3-yl)ethyl)-4-oxo-4,6,7,8-tetrahydropyrrolo[1,2- | |
| a]pyrazine-6-carboxamide | |
| (S)-N-((R)-1-(6-aminopyridin-3-yl)ethyl)-3-((2- | 202 |
| methylbenzyl)amino)-4-oxo-4,6,7,8-tetrahydropyrrolo[1,2- | |
| a]pyrazine-6-carboxamide | |
| (S)-N-((R)-1-(6-aminopyridin-3-yl)ethyl)-3-((3-methyl-5- | 203 |
| (trifluoromethyl)benzyl)amino)-4-oxo-4,6,7,8- | |
| tetrahydropyrrolo[1,2-a]pyrazine-6-carboxamide | |

The following compound was prepared according to the foregoing procedures using the appropriate amine, except that prep HPLC purification was used in the final step:

| | Cmp |
|---|---|
| Compound Name | No. |
| (S)-3-((3-fluoro-5-methylbenzyl)amino)-N-(imidazo[1,2-a]pyridin- | 204 |
| 3-ylmethyl)-4-oxo-4,6,7,8-tetrahydropyrrolo[1,2-a]pyrazine-6- | |
| carboxamide trifluoroacetate | |

### Example 42

### Preparation of (S)—N-(4-(aminomethyl)-2-methylbenzyl)-3-((3-fluoro-5-methylbenzyl)amino)-4-oxo-4,6,7,8-tetrahydropyrrolo[1,2-a]pyrazine-6-carboxamide trifluoroacetate

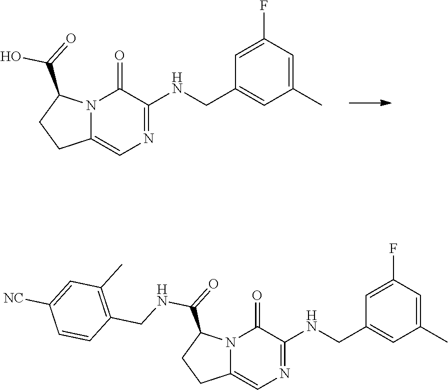 [SMILES: Cc1cc(F)cc(CNc2ncc3n(c2=O)[C@H](C(=O)O)CC3)c1 ; Cc1cc(F)cc(CNc2ncc3n(c2=O)[C@H](C(=O)NCc2ccc(C#N)cc2C)CC3)c1]

Step 1: The above compound was synthesized according to General Amide Coupling Procedure B to afford (S)—N-(4-cyano-2-methylbenzyl)-3-((3-fluoro-5-methylbenzyl)amino)-4-oxo-4,6,7,8-tetrahydropyrrolo[1,2-a]pyrazine-6-carboxamide as a white solid (32 mg, 80% yield).

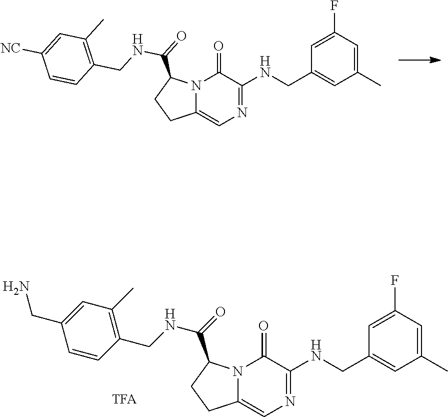 [SMILES: Cc1cc(F)cc(CNc2ncc3n(c2=O)[C@H](C(=O)NCc2ccc(C#N)cc2C)CC3)c1 ; Cc1cc(F)cc(CNc2ncc3n(c2=O)[C@H](C(=O)NCc2ccc(CN)cc2C)CC3)c1]

Step 2: To a solution of (S)—N-(4-cyano-2-methylbenzyl)-3-((3-fluoro-5-methylbenzyl)amino)-4-oxo-4,6,7,8-tetrahydropyrrolo[1,2-a]pyrazine-6-carboxamide (40 mg, 0.09 mmol) in 7 M ammonia in MeOH (1.3 mL), Raney® nickel (53 mg, 9.0 μmol) was added. The reaction mixture was evacuated and charged with Ar, then evacuated and charged with H₂ to 1 atm for 12 h at 21° C. The reaction mixture was filtered through Celite® using a mixture of MeOH and CH₂Cl₂, and was then concentrated. The filtrate was purified on a reverse phase prep column (15-50% ACN/H₂O w/0.1% TFA), concentrated, and lyophilized to afford (S)—N-(4-(aminomethyl)-2-methylbenzyl)-3-((3-fluoro-5-methylbenzyl)amino)-4-oxo-4,6,7,8-tetrahydropyrrolo[1,2-a]pyrazine-6-carboxamide trifluoroacetate (12 mg, 24% yield) as a white solid.

The following compound was prepared according to the foregoing procedures using the appropriate amine:

| | Cmp |
|---|---|
| Compound Name | No. |
| (S)-N-(4-(aminomethyl)-2,6-dimethylbenzyl)-3-((3-fluoro-5- | 206 |
| methylbenzyl)amino)-4-oxo-4,6,7,8-tetrahydropyrrolo[1,2- | |
| a]pyrazine-6-carboxamide trifluoroacetate | |

### Example 43

### Preparation of (S)—N-((6-aminopyridin-3-yl)methyl)-3-((3-isopropylbenzyl)amino)-4-oxo-4,6,7,8-tetrahydropyrrolo[1,2-a]pyrazine-6-carboxamide trifluoroacetate

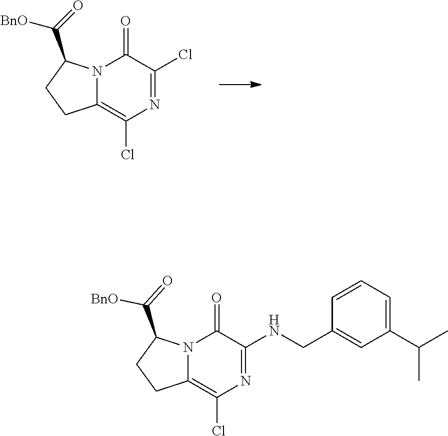 [SMILES: O=C(OCc1ccccc1)[C@@H]1CCc2c(Cl)nc(Cl)c(=O)n21 ; CC(C)c1cccc(CNc2nc(Cl)c3n(c2=O)[C@H](C(=O)OCc2ccccc2)CC3)c1]

Step 1: The above compound was synthesized according to General Chloro Displacement Procedure A to afford benzyl (S)-1-chloro-3-((3-isopropylbenzyl)amino)-4-oxo-4,6,7,8-tetrahydropyrrolo[1,2-a]pyrazine-6-carboxylate (125 mg, 94% yield) as a yellow solid.

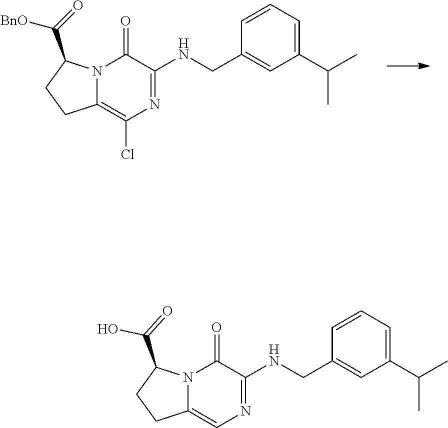 [SMILES: CC(C)c1cccc(CNc2nc(Cl)c3n(c2=O)[C@H](C(=O)OCc2ccccc2)CC3)c1 ; CC(C)c1cccc(CNc2ncc3n(c2=O)[C@H](C(=O)O)CC3)c1]

Step 2: The above compound was synthesized according to General Hydrogenolysis Procedure A to afford (S)-3-((3-isopropylbenzyl)amino)-4-oxo-4,6,7,8-tetrahydropyrrolo[1,2-a]pyrazine-6-carboxylic acid as a clear oil (quantitative yield).

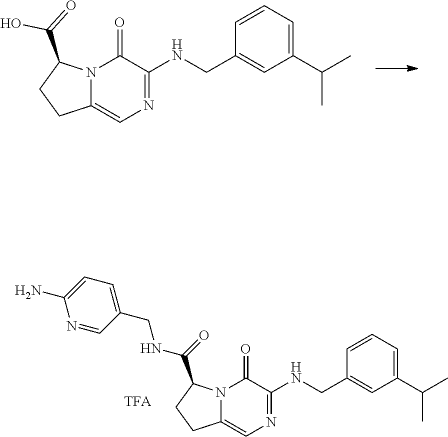 [SMILES: CC(C)c1cccc(CNc2ncc3n(c2=O)[C@H](C(=O)O)CC3)c1 ; CC(C)c1cccc(CNc2ncc3n(c2=O)[C@H](C(=O)NCc2ccc(N)nc2)CC3)c1]

Step 3: The above compound was synthesized according to General Amide Coupling Procedure A, except that prep HPLC was used during purification to afford (S)—N-((6-aminopyridin-3-yl)methyl)-3-((3-isopropylbenzyl)amino)-4-oxo-4,6,7,8-tetrahydropyrrolo[1,2-a]pyrazine-6-carboxamide trifluoroacetate as a white solid (16 mg, 32% yield).

The following compound was prepared according to the foregoing procedures using the appropriate amine:

| | Cmp |
|---|---|
| Compound Name | No. |
| ((S)-N-((6-amino-2-methylpyridin-3-yl)methyl)-3-((3- | 208 |
| isopropylbenzyl)amino)-4-oxo-4,6,7,8-tetrahydropyrrolo[1,2- | |
| a]pyrazine-6-carboxamide trifluoroacetate | |

### Example 44

### Preparation of (S)—N-((5,6-dihydro-4H-thieno[2,3-c]pyrrol-2-yl)methyl)-3-((3-fluoro-5-methylbenzyl)amino)-1-(1-methyl-1H-pyrazol-5-yl)-4-oxo-4,6,7,8-tetrahydropyrrolo[1,2-a]pyrazine-6-carboxamide trifluoroacetate

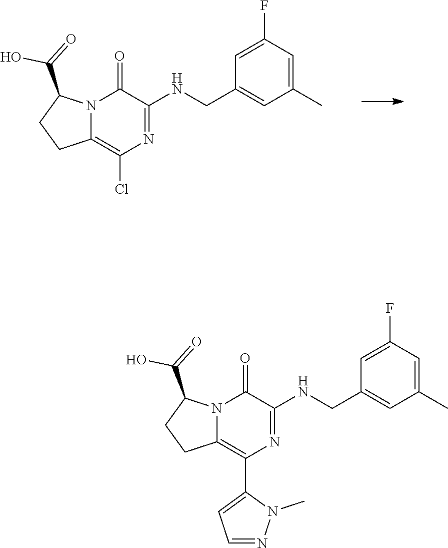 [SMILES: Cc1cc(F)cc(CNc2nc(Cl)c3n(c2=O)[C@H](C(=O)O)CC3)c1 ; Cc1cc(F)cc(CNc2nc(-c3ccnn3C)c3n(c2=O)[C@H](C(=O)O)CC3)c1]

Step 1: The above compound was synthesized according to General Suzuki-Miyaura reaction procedure A to afford (S)-3-((3-fluoro-5-methylbenzyl)amino)-1-(1-methyl-1H-pyrazol-5-yl)-4-oxo-4,6,7,8-tetrahydropyrrolo[1,2-a]pyrazine-6-carboxylic acid as a white solid (40 mg, 71% yield).

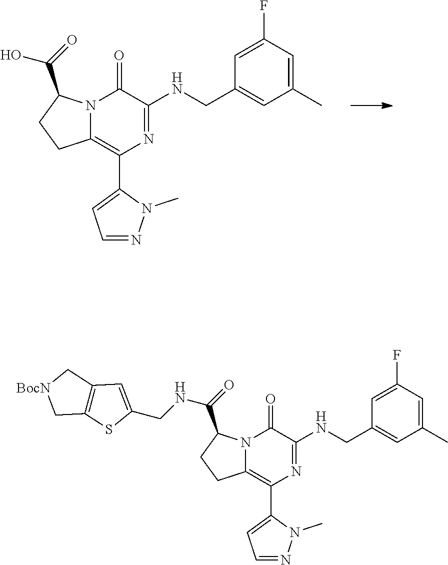 [SMILES: Cc1cc(F)cc(CNc2nc(-c3ccnn3C)c3n(c2=O)[C@H](C(=O)O)CC3)c1 ; Cc1cc(F)cc(CNc2nc(-c3ccnn3C)c3n(c2=O)[C@H](C(=O)NCc2cc4c(s2)CN(C(=O)OC(C)(C)C)C4)CC3)c1]

Step 2: The above compound was synthesized according to General Amide Coupling Procedure A to afford tert-butyl (S)-2-((3-((3-fluoro-5-methylbenzyl)amino)-1-(1-methyl-1H-pyrazol-5-yl)-4-oxo-4,6,7,8-tetrahydropyrrolo[1,2-a]pyrazine-6-carboxamido)methyl)-4,6-dihydro-5H-thieno[2,3-c]pyrrole-5-carboxylate as a sticky solid (30 mg, 94% yield).

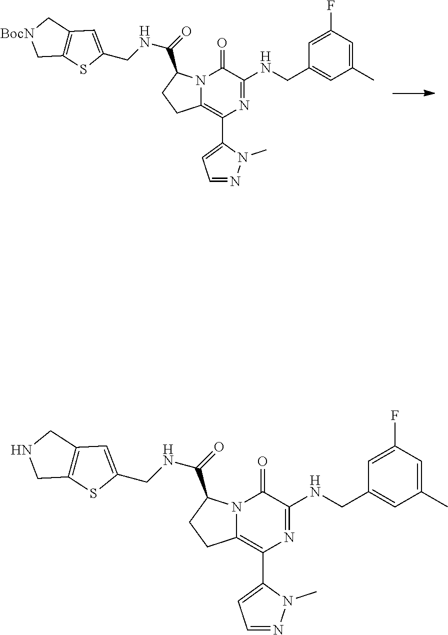 [SMILES: Cc1cc(F)cc(CNc2nc(-c3ccnn3C)c3n(c2=O)[C@H](C(=O)NCc2cc4c(s2)CN(C(=O)OC(C)(C)C)C4)CC3)c1 ; Cc1cc(F)cc(CNc2nc(-c3ccnn3C)c3n(c2=O)[C@H](C(=O)NCc2cc4c(s2)CNC4)CC3)c1]

Step 3: The above compound was synthesized according to General Boc Removal Procedure A, except that prep HPLC purification was performed to afford (S)—N-((5,6-dihydro-4H-thieno[2,3-c]pyrrol-2-yl)methyl)-3-((3-fluoro-5-methylbenzyl)amino)-1-(1-methyl-1H-pyrazol-5-yl)-4-oxo-4,6,7,8-tetrahydropyrrolo[1,2-a]pyrazine-6-carboxamide trifluoroacetate (26 mg, 85% yield).

### Example 45

### Preparation of (S)—N-((6-amino-2-methylpyridin-3-yl)methyl)-1-cyclopropyl-3-((3-fluoro-5-methylbenzyl)amino)-4-oxo-4,6,7,8-tetrahydropyrrolo[1,2-a]pyrazine-6-carboxamide trifluoroacetate

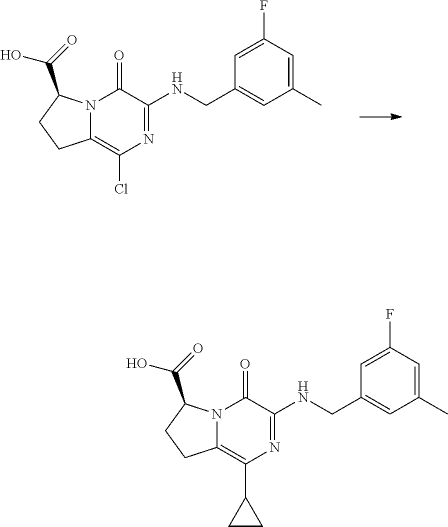 [SMILES: Cc1cc(F)cc(CNc2nc(Cl)c3n(c2=O)[C@H](C(=O)O)CC3)c1 ; Cc1cc(F)cc(CNc2nc(C3CC3)c3n(c2=O)[C@H](C(=O)O)CC3)c1]

Step 1: To a vial was added (S)-1-chloro-3-((3-fluoro-5-methylbenzyl)amino)-4-oxo-4,6,7,8-tetrahydropyrrolo[1,2-a]pyrazine-6-carboxylic acid (25 mg, 71 μmol), cyclopropylboronic acid (9.2 mg, 0.11 mmol), Cs₂CO₃ (46 mg, 0.14 mmol), mesylate[(di(1-adamantyl)-n-butylphosphine)-2-(2′-amino-1,1′-biphenyl)]palladium(II) (5.2 mg, 7.1 μmol). Toluene (1.0 mL) and water (0.1 mL) were added to the reaction mixture, and the reaction mixture was stirred at 90° C. for 18 hours. The crude mixture was acidified to pH 3 using 1 M HCl and extracted with EtOAc (3×5 mL). The combined extracts were dried over Na₂SO₄, filtered, and concentrated to afford (S)-1-cyclopropyl-3-((3-fluoro-5-methylbenzyl)amino)-4-oxo-4,6,7,8-tetrahydropyrrolo[1,2-a]pyrazine-6-carboxylic acid (20 mg, 79% yield).

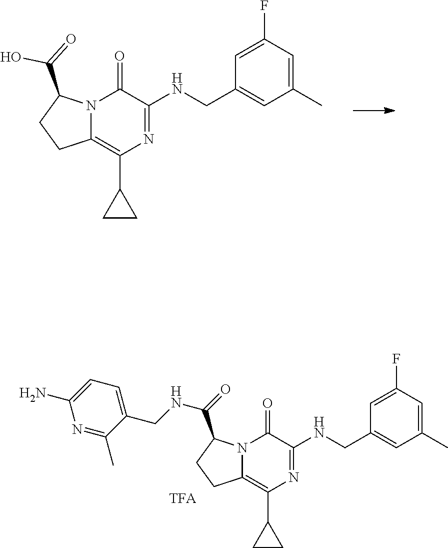 [SMILES: Cc1cc(F)cc(CNc2nc(C3CC3)c3n(c2=O)[C@H](C(=O)O)CC3)c1 ; Cc1cc(F)cc(CNc2nc(C3CC3)c3n(c2=O)[C@H](C(=O)NCc2ccc(N)nc2C)CC3)c1]

Step 2: The above compound was synthesized according to General Amide Coupling Procedure A, except that it was purified by using prep HPLC to afford (S)—N-((6-amino-2-methylpyridin-3-yl)methyl)-1-cyclopropyl-3-((3-fluoro-5-methylbenzyl)amino)-4-oxo-4,6,7,8-tetrahydropyrrolo[1,2-a]pyrazine-6-carboxamide trifluoroacetate (13 mg, 77% yield) as a white solid.

The following compound was prepared according to the foregoing procedures using the appropriate amine and boronic acid:

| | Cmp |
|---|---|
| Compound Name | No. |
| (S)-N-((6-amino-2-methylpyridin-3-yl)methyl)-3-((3-fluoro-5- | 211 |
| methylbenzyl)amino)-1-isopropyl-4-oxo-4,6,7,8-tetrahydro- | |
| pyrrolo[1,2-a]pyrazine-6-carboxamide trifluoroacetate | |

### Example 46

### Preparation of (S)—N-((6-amino-2-methylpyridin-3-yl)methyl)-1-chloro-3-((3-fluoro-5-methylbenzyl)amino)-4-oxo-4,6,7,8-tetrahydropyrrolo[1,2-a]pyrazine-6-carboxamide trifluoroacetate

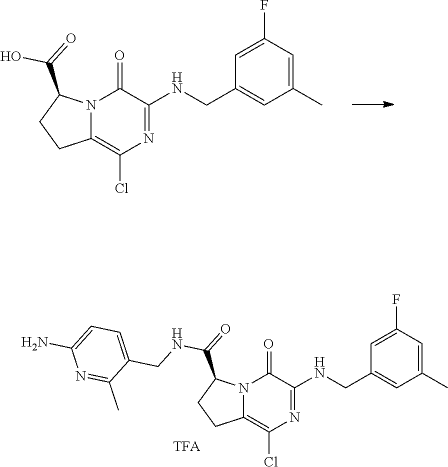 [SMILES: Cc1cc(F)cc(CNc2nc(Cl)c3n(c2=O)[C@H](C(=O)O)CC3)c1 ; Cc1cc(F)cc(CNc2nc(Cl)c3n(c2=O)[C@H](C(=O)NCc2ccc(N)nc2C)CC3)c1]

Step 1: The above compound was synthesized according to General Amide Coupling Procedure A, except that it was purified by using prep HPLC to afford (S)—N-((6-amino-2-methylpyridin-3-yl)methyl)-1-chloro-3-((3-fluoro-5-methylbenzyl)amino)-4-oxo-4,6,7,8-tetrahydropyrrolo[1,2-a]pyrazine-6-carboxamide trifluoroacetate (5 mg, 30% yield) as a white solid.

### Example 47

### Preparation of (S)—N-((6-amino-5-methylpyridin-3-yl)methyl)-3-((3-fluoro-5-methylbenzyl)amino)-4-oxo-4,6,7,8-tetrahydropyrrolo[1,2-a]pyrazine-6-carboxamide trifluoroacetate

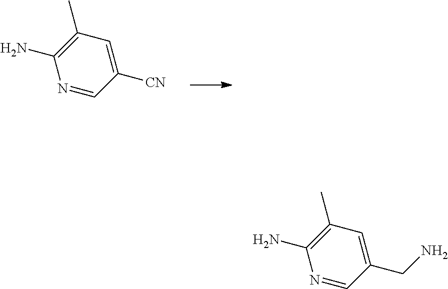

Step 1: The above compound was synthesized according to the general Raney®-Ni Hydrogenation Procedure to afford 5-(aminomethyl)-3-methylpyridin-2-amine as an oil (150 mg, 97% yield).

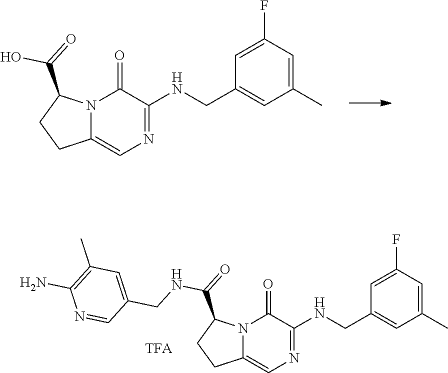 [SMILES: Cc1cc(F)cc(CNc2ncc3n(c2=O)[C@H](C(=O)O)CC3)c1 ; Cc1cc(F)cc(CNc2ncc3n(c2=O)[C@H](C(=O)NCc2cnc(N)c(C)c2)CC3)c1]

Step 2: The above compound was synthesized according to General Amide Coupling Procedure A, except that it was purified by using prep HPLC to afford (S)—N-((6-amino-5-methylpyridin-3-yl)methyl)-3-((3-fluoro-5-methylbenzyl)amino)-4-oxo-4,6,7,8-tetrahydropyrrolo[1,2-a]pyrazine-6-carboxamide trifluoroacetate (25 mg, 48% yield) as a white solid.

### Example 48

### Preparation of (S)—N-((6-amino-2-methylpyridin-3-yl)methyl)-1-(1-(difluoromethyl)-1H-pyrazol-4-yl)-3-((3-fluoro-5-methylbenzyl)amino)-4-oxo-4,6,7,8-tetrahydropyrrolo[1,2-a]pyrazine-6-carboxamide trifluoroacetate

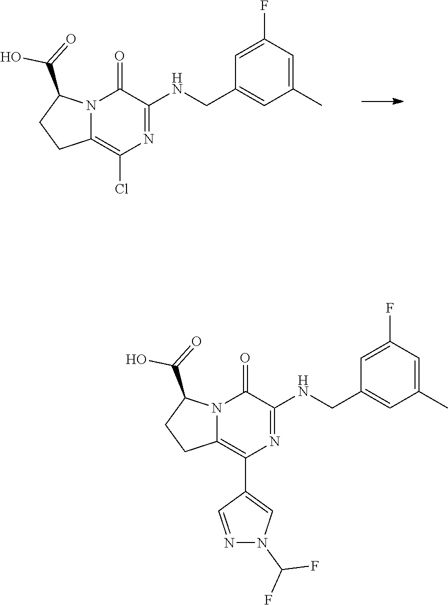 [SMILES: Cc1cc(F)cc(CNc2nc(Cl)c3n(c2=O)[C@H](C(=O)O)CC3)c1 ; Cc1cc(F)cc(CNc2nc(-c3cnn(C(F)F)c3)c3n(c2=O)[C@H](C(=O)O)CC3)c1]

Step 1: To a microwave vial was added (S)-1-chloro-3-((3-fluoro-5-methylbenzyl)amino)-4-oxo-4,6,7,8-tetrahydropyrrolo[1,2-a]pyrazine-6-carboxylic acid (70 mg, 0.20 mmol), 1-(difluoromethyl)-4-(tetramethyl-1,3,2-dioxaborolan-2-yl)-1H-pyrazole (49 mg, 0.20 mmol), K₂CO₃ (55 mg, 0.40 mmol), Pd-PEPPSI™-IPent catalyst (6.8 mg, 9.9 μmol). EtOH (1.0 mL) was then added, and the mixture was stirred at 100° C. for 30 min under microwave irradiation. The crude mixture was acidified to pH 3 using 1 M HCl and extracted with EtOAc (3×5 mL). The combined extracts were dried over Na₂SO₄ to afford (S)-1-(1-(difluoromethyl)-1H-pyrazol-4-yl)-3-((3-fluoro-5-methylbenzyl)amino)-4-oxo-4,6,7,8-tetrahydropyrrolo[1,2-a]pyrazine-6-carboxylic acid (50 mg, 58% yield).

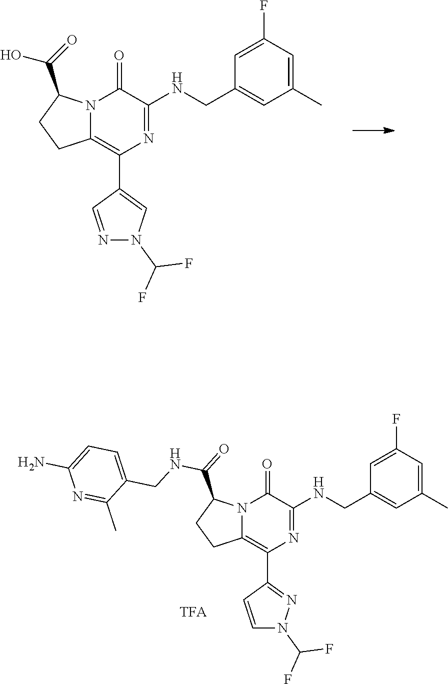 [SMILES: Cc1cc(F)cc(CNc2nc(-c3cnn(C(F)F)c3)c3n(c2=O)[C@H](C(=O)O)CC3)c1 ; Cc1cc(F)cc(CNc2nc(-c3ccn(C(F)F)n3)c3n(c2=O)[C@H](C(=O)NCc2ccc(N)nc2C)CC3)c1]

Step 2: The above compound was synthesized according to General Amide Coupling Procedure A, except that it was purified by using prep HPLC to afford (S)—N-((6-amino-2-methylpyridin-3-yl)methyl)-1-(1-(difluoromethyl)-1H-pyrazol-4-yl)-3-((3-fluoro-5-methylbenzyl)amino)-4-oxo-4,6,7,8-tetrahydropyrrolo[1,2-a]pyrazine-6-carboxamide trifluoroacetate as a white solid (8 mg, 26% yield).

The following compounds were prepared according to the foregoing procedures using the appropriate amines and boronic acids:

| | Cmp |
|---|---|
| Compound Name | No. |
| (S)-N-((1H-pyrrolo[3,2-c]pyridin-2-yl)methyl)-1-(1-(difluoro- | 215 |
| methyl)-1H-pyrazol-4-yl)-3-((3-fluoro-5-methylbenzyl)amino)-4- | |
| oxo-4,6,7,8-tetrahydropyrrolo[1,2-a]pyrazine-6-carboxamide | |
| trifluoroacetate | |
| (S)-N-((6-aminopyridin-3-yl)methyl)-1-(1-(difluoromethyl)-1H- | 216 |
| pyrazol-4-yl)-3-((3-fluoro-5-methylbenzyl)amino)-4-oxo-4,6,7,8- | |
| tetrahydropyrrolo[1,2-a]pyrazine-6-carboxamide trifluoroacetate | |
| (S)-N-((6-amino-2-methylpyridin-3-yl)methyl)-3-((3-fluoro-5- | 217 |
| methylbenzyl)amino)-4-oxo-1-phenyl-4,6,7,8-tetrahydro- | |
| pyrrolo[1,2-a]pyrazine-6-carboxamide trifluoroacetate | |
| (S)-N-((6-amino-2-methylpyridin-3-yl)methyl)-3-((3-fluoro-5- | 218 |
| methylbenzyl)amino)-1-(methyl-d3)-4-oxo-4,6,7,8-tetrahydro- | |
| pyrrolo[1,2-a]pyrazine-6-carboxamide trifluoroacetate | |

### Example 49

### Preparation of (S)-1-(1-(difluoromethyl)-1H-pyrazol-4-yl)-N-((5,6-dihydro-4H-thieno[2,3-c]pyrrol-2-yl)methyl)-3-((3-fluoro-5-methylbenzyl)amino)-4-oxo-4,6,7,8-tetrahydropyrrolo[1,2-a]pyrazine-6-carboxamide trifluoroacetate

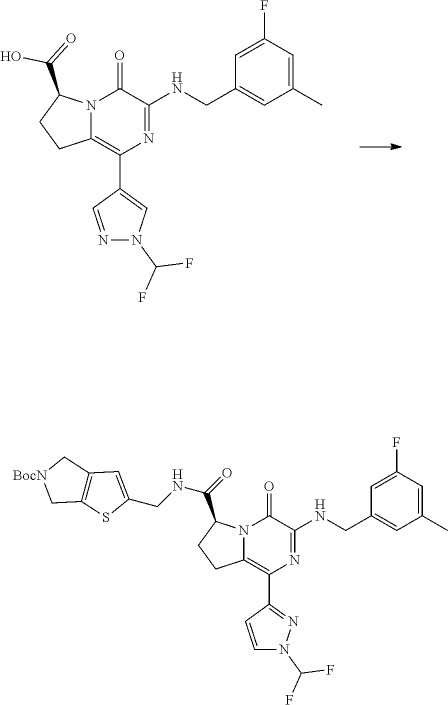 [SMILES: Cc1cc(F)cc(CNc2nc(-c3cnn(C(F)F)c3)c3n(c2=O)[C@H](C(=O)O)CC3)c1 ; Cc1cc(F)cc(CNc2nc(-c3ccn(C(F)F)n3)c3n(c2=O)[C@H](C(=O)NCc2cc4c(s2)CN(C(=O)OC(C)(C)C)C4)CC3)c1]

Step 1: The above compound was synthesized according to General Amide Coupling Procedure A to afford tert-butyl (S)-2-((1-(1-(difluoromethyl)-1H-pyrazol-4-yl)-3-((3-fluoro-5-methylbenzyl)amino)-4-oxo-4,6,7,8-tetrahydropyrrolo[1,2-a]pyrazine-6-carboxamido)methyl)-4,6-dihydro-5H-thieno[2,3-c]pyrrole-5-carboxylate as a sticky solid (30 mg, 78% yield).

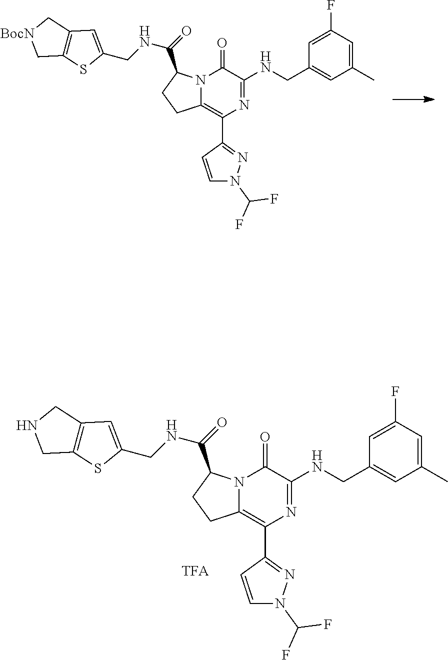 [SMILES: Cc1cc(F)cc(CNc2nc(-c3ccn(C(F)F)n3)c3n(c2=O)[C@H](C(=O)NCc2cc4c(s2)CN(C(=O)OC(C)(C)C)C4)CC3)c1 ; Cc1cc(F)cc(CNc2nc(-c3ccn(C(F)F)n3)c3n(c2=O)[C@H](C(=O)NCc2cc4c(s2)CNC4)CC3)c1]

Step 2: The above compound was synthesized according to General Boc Removal Procedure A, except that prep HPLC purification was performed to afford (S)-1-(1-(difluoromethyl)-1H-pyrazol-4-yl)-N-((5,6-dihydro-4H-thieno[2,3-c]pyrrol-2-yl)methyl)-3-((3-fluoro-5-methylbenzyl)amino)-4-oxo-4,6,7,8-tetrahydropyrrolo[1,2-a]pyrazine-6-carboxamide trifluoroacetate (8 mg, 30% yield).

### Example 50

### Preparation of(S)-3-(([1,1′-biphenyl]-3-ylmethyl)amino)-N-((6-amino-2-methylpyridin-3-yl)methyl)-4-oxo-4,6,7,8-tetrahydropyrrolo[1,2-a]pyrazine-6-carboxamide trifluoroacetate and (S)-3-(([1,1′-biphenyl]-3-ylmethyl)amino)-N-((6-amino-2-methylpyridin-3-yl)methyl)-1-chloro-4-oxo-4,6,7,8-tetrahydropyrrolo[1,2-a]pyrazine-6-carboxamide trifluoroacetate

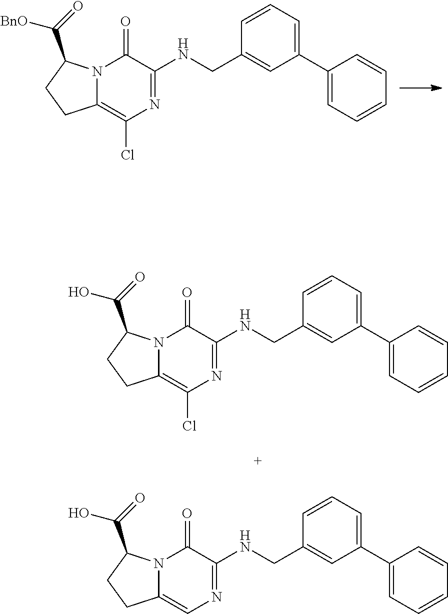 [SMILES: O=C(OCc1ccccc1)[C@@H]1CCc2c(Cl)nc(NCc3cccc(-c4ccccc4)c3)c(=O)n21 ; O=C(O)[C@@H]1CCc2c(Cl)nc(NCc3cccc(-c4ccccc4)c3)c(=O)n21 ; O=C(O)[C@@H]1CCc2cnc(NCc3cccc(-c4ccccc4)c3)c(=O)n21]

Step 1: The above compound was synthesized according to General Hydrogenolysis Procedure A to afford (S)-3-(([1,1′-biphenyl]-3-ylmethyl)amino)-4-oxo-4,6,7,8-tetrahydropyrrolo[1,2-a]pyrazine-6-carboxylic acid (40 mg, 60% yield) and (S)-3-(([1,1′-biphenyl]-3-ylmethyl)amino)-1-chloro-4-oxo-4,6,7,8-tetrahydropyrrolo[1,2-a]pyrazine-6-carboxylic acid (20 mg, 27% yield) as semi-solids.

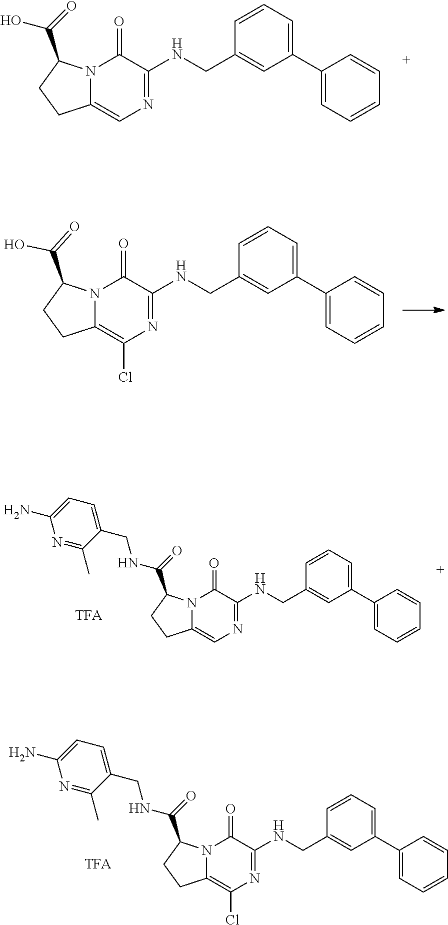 [SMILES: O=C(O)[C@@H]1CCc2cnc(NCc3cccc(-c4ccccc4)c3)c(=O)n21 ; O=C(O)[C@@H]1CCc2c(Cl)nc(NCc3cccc(-c4ccccc4)c3)c(=O)n21 ; Cc1nc(N)ccc1CNC(=O)[C@@H]1CCc2cnc(NCc3cccc(-c4ccccc4)c3)c(=O)n21 ; Cc1nc(N)ccc1CNC(=O)[C@@H]1CCc2c(Cl)nc(NCc3cccc(-c4ccccc4)c3)c(=O)n21]

Step 2: The above compound was synthesized according to General Amide Coupling Procedure A, except that the material was purified by prep HPLC to afford (S)-3-(([1,1′-biphenyl]-3-ylmethyl)amino)-N-((6-amino-2-methylpyridin-3-yl)methyl)-4-oxo-4,6,7,8-tetrahydropyrrolo[1,2-a]pyrazine-6-carboxamide trifluoroacetate (28 mg, 43% yield) and (S)-3-(([1,1′-biphenyl]-3-ylmethyl)amino)-N-((6-amino-2-methylpyridin-3-yl)methyl)-1-chloro-4-oxo-4,6,7,8-tetrahydropyrrolo[1,2-a]pyrazine-6-carboxamide trifluoroacetate (12 mg, 17% yield) as white solids.

### Example 51

### Preparation of (S)—N-((6-amino-2-methylpyridin-3-yl)methyl)-1-cyano-3-((3-fluoro-5-methylbenzyl)amino)-4-oxo-4,6,7,8-tetrahydropyrrolo[1,2-a]pyrazine-6-carboxamide trifluoroacetate

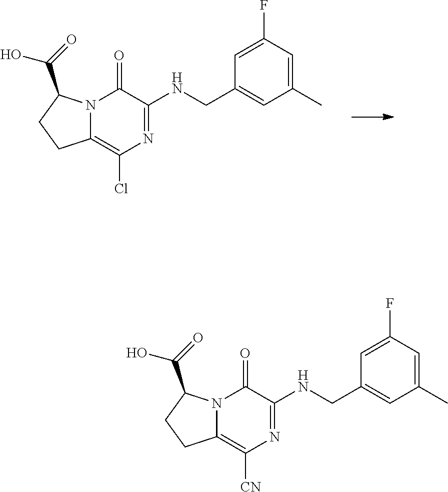 [SMILES: Cc1cc(F)cc(CNc2nc(Cl)c3n(c2=O)[C@H](C(=O)O)CC3)c1 ; Cc1cc(F)cc(CNc2nc(C#N)c3n(c2=O)[C@H](C(=O)O)CC3)c1]

Step 1: To a 50 mL round bottomed flask was added (S)-1-chloro-3-((3-fluoro-5-methylbenzyl)amino)-4-oxo-4,6,7,8-tetrahydropyrrolo[1,2-a]pyrazine-6-carboxylic acid (60 mg, 0.17 mmol), zinc (11 mg, 0.17 mmol), zinc cyanide (20 mg, 11 μL, 0.17 mmol), and Pd TrixiePhos G3 (1.3 mg, 1.7 μmol). NMP (0.5 mL) was added and the mixture was purged with Ar. The reaction mixture was stirred at 110° C. for 16 h, then filtered and concentrated to afford (S)-1-cyano-3-((3-fluoro-5-methylbenzyl)amino)-4-oxo-4,6,7,8-tetrahydropyrrolo[1,2-a]pyrazine-6-carboxylic acid (15 mg, 26% yield).

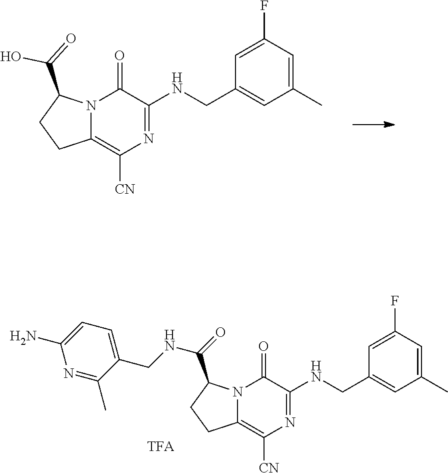 [SMILES: Cc1cc(F)cc(CNc2nc(C#N)c3n(c2=O)[C@H](C(=O)O)CC3)c1 ; Cc1cc(F)cc(CNc2nc(C#N)c3n(c2=O)[C@H](C(=O)NCc2ccc(N)nc2C)CC3)c1]

Step 2: The above compound was synthesized according to General Amide Coupling Procedure A, except that it was purified by using prep HPLC to afford (S)—N-((6-amino-2-methylpyridin-3-yl)methyl)-1-cyano-3-((3-fluoro-5-methylbenzyl)amino)-4-oxo-4,6,7,8-tetrahydropyrrolo[1,2-a]pyrazine-6-carboxamide trifluoroacetate (19 mg, 75% yield) as a white solid.

The following compound was prepared according to the foregoing procedures using the appropriate amine and boronic acid:

| | Cmp |
|---|---|
| Compound Name | No. |
| (S)-N-((6-amino-2-methylpyridin-3-yl)methyl)-1-cyano-3-((2- | 223 |
| fluoro-5-methylbenzyl)amino)-4-oxo-4,6,7,8-tetrahydropyrrolo[1,2- | |
| a]pyrazine-6-carboxamide trifluoroacetate | |

### Example 52

### Preparation of (S)—N—((R)-1-(6-aminopyridin-3-yl)ethyl)-3-((2,3-difluoro-5-methylbenzyl)amino)-4-oxo-4,6,7,8-tetrahydropyrrolo[1,2-a]pyrazine-6-carboxamide trifluoroacetate

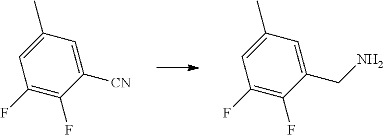

Step 1: The above compound was synthesized according to the General Raney®-Ni Hydrogenation procedure to afford (2,3-difluoro-5-methylphenyl)methanamine as an oil (450 mg, 99% yield).

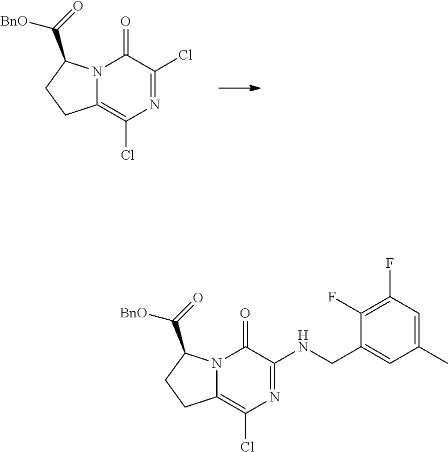 [SMILES: O=C(OCc1ccccc1)[C@@H]1CCc2c(Cl)nc(Cl)c(=O)n21 ; Cc1cc(F)c(F)c(CNc2nc(Cl)c3n(c2=O)[C@H](C(=O)OCc2ccccc2)CC3)c1]

Step 2: The above compound was synthesized according to General Chloro Displacement Procedure A to afford benzyl (S)-1-chloro-3-((2,3-difluoro-5-methylbenzyl)amino)-4-oxo-4,6,7,8-tetrahydropyrrolo[1,2-a]pyrazine-6-carboxylate (130 mg, 87% yield) as a semi-solid.

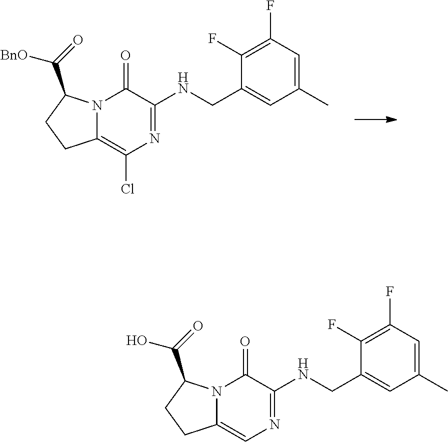 [SMILES: Cc1cc(F)c(F)c(CNc2nc(Cl)c3n(c2=O)[C@H](C(=O)OCc2ccccc2)CC3)c1 ; Cc1cc(F)c(F)c(CNc2ncc3n(c2=O)[C@H](C(=O)O)CC3)c1]

Step 3: The above compound was synthesized according to General Hydrogenolysis Procedure A to afford (S)-3-((2,3-difluoro-5-methylbenzyl)amino)-4-oxo-4,6,7,8-tetrahydropyrrolo[1,2-a]pyrazine-6-carboxylic acid (90 mg, 95% yield).

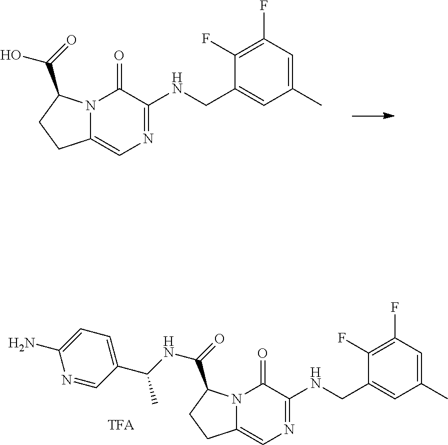 [SMILES: Cc1cc(F)c(F)c(CNc2ncc3n(c2=O)[C@H](C(=O)O)CC3)c1 ; Cc1cc(F)c(F)c(CNc2ncc3n(c2=O)[C@H](C(=O)N[C@H](C)c2ccc(N)nc2)CC3)c1]

Step 4: The above compound was synthesized according to General Amide Coupling Procedure A, except that it was purified by using prep HPLC to afford (S)—N—((R)-1-(6-aminopyridin-3-yl)ethyl)-3-((2,3-difluoro-5-methylbenzyl)amino)-4-oxo-4,6,7,8-tetrahydropyrrolo[1,2-a]pyrazine-6-carboxamide trifluoroacetate (24 mg, 28% yield) as a white solid.

### Example 53

### Preparation of (S)—N-((5-(aminomethyl)thiophen-2-yl)methyl)-1-chloro-4-oxo-3-(((5,6,7,8-tetrahydronaphthalen-2-yl)methyl)amino)-4,6,7,8-tetrahydropyrrolo[1,2-a]pyrazine-6-carboxamide trifluoroacetate

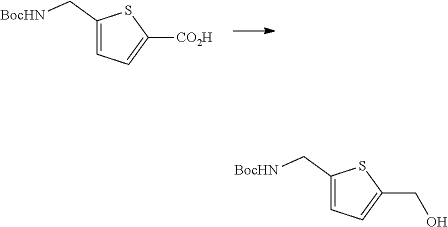 [SMILES: CC(C)(C)OC(=O)NCc1ccc(C(=O)O)s1 ; CC(C)(C)OC(=O)NCc1ccc(CO)s1]

Step 1: A mixture of 5-(((tert-butoxycarbonyl)amino)methyl)thiophene-2-carboxylic acid (514 mg, 2 mmol) in THF (42 mL) was put under N₂ and cooled to 0° C. A solution of borane THF complex (8 mmol) was added dropwise. The reaction was stirred at 0° C. for 5 min and warmed to room temp while stirring for 16 h. The reaction was quenched with anhydrous methanol at 0° C., then stirred at room temp for 1 h. Volatiles were evaporated under reduced pressure. The crude product was dissolved in CH₂Cl₂ and adsorbed onto silica gel. Purification by chromatography (0-100% EtOAc-hexane) afforded tert-butyl ((5-(hydroxymethyl)thiophen-2-yl)methyl)carbamate (365 mg, 75% yield) as a light yellow solid.

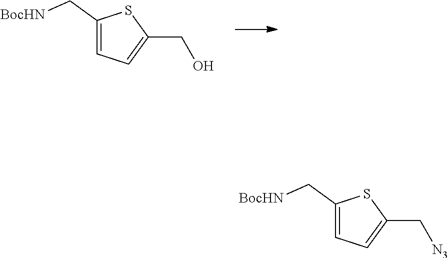 [SMILES: CC(C)(C)OC(=O)NCc1ccc(CO)s1 ; CC(C)(C)OC(=O)NCc1ccc(CN=[N+]=[N-])s1]

Step 2: A suspension of tert-butyl ((5-(hydroxymethyl)thiophen-2-yl)methyl)carbamate (365 mg, 1.40 mmol) in THF (3.5 mL) was put under N₂ and treated with diphenylphosphoryl azide (320 mg, 2.1 mmol) at room temp. After cooling to 0° C., 1,8-diazabicyclo[5.4.0]undec-7-ene (320 mg, 2.1 mmol) was added. The reaction flask was sealed and warmed to room temp for about 5 min. The resulting solution was heated at 65° C. for 16 h. The reaction was quenched with water and extracted with EtOAc 3×. The organic layers were combined, washed with brine, and dried over anhydrous Na₂SO₄. The crude product was dissolved in CH₂Cl₂ and adsorbed onto silica gel. Purification by column chromatography (0-40% EtOAc-heptane) afforded tert-butyl ((5-(azidomethyl)thiophen-2-yl)methyl)carbamate (364 mg, 97% yield) as a white solid.

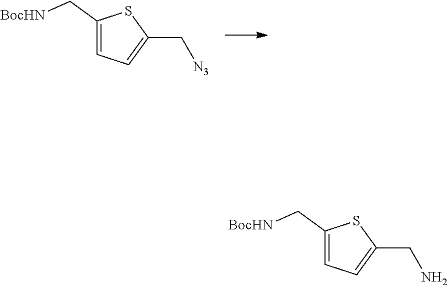 [SMILES: CC(C)(C)OC(=O)NCc1ccc(CN=[N+]=[N-])s1 ; CC(C)(C)OC(=O)NCc1ccc(CN)s1]

Step 3: To a solution of tert-butyl ((5-(azidomethyl)thiophen-2-yl)methyl)carbamate (40 mg, 0.15 mmol) in THF (0.6 mL) and water (0.075 mL) was added triphenylphosphine (43 mg, 0.16 mmol) at 21° C. The reaction mixture was stirred at 21° C. for 16 h. The solvents were removed under pressure to afford tert-butyl ((5-(aminomethyl)thiophen-2-yl)methyl)carbamate (33 mg, 0.14 mmol, 91% yield), which was used directly in the next step without further purification.

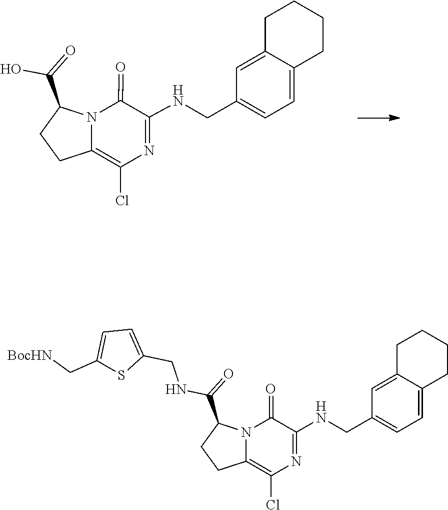 [SMILES: O=C(O)[C@@H]1CCc2c(Cl)nc(NCc3ccc4c(c3)CCCC4)c(=O)n21 ; CC(C)(C)OC(=O)NCc1ccc(CNC(=O)[C@@H]2CCc3c(Cl)nc(NCc4ccc5c(c4)CCCC5)c(=O)n32)s1]

Step 4: To a solution of (S)-1-chloro-4-oxo-3-(((5,6,7,8-tetrahydronaphthalen-2-yl)methyl)amino)-4,6,7,8-tetrahydropyrrolo[1,2-a]pyrazine-6-carboxylic acid (42 mg, 0.11 mmol, described above) in DMF (1 mL) was added NHS (14 mg, 0.12 mmol) and DCC (25 mg, 12 mmol) at 21° C. The reaction mixture was stirred for 15 min at 21° C. tert-butyl ((5-(aminomethyl)thiophen-2-yl)methyl)carbamate (30 mg, 0.12 mmol) was then added, and the reaction mixture was stirred for 1 h at 21° C. The crude reaction mixture was filtered through Celite© and concentrated to afford tert-butyl (S)-((5-((1-chloro-4-oxo-3-(((5,6,7,8-tetrahydronaphthalen-2-yl)methyl)amino)-4,6,7,8-tetrahydropyrrolo[1,2-a]pyrazine-6-carboxamido)methyl)thiophen-2-yl)methyl)carbamate (60-mg, 89% yield).

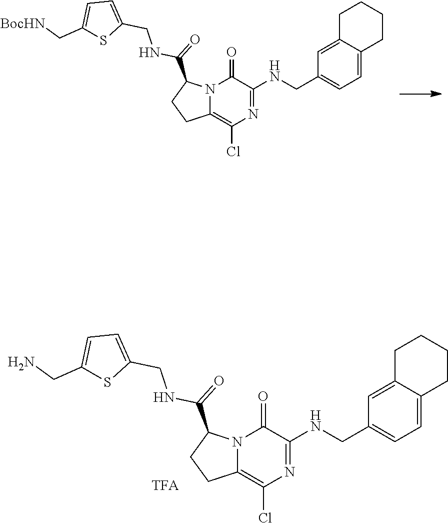 [SMILES: CC(C)(C)OC(=O)NCc1ccc(CNC(=O)[C@@H]2CCc3c(Cl)nc(NCc4ccc5c(c4)CCCC5)c(=O)n32)s1 ; NCc1ccc(CNC(=O)[C@@H]2CCc3c(Cl)nc(NCc4ccc5c(c4)CCCC5)c(=O)n32)s1]

Step 5: The above compound was synthesized according to General Boc Removal Procedure A, except that prep HPLC purification was performed to afford (S)—N-((5-(aminomethyl)thiophen-2-yl)methyl)-1-chloro-4-oxo-3-(((5,6,7,8-tetrahydronaphthalen-2-yl)methyl)amino)-4,6,7,8-tetrahydropyrrolo[1,2-a]pyrazine-6-carboxamide trifluoroacetate (22 mg, 36% yield).

### Example 54

### Preparation of (S)—N-((6-amino-2-(methyl-d₃)pyridin-3-yl)methyl)-3-((3-methoxy-5-methylbenzyl)amino)-4-oxo-4,6,7,8-tetrahydropyrrolo[1,2-a]pyrazine-6-carboxamide

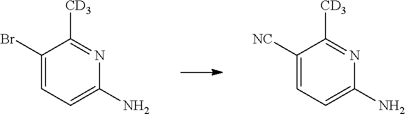 [SMILES: [2H]C([2H])([2H])c1nc(N)ccc1Br ; [2H]C([2H])([2H])c1nc(N)ccc1C#N]

Step 1: A mixture of 5-bromo-6-(methyl-d₃)pyridine-2-amine (155 mg, 0.82 mmol) and CuCN (146 mg, 1.63 mmol) in DMF (4 mL) was heated to reflux for 4 h. Upon completion, DMF was evaporated in vacuo and the residue was partitioned between EtOAc and 10% NaCN solution. The organic layer was washed with 10% NaCN solution and brine, dried over Na₂SO₄, filtered, and concentrated. The residue was purified by column chromatography (0-20% MeOH-DCM) to afford 6-amino-2-(methyl-d₃)nicotinonitrile (95 mg, 86% yield).

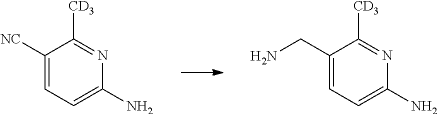 [SMILES: [2H]C([2H])([2H])c1nc(N)ccc1C#N ; [2H]C([2H])([2H])c1nc(N)ccc1CN]

Step 2: A mixture of 6-amino-2-(methyl-d₃)nicotinonitrile (95 mg, 0.7 mmol) and 10% Pd/C (74 mg, 0.07 mmol) in EtOH (2.6 mL), MeOH (1.0 mL), conc HCl, and H₂O (0.33 mL) was stirred at 60 psi for 25 h. The reaction mixture was filtered through Celite© rinsing with a solution of EtOH:MeOH (1:1), and the solvent evaporated in vacuo. The residue was purified by column chromatography (amine column, 0-30% MeOH-DCM) to provide 5-(aminomethyl)-6-(methyl-d₃)pyridin-2-amine (85 mg, 87% yield).

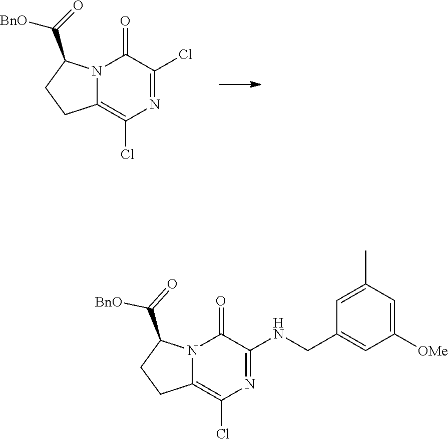 [SMILES: O=C(OCc1ccccc1)[C@@H]1CCc2c(Cl)nc(Cl)c(=O)n21 ; COc1cc(C)cc(CNc2nc(Cl)c3n(c2=O)[C@H](C(=O)OCc2ccccc2)CC3)c1]

Step 3: The above compound was synthesized according to General Chloro Displacement Procedure A to afford benzyl (S)-1-chloro-3-((3-methoxy-5-methylbenzyl)amino)-4-oxo-4,6,7,8-tetrahydropyrrolo[1,2-a]pyrazine-6-carboxylate (268 mg, 67% yield).

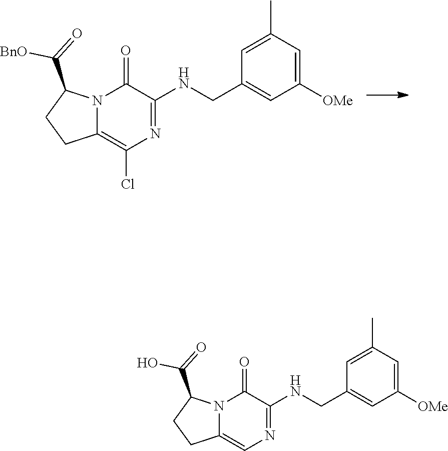 [SMILES: COc1cc(C)cc(CNc2nc(Cl)c3n(c2=O)[C@H](C(=O)OCc2ccccc2)CC3)c1 ; COc1cc(C)cc(CNc2ncc3n(c2=O)[C@H](C(=O)O)CC3)c1]

Step 4: The above compound was synthesized according to General Hydrogenolysis Procedure A to afford (S)-3-((3-methoxy-5-methylbenzyl)amino)-4-oxo-4,6,7,8-tetrahydropyrrolo[1,2-a]pyrazine-6-carboxylic acid (quantitative yield).

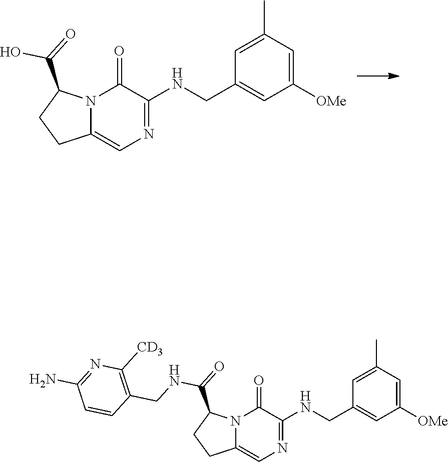 [SMILES: COc1cc(C)cc(CNc2ncc3n(c2=O)[C@H](C(=O)O)CC3)c1 ; [2H]C([2H])([2H])c1nc(N)ccc1CNC(=O)[C@@H]1CCc2cnc(NCc3cc(C)cc(OC)c3)c(=O)n21]

Step 5: To a solution of (S)-3-((3-methoxy-5-methylbenzyl)amino)-4-oxo-4,6,7,8-tetrahydropyrrolo[1,2-a]pyrazine-6-carboxylic acid (50 mg, 0.15 mmol) in DMF (2 mL) was added 5-(aminomethyl)-6-(methyl-d₃)pyridin-2-amine (26 mg, 0.19 mmol), HATU (0.17 g, 0.46 mmol), and DIEA (0.13 mL, 0.76 mmol). After stirring for 18 h, the reaction mixture was concentrated and the residue was purified by column chromatography (amine, 0-40% MeOH-DCM), and re-purified by column chromatography (0-20% MeOH-DCM) to afford (S)—N-((6-amino-2-(methyl-d₃)pyridin-3-yl)methyl)-3-((3-methoxy-5-methylbenzyl)amino)-4-oxo-4,6,7,8-tetrahydropyrrolo[1,2-a]pyrazine-6-carboxamide (19.5 mg, 28% yield).

### Example 55

### Preparation of (S)—N-((1H-pyrrolo[3,2-c]pyridin-2-yl)methyl)-3-(benzylamino)-8,8-dimethyl-4-oxo-4,6,7,8-tetrahydropyrrolo[1,2-a]pyrazine-6-carboxamide trifluoroacetate, and (6S,8R)—N-((1H-pyrrolo[3,2-c]pyridin-2-yl)methyl)-3-(benzylamino)-8-methyl-4-oxo-4,6,7,8-tetrahydropyrrolo[1,2-a]pyrazine-6-carboxamide trifluoroacetate

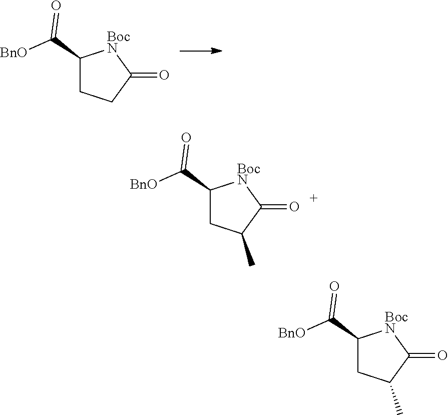 [SMILES: CC(C)(C)OC(=O)N1C(=O)CC[C@H]1C(=O)OCc1ccccc1 ; C[C@H]1C[C@@H](C(=O)OCc2ccccc2)N(C(=O)OC(C)(C)C)C1=O ; C[C@@H]1C[C@@H](C(=O)OCc2ccccc2)N(C(=O)OC(C)(C)C)C1=O]

Step 1: To a solution of 2-benzyl 1-(tert-butyl) (S)-5-oxopyrrolidine-1,2-dicarboxylate (10 g, 31 mmol) in THF (100 mL) cooled to −78° C. was added 1M LiHMDS in THF (38 mL). The mixture was stirred for 30 min, at which time Mel (2.3 mL, 38 mmol) was added. After an additional 2 h of stirring, the reaction mixture was quenched with aq. NH₄Cl solution and warmed to room temperature. The solution was extracted with EtOAc 3×. The combined organics were collected, washed with brine, and dried with Na₂SO₄. The material was partially purified by silica gel column chromatography (heptane-EtOAc gradient) to afford 2-benzyl 1-(tert-butyl) (2S,4S)-4-methyl-5-oxopyrrolidine-1,2-dicarboxylate (2.14 g, 21%), 2-benzyl 1-(tert-butyl) (2S,4R)-4-methyl-5-oxopyrrolidine-1,2-dicarboxylate (1.61 g, 15%), and a mixture of mono-alkylated cis and trans 2-benzyl 1-(tert-butyl) (2S)-4-methyl-5-oxopyrrolidine-1,2-dicarboxylate (2.47 g, 24% yield).

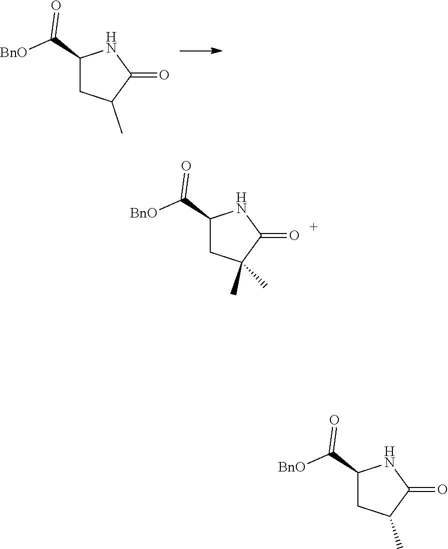

Step 2: To a solution of cis and trans 2-benzyl 1-(tert-butyl) (2S)-4-methyl-5-oxopyrrolidine-1,2-dicarboxylate (2.47 g, 7.5 mmol) in THF (25 mL) cooled to −78° C. was added 1 M LiHMDS in THF (8.2 mL). The mixture was allowed to stir for 30 min at which time Mel (1.2 g, 8.2 mmol) was added. After an additional 2 h of stirring, the reaction mixture was quenched with aqueous NH₄Cl and warmed to room temperature. The solution was extracted with EtOAc three times. The combined organics were collected, washed with brine, and dried with Na₂SO₄, filtered, and concentrated. The material was purified by silica gel column chromatography eluting with a heptane-EtOAc gradient to provide a 1:1 ratio of 2-benzyl 1-(tert-butyl) (S)-4,4-dimethyl-5-oxopyrrolidine-1,2-dicarboxylate and 2-benzyl 1-(tert-butyl) (2S,4R)-4-methyl-5-oxopyrrolidine-1,2-dicarboxylate (combined 1.35 g, 52% yield).

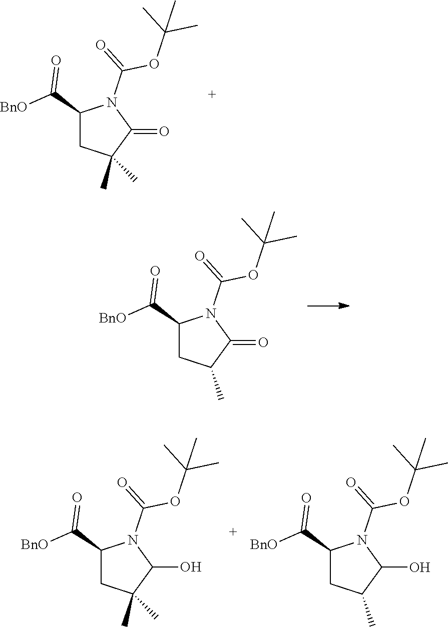

Step 3: To a 1:1 solution of 2-benzyl 1-(tert-butyl) (2S,4R)-4-methyl-5-oxopyrrolidine-1,2-dicarboxylate and 2-benzyl 1-(tert-butyl) (S)-4,4-dimethyl-5-oxopyrrolidine-1,2-dicarboxylate (1.67 g, 5.01 mmol) in THF (15 mL) at −78° C. was added 1 M lithium triethylborohydride (5.51 mL) slowly under Ar. After 30 min, the reaction was quenched with sat. aq. NaHCO₃ and warmed to 0° C. 30% aq. H₂O₂ (˜25 drops) was added and the reaction was stirred for 30 min. The organics were removed in vacuo and the aqueous solution was extracted with EtOAc 3×. The combined organics were washed with brine and dried over Na₂SO₄ to afford a 1:1 mixture of 2-benzyl 1-(tert-butyl) (2S,4R)-5-hydroxy-4-methylpyrrolidine-1,2-dicarboxylate and 2-benzyl 1-(tert-butyl) (2S)-5-hydroxy-4,4-dimethylpyrrolidine-1,2-dicarboxylate as a colorless oil (quantitative yield).

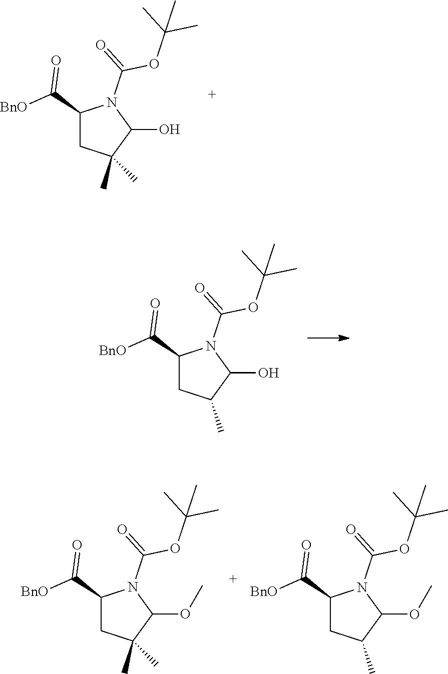

Step 4: A 1:1 mixture of 2-benzyl 1-(tert-butyl) (2S,4R)-5-hydroxy-4-methylpyrrolidine-1,2-dicarboxylate and 2-benzyl 1-(tert-butyl) (2S)-5-hydroxy-4,4-dimethylpyrrolidine-1,2-dicarboxylate (1.75 g, 5.01 mmol) was added to a round bottom flask. Dry MeOH (25 mL) and p-toluenesulfonic acid monohydrate (95 mg, 0.501 mmol) were added and the mixture was stirred overnight. The crude mixture was concentrated and resuspended in EtOAc. The organics were washed with sat. NaHCO₃ and brine, and dried over Na₂SO₄. The organic solution was concentrated to afford a mixture of (2-benzyl 1-(tert-butyl) (2S,4R)-5-methoxy-4-methylpyrrolidine-1,2-dicarboxylate and 2-benzyl 1-(tert-butyl) (2S)-5-methoxy-4,4-dimethylpyrrolidine-1,2-dicarboxylate) (1.42 g, 81% yield).

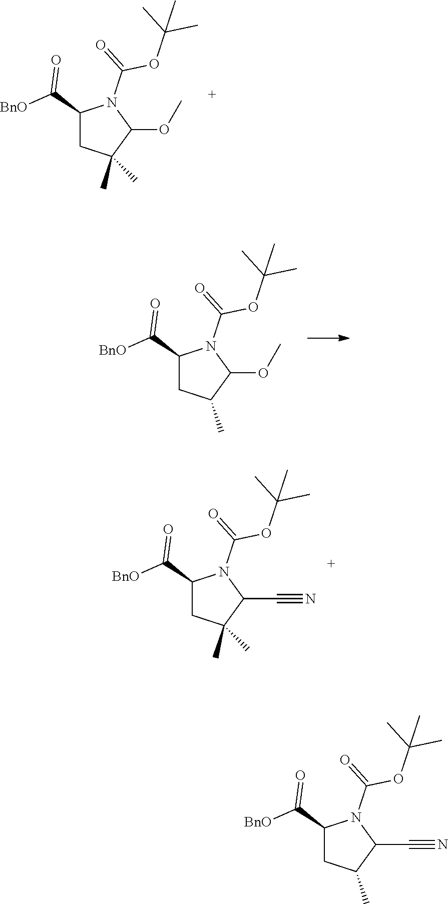

Step 5: A solution of 2-benzyl 1-(tert-butyl) (2S)-5-methoxy-4,4-dimethylpyrrolidine-1,2-dicarboxylate and 2-benzyl 1-(tert-butyl) (2S,4R)-5-methoxy-4-methylpyrrolidine-1,2-dicarboxylate (1.42 g, 4.07 mmol) in DCM (10 mL) was cooled to −78° C. TMSCN (1.212 g, 12.22 mmol) and BF₃—OEt₂ (1.734 g, 12.22 mmol) were added and the reaction was stirred for 2 h. Saturated aq. NaHCO₃ was added and the aqueous layer was washed with DCM. The organic layers were combined and concentrated. The mixture was purified by column chromatography, eluting with heptane-EtOAc, to afford a mixture of 2-benzyl 1-(tert-butyl) (2S)-5-cyano-4,4-dimethylpyrrolidine-1,2-dicarboxylate and 2-benzyl 1-(tert-butyl) (2S,4R)-5-cyano-4-methylpyrrolidine-1,2-dicarboxylate (0.99 g, 36% yield).

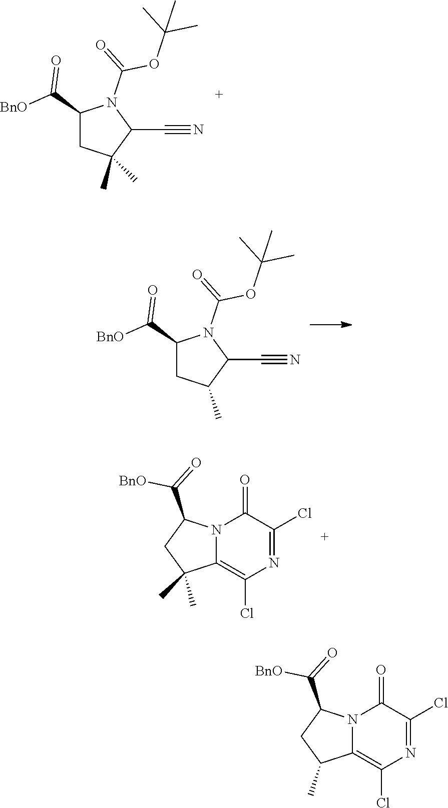

Step 6: A mixture of 2-benzyl 1-(tert-butyl) (2S)-5-cyano-4,4-dimethylpyrrolidine-1,2-dicarboxylate and 2-benzyl 1-(tert-butyl) (2S,4R)-5-cyano-4-methylpyrrolidine-1,2-dicarboxylate (0.99 g, 2.9 mmol) was dissolved in DME (30 mL) and 4 M HCl in dioxane (7.2 mL), and cooled to 0° C. The mixture was stirred 1 hour, at which time oxalyl chloride (1.5 g, 11 mmol) was added and the reaction was stirred for an additional 3 d. 1 drop of DMF was added and the reaction was stirred for 1 h. The mixture was concentrated and purified by column chromatography with heptane-EtOAc to afford a mixture of benzyl (6S,8R)-1,3-dichloro-8-methyl-4-oxo-4,6,7,8-tetrahydropyrrolo[1,2-a]pyrazine-6-carboxylate and benzyl (S)-1,3-dichloro-8,8-dimethyl-4-oxo-4,6,7,8-tetrahydropyrrolo[1,2-a]pyrazine-6-carboxylate (493 mg, 49% yield).

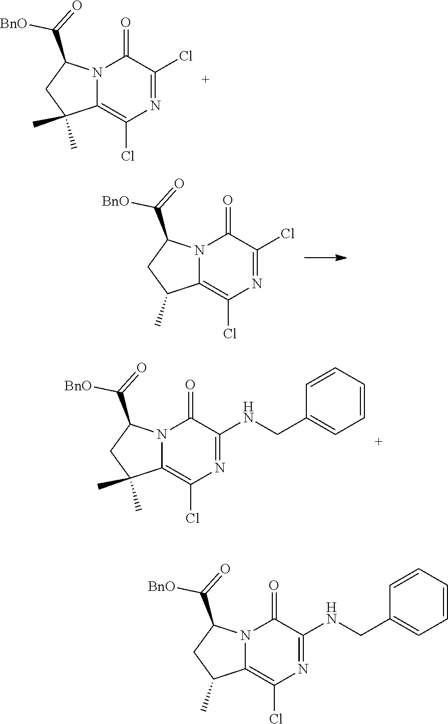

Step 7: To a 20 mL pressure vial was added a mixture of benzyl (6S,8R)-1,3-dichloro-8-methyl-4-oxo-4,6,7,8-tetrahydropyrrolo[1,2-a]pyrazine-6-carboxylate (145 mg, 411 μmol) and (S)-1,3-dichloro-8,8-dimethyl-4-oxo-4,6,7,8-tetrahydropyrrolo[1,2-a]pyrazine-6-carboxylate (151 mg, 411 μmol) and MeCN. The reaction mixture was treated with DIEA (159 mg, 1.23 mmol) and stirred at 90° C. for 3 h. The crude material was concentrated then purified by column chromatography (EtOAc-heptane) to afford benzyl (S)-3-(benzylamino)-1-chloro-8,8-dimethyl-4-oxo-4,6,7,8-tetrahydropyrrolo[1,2-a]pyrazine-6-carboxylate and benzyl (6S,8R)-3-(benzylamino)-1-chloro-8-methyl-4-oxo-4,6,7,8-tetrahydropyrrolo[1,2-a]pyrazine-6-carboxylate (162 mg, 47% yield).

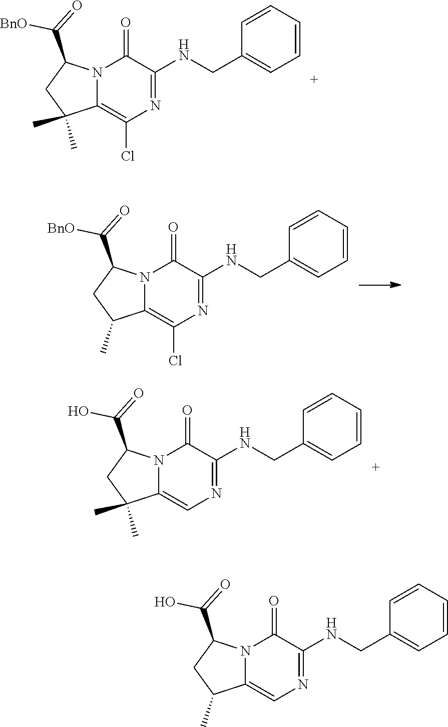

Step 8: A 100 mL round bottom flask was charged with benzyl (S)-3-(benzylamino)-1-chloro-8,8-dimethyl-4-oxo-4,6,7,8-tetrahydropyrrolo[1,2-a]pyrazine-6-carboxylate and benzyl (6S,8R)-3-(benzylamino)-1-chloro-8-methyl-4-oxo-4,6,7,8-tetrahydropyrrolo[1,2-a]pyrazine-6-carboxylate (162 mg, 382 μmol), EtOH (2 mL) and EtOAc (2 mL). The flask was purged with Ar, then 10% Pd/C (40.7 mg, 38.2 μmol) was added. The reaction mixture was then put under vacuum and backfilled with 1 atm H₂ gas three times and left to stir at 25° C. overnight under 1 atm H₂. Approximately half of the crude reaction mixture was removed for another use. Three drops of 7 N NH₃ in MeOH were added and the reaction mixture continued to stir for 2 d. The crude material was filtered through a 0.2 μm PTFE microfilter and concentrated down. The material was purified by column chromatography, eluting with DCM-MeOH to afford a mixture of (S)-3-(benzylamino)-8,8-dimethyl-4-oxo-4,6,7,8-tetrahydropyrrolo[1,2-a]pyrazine-6-carboxylic acid and (6S,8R)-3-(benzylamino)-8-methyl-4-oxo-4,6,7,8-tetrahydropyrrolo[1,2-a]pyrazine-6-carboxylic acid (65 mg, quant. yield).

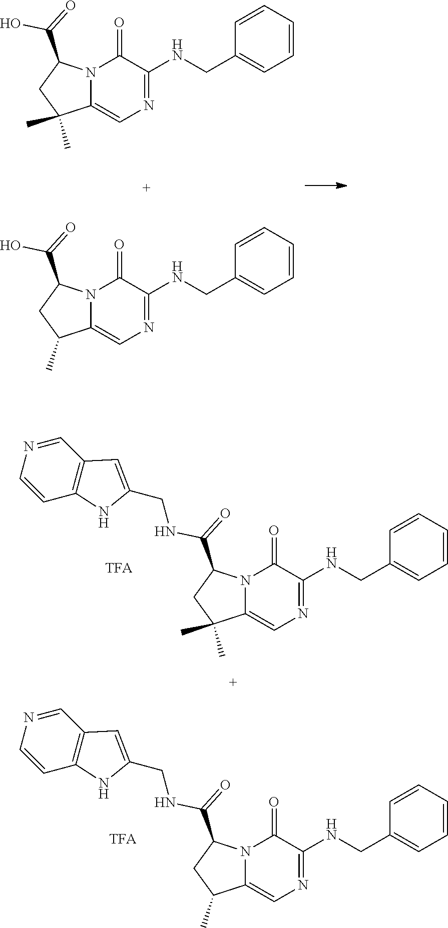

Step 9: A 50 mL round bottom flask was charged with a mixture of (6S,8R)-3-(benzylamino)-8-methyl-4-oxo-4,6,7,8-tetrahydropyrrolo[1,2-a]pyrazine-6-carboxylic acid, (S)-3-(benzylamino)-8,8-dimethyl-4-oxo-4,6,7,8-tetrahydropyrrolo[1,2-a]pyrazine-6-carboxylic acid, (1H-pyrrolo[3,2-c]pyridin-2-yl)methanamine (38 mg, 0.26 mmol), and DMF (2 mL). HATU (91 mg, 0.24 mmol) and DIEA (84 mg, 0.11 mL, 0.65 mmol) were then added to the reaction mixture. After completion of the reaction, the mixture was concentrated and the residue was purified by prep HPLC. Pure fractions were collected and concentrated to afford (S)—N-((1H-pyrrolo[3,2-c]pyridin-2-yl)methyl)-3-(benzylamino)-8,8-dimethyl-4-oxo-4,6,7,8-tetrahydropyrrolo[1,2-a]pyrazine-6-carboxamide, trifluoroacetate (4.4 mg) and (6S,8R)—N-((1H-pyrrolo[3,2-c]pyridin-2-yl)methyl)-3-(benzylamino)-8-methyl-4-oxo-4,6,7,8-tetrahydropyrrolo[1,2-a]pyrazine-6-carboxamide, trifluoroacetate (19.9 mg), to afford a combined 21% yield.

The following compounds were prepared according to the foregoing procedures using the appropriate amines, except that 7N NH₃ in MeOH was not added in the hydrogenolysis step:

| | Cmp |
|---|---|
| Compound Name | No. |
| (S)-N-((1H-pyrrolo[3,2-c]pyridin-2-yl)methyl)-3-(benzylamino)-1- | 229 |
| chloro-8,8-dimethyl-4-oxo-4,6,7,8-tetrahydropyrrolo[1,2- | |
| a]pyrazine-6-carboxamide trifluoroacetate | |
| (6S,8R)-N-((1H-pyrrolo[3,2-c]pyridin-2-yl)methyl)-3-(benzyl- | 230 |
| amino)-1-chloro-8-methyl-4-oxo-4,6,7,8-tetrahydropyrrolo[1,2- | |
| a]pyrazine-6-carboxamide trifluoroacetate | |

### Example 56

### Preparation of (S)—N-((1H-pyrrolo[3,2-c]pyridin-2-yl)methyl)-3′-(benzylamino)-1′-chloro-4′-oxo-6′,7′-dihydro-4′H-spiro[cyclopentane-1,8′-pyrrolo[1,2-a]pyrazine]-6′-carboxamide trifluoroacetate and (S)—N-((1H-pyrrolo[3,2-c]pyridin-2-yl)methyl)-3′-amino-1′-chloro-4′-oxo-6′,7′-dihydro-4′H-spiro[cyclopentane-1,8′-pyrrolo[1,2-a]pyrazine]-6′-carboxamide trifluoroacetate

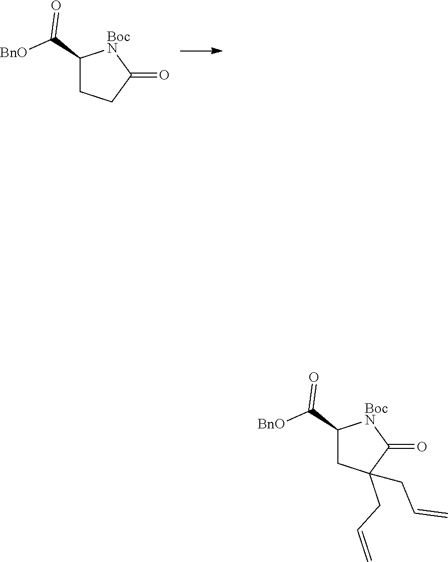 [SMILES: CC(C)(C)OC(=O)N1C(=O)CC[C@H]1C(=O)OCc1ccccc1 ; C=CCC1(CC=C)C[C@@H](C(=O)OCc2ccccc2)N(C(=O)OC(C)(C)C)C1=O]

Step 1: A solution of 2-benzyl 1-(tert-butyl) (S)-5-oxopyrrolidine-1,2-dicarboxylate (10 g, 31 mmol) in THF (65 mL) was cooled to −78° C. LiHMDS (1 M in THF, 34 mL, 34 mmol) was added over 5 min and the resulting solution was stirred for 30 min. Allyl bromide (4.2 g, 34 mmol) in THF (20 mL) was added dropwise to the solution. After 1 hour of stirring, LiHMDS (1 M in THF, 34 mL, 34 mmol) and allyl bromide (4.2 g, 34 mmol) in THF (20 mL) were added and the reaction mixture was stirred for 1 additional h. The reaction was quenched with aq. NH₄Cl and extracted with EtOAc 3×. The combined organics were washed with brine, dried with Na₂SO₄, and purified by column chromatography (heptane-EtOAc gradient) to afford the pure product 2-benzyl 1-(tert-butyl) (S)-4,4-diallyl-5-oxopyrrolidine-1,2-dicarboxylate (1.2 g, 9.6% yield) as a colorless oil.

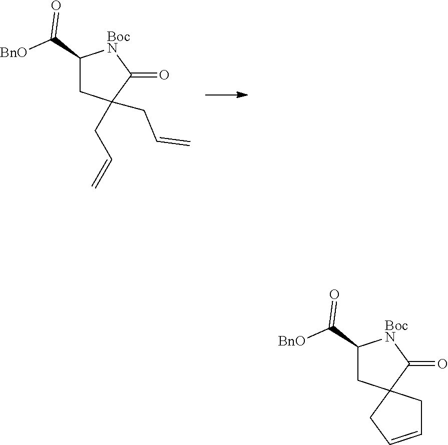 [SMILES: C=CCC1(CC=C)C[C@@H](C(=O)OCc2ccccc2)N(C(=O)OC(C)(C)C)C1=O ; CC(C)(C)OC(=O)N1C(=O)C2(CC=CC2)C[C@H]1C(=O)OCc1ccccc1]

Step 2: A solution of 2-benzyl 1-(tert-butyl) (S)-4,4-diallyl-5-oxopyrrolidine-1,2-dicarboxylate (1.2 g, 3.0 mmol) in DCM (60 mL) was purged with Ar. Grubbs catalyst II (0.13 g, 0.15 mmol) was added to the mixture and the reaction was stirred for 2 d. The reaction mixture was washed with H₂O and brine, dried over Na₂SO₄, and purified by chromatography (heptane-EtOAc) to afford 3-benzyl 2-(tert-butyl) (S)-1-oxo-2-azaspiro[4.4]non-7-ene-2,3-dicarboxylate (798 mg, 72% yield) as a colorless oil.

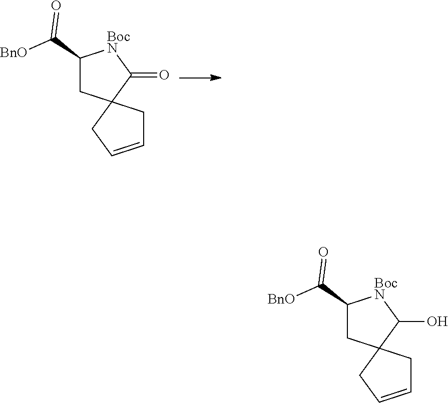 [SMILES: CC(C)(C)OC(=O)N1C(=O)C2(CC=CC2)C[C@H]1C(=O)OCc1ccccc1 ; CC(C)(C)OC(=O)N1C(O)C2(CC=CC2)C[C@H]1C(=O)OCc1ccccc1]

Step 3: The above compound was synthesized according to Example 2, step 2 to afford 3-benzyl 2-(tert-butyl) (3S)-1-hydroxy-2-azaspiro[4.4]non-7-ene-2,3-dicarboxylate.

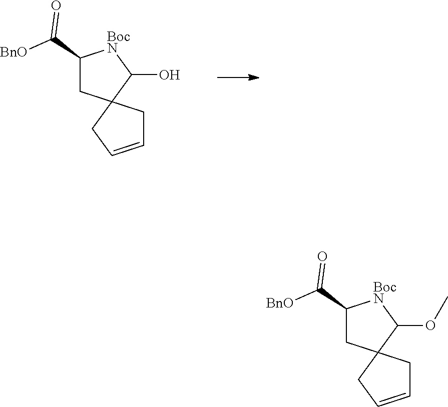 [SMILES: CC(C)(C)OC(=O)N1C(O)C2(CC=CC2)C[C@H]1C(=O)OCc1ccccc1 ; COC1N(C(=O)OC(C)(C)C)[C@H](C(=O)OCc2ccccc2)CC12CC=CC2]

Step 4: 3-Benzyl 2-(tert-butyl) (3S)-1-hydroxy-2-azaspiro[4.4]non-7-ene-2,3-dicarboxylate (832 mg, 2.23 mmol) and pTsOH (212 mg, 1.11 mmol) were dissolved in MeOH (8 mL) and stirred for 2 d. The reaction was concentrated and re-suspended in EtOAc. The organics were washed with sat. NaHCO₃, brine, and dried over Na₂SO₄ to afford 3-benzyl 2-(tert-butyl) (3S)-1-methoxy-2-azaspiro[4.4]non-7-ene-2,3-dicarboxylate as a yellow oil (598 mg, 69.3% yield).

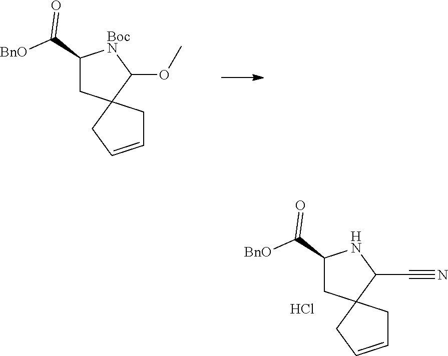 [SMILES: COC1N(C(=O)OC(C)(C)C)[C@H](C(=O)OCc2ccccc2)CC12CC=CC2 ; N#CC1N[C@H](C(=O)OCc2ccccc2)CC12CC=CC2 ; Cl]

Step 5: TMSCN (459 mg, 4.63 mmol) and BF₃—OEt₂ (657 mg, 4.63 mmol) were added to a solution of 3-benzyl 2-(tert-butyl) (3S)-1-methoxy-2-azaspiro[4.4]non-7-ene-2,3-dicarboxylate (598 mg, 1.54 mmol) in DCM (15 mL) cooled to −78° C. The mixture was stirred for 2 h, at which time it was quenched with sat. aq. K₂CO₃, warmed to room temperature, and extracted twice with DCM. The combined organics were washed with brine, dried with Na₂SO₄, and purified by column chromatography (heptane-EtOAc gradient). Pure fractions were collected and treated with 3 M HCl in CPME. After stirring for 1 h, the mixture was concentrated to afford benzyl (3S)-1-cyano-2-azaspiro[4.4]non-7-ene-3-carboxylate hydrochloride (500 mg, 102% yield) as a colorless oil.

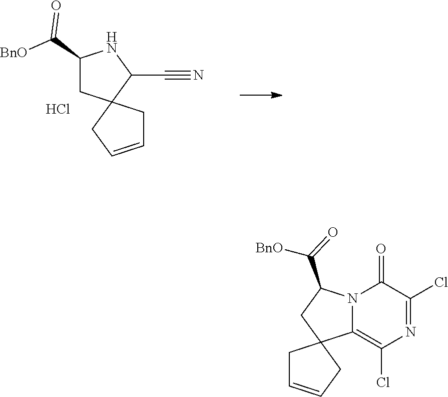 [SMILES: N#CC1N[C@H](C(=O)OCc2ccccc2)CC12CC=CC2 ; Cl ; O=C(OCc1ccccc1)[C@@H]1CC2(CC=CC2)c2c(Cl)nc(Cl)c(=O)n21]

Step 6: Benzyl (3S)-1-cyano-2-azaspiro[4.4]non-7-ene-3-carboxylate hydrochloride (500 mg, 1.57 mmol) in DME (5 mL) was added to a solution of oxalyl chloride (498 mg, 3.92 mmol) and DME (5 mL) at 0° C. The reaction was warmed to room temperature and then heated at 85° C. and stirred for 4 h. One drop of DMF was added to the reaction mixture with continued stirring for an additional 1 h. The crude material was purified by column chromatography (heptane-EtOAc gradient) to afford benzyl (S)-1′,3′-dichloro-4′-oxo-6′,7′-dihydro-4′H-spiro[cyclopentane-1,8′-pyrrolo[1,2-a]pyrazin]-3-ene-6′-carboxylate 210 mg, 34.2% yield) as a clear oil.

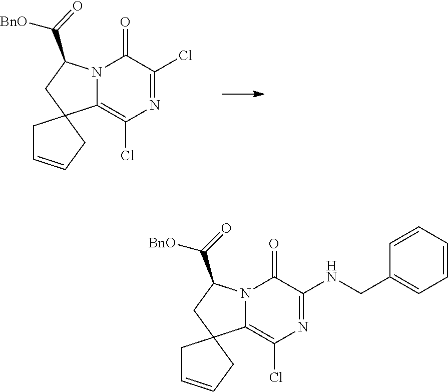 [SMILES: O=C(OCc1ccccc1)[C@@H]1CC2(CC=CC2)c2c(Cl)nc(Cl)c(=O)n21 ; O=C(OCc1ccccc1)[C@@H]1CC2(CC=CC2)c2c(Cl)nc(NCc3ccccc3)c(=O)n21]

Step 7: The above compound was synthesized according to General Chloro Displacement Procedure A to afford benzyl (S)-3′-(benzylamino)-1′-chloro-4′-oxo-6′,7′-dihydro-4′H-spiro[cyclopentane-1,8′-pyrrolo[1,2-a]pyrazin]-3-ene-6′-carboxylate (72 mg, 69% yield).

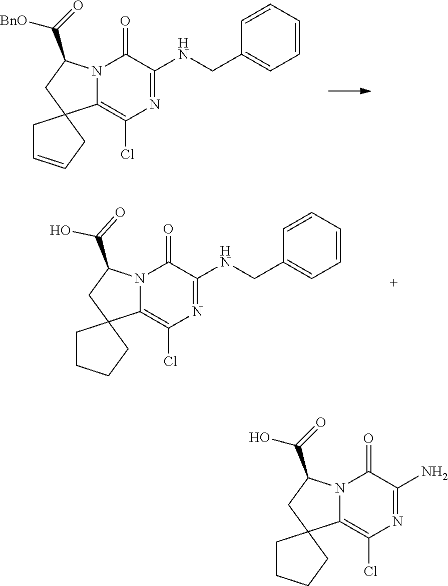 [SMILES: O=C(OCc1ccccc1)[C@@H]1CC2(CC=CC2)c2c(Cl)nc(NCc3ccccc3)c(=O)n21 ; O=C(O)[C@@H]1CC2(CCCC2)c2c(Cl)nc(NCc3ccccc3)c(=O)n21 ; Nc1nc(Cl)c2n(c1=O)[C@H](C(=O)O)CC21CCCC1]

Step 8: The above compounds were synthesized according to General Hydrogenolysis Procedure B, except that the reaction was performed in a 1:1 solution of EtOH:EtOAc to afford (S)-3′-amino-1′-chloro-4′-oxo-6′,7′-dihydro-4′H-spiro[cyclopentane-1,8′-pyrrolo[1,2-a]pyrazine]-6′-carboxylic acid and (S)-3′-(benzylamino)-1′-chloro-4′-oxo-6′,7′-dihydro-4′H-spiro[cyclopentane-1,8′-pyrrolo[1,2-a]pyrazine]-6′-carboxylic acid (quantitative yield).

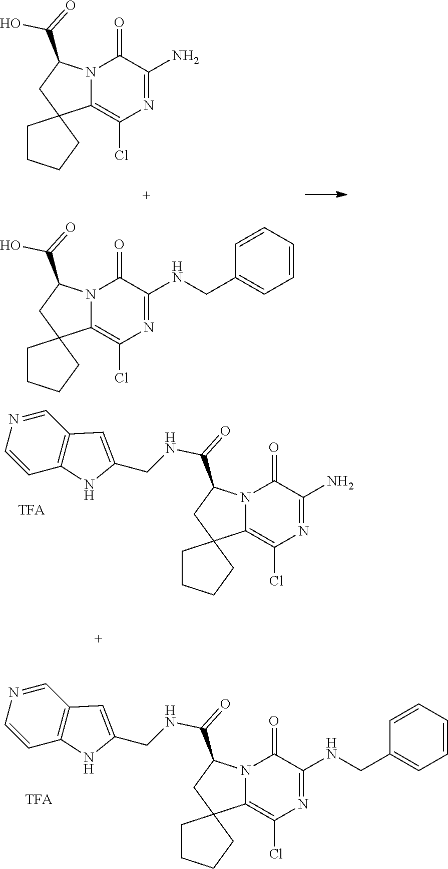

Step 9: The above compounds were synthesized according to General Amide Coupling Procedure A, except that prep HPLC was used for purification to afford (S)—N-((1H-pyrrolo[3,2-c]pyridin-2-yl)methyl)-3′-(benzylamino)-1′-chloro-4′-oxo-6′,7′-dihydro-4′H-spiro[cyclopentane-1,8′-pyrrolo[1,2-a]pyrazine]-6′-carboxamide trifluoroacetate (9.8 mg, 10% yield) and (S)—N-((1H-pyrrolo[3,2-c]pyridin-2-yl)methyl)-3′-amino-1′-chloro-4′-oxo-6′,7′-dihydro-4′H-spiro[cyclopentane-1,8′-pyrrolo[1,2-a]pyrazine]-6′-carboxamide trifluoroacetate (7.8 mg, 8% yield).

### Example 57

### Preparation of (S)—N-((1H-pyrrolo[3,2-c]pyridin-2-yl)methyl)-3′-(benzylamino)-1′-chloro-4′-oxo-6′,7′-dihydro-4′H-spiro[cyclopentane-1,8′-pyrrolo[1,2-a]pyrazin]-3-ene-6′-carboxamide trifluoroacetate

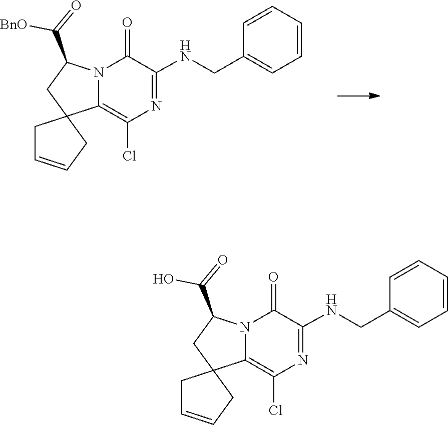 [SMILES: O=C(OCc1ccccc1)[C@@H]1CC2(CC=CC2)c2c(Cl)nc(NCc3ccccc3)c(=O)n21 ; O=C(O)[C@@H]1CC2(CC=CC2)c2c(Cl)nc(NCc3ccccc3)c(=O)n21]

Step 1: Benzyl (S)-3′-(benzylamino)-4′-oxo-6′,7′-dihydro-4′H-spiro[cyclopentane-1,8′-pyrrolo[1,2-a]pyrazin]-3-ene-6′-carboxylate (15 mg, 35 μmol) and 1 M LiOH (70 μL) were dissolved in THE (0.5 mL) and MeOH (0.5 mL), and the mixture was stirred overnight at room temperature. 1 N HCl was added, and the mixture was extracted 3× with EtOAc. The combined organics were concentrated to afford (S)-3′-(benzylamino)-4′-oxo-6′,7′-dihydro-4′H-spiro[cyclopentane-1,8′-pyrrolo[1,2-a]pyrazin]-3-ene-6′-carboxylic acid (12 mg, 100% yield).

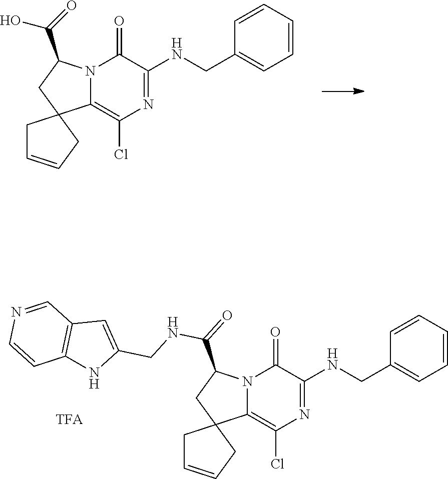 [SMILES: O=C(O)[C@@H]1CC2(CC=CC2)c2c(Cl)nc(NCc3ccccc3)c(=O)n21 ; O=C(NCc1cc2cnccc2[nH]1)[C@@H]1CC2(CC=CC2)c2c(Cl)nc(NCc3ccccc3)c(=O)n21]

Step 2: The above compounds were synthesized according to General Amide Coupling Procedure A, except that prep HPLC was used after column chromatography for purification to afford (S)—N-((1H-pyrrolo[3,2-c]pyridin-2-yl)methyl)-3′-(benzylamino)-1′-chloro-4′-oxo-6′,7′-dihydro-4′H-spiro[cyclopentane-1,8′-pyrrolo[1,2-a]pyrazin]-3-ene-6′-carboxamide trifluoroacetate (1.7 mg, 8% yield).

### Example 58

### Preparation of (S)—N—((S)-1-(5,6-dihydro-4H-thieno[2,3-c]pyrrol-2-yl)ethyl)-4-oxo-3-(((R)-1-phenylpropyl)amino)-4,6,7,8-tetrahydropyrrolo[1,2-a]pyrazine-6-carboxamide trifluoroacetate and (S)—N—((R)-1-(5,6-dihydro-4H-thieno[2,3-c]pyrrol-2-yl)ethyl)-4-oxo-3-(((R)-1-phenylpropyl)amino)-4,6,7,8-tetrahydropyrrolo[1,2-a]pyrazine-6-carboxamide trifluoroacetate

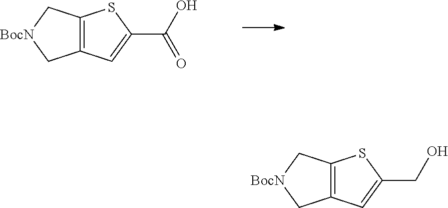 [SMILES: CC(C)(C)OC(=O)N1Cc2cc(C(=O)O)sc2C1 ; CC(C)(C)OC(=O)N1Cc2cc(CO)sc2C1]

Step 1: A solution of 5-(tert-Butoxycarbonyl)-5,6-dihydro-4H-thieno[2,3-c]pyrrole-2-carboxylic acid (3.00 g, 11.1 mmol), N-methylmorpholine (2.8 mL, 25.6 mmol), and THF (30 mL) was cooled to 0° C. Isobutyl chloroformate (1.6 mL, 12.3 mmol) was added and the reaction was warmed to room temperature and stirred for 1 h. The precipitate was filtered off and the remaining solute was cooled to 0° C. NaBH₄ (1.77 g, 46.8 mmol) was dissolved in 2 mL of water and added to the reaction slowly. The reaction was stirred for 30 min at 0° C. then warmed to room temperature. The resulting reaction was allowed to stir for an additional 16 h. The reaction was quenched with 5 mL of water and extracted with EtOAc three times. The organic layers were combined, washed with brine, dried with Na₂SO₄, vacuum filtered, and conc in vacuo. The crude material was chromatographed with heptane-EtOAc gradient to afford tert-butyl 2-(hydroxymethyl)-4,6-dihydro-5H-thieno[2,3-c]pyrrole-5-carboxylate as a white solid (1.9 g, 67% yield).

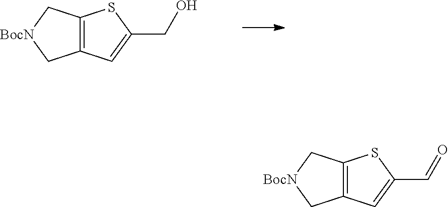 [SMILES: CC(C)(C)OC(=O)N1Cc2cc(CO)sc2C1 ; CC(C)(C)OC(=O)N1Cc2cc(C=O)sc2C1]

Step 2: To a solution of tert-butyl 2-(hydroxymethyl)-4,6-dihydro-5H-thieno[2,3-c]pyrrole-5-carboxylate (1.9 g, 7.4 mmol) in DCM (40 mL) was added manganese dioxide (6.5 g, 74 mmol). The mixture was allowed to stir at room temperature for 16 h under N₂ atmosphere then filtered through a plug of diatomaceous earth. The crude material was conc in vacuo then chromatographed with heptane-EtOAc gradient to afford tert-butyl 2-formyl-4,6-dihydro-5H-thieno[2,3-c]pyrrole-5-carboxylate (786 mg, 42% yield).

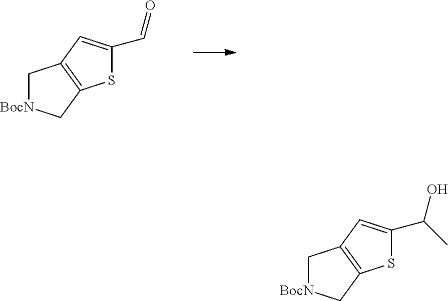 [SMILES: CC(C)(C)OC(=O)N1Cc2cc(C=O)sc2C1 ; CC(O)c1cc2c(s1)CN(C(=O)OC(C)(C)C)C2]

Step 3: tert-Butyl 2-formyl-4,6-dihydro-5H-thieno[2,3-c]pyrrole-5-carboxylate (780 mg, 3.08 mmol) was added to dry THF (20 mL) under a blanket of Ar. The solution was cooled to −78° C. and methyl magnesium bromide (2.57 mL, 3 M in Et₂O, 7.70 mmol) was added dropwise. The solution was stirred at −78° C. for 20 min and then warmed to 0° C. over 2 h. 1 N HCl was added, and the solution was extracted with EtOAc. The organic layers were combined, dried over Na₂SO₄, filtered, and concentrated to afford the pure product tert-butyl 2-(1-hydroxyethyl)-4,6-dihydro-5H-thieno[2,3-c]pyrrole-5-carboxylate (734 mg, 89% yield).

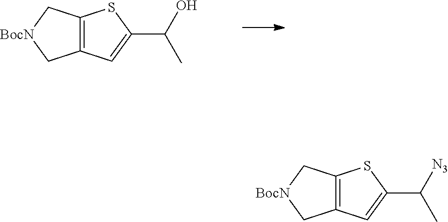 [SMILES: CC(O)c1cc2c(s1)CN(C(=O)OC(C)(C)C)C2 ; CC(N=[N+]=[N-])c1cc2c(s1)CN(C(=O)OC(C)(C)C)C2]

Step 4: tert-Butyl 2-(1-hydroxyethyl)-4,6-dihydro-5H-thieno[2,3-c]pyrrole-5-carboxylate (679 mg, 2.52 mmol) was added to THF (10 mL) under N₂. Diphenyl phosphorylazide (813 μL, 3.78 mmol) was added to the reaction mixture. After cooling to 0° C., 1,8-diazabicyclo[5.4.0]undec-7-ene (570 μL, 3.78 mmol) was added, the reaction flask was sealed, and warmed to 20° C. for 5 min. The resulting clear orange solution was heated at 65° C. for 20 h. The reaction was quenched with water and extracted with EtOAc three times. The organic layers were combined, washed with brine, dried over anhyd Na₂SO₄, vacuum filtered, and conc in vacuo. The crude material was chromatographed with heptane-EtOAc gradient to afford tert-butyl 2-(1-azidoethyl)-4,6-dihydro-5H-thieno[2,3-c]pyrrole-5-carboxylate (531 mg, 72% yield).

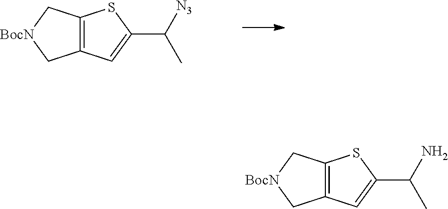 [SMILES: CC(N=[N+]=[N-])c1cc2c(s1)CN(C(=O)OC(C)(C)C)C2 ; CC(N)c1cc2c(s1)CN(C(=O)OC(C)(C)C)C2]

Step 5: To a solution of tert-butyl 2-(1-azidoethyl)-4,6-dihydro-5H-thieno[2,3-c]pyrrole-5-carboxylate (531 mg, 1.80 mmol) in THF (5 mL) was added triphenylphosphine (710 mg, 2.71 mmol) and water (715 μL, 39.7 mmol). After stirring for 20 h, the reaction was quenched with 1 N KHSO₄ solution and washed with ether 3 times. The ethereal layers were discarded. The aqueous layer was treated with 5 N NaOH solution to pH ˜11 and was extracted with EtOAc 3 times. The organic layers were combined, washed with brine, dried over anhydrous Na₂SO₄, filtered, and conc in vacuo. The crude material was chromatographed with heptane-EtOAc gradient to afford the product tert-butyl 2-(1-aminoethyl)-4,6-dihydro-5H-thieno[2,3-c]pyrrole-5-carboxylate (303 mg, 63% yield).

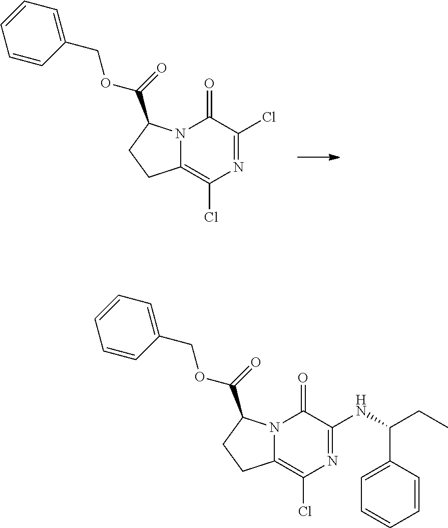 [SMILES: O=C(OCc1ccccc1)[C@@H]1CCc2c(Cl)nc(Cl)c(=O)n21 ; CC[C@@H](Nc1nc(Cl)c2n(c1=O)[C@H](C(=O)OCc1ccccc1)CC2)c1ccccc1]

Step 6: A mixture of benzyl (S)-1,3-dichloro-4-oxo-4,6,7,8-tetrahydropyrrolo[1,2-a]pyrazine-6-carboxylate (0.152 g, 0.448 mmol), (R)-1-phenylpropan-1-amine (66.7 mg, 0.493 mmol), and DIEA (57.9 mg, 0.448 mmol) in acetonitrile (2.2 mL) was heated at 85° C. for 16 h. The crude material was concentrated, chromatographed with heptane-EtOAc gradient to afford benzyl (S)-1-chloro-4-oxo-3-(((R)-1-phenylpropyl)amino)-4,6,7,8-tetrahydropyrrolo[1,2-a]pyrazine-6-carboxylate as an off-white solid (71 mg, 36% yield).

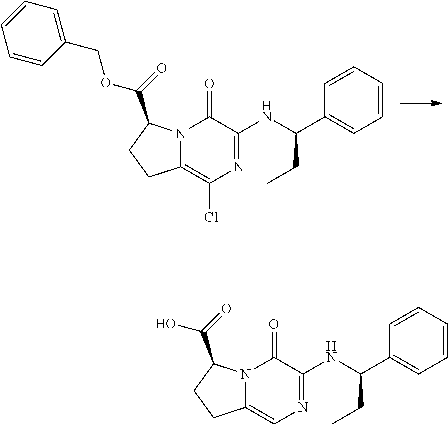 [SMILES: CC[C@@H](Nc1nc(Cl)c2n(c1=O)[C@H](C(=O)OCc1ccccc1)CC2)c1ccccc1 ; CC[C@@H](Nc1ncc2n(c1=O)[C@H](C(=O)O)CC2)c1ccccc1]

Step 7: A solution of benzyl (S)-1-chloro-4-oxo-3-(((R)-1-phenylpropyl)amino)-4,6,7,8-tetrahydropyrrolo[1,2-a]pyrazine-6-carboxylate (71.4 mg, 0.163 mmol) in MeOH (3.2 mL) was put under N₂ and treated with DIEA (42 mg, 0.33 mmol) and Pd/C (9.9 mg, 10% wt.). Air was removed by evacuating and back filling with H₂ 5 times. The reaction was stirred under H₂ at room temp for 16 h. The mixture was filtered (0.2 μm syringe filter) then volatiles were removed in vacuo to afford (S)-4-oxo-3-(((R)-1-phenylpropyl)amino)-4,6,7,8-tetrahydropyrrolo[1,2-a]pyrazine-6-carboxylic acid. This material was used in the next synthetic step without purification.

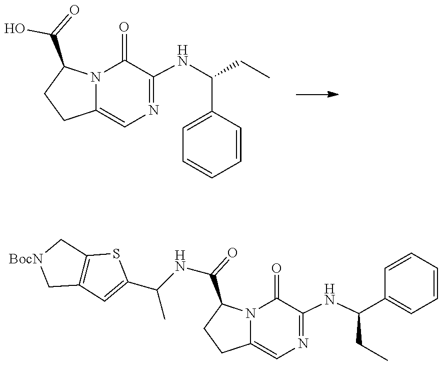 [SMILES: CC[C@@H](Nc1ncc2n(c1=O)[C@H](C(=O)O)CC2)c1ccccc1 ; CC[C@@H](Nc1ncc2n(c1=O)[C@H](C(=O)NC(C)c1cc3c(s1)CN(C(=O)OC(C)(C)C)C3)CC2)c1ccccc1]

Step 8: A solution of (S)-4-oxo-3-(((R)-1-phenylpropyl)amino)-4,6,7,8-tetrahydropyrrolo[1,2-a]pyrazine-6-carboxylic acid (51.1 mg, 0.163 mmol) in DMF (1.2 mL) was treated with DIEA (42 mg, 0.33 mmol) and tert-butyl 2-(1-aminoethyl)-4,6-dihydro-5H-thieno[2,3-c]pyrrole-5-carboxylate (53 mg, 0.20 mmol) at room temp. HATU (68.2 mg, 0.179 mmol) was added at 0° C. then the reaction was warmed to 20° C. and stirred for 16 h. The crude mixture was conc in vacuo, then chromatographed with a heptane-EtOAc gradient to afford (S)—N-((6-amino-2-methylpyridin-3-yl)methyl)-3-((3,5-dimethylbenzyl)amino)-4-oxo-4,6,7,8-tetrahydropyrrolo[1,2-a]pyrazine-6-carboxamide as a white solid (92 mg, 66% yield).

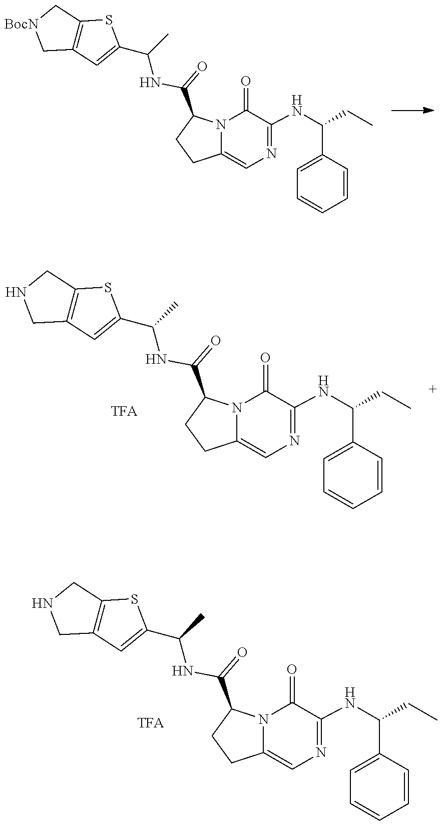 [SMILES: CC[C@@H](Nc1ncc2n(c1=O)[C@H](C(=O)NC(C)c1cc3c(s1)CN(C(=O)OC(C)(C)C)C3)CC2)c1ccccc1 ; CC[C@@H](Nc1ncc2n(c1=O)[C@H](C(=O)N[C@@H](C)c1cc3c(s1)CNC3)CC2)c1ccccc1 ; CC[C@@H](Nc1ncc2n(c1=O)[C@H](C(=O)N[C@H](C)c1cc3c(s1)CNC3)CC2)c1ccccc1]

Step 9: A solution of tert-butyl 2-(1-((S)-4-oxo-3-(((R)-1-phenylpropyl)amino)-4,6,7,8-tetrahydropyrrolo[1,2-a]pyrazine-6-carboxamido)ethyl)-4,6-dihydro-5H-thieno[2,3-c]pyrrole-5-carboxylate (91.9 mg, 0.163 mmol) in MeOH (1 mL) was put under N₂. HCl in MeOH (2 mL, 3 M solution) was added at 0° C. The solution was warmed to 20° C. and stirred for 16 h. The crude material was conc in vacuo then purified by prep HPLC to afford (S)—N—((S)-1-(5,6-dihydro-4H-thieno[2,3-c]pyrrol-2-yl)ethyl)-4-oxo-3-(((R)-1-phenylpropyl)amino)-4,6,7,8-tetrahydropyrrolo[1,2-a]pyrazine-6-carboxamide trifluoroacetate (14.4 mg, 13% yield) and (S)—N—((R)-1-(5,6-dihydro-4H-thieno[2,3-c]pyrrol-2-yl)ethyl)-4-oxo-3-(((R)-1-phenylpropyl)amino)-4,6,7,8-tetrahydropyrrolo[1,2-a]pyrazine-6-carboxamide trifluoroacetate (14.2 mg, 13% yield), respectively.

The following compounds were prepared according to the foregoing procedures using the appropriate amines:

| | Cmp |
|---|---|
| Compound Name | No. |
| (S)-N-((R)-1-(5,6-dihydro-4H-thieno[2,3-c]pyrrol-2-yl)ethyl)-3- | 269 |
| (((R)-2,2-dimethyl-1-phenylpropyl)amino)-4-oxo-4,6,7,8-tetra- | |
| hydropyrrolo[1,2-a]pyrazine-6-carboxamide trifluoroacetate | |
| (S)-N-((S)-1-(5,6-dihydro-4H-thieno[2,3-c]pyrrol-2-yl)ethyl)-3- | 270 |
| (((R)-2,2-dimethyl-1-phenylpropyl)amino)-4-oxo-4,6,7,8-tetra- | |
| hydropyrrolo[1,2-a]pyrazine-6-carboxamide trifluoroacetate | |
| (S)-3-(((R)-cyclopropyl(phenyl)methyl)amino)-N-((R)-1-(5,6- | 272 |
| dihydro-4H-thieno[2,3-c]pyrrol-2-yl)ethyl)-4-oxo-4,6,7,8-tetra- | |
| hydropyrrolo[1,2-a]pyrazine-6-carboxamide trifluoroacetate | |
| (S)-3-(((R)-cyclopropyl(phenyl)methyl)amino)-N-((S)-1-(5,6-di- | 273 |
| hydro-4H-thieno[2,3-c]pyrrol-2-yl)ethyl)-4-oxo-4,6,7,8-tetrahydro- | |
| pyrrolo[1,2-a]pyrazine-6-carboxamide trifluoroacetate | |
| (S)-N-((R)-1-(5,6-dihydro-4H-thieno[2,3-c]pyrrol-2-yl)ethyl)-3- | 274 |
| (((R)-2-methyl-1-phenylpropyl)amino)-4-oxo-4,6,7,8-tetrahydro- | |
| pyrrolo[1,2-a]pyrazine-6-carboxamide trifluoroacetate | |
| (S)-N-((S)-1-(5,6-dihydro-4H-thieno[2,3-c]pyrrol-2-yl)ethyl)-3- | 275 |
| (((R)-2-methyl-1-phenylpropyl)amino)-4-oxo-4,6,7,8-tetrahydro- | |
| pyrrolo[1,2-a]pyrazine-6-carboxamide trifluoroacetate | |

### Example 59

### Preparation of (S)-3-(((1-benzylcyclohexyl)methyl)amino)-N-((5,6-dihydro-4H-thieno[2,3-c]pyrrol-2-yl)methyl)-4-oxo-4,6,7,8-tetrahydropyrrolo[1,2-a]pyrazine-6-carboxamide trifluoroacetate

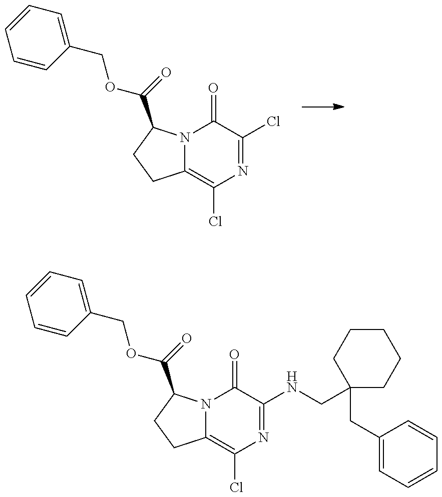 [SMILES: O=C(OCc1ccccc1)[C@@H]1CCc2c(Cl)nc(Cl)c(=O)n21 ; O=C(OCc1ccccc1)[C@@H]1CCc2c(Cl)nc(NCC3(Cc4ccccc4)CCCCC3)c(=O)n21]

Step 1: General Chloro Displacement Procedure A was used to synthesize benzyl (S)-3-(((1-benzylcyclohexyl)methyl)amino)-1-chloro-4-oxo-4,6,7,8-tetrahydropyrrolo[1,2-a]pyrazine-6-carboxylate as an oil (119 mg, 55% yield).

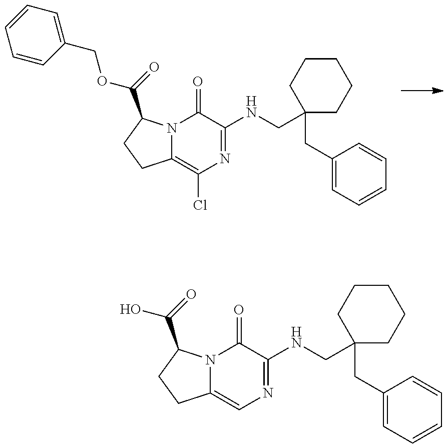 [SMILES: O=C(OCc1ccccc1)[C@@H]1CCc2c(Cl)nc(NCC3(Cc4ccccc4)CCCCC3)c(=O)n21 ; O=C(O)[C@@H]1CCc2cnc(NCC3(Cc4ccccc4)CCCCC3)c(=O)n21]

Step 2: General Hydrogenolysis Procedure B was used to synthesize (S)-3-(((1-benzylcyclohexyl)methyl)amino)-4-oxo-4,6,7,8-tetrahydropyrrolo[1,2-a]pyrazine-6-carboxylic acid as a clear oil (quantitative yield).

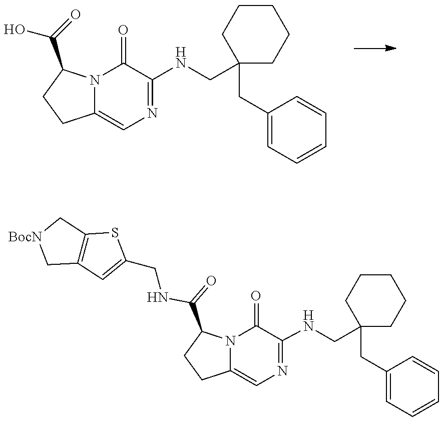 [SMILES: O=C(O)[C@@H]1CCc2cnc(NCC3(Cc4ccccc4)CCCCC3)c(=O)n21 ; CC(C)(C)OC(=O)N1Cc2cc(CNC(=O)[C@@H]3CCc4cnc(NCC5(Cc6ccccc6)CCCCC5)c(=O)n43)sc2C1]

Step 3: General Amide Coupling Procedure A was used, except that the crude material was purified by chromatography with DCM-MeOH gradient, to afford tert-butyl (S)-2-((3-(((1-benzylcyclohexyl)methyl)amino)-4-oxo-4,6,7,8-tetrahydropyrrolo[1,2-a]pyrazine-6-carboxamido)methyl)-4,6-dihydro-5H-thieno[2,3-c]pyrrole-5-carboxylate (40 mg, 28% yield).

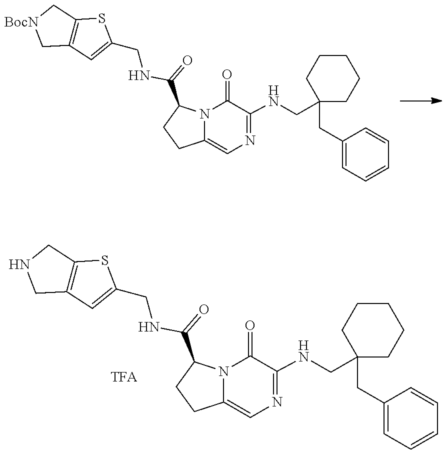 [SMILES: CC(C)(C)OC(=O)N1Cc2cc(CNC(=O)[C@@H]3CCc4cnc(NCC5(Cc6ccccc6)CCCCC5)c(=O)n43)sc2C1 ; O=C(NCc1cc2c(s1)CNC2)[C@@H]1CCc2cnc(NCC3(Cc4ccccc4)CCCCC3)c(=O)n21]

Step 4: General Boc Removal Procedure A was used, except that the crude material was purified by prep HPLC, to afford (S)-3-(((1-benzylcyclohexyl)methyl)amino)-N-((5,6-dihydro-4H-thieno[2,3-c]pyrrol-2-yl)methyl)-4-oxo-4,6,7,8-tetrahydropyrrolo[1,2-a]pyrazine-6-carboxamide trifluoroacetate (37 mg, 76% yield).

### Example 60

### Preparation of (S)—N-((5,6-dihydro-4H-thieno[2,3-c]pyrrol-2-yl)methyl)-4-oxo-3-(((S)-6-phenyl-2,3-dihydro-1H-inden-1-yl)amino)-4,6,7,8-tetrahydropyrrolo[1,2-a]pyrazine-6-carboxamide trifluoroacetate

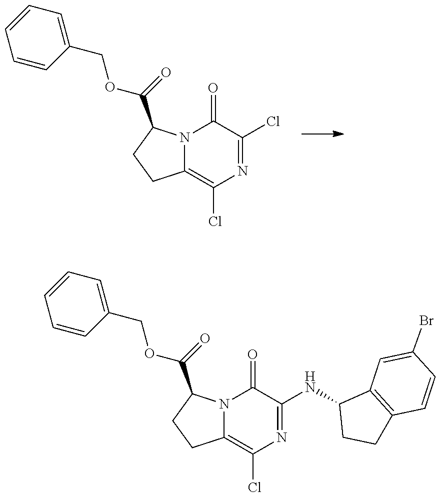 [SMILES: O=C(OCc1ccccc1)[C@@H]1CCc2c(Cl)nc(Cl)c(=O)n21 ; O=C(OCc1ccccc1)[C@@H]1CCc2c(Cl)nc(N[C@H]3CCc4ccc(Br)cc43)c(=O)n21]

Step 1: General Chloro Displacement Procedure A was used to afford benzyl (S)-3-(((S)-6-bromo-2,3-dihydro-1H-inden-1-yl)amino)-1-chloro-4-oxo-4,6,7,8-tetrahydropyrrolo[1,2-a]pyrazine-6-carboxylate (215 mg, 46% yield).

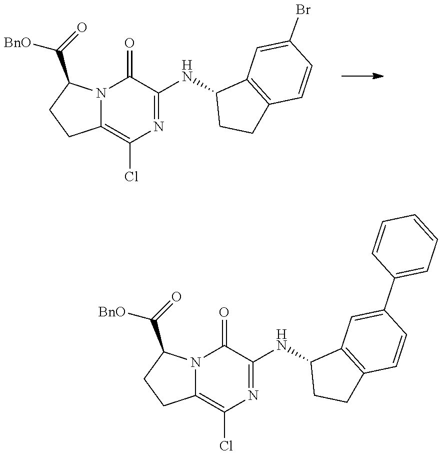 [SMILES: O=C(OCc1ccccc1)[C@@H]1CCc2c(Cl)nc(N[C@H]3CCc4ccc(Br)cc43)c(=O)n21 ; O=C(OCc1ccccc1)[C@@H]1CCc2c(Cl)nc(N[C@H]3CCc4ccc(-c5ccccc5)cc43)c(=O)n21]

Step 2: A microwave vial was charged with benzyl (S)-3-(((S)-6-bromo-2,3-dihydro-1H-inden-1-yl)amino)-1-chloro-4-oxo-4,6,7,8-tetrahydropyrrolo[1,2-a]pyrazine-6-carboxylate (214.7 mg, 0.417 mmol), phenylboronic acid (80 mg, 0.660 mmol), Pd(dppf)₂Cl₂ (33 mg, 0.045 mmol), and Cs₂CO₃ (407 mg, 1.25 mmol). Dioxane (4 mL) and water (0.4 mL) were added. The mixture was heated at 100° C. under microwave irradiation until completion. The reaction mixture was quenched with water, diluted with saturated aq. NH₄Cl solution, and extracted with DCM 3 times. The organic layers were combined, dried over Na₂SO₄, filtered, and conc in vacuo. The crude material was chromatographed with a heptane-EtOAc gradient to afford benzyl (S)-1-chloro-4-oxo-3-(((S)-6-phenyl-2,3-dihydro-1H-inden-1-yl)amino)-4,6,7,8-tetrahydropyrrolo[1,2-a]pyrazine-6-carboxylate as an off-white foam (137.3 mg, 64% yield).

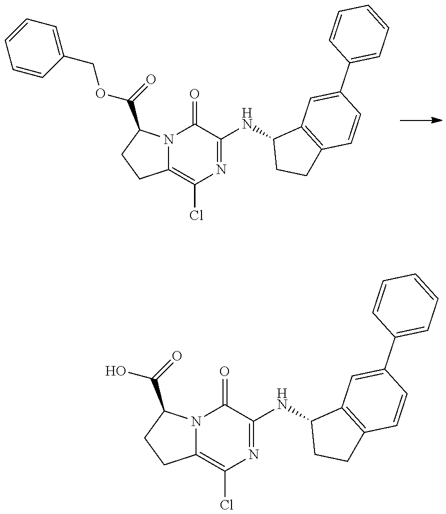 [SMILES: O=C(OCc1ccccc1)[C@@H]1CCc2c(Cl)nc(N[C@H]3CCc4ccc(-c5ccccc5)cc43)c(=O)n21 ; O=C(O)[C@@H]1CCc2c(Cl)nc(N[C@H]3CCc4ccc(-c5ccccc5)cc43)c(=O)n21]

Step 3: General Hydrogenolysis Procedure A was used to afford (S)-4-oxo-3-(((S)-6-phenyl-2,3-dihydro-1H-inden-1-yl)amino)-4,6,7,8-tetrahydropyrrolo[1,2-a]pyrazine-6-carboxylic acid (quantitative yield).

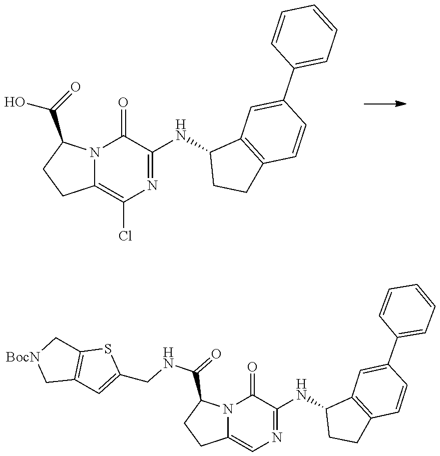 [SMILES: O=C(O)[C@@H]1CCc2c(Cl)nc(N[C@H]3CCc4ccc(-c5ccccc5)cc43)c(=O)n21 ; CC(C)(C)OC(=O)N1Cc2cc(CNC(=O)[C@@H]3CCc4cnc(N[C@H]5CCc6ccc(-c7ccccc7)cc65)c(=O)n43)sc2C1]

Step 4: General Amide Coupling Procedure A was used, except that the crude material was purified by chromatography with heptane-EtOAc gradient, to afford tert-butyl 2-(((S)-4-oxo-3-(((S)-6-phenyl-2,3-dihydro-1H-inden-1-yl)amino)-4,6,7,8-tetrahydropyrrolo[1,2-a]pyrazine-6-carboxamido)methyl)-4,6-dihydro-5H-thieno[2,3-c]pyrrole-5-carboxylate (79 mg, 47% yield).

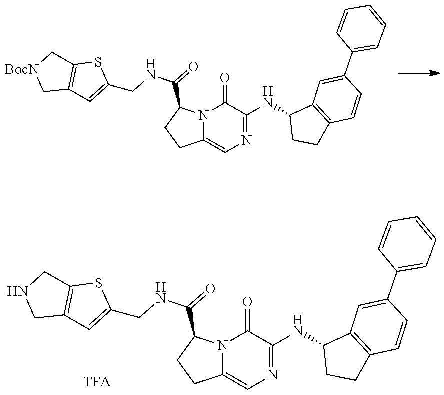 [SMILES: CC(C)(C)OC(=O)N1Cc2cc(CNC(=O)[C@@H]3CCc4cnc(N[C@H]5CCc6ccc(-c7ccccc7)cc65)c(=O)n43)sc2C1 ; O=C(NCc1cc2c(s1)CNC2)[C@@H]1CCc2cnc(N[C@H]3CCc4ccc(-c5ccccc5)cc43)c(=O)n21]

Step 5: General Boc Removal Procedure A was used, except that the crude material was purified by prep HPLC, to afford (S)—N-((5,6-dihydro-4H-thieno[2,3-c]pyrrol-2-yl)methyl)-4-oxo-3-(((S)-6-phenyl-2,3-dihydro-1H-inden-1-yl)amino)-4,6,7,8-tetrahydropyrrolo[1,2-a]pyrazine-6-carboxamide trifluoroacetate as an off-white solid (60 mg, 63% yield).

The following compound was prepared according to the foregoing procedure using the appropriate amine:

| | Cmp |
|---|---|
| Compound Name | No. |
| (S)-N-((5,6-dihydro-4H-thieno[2,3-c]pyrrol-2-yl)methyl)-4-oxo-3- | 260 |
| (((R)-6-phenyl-2,3-dihydro-1H-inden-1-yl)amino)-4,6,7,8-tetra- | |
| hydropyrrolo[1,2-a]pyrazine-6-carboxamide trifluoroacetate | |

### Example 61

### Preparation of (S)—N-((5,6-dihydro-4H-thieno[2,3-c]pyrrol-2-yl)methyl)-3-(((5-ethyl-[1,1′-biphenyl]-3-yl)methyl)amino)-4-oxo-4,6,7,8-tetrahydropyrrolo[1,2-a]pyrazine-6-carboxamide trifluoroacetate

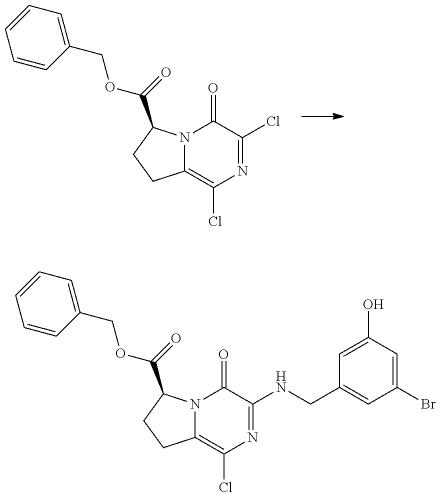 [SMILES: O=C(OCc1ccccc1)[C@@H]1CCc2c(Cl)nc(Cl)c(=O)n21 ; O=C(OCc1ccccc1)[C@@H]1CCc2c(Cl)nc(NCc3cc(O)cc(Br)c3)c(=O)n21]

Step 1: General Chloro Displacement Procedure A was used to afford benzyl (S)-3-((3-bromo-5-hydroxybenzyl)amino)-1-chloro-4-oxo-4,6,7,8-tetrahydropyrrolo[1,2-a]pyrazine-6-carboxylate as an off-white solid (237 mg, 63% yield).

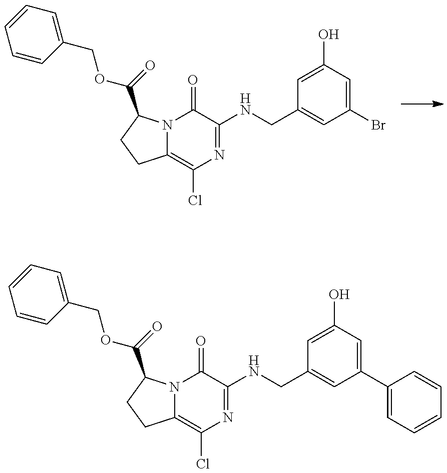 [SMILES: O=C(OCc1ccccc1)[C@@H]1CCc2c(Cl)nc(NCc3cc(O)cc(Br)c3)c(=O)n21 ; O=C(OCc1ccccc1)[C@@H]1CCc2c(Cl)nc(NCc3cc(O)cc(-c4ccccc4)c3)c(=O)n21]

Step 2: A microwave vial was charged with benzyl (S)-3-((3-bromo-5-hydroxybenzyl)amino)-1-chloro-4-oxo-4,6,7,8-tetrahydropyrrolo[1,2-a]pyrazine-6-carboxylate (107.9 mg, 0.214 mmol), phenylboronic acid (41.1 mg, 0.337 mmol), Pd(dppf)Cl₂ (156 mg, 10% wt.), Cs₂CO₃ (212 mg, 0.651 mmol), dioxane (2 mL), and water (0.2 mL). The mixture was heated at 100° C. for 45 min under microwave irradiation. The reaction mixture was quenched with water, diluted with saturated aq. NH₄Cl solution, and extracted with DCM 3 times. The organic layers were combined, washed with brine, dried over Na₂SO₄, filtered, and conc in vacuo. The crude material was chromatographed with heptane-EtOAc gradient to afford benzyl (S)-1-chloro-3-(((5-hydroxy-[1,1′-biphenyl]-3-yl)methyl)amino)-4-oxo-4,6,7,8-tetrahydropyrrolo[1,2-a]pyrazine-6-carboxylate as an off-white solid (49 mg, 45% yield).

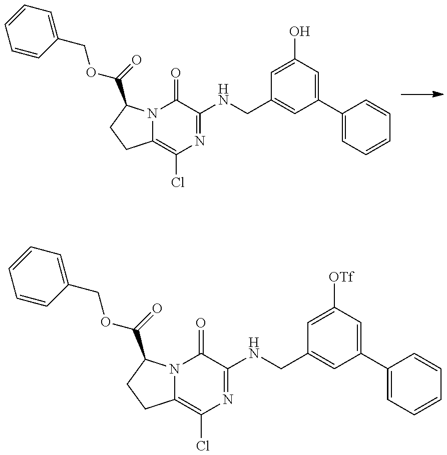 [SMILES: O=C(OCc1ccccc1)[C@@H]1CCc2c(Cl)nc(NCc3cc(O)cc(-c4ccccc4)c3)c(=O)n21 ; O=C(OCc1ccccc1)[C@@H]1CCc2c(Cl)nc(NCc3cc(OS(=O)(=O)C(F)(F)F)cc(-c4ccccc4)c3)c(=O)n21]

Step 3: A solution of benzyl (S)-1-chloro-3-(((5-hydroxy-[1,1′-biphenyl]-3-yl)methyl)amino)-4-oxo-4,6,7,8-tetrahydropyrrolo[1,2-a]pyrazine-6-carboxylate (48.6 mg, 0.097 mmol) in DCM (2 mL) was put under N₂ and treated with N-(5-chloropyridin-2-yl)-1,1,1-trifluoro-N-((trifluoromethyl)sulfonyl)methanesulfonamide (57.2 mg, 0.146 mmol), DIEA (34 μL, 0.2 mmol), and DMAP (5 mg, 0.04 mmol). The reaction was stirred at 20° C. for 16 h. The reaction mixture was then diluted with EtOAc, washed with 0.5 M HCl solution 2 times, washed with 0.5 M NaOH solution 2 times, washed with brine, dried over Na₂SO₄, filtered, and cone in vacuo. The crude material was chromatographed with heptane-EtOAc gradient to afford benzyl (S)-1-chloro-4-oxo-3-(((5-(((trifluoromethyl)sulfonyl)oxy)-[1,1′-biphenyl]-3-yl)methyl)amino)-4,6,7,8-tetrahydropyrrolo[1,2-a]pyrazine-6-carboxylate as a white partially solid material (48 mg, 78% yield).

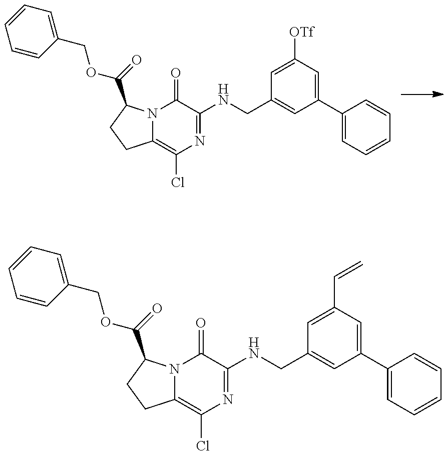 [SMILES: O=C(OCc1ccccc1)[C@@H]1CCc2c(Cl)nc(NCc3cc(OS(=O)(=O)C(F)(F)F)cc(-c4ccccc4)c3)c(=O)n21 ; C=Cc1cc(CNc2nc(Cl)c3n(c2=O)[C@H](C(=O)OCc2ccccc2)CC3)cc(-c2ccccc2)c1]

Step 4: A microwave vial was charged with benzyl (S)-1-chloro-4-oxo-3-(((5-(((trifluoromethyl)sulfonyl)oxy)-[1,1′-biphenyl]-3-yl)methyl)amino)-4,6,7,8-tetrahydropyrrolo[1,2-a]pyrazine-6-carboxylate (48 mg, 0.075 mmol), potassium trifluoro(vinyl)borate (17.2 mg, 0.128 mmol), Pd(dppf)Cl₂ (2.9 mg, 0.004 mmol), DIEA (39 μL, 0.23 mmol), isopropanol (0.6 mL), and water (0.3 mL). The mixture was heated at 100° C. for 15 min under microwave irradiation. The reaction mixture was quenched with water and diluted with EtOAc. The organic layer was collected, washed with brine, dried over Na₂SO₄, filtered, and conc in vacuo. The crude material was chromatographed with heptane-EtOAc gradient to afford benzyl (S)-1-chloro-4-oxo-3-(((5-vinyl-[1,1′-biphenyl]-3-yl)methyl)amino)-4,6,7,8-tetrahydropyrrolo[1,2-a]pyrazine-6-carboxylate as a thick oil. The product was used as-is in the subsequent synthetic step.

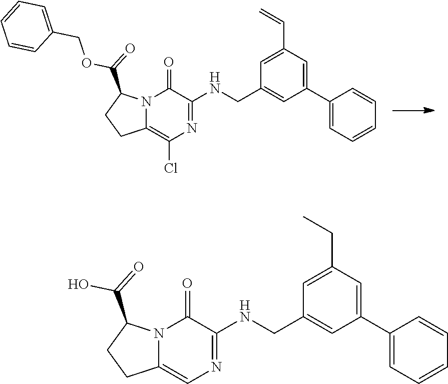 [SMILES: C=Cc1cc(CNc2nc(Cl)c3n(c2=O)[C@H](C(=O)OCc2ccccc2)CC3)cc(-c2ccccc2)c1 ; CCc1cc(CNc2ncc3n(c2=O)[C@H](C(=O)O)CC3)cc(-c2ccccc2)c1]

Step 5: General Hydrogenolysis Procedure A was used to afford (S)-3-(((5-ethyl-[1,1′-biphenyl]-3-yl)methyl)amino)-4-oxo-4,6,7,8-tetrahydropyrrolo[1,2-a]pyrazine-6-carboxylic acid that was used in the subsequent reaction as is.

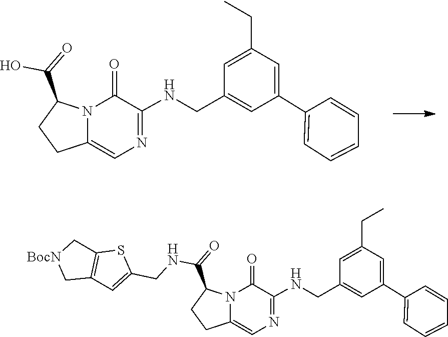 [SMILES: CCc1cc(CNc2ncc3n(c2=O)[C@H](C(=O)O)CC3)cc(-c2ccccc2)c1 ; CCc1cc(CNc2ncc3n(c2=O)[C@H](C(=O)NCc2cc4c(s2)CN(C(=O)OC(C)(C)C)C4)CC3)cc(-c2ccccc2)c1]

Step 6: General Amide Coupling Procedure A was used, except that the crude material was chromatographed with heptane-EtOAc gradient, to afford tert-butyl (S)-2-((3-(((5-ethyl-[1,1′-biphenyl]-3-yl)methyl)amino)-4-oxo-4,6,7,8-tetrahydropyrrolo[1,2-a]pyrazine-6-carboxamido)methyl)-4,6-dihydro-5H-thieno[2,3-c]pyrrole-5-carboxylate (7.1 mg, 23% yield).

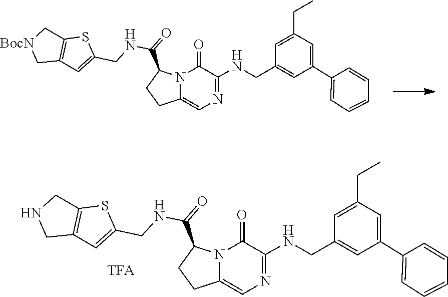 [SMILES: CCc1cc(CNc2ncc3n(c2=O)[C@H](C(=O)NCc2cc4c(s2)CN(C(=O)OC(C)(C)C)C4)CC3)cc(-c2ccccc2)c1 ; CCc1cc(CNc2ncc3n(c2=O)[C@H](C(=O)NCc2cc4c(s2)CNC4)CC3)cc(-c2ccccc2)c1]

Step 7: General Boc Removal Procedure A was used, except that the crude material was purified by prep HPLC, to afford (S)—N-((5,6-dihydro-4H-thieno[2,3-c]pyrrol-2-yl)methyl)-3-(((5-ethyl-[1,1′-biphenyl]-3-yl)methyl)amino)-4-oxo-4,6,7,8-tetrahydropyrrolo[1,2-a]pyrazine-6-carboxamide trifluoroacetate (6.2 mg, 73% yield).

### Example 62

### Preparation of (S)—N-((5,6-dihydro-4H-thieno[2,3-c]pyrrol-2-yl)methyl)-4-oxo-3-(((R)-1-phenylbutyl)amino)-4,6,7,8-tetrahydropyrrolo[1,2-a]pyrazine-6-carboxamide trifluoroacetate

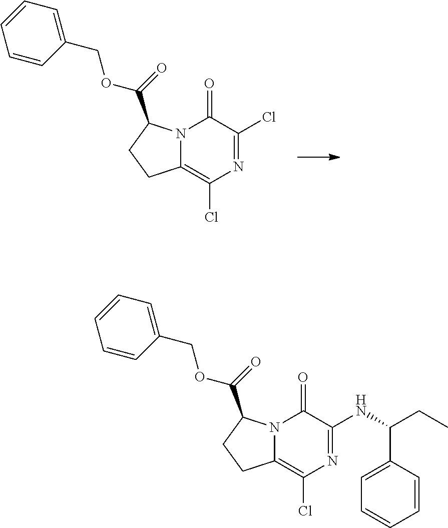 [SMILES: O=C(OCc1ccccc1)[C@@H]1CCc2c(Cl)nc(Cl)c(=O)n21 ; CC[C@@H](Nc1nc(Cl)c2n(c1=O)[C@H](C(=O)OCc1ccccc1)CC2)c1ccccc1]

Step 1: General Chloro Displacement Procedure A was used to afford benzyl (S)-1-chloro-4-oxo-3-(((R)-1-phenylbutyl)amino)-4,6,7,8-tetrahydropyrrolo[1,2-a]pyrazine-6-carboxylate as an off-white foam (116 mg, 61% yield).

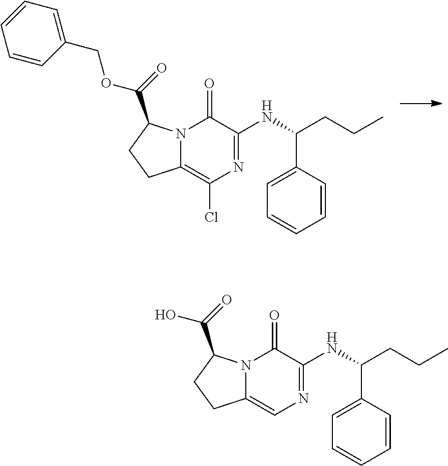 [SMILES: CCC[C@@H](Nc1nc(Cl)c2n(c1=O)[C@H](C(=O)OCc1ccccc1)CC2)c1ccccc1 ; CCC[C@@H](Nc1ncc2n(c1=O)[C@H](C(=O)O)CC2)c1ccccc1]

Step 2: General Hydrogenolysis Procedure A was used to afford (S)-4-oxo-3-(((R)-1-phenylbutyl)amino)-4,6,7,8-tetrahydropyrrolo[1,2-a]pyrazine-6-carboxylic acid (quantitative yield).

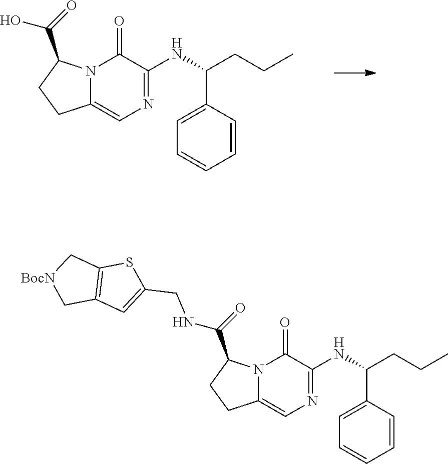 [SMILES: CCC[C@@H](Nc1ncc2n(c1=O)[C@H](C(=O)O)CC2)c1ccccc1 ; CCC[C@@H](Nc1ncc2n(c1=O)[C@H](C(=O)NCc1cc3c(s1)CN(C(=O)OC(C)(C)C)C3)CC2)c1ccccc1]

Step 3: General Amide Coupling Procedure A was used, except that the crude material was chromatographed with heptane-EtOAc gradient, to afford tert-butyl 2-(((S)-4-oxo-3-(((R)-1-phenylbutyl)amino)-4,6,7,8-tetrahydropyrrolo[1,2-a]pyrazine-6-carboxamido)methyl)-4,6-dihydro-5H-thieno[2,3-c]pyrrole-5-carboxylate (96 mg, 66% yield).

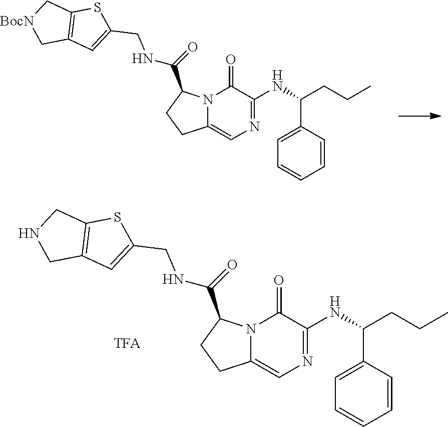 [SMILES: CCC[C@@H](Nc1ncc2n(c1=O)[C@H](C(=O)NCc1cc3c(s1)CN(C(=O)OC(C)(C)C)C3)CC2)c1ccccc1 ; CCC[C@@H](Nc1ncc2n(c1=O)[C@H](C(=O)NCc1cc3c(s1)CNC3)CC2)c1ccccc1]

Step 4: General Boc Removal Procedure A was used, except that the crude material was purified by prep HPLC to afford (S)—N-((5,6-dihydro-4H-thieno[2,3-c]pyrrol-2-yl)methyl)-4-oxo-3-(((R)-1-phenylbutyl)amino)-4,6,7,8-tetrahydropyrrolo[1,2-a]pyrazine-6-carboxamide trifluoroacetate (76 mg, 64% yield).

The following compounds were prepared according to the foregoing procedures using the appropriate amines:

| | Cmp |
|---|---|
| Compound Name | No. |
| (S)-3-(((R)-cyclopropyl(phenyl)methyl)amino)-N-((5,6-dihydro- | 259 |
| 4H-thieno[2,3-c]pyrrol-2-yl)methyl)-4-oxo-4,6,7,8-tetrahydro- | |
| pyrrolo[1,2-a]pyrazine-6-carboxamide trifluoroacetate | |
| (S)-N-((5,6-dihydro-4H-thieno[2,3-c]pyrrol-2-yl)methyl)-4-oxo-3- | 261 |
| ((1-phenylcyclobutyl)amino)-4,6,7,8-tetrahydropyrrolo[1,2- | |
| a]pyrazine-6-carboxamide trifluoroacetate | |
| (S)-N-((5,6-dihydro-4H-thieno[2,3-c]pyrrol-2-yl)methyl)-3-(((R)-1- | 263 |
| (naphthalen-2-yl)propyl)amino)-4-oxo-4,6,7,8-tetrahydro- | |
| pyrrolo[1,2-a]pyrazine-6-carboxamide trifluoroacetate | |
| (S)-N-((5,6-dihydro-4H-thieno[2,3-c]pyrrol-2-yl)methyl)-3-(((R)- | 264 |
| 2,2-dimethyl-1-phenylpropyl)amino)-4-oxo-4,6,7,8-tetrahydro- | |
| pyrrolo[1,2-a]pyrazine-6-carboxamide trifluoroacetate | |
| (S)-N-((5,6-dihydro-4H-thieno[2,3-c]pyrrol-2-yl)methyl)-4-oxo-3- | 265 |
| (((R)-1-(o-tolyl)propyl)amino)-4,6,7,8-tetrahydropyrrolo[1,2- | |
| a]pyrazine-6-carboxamide trifluoroacetate | |
| (S)-N-((5,6-dihydro-4H-thieno[2,3-c]pyrrol-2-yl)methyl)-4-oxo-3- | 266 |
| (((R)-1-(p-tolyl)propyl)amino)-4,6,7,8-tetrahydropyrrolo[1,2- | |
| a]pyrazine-6-carboxamide trifluoroacetate | |
| (S)-3-(((R)-1-([1,1'-biphenyl]-3-yl)propyl)amino)-N-((5,6-dihydro- | 267 |
| 4H-thieno[2,3-c]pyrrol-2-yl)methyl)-4-oxo-4,6,7,8-tetrahydro- | |
| pyrrolo[1,2-a]pyrazine-6-carboxamide trifluoroacetate | |
| (S)-N-((5,6-dihydro-4H-thieno[2,3-c]pyrrol-2-yl)methyl)-3-(((R)-1- | 268 |
| (3-ethylphenyl)propyl)amino)-4-oxo-4,6,7,8-tetrahydropyrrolo[1,2- | |
| a]pyrazine-6-carboxamide trifluoroacetate | |
| (S)-N-((5,6-dihydro-4H-thieno[2,3-c]pyrrol-2-yl)methyl)-4-oxo-3- | 271 |
| (((1-phenylcyclobutyl)methyl)amino)-4,6,7,8-tetrahydro- | |
| pyrrolo[1,2-a]pyrazine-6-carboxamide trifluoroacetate | |
| (S)-N-((5,6-dihydro-4H-thieno[2,3-c]pyrrol-2-yl)methyl)-4-oxo-3- | 276 |
| (((R)-2-phenylpropyl)amino)-4,6,7,8-tetrahydropyrrolo[1,2- | |
| a]pyrazine-6-carboxamide trifluoroacetate | |

### Example 63

### Preparation of (S)—N-((5,6-dihydro-4H-thieno[2,3-c]pyrrol-2-yl)methyl)-3-(((R)-1-(2,5-dimethylphenyl)ethyl)amino)-4-oxo-4,6,7,8-tetrahydropyrrolo[1,2-a]pyrazine-6-carboxamide

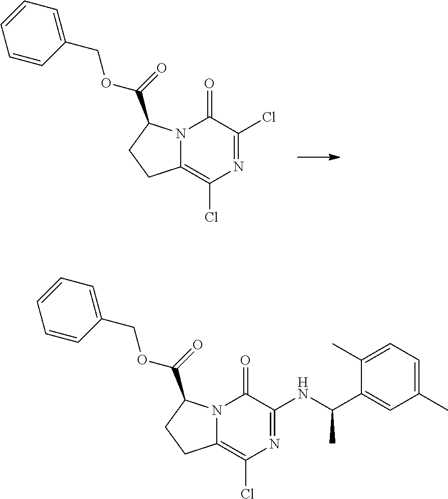 [SMILES: O=C(OCc1ccccc1)[C@@H]1CCc2c(Cl)nc(Cl)c(=O)n21 ; Cc1ccc(C)c([C@@H](C)Nc2nc(Cl)c3n(c2=O)[C@H](C(=O)OCc2ccccc2)CC3)c1]

Step 1: General Chloro Displacement Procedure A was used to afford benzyl (S)-1-chloro-3-(((R)-1-(2,5-dimethylphenyl)ethyl)amino)-4-oxo-4,6,7,8-tetrahydropyrrolo[1,2-a]pyrazine-6-carboxylate as an off-white solid (92 mg, 56% yield.

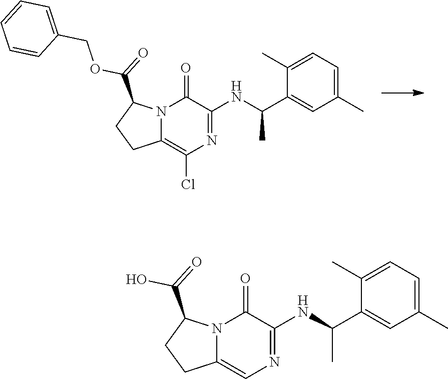 [SMILES: Cc1ccc(C)c([C@@H](C)Nc2nc(Cl)c3n(c2=O)[C@H](C(=O)OCc2ccccc2)CC3)c1 ; Cc1ccc(C)c([C@@H](C)Nc2ncc3n(c2=O)[C@H](C(=O)O)CC3)c1]

Step 2: General Hydrogenolysis Procedure A was used to afford the crude (S)-3-(((R)-1-(2,5-dimethylphenyl)ethyl)amino)-4-oxo-4,6,7,8-tetrahydropyrrolo[1,2-a]pyrazine-6-carboxylic acid.

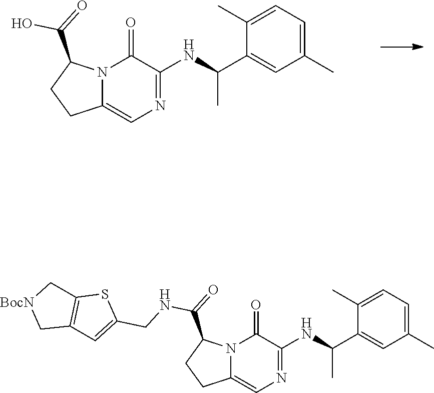 [SMILES: Cc1ccc(C)c([C@@H](C)Nc2ncc3n(c2=O)[C@H](C(=O)O)CC3)c1 ; Cc1ccc(C)c([C@@H](C)Nc2ncc3n(c2=O)[C@H](C(=O)NCc2cc4c(s2)CN(C(=O)OC(C)(C)C)C4)CC3)c1]

Step 3: General Amide Coupling Procedure A was used to afford tert-butyl 2-(((S)-3-(((R)-1-(2,5-dimethylphenyl)ethyl)amino)-4-oxo-4,6,7,8-tetrahydropyrrolo[1,2-a]pyrazine-6-carboxamido)methyl)-4,6-dihydro-5H-thieno[2,3-c]pyrrole-5-carboxylate as a white solid (36 mg, 32% yield).

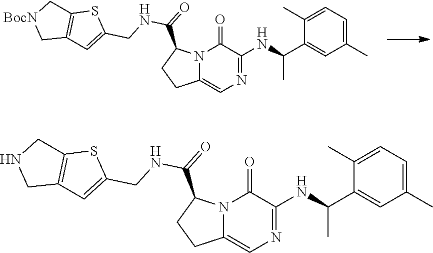 [SMILES: Cc1ccc(C)c([C@@H](C)Nc2ncc3n(c2=O)[C@H](C(=O)NCc2cc4c(s2)CN(C(=O)OC(C)(C)C)C4)CC3)c1 ; Cc1ccc(C)c([C@@H](C)Nc2ncc3n(c2=O)[C@H](C(=O)NCc2cc4c(s2)CNC4)CC3)c1]

Step 4: General Boc Removal Procedure A was used to afford (S)—N-((5,6-dihydro-4H-thieno[2,3-c]pyrrol-2-yl)methyl)-3-(((R)-1-(2,5-dimethylphenyl)ethyl)amino)-4-oxo-4,6,7,8-tetrahydropyrrolo[1,2-a]pyrazine-6-carboxamide as a white solid (12.4 mg, 42% yield).

The following compound was prepared according to the foregoing procedure using the appropriate amine:

| | Cmp |
|---|---|
| Compound Name | No. |
| (S)-3-(((R)-2,3-dihydro-1H-inden-1-yl)amino)-N-((5,6-dihydro-4H- | 254 |
| thieno[2,3-c]pyrrol-2-yl)methyl)-4-oxo-4,6,7,8-tetrahydro- | |
| pyrrolo[1,2-a]pyrazine-6-carboxamide | |

### Example 64

### Preparation of (S)-3-(((R)-cyclopentyl(phenyl)methyl)amino)-N-((5,6-dihydro-4H-thieno[2,3-c]pyrrol-2-yl)methyl)-4-oxo-4,6,7,8-tetrahydropyrrolo[1,2-a]pyrazine-6-carboxamide

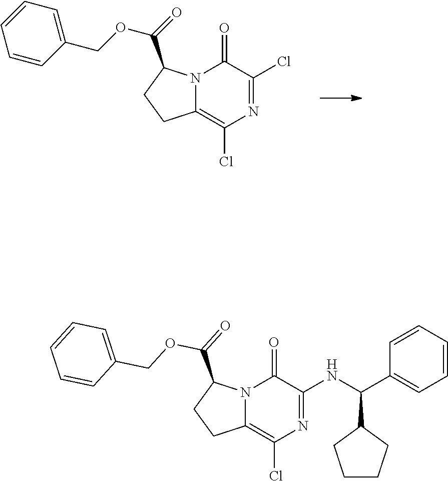 [SMILES: O=C(OCc1ccccc1)[C@@H]1CCc2c(Cl)nc(Cl)c(=O)n21 ; O=C(OCc1ccccc1)[C@@H]1CCc2c(Cl)nc(N[C@@H](c3ccccc3)C3CCCC3)c(=O)n21]

Step 1: A 20 mL vial was charged with benzyl (S)-1,3-dichloro-4-oxo-4,6,7,8-tetrahydropyrrolo[1,2-a]pyrazine-6-carboxylate (100 mg, 295 μmol), (R)-cyclopentyl (phenyl)methanamine hydrochloride (74.9 mg, 354 μmol), DIEA (0.123 mL, 0.708 mmol), and ACN (3.0 mL) to give a solution. The solution was heated up to 90° C. and stirred overnight. The crude reaction was conc in vacuo and chromatographed (0-50% heptane-EtOAc) to afford benzyl (S)-1-chloro-3-(((R)-cyclopentyl(phenyl)methyl)amino)-4-oxo-4,6,7,8-tetrahydropyrrolo[1,2-a]pyrazine-6-carboxylate as a white solid (88 mg, 62% yield).

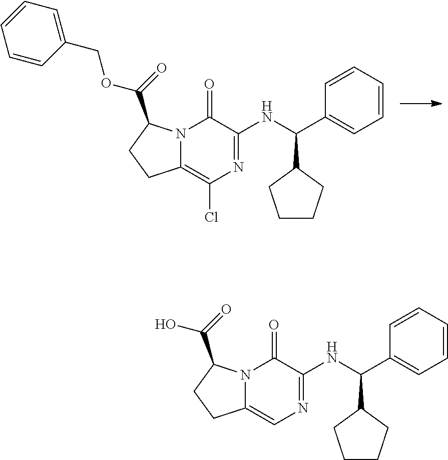 [SMILES: O=C(OCc1ccccc1)[C@@H]1CCc2c(Cl)nc(N[C@@H](c3ccccc3)C3CCCC3)c(=O)n21 ; O=C(O)[C@@H]1CCc2cnc(N[C@@H](c3ccccc3)C3CCCC3)c(=O)n21]

Step 2: Benzyl (S)-1-chloro-3-(((R)-cyclopentyl(phenyl)methyl)amino)-4-oxo-4,6,7,8-tetrahydropyrrolo[1,2-a]pyrazine-6-carboxylate (120 mg, 0.251 mmol) and DIEA (87.5 μL, 0.502 mmol) were dissolved in EtOH (3 mL). The flask was evacuated and backfilled with N₂ three times then Pd/C (13.4 mg, 10% wt) was added. The flask was evacuated and backfilled with H₂ three times then allowed to stir under a H₂ atm for 24 h. The crude material was filtered (PTFE syringe filter) and conc in vacuo to afford an oil quantitative yield).

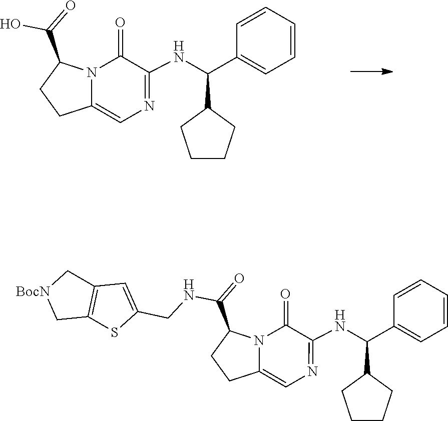 [SMILES: O=C(O)[C@@H]1CCc2cnc(N[C@@H](c3ccccc3)C3CCCC3)c(=O)n21 ; CC(C)(C)OC(=O)N1Cc2cc(CNC(=O)[C@@H]3CCc4cnc(N[C@@H](c5ccccc5)C5CCCC5)c(=O)n43)sc2C1]

Step 3: tert-Butyl 2-(aminomethyl)-4,6-dihydro-5H-thieno[2,3-c]pyrrole-5-carboxylate (44 mg, 0.17 mmol), (S)-3-(((R)-cyclopentyl(phenyl)methyl)amino)-4-oxo-4,6,7,8-tetrahydropyrrolo[1,2-a]pyrazine-6-carboxylic acid (73 mg, 0.21 mmol), HATU (86 mg, 0.22 mmol) and DIEA (90 μL, 0.52 mmol) were dissolved in DMF (0.4 mL). The solution was stirred for 5 h. The crude material was chromatographed (0-50% heptane-EtOAc) to afford tert-butyl 2-(((S)-3-(((R)-cyclopentyl(phenyl)methyl)amino)-4-oxo-4,6,7,8-tetrahydropyrrolo[1,2-a]pyrazine-6-carboxamido)methyl)-4,6-dihydro-5H-thieno[2,3-c]pyrrole-5-carboxylate as a clear solid (42 mg, 42% yield).

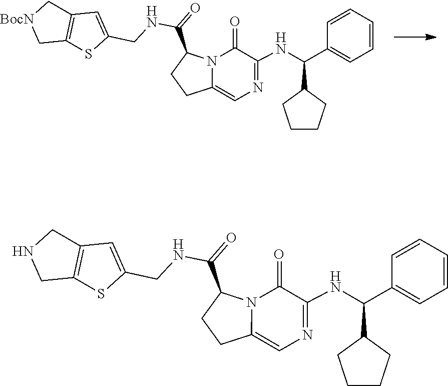 [SMILES: CC(C)(C)OC(=O)N1Cc2cc(CNC(=O)[C@@H]3CCc4cnc(N[C@@H](c5ccccc5)C5CCCC5)c(=O)n43)sc2C1 ; O=C(NCc1cc2c(s1)CNC2)[C@@H]1CCc2cnc(N[C@@H](c3ccccc3)C3CCCC3)c(=O)n21]

Step 4: tert-Butyl 2-(((S)-3-(((R)-cyclopentyl(phenyl)methyl)amino)-4-oxo-4,6,7,8-tetrahydropyrrolo[1,2-a]pyrazine-6-carboxamido)methyl)-4,6-dihydro-5H-thieno[2,3-c]pyrrole-5-carboxylate (45 mg, 76 μmol) and TFA (88 μL, 1.1 mmol) were dissolved in DCM (0.50 mL). The reaction was allowed to stir for 5 h. After conc in vacuo, the product was chromatographed (amine column, MeOH-DCM 0-20% gradient) to afford (S)-3-(((R)-cyclopentyl(phenyl)methyl) amino)-N-((5,6-dihydro-4H-thieno[2,3-c]pyrrol-2-yl)methyl)-4-oxo-4,6,7,8-tetrahydropyrrolo [1,2-a]pyrazine-6-carboxamide as a white solid (16 mg, 43% yield).

### Example 65

### Preparation of (S)—N-((5,6-dihydro-4H-thieno[2,3-c]pyrrol-2-yl)methyl)-3-(((R)-1-(4-fluorophenyl)-2,2-dimethylpropyl)amino)-4-oxo-4,6,7,8-tetrahydropyrrolo[1,2-a]pyrazine-6-carboxamide

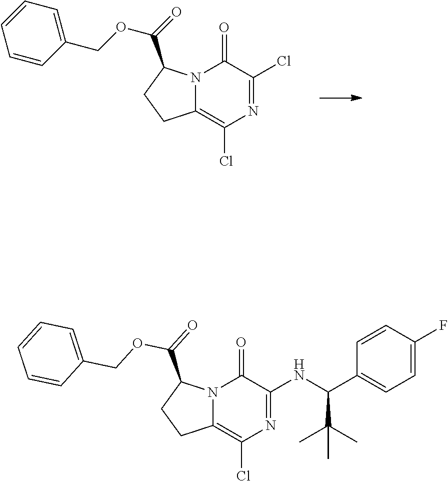 [SMILES: O=C(OCc1ccccc1)[C@@H]1CCc2c(Cl)nc(Cl)c(=O)n21 ; CC(C)(C)[C@@H](Nc1nc(Cl)c2n(c1=O)[C@H](C(=O)OCc1ccccc1)CC2)c1ccc(F)cc1]

Step 1: A 20 mL vial was charged with benzyl (S)-1,3-dichloro-4-oxo-4,6,7,8-tetrahydropyrrolo[1,2-a]pyrazine-6-carboxylate (250 mg, 0.737 mmol), (R)-1-(4-fluorophenyl)-2,2-dimethylpropan-1-amine (160 mg, 0.885 mmol), DIEA (0.180 mL, 1.03 mmol), and ACN (3.0 mL) to give a solution. The reaction was heated up to 90° C. and stirred for 48 h. The solution was conc in vacuo and chromatographed with heptane-EtOAc gradient to afford benzyl (S)-1-chloro-3-(((R)-1-(4-fluorophenyl)-2,2-dimethylpropyl)amino)-4-oxo-4,6,7,8-tetrahydropyrrolo[1,2-a]pyrazine-6-carboxylate as a pale yellow solid (263 mg, 74% yield).

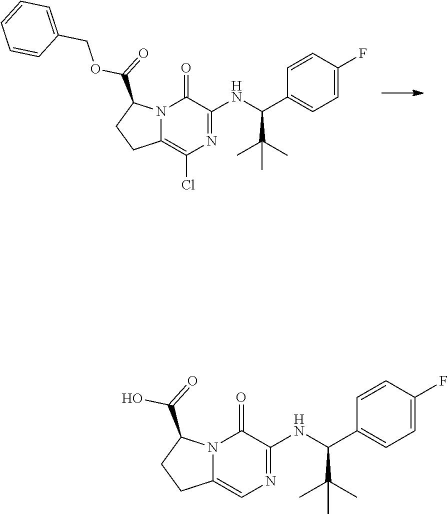 [SMILES: CC(C)(C)[C@@H](Nc1nc(Cl)c2n(c1=O)[C@H](C(=O)OCc1ccccc1)CC2)c1ccc(F)cc1 ; CC(C)(C)[C@@H](Nc1ncc2n(c1=O)[C@H](C(=O)O)CC2)c1ccc(F)cc1]

Step 2: Benzyl (S)-1-chloro-3-(((R)-1-(4-fluorophenyl)-2,2-dimethylpropyl)amino)-4-oxo-4,6,7,8-tetrahydropyrrolo[1,2-a]pyrazine-6-carboxylate (105 mg, 0.217 mmol) was dissolved in EtOH (3 mL). The flask was flushed with N₂ and Pd/C (11.5 mg, 10% wt) was added. The reaction was evacuated and backfilled with H₂ three times. The reaction was stirred for 48 h at which time it was filtered and conc in vacuo to afford (S)-3-(((R)-1-(4-fluorophenyl)-2,2-dimethylpropyl)amino)-4-oxo-4,6,7,8-tetrahydropyrrolo[1,2-a]pyrazine-6-carboxylic acid (quantitative yield).

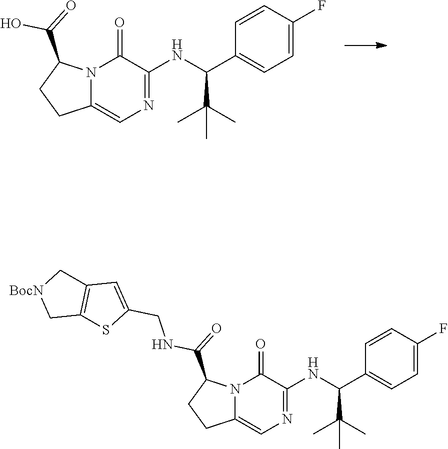 [SMILES: CC(C)(C)[C@@H](Nc1ncc2n(c1=O)[C@H](C(=O)O)CC2)c1ccc(F)cc1 ; CC(C)(C)OC(=O)N1Cc2cc(CNC(=O)[C@@H]3CCc4cnc(N[C@@H](c5ccc(F)cc5)C(C)(C)C)c(=O)n43)sc2C1]

Step 3: (S)-3-(((R)-1-(4-fluorophenyl)-2,2-dimethylpropyl)amino)-4-oxo-4,6,7,8-tetrahydropyrrolo[1,2-a]pyrazine-6-carboxylic acid (40 mg, 0.11 mmol), tert-butyl 2-(aminomethyl)-4,6-dihydro-5H-thieno[2,3-c]pyrrole-5-carboxylate (34 mg, 0.13 mmol), HATU (55 mg, 0.14 mmol), and DIEA (58 μL, 0.33 mmol) were dissolved in DMF (0.4 mL). The reaction was allowed to stir at room temp for 16 h. The crude material was then chromatographed with heptane-EtOAc gradient to afford tert-butyl 2-(((S)-3-(((R)-1-(4-fluorophenyl)-2,2-dimethylpropyl)amino)-4-oxo-4,6,7,8-tetrahydropyrrolo[1,2-a]pyrazine-6-carboxamido)methyl)-4,6-dihydro-5H-thieno[2,3-c]pyrrole-5-carboxylate as a clear oil (60 mg, 90% yield).

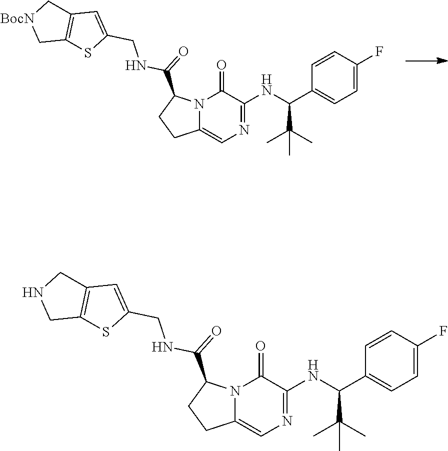 [SMILES: CC(C)(C)OC(=O)N1Cc2cc(CNC(=O)[C@@H]3CCc4cnc(N[C@@H](c5ccc(F)cc5)C(C)(C)C)c(=O)n43)sc2C1 ; CC(C)(C)[C@@H](Nc1ncc2n(c1=O)[C@H](C(=O)NCc1cc3c(s1)CNC3)CC2)c1ccc(F)cc1]

Step 4: tert-Butyl 2-(((S)-3-(((R)-1-(4-fluorophenyl)-2,2-dimethylpropyl)amino)-4-oxo-4,6,7,8-tetrahydropyrrolo[1,2-a]pyrazine-6-carboxamido)methyl)-4,6-dihydro-5H-thieno[2,3-c]pyrrole-5-carboxylate (60 mg, 0.10 mmol) was dissolved in DCM (1 mL) and TFA (0.35 mL, 2.0 mmol) was added. The reaction was allowed to stir overnight. MeOH (3 mL) was added to the mixture then it was conc in vacuo and chromatographed (amine column, 0-20% DCM-MeOH). After conc in vacuo of the product, the residue was re-suspended in ACN-water and lyophilized to afford (S)—N-((5,6-dihydro-4H-thieno[2,3-c]pyrrol-2-yl)methyl)-3-(((R)-1-(4-fluorophenyl)-2,2-dimethylpropyl)amino)-4-oxo-4,6,7,8-tetrahydropyrrolo[1,2-a]pyrazine-6-carboxamide as a white solid (14 mg, 28% yield).

### Example 66

### Preparation of (S)—N-((1H-indazol-5-yl)methyl)-3-((3,5-dimethylbenzyl)amino)-4-oxo-4,6,7,8-tetrahydropyrrolo[1,2-a]pyrazine-6-carboxamide

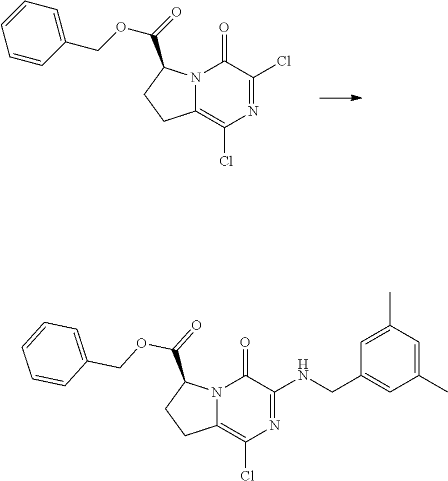 [SMILES: O=C(OCc1ccccc1)[C@@H]1CCc2c(Cl)nc(Cl)c(=O)n21 ; Cc1cc(C)cc(CNc2nc(Cl)c3n(c2=O)[C@H](C(=O)OCc2ccccc2)CC3)c1]

Step 1: Benzyl (S)-1,3-dichloro-4-oxo-4,6,7,8-tetrahydropyrrolo[1,2-a]pyrazine-6-carboxylate (0.300 g, 885 μmol), (3,5-dimethylphenyl)methanamine (132 mg, 0.973 mmol), and sodium acetate (79.8 mg, 0.973 mmol) were suspended in ACN (3.0 mL). The reaction was stirred for 18 h at 90° C. The crude material was conc in vacuo and chromatographed (0-30% heptane-EtOAc) to afford benzyl (S)-1-chloro-3-((3,5-dimethylbenzyl)amino)-4-oxo-4,6,7,8-tetrahydropyrrolo[1,2-a]pyrazine-6-carboxylate (240 mg, 62% yield) as a white solid.

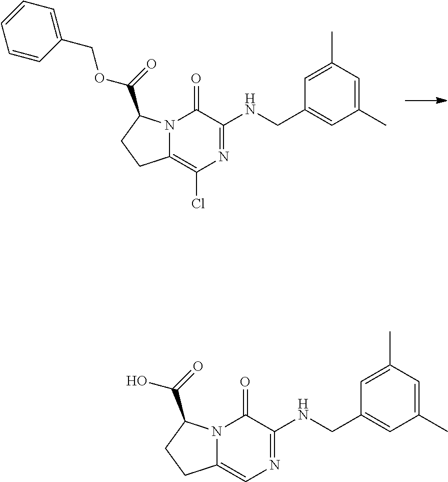 [SMILES: Cc1cc(C)cc(CNc2nc(Cl)c3n(c2=O)[C@H](C(=O)OCc2ccccc2)CC3)c1 ; Cc1cc(C)cc(CNc2ncc3n(c2=O)[C@H](C(=O)O)CC3)c1]

Step 2: Benzyl (S)-1-chloro-3-((3,5-dimethylbenzyl)amino)-4-oxo-4,6,7,8-tetrahydropyrrolo[1,2-a]pyrazine-6-carboxylate (240 mg, 0.548 mmol) was dissolved in EtOH (3 mL). The reaction vessel was flushed with N₂ and Pd/C (29.2 mg, 10% wt) was added. The flask was evacuated and backfilled with H₂ three times. The reaction was allowed to stir for 4 days at which time it was filtered (PTFE syringe filter) and conc in vacuo to afford (S)-3-((3,5-dimethylbenzyl)amino)-4-oxo-4,6,7,8-tetrahydropyrrolo[1,2-a]pyrazine-6-carboxylic acid as a clear oil (quantitative yield).

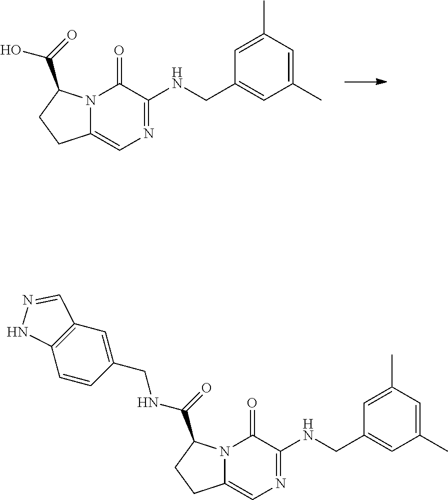 [SMILES: Cc1cc(C)cc(CNc2ncc3n(c2=O)[C@H](C(=O)O)CC3)c1 ; Cc1cc(C)cc(CNc2ncc3n(c2=O)[C@H](C(=O)NCc2ccc4[nH]ncc4c2)CC3)c1]

Step 3: (S)-3-((3,5-dimethylbenzyl)amino)-4-oxo-4,6,7,8-tetrahydropyrrolo[1,2-a]pyrazine-6-carboxylic acid (43 mg, 0.14 mmol), (1H-indazol-5-yl)methanamine (24 mg, 0.16 mmol), HATU (68 mg, 0.18 mmol), and DIEA (72 μL, 0.41 mmol) were dissolved in DMF (1 mL). The reaction was allowed to stir at room temp for 16 h. The material was chromatographed (0-100% heptane-EtOAc) to afford (S)—N-((1H-indazol-5-yl)methyl)-3-((3,5-dimethylbenzyl)amino)-4-oxo-4,6,7,8-tetrahydropyrrolo[1,2-a]pyrazine-6-carboxamide as a white solid (41 mg, 68% yield).

The following compounds were prepared according to the foregoing procedures using the appropriate amines:

| | Cmp |
|---|---|
| Compound Name | No. |
| (S)-N-((1H-indazol-4-yl)methyl)-3-((3,5-dimethylbenzyl)amino)-4- | 282 |
| oxo-4,6,7,8-tetrahydropyrrolo[1,2-a]pyrazine-6-carboxamide | |
| (S)-N-(5-chloro-2-(1H-tetrazol-1-yl)benzyl)-3-((3,5- | 284 |
| dimethylbenzyl)amino)-4-oxo-4,6,7,8-tetrahydropyrrolo[1,2- | |
| a]pyrazine-6-carboxamide | |

### Example 67

### Preparation of (S)—N—((R/S)-1-(5-chloro-2-(1H-tetrazol-1-yl)phenyl)ethyl)-3-((3,5-dimethylbenzyl)amino)-4-oxo-4,6,7,8-tetrahydropyrrolo[1,2-a]pyrazine-6-carboxamide

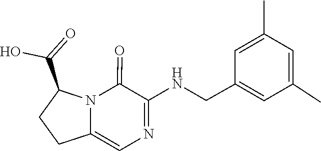 [SMILES: Cc1cc(C)cc(CNc2ncc3n(c2=O)[C@H](C(=O)O)CC3)c1]

Step 1: The above compound was synthesized as described in steps 1 and 2 of Example 66.

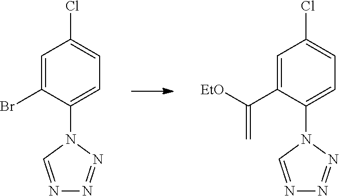 [SMILES: Clc1ccc(-n2cnnn2)c(Br)c1 ; C=C(OCC)c1cc(Cl)ccc1-n1cnnn1]

Step 2: To a solution of 1-(2-bromo-4-chlorophenyl)-1H-tetrazole (200 mg, 771 mol) and Pd(Ph₃P)₄ (44.5 mg, 38.5 μmol) in 1,4-dioxane (2 mL) was added tributyl(1-ethoxyvinyl)stannane (334 mg, 0.925 mmol). The solution was purged with Ar then allowed to stir overnight at 100° C. After cooling to room temp, diethyl ether (100 mL) was added and the solution was washed with 1 M aq. potassium fluoride (2×50 mL) and saturated aq. ammonium chloride, dried over sodium sulfate, and conc in vacuo. Chromatography (0-30% heptane-EtOAc) afforded 1-(4-chloro-2-(1-ethoxyvinyl)phenyl)-1H-tetrazole as an oil (126.5 mg, 66% yield).

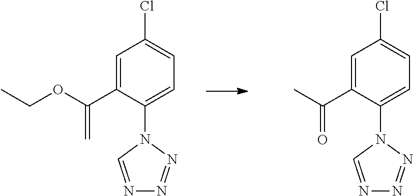 [SMILES: C=C(OCC)c1cc(Cl)ccc1-n1cnnn1 ; CC(=O)c1cc(Cl)ccc1-n1cnnn1]

Step 3: 1-(4-Chloro-2-(1-ethoxyvinyl)phenyl)-1H-tetrazole (126 mg, 0.503 mmol) was dissolved in MeOH (2.0 mL). Hydrochloric acid (conc, 415 μL, 5.03 mmol) was added and the mixture was allowed to stir at room temp for 1 h. The reaction was quenched with sat. aq. NaHCO₃ (50 mL). The aqueous layer was extracted with diethyl ether (3×50 mL), dried over sodium sulfate, and conc in vacuo to afford 1-(5-chloro-2-(1H-tetrazol-1-yl)phenyl)ethan-1-one as a white solid (81.8 mg, 73% yield).

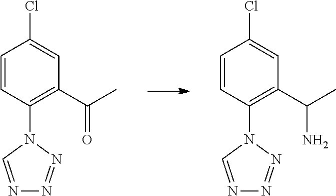

Step 4: 1-(5-Chloro-2-(1H-tetrazol-1-yl)phenyl)ethan-1-one (88 mg, 0.40 mmol), ammonium acetate (0.46 g, 5.9 mmol), and sodium cyanoborohydride (30 mg, 0.47 mmol) were added to EtOH (1 mL). The mixture was heated under microwave irradiation for 2 min at 130° C. After cooling to room temp, the mixture was poured into 2 N NaOH (3 mL) and extracted with EtOAc. The organic layers were combined, dried over sodium sulfate, and conc in vacuo. The crude material was chromatographed (amine column, 50-100% heptane-EtOAc) to afford 1-(5-chloro-2-(1H-tetrazol-1-yl)phenyl)ethan-1-amine as an oil (40 mg, 45% yield).

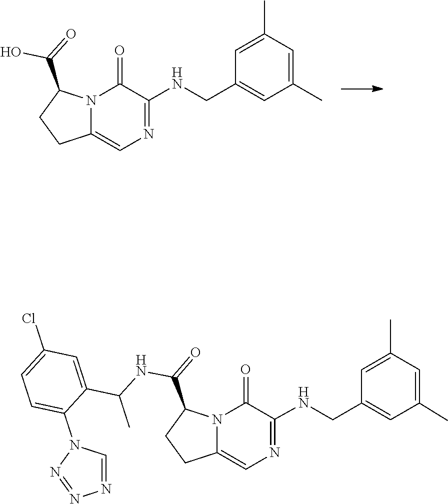 [SMILES: Cc1cc(C)cc(CNc2ncc3n(c2=O)[C@H](C(=O)O)CC3)c1 ; Cc1cc(C)cc(CNc2ncc3n(c2=O)[C@H](C(=O)NC(C)c2cc(Cl)ccc2-n2cnnn2)CC3)c1]

Step 5: (S)-3-((3,5-dimethylbenzyl)amino)-4-oxo-4,6,7,8-tetrahydropyrrolo[1,2-a]pyrazine-6-carboxylic acid (40 mg, 0.13 mmol), 1-(5-chloro-2-(1H-tetrazol-1-yl)phenyl)ethan-1-amine (34 mg, 0.15 mmol), HATU (63 mg, 0.17 mmol), and DIEA (67 L, 0.38 mmol) were dissolved in DMF (0.4 mL). The reaction was allowed to stir at room temp for 16 h. Purification by prep HPLC afforded (S)—N—((R/S)-1-(5-chloro-2-(1H-tetrazol-1-yl)phenyl)ethyl)-3-((3,5-dimethylbenzyl)amino)-4-oxo-4,6,7,8-tetrahydropyrrolo[1,2-a]pyrazine-6-carboxamide as a white solid (38 mg, 57%).

### Example 68

### Preparation of (S)-3-(([1,1′-biphenyl]-3-ylmethyl)amino)-N-((5,6-dihydro-4H-thieno[2,3-c]pyrrol-2-yl)methyl)-4-oxo-4,6,7,8-tetrahydropyrrolo[1,2-a]pyrazine-6-carboxamide trifluoroacetate

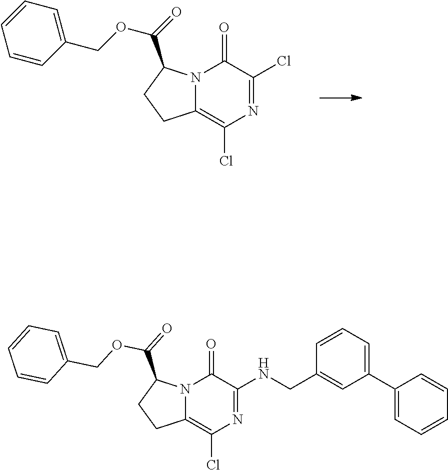 [SMILES: O=C(OCc1ccccc1)[C@@H]1CCc2c(Cl)nc(Cl)c(=O)n21 ; O=C(OCc1ccccc1)[C@@H]1CCc2c(Cl)nc(NCc3cccc(-c4ccccc4)c3)c(=O)n21]

Step 1: To a solution of benzyl (S)-1,3-dichloro-4-oxo-4,6,7,8-tetrahydropyrrolo[1,2-a]pyrazine-6-carboxylate (100 mg, 295 μmol), and [1,1′-biphenyl]-3-ylmethanamine (54.0 mg, 295 μmol) in ACN (2.0 mL) was added DIEA (154 μL, 885 mol) under N₂. The clear solution was heated at 85° C. for 16 h. Volatiles were then removed in vacuo. The crude product was chromatographed (0-70% EtOAc-hexane) to afford benzyl (S)-3-(([1,1′-biphenyl]-3-ylmethyl)amino)-1-chloro-4-oxo-4,6,7,8-tetrahydropyrrolo[1,2-a]pyrazine-6-carboxylate (120 mg, 84%) as an off-white sticky solid.

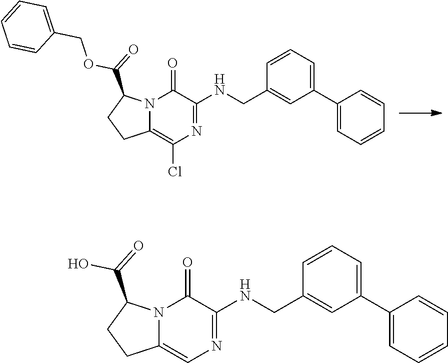 [SMILES: O=C(OCc1ccccc1)[C@@H]1CCc2c(Cl)nc(NCc3cccc(-c4ccccc4)c3)c(=O)n21 ; O=C(O)[C@@H]1CCc2cnc(NCc3cccc(-c4ccccc4)c3)c(=O)n21]

Step 2: MeOH (4 mL) was added to benzyl (S)-3-(([1,1′-biphenyl]-3-ylmethyl)amino)-1-chloro-4-oxo-4,6,7,8-tetrahydropyrrolo[1,2-a]pyrazine-6-carboxylate (120 mg, 247 μmol). The mixture was sonicated for 5 min. The cloudy mixture was purged with N₂, then Pd/C (13.1 mg, 10% wt.) was added. The reaction flask was evacuated, H₂ was introduced, then the mixture was stirred under H₂ for 18 h. The mixture was filtered through a pad of diatomaceous earth, and conc in vacuo to afford (S)-3-(([1,1′-biphenyl]-3-ylmethyl)amino)-4-oxo-4,6,7,8-tetrahydropyrrolo[1,2-a]pyrazine-6-carboxylic acid (85 mg, 95%).

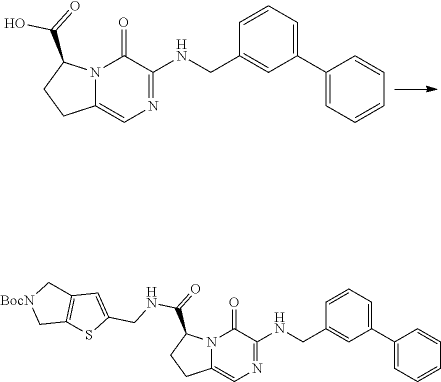 [SMILES: O=C(O)[C@@H]1CCc2cnc(NCc3cccc(-c4ccccc4)c3)c(=O)n21 ; CC(C)(C)OC(=O)N1Cc2cc(CNC(=O)[C@@H]3CCc4cnc(NCc5cccc(-c6ccccc6)c5)c(=O)n43)sc2C1]

Step 3: To a solution of (S)-3-(([1,1′-biphenyl]-3-ylmethyl)amino)-4-oxo-4,6,7,8-tetrahydropyrrolo[1,2-a]pyrazine-6-carboxylic acid (50 mg, 0.14 mmol) in DMF (1.5 mL) was added DIEA (96 μL, 0.55 mmol) and tert-butyl 2-(aminomethyl)-4,6-dihydro-5H-thieno[2,3-c]pyrrole-5-carboxylate (39 mg, 0.15 mmol). The mixture was cooled to 0° C. then HATU (0.11 g, 0.28 mmol) was added. After stirring for 16 h at room temp, volatiles were removed in vacuo. The residue was chromatographed with 0-100% heptane-EtOAc to afford tert-butyl (S)-2-((3-(([1,1′-biphenyl]-3-ylmethyl)amino)-4-oxo-4,6,7,8-tetrahydropyrrolo[1,2-a]pyrazine-6-carboxamido)methyl)-4,6-dihydro-5H-thieno[2,3-c]pyrrole-5-carboxylate (50 mg, 60% yield).

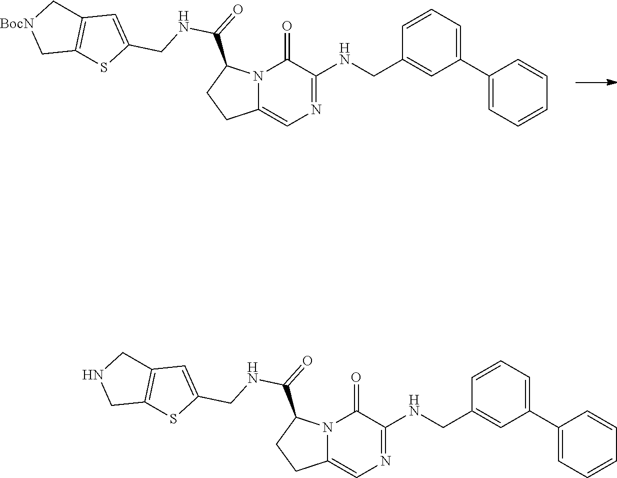 [SMILES: CC(C)(C)OC(=O)N1Cc2cc(CNC(=O)[C@@H]3CCc4cnc(NCc5cccc(-c6ccccc6)c5)c(=O)n43)sc2C1 ; O=C(NCc1cc2c(s1)CNC2)[C@@H]1CCc2cnc(NCc3cccc(-c4ccccc4)c3)c(=O)n21]

Step 4: To a solution of tert-butyl (S)-2-((3-(([1,1′-biphenyl]-3-ylmethyl)amino)-4-oxo-4,6,7,8-tetrahydropyrrolo[1,2-a]pyrazine-6-carboxamido)methyl)-4,6-dihydro-5H-thieno[2,3-c]pyrrole-5-carboxylate (50 mg, 84 μmol) in DCM (1.0 mL) was added TFA (0.13 mL, 1.7 mmol) at room temp. The reaction mixture was stirred at room temp for 18 h. After conc in vacuo, the residue was purified by prep HPLC to provide (S)-3-(([1,1′-biphenyl]-3-ylmethyl)amino)-N-((5,6-dihydro-4H-thieno[2,3-c]pyrrol-2-yl)methyl)-4-oxo-4,6,7,8-tetrahydropyrrolo[1,2-a]pyrazine-6-carboxamide trifluoroacetate as a white solid (28 mg, 55%).

The following compound was synthesized according to the foregoing procedures using the appropriate amine:

| | Cmp |
|---|---|
| Compound Name | No. |
| (S)-N-((5,6-dihydro-4H-thieno[2,3-c]pyrrol-2-yl)methyl)-3-((2- | 286 |
| methylbenzyl)amino)-4-oxo-4,6,7,8-tetrahydropyrrolo[1,2- | |
| a]pyrazine-6-carboxamide trifluoroacetate | |

### Example 69

### Additional Compounds

Compound numbers 234 to 251, 255, and 256 were synthesized by procedures analogous to the procedures described in the foregoing examples, and were evaluated by the procedures described in the examples which follow.

### Example 70

### Exemplary Compounds

The exemplary compounds of the disclosure, as disclosed herein and produced by the Examples above, are shown in Table 1, below.

TABLE 1
Exemplary Compounds of the Disclosure

| | | | Exact | |
|---|---|---|---|---|
| | | | Obs'd. | |
| | | | Mass | Exact |
| Cmp | | | (ES+; | Calc. |
| No. | Structure | Salt | M + H) | Mass |
| 1 | 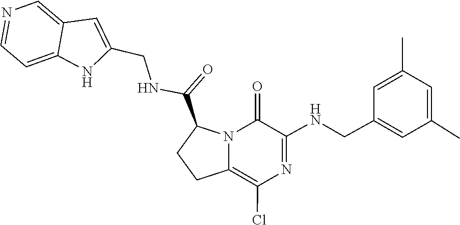 [SMILES: Cc1cc(C)cc(CNc2nc(Cl)c3n(c2=O)[C@H](C(=O)NCc2cc4cnccc4[nH]2)CC3)c1] | | 477.48 | 476.17 |
| 2 | 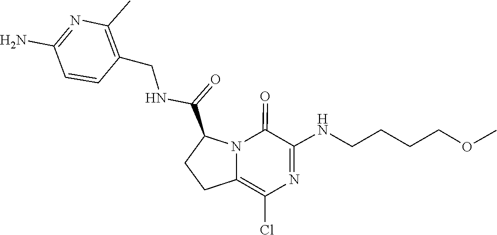 [SMILES: COCCCCNc1nc(Cl)c2n(c1=O)[C@H](C(=O)NCc1ccc(N)nc1C)CC2] | | 435.39 | 434.18 |
| 3 | 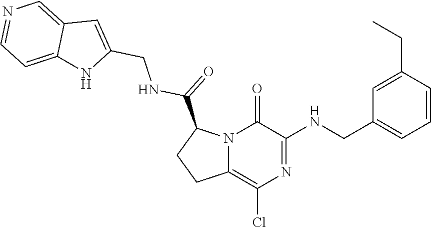 [SMILES: CCc1cccc(CNc2nc(Cl)c3n(c2=O)[C@H](C(=O)NCc2cc4cnccc4[nH]2)CC3)c1] | | 477.48 | 476.17 |
| 4 | 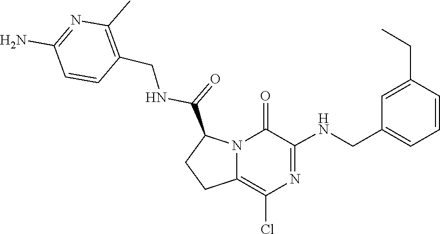 [SMILES: CCc1cccc(CNc2nc(Cl)c3n(c2=O)[C@H](C(=O)NCc2ccc(N)nc2C)CC3)c1] | | 467.50 | 466.19 |
| 5 | 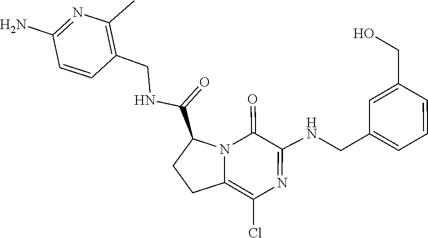 [SMILES: Cc1nc(N)ccc1CNC(=O)[C@@H]1CCc2c(Cl)nc(NCc3cccc(CO)c3)c(=O)n21] | | 469.43 | 468.17 |
| 6 | 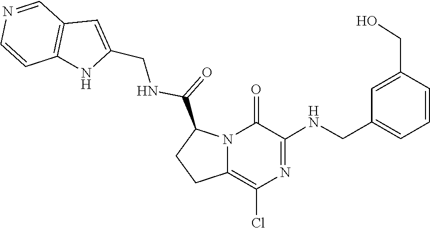 [SMILES: O=C(NCc1cc2cnccc2[nH]1)[C@@H]1CCc2c(Cl)nc(NCc3cccc(CO)c3)c(=O)n21] | | 479.40 | 478.15 |
| 7 | 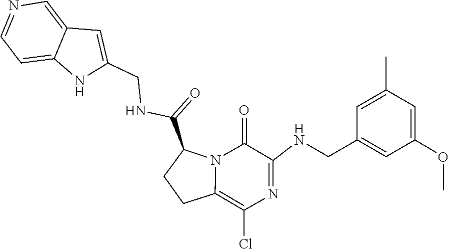 [SMILES: COc1cc(C)cc(CNc2nc(Cl)c3n(c2=O)[C@H](C(=O)NCc2cc4cnccc4[nH]2)CC3)c1] | | 493.49 | 492.17 |
| 8 | 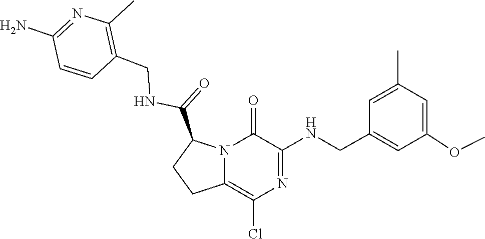 [SMILES: COc1cc(C)cc(CNc2nc(Cl)c3n(c2=O)[C@H](C(=O)NCc2ccc(N)nc2C)CC3)c1] | | 483.51 | 482.18 |
| 9 | 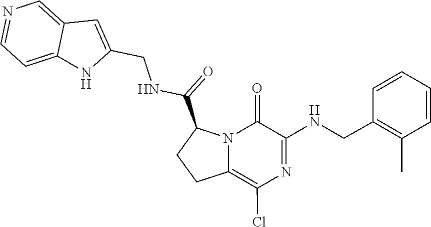 [SMILES: Cc1ccccc1CNc1nc(Cl)c2n(c1=O)[C@H](C(=O)NCc1cc3cnccc3[nH]1)CC2] | | 463.39 | 462.16 |
| 10 | 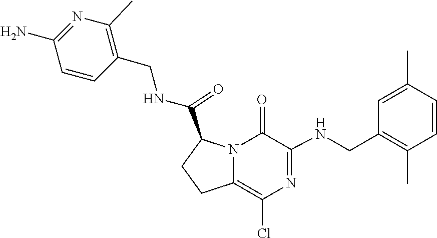 [SMILES: Cc1ccc(C)c(CNc2nc(Cl)c3n(c2=O)[C@H](C(=O)NCc2ccc(N)nc2C)CC3)c1] | | 467.42 | 466.19 |
| 11 | 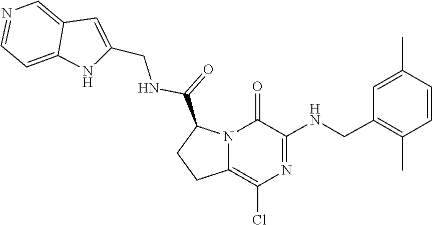 [SMILES: Cc1ccc(C)c(CNc2nc(Cl)c3n(c2=O)[C@H](C(=O)NCc2cc4cnccc4[nH]2)CC3)c1] | | 467.42 | 466.19 |
| 12 | 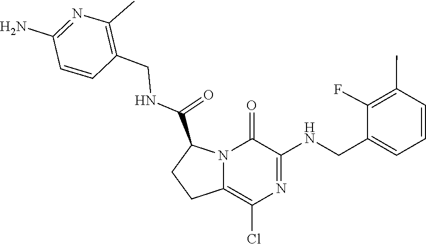 [SMILES: Cc1cccc(CNc2nc(Cl)c3n(c2=O)[C@H](C(=O)NCc2ccc(N)nc2C)CC3)c1F] | | 471.44 | 470.16 |
| 13 | 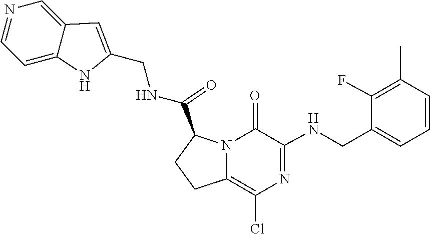 [SMILES: Cc1cccc(CNc2nc(Cl)c3n(c2=O)[C@H](C(=O)NCc2cc4cnccc4[nH]2)CC3)c1F] | | 481.41 | 480.15 |
| 14 | 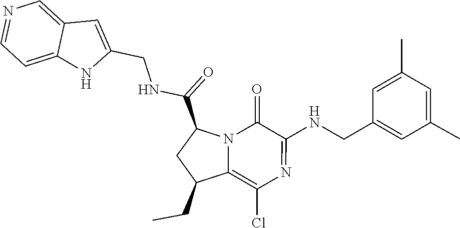 [SMILES: CC[C@H]1C[C@@H](C(=O)NCc2cc3cnccc3[nH]2)n2c1c(Cl)nc(NCc1cc(C)cc(C)c1)c2=O] | | 505.47 | 504.20 |
| 15 | 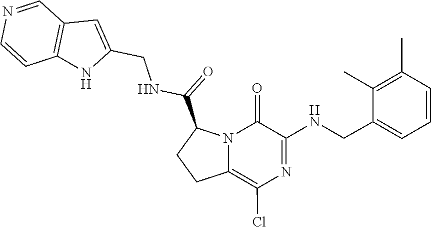 [SMILES: Cc1cccc(CNc2nc(Cl)c3n(c2=O)[C@H](C(=O)NCc2cc4cnccc4[nH]2)CC3)c1C] | | 477.48 | 476.17 |
| 16 | 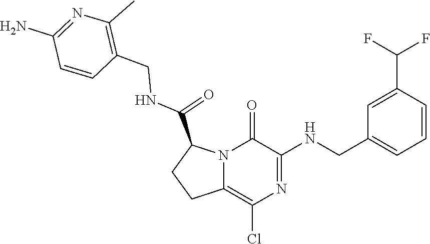 [SMILES: Cc1nc(N)ccc1CNC(=O)[C@@H]1CCc2c(Cl)nc(NCc3cccc(C(F)F)c3)c(=O)n21] | | 489.46 | 488.15 |
| 17 | 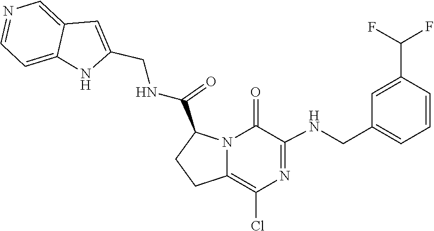 [SMILES: O=C(NCc1cc2cnccc2[nH]1)[C@@H]1CCc2c(Cl)nc(NCc3cccc(C(F)F)c3)c(=O)n21] | | 499.44 | 498.14 |
| 18 | 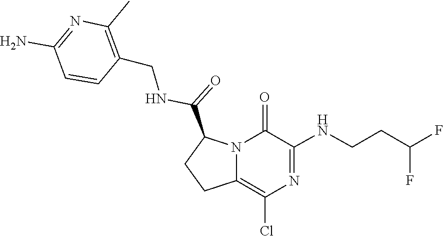 [SMILES: Cc1nc(N)ccc1CNC(=O)[C@@H]1CCc2c(Cl)nc(NCCC(F)F)c(=O)n21] | | 427.43 | 426.14 |
| 19 | 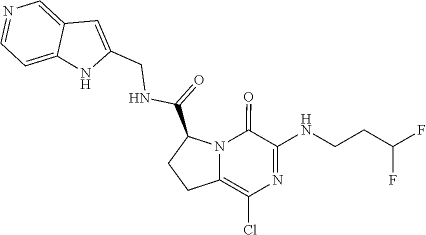 [SMILES: O=C(NCc1cc2cnccc2[nH]1)[C@@H]1CCc2c(Cl)nc(NCCC(F)F)c(=O)n21] | | 437.41 | 436.12 |
| 20 | 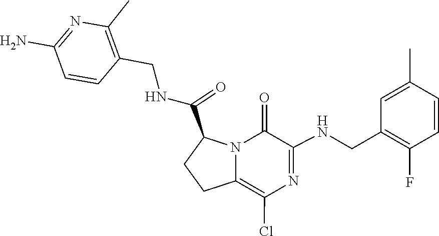 [SMILES: Cc1ccc(F)c(CNc2nc(Cl)c3n(c2=O)[C@H](C(=O)NCc2ccc(N)nc2C)CC3)c1] | | 471.35 | 470.16 |
| 21 | 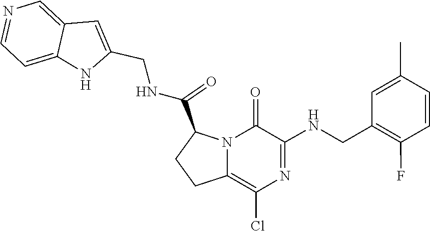 [SMILES: Cc1ccc(F)c(CNc2nc(Cl)c3n(c2=O)[C@H](C(=O)NCc2cc4cnccc4[nH]2)CC3)c1] | | 481.41 | 480.15 |
| 22 | 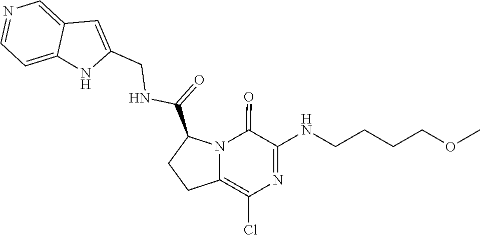 [SMILES: COCCCCNc1nc(Cl)c2n(c1=O)[C@H](C(=O)NCc1cc3cnccc3[nH]1)CC2] | | 445.37 | 444.17 |
| 23 | 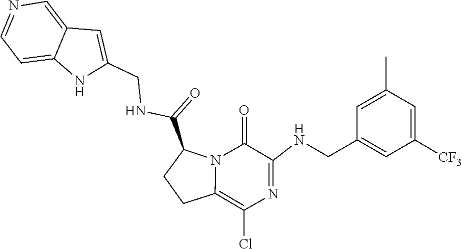 [SMILES: Cc1cc(CNc2nc(Cl)c3n(c2=O)[C@H](C(=O)NCc2cc4cnccc4[nH]2)CC3)cc(C(F)(F)F)c1] | | 531.46 | 530.14 |
| 24 | 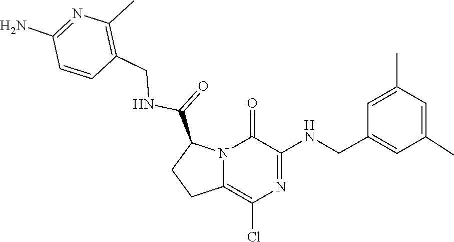 [SMILES: Cc1cc(C)cc(CNc2nc(Cl)c3n(c2=O)[C@H](C(=O)NCc2ccc(N)nc2C)CC3)c1] | | 467.12 | 466.19 |
| 25 | 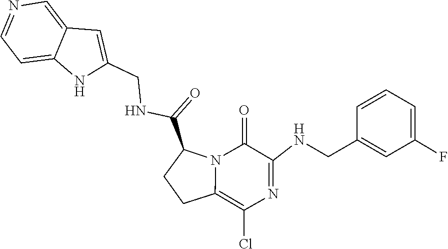 [SMILES: O=C(NCc1cc2cnccc2[nH]1)[C@@H]1CCc2c(Cl)nc(NCc3cccc(F)c3)c(=O)n21] | | 467.33 | 466.13 |
| 26 | 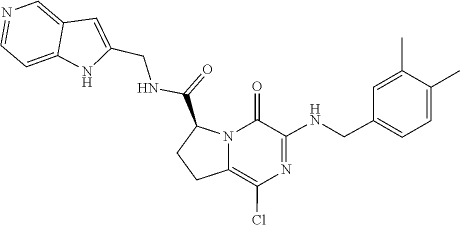 [SMILES: Cc1ccc(CNc2nc(Cl)c3n(c2=O)[C@H](C(=O)NCc2cc4cnccc4[nH]2)CC3)cc1C] | | 477.48 | 476.17 |
| 27 | 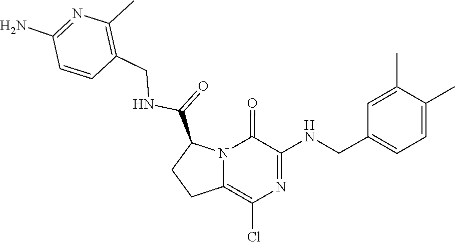 [SMILES: Cc1ccc(CNc2nc(Cl)c3n(c2=O)[C@H](C(=O)NCc2ccc(N)nc2C)CC3)cc1C] | TFA | 467.50 | 466.19 |
| 28 | 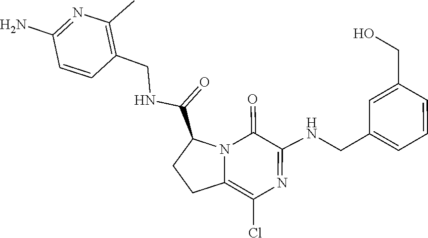 [SMILES: Cc1nc(N)ccc1CNC(=O)[C@@H]1CCc2c(Cl)nc(NCc3cccc(CO)c3)c(=O)n21] | TFA | 453.42 | 452.17 |
| 29 | 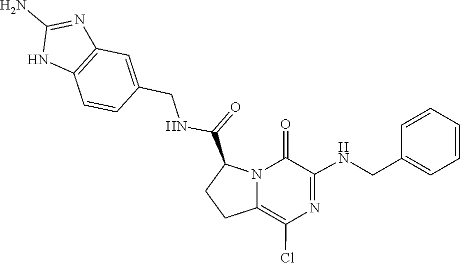 [SMILES: Nc1nc2cc(CNC(=O)[C@@H]3CCc4c(Cl)nc(NCc5ccccc5)c(=O)n43)ccc2[nH]1] | TFA | 464.31 | 463.15 |
| 30 | 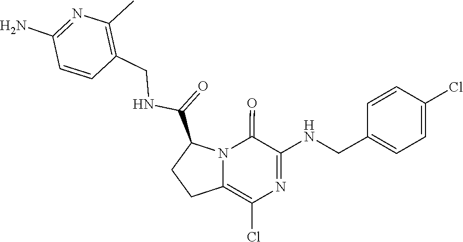 [SMILES: Cc1nc(N)ccc1CNC(=O)[C@@H]1CCc2c(Cl)nc(NCc3ccc(Cl)cc3)c(=O)n21] | TFA | 473.29 | 472.12 |
| 31 | 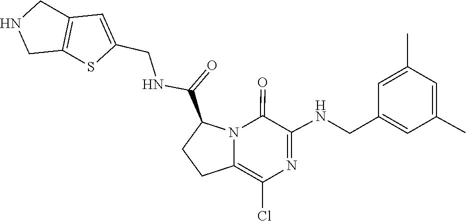 [SMILES: Cc1cc(C)cc(CNc2nc(Cl)c3n(c2=O)[C@H](C(=O)NCc2cc4c(s2)CNC4)CC3)c1] | | 484.48 | 483.15 |
| 32 | 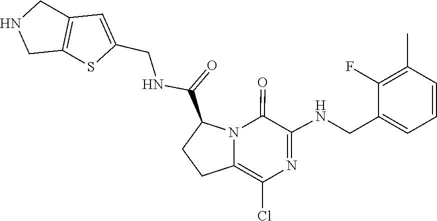 [SMILES: Cc1cccc(CNc2nc(Cl)c3n(c2=O)[C@H](C(=O)NCc2cc4c(s2)CNC4)CC3)c1F] | | 488.41 | 487.12 |
| 33 | 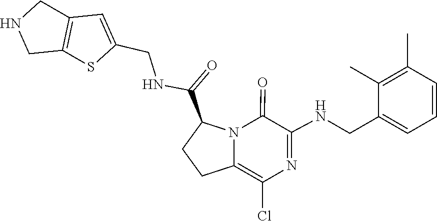 [SMILES: Cc1cccc(CNc2nc(Cl)c3n(c2=O)[C@H](C(=O)NCc2cc4c(s2)CNC4)CC3)c1C] | | 484.48 | 483.15 |
| 34 | 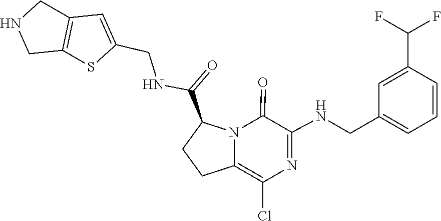 [SMILES: O=C(NCc1cc2c(s1)CNC2)[C@@H]1CCc2c(Cl)nc(NCc3cccc(C(F)F)c3)c(=O)n21] | | 506.44 | 505.12 |
| 35 | 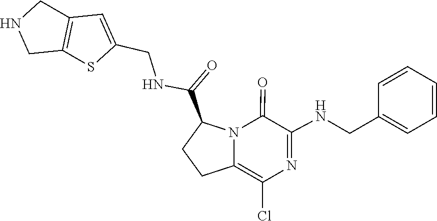 [SMILES: O=C(NCc1cc2c(s1)CNC2)[C@@H]1CCc2c(Cl)nc(NCc3ccccc3)c(=O)n21] | | 456.28 | 455.12 |
| 36 | 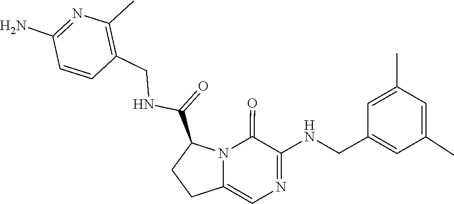 [SMILES: Cc1cc(C)cc(CNc2ncc3n(c2=O)[C@H](C(=O)NCc2ccc(N)nc2C)CC3)c1] | | 433.56 | 432.23 |
| 37 | 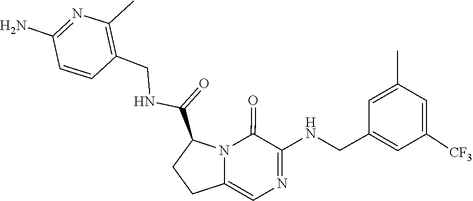 [SMILES: Cc1cc(CNc2ncc3n(c2=O)[C@H](C(=O)NCc2ccc(N)nc2C)CC3)cc(C(F)(F)F)c1] | | 487.45 | 486.2 |
| 38 | 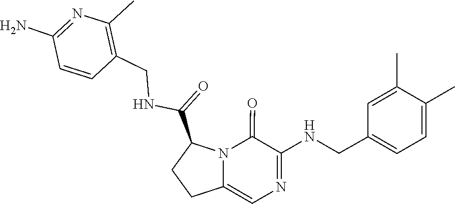 [SMILES: Cc1ccc(CNc2ncc3n(c2=O)[C@H](C(=O)NCc2ccc(N)nc2C)CC3)cc1C] | | 433.56 | 432.23 |
| 39 | 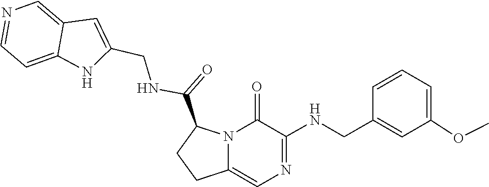 [SMILES: COc1cccc(CNc2ncc3n(c2=O)[C@H](C(=O)NCc2cc4cnccc4[nH]2)CC3)c1] | TFA | 445.46 | 444.19 |
| 40 | 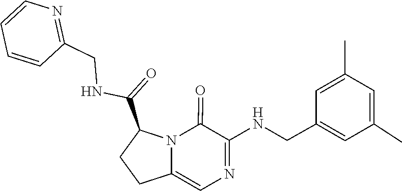 [SMILES: Cc1cc(C)cc(CNc2ncc3n(c2=O)[C@H](C(=O)NCc2ccccn2)CC3)c1] | TFA | 404.16 | 403.20 |
| 41 | 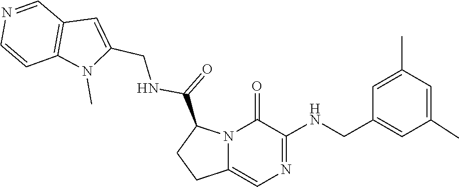 [SMILES: Cc1cc(C)cc(CNc2ncc3n(c2=O)[C@H](C(=O)NCc2cc4cnccc4n2C)CC3)c1] | TFA | 457.17 | 456.23 |
| 42 | 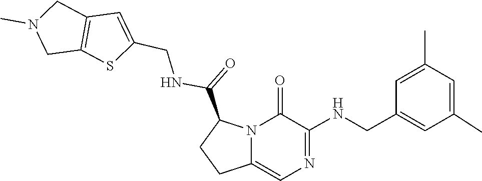 | TFA | 464.16 | 463.20 |
| 43 | 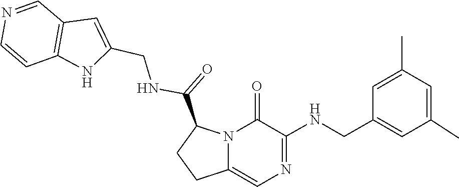 [SMILES: Cc1cc(C)cc(CNc2ncc3n(c2=O)[C@H](C(=O)NCc2cc4cnccc4[nH]2)CC3)c1] | TFA | 457.65 | 456.23 |
| 44 | 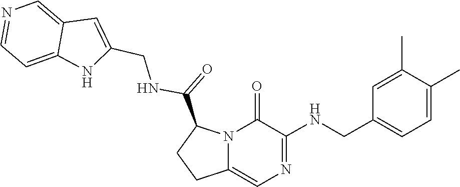 [SMILES: Cc1ccc(CNc2ncc3n(c2=O)[C@H](C(=O)NCc2cc4cnccc4[nH]2)CC3)cc1C] | | 443.53 | 442.21 |
| 45 | 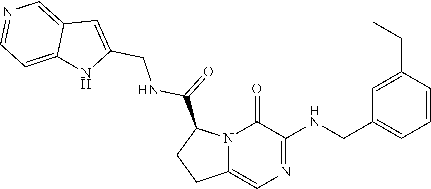 [SMILES: CCc1cccc(CNc2ncc3n(c2=O)[C@H](C(=O)NCc2cc4cnccc4[nH]2)CC3)c1] | | 443.53 | 442.21 |
| 46 | 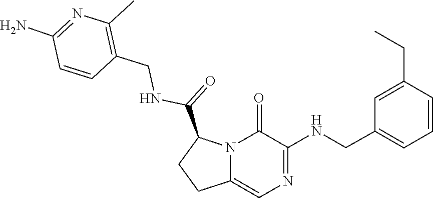 [SMILES: CCc1cccc(CNc2ncc3n(c2=O)[C@H](C(=O)NCc2ccc(N)nc2C)CC3)c1] | | 433.56 | 442.23 |
| 47 | 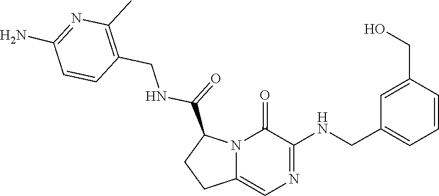 [SMILES: Cc1nc(N)ccc1CNC(=O)[C@@H]1CCc2cnc(NCc3cccc(CO)c3)c(=O)n21] | | 435.48 | 434.21 |
| 48 | 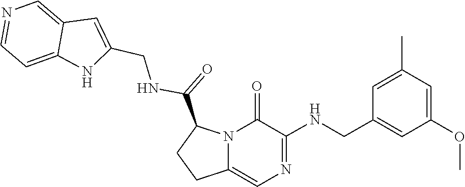 [SMILES: COc1cc(C)cc(CNc2ncc3n(c2=O)[C@H](C(=O)NCc2cc4cnccc4[nH]2)CC3)c1] | | 459.45 | 458.21 |
| 49 | 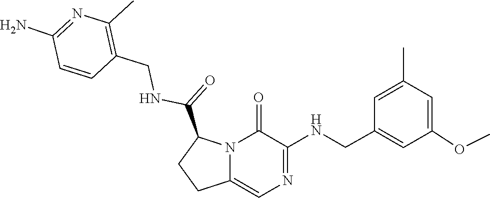 [SMILES: COc1cc(C)cc(CNc2ncc3n(c2=O)[C@H](C(=O)NCc2ccc(N)nc2C)CC3)c1] | | 449.48 | 448.22 |
| 50 | 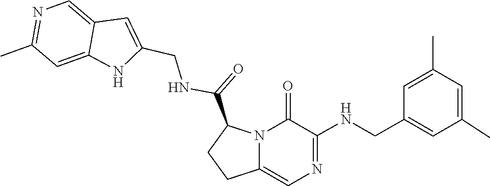 | | 457.53 | 456.23 |
| 51 | 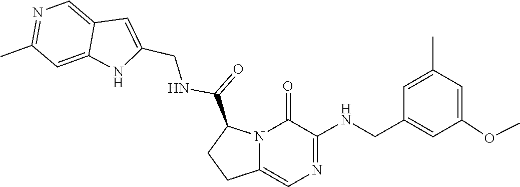 | | 473.54 | 472.22 |
| 52 | 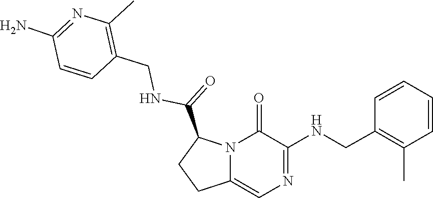 [SMILES: Cc1ccccc1CNc1ncc2n(c1=O)[C@H](C(=O)NCc1ccc(N)nc1C)CC2] | | 419.47 | 418.21 |
| 53 | 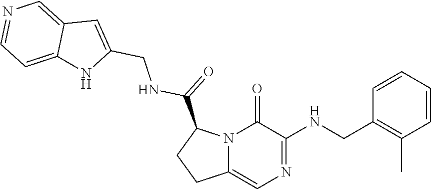 [SMILES: Cc1ccccc1CNc1ncc2n(c1=O)[C@H](C(=O)NCc1cc3cnccc3[nH]1)CC2] | | 429.53 | 428.20 |
| 54 | 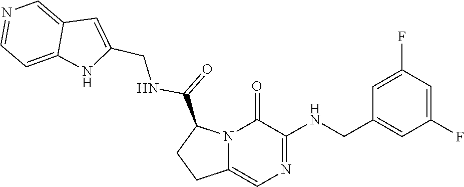 [SMILES: O=C(NCc1cc2cnccc2[nH]1)[C@@H]1CCc2cnc(NCc3cc(F)cc(F)c3)c(=O)n21] | | 451.23 | 450.16 |
| 55 | 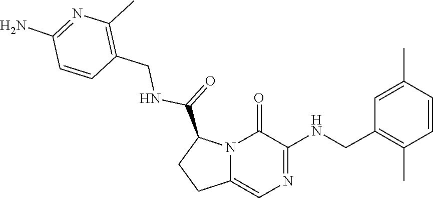 [SMILES: Cc1ccc(C)c(CNc2ncc3n(c2=O)[C@H](C(=O)NCc2ccc(N)nc2C)CC3)c1] | | 433.47 | 432.23 |
| 56 | 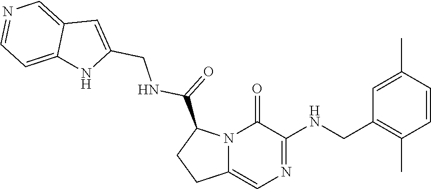 [SMILES: Cc1ccc(C)c(CNc2ncc3n(c2=O)[C@H](C(=O)NCc2cc4cnccc4[nH]2)CC3)c1] | | 443.44 | 442.21 |
| 57 | 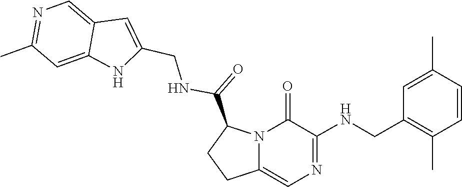 [SMILES: Cc1ccc(C)c(CNc2ncc3n(c2=O)[C@H](C(=O)NCc2cc4cnc(C)cc4[nH]2)CC3)c1] | | 457.44 | 456.23 |
| 58 | 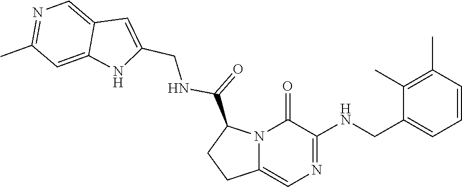 [SMILES: Cc1cc2[nH]c(CNC(=O)[C@@H]3CCc4cnc(NCc5cccc(C)c5C)c(=O)n43)cc2cn1] | | 457.53 | 456.23 |
| 59 | 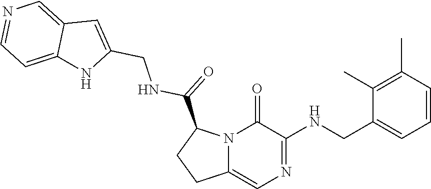 [SMILES: Cc1cccc(CNc2ncc3n(c2=O)[C@H](C(=O)NCc2cc4cnccc4[nH]2)CC3)c1C] | | 443.53 | 442.21 |
| 60 | 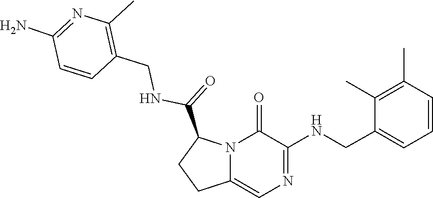 [SMILES: Cc1cccc(CNc2ncc3n(c2=O)[C@H](C(=O)NCc2ccc(N)nc2C)CC3)c1C] | | 433.47 | 432.23 |
| 61 | 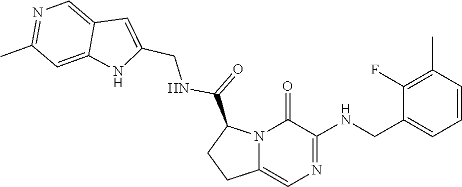 [SMILES: Cc1cc2[nH]c(CNC(=O)[C@@H]3CCc4cnc(NCc5cccc(C)c5F)c(=O)n43)cc2cn1] | | 461.47 | 460.20 |
| 62 | 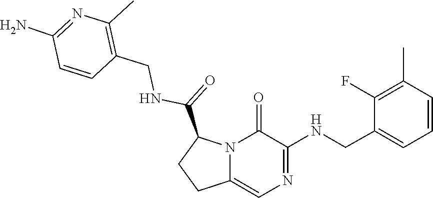 [SMILES: Cc1cccc(CNc2ncc3n(c2=O)[C@H](C(=O)NCc2ccc(N)nc2C)CC3)c1F] | | 437.49 | 436.20 |
| 63 | 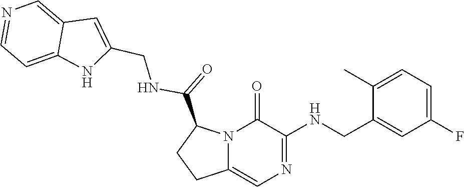 [SMILES: Cc1ccc(F)cc1CNc1ncc2n(c1=O)[C@H](C(=O)NCc1cc3cnccc3[nH]1)CC2] | | 447.47 | 446.19 |
| 64 | 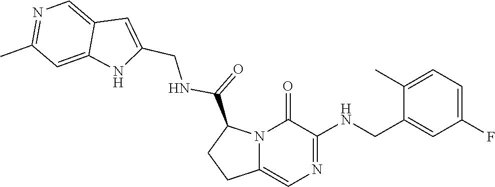 [SMILES: Cc1cc2[nH]c(CNC(=O)[C@@H]3CCc4cnc(NCc5cc(F)ccc5C)c(=O)n43)cc2cn1] | | 461.47 | 460.20 |
| 65 | 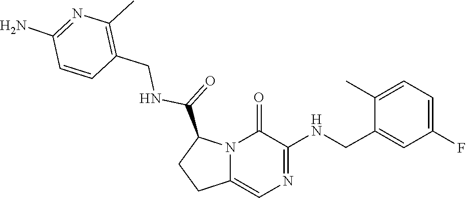 [SMILES: Cc1ccc(F)cc1CNc1ncc2n(c1=O)[C@H](C(=O)NCc1ccc(N)nc1C)CC2] | | 437.49 | 436.20 |
| 66 | 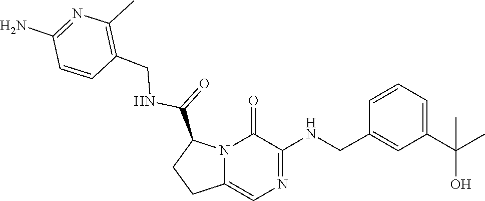 [SMILES: Cc1nc(N)ccc1CNC(=O)[C@@H]1CCc2cnc(NCc3cccc(C(C)(C)O)c3)c(=O)n21] | | 463.48 | 462.24 |
| 67 | 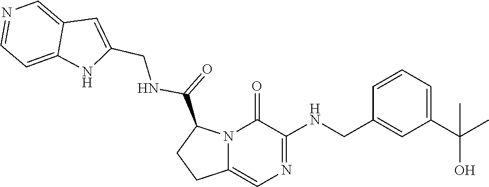 [SMILES: CC(C)(O)c1cccc(CNc2ncc3n(c2=O)[C@H](C(=O)NCc2cc4cnccc4[nH]2)CC3)c1] | | 473.54 | 472.22 |
| 68 | 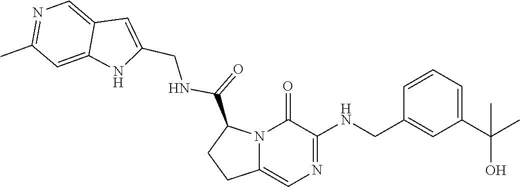 [SMILES: Cc1cc2[nH]c(CNC(=O)[C@@H]3CCc4cnc(NCc5cccc(C(C)(C)O)c5)c(=O)n43)cc2cn1] | | 487.54 | 486.24 |
| 69 | 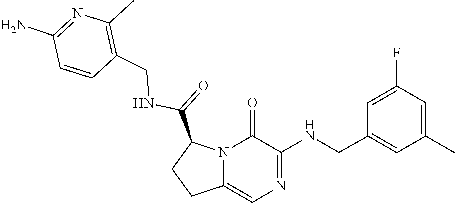 [SMILES: Cc1cc(F)cc(CNc2ncc3n(c2=O)[C@H](C(=O)NCc2ccc(N)nc2C)CC3)c1] | | 437.49 | 436.20 |
| 70 | 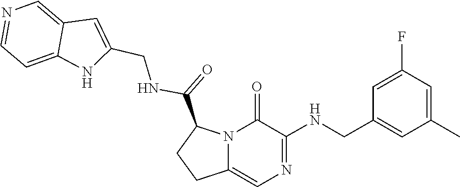 [SMILES: Cc1cc(F)cc(CNc2ncc3n(c2=O)[C@H](C(=O)NCc2cc4cnccc4[nH]2)CC3)c1] | | 447.47 | 446.19 |
| 71 | 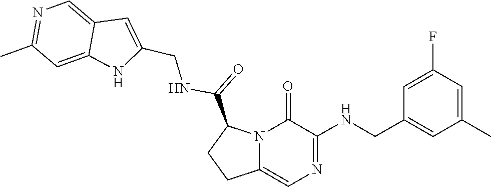 | | 461.47 | 460.20 |
| 72 | 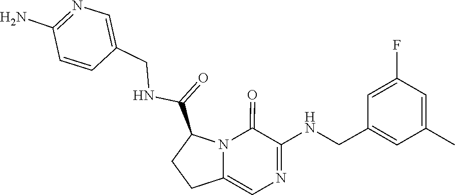 [SMILES: Cc1cc(F)cc(CNc2ncc3n(c2=O)[C@H](C(=O)NCc2ccc(N)nc2)CC3)c1] | | 423.50 | 422.19 |
| 73 | 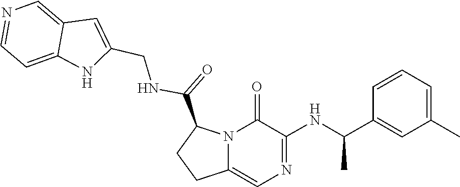 [SMILES: Cc1cccc([C@@H](C)Nc2ncc3n(c2=O)[C@H](C(=O)NCc2cc4cnccc4[nH]2)CC3)c1] | | 443.44 | 442.21 |
| 74 | 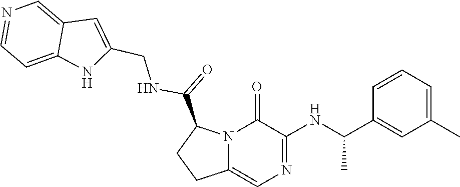 [SMILES: Cc1cccc([C@H](C)Nc2ncc3n(c2=O)[C@H](C(=O)NCc2cc4cnccc4[nH]2)CC3)c1] | | 443.44 | 442.21 |
| 75 | 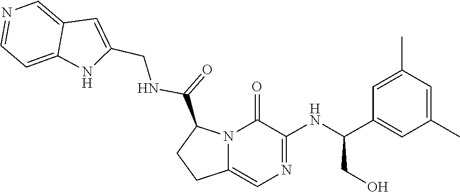 [SMILES: Cc1cc(C)cc([C@@H](CO)Nc2ncc3n(c2=O)[C@H](C(=O)NCc2cc4cnccc4[nH]2)CC3)c1] | | 473.45 | 472.22 |
| 76 | 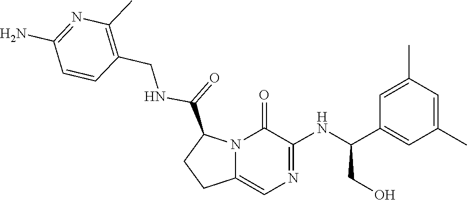 [SMILES: Cc1cc(C)cc([C@@H](CO)Nc2ncc3n(c2=O)[C@H](C(=O)NCc2ccc(N)nc2C)CC3)c1] | | 463.48 | 462.24 |
| 77 | 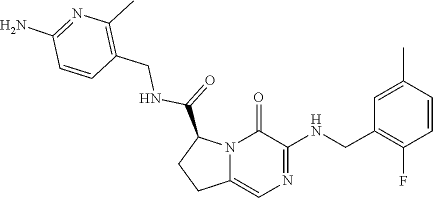 [SMILES: Cc1ccc(F)c(CNc2ncc3n(c2=O)[C@H](C(=O)NCc2ccc(N)nc2C)CC3)c1] | | 437.41 | 436.20 |
| 78 | 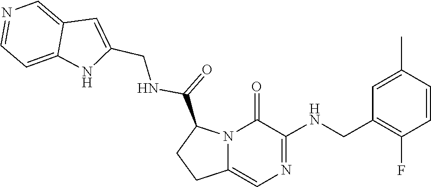 [SMILES: Cc1ccc(F)c(CNc2ncc3n(c2=O)[C@H](C(=O)NCc2cc4cnccc4[nH]2)CC3)c1] | | 447.38 | 446.19 |
| 79 | 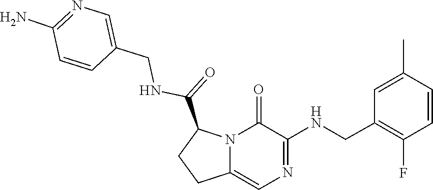 [SMILES: Cc1ccc(F)c(CNc2ncc3n(c2=O)[C@H](C(=O)NCc2ccc(N)nc2)CC3)c1] | | 423.41 | 422.19 |
| 80 | 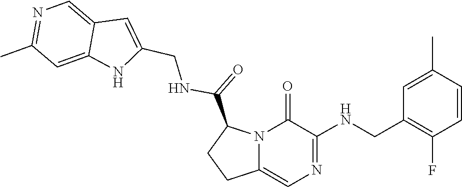 [SMILES: Cc1ccc(F)c(CNc2ncc3n(c2=O)[C@H](C(=O)NCc2cc4cnc(C)cc4[nH]2)CC3)c1] | | 461.47 | 460.20 |
| 81 | 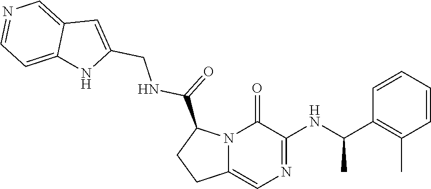 [SMILES: Cc1ccccc1[C@@H](C)Nc1ncc2n(c1=O)[C@H](C(=O)NCc1cc3cnccc3[nH]1)CC2] | | 443.44 | 442.21 |
| 82 | 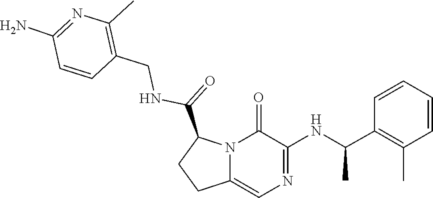 [SMILES: Cc1ccccc1[C@@H](C)Nc1ncc2n(c1=O)[C@H](C(=O)NCc1ccc(N)nc1C)CC2] | | 433.47 | 432.23 |
| 83 | 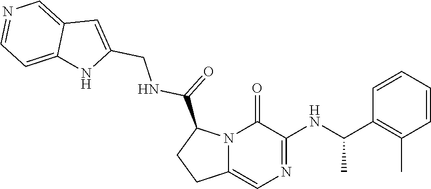 [SMILES: Cc1ccccc1[C@H](C)Nc1ncc2n(c1=O)[C@H](C(=O)NCc1cc3cnccc3[nH]1)CC2] | | 443.44 | 442.21 |
| 84 | 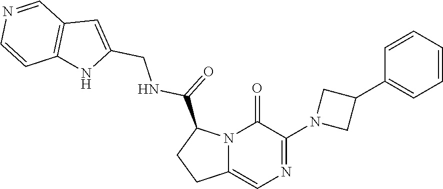 [SMILES: O=C(NCc1cc2cnccc2[nH]1)[C@@H]1CCc2cnc(N3CC(c4ccccc4)C3)c(=O)n21] | | 441.18 | 440.20 |
| 85 | 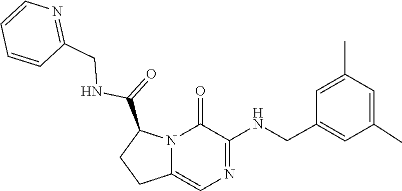 [SMILES: Cc1cc(C)cc(CNc2ncc3n(c2=O)[C@H](C(=O)NCc2ccccn2)CC3)c1] | | 404.17 | 403.20 |
| 86 | 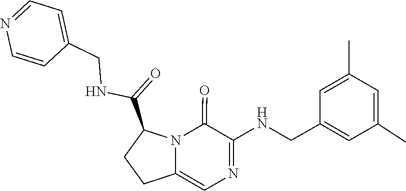 [SMILES: Cc1cc(C)cc(CNc2ncc3n(c2=O)[C@H](C(=O)NCc2ccncc2)CC3)c1] | | 404.15 | 403.20 |
| 87 | 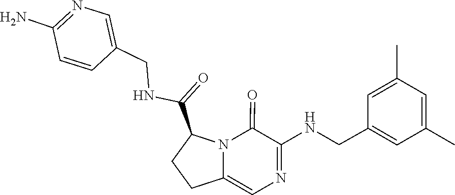 [SMILES: Cc1cc(C)cc(CNc2ncc3n(c2=O)[C@H](C(=O)NCc2ccc(N)nc2)CC3)c1] | | 418.88 | 418.21 |
| 88 | 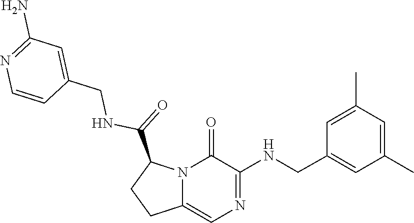 [SMILES: Cc1cc(C)cc(CNc2ncc3n(c2=O)[C@H](C(=O)NCc2ccnc(N)c2)CC3)c1] | | 419.19 | 418.21 |
| 89 | 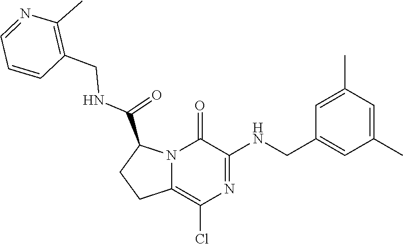 [SMILES: Cc1cc(C)cc(CNc2nc(Cl)c3n(c2=O)[C@H](C(=O)NCc2cccnc2C)CC3)c1] | TFA | 452.13 | 451.18 |
| 90 | 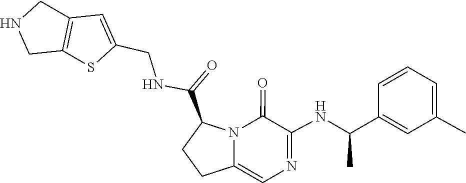 [SMILES: Cc1cccc([C@@H](C)Nc2ncc3n(c2=O)[C@H](C(=O)NCc2cc4c(s2)CNC4)CC3)c1] | | 450.44 | 449.19 |
| 91 | 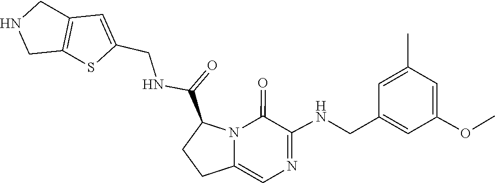 [SMILES: COc1cc(C)cc(CNc2ncc3n(c2=O)[C@H](C(=O)NCc2cc4c(s2)CNC4)CC3)c1] | | 466.45 | 465.18 |
| 92 | 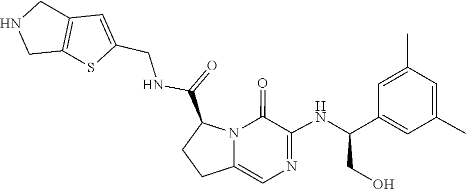 [SMILES: Cc1cc(C)cc([C@@H](CO)Nc2ncc3n(c2=O)[C@H](C(=O)NCc2cc4c(s2)CNC4)CC3)c1] | | 480.45 | 479.20 |
| 93 | 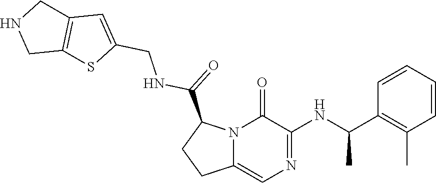 [SMILES: Cc1ccccc1[C@@H](C)Nc1ncc2n(c1=O)[C@H](C(=O)NCc1cc3c(s1)CNC3)CC2] | | 450.44 | 449.19 |
| 94 | 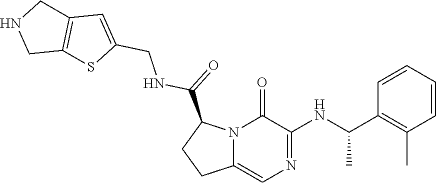 [SMILES: Cc1ccccc1[C@H](C)Nc1ncc2n(c1=O)[C@H](C(=O)NCc1cc3c(s1)CNC3)CC2] | | 450.44 | 449.19 |
| 95 | 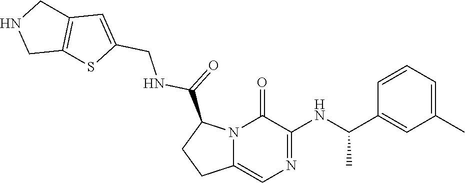 [SMILES: Cc1cccc([C@H](C)Nc2ncc3n(c2=O)[C@H](C(=O)NCc2cc4c(s2)CNC4)CC3)c1] | | 450.44 | 449.19 |
| 96 | 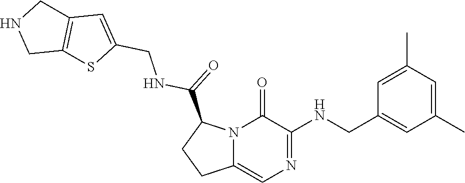 [SMILES: Cc1cc(C)cc(CNc2ncc3n(c2=O)[C@H](C(=O)NCc2cc4c(s2)CNC4)CC3)c1] | | 450.53 | 449.19 |
| 97 | 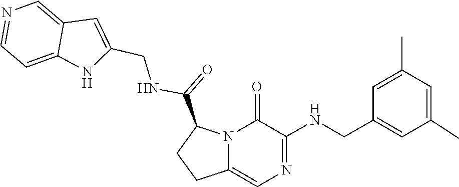 [SMILES: Cc1cc(C)cc(CNc2ncc3n(c2=O)[C@H](C(=O)NCc2cc4cnccc4[nH]2)CC3)c1] | TFA | 443.44 | 442.21 |
| 98 | 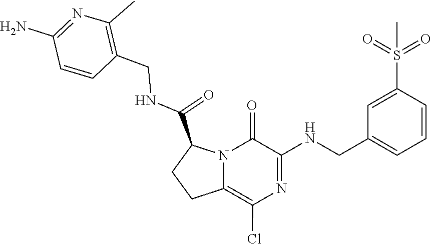 [SMILES: Cc1nc(N)ccc1CNC(=O)[C@@H]1CCc2c(Cl)nc(NCc3cccc(S(C)(=O)=O)c3)c(=O)n21] | TFA | 517.46 | 516.13 |
| 99 | 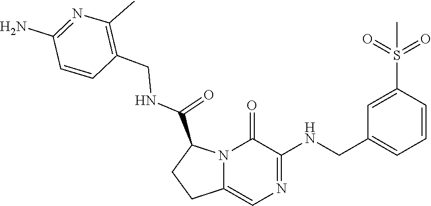 [SMILES: Cc1nc(N)ccc1CNC(=O)[C@@H]1CCc2cnc(NCc3cccc(S(C)(=O)=O)c3)c(=O)n21] | TFA | 483.51 | 482.17 |
| 100 | 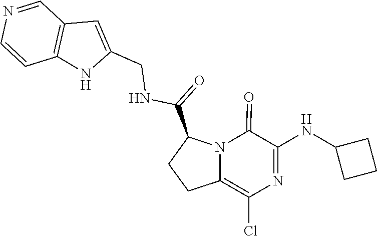 [SMILES: O=C(NCc1cc2cnccc2[nH]1)[C@@H]1CCc2c(Cl)nc(NC3CCC3)c(=O)n21] | TFA | 413.00 | 412.14 |
| 101 | 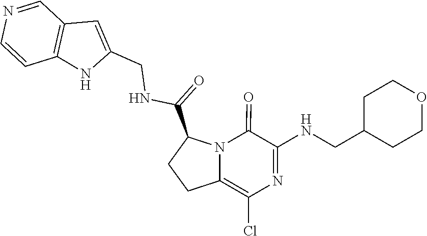 [SMILES: O=C(NCc1cc2cnccc2[nH]1)[C@@H]1CCc2c(Cl)nc(NCC3CCOCC3)c(=O)n21] | TFA | 457.02 | 456.17 |
| 102 | 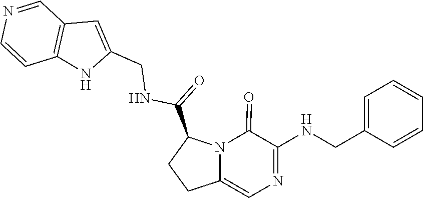 [SMILES: O=C(NCc1cc2cnccc2[nH]1)[C@@H]1CCc2cnc(NCc3ccccc3)c(=O)n21] | | 415.26 | 414.18 |
| 103 | 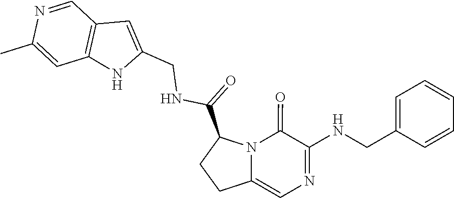 [SMILES: Cc1cc2[nH]c(CNC(=O)[C@@H]3CCc4cnc(NCc5ccccc5)c(=O)n43)cc2cn1] | | 429.18 | 428.20 |
| 104 | 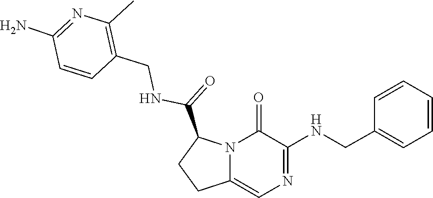 [SMILES: Cc1nc(N)ccc1CNC(=O)[C@@H]1CCc2cnc(NCc3ccccc3)c(=O)n21] | | 405.36 | 404.20 |
| 105 | 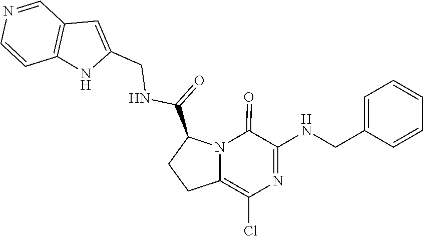 [SMILES: O=C(NCc1cc2cnccc2[nH]1)[C@@H]1CCc2c(Cl)nc(NCc3ccccc3)c(=O)n21] | | 449.06 | 448.14 |
| 106 | 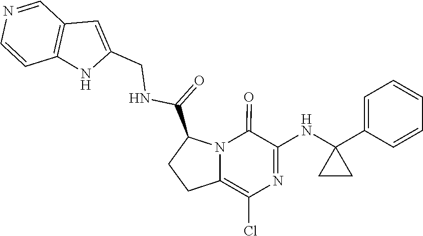 [SMILES: O=C(NCc1cc2cnccc2[nH]1)[C@@H]1CCc2c(Cl)nc(NC3(c4ccccc4)CC3)c(=O)n21] | | 475.15 | 474.16 |
| 107 | 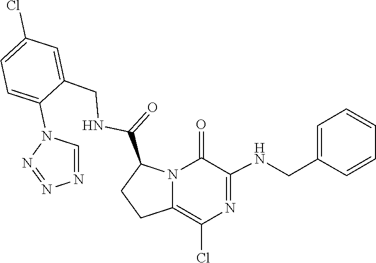 [SMILES: O=C(NCc1cc(Cl)ccc1-n1cnnn1)[C@@H]1CCc2c(Cl)nc(NCc3ccccc3)c(=O)n21] | | 510.91 | 510.11 |
| 108 | 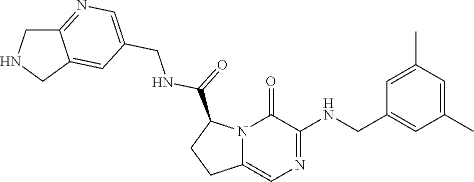 [SMILES: Cc1cc(C)cc(CNc2ncc3n(c2=O)[C@H](C(=O)NCc2cnc4c(c2)CNC4)CC3)c1] | | 445.21 | 444.23 |
| 109 | 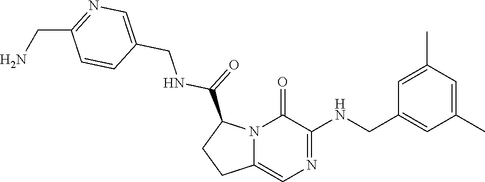 [SMILES: Cc1cc(C)cc(CNc2ncc3n(c2=O)[C@H](C(=O)NCc2ccc(CN)nc2)CC3)c1] | TFA | 433.11 | 432.23 |
| 110 | 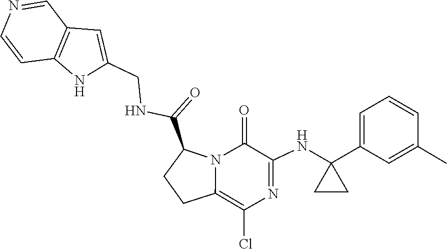 [SMILES: Cc1cccc(C2(Nc3nc(Cl)c4n(c3=O)[C@H](C(=O)NCc3cc5cnccc5[nH]3)CC4)CC2)c1] | TFA | 489.10 | 488.17 |
| 111 | 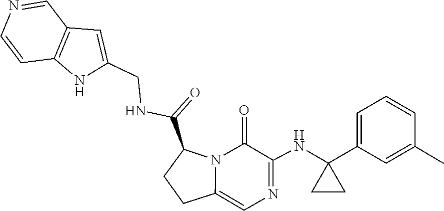 [SMILES: Cc1cccc(C2(Nc3ncc4n(c3=O)[C@H](C(=O)NCc3cc5cnccc5[nH]3)CC4)CC2)c1] | TFA | 455.17 | 454.21 |
| 112 | 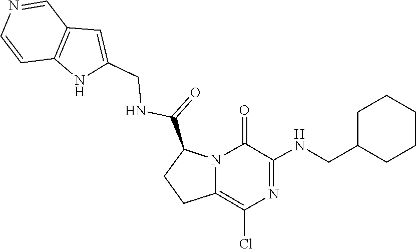 [SMILES: O=C(NCc1cc2cnccc2[nH]1)[C@@H]1CCc2c(Cl)nc(NCC3CCCCC3)c(=O)n21] | TFA | 455.37 | 454.19 |
| 113 | 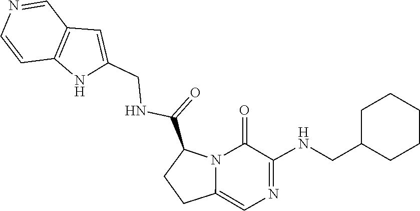 [SMILES: O=C(NCc1cc2cnccc2[nH]1)[C@@H]1CCc2cnc(NCC3CCCCC3)c(=O)n21] | TFA | 421.40 | 420.23 |
| 114 | 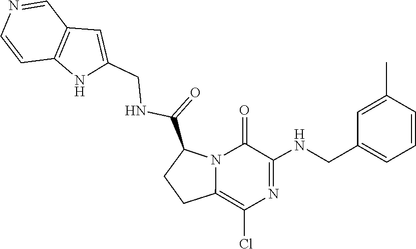 [SMILES: Cc1cccc(CNc2nc(Cl)c3n(c2=O)[C@H](C(=O)NCc2cc4cnccc4[nH]2)CC3)c1] | TFA | 463.33 | 462.16 |
| 115 | 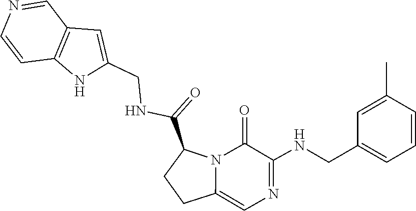 [SMILES: Cc1cccc(CNc2ncc3n(c2=O)[C@H](C(=O)NCc2cc4cnccc4[nH]2)CC3)c1] | TFA | 429.38 | 428.20 |
| 116 | 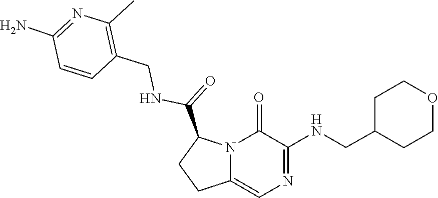 [SMILES: Cc1nc(N)ccc1CNC(=O)[C@@H]1CCc2cnc(NCC3CCOCC3)c(=O)n21] | | 413.41 | 412.22 |
| 117 | 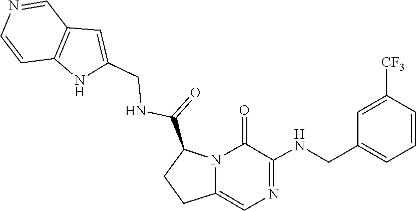 [SMILES: O=C(NCc1cc2cnccc2[nH]1)[C@@H]1CCc2cnc(NCc3cccc(C(F)(F)F)c3)c(=O)n21] | TFA | 483.36 | 482.17 |
| 118 | 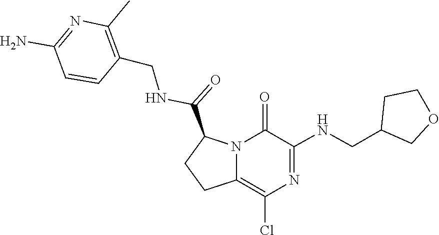 [SMILES: Cc1nc(N)ccc1CNC(=O)[C@@H]1CCc2c(Cl)nc(NCC3CCOC3)c(=O)n21] | | 433.19 | 432.17 |
| 119 | 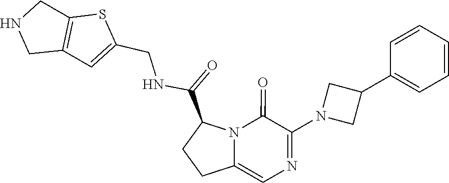 [SMILES: O=C(NCc1cc2c(s1)CNC2)[C@@H]1CCc2cnc(N3CC(c4ccccc4)C3)c(=O)n21] | | 448.20 | 447.17 |
| 120 | 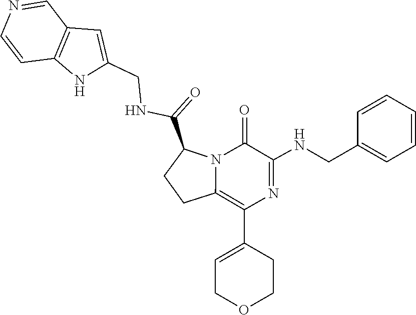 [SMILES: O=C(NCc1cc2cnccc2[nH]1)[C@@H]1CCc2c(C3=CCOCC3)nc(NCc3ccccc3)c(=O)n21] | | 497.18 | 496.22 |
| 121 | 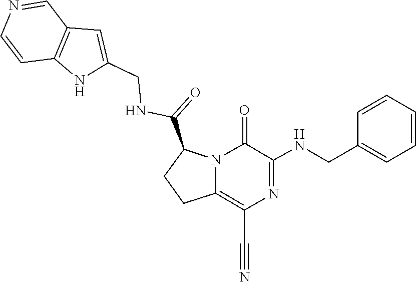 [SMILES: N#Cc1nc(NCc2ccccc2)c(=O)n2c1CC[C@H]2C(=O)NCc1cc2cnccc2[nH]1] | TFA | 440.19 | 439.18 |
| 122 | 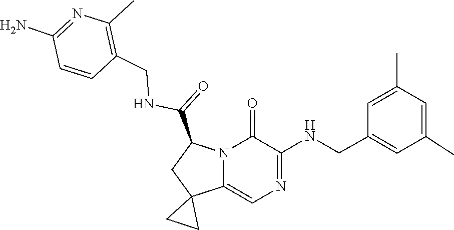 [SMILES: Cc1cc(C)cc(CNc2ncc3n(c2=O)[C@H](C(=O)NCc2ccc(N)nc2C)CC32CC2)c1] | TFA | 459.17 | 458.24 |
| 123 | 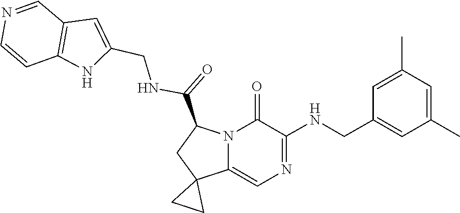 [SMILES: Cc1cc(C)cc(CNc2ncc3n(c2=O)[C@H](C(=O)NCc2cc4cnccc4[nH]2)CC32CC2)c1] | TFA | 469.20 | 468.23 |
| 124 | 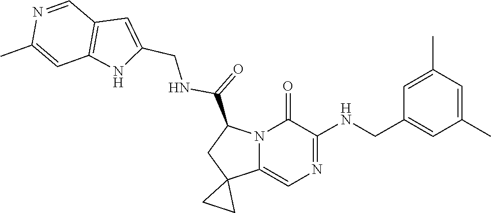 | TFA | 483.22 | 482.24 |
| 125 | 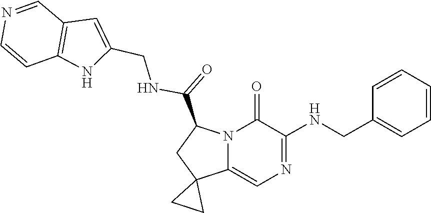 [SMILES: O=C(NCc1cc2cnccc2[nH]1)[C@@H]1CC2(CC2)c2cnc(NCc3ccccc3)c(=O)n21] | | 441.37 | 440.20 |
| 126 | 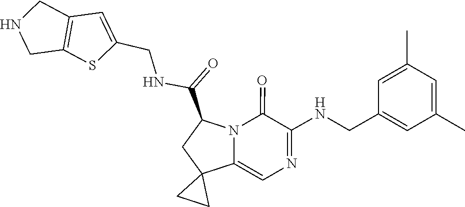 [SMILES: Cc1cc(C)cc(CNc2ncc3n(c2=O)[C@H](C(=O)NCc2cc4c(s2)CNC4)CC32CC2)c1] | | 476.18 | 475.20 |
| 127 | 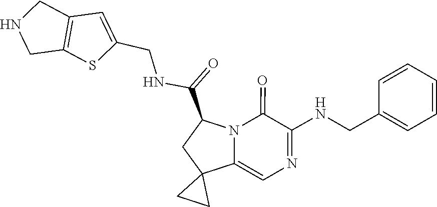 [SMILES: O=C(NCc1cc2c(s1)CNC2)[C@@H]1CC2(CC2)c2cnc(NCc3ccccc3)c(=O)n21] | TFA | 448.37 | 447.17 |
| 128 | 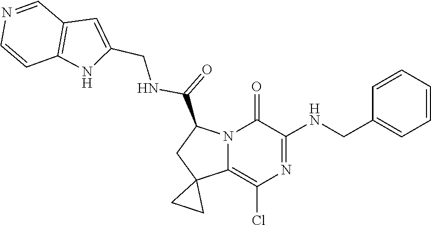 [SMILES: O=C(NCc1cc2cnccc2[nH]1)[C@@H]1CC2(CC2)c2c(Cl)nc(NCc3ccccc3)c(=O)n21] | | 475.32 | 474.16 |
| 129 | 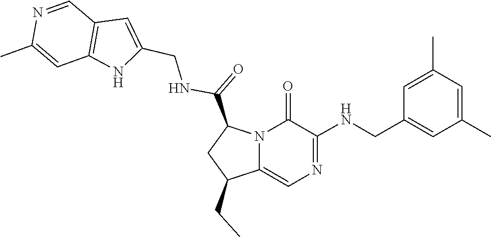 | | 483.27 (ES−; M − H) | 484.26 |
| 130 | 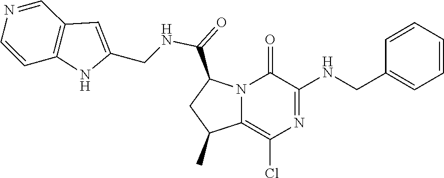 [SMILES: C[C@H]1C[C@@H](C(=O)NCc2cc3cnccc3[nH]2)n2c1c(Cl)nc(NCc1ccccc1)c2=O] | | 463.17 | 462.16 |
| 131 | 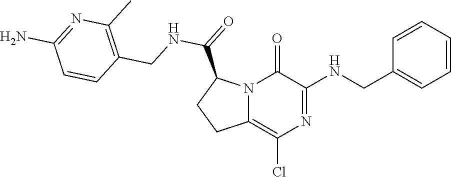 [SMILES: Cc1nc(N)ccc1CNC(=O)[C@@H]1CCc2c(Cl)nc(NCc3ccccc3)c(=O)n21] | | 439.16 | 438.16 |
| 132 | 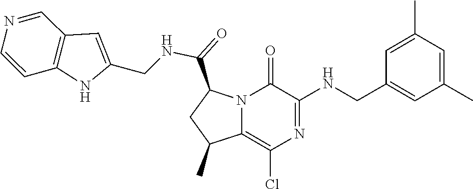 [SMILES: Cc1cc(C)cc(CNc2nc(Cl)c3n(c2=O)[C@H](C(=O)NCc2cc4cnccc4[nH]2)C[C@@H]3C)c1] | | 491.17 | 490.19 |
| 133 | 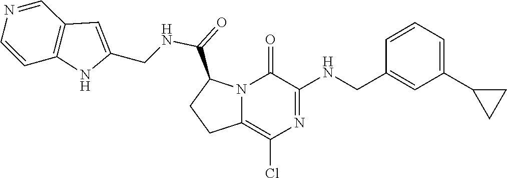 [SMILES: O=C(NCc1cc2cnccc2[nH]1)[C@@H]1CCc2c(Cl)nc(NCc3cccc(C4CC4)c3)c(=O)n21] | | 489.17 | 488.17 |
| 134 | 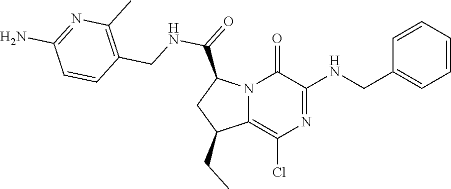 [SMILES: CC[C@H]1C[C@@H](C(=O)NCc2ccc(N)nc2C)n2c1c(Cl)nc(NCc1ccccc1)c2=O] | | 467.42 | 466.19 |
| 135 | 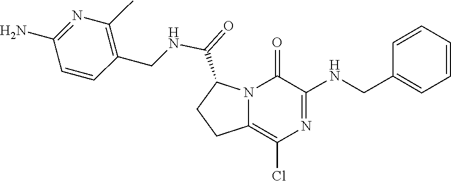 [SMILES: Cc1nc(N)ccc1CNC(=O)[C@H]1CCc2c(Cl)nc(NCc3ccccc3)c(=O)n21] | | 439.42 | 438.16 |
| 136 | 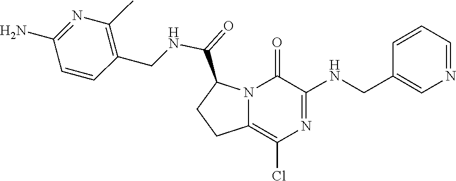 [SMILES: Cc1nc(N)ccc1CNC(=O)[C@@H]1CCc2c(Cl)nc(NCc3cccnc3)c(=O)n21] | | 440.42 | 439.15 |
| 137 | 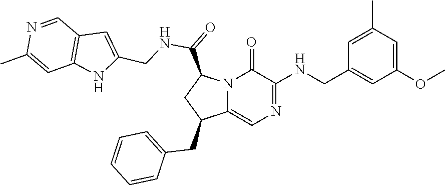 | | 563.56 | 562.27 |
| 138 | 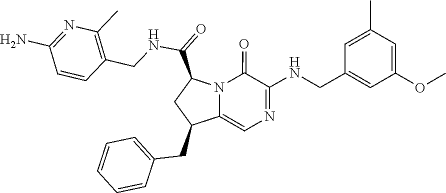 [SMILES: COc1cc(C)cc(CNc2ncc3n(c2=O)[C@H](C(=O)NCc2ccc(N)nc2C)C[C@@H]3Cc2ccccc2)c1] | | 539.50 | 538.27 |
| 139 | 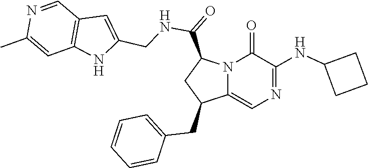 [SMILES: Cc1cc2[nH]c(CNC(=O)[C@@H]3C[C@H](Cc4ccccc4)c4cnc(NC5CCC5)c(=O)n43)cc2cn1] | | 483.51 | 482.24 |
| 140 | 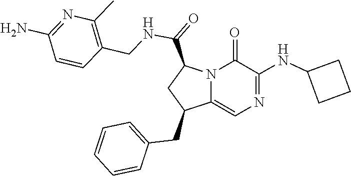 [SMILES: Cc1nc(N)ccc1CNC(=O)[C@@H]1C[C@H](Cc2ccccc2)c2cnc(NC3CCC3)c(=O)n21] | | 459.45 | 458.24 |
| 141 | 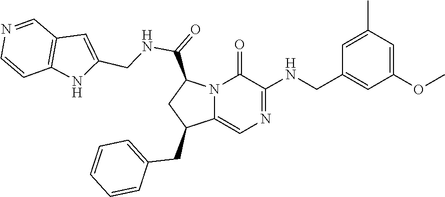 [SMILES: COc1cc(C)cc(CNc2ncc3n(c2=O)[C@H](C(=O)NCc2cc4cnccc4[nH]2)C[C@@H]3Cc2ccccc2)c1] | | 549.56 | 548.25 |
| 142 | 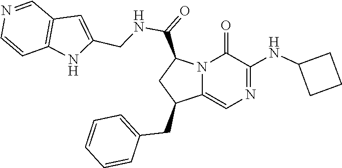 [SMILES: O=C(NCc1cc2cnccc2[nH]1)[C@@H]1C[C@H](Cc2ccccc2)c2cnc(NC3CCC3)c(=O)n21] | | 469.52 | 468.23 |
| 143 | 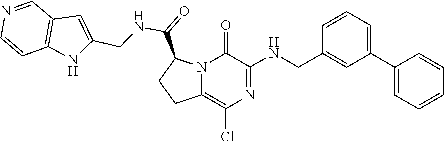 [SMILES: O=C(NCc1cc2cnccc2[nH]1)[C@@H]1CCc2c(Cl)nc(NCc3cccc(-c4ccccc4)c3)c(=O)n21] | | 525.19 | 524.17 |
| 144 | 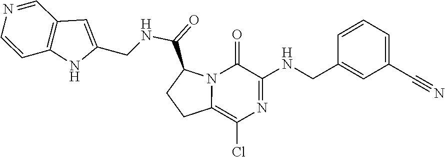 [SMILES: N#Cc1cccc(CNc2nc(Cl)c3n(c2=O)[C@H](C(=O)NCc2cc4cnccc4[nH]2)CC3)c1] | | 474.15 | 473.14 |
| 145 | 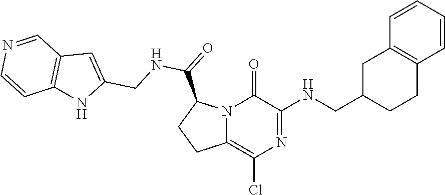 [SMILES: O=C(NCc1cc2cnccc2[nH]1)[C@@H]1CCc2c(Cl)nc(NCC3CCc4ccccc4C3)c(=O)n21] | | 503.20 | 502.19 |
| 146 | 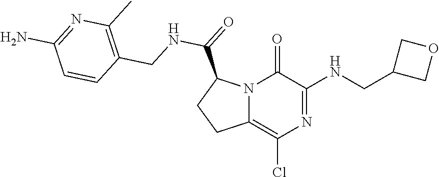 [SMILES: Cc1nc(N)ccc1CNC(=O)[C@@H]1CCc2c(Cl)nc(NCC3COC3)c(=O)n21] | | 419.17 | 418.15 |
| 147 | 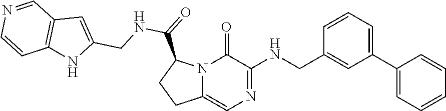 [SMILES: O=C(NCc1cc2cnccc2[nH]1)[C@@H]1CCc2cnc(NCc3cccc(-c4ccccc4)c3)c(=O)n21] | | 491.21 | 490.21 |
| 148 | 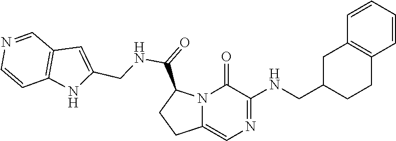 [SMILES: O=C(NCc1cc2cnccc2[nH]1)[C@@H]1CCc2cnc(NCC3CCc4ccccc4C3)c(=O)n21] | | 469.23 | 468.23 |
| 149 | 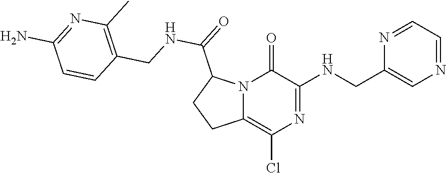 [SMILES: Cc1nc(N)ccc1CNC(=O)C1CCc2c(Cl)nc(NCc3cnccn3)c(=O)n21] | | 441.43 | 440.15 |
| 150 | 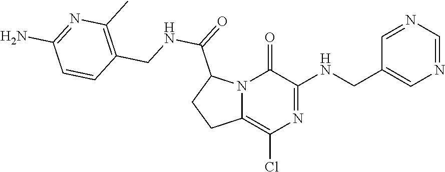 [SMILES: Cc1nc(N)ccc1CNC(=O)C1CCc2c(Cl)nc(NCc3cncnc3)c(=O)n21] | | 441.43 | 440.15 |
| 151 | 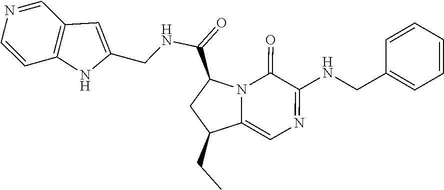 [SMILES: CC[C@H]1C[C@@H](C(=O)NCc2cc3cnccc3[nH]2)n2c1cnc(NCc1ccccc1)c2=O] | | 442.21 | 441.43 |
| 152 | 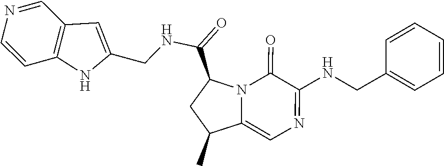 [SMILES: C[C@H]1C[C@@H](C(=O)NCc2cc3cnccc3[nH]2)n2c1cnc(NCc1ccccc1)c2=O] | | 429.21 | 428.20 |
| 153 | 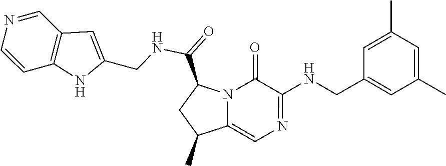 [SMILES: Cc1cc(C)cc(CNc2ncc3n(c2=O)[C@H](C(=O)NCc2cc4cnccc4[nH]2)C[C@@H]3C)c1] | | 457.23 | 456.23 |
| 154 | 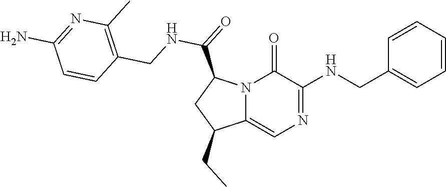 [SMILES: CC[C@H]1C[C@@H](C(=O)NCc2ccc(N)nc2C)n2c1cnc(NCc1ccccc1)c2=O] | | 433.47 | 432.23 |
| 155 | 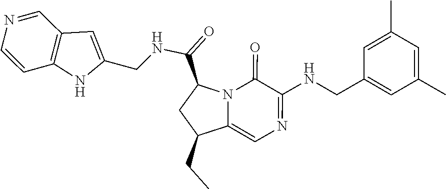 [SMILES: CC[C@H]1C[C@@H](C(=O)NCc2cc3cnccc3[nH]2)n2c1cnc(NCc1cc(C)cc(C)c1)c2=O] | | 471.53 | 470.24 |
| 156 | 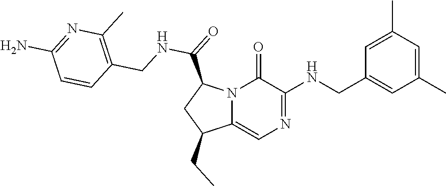 [SMILES: CC[C@H]1C[C@@H](C(=O)NCc2ccc(N)nc2C)n2c1cnc(NCc1cc(C)cc(C)c1)c2=O] | | 461.55 | 460.26 |
| 157 | 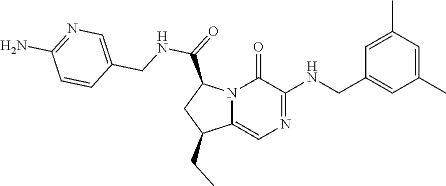 [SMILES: CC[C@H]1C[C@@H](C(=O)NCc2ccc(N)nc2)n2c1cnc(NCc1cc(C)cc(C)c1)c2=O] | | 447.47 | 446.24 |
| 158 | 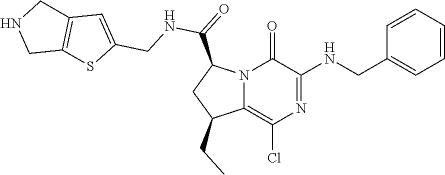 [SMILES: CC[C@H]1C[C@@H](C(=O)NCc2cc3c(s2)CNC3)n2c1c(Cl)nc(NCc1ccccc1)c2=O] | HCl | 484.39 | 483.15 |
| 159 | 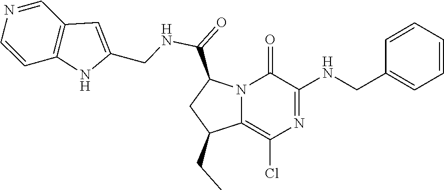 [SMILES: CC[C@H]1C[C@@H](C(=O)NCc2cc3cnccc3[nH]2)n2c1c(Cl)nc(NCc1ccccc1)c2=O] | | 477.16 | 476.17 |
| 160 | 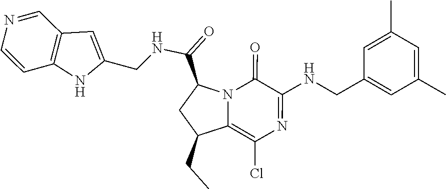 [SMILES: CC[C@H]1C[C@@H](C(=O)NCc2cc3cnccc3[nH]2)n2c1c(Cl)nc(NCc1cc(C)cc(C)c1)c2=O] | | 505.18 | 504.20 |
| 161 | 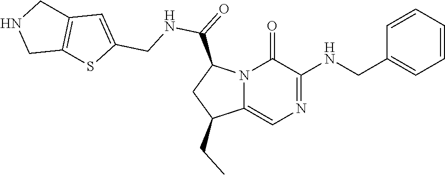 [SMILES: CC[C@H]1C[C@@H](C(=O)NCc2cc3c(s2)CNC3)n2c1cnc(NCc1ccccc1)c2=O] | HCl | 450.44 | 449.19 |
| 162 | 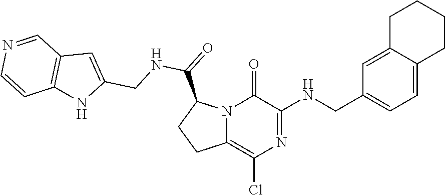 [SMILES: O=C(NCc1cc2cnccc2[nH]1)[C@@H]1CCc2c(Cl)nc(NCc3ccc4c(c3)CCCC4)c(=O)n21] | | 503.16 | 502.19 |
| 163 | 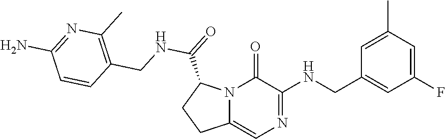 [SMILES: Cc1cc(F)cc(CNc2ncc3n(c2=O)[C@@H](C(=O)NCc2ccc(N)nc2C)CC3)c1] | | 437.41 | 436.20 |
| 164 | 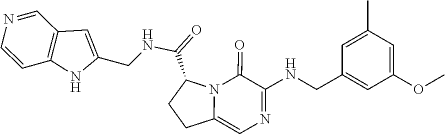 [SMILES: COc1cc(C)cc(CNc2ncc3n(c2=O)[C@@H](C(=O)NCc2cc4cnccc4[nH]2)CC3)c1] | | 458.21 (ES−; M − H) | 459.45 |
| 165 | 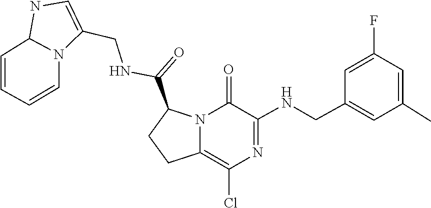 [SMILES: Cc1cc(F)cc(CNc2nc(Cl)c3n(c2=O)[C@H](C(=O)NCC2=CNC4C=CC=CN24)CC3)c1] | TFA | 480.15 | 479.15 |
| 166 | 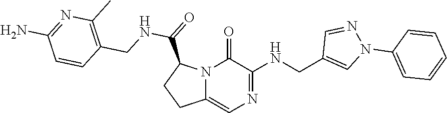 [SMILES: Cc1nc(N)ccc1CNC(=O)[C@@H]1CCc2cnc(NCc3cnn(-c4ccccc4)c3)c(=O)n21] | TFA | 471.55 | 470.22 |
| 167 | 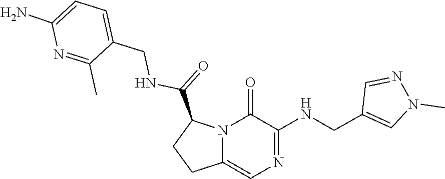 [SMILES: Cc1nc(N)ccc1CNC(=O)[C@@H]1CCc2cnc(NCc3cnn(C)c3)c(=O)n21] | TFA | 409.22 | 408.20 |
| 168 | 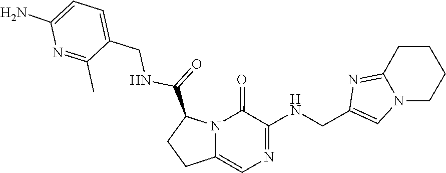 [SMILES: Cc1nc(N)ccc1CNC(=O)[C@@H]1CCc2cnc(NCc3cn4c(n3)CCCC4)c(=O)n21] | TFA | 449.23 | 448.23 |
| 169 | 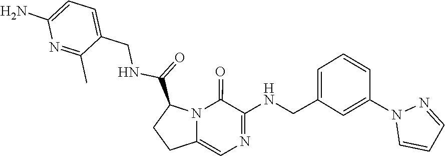 [SMILES: Cc1nc(N)ccc1CNC(=O)[C@@H]1CCc2cnc(NCc3cccc(-n4cccn4)c3)c(=O)n21] | TFA | 471.21 | 470.22 |
| 170 | 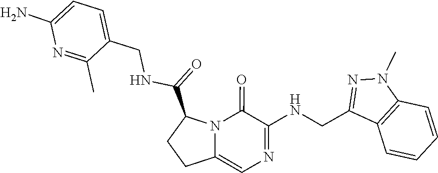 [SMILES: Cc1nc(N)ccc1CNC(=O)[C@@H]1CCc2cnc(NCc3nn(C)c4ccccc34)c(=O)n21] | TFA | 459.26 | 458.22 |
| 171 | 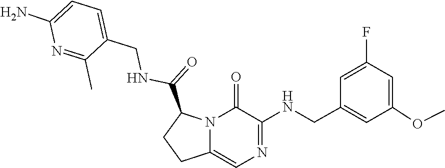 [SMILES: COc1cc(F)cc(CNc2ncc3n(c2=O)[C@H](C(=O)NCc2ccc(N)nc2C)CC3)c1] | TFA | 453.21 | 452.20 |
| 172 | 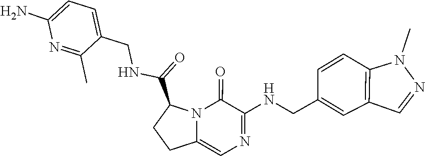 [SMILES: Cc1nc(N)ccc1CNC(=O)[C@@H]1CCc2cnc(NCc3ccc4c(cnn4C)c3)c(=O)n21] | | 459.26 | 458.22 |
| 173 | 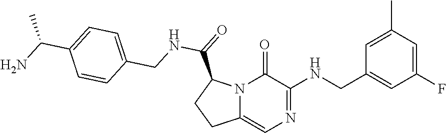 [SMILES: Cc1cc(F)cc(CNc2ncc3n(c2=O)[C@H](C(=O)NCc2ccc([C@@H](C)N)cc2)CC3)c1] | TFA | 450.20 | 449.22 |
| 174 | 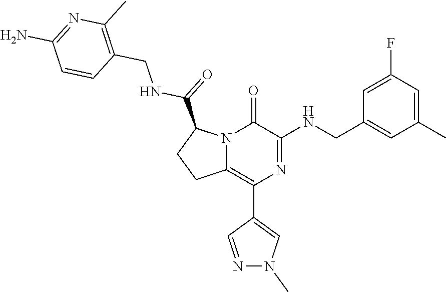 [SMILES: Cc1cc(F)cc(CNc2nc(-c3cnn(C)c3)c3n(c2=O)[C@H](C(=O)NCc2ccc(N)nc2C)CC3)c1] | TFA | 517.26 | 516.24 |
| 175 | 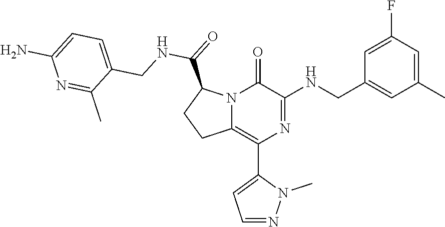 [SMILES: Cc1cc(F)cc(CNc2nc(-c3ccnn3C)c3n(c2=O)[C@H](C(=O)NCc2ccc(N)nc2C)CC3)c1] | TFA | 517.62 | 516.24 |
| 176 | 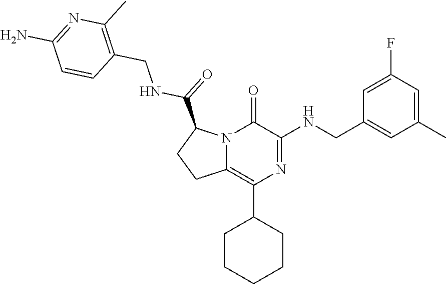 [SMILES: Cc1cc(F)cc(CNc2nc(C3CCCCC3)c3n(c2=O)[C@H](C(=O)NCc2ccc(N)nc2C)CC3)c1] | TFA | 519.26 | 518.28 |
| 177 | 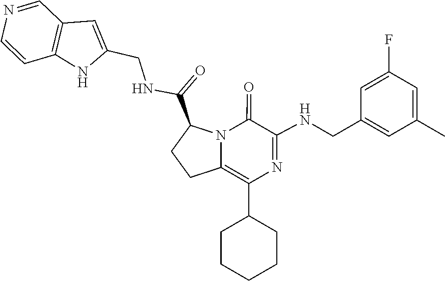 [SMILES: Cc1cc(F)cc(CNc2nc(C3CCCCC3)c3n(c2=O)[C@H](C(=O)NCc2cc4cnccc4[nH]2)CC3)c1] | TFA | 529.26 | 528.26 |
| 178 | 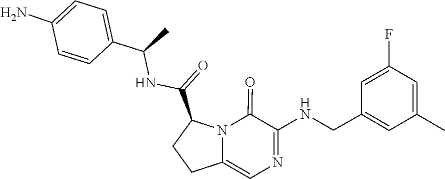 [SMILES: Cc1cc(F)cc(CNc2ncc3n(c2=O)[C@H](C(=O)N[C@H](C)c2ccc(N)cc2)CC3)c1] | TFA | 450.24 | 449.22 |
| 179 | 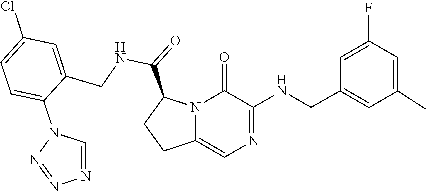 [SMILES: Cc1cc(F)cc(CNc2ncc3n(c2=O)[C@H](C(=O)NCc2cc(Cl)ccc2-n2cnnn2)CC3)c1] | | 509.53 | 508.15 |
| 180 | 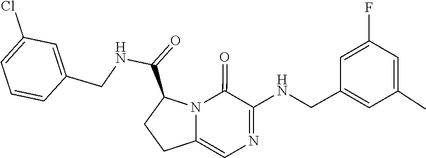 [SMILES: Cc1cc(F)cc(CNc2ncc3n(c2=O)[C@H](C(=O)NCc2cccc(Cl)c2)CC3)c1] | | 441.56 | 440.14 |
| 181 | 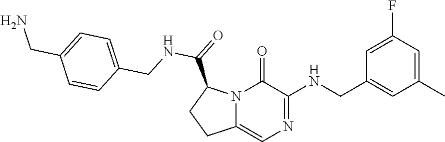 [SMILES: Cc1cc(F)cc(CNc2ncc3n(c2=O)[C@H](C(=O)NCc2ccc(CN)cc2)CC3)c1] | TFA | 436.66 | 435.21 |
| 182 | 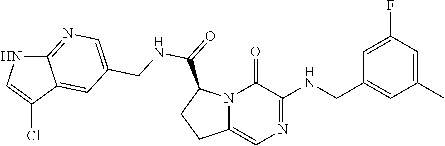 [SMILES: Cc1cc(F)cc(CNc2ncc3n(c2=O)[C@H](C(=O)NCc2cnc4[nH]cc(Cl)c4c2)CC3)c1] | | 481.54 | 480.15 |
| 183 | 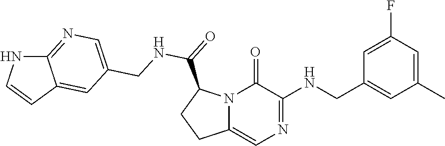 [SMILES: Cc1cc(F)cc(CNc2ncc3n(c2=O)[C@H](C(=O)NCc2cnc4[nH]ccc4c2)CC3)c1] | | 447.56 | 446.19 |
| 184 | 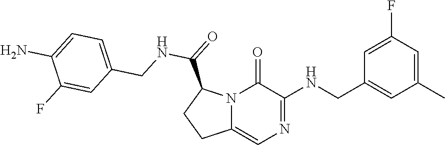 [SMILES: Cc1cc(F)cc(CNc2ncc3n(c2=O)[C@H](C(=O)NCc2ccc(N)c(F)c2)CC3)c1] | | 440.56 | 439.18 |
| 185 | 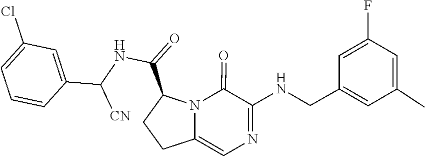 [SMILES: Cc1cc(F)cc(CNc2ncc3n(c2=O)[C@H](C(=O)NC(C#N)c2cccc(Cl)c2)CC3)c1] | | 466.55 | 465.14 |
| 186 | 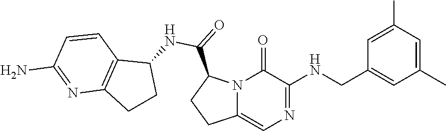 [SMILES: Cc1cc(C)cc(CNc2ncc3n(c2=O)[C@H](C(=O)N[C@@H]2CCc4nc(N)ccc42)CC3)c1] | | 445.56 | 444.23 |
| 187 | 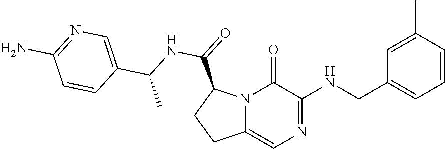 [SMILES: Cc1cccc(CNc2ncc3n(c2=O)[C@H](C(=O)N[C@H](C)c2ccc(N)nc2)CC3)c1] | | 419.38 | 418.21 |
| 188 | 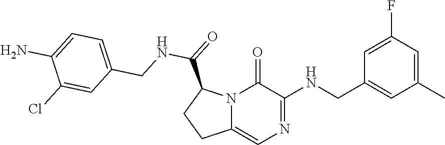 [SMILES: Cc1cc(F)cc(CNc2ncc3n(c2=O)[C@H](C(=O)NCc2ccc(N)c(Cl)c2)CC3)c1] | | 456.55 | 455.15 |
| 189 | 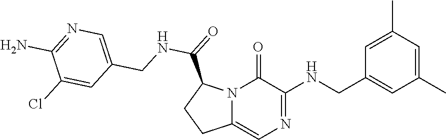 [SMILES: Cc1cc(C)cc(CNc2ncc3n(c2=O)[C@H](C(=O)NCc2cnc(N)c(Cl)c2)CC3)c1] | | 453.56 | 452.17 |
| 190 | 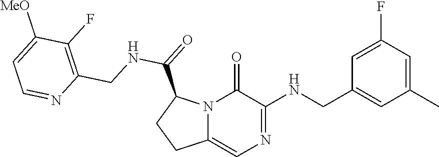 [SMILES: COc1ccnc(CNC(=O)[C@@H]2CCc3cnc(NCc4cc(C)cc(F)c4)c(=O)n32)c1F] | TFA | 456.55 | 455.18 |
| 191 | 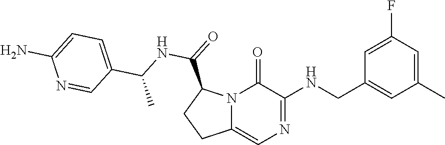 [SMILES: Cc1cc(F)cc(CNc2ncc3n(c2=O)[C@H](C(=O)N[C@H](C)c2ccc(N)nc2)CC3)c1] | TFA | 437.56 | 436.20 |
| 192 | 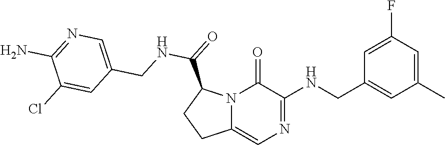 [SMILES: Cc1cc(F)cc(CNc2ncc3n(c2=O)[C@H](C(=O)NCc2cnc(N)c(Cl)c2)CC3)c1] | TFA | 457.55 | 456.15 |
| 193 | 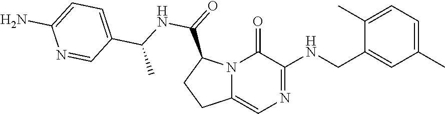 [SMILES: Cc1ccc(C)c(CNc2ncc3n(c2=O)[C@H](C(=O)N[C@H](C)c2ccc(N)nc2)CC3)c1] | TFA | 433.56 | 432.23 |
| 194 | 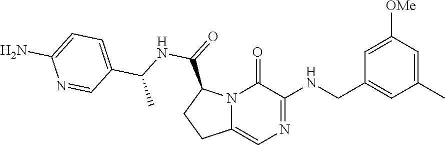 [SMILES: COc1cc(C)cc(CNc2ncc3n(c2=O)[C@H](C(=O)N[C@H](C)c2ccc(N)nc2)CC3)c1] | TFA | 449.23 | 448.22 |
| 195 | 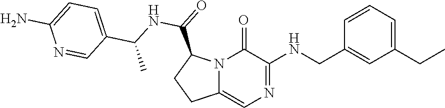 [SMILES: CCc1cccc(CNc2ncc3n(c2=O)[C@H](C(=O)N[C@H](C)c2ccc(N)nc2)CC3)c1] | TFA | 433.56 | 432.23 |
| 196 | 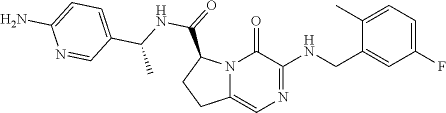 [SMILES: Cc1ccc(F)cc1CNc1ncc2n(c1=O)[C@H](C(=O)N[C@H](C)c1ccc(N)nc1)CC2] | TFA | 437.56 | 436.20 |
| 197 | 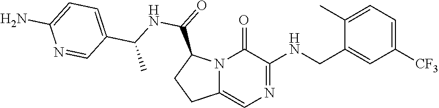 [SMILES: Cc1ccc(C(F)(F)F)cc1CNc1ncc2n(c1=O)[C@H](C(=O)N[C@H](C)c1ccc(N)nc1)CC2] | TFA | 487.54 | 486.20 |
| 198 | 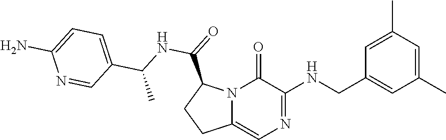 [SMILES: Cc1cc(C)cc(CNc2ncc3n(c2=O)[C@H](C(=O)N[C@H](C)c2ccc(N)nc2)CC3)c1] | TFA | 433.56 | 432.23 |
| 199 | 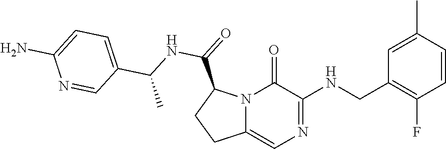 [SMILES: Cc1ccc(F)c(CNc2ncc3n(c2=O)[C@H](C(=O)N[C@H](C)c2ccc(N)nc2)CC3)c1] | TFA | 437.56 | 436.20 |
| 200 | 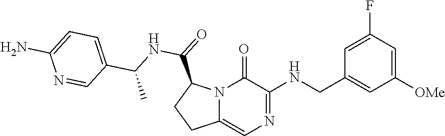 [SMILES: COc1cc(F)cc(CNc2ncc3n(c2=O)[C@H](C(=O)N[C@H](C)c2ccc(N)nc2)CC3)c1] | TFA | 453.46 | 452.20 |
| 201 | 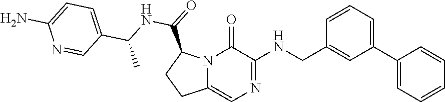 [SMILES: C[C@@H](NC(=O)[C@@H]1CCc2cnc(NCc3cccc(-c4ccccc4)c3)c(=O)n21)c1ccc(N)nc1] | | 481.54 | 480.23 |
| 202 | 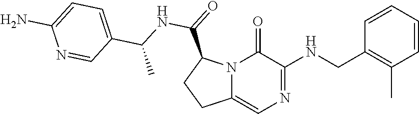 [SMILES: Cc1ccccc1CNc1ncc2n(c1=O)[C@H](C(=O)N[C@H](C)c1ccc(N)nc1)CC2] | | 419.57 | 418.21 |
| 203 | 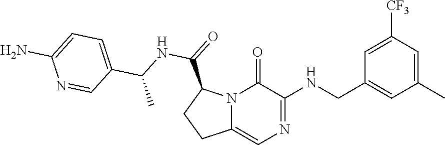 [SMILES: Cc1cc(CNc2ncc3n(c2=O)[C@H](C(=O)N[C@H](C)c2ccc(N)nc2)CC3)cc(C(F)(F)F)c1] | | 487.54 | 486.20 |
| 204 | 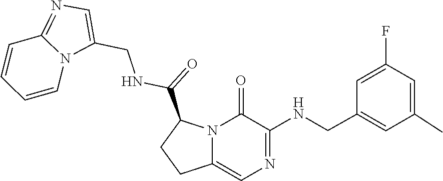 [SMILES: Cc1cc(F)cc(CNc2ncc3n(c2=O)[C@H](C(=O)NCc2cnc4ccccn24)CC3)c1] | TFA | 447.17 | 446.19 |
| 205 | 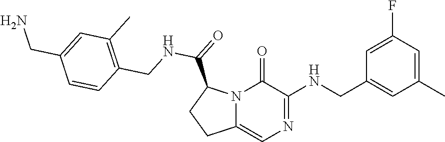 [SMILES: Cc1cc(F)cc(CNc2ncc3n(c2=O)[C@H](C(=O)NCc2ccc(CN)cc2C)CC3)c1] | TFA | 450.66 | 449.22 |
| 206 | 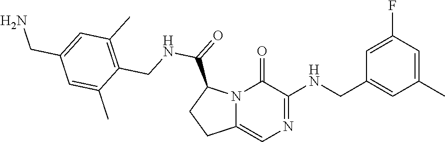 [SMILES: Cc1cc(F)cc(CNc2ncc3n(c2=O)[C@H](C(=O)NCc2c(C)cc(CN)cc2C)CC3)c1] | TFA | 464.65 | 463.24 |
| 207 | 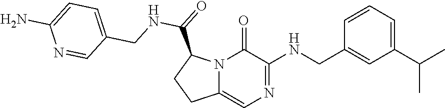 [SMILES: CC(C)c1cccc(CNc2ncc3n(c2=O)[C@H](C(=O)NCc2ccc(N)nc2)CC3)c1] | TFA | 433.66 | 432.23 |
| 208 | 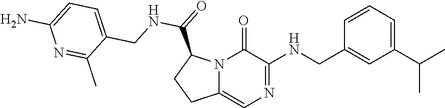 [SMILES: Cc1nc(N)ccc1CNC(=O)[C@@H]1CCc2cnc(NCc3cccc(C(C)C)c3)c(=O)n21] | TFA | 447.66 | 446.24 |
| 209 | 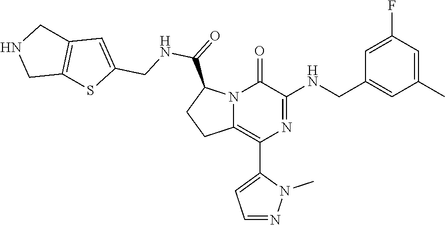 [SMILES: Cc1cc(F)cc(CNc2nc(-c3ccnn3C)c3n(c2=O)[C@H](C(=O)NCc2cc4c(s2)CNC4)CC3)c1] | TFA | 534.61 | 533.20 |
| 210 | 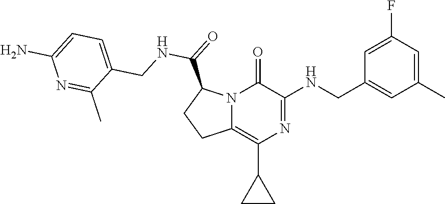 [SMILES: Cc1cc(F)cc(CNc2nc(C3CC3)c3n(c2=O)[C@H](C(=O)NCc2ccc(N)nc2C)CC3)c1] | TFA | 477.64 | 476.23 |
| 211 | 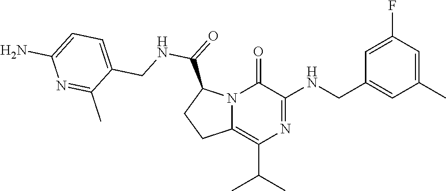 [SMILES: Cc1cc(F)cc(CNc2nc(C(C)C)c3n(c2=O)[C@H](C(=O)NCc2ccc(N)nc2C)CC3)c1] | TFA | 479.64 | 478.25 |
| 212 | 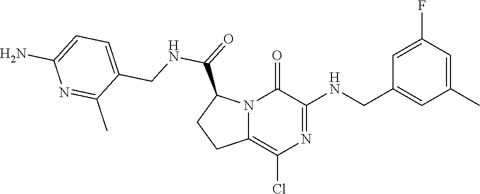 [SMILES: Cc1cc(F)cc(CNc2nc(Cl)c3n(c2=O)[C@H](C(=O)NCc2ccc(N)nc2C)CC3)c1] | TFA | 471.55 | 470.16 |
| 213 | 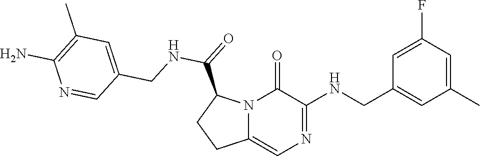 [SMILES: Cc1cc(F)cc(CNc2ncc3n(c2=O)[C@H](C(=O)NCc2cnc(N)c(C)c2)CC3)c1] | TFA | 437.56 | 436.20 |
| 214 | 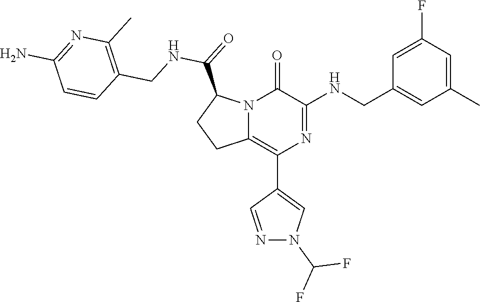 [SMILES: Cc1cc(F)cc(CNc2nc(-c3cnn(C(F)F)c3)c3n(c2=O)[C@H](C(=O)NCc2ccc(N)nc2C)CC3)c1] | TFA | 553.60 | 552.22 |
| 215 | 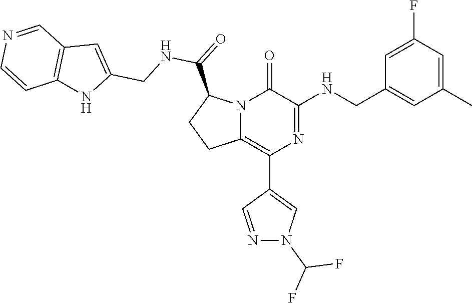 [SMILES: Cc1cc(F)cc(CNc2nc(-c3cnn(C(F)F)c3)c3n(c2=O)[C@H](C(=O)NCc2cc4cnccc4[nH]2)CC3)c1] | TFA | 563.60 | 562.21 |
| 216 | 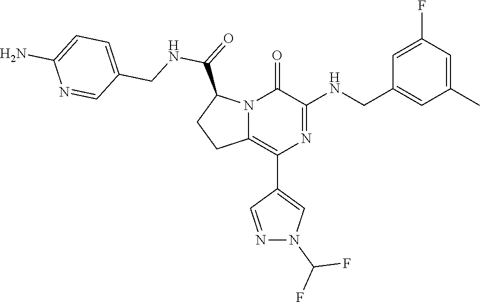 [SMILES: Cc1cc(F)cc(CNc2nc(-c3cnn(C(F)F)c3)c3n(c2=O)[C@H](C(=O)NCc2ccc(N)nc2)CC3)c1] | TFA | 539.51 | 538.21 |
| 217 | 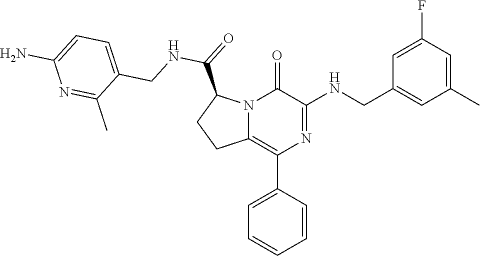 [SMILES: Cc1cc(F)cc(CNc2nc(-c3ccccc3)c3n(c2=O)[C@H](C(=O)NCc2ccc(N)nc2C)CC3)c1] | TFA | 513.53 | 512.23 |
| 218 | 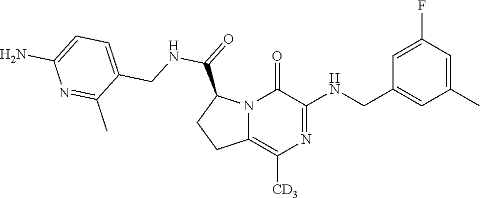 [SMILES: [2H]C([2H])([2H])c1nc(NCc2cc(C)cc(F)c2)c(=O)n2c1CC[C@H]2C(=O)NCc1ccc(N)nc1C] | TFA | 454.56 | 453.24 |
| 219 | 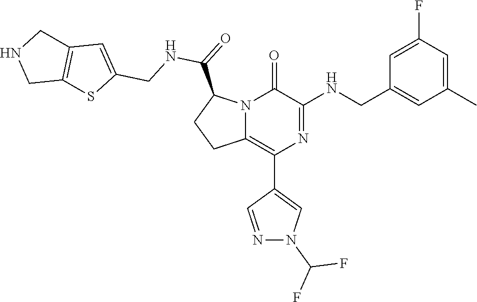 [SMILES: Cc1cc(F)cc(CNc2nc(-c3cnn(C(F)F)c3)c3n(c2=O)[C@H](C(=O)NCc2cc4c(s2)CNC4)CC3)c1] | TFA | 570.49 | 569.18 |
| 220 | 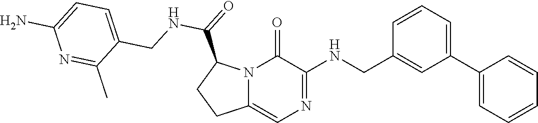 [SMILES: Cc1nc(N)ccc1CNC(=O)[C@@H]1CCc2cnc(NCc3cccc(-c4ccccc4)c3)c(=O)n21] | TFA | 481.64 | 480.23 |
| 221 | 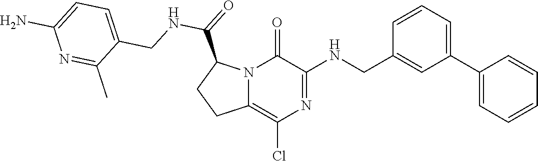 [SMILES: Cc1nc(N)ccc1CNC(=O)[C@@H]1CCc2c(Cl)nc(NCc3cccc(-c4ccccc4)c3)c(=O)n21] | TFA | 515.53 | 514.19 |
| 222 | 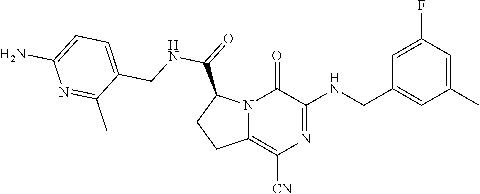 [SMILES: Cc1cc(F)cc(CNc2nc(C#N)c3n(c2=O)[C@H](C(=O)NCc2ccc(N)nc2C)CC3)c1] | TFA | 462.55 | 461.20 |
| 223 | 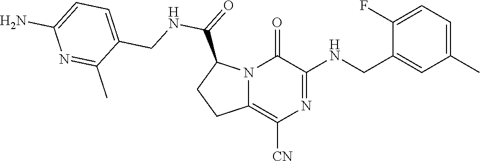 [SMILES: Cc1ccc(F)c(CNc2nc(C#N)c3n(c2=O)[C@H](C(=O)NCc2ccc(N)nc2C)CC3)c1] | TFA | 462.55 | 461.20 |
| 224 | 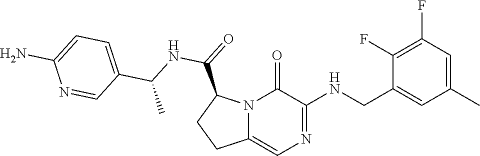 [SMILES: Cc1cc(F)c(F)c(CNc2ncc3n(c2=O)[C@H](C(=O)N[C@H](C)c2ccc(N)nc2)CC3)c1] | TFA | 455.56 | 454.19 |
| 225 | 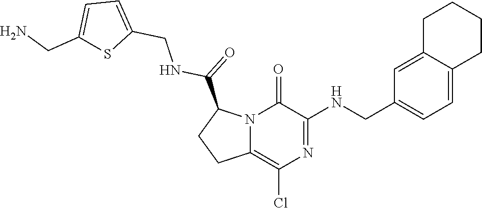 [SMILES: NCc1ccc(CNC(=O)[C@@H]2CCc3c(Cl)nc(NCc4ccc5c(c4)CCCC5)c(=O)n32)s1] | TFA | 498.63 | 497.17 |
| 226 | 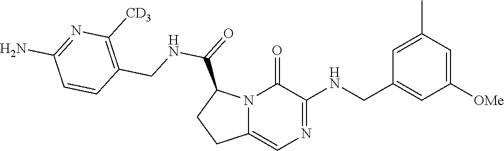 [SMILES: [2H]C([2H])([2H])c1nc(N)ccc1CNC(=O)[C@@H]1CCc2cnc(NCc3cc(C)cc(OC)c3)c(=O)n21] | | 452.25 | 451.24 |
| 227 | 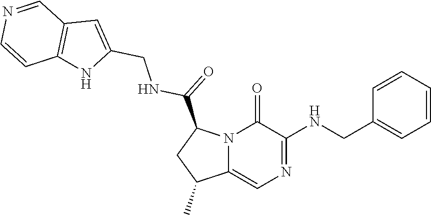 [SMILES: C[C@@H]1C[C@@H](C(=O)NCc2cc3cnccc3[nH]2)n2c1cnc(NCc1ccccc1)c2=O] | TFA | 429.25 | 428.20 |
| 228 | 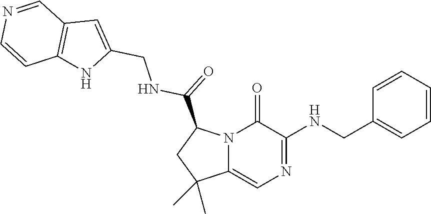 [SMILES: CC1(C)C[C@@H](C(=O)NCc2cc3cnccc3[nH]2)n2c1cnc(NCc1ccccc1)c2=O] | TFA | 443.24 | 442.21 |
| 229 | 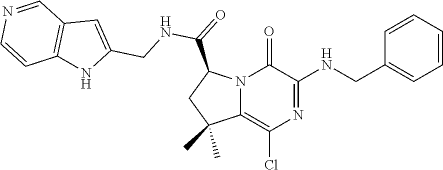 | TFA | 477.74 | 476.17 |
| 230 | 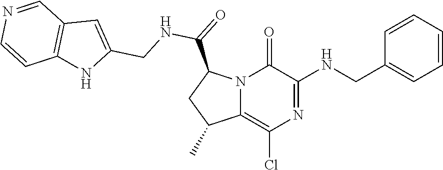 [SMILES: C[C@@H]1C[C@@H](C(=O)NCc2cc3cnccc3[nH]2)n2c1c(Cl)nc(NCc1ccccc1)c2=O] | TFA | 463.60 | 462.16 |
| 231 | 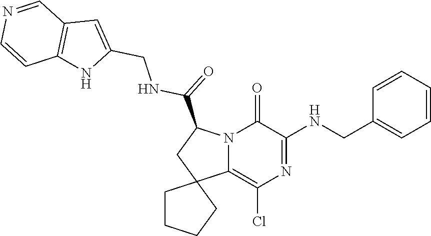 [SMILES: O=C(NCc1cc2cnccc2[nH]1)[C@@H]1CC2(CCCC2)c2c(Cl)nc(NCc3ccccc3)c(=O)n21] | TFA | 503.43 | 502.19 |
| 232 | 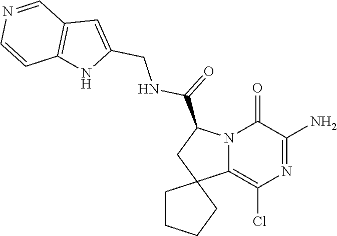 [SMILES: Nc1nc(Cl)c2n(c1=O)[C@H](C(=O)NCc1cc3cnccc3[nH]1)CC21CCCC1] | TFA | 413.39 | 412.14 |
| 233 | 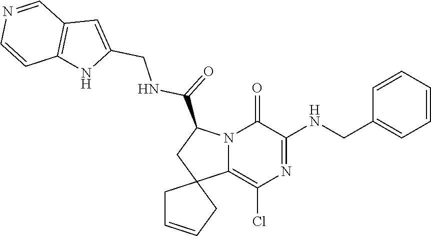 [SMILES: O=C(NCc1cc2cnccc2[nH]1)[C@@H]1CC2(CC=CC2)c2c(Cl)nc(NCc3ccccc3)c(=O)n21] | TFA | 501.20 | 500.17 |
| 234 | 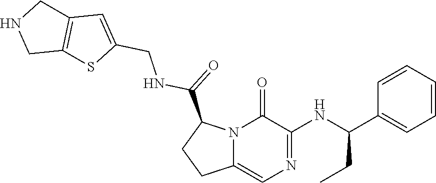 [SMILES: CC[C@@H](Nc1ncc2n(c1=O)[C@H](C(=O)NCc1cc3c(s1)CNC3)CC2)c1ccccc1] | | 450.21 | 449.19 |
| 235 | 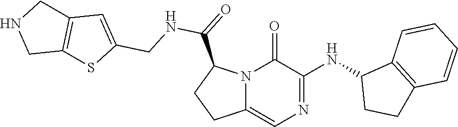 | | 448.20 | 447.17 |
| 236 | 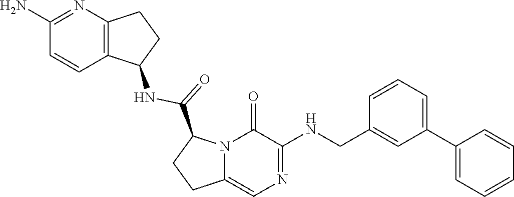 [SMILES: Nc1ccc2c(n1)CC[C@H]2NC(=O)[C@@H]1CCc2cnc(NCc3cccc(-c4ccccc4)c3)c(=O)n21] | TFA | 493.60 | 492.23 |
| 237 | 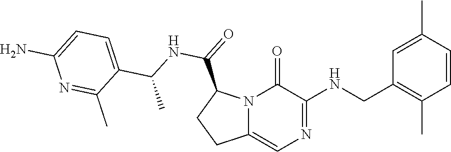 [SMILES: Cc1ccc(C)c(CNc2ncc3n(c2=O)[C@H](C(=O)N[C@H](C)c2ccc(N)nc2C)CC3)c1] | | 447.56 | 446.24 |
| 238 | 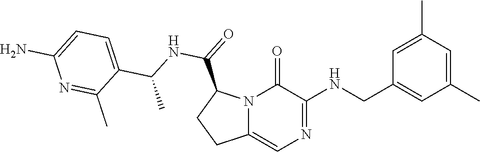 [SMILES: Cc1cc(C)cc(CNc2ncc3n(c2=O)[C@H](C(=O)N[C@H](C)c2ccc(N)nc2C)CC3)c1] | | 447.56 | 446.24 |
| 239 | 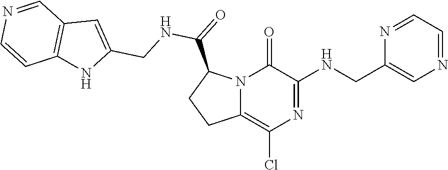 [SMILES: O=C(NCc1cc2cnccc2[nH]1)[C@@H]1CCc2c(Cl)nc(NCc3cnccn3)c(=O)n21] | | 451.41 | 450.13 |
| 240 | 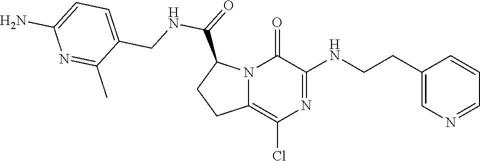 [SMILES: Cc1nc(N)ccc1CNC(=O)[C@@H]1CCc2c(Cl)nc(NCCc3cccnc3)c(=O)n21] | | 454.38 | 453.17 |
| 241 | 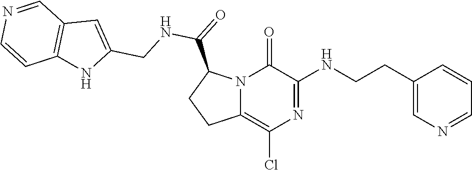 [SMILES: O=C(NCc1cc2cnccc2[nH]1)[C@@H]1CCc2c(Cl)nc(NCCc3cccnc3)c(=O)n21] | | 464.44 | 463.15 |
| 242 | 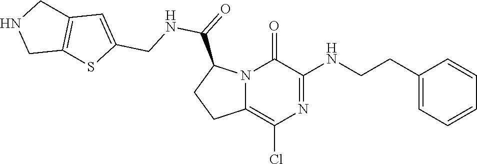 [SMILES: O=C(NCc1cc2c(s1)CNC2)[C@@H]1CCc2c(Cl)nc(NCCc3ccccc3)c(=O)n21] | TFA | 470.37 | 469.13 |
| 243 | 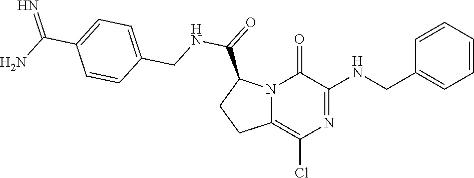 [SMILES: N=C(N)c1ccc(CNC(=O)[C@@H]2CCc3c(Cl)nc(NCc4ccccc4)c(=O)n32)cc1] | | 451.33 | 450.16 |
| 244 | 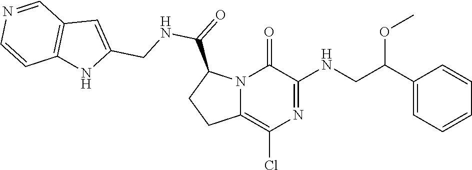 [SMILES: COC(CNc1nc(Cl)c2n(c1=O)[C@H](C(=O)NCc1cc3cnccc3[nH]1)CC2)c1ccccc1] | | 493.02 | 492.17 |
| 245 | 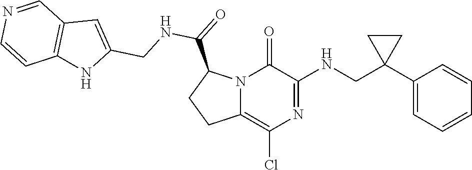 [SMILES: O=C(NCc1cc2cnccc2[nH]1)[C@@H]1CCc2c(Cl)nc(NCC3(c4ccccc4)CC3)c(=O)n21] | TFA | 488.97 | 488.17 |
| 246 | 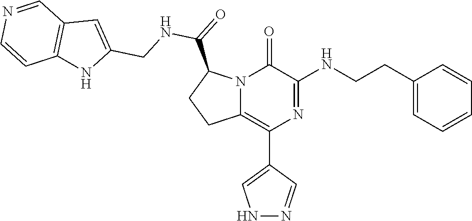 [SMILES: O=C(NCc1cc2cnccc2[nH]1)[C@@H]1CCc2c(-c3cn[nH]c3)nc(NCCc3ccccc3)c(=O)n21] | TFA | 495.29 | 494.22 |
| 247 | 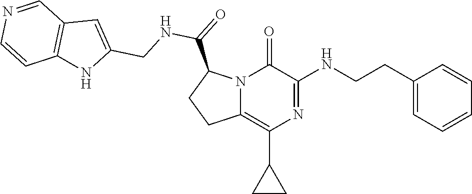 [SMILES: O=C(NCc1cc2cnccc2[nH]1)[C@@H]1CCc2c(C3CC3)nc(NCCc3ccccc3)c(=O)n21] | TFA | 467.12 (ES−; M − H) | 468.23 |
| 248 | 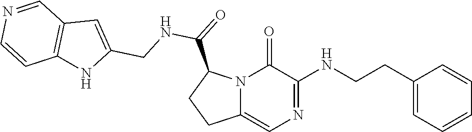 [SMILES: O=C(NCc1cc2cnccc2[nH]1)[C@@H]1CCc2cnc(NCCc3ccccc3)c(=O)n21] | TFA | 429.20 | 428.20 |
| 249 | 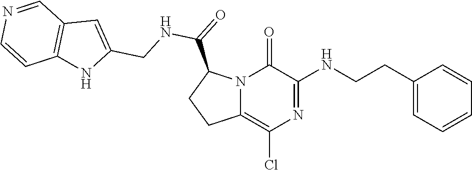 [SMILES: O=C(NCc1cc2cnccc2[nH]1)[C@@H]1CCc2c(Cl)nc(NCCc3ccccc3)c(=O)n21] | | 463.05 | 462.16 |
| 250 | 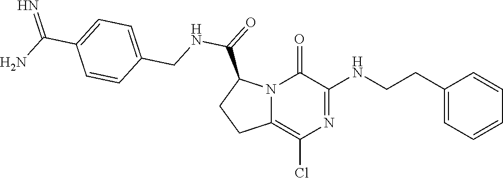 [SMILES: N=C(N)c1ccc(CNC(=O)[C@@H]2CCc3c(Cl)nc(NCCc4ccccc4)c(=O)n32)cc1] | | 465.12 | 464.17 |
| 251 | 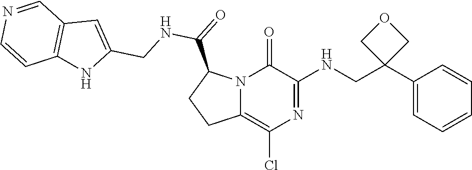 [SMILES: O=C(NCc1cc2cnccc2[nH]1)[C@@H]1CCc2c(Cl)nc(NCC3(c4ccccc4)COC3)c(=O)n21] | TFA | 505.00 | 504.17 |
| 252 | 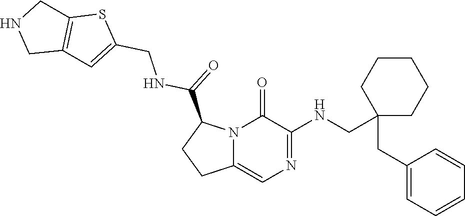 [SMILES: O=C(NCc1cc2c(s1)CNC2)[C@@H]1CCc2cnc(NCC3(Cc4ccccc4)CCCCC3)c(=O)n21] | TFA | 518.21 | 517.25 |
| 253 | 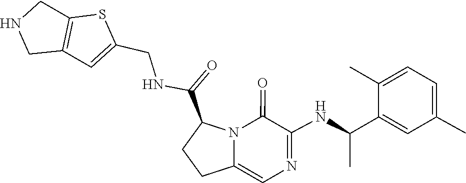 [SMILES: Cc1ccc(C)c([C@@H](C)Nc2ncc3n(c2=O)[C@H](C(=O)NCc2cc4c(s2)CNC4)CC3)c1] | N/A | 464.29 | 463.20 |
| 254 | 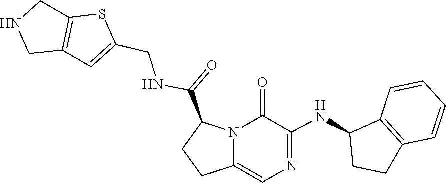 [SMILES: O=C(NCc1cc2c(s1)CNC2)[C@@H]1CCc2cnc(N[C@@H]3CCc4ccccc43)c(=O)n21] | N/A | 448.25 | 447.17 |
| 255 | 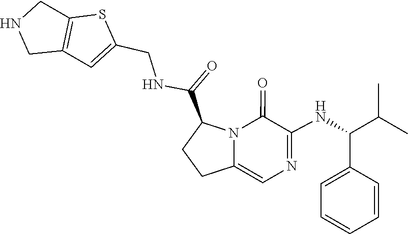 [SMILES: CC(C)[C@@H](Nc1ncc2n(c1=O)[C@H](C(=O)NCc1cc3c(s1)CNC3)CC2)c1ccccc1] | TFA | 464.26 | 463.20 |
| 256 | 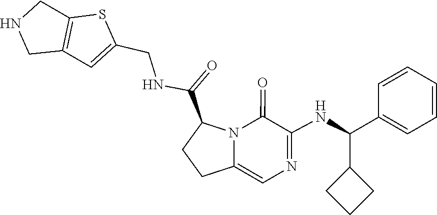 [SMILES: O=C(NCc1cc2c(s1)CNC2)[C@@H]1CCc2cnc(N[C@@H](c3ccccc3)C3CCC3)c(=O)n21] | TFA | 476.26 | 475.20 |
| 257 | 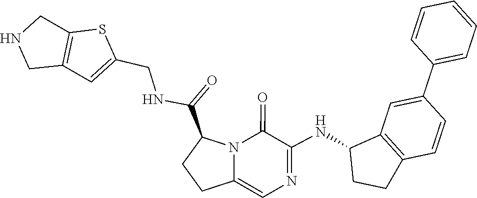 [SMILES: O=C(NCc1cc2c(s1)CNC2)[C@@H]1CCc2cnc(N[C@H]3CCc4ccc(-c5ccccc5)cc43)c(=O)n21] | TFA | 524.29 | 523.20 |
| 258 | 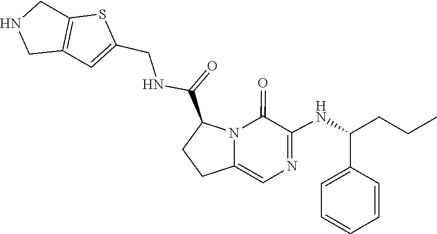 [SMILES: CCC[C@@H](Nc1ncc2n(c1=O)[C@H](C(=O)NCc1cc3c(s1)CNC3)CC2)c1ccccc1] | TFA | 464.25 | 463.20 |
| 259 | 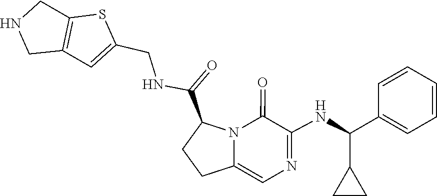 [SMILES: O=C(NCc1cc2c(s1)CNC2)[C@@H]1CCc2cnc(N[C@@H](c3ccccc3)C3CC3)c(=O)n21] | TFA | 462.23 | 461.19 |
| 260 | 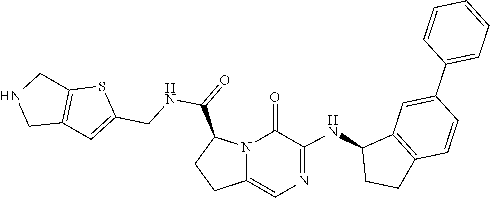 [SMILES: O=C(NCc1cc2c(s1)CNC2)[C@@H]1CCc2cnc(N[C@@H]3CCc4ccc(-c5ccccc5)cc43)c(=O)n21] | TFA | 524.23 | 523.20 |
| 261 | 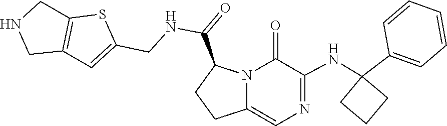 [SMILES: O=C(NCc1cc2c(s1)CNC2)[C@@H]1CCc2cnc(NC3(c4ccccc4)CCC3)c(=O)n21] | TFA | 462.22 | 461.19 |
| 262 | 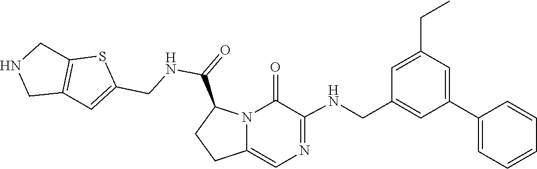 [SMILES: CCc1cc(CNc2ncc3n(c2=O)[C@H](C(=O)NCc2cc4c(s2)CNC4)CC3)cc(-c2ccccc2)c1] | TFA | 526.28 | 525.22 |
| 263 | 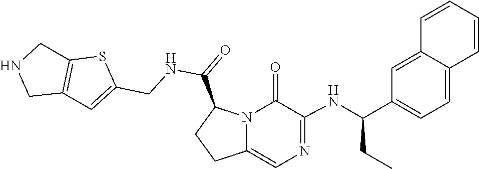 [SMILES: CC[C@@H](Nc1ncc2n(c1=O)[C@H](C(=O)NCc1cc3c(s1)CNC3)CC2)c1ccc2ccccc2c1] | TFA | 500.24 | 499.20 |
| 264 | 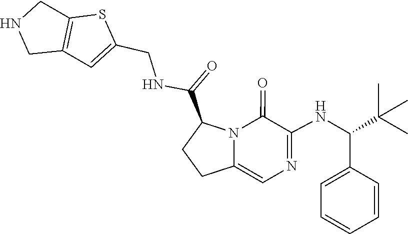 [SMILES: CC(C)(C)[C@@H](Nc1ncc2n(c1=O)[C@H](C(=O)NCc1cc3c(s1)CNC3)CC2)c1ccccc1] | TFA | 478.25 | 477.22 |
| 265 | 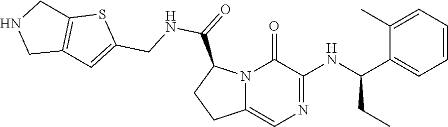 [SMILES: CC[C@@H](Nc1ncc2n(c1=O)[C@H](C(=O)NCc1cc3c(s1)CNC3)CC2)c1ccccc1C] | TFA | 464.28 | 463.20 |
| 266 | 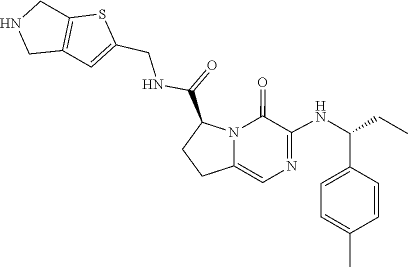 [SMILES: CC[C@@H](Nc1ncc2n(c1=O)[C@H](C(=O)NCc1cc3c(s1)CNC3)CC2)c1ccc(C)cc1] | TFA | 464.28 | 463.20 |
| 267 | 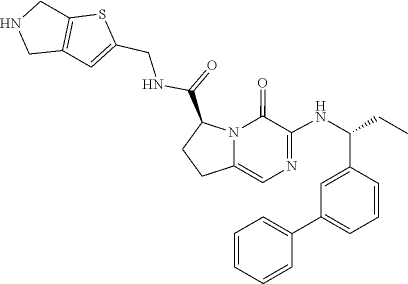 [SMILES: CC[C@@H](Nc1ncc2n(c1=O)[C@H](C(=O)NCc1cc3c(s1)CNC3)CC2)c1cccc(-c2ccccc2)c1] | TFA | 526.21 | 525.22 |
| 268 | 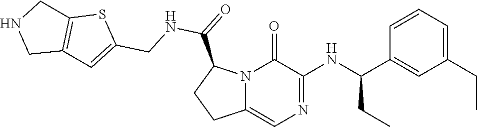 [SMILES: CCc1cccc([C@@H](CC)Nc2ncc3n(c2=O)[C@H](C(=O)NCc2cc4c(s2)CNC4)CC3)c1] | TFA | 478.17 | 477.22 |
| 269 | 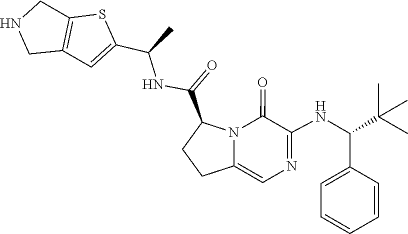 [SMILES: C[C@@H](NC(=O)[C@@H]1CCc2cnc(N[C@@H](c3ccccc3)C(C)(C)C)c(=O)n21)c1cc2c(s1)CNC2] | TFA | 492.22 | 491.24 |
| 270 | 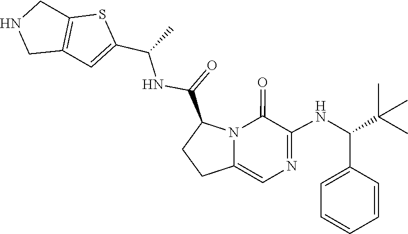 [SMILES: C[C@H](NC(=O)[C@@H]1CCc2cnc(N[C@@H](c3ccccc3)C(C)(C)C)c(=O)n21)c1cc2c(s1)CNC2] | TFA | 492.22 | 491.24 |
| 271 | 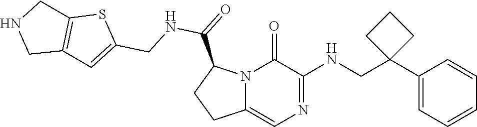 [SMILES: O=C(NCc1cc2c(s1)CNC2)[C@@H]1CCc2cnc(NCC3(c4ccccc4)CCC3)c(=O)n21] | TFA | 476.21 | 475.20 |
| 272 | 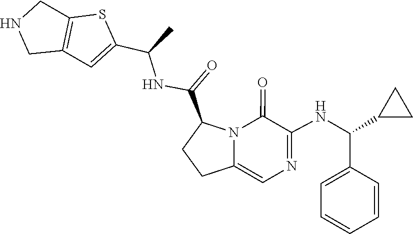 [SMILES: C[C@@H](NC(=O)[C@@H]1CCc2cnc(N[C@@H](c3ccccc3)C3CC3)c(=O)n21)c1cc2c(s1)CNC2] | TFA | 476.40 | 475.20 |
| 273 | 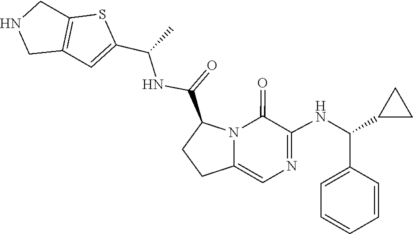 [SMILES: C[C@H](NC(=O)[C@@H]1CCc2cnc(N[C@@H](c3ccccc3)C3CC3)c(=O)n21)c1cc2c(s1)CNC2] | TFA | 476.40 | 475.20 |
| 274 | 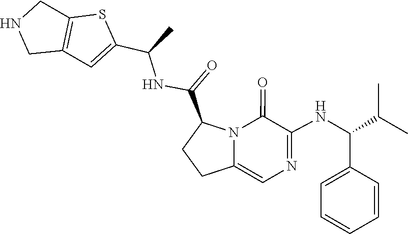 [SMILES: CC(C)[C@@H](Nc1ncc2n(c1=O)[C@H](C(=O)N[C@H](C)c1cc3c(s1)CNC3)CC2)c1ccccc1] | TFA | 478.40 | 477.22 |
| 275 | 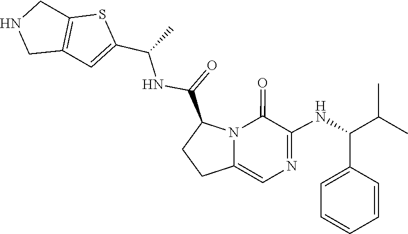 [SMILES: CC(C)[C@@H](Nc1ncc2n(c1=O)[C@H](C(=O)N[C@@H](C)c1cc3c(s1)CNC3)CC2)c1ccccc1] | TFA | 478.40 | 477.22 |
| 276 | 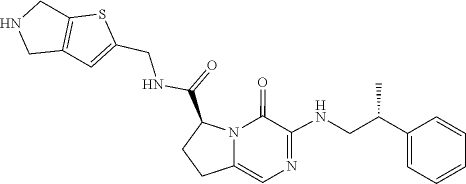 [SMILES: C[C@@H](CNc1ncc2n(c1=O)[C@H](C(=O)NCc1cc3c(s1)CNC3)CC2)c1ccccc1] | TFA | 450.39 | 449.19 |
| 277 | 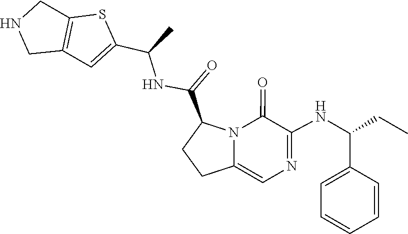 [SMILES: CC[C@@H](Nc1ncc2n(c1=O)[C@H](C(=O)N[C@H](C)c1cc3c(s1)CNC3)CC2)c1ccccc1] | TFA | 464.22 | 463.20 |
| 278 | 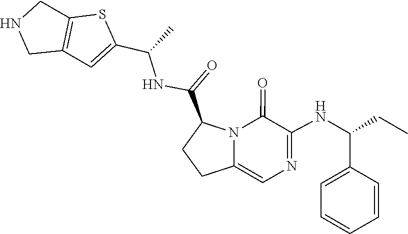 [SMILES: CC[C@@H](Nc1ncc2n(c1=O)[C@H](C(=O)N[C@@H](C)c1cc3c(s1)CNC3)CC2)c1ccccc1] | TFA | 464.22 | 463.20 |
| 279 | 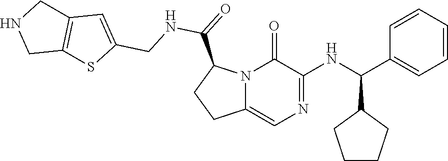 [SMILES: O=C(NCc1cc2c(s1)CNC2)[C@@H]1CCc2cnc(N[C@@H](c3ccccc3)C3CCCC3)c(=O)n21] | N/A | 490.48 | 489.22 |
| 280 | 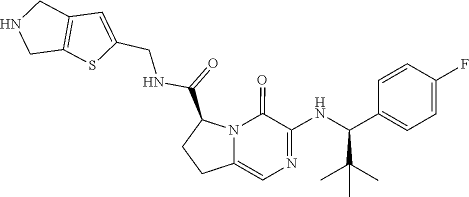 [SMILES: CC(C)(C)[C@@H](Nc1ncc2n(c1=O)[C@H](C(=O)NCc1cc3c(s1)CNC3)CC2)c1ccc(F)cc1] | N/A | 496.43 | 495.21 |
| 281 | 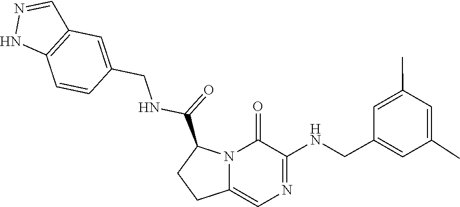 [SMILES: Cc1cc(C)cc(CNc2ncc3n(c2=O)[C@H](C(=O)NCc2ccc4[nH]ncc4c2)CC3)c1] | N/A | 443.40 | 442.21 |
| 282 | 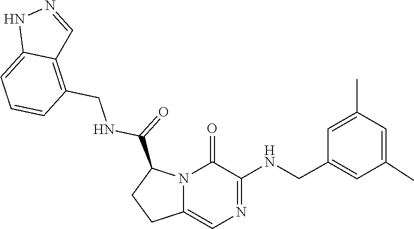 [SMILES: Cc1cc(C)cc(CNc2ncc3n(c2=O)[C@H](C(=O)NCc2cccc4[nH]ncc24)CC3)c1] | N/A | 443.40 | 442.21 |
| 283 | 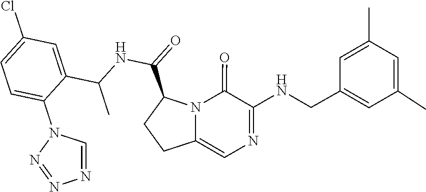 [SMILES: Cc1cc(C)cc(CNc2ncc3n(c2=O)[C@H](C(=O)NC(C)c2cc(Cl)ccc2-n2cnnn2)CC3)c1] | N/A | 519.44 | 518.19 |
| 284 | 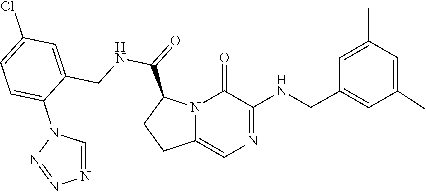 [SMILES: Cc1cc(C)cc(CNc2ncc3n(c2=O)[C@H](C(=O)NCc2cc(Cl)ccc2-n2cnnn2)CC3)c1] | N/A | 505.35 | 504.18 |
| 285 | 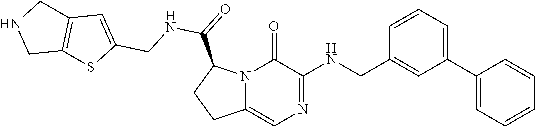 [SMILES: O=C(NCc1cc2c(s1)CNC2)[C@@H]1CCc2cnc(NCc3cccc(-c4ccccc4)c3)c(=O)n21] | TFA | 498.6 | 497.19 |
| 286 | 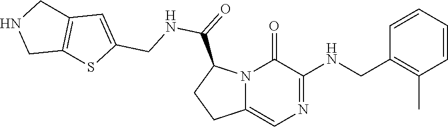 [SMILES: Cc1ccccc1CNc1ncc2n(c1=O)[C@H](C(=O)NCc1cc3c(s1)CNC3)CC2] | TFA | 436.39 | 435.17 |

“Cmp No.” = Compound Number.
“Exact Obs'd. Mass” = Exact Observed mass.
Exact Calc. Mass = Exact Calculated Mass.

### Example 71

### Enzymatic Assay for MASP-2 Inhibitory Activity

The MASP-2 assay utilizes a fluorogenic substrate, based on the cleavage site for its natural substrate C2. The assay was run at room temperature in an assay buffer containing 20 mM HEPES, pH 7.4, 140 mM NaCl and 0.1% Tween 20. Assay parameters were adjusted such that the assay was linear with respect to time, enzyme, and substrate concentrations. Under the optimized assays conditions, IC₅₀ values were equivalent to Kᵢ values, except in the few cases of “tight binding” inhibitors. Cases of “tight binding” or possible “slow binding” inhibitors were handled by the methods described in Copeland R. A. (2013) Evaluation of Enzyme Inhibitors in Drug Discovery. 2nd Ed., John Wiley and Sons, Inc., Chapters 5-7.

The MASP-2 assay protocol was carried out as follows. Test compounds were serially diluted in DMSO and then 100 nL of each dilution was transferred to the assay plate(s). 10 μL of Assay Buffer was added, followed by 15 μL of Enzyme MASP-2 (CCP1-CCP2-SP) in Assay Buffer. 15 μL of Substrate in Assay Buffer was then added and the mixture was mixed to start the reaction. After 20 min at room temperature, 15 μL of a stop solution (0.1 M acetic acid) was added, the sample was mixed, and the plates were read on a SpectraMax i3x Microplate Reader with the data exported as an Excel file. Each assay plate included a “no inhibitor” (DMSO only) control, a “no enzyme” control, and a reference inhibitor control. % Activity values=100*(average test comp. fluorescence−average “no enzyme” fluorescence)/(average “DMSO only” fluorescence−average “no enzyme” fluorescence). IC₅₀ and Kᵢ replicate values were very reproducible, with values well within ±2-fold.

The biological assay results for the compounds listed in Table 1 are shown in Table 2, below.

### Example 72

### Enzymatic Assay for Thrombin

The thrombin assay utilizes a fluorogenic peptide substrate (Boc-VPR-AMC (R&D Systems) and was run at room temperature in an assay buffer containing 20 mM Hepes, pH 7.4, 140 mM NaCl and 0.1% Tween 20. Assay parameters were adjusted such that the assay was linear with respect to time, enzyme, and substrate concentrations. Under these optimized assays conditions, IC₅₀ values were equivalent to Kᵢ values, except in a few cases of “tight binding” inhibitors. Cases of “tight binding” or possible “slow binding” inhibitors were handled by the methods described in Copeland R. A. (2013) Evaluation of Enzyme Inhibitors in Drug Discovery. 2nd Ed. John Wiley and Sons, Inc., Chapters 5-7.

The thrombin assay protocol was carried out as follows. Test compounds were serially diluted in DMSO and then 100 nL of each dilution was transferred to the assay plate(s). 10 μL of Assay Buffer was added, followed by 15 μL of enzyme (human α-thrombin (BioPharm® Lab.)) in assay buffer. 15 μL of substrate in assay buffer was then added and the sample mixed to start the reaction. After 20 min at room temperature, 15 μL of a stop solution (0.1 M acetic acid) was added, the sample was mixed, and the plates were read on a SpectraMax® i3x Microplate Reader with the data exported as Excel files. Each assay plate included a “no inhibitor” (DMSO only) control, a “no enzyme” control, and a reference inhibitor control. % Activity values=100*(ave. test comp. fluorescence−ave. “no enz” fluorescence)/(ave. “DMSO only” fluorescence−ave. “no enz” fluorescence). IC₅₀ and Kᵢ values were very reproducible, with values falling well within ±2-fold.

The biological assay results for the compounds listed in Table 1 are shown in Table 2, below.

### Example 73

Lectin Pathway Activation (LPA) Assay in Human Serum Treated with Compounds

A microtiter ELISA plate was coated with mannan (1 mg/ml) from Saccharomyces cerevisiae (Sigma-Aldrich®, M7504) overnight at 4° C. in coating buffer [33.3 mM Na₂CO₃, 166.67 mM NaHCO₃]. The plate was blocked with 5% bovine serum albumin (BSA) (Sigma-Aldrich®, A3294) in 1×PBST [Phosphate Buffered Saline Solution (1.06 mM KH₂PO₄, 155.2 mM NaCl, 2.97 mM Na₂HPO₄⁻·7H₂O, pH 7.4), 0.05% Tween-20] shaking for 2 hours at room temperature. 1% human serum was incubated with serial dilutions of small compounds in 1× LPA+ Buffer [20 mM Hepes, pH 7.4, 140 mM NaCl, 2 mM MgCl₂ (6H₂O)] and incubated for 5 minutes at room temperature. 40 μL of this mixture was then added to the plate, and the plate was incubated at room temperature for 55 mins. Next, the reaction was stopped by adding 20 μL of EDTA Stop Solution [20 mM Hepes, pH7.4, 140 mM NaCl, 150 mM EDTA] to the plate. The plate was then washed 10× in wash buffer 1×PBST; 40 μL of Rabbit Anti Human C4c Antibody [Dako®, Q0369] diluted 1:20000 in wash buffer was added; and the plate was incubated shaking at room temperature for 30 minutes. The plate was washed [5× times]; 40 μL of Goat Anti-rabbit Antibody-HRP Conjugate [Southern Biotech®, 4050-05] diluted 1:10000 in wash buffer was added; and the plate was incubated shaking at room temperature for 30 minutes. After that, the plate was washed five times; 40 L/well of Amplex® UltraRed Substrate Solution [1× LPA Buffer, 10 mM Amplex® UltraRed Stock Solution [Thermo Fisher Sci.®, A36006], 3% stabilized hydrogen peroxide [Sigma Aldrich®, 216763] was added; the plate was incubated at room temperature for 16 minutes; the reaction was stopped by adding 8 L/well of Amplex® UltraRed Stop Solution [Thermo Fisher Sci.®, A33855] in 100% EtOH. Finally, the plate was read for fluorescence at Ex: 550 nm, Em: 580 nm on the i3x Microplate Reader.

The biological assay results for the compounds listed in Table 1 are shown in Table 3, below.

### Example 74

### Biological Data for Exemplary Compounds

Biological assay results for the compounds listed in Table 1 are shown in Table 2.

TABLE 2
MASP-2 and Thrombin Inhibition by Exemplary
Compounds of the Disclosure

| Cmp No. | MASP-2 Kᵢ (μM) | Thrombin Kᵢ (μM) |
|---|---|---|
| 1 | **** | * |
| 2 | **** | ** |
| 3 | **** | ** |
| 4 | **** | *** |
| 5 | **** | *** |
| 6 | **** | ** |
| 7 | **** | ** |
| 8 | **** | *** |
| 9 | **** | ** |
| 10 | **** | *** |
| 11 | **** | * |
| 12 | **** | ** |
| 13 | **** | * |
| 14 | **** | −− |
| 15 | **** | ** |
| 16 | **** | ** |
| 17 | **** | * |
| 18 | **** | ** |
| 19 | **** | −− |
| 20 | **** | *** |
| 21 | **** | ** |
| 22 | **** | * |
| 23 | **** | −− |
| 24 | **** | ** |
| 25 | **** | * |
| 26 | **** | * |
| 27 | **** | ** |
| 28 | **** | ** |
| 29 | **** | * |
| 30 | **** | ** |
| 31 | **** | ** |
| 32 | **** | ** |
| 33 | **** | ** |
| 34 | **** | ** |
| 35 | **** | ** |
| 36 | **** | **** |
| 37 | **** | **** |
| 38 | **** | **** |
| 39 | **** | ** |
| 40 | * | −− |
| 41 | **** | *** |
| 42 | **** | * |
| 43 | **** | −− |
| 44 | **** | *** |
| 45 | **** | **** |
| 46 | **** | **** |
| 47 | **** | **** |
| 48 | **** | **** |
| 49 | **** | **** |
| 50 | **** | * |
| 51 | **** | ** |
| 52 | **** | **** |
| 53 | **** | *** |
| 54 | **** | *** |
| 55 | **** | **** |
| 56 | **** | *** |
| 57 | **** | * |
| 58 | **** | −− |
| 59 | **** | *** |
| 60 | **** | **** |
| 61 | **** | ** |
| 62 | **** | **** |
| 63 | **** | *** |
| 64 | **** | * |
| 65 | **** | **** |
| 66 | **** | **** |
| 67 | **** | *** |
| 68 | **** | −− |
| 69 | **** | **** |
| 70 | **** | *** |
| 71 | **** | * |
| 72 | **** | **** |
| 73 | **** | * |
| 74 | **** | *** |
| 75 | **** | **** |
| 76 | **** | **** |
| 77 | **** | **** |
| 78 | **** | **** |
| 79 | **** | **** |
| 80 | **** | * |
| 81 | **** | **** |
| 82 | **** | **** |
| 83 | **** | ** |
| 84 | *** | * |
| 85 | **** | −− |
| 86 | *** | −− |
| 87 | **** | **** |
| 88 | **** | **** |
| 89 | ** | −− |
| 90 | **** | ** |
| 91 | **** | **** |
| 92 | **** | **** |
| 93 | **** | **** |
| 94 | **** | *** |
| 95 | **** | *** |
| 96 | **** | **** |
| 97 | **** | *** |
| 98 | **** | *** |
| 99 | **** | **** |
| 100 | **** | −− |
| 101 | **** | −− |
| 102 | **** | *** |
| 103 | **** | −− |
| 104 | **** | **** |
| 105 | **** | * |
| 106 | **** | ** |
| 107 | *** | **** |
| 108 | **** | * |
| 109 | **** | ** |
| 110 | **** | ** |
| 111 | **** | ** |
| 112 | **** | −− |
| 113 | **** | ** |
| 114 | **** | ** |
| 115 | **** | *** |
| 116 | **** | *** |
| 117 | **** | ** |
| 118 | **** | * |
| 119 | ** | −− |
| 120 | **** | −− |
| 121 | **** | * |
| 122 | **** | **** |
| 123 | **** | *** |
| 124 | **** | −− |
| 125 | **** | ** |
| 126 | **** | *** |
| 127 | **** | ** |
| 128 | **** | ** |
| 129 | **** | * |
| 130 | **** | −− |
| 131 | **** | *** |
| 132 | **** | −− |
| 133 | **** | ** |
| 134 | **** | *** |
| 135 | **** | ** |
| 136 | **** | *** |
| 137 | **** | −− |
| 138 | **** | **** |
| 139 | **** | * |
| 140 | **** | **** |
| 141 | **** | *** |
| 142 | **** | ** |
| 143 | **** | −− |
| 144 | **** | ** |
| 145 | **** | −− |
| 146 | *** | * |
| 147 | **** | ** |
| 148 | **** | *** |
| 149 | **** | ** |
| 150 | **** | ** |
| 151 | **** | *** |
| 152 | **** | ** |
| 153 | **** | ** |
| 154 | **** | **** |
| 155 | **** | **** |
| 156 | **** | **** |
| 157 | **** | **** |
| 158 | **** | ** |
| 159 | **** | * |
| 160 | **** | −− |
| 161 | **** | *** |
| 162 | **** | −− |
| 163 | **** | **** |
| 164 | **** | ** |
| 165 | * | −− |
| 166 | **** | *** |
| 167 | **** | *** |
| 168 | *** | ** |
| 169 | **** | **** |
| 170 | **** | **** |
| 171 | **** | **** |
| 172 | **** | **** |
| 173 | ** | −− |
| 174 | **** | −− |
| 175 | **** | *** |
| 176 | **** | * |
| 177 | **** | −− |
| 178 | **** | −− |
| 179 | **** | **** |
| 180 | *** | ** |
| 181 | **** | ** |
| 182 | **** | **** |
| 183 | *** | ** |
| 184 | *** | *** |
| 185 | ** | ** |
| 186 | **** | **** |
| 187 | **** | ** |
| 188 | **** | **** |
| 189 | **** | *** |
| 190 | — | ** |
| 191 | **** | ** |
| 192 | **** | *** |
| 193 | **** | ** |
| 194 | **** | ** |
| 195 | **** | ** |
| 196 | **** | * |
| 197 | **** | −− |
| 198 | **** | ** |
| 199 | **** | ** |
| 200 | **** | ** |
| 201 | **** | −− |
| 202 | **** | * |
| 203 | **** | ** |
| 204 | ** | −− |
| 205 | **** | ** |
| 206 | *** | * |
| 207 | **** | **** |
| 208 | **** | **** |
| 209 | **** | ** |
| 210 | **** | *** |
| 211 | **** | **** |
| 212 | **** | ** |
| 213 | **** | **** |
| 214 | **** | −− |
| 215 | **** | −− |
| 216 | **** | −− |
| 217 | **** | −− |
| 218 | **** | **** |
| 219 | **** | −− |
| 220 | **** | **** |
| 221 | **** | ** |
| 222 | **** | ** |
| 223 | **** | ** |
| 224 | **** | ** |
| 225 | **** | −− |
| 226 | **** | **** |
| 227 | **** | ** |
| 228 | **** | ** |
| 229 | **** | ** |
| 230 | **** | *** |
| 231 | **** | * |
| 232 | *** | −− |
| 233 | **** | −− |
| 234 | **** | **** |
| 235 | **** | **** |
| 236 | **** | *** |
| 237 | **** | *** |
| 238 | **** | *** |
| 239 | **** | −− |
| 240 | **** | **** |
| 241 | **** | **** |
| 242 | **** | **** |
| 243 | **** | **** |
| 244 | **** | *** |
| 245 | **** | *** |
| 246 | **** | −− |
| 247 | **** | *** |
| 248 | **** | **** |
| 249 | **** | *** |
| 250 | *** | ** |
| 251 | **** | *** |
| 252 | **** | ** |
| 253 | **** | **** |
| 254 | **** | *** |
| 255 | **** | **** |
| 256 | **** | **** |
| 257 | **** | **** |
| 258 | **** | **** |
| 259 | **** | **** |
| 260 | **** | ** |
| 261 | **** | **** |
| 262 | **** | *** |
| 263 | **** | *** |
| 264 | **** | **** |
| 265 | **** | **** |
| 266 | **** | **** |
| 267 | **** | *** |
| 268 | **** | **** |
| 269 | **** | ** |
| 270 | ** | * |
| 271 | **** | **** |
| 272 | **** | ** |
| 273 | ** | −− |
| 274 | **** | *** |
| 275 | **** | −− |
| 276 | **** | **** |
| 277 | **** | *** |
| 278 | **** | * |
| 279 | **** | **** |
| 280 | **** | **** |
| 281 | **** | *** |
| 282 | *** | ** |
| 283 | **** | **** |
| 284 | **** | **** |
| 285 | **** | *** |
| 286 | **** | *** |

MASP-2 and Thrombin Inhibition Kᵢ Values:
* Kᵢ between 10-25 μM
** Kᵢ between 2.5-10 μM
*** Kᵢ between 0.5-2.5 μM
**** Kᵢ less than 0.5 μM
−− Kᵢ greater than 25 μM

### Example 75

### Biological Data for Exemplary Compounds

Biological Lectin Pathway Activation (LPA) assay results for selected compounds listed in Table 1 are shown in Table 3.

TABLE 3
LPA Assay Data for Selected Exemplary
Compounds of the Disclosure

| | LPA | | LPA | | LPA |
|---|---|---|---|---|---|
| | Functional | | Functional | | Functional |
| | Activity | | Activity | | Activity |
| Cmp No. | (IC₅₀, μM) | Cmp No. | (IC₅₀, μM) | Cmp No. | (IC₅₀, μM) |
| 276 | ++++ | 181 | +++ | 27 | ++++ |
| 277 | +++ | 140 | ++++ | 228 | ++++ |
| 259 | ++++ | 71 | ++++ | 230 | +++ |
| 257 | ++++ | 69 | ++++ | 41 | ++++ |
| 197 | ++++ | 226 | ++++ | 88 | +++ |
| 194 | ++++ | 239 | +++ | 131 | ++++ |
| 221 | ++++ | 240 | +++ | 120 | ++++ |
| 78 | ++++ | 155 | ++++ | 75 | ++++ |
| 213 | +++ | 28 | ++++ | 48 | ++++ |

LPA Inhibition IC₅₀ Values:
+++ IC₅₀ value in the range of 0.05 μM to 0.5 μM
++++ IC₅₀ value of less than 0.05 μM

While illustrative embodiments have been illustrated and described, it will be appreciated that various changes can be made therein without departing from the spirit and scope of the invention.

1. A compound of Formula (I): 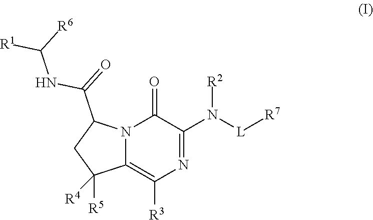
or a stereoisomer, tautomer, or pharmaceutically acceptable salt thereof, wherein:
R¹ is selected from a substituted or unsubstituted C₆-C₁₀ aryl and a substituted or unsubstituted 5-10-membered heteroaryl consisting of a 5-10-membered monocyclic or bicyclic ring system comprising at least one aromatic ring and one to six heteroatoms selected from the group consisting of nitrogen, oxygen, and sulfur, or
R¹ and R⁶, together with the carbon to which they are attached, form a C₃-C₆ cycloalkyl fused to a 5-10-membered heteroaryl or C₆-C₁₀ aryl, wherein the 5-10-membered heteroaryl or C₆-C₁₀ aryl, of the C₃-C₆ cycloalkyl fused to the 5-10-membered heteroaryl or C₆-C₁₀ aryl, is substituted or unsubstituted, and wherein the C₃-C₆ cycloalkyl, of the C₃-C₆ cycloalkyl fused to a 5-10-membered heteroaryl or C₆-C₁₀ aryl, is substituted or unsubstituted;
R² is hydrogen or C₁-C₃ alkyl, or R² and L, together with the nitrogen to which they are attached, form a substituted or unsubstituted 3-6-membered heterocyclyl;
L is —(CR^{8a}R^{8b})ₙ—, wherein each —(CR^{8a}R^{8b})— is the same or is different;
R³ is selected from the group consisting of hydrogen, substituted or unsubstituted C₁-C₃ alkyl, substituted or unsubstituted C₃-C₆ cycloalkyl, substituted or unsubstituted 5-6-membered saturated or unsaturated heterocyclyl, substituted or unsubstituted phenyl, substituted or unsubstituted 5-6-membered heteroaryl, halogen, and cyano;
R⁴ and R⁵ are each independently selected from the group consisting of hydrogen, substituted or unsubstituted C₁-C₆ alkyl, and substituted or unsubstituted benzyl, or R⁴ and R⁵, together with the carbon to which they are attached, form a substituted or unsubstituted C₃-C₆ cycloalkyl or a substituted or unsubstituted 5-6-membered cycloalkenyl;
R⁶ is selected from the group consisting of hydrogen, C₁-C₃ alkyl, C₃-cycloalkyl, and cyano;
R⁷ is selected from the group consisting of hydrogen, substituted or unsubstituted C₁-C₃ alkyl, substituted or unsubstituted 5-6-membered heteroalkyl, substituted or unsubstituted C₃-C₆ cycloalkyl, substituted or unsubstituted C₃-C₆ cycloalkyl fused with aryl, substituted or unsubstituted 3-6-membered heterocyclyl, substituted or unsubstituted C₆-C₁₀ aryl, substituted or unsubstituted C₆-C₁₀ aryl fused with C₅-C₆ cycloalkyl, substituted or unsubstituted 5-9-membered heteroaryl, substituted or unsubstituted 5-9-membered heteroaryl fused with C₅-C₆ cycloalkyl, alkoxy, and halogen, and when R⁷ is hydrogen, n is 0;
R^{8a} and R^{8b} are each independently selected from the group consisting of hydrogen, C₁-C₄ linear alkyl, C₃-C₄ branched alkyl, substituted or unsubstituted C₃-C₆ cycloalkyl, hydroxyalkyl, and alkoxy; or R^{8a} and R^{8b} together, with the carbon to which they are attached, form a substituted or unsubstituted C₃-C₆ cycloalkyl or a substituted or unsubstituted 3-6-membered heterocyclyl; or
n is 3, one of R^{8a} or R^{8b} from a first carbon is a bridging —CH₂— to a third carbon, and one of R^{8a} or R^{8b} from the third carbon is absent, to form a 1,3-cyclobutylene; and
n is 0, 1, 2, 3, or 4, and
wherein when one or more chiral center is present, each chiral center has (R) stereochemistry, (S) stereochemistry, or is a mixture thereof.

2. The compound of claim 1, wherein at least one of R³, R⁴, and R⁵ is not hydrogen, and R³ is not Cl when R¹ is

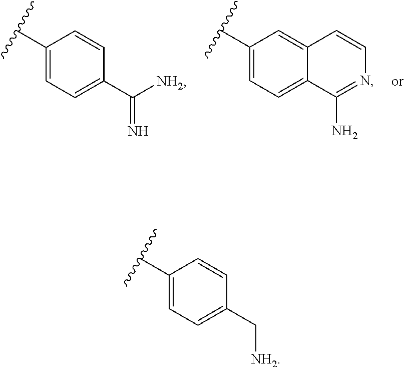 [SMILES: *c1ccc(C(=N)N)cc1 ; *c1ccc2c(N)nccc2c1 ; *c1ccc(CN)cc1]

3. The compound of claim 1, wherein the compound is selected from Formulae (I-1), (I-2), (I-3), (I-4), (I-5), (I-6), (I-7), (I-8), or a combination thereof, wherein:

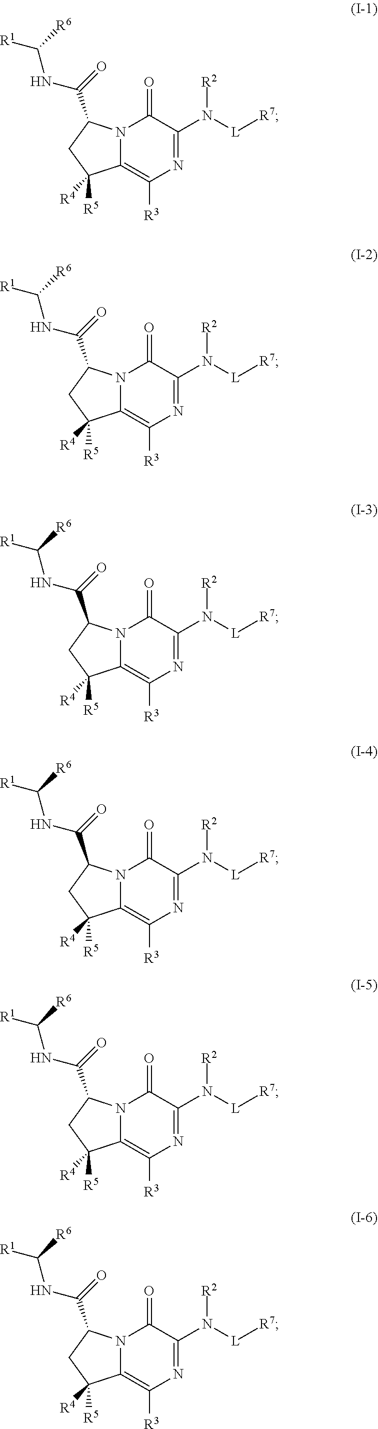

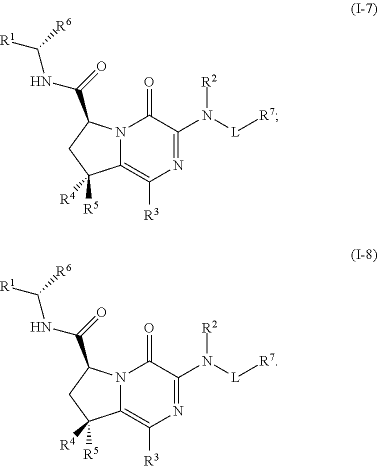

4. (canceled)

5. (canceled)

6. The compound of claim 1, wherein R⁶ is methyl or hydrogen.

7. (canceled)

8. (canceled)

9. The compound of claim 1, wherein R⁶ and R¹, together with the carbon to which they are attached, form a C₃-C₆ cycloalkyl fused to a substituted or unsubstituted 5-10-membered heteroaryl, or a C₃-C₆ cycloalkyl fused to a substituted or unsubstituted C₆-C₁₀ aryl,
wherein the C₃-C₆ cycloalkyl is unsubstituted, or is substituted with C₁-C₃ alkyl, alkoxy, or halogen, and wherein the compound has Formula (I-B): 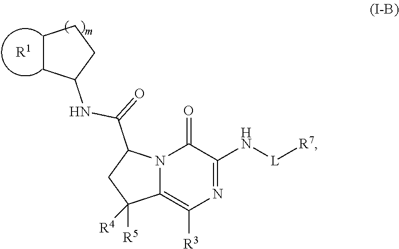
wherein m is 1 or 2.

10.-12. (canceled)

13. The compound of claim 1, wherein R¹ is unsubstituted or substituted C₆-C₁₀ aryl, wherein the substituted C₆-C₁₀ aryl is substituted with one or more of R^{1A}, R^{1B}, R^{1C}, R^{1D}, and R^{1E}, wherein R^{1A}, R^{1B}, R^{1C}, R^{1D}, and R^{1E} are each independently selected from the group consisting of hydrogen, C(═NH)NH₂, C(═NH)NHC(═O)OR⁸, C(═NOC(═O)R⁸)NH₂, C(═NOC(═O)OR⁸)NH₂, C(═NOH)NH₂, C(═NH)NHC(═O)NHC(═O)N(CH₃)R¹³, C₁-C₆ alkyl, C₂-C₆ alkenyl, C₂-C₆ alkynyl, halogen, C₁-C₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃-C₁₀ cycloalkyl, and substituted or unsubstituted 4-10-membered heterocyclyl,
wherein R⁸, R⁹, R¹⁰, R¹¹, and R¹², are, at each occurrence, independently selected from the group consisting of hydrogen, deuterium, C₁-C₆ alkyl, C₂-C₆ alkenyl, C₂-C₆ alkynyl, hydroxyl, C₁-C₆ alkoxy, aryl, arylalkyl, C₁-C₆ haloalkyl, C₁-C₆ haloalkoxy, C₁-C₆ hydroxyalkyl, C₃-C₁₀ cycloalkyl, heterocyclyl, heteroarylalkyl, and heteroaryl,
wherein R¹³ is a C₆-C₁₀ aryl or 5-10-membered heteroaryl, unsubstituted or substituted with CH₂OC(═O)CH₃,
wherein when any one of R^{1A}, R^{1B}, R^{1C}, R^{1D}, and R^{1E} is a substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃-C₁₀ cycloalkyl, or a substituted 4-10 membered heterocyclyl, the R^{1A}, R^{1B}, R^{1C}, R^{1D}, or R^{1E} is unsubstituted or substituted with one or more substituents selected from the group consisting of halo, CN, OR^{e}, SR^{e}, C(O)R^{e}, C(O)NR^{e}R^{f}, C(O)OR^{e}, OC(O)R^{e}, OC(O)NR^{e}R^{f}, NR^{e}R^{f}, NR^{e}C(O)R^{f}, NR^{e}C(O)NR^{f}R^{g}, NR^{e}C(O)OR^{f}, C(═NR^{e})NR^{f}R^{g}, NR^{e}C(═NR^{f})NR^{g}R^{h}, S(O)R^{e}, S(O)NR^{e}R^{f}, S(O)₂R^{e}, NR^{e}S(O)₂R^{f}, S(O)₂NR^{e}R^{f} and oxo, wherein R^{e}, R^{f}, R^{g}, and R^{h} are, at each occurrence, independently selected from the group consisting of hydrogen, C₁-C₆ alkyl, C₂-C₆ alkenyl, C₂-C₆ alkynyl, hydroxyl, C₁-C₆ alkoxy, aryl, arylalkyl, C₁-C₆ haloalkyl, C₁-C₆ haloalkoxy, C₁-C₆ hydroxyalkyl, C₃-C₁₀ cycloalkyl, heterocyclyl, and heteroaryl.

14.-18. (canceled)

19. The compound of claim 1, wherein R¹ is an unsubstituted or substituted 5-10-membered heteroaryl, wherein the substituted 5-10-membered heteroaryl is substituted with one or more of R^{1A}, R^{1B}, R^{1C}, R^{1D}, and R^{1E}, and wherein the R^{1A}, R^{1B}, R^{1C}, R^{1D}, and R^{1E} are each independently selected from the group consisting of hydrogen, C(NH)NH₂, C(═NH)NHC(═O)OR⁸, C(═NOC(═O)R⁸)NH₂, C(═NOC(═O)OR⁸)NH₂, C(═NOH)NH₂, C(═NH)NHC(═O)NHC(═O)N(CH₃)R¹³, C₁-C₆ alkyl, C₂-C₆ alkenyl, C₂-C₆ alkynyl, halogen, C₁-C₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃-C₁₀ cycloalkyl, and substituted or unsubstituted 4-10 membered heterocyclyl, wherein the C₃-C₁₀ cycloalkyl and 4-10 membered heterocyclyl can be a fused, bridge, or spiro ring system,
wherein R⁸, R⁹, R¹⁰, R¹¹, and R¹², are, at each occurrence, independently selected from the group consisting of hydrogen, deuterium, C₁-C₆ alkyl, C₂-C₆ alkenyl, C₂-C₆ alkynyl, hydroxyl, C₁-C₆ alkoxy, aryl, arylalkyl, C₁-C₆ haloalkyl, C₁-C₆ haloalkoxy, C₁-C₆ hydroxyalkyl, C₃-C₁₀ cycloalkyl, heterocyclyl, heteroarylalkyl, and heteroaryl,
wherein R¹³ is a C₆-C₁₀ aryl or 5-10-membered heteroaryl, unsubstituted or substituted with CH₂OC(═O)CH₃,
wherein when any one of R^{1A}, R^{1B}, R^{1C}, R^{1D}, and R^{1E} is a substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃-C₁₀ cycloalkyl, or a substituted 4-10 membered heterocyclyl, the R^{1A}, R^{1B}, R^{1C}, R^{1D}, or R^{1E} is unsubstituted or substituted with one or more substituents selected from the group consisting of halo, CN, OR^{e}, SR^{e}, C(O)R^{e}, C(O)NR^{e}R^{f}, C(O)OR^{e}, OC(O)R^{e}, OC(O)NR^{e}R^{f}, NR^{e}R^{f}, NR^{e}C(O)R^{f}, NR^{e}C(O)NR^{f}R^{g}, NR^{e}C(O)OR^{f}, C(═NR^{e})NR^{f}R^{g}, NR^{e}C(═NR^{f})NR^{g}R^{h}, S(O)R^{e}, S(O)NR^{e}R^{f}, S(O)₂R^{e}, NR^{e}S(O)₂R^{f}, S(O)₂NR^{e}R^{f} and oxo, wherein R^{e}, R^{f}, R^{g}, and R^{h} are, at each occurrence, independently selected from the group consisting of hydrogen, C₁-C₆ alkyl, C₂-C₆ alkenyl, C₂-C₆ alkynyl, hydroxyl, C₁-C₆ alkoxy, aryl, arylalkyl, C₁-C₆ haloalkyl, C₁-C₆ haloalkoxy, C₁-C₆ hydroxyalkyl, C₃-C₁₀ cycloalkyl, heterocyclyl, and heteroaryl.

20.-24. (canceled)

25. The compound of claim 1, wherein R¹ is selected from the group consisting of:

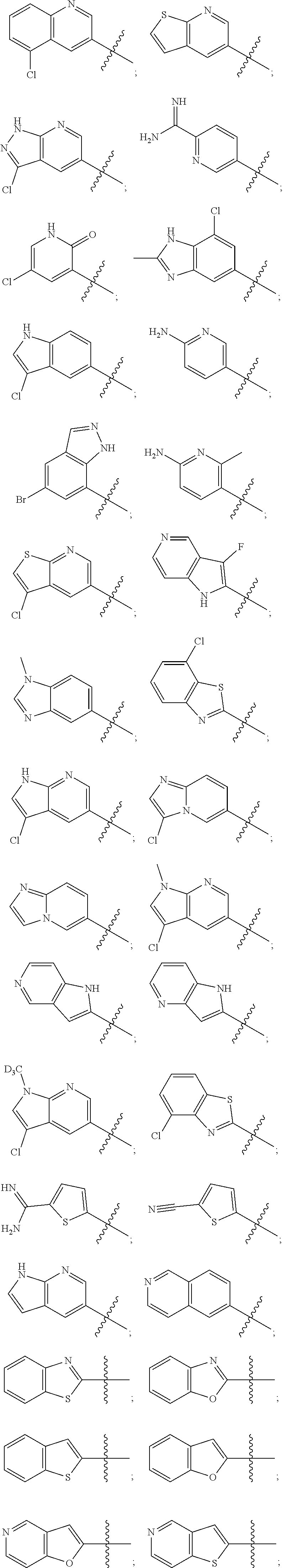

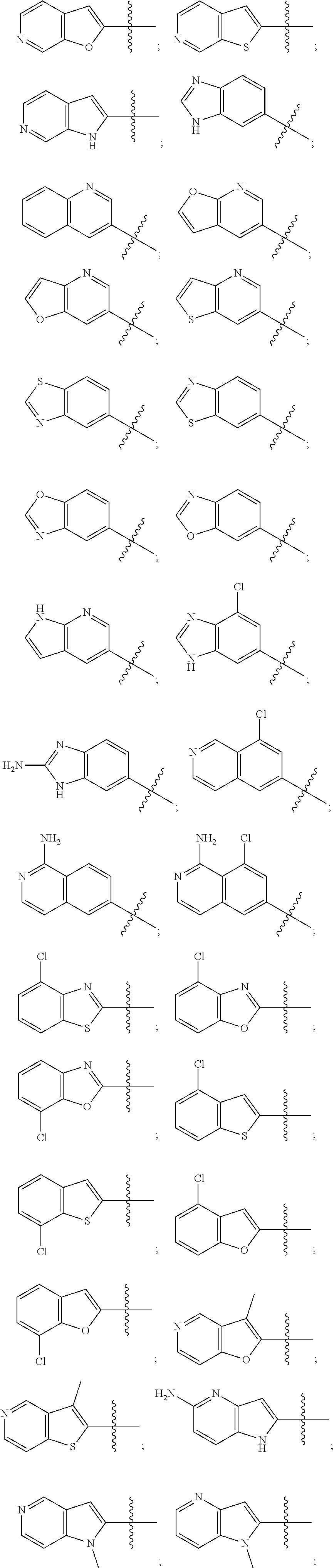

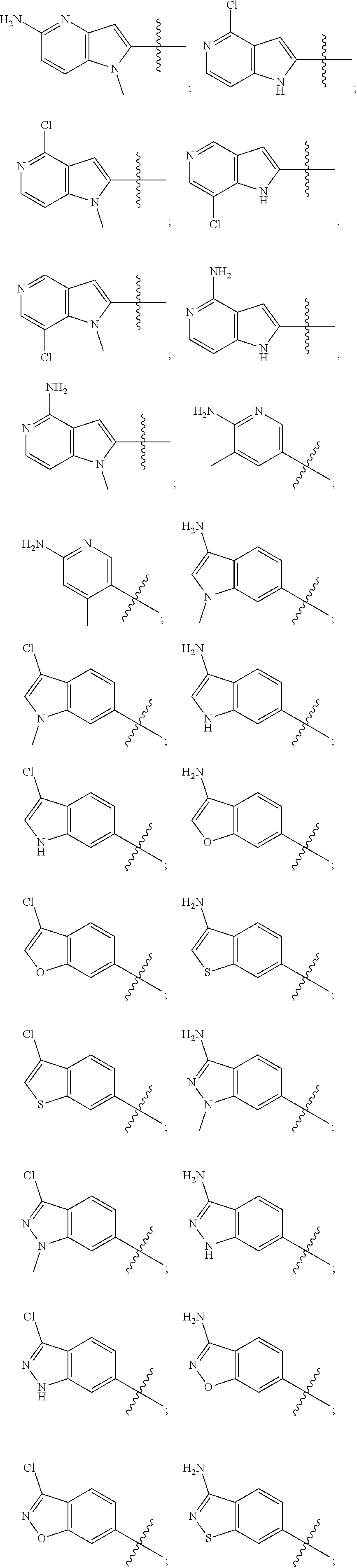

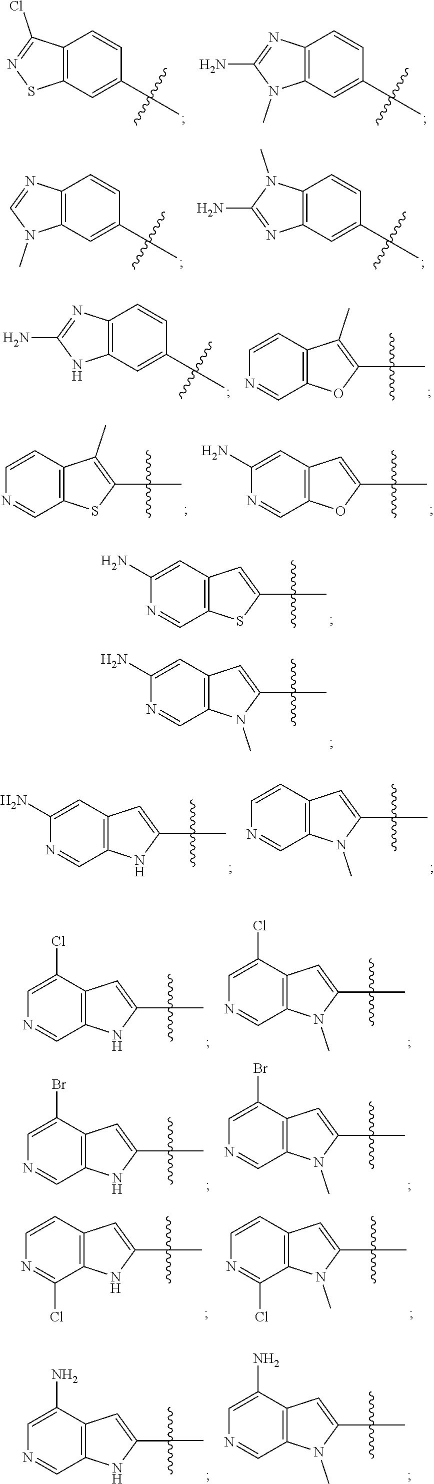

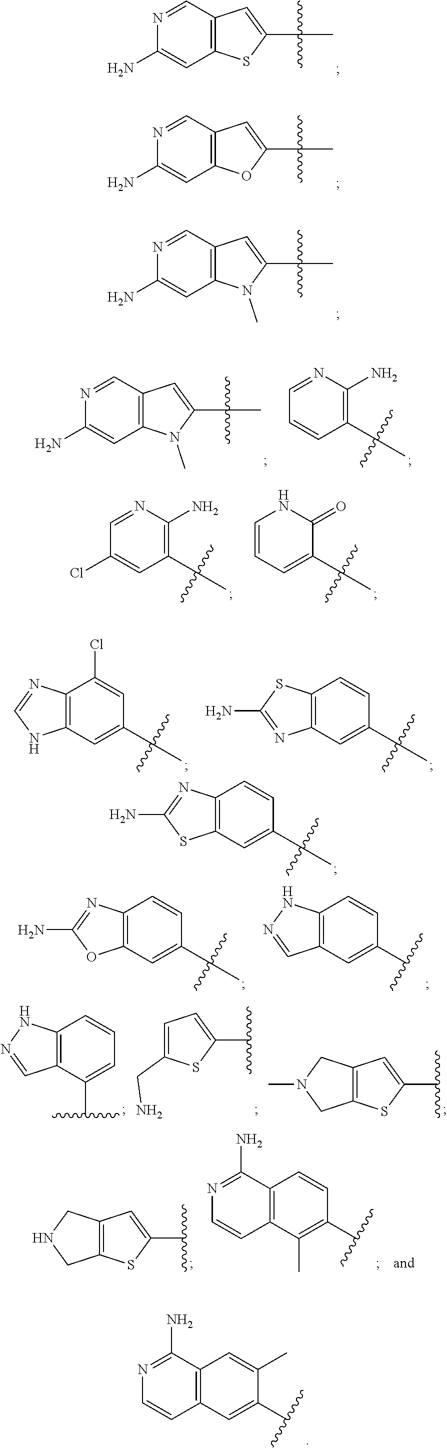

26. (canceled)

27. The compound of claim 9, wherein R¹ has one of the structures: 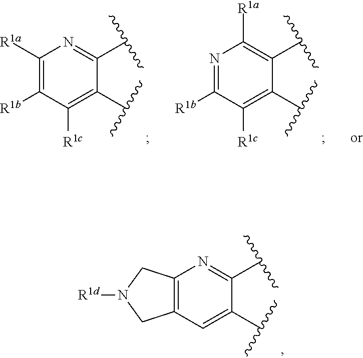
and
wherein R^{1a}, R^{1b}, and R^{1c} are each independently selected from the group consisting of hydrogen, C₁-C₆ alkyl, C₁-C₆ deuterated alkyl, amino, aminylalkyl, alkoxy, and halogen, and wherein R^{1d} is selected from the group consisting of hydrogen and C₁-C₆ alkyl.

28.-37. (canceled)

38. The compound of claim 1, wherein R⁷ is unsubstituted or substituted C₆-C₁₀ aryl, and the substituted C₆-C₁₀ aryl is substituted with one or more R^{7a}, R^{7b}, R^{7c}, R^{7d}, and R^{7e}, wherein the R^{7a}, R^{7b}, R^{7c}, R^{7d}, and R^{7e} are each independently selected from the group consisting of hydrogen, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃-C₁₀ cycloalkyl, and substituted or unsubstituted 4-10 membered heterocyclyl, or wherein any two of R^{7a}, R^{7b}, R^{7c}, R^{7d}, and R^{7e} are connected to the phenyl at adjacent carbon atoms, and the two R^{7a}, R^{7b}, R^{7c}, R^{7d}, and R^{7e} are connected, together with the carbon atoms to which they are attached, to form a substituted or unsubstituted C₃-C₆ cycloalkyl, or a substituted or unsubstituted 5- or 6-membered heterocyclic ring comprising 1-4 heteroatoms selected from the group consisting of N, S, and O, fused to the phenyl,
wherein R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, C₃-C₁₀ cycloalkyl, heterocyclyl, heteroarylalkyl, and heteroaryl,
wherein R^{7a}, R^{7b}, R^{7c}, R^{7d}, and R^{7e} are each substituted with one or more substituents selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when R^{7a}, R^{7b}, R^{7c}, R^{7d}, or R^{7e} is a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃-C₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, C₃-C₁₀ cycloalkyl, heterocyclyl, and heteroaryl, and
wherein when one or more chiral center is present, each chiral center has (R) stereochemistry, (S) stereochemistry, or is a mixture thereof.

39.-41. (canceled)

42. The compound of claim 1, wherein R⁷ is unsubstituted or substituted 5-9-membered heteroaryl, and the substituted 5-9-membered heteroaryl is substituted with one or more of R^{7a}, R^{7b}, R^{7c}, R^{7d}, and R^{7e}, wherein the R^{7a}, R^{7b}, R^{7c}, R^{7d}, and R^{7e} are each independently selected from the group consisting of hydrogen, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃-C₁₀ cycloalkyl, and substituted or unsubstituted 4-10 membered heterocyclyl, or wherein any two of R^{7a}, R^{7b}, R^{7c}, R^{7d}, and R^{7e} are connected to the phenyl at adjacent carbon atoms, and the two R^{7a}, R^{7b}, R^{7c}, R^{7d}, and R^{7e} are connected, together with the carbon atoms to which they are attached, to form a substituted or unsubstituted C₃-C₆ cycloalkyl, or a substituted or unsubstituted 5- or 6-membered heterocyclic ring comprising 1-4 heteroatoms selected from the group consisting of N, S, and O, fused to the phenyl,
wherein R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, C₃-C₁₀ cycloalkyl, heterocyclyl, heteroarylalkyl, and heteroaryl,
wherein R^{7a}, R^{7b}, R^{7c}, R^{7d}, and R^{7e} are each substituted with one or more substituents selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when R^{7a}, R^{7b}, R^{7c}, R^{7d}, or R^{7e} is a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃-C₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, C₃-C₁₀ cycloalkyl, heterocyclyl, and heteroaryl, and
wherein when one or more chiral center is present, each chiral center has (R) stereochemistry, (S) stereochemistry, or is a mixture thereof.

43.-47. (canceled)

48. The compound of claim 1, wherein L is —(CR^{8a}R^{8b})ₙ—, wherein each —(CR^{8a}R^{8b})— is the same or is different, and wherein n is 1, 2, or 3, or wherein R² and L, together with the nitrogen to which they are attached, form a 4-membered heterocyclyl.

49.-57. (canceled)

58. A compound of Formula (II): 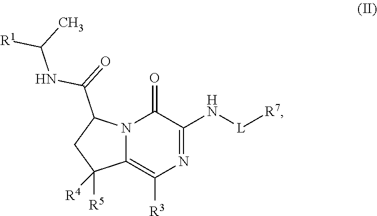
or a stereoisomer, tautomer, or pharmaceutically acceptable salt thereof, wherein:
R¹ is a substituted or unsubstituted 5-10-membered heteroaryl consisting of a 5-10-membered monocyclic or bicyclic ring system comprising one to eight carbon atoms, one to six heteroatoms selected from the group consisting of nitrogen, oxygen, and sulfur, and at least one aromatic ring;
R³ is selected from the group consisting of hydrogen, substituted or unsubstituted C₁-C₃ alkyl, substituted or unsubstituted C₃-C₆ cycloalkyl, substituted or unsubstituted phenyl, substituted or unsubstituted 5-6-membered heteroaryl, halogen, cyano, and CD₃, wherein the substituted C₁-C₃ alkyl, C₃-C₆ cycloalkyl, phenyl, and 5-6-membered heteroaryl of R³ are substituted with methyl, fluoroalkyl, difluoroalkyl, or trifluoroalkyl;
R⁴ is selected from the group consisting of hydrogen, C₁-C₆ alkyl, and benzyl, and R⁵ is hydrogen or methyl; or R⁴ and R⁵, together with the carbon to which they are attached, form a substituted or unsubstituted C₃-C₅ cycloalkyl or a 6-membered cycloalkenyl;
R⁷ is selected from the group consisting of hydrogen, substituted or unsubstituted C₁-C₃ alkyl, 6-membered heteroalkyl, substituted or unsubstituted C₃-C₆ cycloalkyl, C₃-C₆ cycloalkyl fused with substituted or unsubstituted C₆-C₁₀ aryl, substituted or unsubstituted 3-6-membered heterocyclyl, substituted or unsubstituted phenyl, substituted or unsubstituted phenyl fused with C₅-C₆ cycloalkyl, substituted or unsubstituted 5-9-membered heteroaryl, substituted or unsubstituted 5-9-membered heteroaryl fused with C₅-C₆ cycloalkyl, methoxy, and halogen, and when R⁷ is hydrogen, n is 0;
L is —(CR^{8a}R^{8b})ₙ—, wherein each —(CR^{8a}R^{8b})— is the same or is different, wherein R^{8a} and R^{8b} are each independently selected from the group consisting of hydrogen, C₁-C₄ linear or branched alkyl, hydroxyalkyl, and alkoxy, or R^{8a} and R^{8b} together with the carbon to which they are attached form a C₃-C₆ cycloalkyl or a 4-membered heterocyclyl; and
n is 0, 1, 2, 3, or 4, and
wherein when one or more chiral center is present, each chiral center has (R) stereochemistry, (S) stereochemistry, or is a mixture thereof.

59.-73. (canceled)

74. A compound of Formula (III): 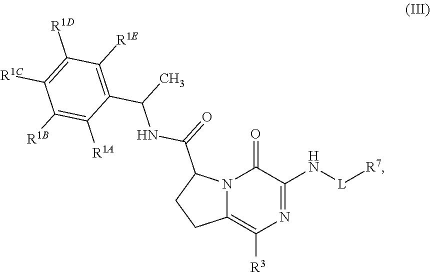
wherein:
R^{1A}, R^{1B}, R^{1C}, R^{1D}, and R^{1E} are each independently selected from the group consisting of hydrogen, linear or branched C₁-C₃ alkyl, linear or branched C₁-C₃ aminylalkyl, halogen, tetrazolyl, and amidinyl, and wherein at least one of R^{1A}, R^{1B}, R^{1C}, R^{1D}, and R^{1E} is not H;
R³ is hydrogen or halogen;
R⁷ is a substituted or unsubstituted C₆-C₁₀ aryl, wherein the substituted C₆-C₁₀ aryl is substituted with one or more halogen, one or more C₁-C₃ alkyl, or a combination thereof; and
L is —CH₂— or —CH₂CH₂—, and
wherein when one or more chiral center is present, each chiral center has (R) stereochemistry, (S) stereochemistry, or is a mixture thereof

75.-77. (canceled)

78. A compound of Formula (IV): 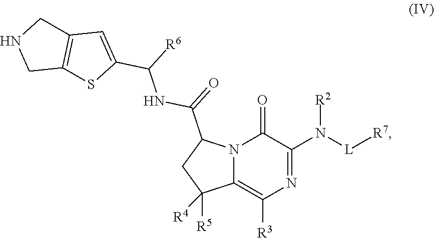
or a stereoisomer, tautomer, or pharmaceutically acceptable salt thereof, wherein:
R² is hydrogen, or R² and L, together with the nitrogen to which they are attached, form a substituted or unsubstituted 3-6-membered heterocyclyl;
L is —(CR^{8a}R^{8b})ₙ—, wherein each —(CR^{8a}R^{8b})— is the same or is different;
R³ is selected from the group consisting of hydrogen, substituted or unsubstituted C₁-C₃ alkyl, substituted or unsubstituted C₃-C₆ cycloalkyl, substituted or unsubstituted 5-6-membered saturated or unsaturated heterocyclyl, substituted or unsubstituted phenyl, substituted or unsubstituted 5-6-membered heteroaryl, halogen, and cyano;
R⁴ and R⁵ are each independently selected from the group consisting of hydrogen, substituted or unsubstituted C₁-C₆ alkyl, and substituted or unsubstituted benzyl, or R⁴ and R⁵, together with the carbon to which they are attached, form a substituted or unsubstituted C₃-C₆ cycloalkyl or a substituted or unsubstituted 5-6-membered cycloalkenyl;
R⁶ is selected from the group consisting of hydrogen and methyl;
R⁷ is selected from the group consisting of hydrogen, substituted or unsubstituted C₁-C₃ alkyl, substituted or unsubstituted 5-6-membered heteroalkyl, substituted or unsubstituted C₃-C₆ cycloalkyl, cycloalkyl fused with substituted or unsubstituted phenyl, substituted or unsubstituted 3-6-membered heterocyclyl, substituted or unsubstituted C₆-C₁₀ aryl, substituted or unsubstituted C₆-C₁₀ aryl fused with C₅-C₆ cycloalkyl, substituted or unsubstituted 5-9-membered heteroaryl, substituted or unsubstituted 5-9-membered heteroaryl fused with C₅-C₆ cycloalkyl, alkoxy, and halogen, wherein when R⁷ is hydrogen, n is 0;
R^{8a} and R^{8b} are each independently selected from the group consisting of hydrogen, C₁-C₄ linear alkyl, C₃-C₄ branched alkyl, C₃-C₅ cycloalkyl, hydroxyalkyl, and alkoxy, or R^{8a} and R^{8b} together, with the carbon to which they are attached, form a substituted or unsubstituted C₃-C₆ cycloalkyl or a substituted or unsubstituted 3-6-membered heterocyclyl; and
n is 0, 1, 2, 3, or 4, and
wherein when one or more chiral center is present, each chiral center has (R) stereochemistry, (S) stereochemistry, or is a mixture thereof.

79.-92. (canceled)

93. A compound of Formula (V): 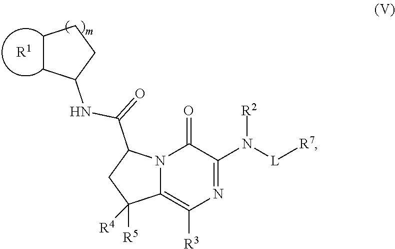
or a stereoisomer, tautomer, or pharmaceutically acceptable salt thereof, wherein:
R¹ is a substituted or unsubstituted aryl, or a substituted or unsubstituted 5-10-membered heteroaryl;
R² is hydrogen, or R² and L, together with the nitrogen to which they are attached, form a substituted or unsubstituted 3-6-membered heterocyclyl;
R³ is selected from the group consisting of hydrogen, substituted or unsubstituted C₁-C₃ alkyl, substituted or unsubstituted C₃-C₆ cycloalkyl, substituted or unsubstituted phenyl, substituted or unsubstituted 5-6-membered heteroaryl, halogen, cyano, and CD₃, wherein the substituted C₁-C₃ alkyl, C₃-C₆ cycloalkyl, phenyl, and 5-6-membered heteroaryl of R³ are substituted with methyl, fluoroalkyl, difluoroalkyl, or trifluoroalkyl;
R⁴ is selected from the group consisting of hydrogen, C₁-C₆ alkyl, and benzyl, and R⁵ is hydrogen or methyl; or R⁴ and R⁵, together with the carbon to which they are attached, form a C₃-C₆ cycloalkyl or a 6-membered cycloalkenyl;
R⁷ is selected from the group consisting of hydrogen, substituted or unsubstituted C₁-C₃ alkyl, 6-membered heteroalkyl, substituted or unsubstituted C₃-C₆ cycloalkyl, substituted or unsubstituted C₃-C₆ cycloalkyl fused with C₆-C₁₀ aryl, substituted or unsubstituted 3-6-membered heterocyclyl, substituted or unsubstituted phenyl, substituted or unsubstituted phenyl fused with C₅-C₆ cycloalkyl, substituted or unsubstituted 5-9-membered heteroaryl, substituted or unsubstituted 5-9-membered heteroaryl fused with C₅-C₆ cycloalkyl, methoxy, and halogen, and wherein when R⁷ is hydrogen, n is 0;
L is —(CR^{8a}R^{8b})ₙ—, wherein each —(CR^{8a}R^{8b})— is the same or is different, wherein R^{8a} and R^{8b} are each independently selected from the group consisting of hydrogen, C₁-C₄ linear or branched alkyl, hydroxyalkyl, and alkoxy, or R^{8a} and R^{8b} together with the carbon to which they are attached form a C₃-C₆ cycloalkyl or a 4-membered heterocyclyl; and
n is 0, 1, 2, 3, or 4, and
wherein when one or more chiral center is present, each chiral center has (R) stereochemistry, (S) stereochemistry, or is a mixture thereof.

94.-106. (canceled)

107. The compound of claim 1, wherein the salt of the pharmaceutically acceptable salt is selected from the group consisting of trifluoroacetic acid, hydrogen chloride, acetic acid, hydrogen bromide, sulfuric acid, phosphoric acid, maleic acid, fumaric acid, lactic acid, tartaric acid, citric acid, gluconic acid, and a combination thereof.

108. (canceled)

109. The compound of claim 1, having a structure selected from the group consisting of: 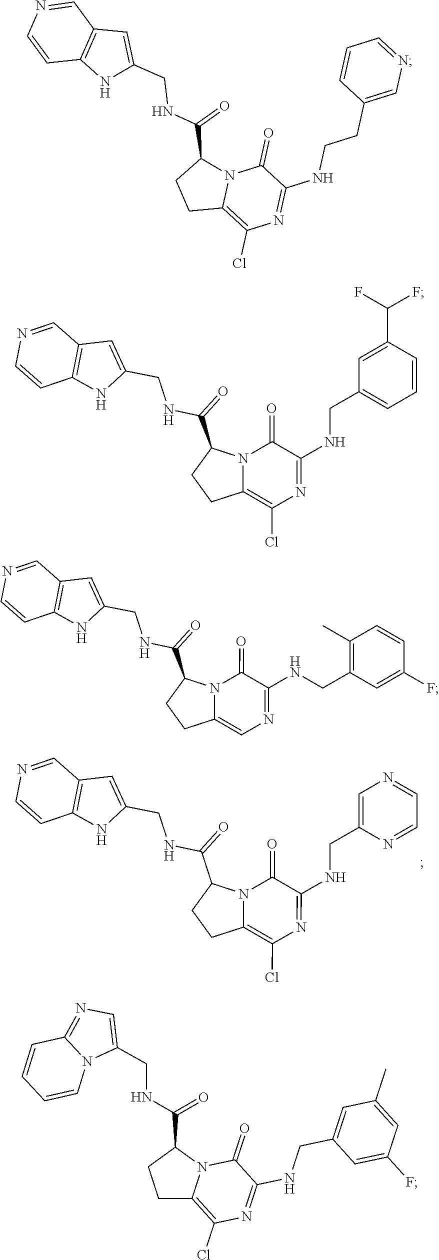 [SMILES: O=C(NCc1cc2cnccc2[nH]1)[C@@H]1CCc2c(Cl)nc(NCCc3cccnc3)c(=O)n21 ; O=C(NCc1cc2cnccc2[nH]1)[C@@H]1CCc2c(Cl)nc(NCc3cccc(C(F)F)c3)c(=O)n21 ; Cc1ccc(F)cc1CNc1ncc2n(c1=O)[C@H](C(=O)NCc1cc3cnccc3[nH]1)CC2 ; O=C(NCc1cc2cnccc2[nH]1)C1CCc2c(Cl)nc(NCc3cnccn3)c(=O)n21 ; Cc1cc(F)cc(CNc2nc(Cl)c3n(c2=O)[C@H](C(=O)NCc2cnc4ccccn24)CC3)c1] 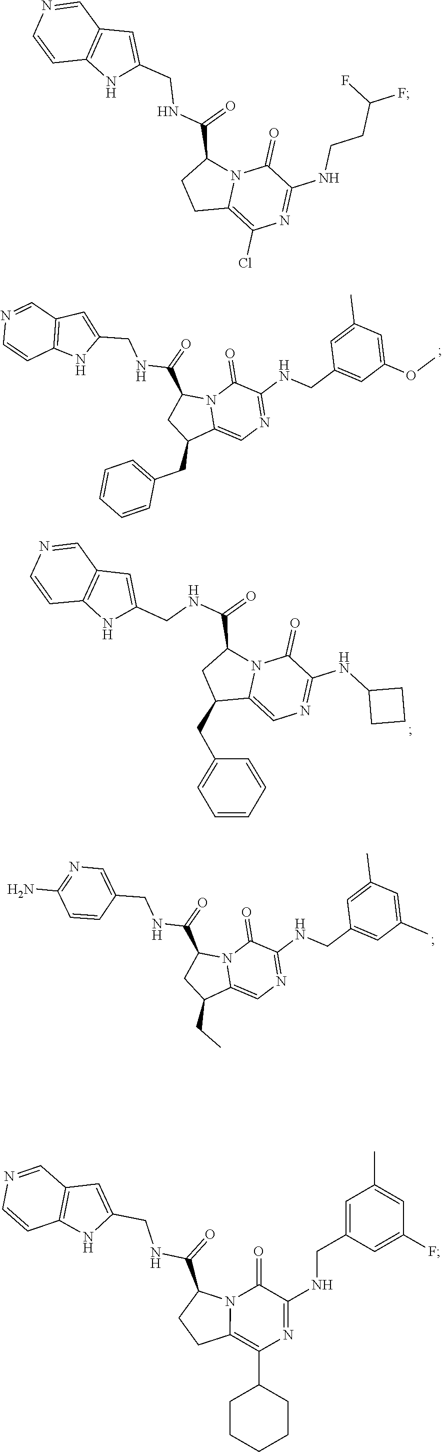 [SMILES: O=C(NCc1cc2cnccc2[nH]1)[C@@H]1CCc2c(Cl)nc(NCCC(F)F)c(=O)n21 ; COc1cc(C)cc(CNc2ncc3n(c2=O)[C@H](C(=O)NCc2cc4cnccc4[nH]2)C[C@@H]3Cc2ccccc2)c1 ; O=C(NCc1cc2cnccc2[nH]1)[C@@H]1C[C@H](Cc2ccccc2)c2cnc(NC3CCC3)c(=O)n21 ; CC[C@H]1C[C@@H](C(=O)NCc2ccc(N)nc2)n2c1cnc(NCc1cc(C)cc(C)c1)c2=O ; Cc1cc(F)cc(CNc2nc(C3CCCCC3)c3n(c2=O)[C@H](C(=O)NCc2cc4cnccc4[nH]2)CC3)c1] 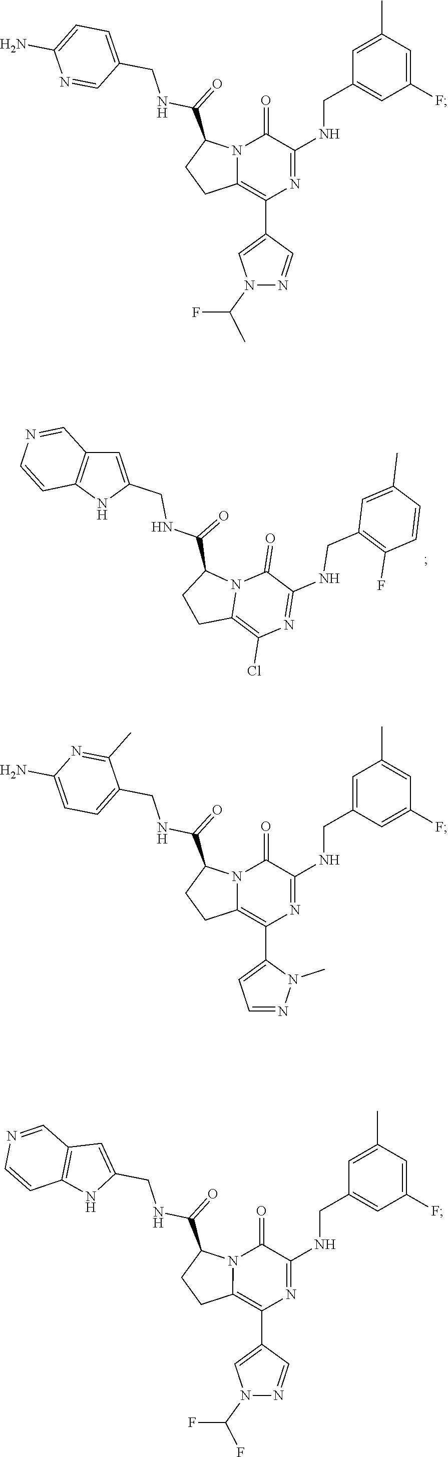 [SMILES: Cc1cc(F)cc(CNc2nc(-c3cnn(C(C)F)c3)c3n(c2=O)[C@H](C(=O)NCc2ccc(N)nc2)CC3)c1 ; Cc1ccc(F)c(CNc2nc(Cl)c3n(c2=O)[C@H](C(=O)NCc2cc4cnccc4[nH]2)CC3)c1 ; Cc1cc(F)cc(CNc2nc(-c3ccnn3C)c3n(c2=O)[C@H](C(=O)NCc2ccc(N)nc2C)CC3)c1 ; Cc1cc(F)cc(CNc2nc(-c3cnn(C(F)F)c3)c3n(c2=O)[C@H](C(=O)NCc2cc4cnccc4[nH]2)CC3)c1] 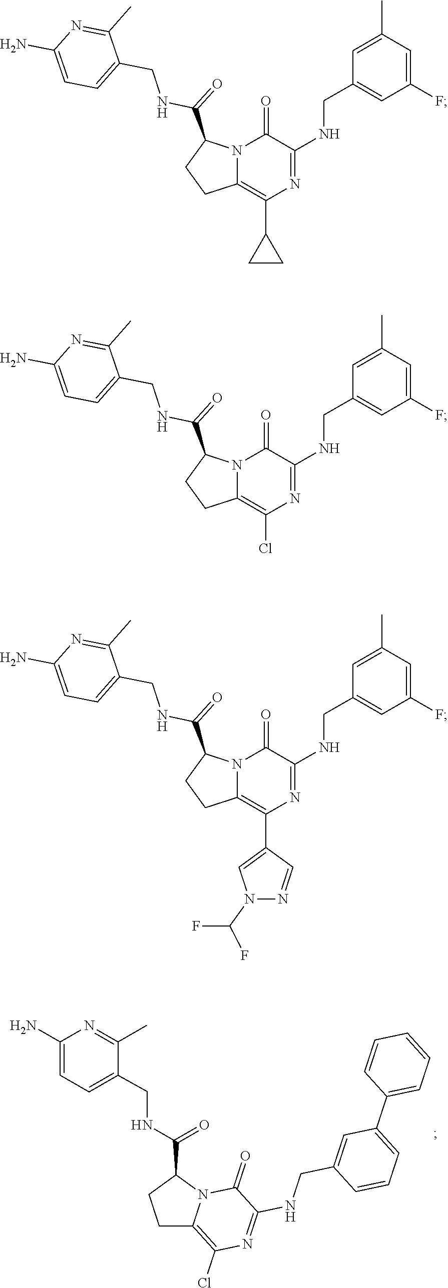 [SMILES: Cc1cc(F)cc(CNc2nc(C3CC3)c3n(c2=O)[C@H](C(=O)NCc2ccc(N)nc2C)CC3)c1 ; Cc1cc(F)cc(CNc2nc(Cl)c3n(c2=O)[C@H](C(=O)NCc2ccc(N)nc2C)CC3)c1 ; Cc1cc(F)cc(CNc2nc(-c3cnn(C(F)F)c3)c3n(c2=O)[C@H](C(=O)NCc2ccc(N)nc2C)CC3)c1 ; Cc1nc(N)ccc1CNC(=O)[C@@H]1CCc2c(Cl)nc(NCc3cccc(-c4ccccc4)c3)c(=O)n21] 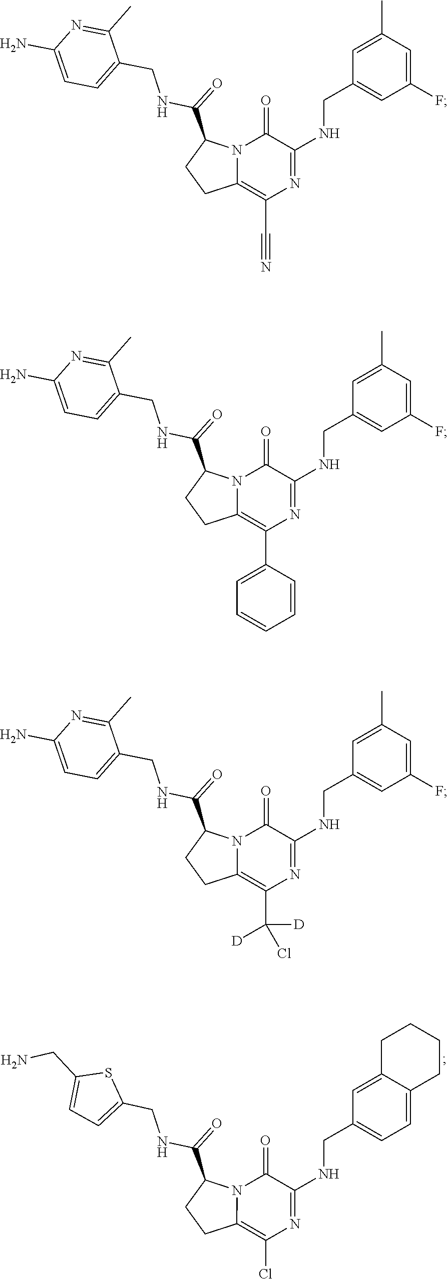 [SMILES: Cc1cc(F)cc(CNc2nc(C#N)c3n(c2=O)[C@H](C(=O)NCc2ccc(N)nc2C)CC3)c1 ; Cc1cc(F)cc(CNc2nc(-c3ccccc3)c3n(c2=O)[C@H](C(=O)NCc2ccc(N)nc2C)CC3)c1 ; [2H]C([2H])(Cl)c1nc(NCc2cc(C)cc(F)c2)c(=O)n2c1CC[C@H]2C(=O)NCc1ccc(N)nc1C ; NCc1ccc(CNC(=O)[C@@H]2CCc3c(Cl)nc(NCc4ccc5c(c4)CCCC5)c(=O)n32)s1] 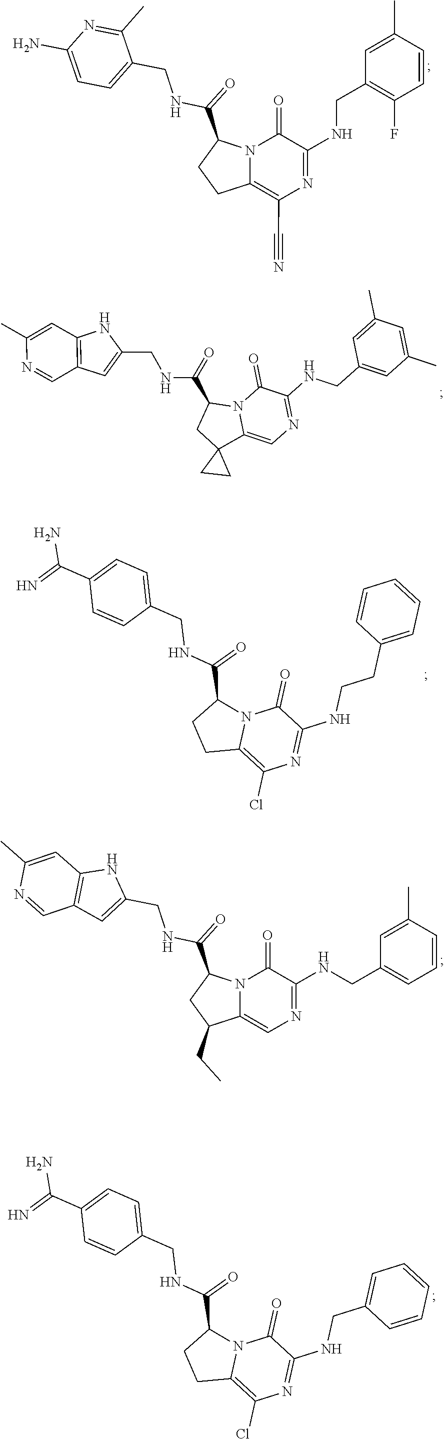 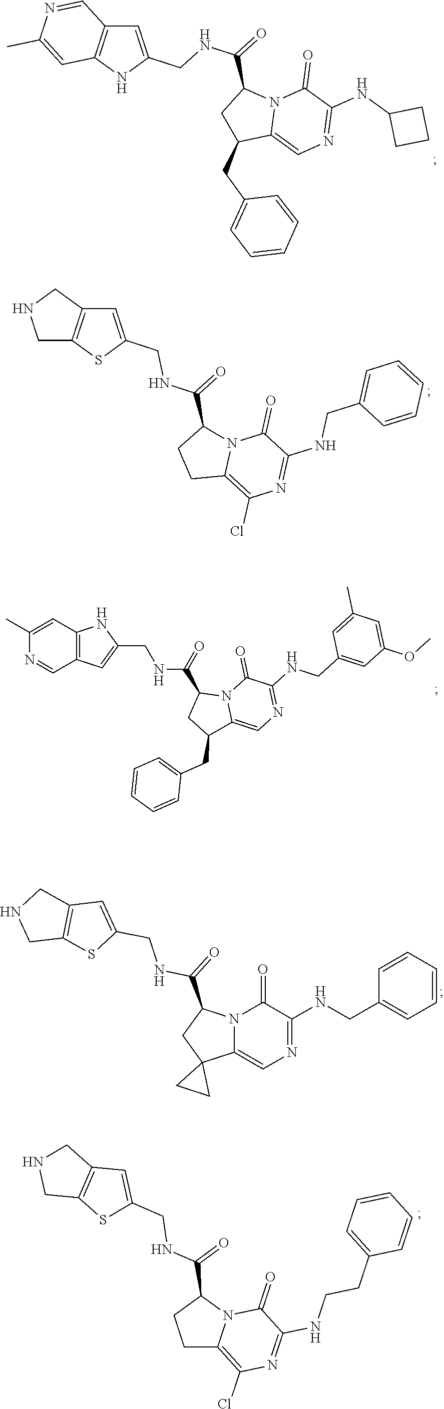 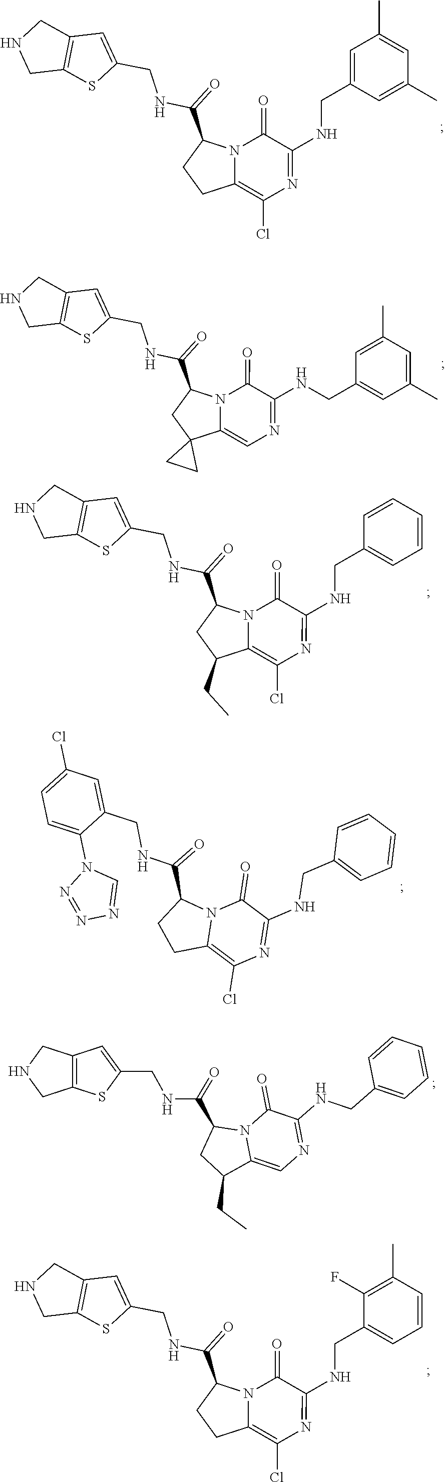 [SMILES: Cc1cc(C)cc(CNc2nc(Cl)c3n(c2=O)[C@H](C(=O)NCc2cc4c(s2)CNC4)CC3)c1 ; Cc1cc(C)cc(CNc2ncc3n(c2=O)[C@H](C(=O)NCc2cc4c(s2)CNC4)CC32CC2)c1 ; CC[C@H]1C[C@@H](C(=O)NCc2cc3c(s2)CNC3)n2c1c(Cl)nc(NCc1ccccc1)c2=O ; O=C(NCc1cc(Cl)ccc1-n1cnnn1)[C@@H]1CCc2c(Cl)nc(NCc3ccccc3)c(=O)n21 ; CC[C@H]1C[C@@H](C(=O)NCc2cc3c(s2)CNC3)n2c1cnc(NCc1ccccc1)c2=O ; Cc1cccc(CNc2nc(Cl)c3n(c2=O)[C@H](C(=O)NCc2cc4c(s2)CNC4)CC3)c1F] 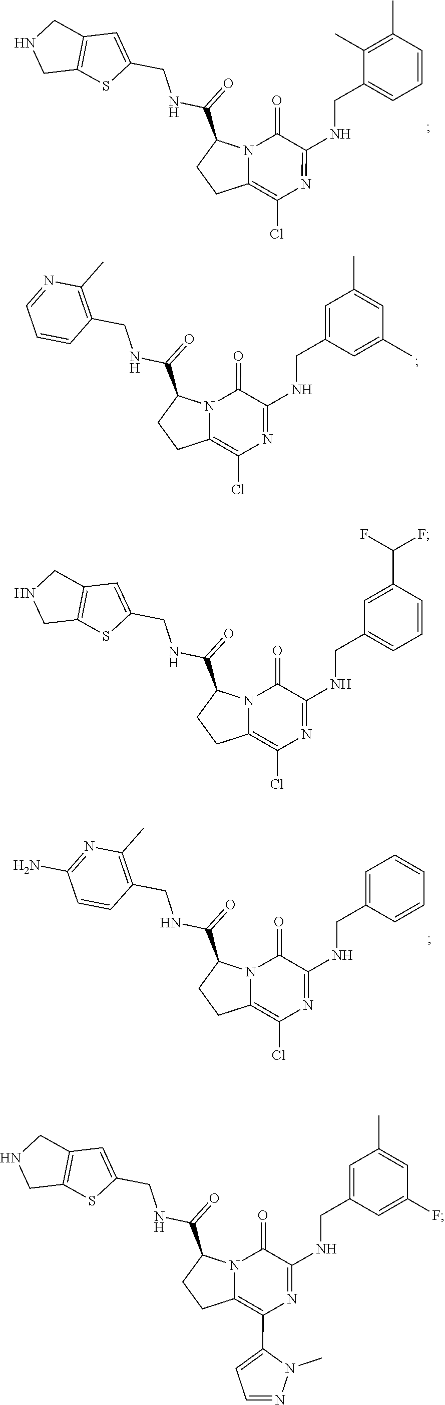 [SMILES: Cc1cccc(CNc2nc(Cl)c3n(c2=O)[C@H](C(=O)NCc2cc4c(s2)CNC4)CC3)c1C ; Cc1cc(C)cc(CNc2nc(Cl)c3n(c2=O)[C@H](C(=O)NCc2cccnc2C)CC3)c1 ; O=C(NCc1cc2c(s1)CNC2)[C@@H]1CCc2c(Cl)nc(NCc3cccc(C(F)F)c3)c(=O)n21 ; Cc1nc(N)ccc1CNC(=O)[C@@H]1CCc2c(Cl)nc(NCc3ccccc3)c(=O)n21 ; Cc1cc(F)cc(CNc2nc(-c3ccnn3C)c3n(c2=O)[C@H](C(=O)NCc2cc4c(s2)CNC4)CC3)c1] 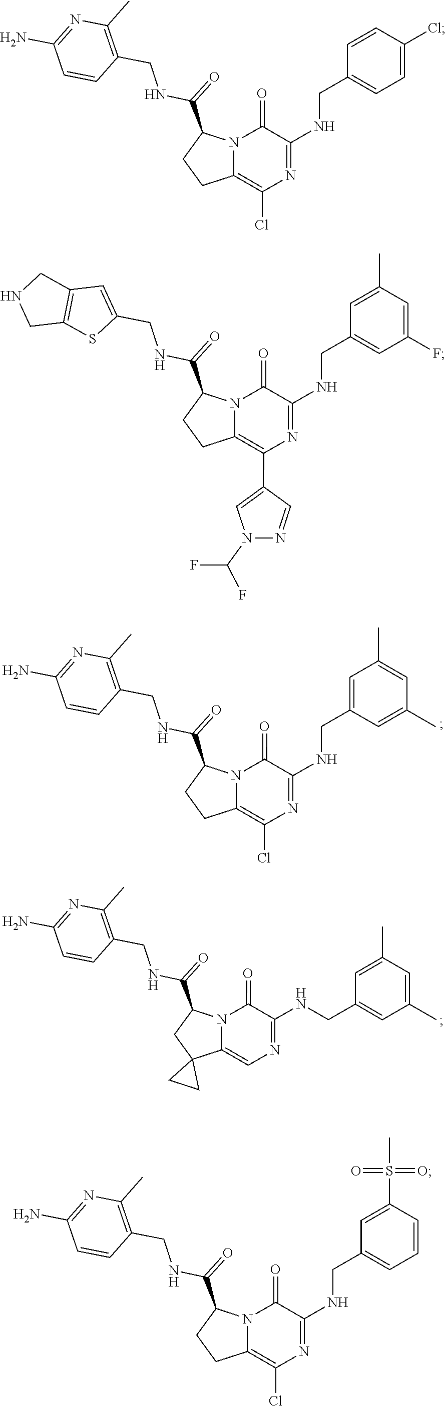 [SMILES: Cc1nc(N)ccc1CNC(=O)[C@@H]1CCc2c(Cl)nc(NCc3ccc(Cl)cc3)c(=O)n21 ; Cc1cc(F)cc(CNc2nc(-c3cnn(C(F)F)c3)c3n(c2=O)[C@H](C(=O)NCc2cc4c(s2)CNC4)CC3)c1 ; Cc1cc(C)cc(CNc2nc(Cl)c3n(c2=O)[C@H](C(=O)NCc2ccc(N)nc2C)CC3)c1 ; Cc1cc(C)cc(CNc2ncc3n(c2=O)[C@H](C(=O)NCc2ccc(N)nc2C)CC32CC2)c1 ; Cc1nc(N)ccc1CNC(=O)[C@@H]1CCc2c(Cl)nc(NCc3cccc(S(C)(=O)=O)c3)c(=O)n21] 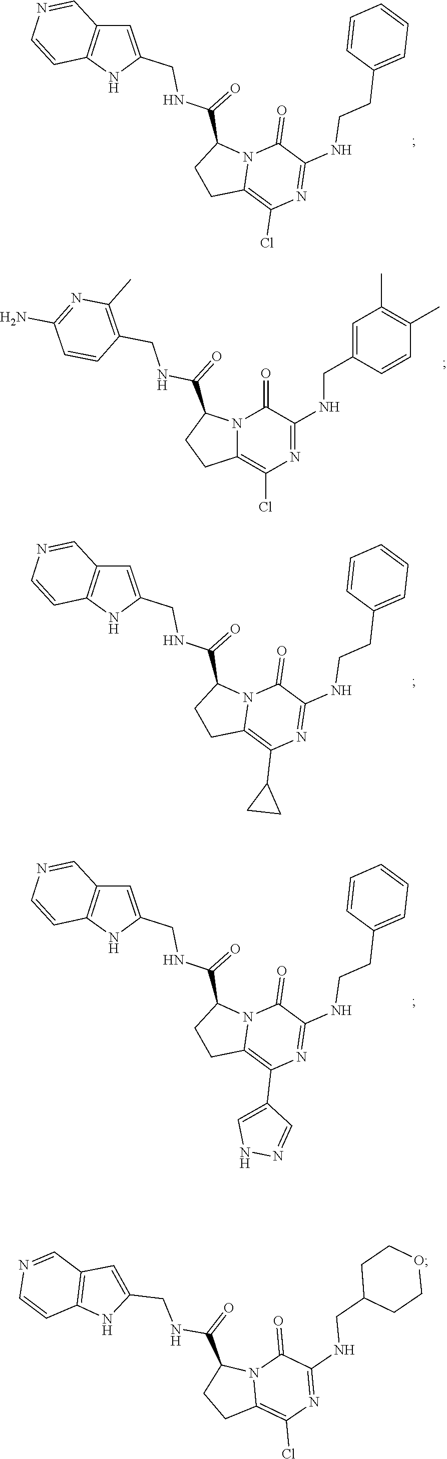 [SMILES: O=C(NCc1cc2cnccc2[nH]1)[C@@H]1CCc2c(Cl)nc(NCCc3ccccc3)c(=O)n21 ; Cc1ccc(CNc2nc(Cl)c3n(c2=O)[C@H](C(=O)NCc2ccc(N)nc2C)CC3)cc1C ; O=C(NCc1cc2cnccc2[nH]1)[C@@H]1CCc2c(C3CC3)nc(NCCc3ccccc3)c(=O)n21 ; O=C(NCc1cc2cnccc2[nH]1)[C@@H]1CCc2c(-c3cn[nH]c3)nc(NCCc3ccccc3)c(=O)n21 ; O=C(NCc1cc2cnccc2[nH]1)[C@@H]1CCc2c(Cl)nc(NCC3CCOCC3)c(=O)n21] 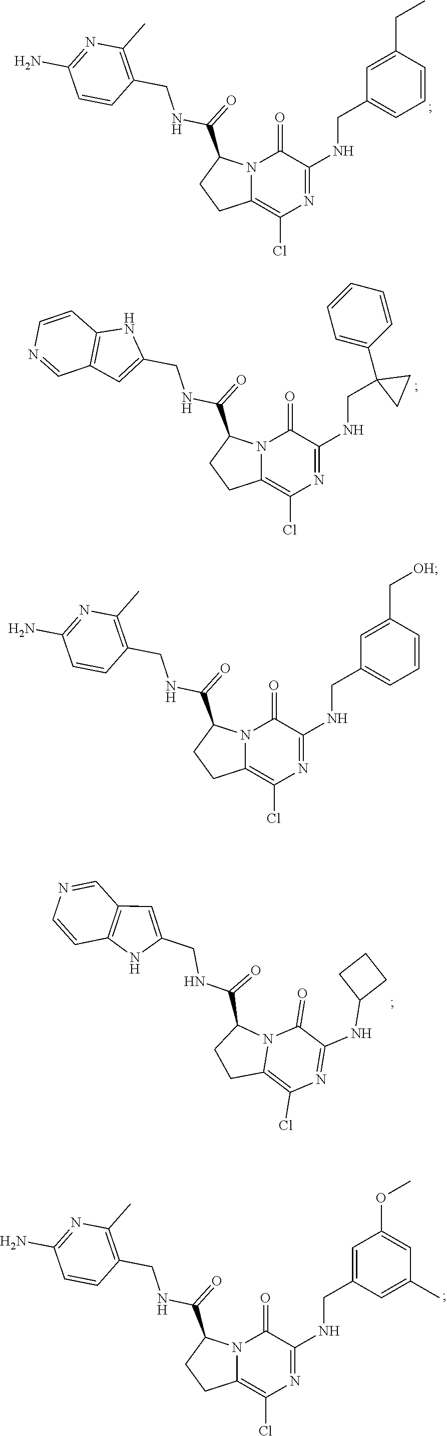 [SMILES: CCc1cccc(CNc2nc(Cl)c3n(c2=O)[C@H](C(=O)NCc2ccc(N)nc2C)CC3)c1 ; O=C(NCc1cc2cnccc2[nH]1)[C@@H]1CCc2c(Cl)nc(NCC3(c4ccccc4)CC3)c(=O)n21 ; Cc1nc(N)ccc1CNC(=O)[C@@H]1CCc2c(Cl)nc(NCc3cccc(CO)c3)c(=O)n21 ; O=C(NCc1cc2cnccc2[nH]1)[C@@H]1CCc2c(Cl)nc(NC3CCC3)c(=O)n21 ; COc1cc(C)cc(CNc2nc(Cl)c3n(c2=O)[C@H](C(=O)NCc2ccc(N)nc2C)CC3)c1] 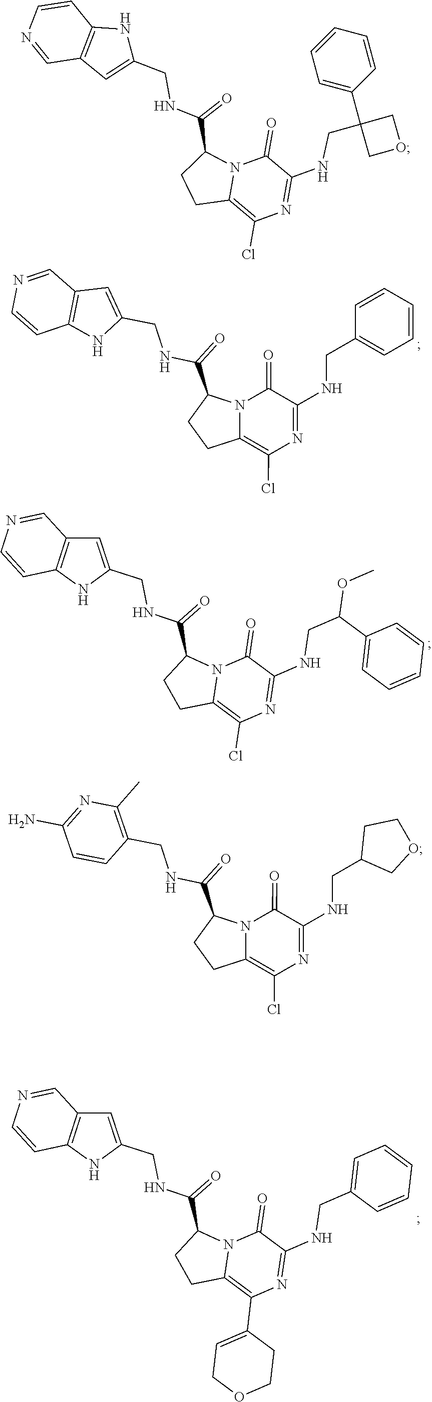 [SMILES: O=C(NCc1cc2cnccc2[nH]1)[C@@H]1CCc2c(Cl)nc(NCC3(c4ccccc4)COC3)c(=O)n21 ; O=C(NCc1cc2cnccc2[nH]1)[C@@H]1CCc2c(Cl)nc(NCc3ccccc3)c(=O)n21 ; COC(CNc1nc(Cl)c2n(c1=O)[C@H](C(=O)NCc1cc3cnccc3[nH]1)CC2)c1ccccc1 ; Cc1nc(N)ccc1CNC(=O)[C@@H]1CCc2c(Cl)nc(NCC3CCOC3)c(=O)n21 ; O=C(NCc1cc2cnccc2[nH]1)[C@@H]1CCc2c(C3=CCOCC3)nc(NCc3ccccc3)c(=O)n21] 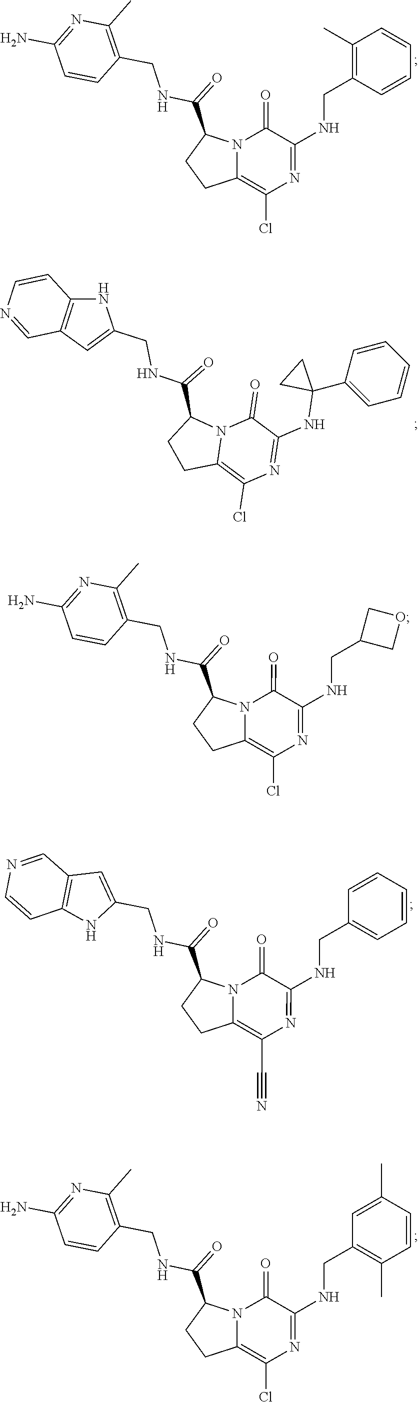 [SMILES: Cc1ccccc1CNc1nc(Cl)c2n(c1=O)[C@H](C(=O)NCc1ccc(N)nc1C)CC2 ; O=C(NCc1cc2cnccc2[nH]1)[C@@H]1CCc2c(Cl)nc(NC3(c4ccccc4)CC3)c(=O)n21 ; Cc1nc(N)ccc1CNC(=O)[C@@H]1CCc2c(Cl)nc(NCC3COC3)c(=O)n21 ; N#Cc1nc(NCc2ccccc2)c(=O)n2c1CC[C@H]2C(=O)NCc1cc2cnccc2[nH]1 ; Cc1ccc(C)c(CNc2nc(Cl)c3n(c2=O)[C@H](C(=O)NCc2ccc(N)nc2C)CC3)c1] 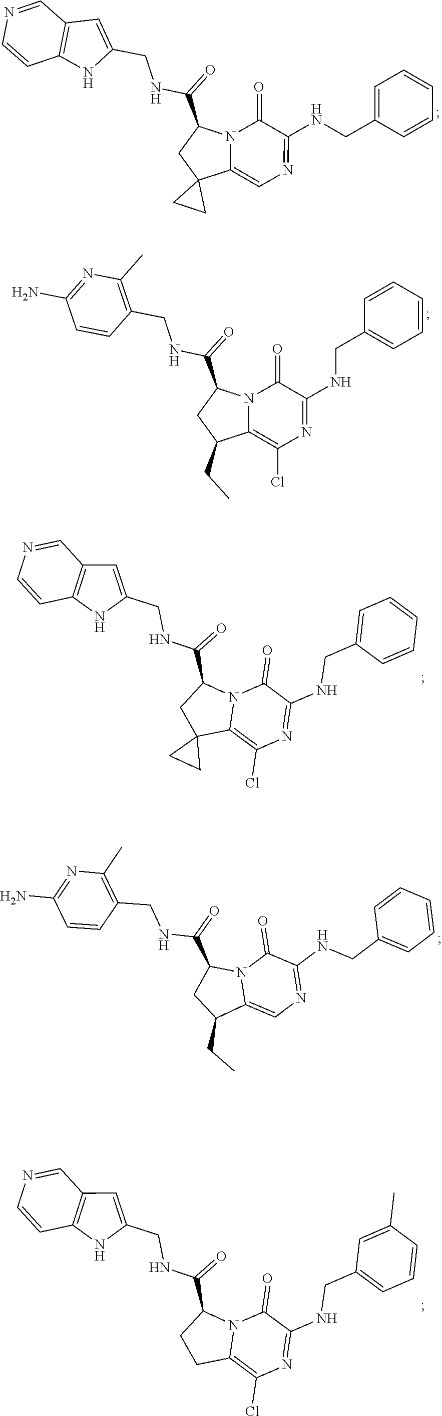 [SMILES: O=C(NCc1cc2cnccc2[nH]1)[C@@H]1CC2(CC2)c2cnc(NCc3ccccc3)c(=O)n21 ; CC[C@H]1C[C@@H](C(=O)NCc2ccc(N)nc2C)n2c1c(Cl)nc(NCc1ccccc1)c2=O ; O=C(NCc1cc2cnccc2[nH]1)[C@@H]1CC2(CC2)c2c(Cl)nc(NCc3ccccc3)c(=O)n21 ; CC[C@H]1C[C@@H](C(=O)NCc2ccc(N)nc2C)n2c1cnc(NCc1ccccc1)c2=O ; Cc1cccc(CNc2nc(Cl)c3n(c2=O)[C@H](C(=O)NCc2cc4cnccc4[nH]2)CC3)c1] 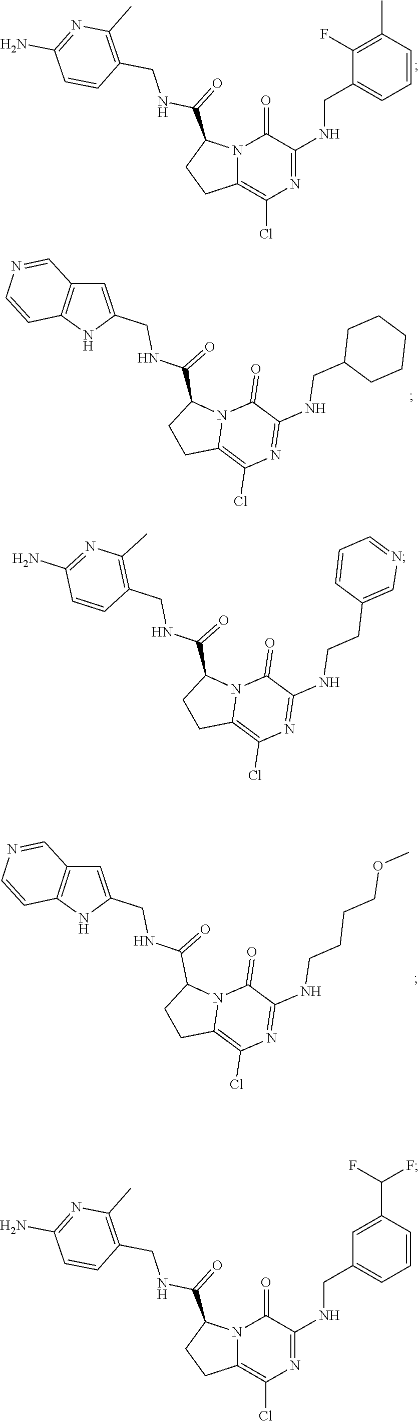 [SMILES: Cc1cccc(CNc2nc(Cl)c3n(c2=O)[C@H](C(=O)NCc2ccc(N)nc2C)CC3)c1F ; O=C(NCc1cc2cnccc2[nH]1)[C@@H]1CCc2c(Cl)nc(NCC3CCCCC3)c(=O)n21 ; Cc1nc(N)ccc1CNC(=O)[C@@H]1CCc2c(Cl)nc(NCCc3cccnc3)c(=O)n21 ; COCCCCNc1nc(Cl)c2n(c1=O)C(C(=O)NCc1cc3cnccc3[nH]1)CC2 ; Cc1nc(N)ccc1CNC(=O)[C@@H]1CCc2c(Cl)nc(NCc3cccc(C(F)F)c3)c(=O)n21] 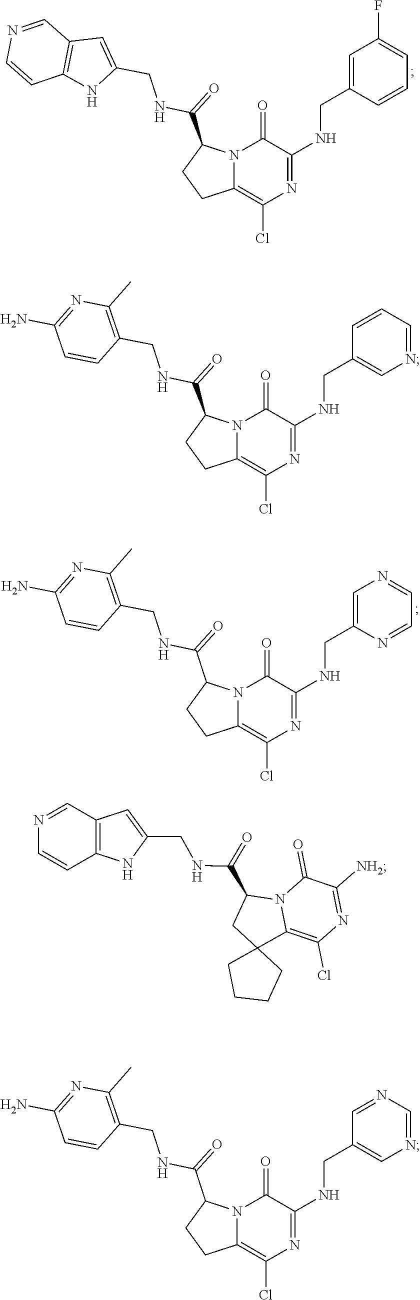 [SMILES: O=C(NCc1cc2cnccc2[nH]1)[C@@H]1CCc2c(Cl)nc(NCc3cccc(F)c3)c(=O)n21 ; Cc1nc(N)ccc1CNC(=O)[C@@H]1CCc2c(Cl)nc(NCc3cccnc3)c(=O)n21 ; Cc1nc(N)ccc1CNC(=O)C1CCc2c(Cl)nc(NCc3cnccn3)c(=O)n21 ; Nc1nc(Cl)c2n(c1=O)[C@H](C(=O)NCc1cc3cnccc3[nH]1)CC21CCCC1 ; Cc1nc(N)ccc1CNC(=O)C1CCc2c(Cl)nc(NCc3cncnc3)c(=O)n21] 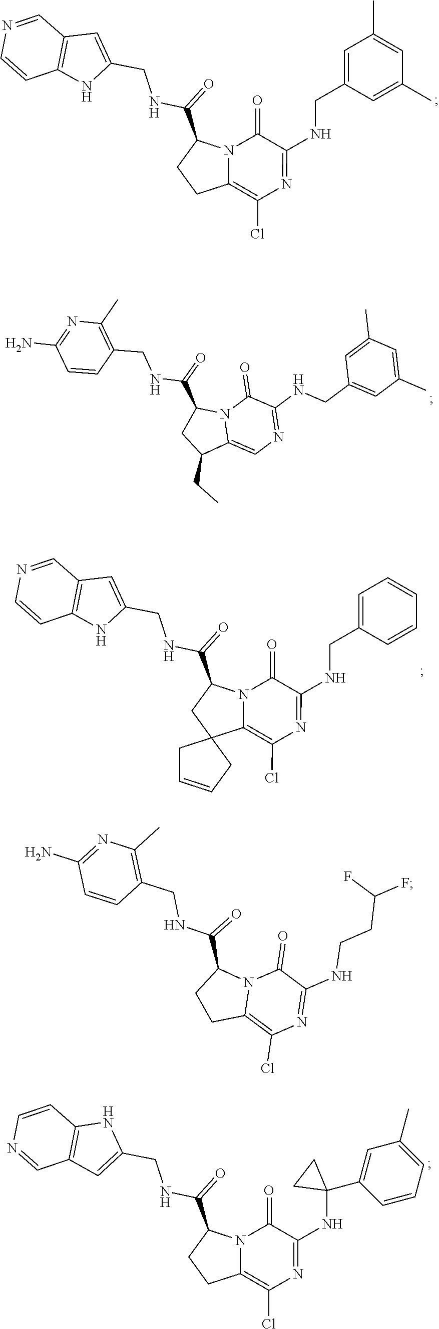 [SMILES: Cc1cc(C)cc(CNc2nc(Cl)c3n(c2=O)[C@H](C(=O)NCc2cc4cnccc4[nH]2)CC3)c1 ; CC[C@H]1C[C@@H](C(=O)NCc2ccc(N)nc2C)n2c1cnc(NCc1cc(C)cc(C)c1)c2=O ; O=C(NCc1cc2cnccc2[nH]1)[C@@H]1CC2(CC=CC2)c2c(Cl)nc(NCc3ccccc3)c(=O)n21 ; Cc1nc(N)ccc1CNC(=O)[C@@H]1CCc2c(Cl)nc(NCCC(F)F)c(=O)n21 ; Cc1cccc(C2(Nc3nc(Cl)c4n(c3=O)[C@H](C(=O)NCc3cc5cnccc5[nH]3)CC4)CC2)c1] 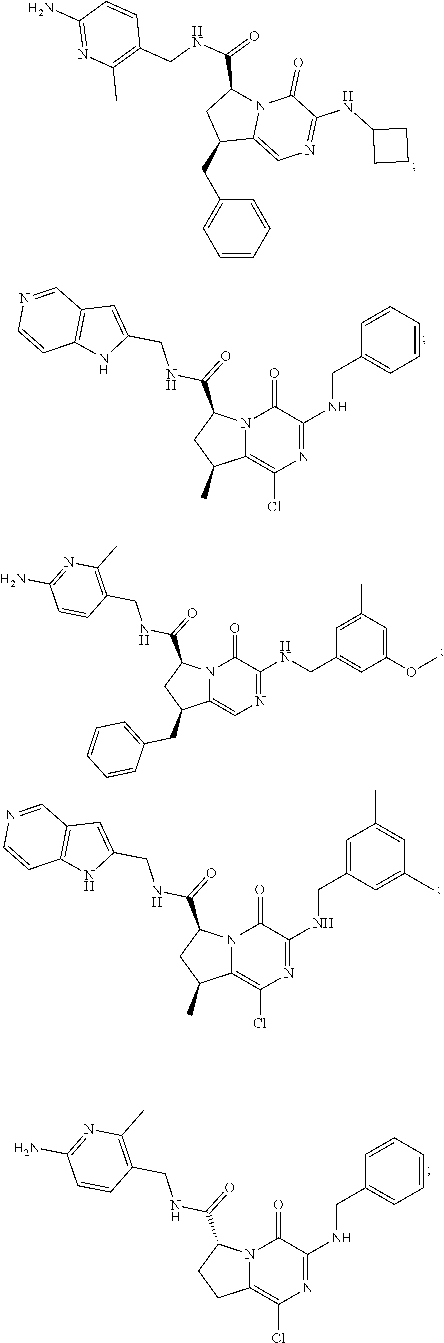 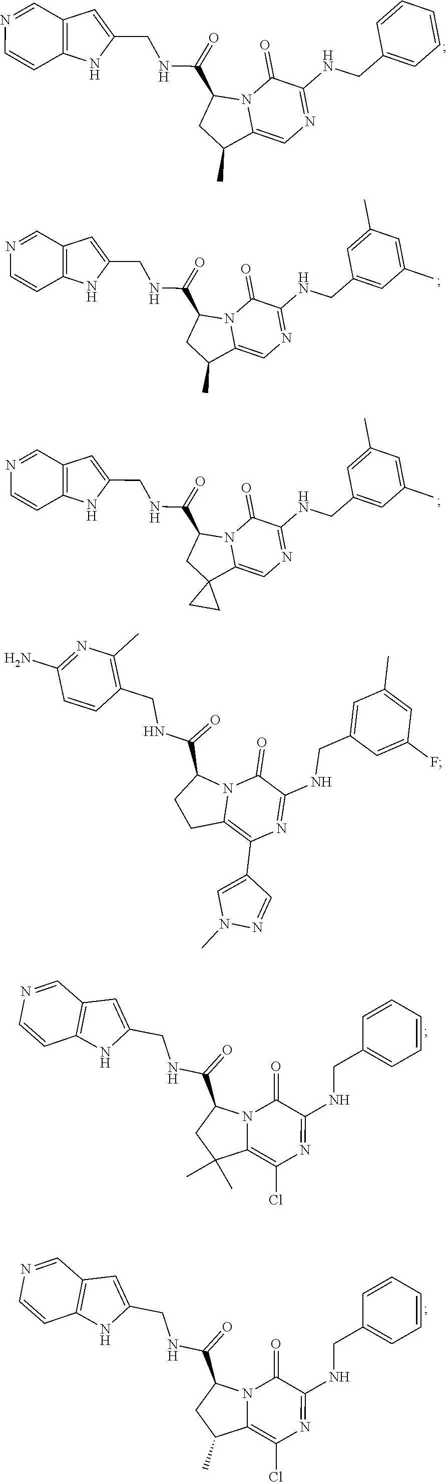 [SMILES: C[C@H]1C[C@@H](C(=O)NCc2cc3cnccc3[nH]2)n2c1cnc(NCc1ccccc1)c2=O ; Cc1cc(C)cc(CNc2ncc3n(c2=O)[C@H](C(=O)NCc2cc4cnccc4[nH]2)C[C@@H]3C)c1 ; Cc1cc(C)cc(CNc2ncc3n(c2=O)[C@H](C(=O)NCc2cc4cnccc4[nH]2)CC32CC2)c1 ; Cc1cc(F)cc(CNc2nc(-c3cnn(C)c3)c3n(c2=O)[C@H](C(=O)NCc2ccc(N)nc2C)CC3)c1 ; CC1(C)C[C@@H](C(=O)NCc2cc3cnccc3[nH]2)n2c1c(Cl)nc(NCc1ccccc1)c2=O ; C[C@@H]1C[C@@H](C(=O)NCc2cc3cnccc3[nH]2)n2c1c(Cl)nc(NCc1ccccc1)c2=O] 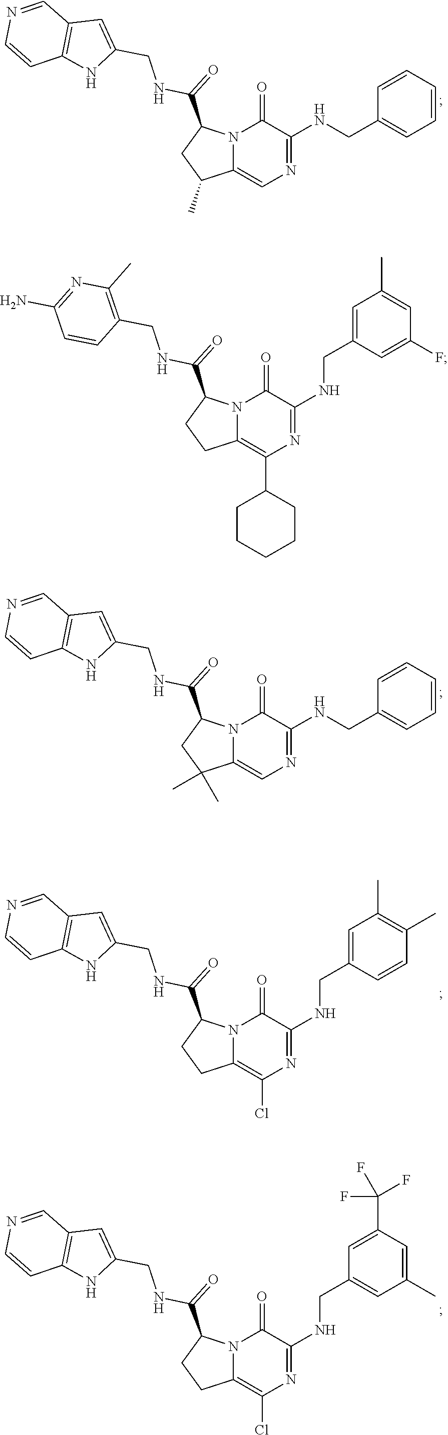 [SMILES: C[C@@H]1C[C@@H](C(=O)NCc2cc3cnccc3[nH]2)n2c1cnc(NCc1ccccc1)c2=O ; Cc1cc(F)cc(CNc2nc(C3CCCCC3)c3n(c2=O)[C@H](C(=O)NCc2ccc(N)nc2C)CC3)c1 ; CC1(C)C[C@@H](C(=O)NCc2cc3cnccc3[nH]2)n2c1cnc(NCc1ccccc1)c2=O ; Cc1ccc(CNc2nc(Cl)c3n(c2=O)[C@H](C(=O)NCc2cc4cnccc4[nH]2)CC3)cc1C ; Cc1cc(CNc2nc(Cl)c3n(c2=O)[C@H](C(=O)NCc2cc4cnccc4[nH]2)CC3)cc(C(F)(F)F)c1] 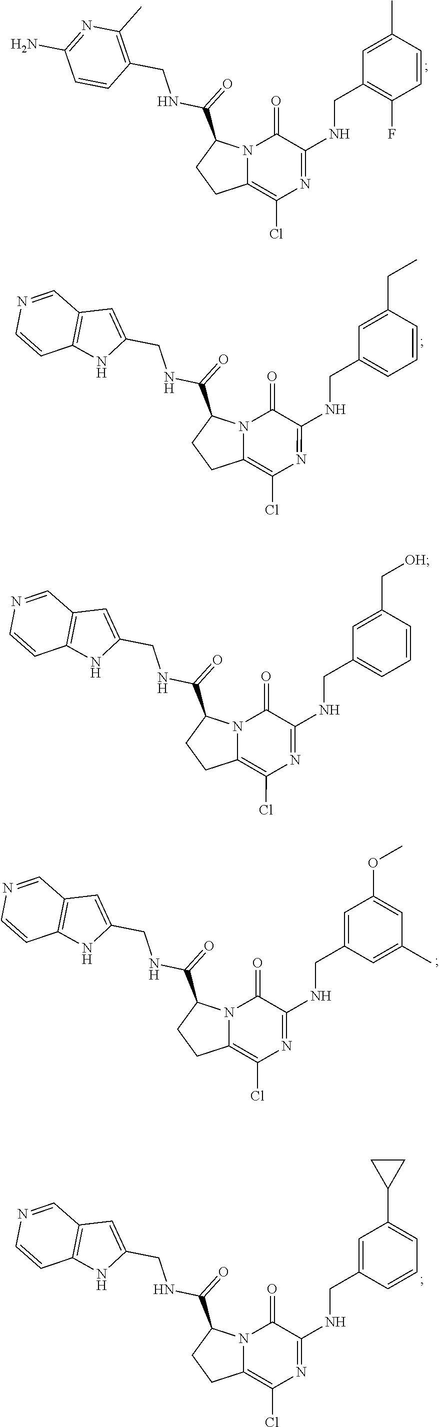 [SMILES: Cc1ccc(F)c(CNc2nc(Cl)c3n(c2=O)[C@H](C(=O)NCc2ccc(N)nc2C)CC3)c1 ; CCc1cccc(CNc2nc(Cl)c3n(c2=O)[C@H](C(=O)NCc2cc4cnccc4[nH]2)CC3)c1 ; O=C(NCc1cc2cnccc2[nH]1)[C@@H]1CCc2c(Cl)nc(NCc3cccc(CO)c3)c(=O)n21 ; COc1cc(C)cc(CNc2nc(Cl)c3n(c2=O)[C@H](C(=O)NCc2cc4cnccc4[nH]2)CC3)c1 ; O=C(NCc1cc2cnccc2[nH]1)[C@@H]1CCc2c(Cl)nc(NCc3cccc(C4CC4)c3)c(=O)n21] 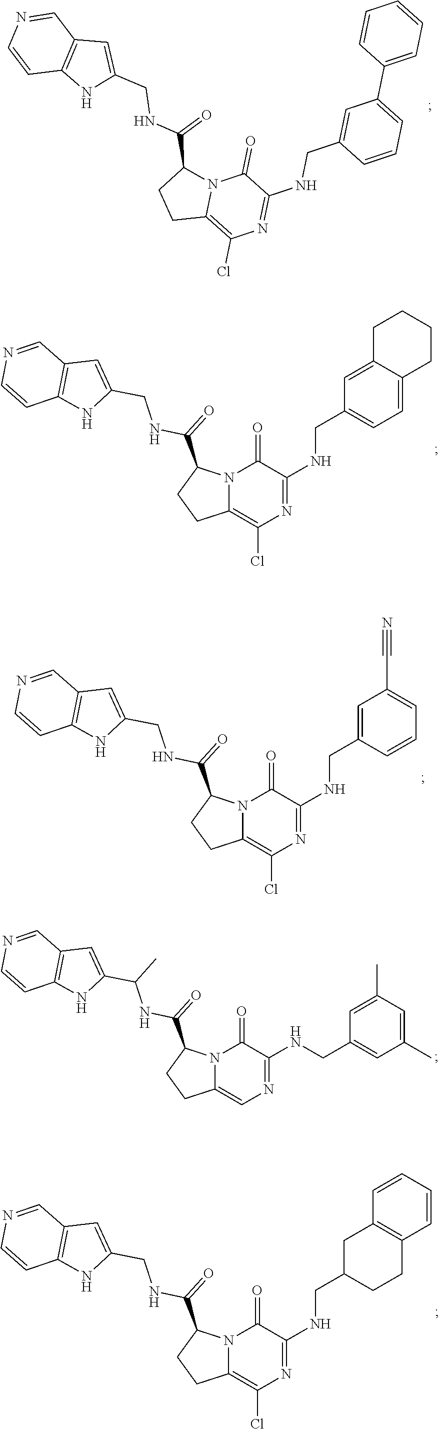 [SMILES: O=C(NCc1cc2cnccc2[nH]1)[C@@H]1CCc2c(Cl)nc(NCc3cccc(-c4ccccc4)c3)c(=O)n21 ; O=C(NCc1cc2cnccc2[nH]1)[C@@H]1CCc2c(Cl)nc(NCc3ccc4c(c3)CCCC4)c(=O)n21 ; N#Cc1cccc(CNc2nc(Cl)c3n(c2=O)[C@H](C(=O)NCc2cc4cnccc4[nH]2)CC3)c1 ; Cc1cc(C)cc(CNc2ncc3n(c2=O)[C@H](C(=O)NC(C)c2cc4cnccc4[nH]2)CC3)c1 ; O=C(NCc1cc2cnccc2[nH]1)[C@@H]1CCc2c(Cl)nc(NCC3CCc4ccccc4C3)c(=O)n21] 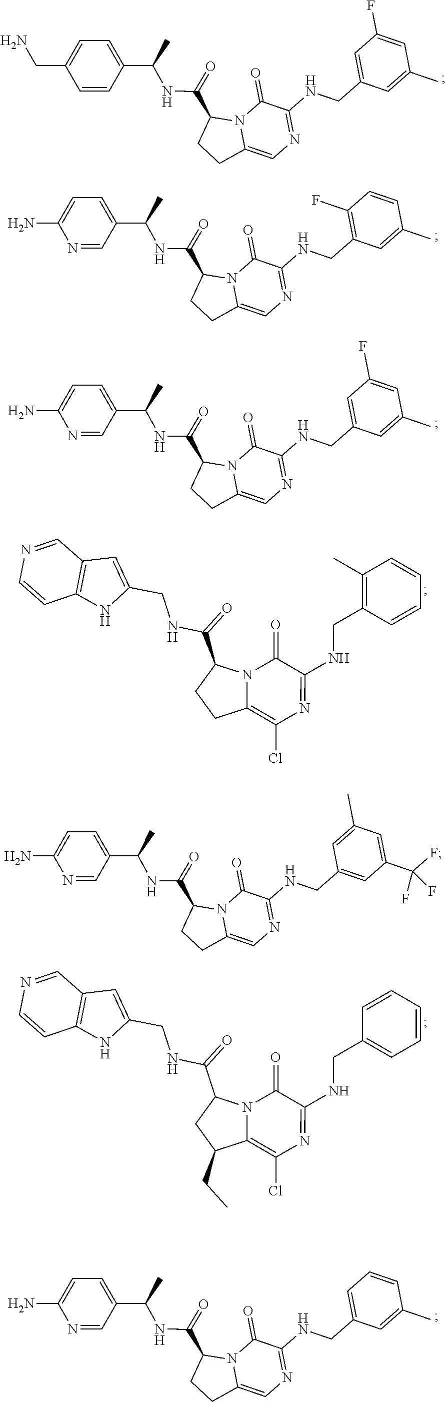 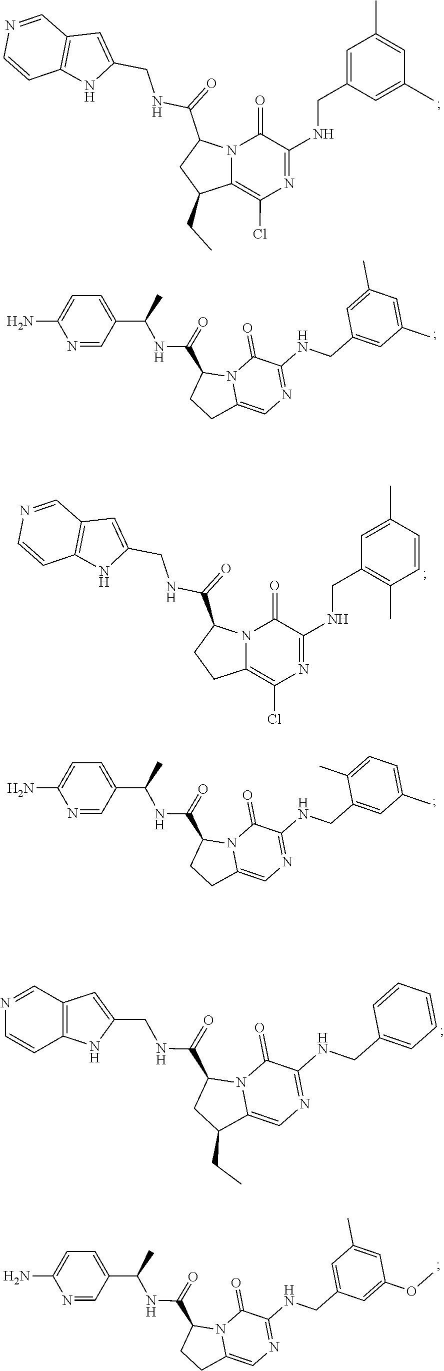 [SMILES: CC[C@H]1CC(C(=O)NCc2cc3cnccc3[nH]2)n2c1c(Cl)nc(NCc1cc(C)cc(C)c1)c2=O ; Cc1cc(C)cc(CNc2ncc3n(c2=O)[C@H](C(=O)N[C@H](C)c2ccc(N)nc2)CC3)c1 ; Cc1ccc(C)c(CNc2nc(Cl)c3n(c2=O)[C@H](C(=O)NCc2cc4cnccc4[nH]2)CC3)c1 ; Cc1ccc(C)c(CNc2ncc3n(c2=O)[C@H](C(=O)N[C@H](C)c2ccc(N)nc2)CC3)c1 ; CC[C@H]1C[C@@H](C(=O)NCc2cc3cnccc3[nH]2)n2c1cnc(NCc1ccccc1)c2=O ; COc1cc(C)cc(CNc2ncc3n(c2=O)[C@H](C(=O)N[C@H](C)c2ccc(N)nc2)CC3)c1] 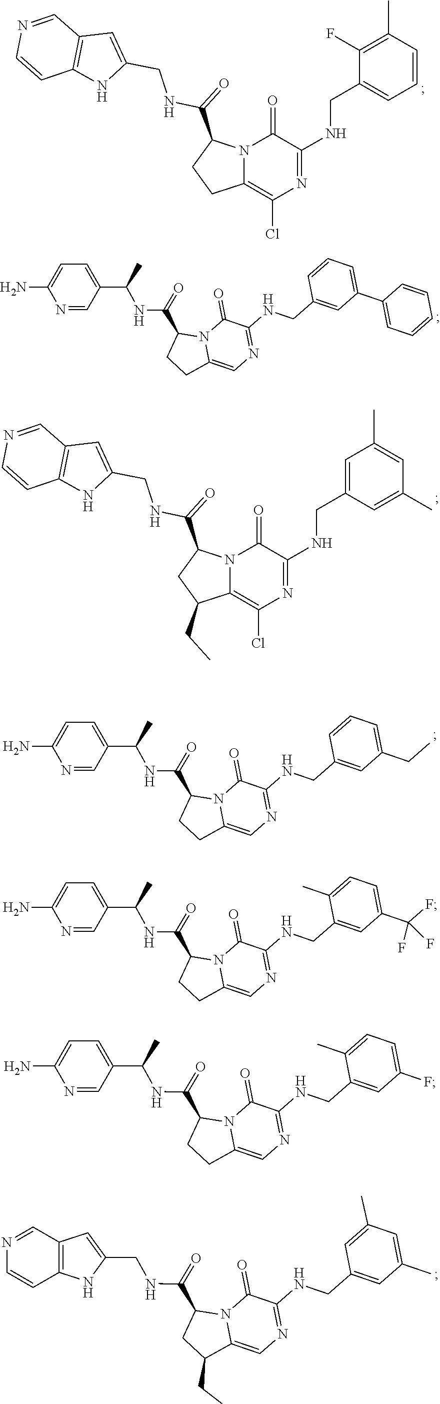 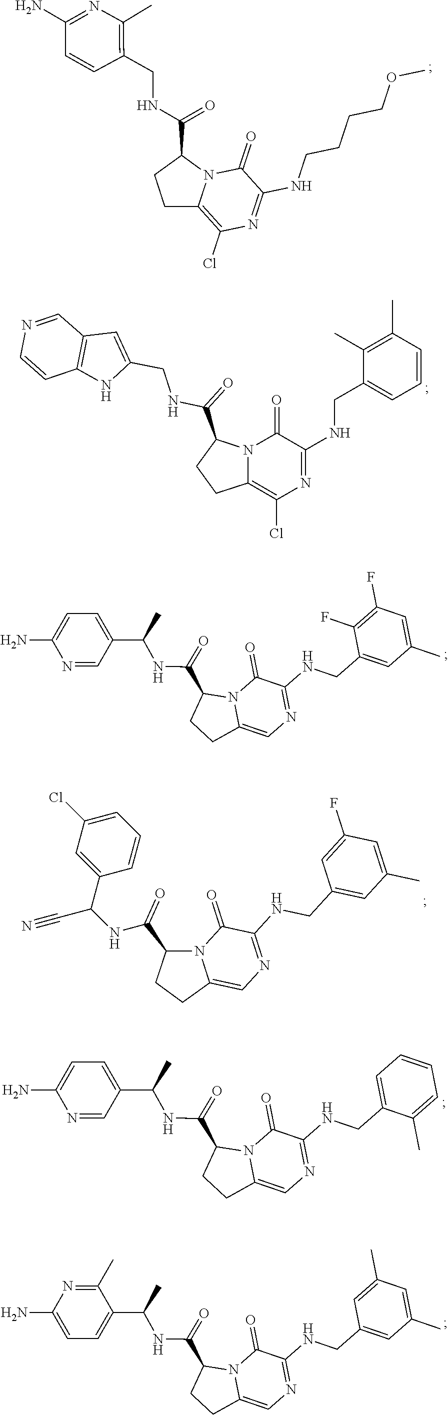 [SMILES: COCCCCNc1nc(Cl)c2n(c1=O)[C@H](C(=O)NCc1ccc(N)nc1C)CC2 ; Cc1cccc(CNc2nc(Cl)c3n(c2=O)[C@H](C(=O)NCc2cc4cnccc4[nH]2)CC3)c1C ; Cc1cc(F)c(F)c(CNc2ncc3n(c2=O)[C@H](C(=O)N[C@H](C)c2ccc(N)nc2)CC3)c1 ; Cc1cc(F)cc(CNc2ncc3n(c2=O)[C@H](C(=O)NC(C#N)c2cccc(Cl)c2)CC3)c1 ; Cc1ccccc1CNc1ncc2n(c1=O)[C@H](C(=O)N[C@H](C)c1ccc(N)nc1)CC2 ; Cc1cc(C)cc(CNc2ncc3n(c2=O)[C@H](C(=O)N[C@H](C)c2ccc(N)nc2C)CC3)c1] 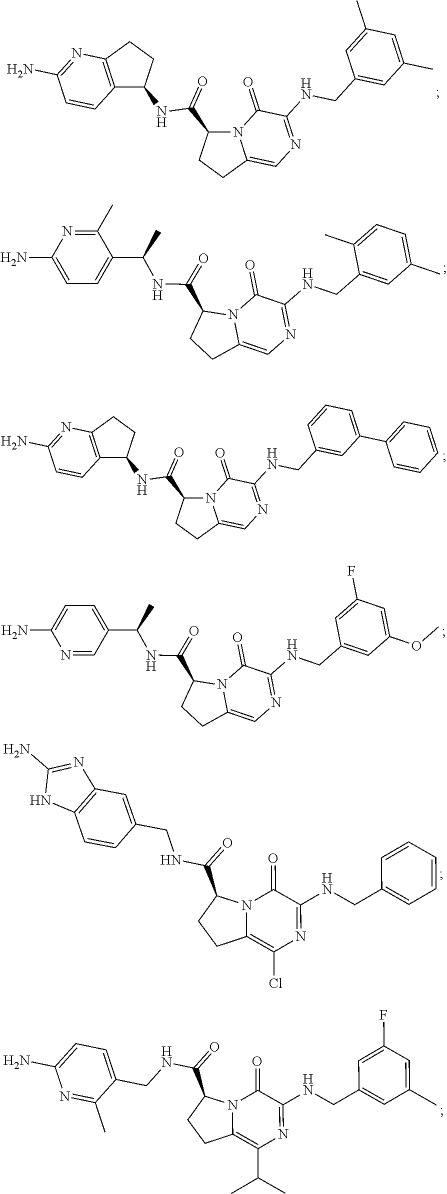 [SMILES: Cc1cc(C)cc(CNc2ncc3n(c2=O)[C@H](C(=O)N[C@@H]2CCc4nc(N)ccc42)CC3)c1 ; Cc1ccc(C)c(CNc2ncc3n(c2=O)[C@H](C(=O)N[C@H](C)c2ccc(N)nc2C)CC3)c1 ; Nc1ccc2c(n1)CC[C@H]2NC(=O)[C@@H]1CCc2cnc(NCc3cccc(-c4ccccc4)c3)c(=O)n21 ; COc1cc(F)cc(CNc2ncc3n(c2=O)[C@H](C(=O)N[C@H](C)c2ccc(N)nc2)CC3)c1 ; Nc1nc2cc(CNC(=O)[C@@H]3CCc4c(Cl)nc(NCc5ccccc5)c(=O)n43)ccc2[nH]1 ; Cc1cc(F)cc(CNc2nc(C(C)C)c3n(c2=O)[C@H](C(=O)NCc2ccc(N)nc2C)CC3)c1] 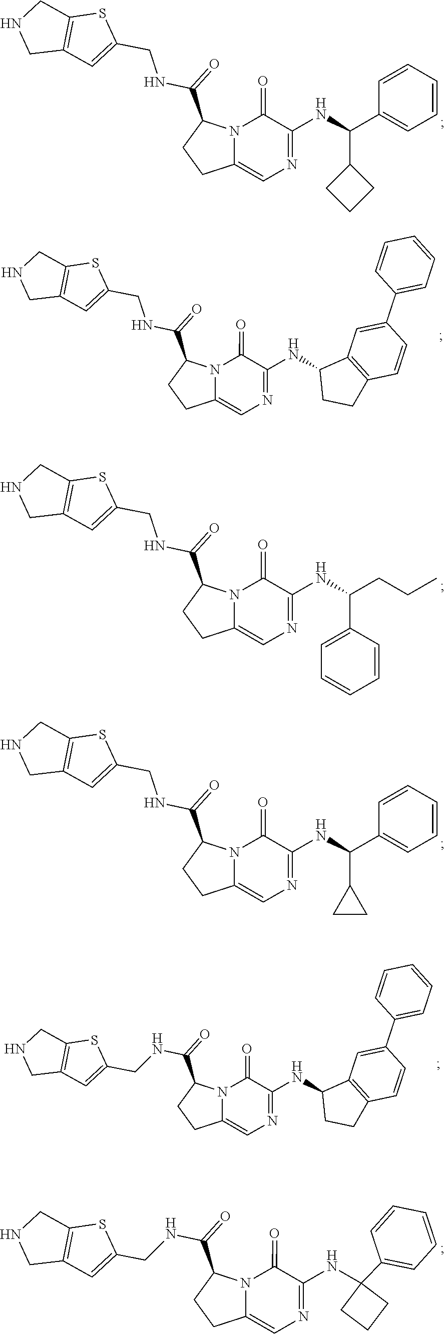 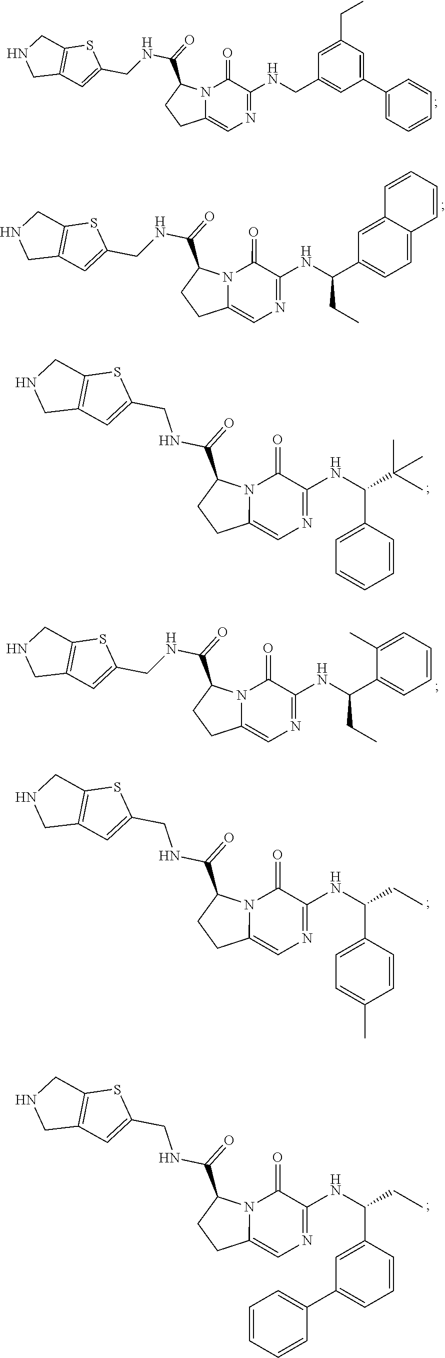 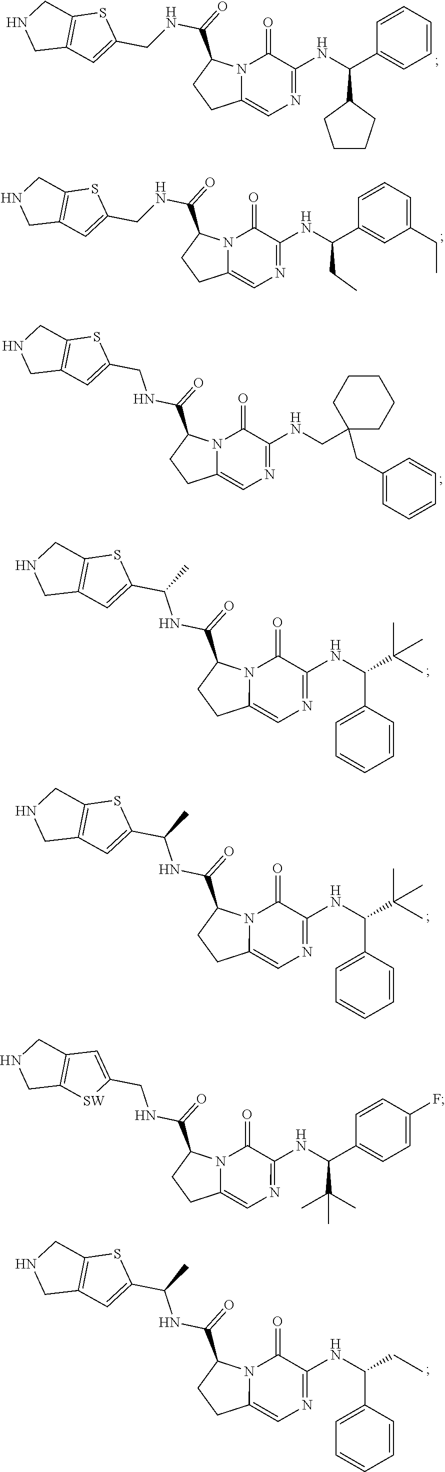 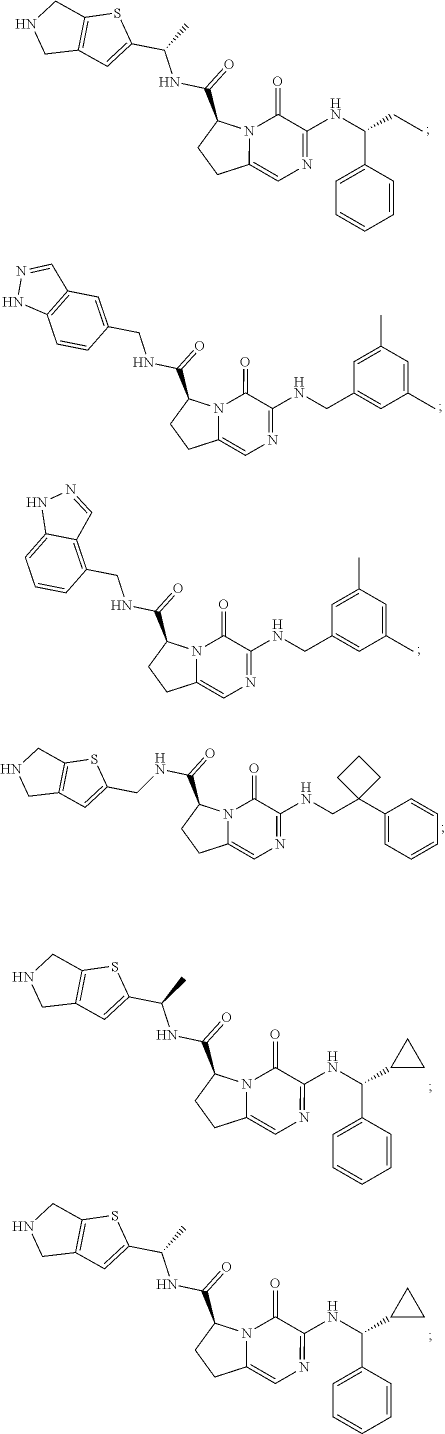 [SMILES: CC[C@@H](Nc1ncc2n(c1=O)[C@H](C(=O)N[C@@H](C)c1cc3c(s1)CNC3)CC2)c1ccccc1 ; Cc1cc(C)cc(CNc2ncc3n(c2=O)[C@H](C(=O)NCc2ccc4[nH]ncc4c2)CC3)c1 ; Cc1cc(C)cc(CNc2ncc3n(c2=O)[C@H](C(=O)NCc2cccc4[nH]ncc24)CC3)c1 ; O=C(NCc1cc2c(s1)CNC2)[C@@H]1CCc2cnc(NCC3(c4ccccc4)CCC3)c(=O)n21 ; C[C@@H](NC(=O)[C@@H]1CCc2cnc(N[C@@H](c3ccccc3)C3CC3)c(=O)n21)c1cc2c(s1)CNC2 ; C[C@H](NC(=O)[C@@H]1CCc2cnc(N[C@@H](c3ccccc3)C3CC3)c(=O)n21)c1cc2c(s1)CNC2] 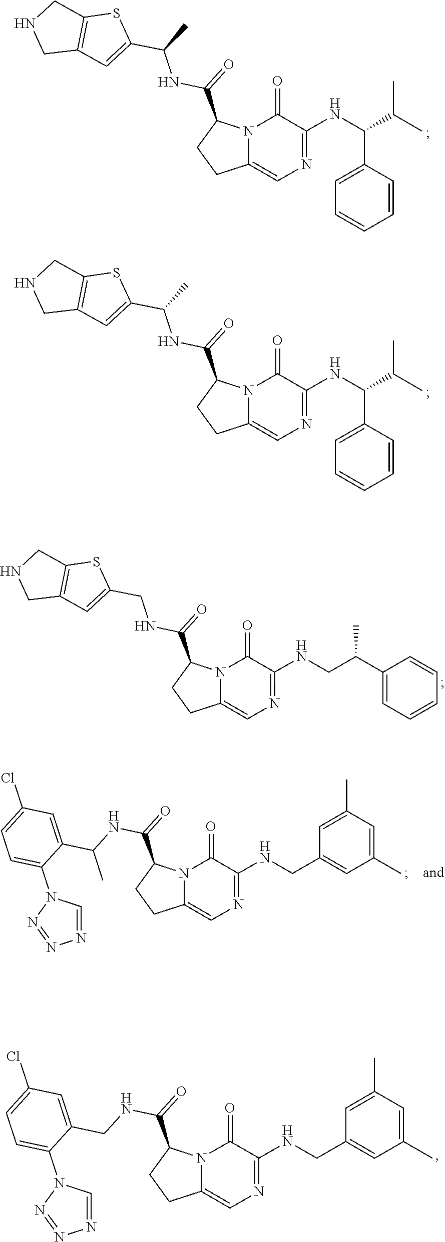 [SMILES: CC(C)[C@@H](Nc1ncc2n(c1=O)[C@H](C(=O)N[C@H](C)c1cc3c(s1)CNC3)CC2)c1ccccc1 ; CC(C)[C@@H](Nc1ncc2n(c1=O)[C@H](C(=O)N[C@@H](C)c1cc3c(s1)CNC3)CC2)c1ccccc1 ; C[C@@H](CNc1ncc2n(c1=O)[C@H](C(=O)NCc1cc3c(s1)CNC3)CC2)c1ccccc1 ; Cc1cc(C)cc(CNc2ncc3n(c2=O)[C@H](C(=O)NC(C)c2cc(Cl)ccc2-n2cnnn2)CC3)c1 ; Cc1cc(C)cc(CNc2ncc3n(c2=O)[C@H](C(=O)NCc2cc(Cl)ccc2-n2cnnn2)CC3)c1]
or a stereoisomer, tautomer, or pharmaceutically acceptable salt thereof.

110. A pharmaceutical composition, comprising a compound of claim 1, or a stereoisomer, tautomer, or pharmaceutically acceptable salt thereof, and a pharmaceutically acceptable carrier or excipient.

111. A method for inhibiting MASP-2 in a subject, comprising administering to the subject a compound of claim 1, or a stereoisomer, tautomer, or pharmaceutically acceptable salt thereof, in an amount effective to inhibit MASP-2.

112. (canceled)

113. (canceled)

114. A method for treating a disease or disorder treatable by inhibiting MASP-2, comprising administering to a subject in need thereof a therapeutically effective amount of a compound of claim 1, or a stereoisomer, tautomer, or pharmaceutically acceptable salt thereof.

115.-153. (canceled)

154. A method for inhibiting MASP-2 in a subject, comprising administering to the subject a compound of Formula (I): 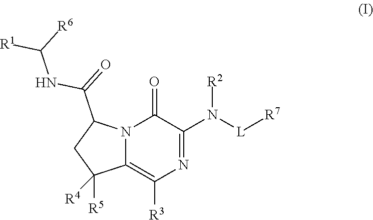
or a stereoisomer, tautomer, or pharmaceutically acceptable salt thereof, wherein:
R¹ is selected from a substituted or unsubstituted C₆-C₁₀ aryl and a substituted or unsubstituted 5-10-membered heteroaryl consisting of a 5-10-membered monocyclic or bicyclic ring system comprising at least one aromatic ring and one to six heteroatoms selected from the group consisting of nitrogen, oxygen, and sulfur, or
R¹ and R⁶, together with the carbon to which they are attached, form a C₃-C₆ cycloalkyl fused to a 5-10-membered heteroaryl or C₆-C₁₀ aryl, wherein the 5-10-membered heteroaryl or C₆-C₁₀ aryl, of the C₃-C₆ cycloalkyl fused to the 5-10-membered heteroaryl or C₆-C₁₀ aryl, is substituted or unsubstituted, and wherein the C₃-C₆ cycloalkyl, of the C₃-C₆ cycloalkyl fused to a 5-10-membered heteroaryl or C₆-C₁₀ aryl, is substituted or unsubstituted;
R² is hydrogen, or R² and L, together with the nitrogen to which they are attached, form a substituted or unsubstituted 3-6-membered heterocyclyl;
L is —(CR^{8a}R^{8b})ₙ—, wherein each —(CR^{8a}R^{8b})— is the same or is different;
R³ is selected from the group consisting of hydrogen, substituted or unsubstituted C₁-C₃ alkyl, substituted or unsubstituted C₃-C₆ cycloalkyl, substituted or unsubstituted 5-6-membered saturated or unsaturated heterocyclyl, substituted or unsubstituted phenyl, substituted or unsubstituted 5-6-membered heteroaryl, halogen, and cyano;
R⁴ and R⁵ are each independently selected from the group consisting of hydrogen, substituted or unsubstituted C₁-C₆ alkyl, and substituted or unsubstituted benzyl, or R⁴ and R⁵, together with the carbon to which they are attached, form a substituted or unsubstituted C₃-C₆ cycloalkyl or a substituted or unsubstituted 5-6-membered cycloalkenyl;
R⁶ is selected from the group consisting of hydrogen, C₁-C₃ alkyl, C₃-cycloalkyl, and cyano;
R⁷ is selected from the group consisting of hydrogen, substituted or unsubstituted C₁-C₃ alkyl, substituted or unsubstituted 5-6-membered heteroalkyl, substituted or unsubstituted C₃-C₆ cycloalkyl, substituted or unsubstituted C₃-C₆ cycloalkyl fused with aryl, substituted or unsubstituted 3-6-membered heterocyclyl, substituted or unsubstituted C₆-C₁₀ aryl, substituted or unsubstituted C₆-C₁₀ aryl fused with C₅-C₆ cycloalkyl, substituted or unsubstituted 5-9-membered heteroaryl, substituted or unsubstituted 5-9-membered heteroaryl fused with C₅-C₆ cycloalkyl, alkoxy, and halogen, and wherein when R⁷ is hydrogen, n is 0;
R^{8a} and R^{8b} are each independently selected from the group consisting of hydrogen, C₁-C₄ linear alkyl, C₃-C₄ branched alkyl, substituted or unsubstituted C₃-C₆ cycloalkyl, hydroxyalkyl, and alkoxy; or R^{8a} and R^{8b} together, with the carbon to which they are attached, form a substituted or unsubstituted C₃-C₆ cycloalkyl or a substituted or unsubstituted 3-6-membered heterocyclyl; or
n is 3, one of R^{8a} or R^{8b} from a first carbon is a bridging —CH₂— to a third carbon, and one of R^{8a} or R^{8b} from the third carbon is absent, to form a 1,3-cyclobutylene; and
n is 0, 1, 2, 3, or 4, and
wherein when one or more chiral center is present, each chiral center has (R) stereochemistry, (S) stereochemistry, or is a mixture thereof.

155. (canceled)
